SEQUENCE LISTING (486 sequences)

SEQ ID NO: 1 - DNA, 25 bp, artificial sequence
  Primer
aattaattaaccctcactaaagggt

SEQ ID NO: 2 - DNA, 25 bp, artificial sequence
  primer
agctgtaatacgactcactataggg

SEQ ID NO: 3 - DNA, 361 bp, Homo sapiens
ttagcacaaacacatttatttattaaccaaagggatgatcctaattaatccaacacactttgaaatagctgcatgtaaaatgtttatgataaagataattgaacacagtagtgccgaaacagcagaaagagacagtatggagaattgctcattggactgagcttggtcattctcttaattaactcctgtccaaagtgatgatggaatttttattctactttttcatagatccgagtacaggtgacattgttcatgacacactccaccactaatttcccatctttcaattttcttgttattgtgctttccttcccatcccactcctgatgctgaaccaatgcaccatctgtaaagttgcaga

SEQ ID NO: 4 - DNA, 358 bp, Homo sapiens
  misc_feature (62)..(62): a,t,g,c
gaggtattgatgactttattattctgcaggtacatgtccaggggcccagcctctgggcaagntaactcagctactctttgtggctttcttcatggctttttttgtgggctgccacgcccatctttatcaccagaatgaggaactcctggaagttaactgcaccatcagtgttgatatccaactctttgaaccagacgtctgcaccctttttcctgatatactgaggacactcggtctctagcaatttcttcaggtcatccctgtagacggcatggaaattcccctttatcaagggagtacttgtggtaaacgtcgatgatagagttcaaggctttctacagctcggtcaacatgatng

SEQ ID NO: 5 - DNA, 409 bp, Homo sapiens
tttttaccaaaaacgcaggggatttatttgaggtttgggtgaaaaataatcttgtgggtggtggtaggccgacagatggggacaggaagctgtggacgaaagccccaggtcccgtgggagaggtgacagcagcaggggcacgcagccacgtgggtccccaagggaatgtgaaggcggagggctccaggcgaactggggattaaacaaatatttacaggcagcagggaagtgcccagcgcacgtgacgggggcggggcgggactttggggagggcggggcctaacggtatcgagcgagccggttgtagacgtggtccaggtttctgcacaggaatatcgagagcgtcatgaacccgagctcgcggtaaatgtggctctctgcagggacagcgcatatggtctgcactgtg

SEQ ID NO: 6 - DNA, 257 bp, Homo sapiens
  misc_feature (82)..(82): a,t,g,c
taagtagcaggtttcattttaaaacaaaaaaggttagtgaagactctgtctttcaaaacataaaaatctgcgataaaaccanttattccatacagtgactacggtcagttctgagaaatgacacccaggttggcgatgtgtctcatggttggccttccatggggacagttccaggggtggtncatctcccccatgtgggtgatcagtttcttcatctcgcttgtgttgagagcagtcccaatcatcaccgncttncg

SEQ ID NO: 7 - DNA, 344 bp, Homo sapiens
  misc_feature (336)..(336): a,t,g,c
gaaatggaaatcattggtcagtttaacctggggatttataataaccaaactgaatgaggatatcttcatagtggaccagcatgccacggacgagaagtataacttcgagatgctgcagcagcacaccgtcttccaggggcagaggctcatagcacctcagactctcaacttaactgctgttaatgaagctgttctgatagaaaatctggaaatatttagaaagaatggctttgattttgttatcgatgaaaatgtcatggatttttctcaaaattgtattcttttagctccagtcactgaaagggctaaactgatttcccttgccaactggtaaancctgggnc

SEQ ID NO: 8 - DNA, 361 bp, Homo sapiens
  misc_feature (320)..(320): a,t,g,c
agttaaatagtacagaagacagtttactgtacaagcaagttgtgcgttaaaaacaaacaccaagcaaacgataggcaaagagttttccacccagctccatcctctcgccagctctgggatggttttacatcagatgagtgcagcaggtgtcacacctcagcatgacaatatgtcacaaaagattggtacccactactgacaggctcacagtaacactatatcaaaacgtcttcctttcctcgtgcttcctacatcagtgtgtttgcctaggtacaactttaacggcggccttgttaaattaggggccctactttttaccnggccgggggttntcttggttnccactttnggggnctttaca

SEQ ID NO: 9 - DNA, 418 bp, Homo sapiens
  misc_feature (1)..(1): a,t,g,c
ntttcggcacaggaaatctgccctacaaaatgctcttcattggttgtctctgtgagagcactgtccccacccaacctgtcacaacggccagaaccatacaccagagacacactggcaggttaggcagtccttctggtgatcctattccattccctcctgctgcggtttctcttggcctgtcctcactggaaaaacagtctccatctcctcaaaatagttgctgactccctggcacccaaggggccttttccatggccttcttagggaaggcaggtatggaatccatttgttccttgttagtttntttccctcctgtttntctgggttattaggntgggtcccagggttcaggcgtgggggaggggcacctttnggggtttcccagttggcccaggatttttgttaggtcttcattnta

SEQ ID NO: 10 - DNA, 413 bp, Homo sapiens
  misc_feature (343)..(343): a,t,g,c
ttgctctttattttctacataataaaacttaatcactggggcacagggatgcacgcttgtaatcccagctactggggaagcttaggcaggacgactgtttgagctcaggagtttcagactgtagtgcattatgactgcacctgtgaatagccactgcattccagcccttagcgagaccttgcctcaagactttggggaggctatttcttaagcatatataaccaatccatttcagtgtcactatgggtgcttgtcaaggcccaatgctatccaaaaacacaggtaattattccctccctattttgggcaggattctgctggattctgattctgggccaggggnggaagggaggggngccattttaagggggagactttattacntgctttgctccatcactgtgctcctaaat

SEQ ID NO: 11 - DNA, 344 bp, Homo sapiens
agtaaaccagaagatttgaaggtggttaagaactgtgcaaataccacaagatcattttgtgacctcacagatgagtggagaagcacacacgaggcctatgtcaccgtcctagaaggattcagcgggaacacaacgttgttcagttgctcacacaatttctggctggccatagacatgtcttttgaaccaccagagtttgagattgttggttttaccaaccacatttaatgtgatgggtgaatttccatctattgtttgagggaaggaattacagttttgattttttctctcgtcattggaaggaacagttcagaggggatttgtttaaggaaggcattaaaccc

SEQ ID NO: 12 - DNA, 521 bp, Homo sapiens
tttttttttttattaagaaagcagtttatttcttaataactgaatgtatattattttacatataattaatgatcagagagaaataacagttgctgagaacgtaaatttatgttgtactttagaattgaacagatacaaccacagattcataccaaatgcattacttttagattattaacatattcttttacataatttcatttcacatatatggagtccaaccaagatacatctggcatagtaagttttcatcagtagcttcttgtataaggtaatgcacatgtccttcaatagataacggcagtcctgtcactctatttcgagtcttgattacaccttgtagtcgctgctcaatgtcaagaacatgggtcttggccttttcattgacaacttctccagtttcattcagtggcgctttggaatgccctttcactgggttactccattccacaagaggatacatgtagaaaagttcttaagaccctcattaaaggctctcgctgatcacgcatcagcctcat

SEQ ID NO: 13 - DNA, 450 bp, Homo sapiens
ccttgtgttgcaaagcctttaattagaatgtttgtattttttacatcatgcataacttcacatttgtgattaattagtaattatttcaatacttgtaagctcatctgcctcagatttaatcataatacatgaattaaattaatcaaattaaggaacagcaatttagaaagaaacacactttaagaaatcaaaattctcaattcaggcagtctgtttctatcatttggtattctactcctttaaaaatttcatattgcccaacaaaaagtggttatttttactgtttttggagatgactgaacagatgaaggcatcagatgccttcatcagctggtattttgcctaagatctatttaagataaccttttcttatattttttacttaatattgggcataatcgtatcattcaagattaagtctgggttatttctttgataagaactgaatgt

SEQ ID NO: 14 - DNA, 459 bp, Homo sapiens
tttttttttttttttttttattttttttttttttttttttttttttttttgacaaattcactttaaaatgttttccacatcaactacttaaaagaagttacagcaatatgatcttccactgctcaggaagacagagtgaacttggatatgatgaatattaaaaacaaacatacagagcacctgggtctggacttgtgggctaaagaaatgcttcctgaatttgtccgaggtgatgtgttgagcaaccgtgccacagttcaccagcagtgacactctagttcttcagtatattcctgttgtgaacctatttctttgaatttaatcatctgggatgattgctgcagtgtgagtctttagcattttgaaaatcccacagaagagattaagaaggttcctcttcccagaagacactcatataaagcatgcttctatagggttaactgttcagaaattgtaagg

SEQ ID NO: 15 - DNA, 273 bp, Homo sapiens
ggaaaaaaattaagtgatttttatttccaacagttgtaaattaaaattgtacaacatgttttctaatctatgtctcatgtgtcttgctgtatgactgaagacaatgaatgtatttttaaaaattatggcagacacaatatacttggtatttacaatggcctgtgacaaatataatacctggaaataatttctaattttccacttattattgctatttagtcacagaaaagattaacactctccaagaaattttagcatatgtcatatatagat

SEQ ID NO: 16 - DNA, 515 bp, Homo sapiens
tttttttttttgacccatgaccatgaagtttatagtttgactatctgtaggaagggtacaaaaatacatcatgcaacaaggtcattcatttggaaatgtaatgctttctgaaaaacttgagcattcacttttcttcacttgcctgttctaataagagaggtttggtgtggtgatttttcaatagaaaggatgttaccaatatataaataacataaaaggatcacatgagaattaaagtgaagtaacttaacatacaattttcattctaatcttatacaataatttttcttgactattgatagatcacaataaaggtatgctgatgatataagttattttaagactttggggaaaaaggcttccgtctctggcattgaccttattgtggcggaccctccaaaatcttcagctctgcaccagcaggtatgctgagtcctgtatgatgacataaaccacaggagtccaggaagcaaaaccaccacagcgactggctcagatctcagatgtgccagatg

SEQ ID NO: 17 - DNA, 453 bp, Homo sapiens
  misc_feature (196)..(196): a,t,g,c
aatcagcaatgttttttttatttaaggcagaattagggaaaggctatgccctccactccccctcctcctaccactcagtcctatcccaagtcaggcactggaacctgctgaaaaggctgatgaggggctgacaggagtggggcagggttcatggacctttacctcagagcagcttaccctgagctatacccacatntcaaggatttgggggcgtctagatctcatctaagggagacacaacttttctggtcctcagatggaaagctgggggaaggtaccagtatttacccttccataaaaactttggaggtctttaggaggntacccctcaggagggctaggagaggantgacaaatacagagggatagaaggtcccntggcctggttntgccctcacacagggtattgggttnttccagggnttgccactcccgttgccacaccaccntttg

SEQ ID NO: 18 - DNA, 453 bp, Homo sapiens
  misc_feature (193)..(193): a,t,g,c
gcggcacgaggtcgaacctgagccttttgagaattgcctgctacggcctggcagccctgcccgagtggtccagtgcatttcagagctgggcatctttggggtctatgtcaggcaggaaaagacactcgtgatgaacaagcacgtggggcatctacttcgaaccaaagccatcgagcatgcagatggtggtgtngcagcgggagtnggcagtcctgggacaacccataccctgtgtgagggcacaaccaggccacgggaccttctatcctctgtatttgtcattcctctccttagccctcctgaggggtatcctcctaaagacctccaaagtttttatgggaagggtaaatactggtaactttcccccagcttttccntcttgagggaccagaaaagttgntgtttncctttagatgagnttttggaaggcccccaaatcctttnagatttggg

SEQ ID NO: 19 - DNA, 415 bp, Homo sapiens
catttcaccactttgaaatcttggaaccactgatgtgatatcccacagagagggcaagacttcactgtgaagtcaccaaaggaactcctgcccttctaggtgccacttgagtaataactcccaactcactggtcctctaaacctgcagcagggagatggtgtgctaggtagctacatgggcaggagaagaaatggaaacaaattcccggagaatgtttttggccatctcgatgttgttctgggcaatgaccaaagctttctggatgtcctggtaggagtacccctgactcatgaggttctcgatctcactggagaactgaggtgaggcggtggcagcagaggcggcaggaccactaccttgctgaaagctgccagctttccgttcagagttgattctccgcgggaatggttttgg

SEQ ID NO: 20 - DNA, 437 bp, Homo sapiens
  misc_feature (82)..(82): a,t,g,c
ggaacagggtggacactgtgcaggcttcagcttccactccgggcaggattcaggctatctgggaccgcaggacttgccaggngcacagccctggctcccgaggcaggcaggcaaggtgacgggactggaagcccttttcanagccttggaggagctggnccgtccacaagcaatgagtgccactctgcagtttgcaggggatggataaacagggaaacactgtgcattcctcacagccaacagtntaggtcttggtnaagccccggcgctgagctaagctcaggcttttccaggggagccacgaaactncaggtagtgatgtgcaagagtccatcctgcagttttccagcaatnagaaactcctcgttngcggtttttggggaccnttggaagttntccgcagacatttttccatgggccgggttttaagacgaacc

SEQ ID NO: 21 - DNA, 466 bp, Homo sapiens
  misc_feature (4)..(4): a,t,g,c
gccncagatccagcgcccagagagacaccagagaacccaccatggccccctttgnagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttncacaggtcccacaaccgnagcgaggagtttctcattngctggaaaactgtaggatggacttcttgcacattnactaccttgcagttttngtgggttccctgggaacagtctgaggtttagttnagcggtnggggtt

SEQ ID NO: 22 - DNA, 453 bp, Homo sapiens
tttttttttgaacttttcgtgagtgtgtttattaactcttactccccacgggcaaggctgggttttggtgagggaaagaaaagaccctcgttgaggacattgctgggtaaaaagccttcagagagctacctactctttgtgggtgtggtaatgggcagccttcagcgcaatggctaccagggatatgaattcttgaaagtcgacctgttcatcttgattagcatccaggccttggaatatttcatcaatgacagctttatctttgatattcttgatggtgtttgcaagctcctttgtaagcagctgcttcagctcacccttagagagggtgtcaaaatgccccttccgaactgagtattggtggaagatattgacaattccctccagatgctcttcaagttttgtcatcttcccagcctaatgttaacccctcaatgcacaggaatgtggcct

SEQ ID NO: 23 - DNA, 544 bp, Homo sapiens
agttgtaaacaccaggcggctttattggtagcattgcaatctgggggtggagcagctttactctgaccacactcaccctctcgctcacactcacactcacacacaccccaggccctgtcctggctggtcaggtctggtgcagaggagctggaagcctggtccttccttctggtcctcggttccccaggttaggtaataattcacaatgcttgactcggactccgggctccctctgcacggtcataggttaactgctgcggcgcttcatcttggctgaggtcctctgcagtctctggatgatgcgttctacccagggctggtctgggggtgcacagagctggcggcccctcagtgtggtgaacactacagcaggcaccctgcagccatccttgatgagaaggtagtggaagttcctcacgatgtacccagggatgggtttctgggtcacagacaggcagcagtcttcagcatcattggtgccactcagagttggggctggggaagtccagagaaccagcaggctgagggccagtagcagggccat

SEQ ID NO: 24 - DNA, 472 bp, Homo sapiens
  misc_feature (18)..(18): a,t,g,c
gtgttttttgtagagatnctntnttgccatgttgccggcactggtcttcaattctgggctcagacagtccacccacctcaccctcccagagtgctgtgattacaggcatgagccaccatgtccagcctaaggtcttctttttaaaactttgactatattatgatttccagagatttacaaaaatgcattaaagatagggtatttggtaaatagagatagaaatagattttaattaccctttgtcagtcaaatacagatccctattcttaagcatattcttatacaatatctcattattttaaaggaccctaagtgtgaccagaccatatttaaaatgagatgaaatatataagaggtactgatgttgtgatttgtaaaacatcacaanttaantatgaggctttttttttagtgtncaagtttttaaggtngggaaatcctntccagtttataaggtagccaggttaaaatt

SEQ ID NO: 25 - DNA, 254 bp, Homo sapiens
  misc_feature (7)..(7): a,t,g,c
ttgtttnttttcttgaatatttttgacctgcagttggttgaatctggggatatataatctatggatacagagtgctgactacatatccatattttatctagctatcttataaactgaaaagatttccaactnaaaaacatgacactaaanaaaagctcatattaattgtgatgttttacaaatcacaacatcagtacctcttatatatntcatctcattttaaatatggtctggtcacacttagggtcctttaa

SEQ ID NO: 26 - DNA, 435 bp, Homo sapiens
  misc_feature (1)..(1): a,t,g,c
nnaataatangtggagaaatttttattactttgaatgttttagaatgcaggtagaagagacccggagctcaaatagcttaaataagtaggaaatctattggctaatacaactggaaatgcggagatagggcaggctgcagggctggtggctcagggctcagggggccccaactctctgtgctgctctgaggcactgccttaaatctcaggttggcagcacaaagctgctgagagtcccagtgtcacgcccagaccccacaatgccaggggaggaagacaggttctatctaaggagagatcttccatccccacctctgcggacttttactcacttctcaggggacctgcatggggatctcagggcccattcctgaaggcagttntttgggcagacagagtgggaataagtnttttacagcagggcagggcagcntt

SEQ ID NO: 27 - DNA, 498 bp, Homo sapiens
  misc_feature (26)..(26): a,t,g,c
gaccctggagcacttctaatgtatcnacccatggagtcattgttctaataatcaccaattcagactcagatcctcgtggtctatggagcatgctgcttgctgtctgtgcagctcccatttccccttcttcctgatagacttggagctgtgtgcctccactccaaggctgcctgcctgctgtaaacactattccactctgtctgccaacaactgcttcaggaatgggcctgagatcccatgcaggtccctgagaagtgagtaaaagtccgcagaggtggggatggaagatctctccttagatagaacctgtcttcctgcctggcattgtggggtctgggcgtgacantgggactctcagcagctttgtgctgccaaacctgagatttaaaggcagtgctcagagcagcacagagagttggggccccctggagcctgaggcaacagcctgcagcctgccngatctncggatttccagttgtatttaggcaatagattttc

SEQ ID NO: 28 - DNA, 278 bp, Homo sapiens
gcgtgaaacttggtgaatctttattaaactagggtccaccccaggaggacggctggggcggggacagggtctcccgctgcaggctgcgcggaggcaggaggcacggggtggcgtggggtcgcatggctgcaggcttcggcgttcagtgattgtcgctgggcacaggggcggcgctggtgcccacggcagcctgcaccttctccaccagcccggcccactggcgctgcatgtcttccaccaggggctcgaaccagctcttgaggcgggcctggaaggcc

SEQ ID NO: 29 - DNA, 389 bp, Homo sapiens
  misc_feature (10)..(10): a,t,g,c
cagggaattnnttcatnatggaaaaagacaactgaatgccctcaactgaatgtcttcatcccctcttgcctgaaatttccaccttcccataggctggggagggagtcagttccagagcagaggagggtgacagggttgaggagggacttgtgagagctagaacttggcaaaatggcctagcccacccttcaaaggggaaaagagggaggaacaggggatgaaaagttntccgcagccttcccttgaactctcccctgctgggggagggaggaggttaaagcaagaccccctgcccaggtggggagagctgggggccaggggagaaggggacaaatggtagggacacattctgtttgagcacaatgctaaaaattctgtacatcctttgg

SEQ ID NO: 30 - DNA, 157 bp, Homo sapiens
  misc_feature (1)..(1): a,t,g,c
nattcggcaacgnggaaaggaaagaaactaaccaacaaaagagaaaaccaaaaataatcacaacagaaaccagctgccccaaaggaggccagtngtngggacgcagagggtcctcagagcaggagtnacaagggaggaaagaccaaaaaaacaacca

SEQ ID NO: 31 - DNA, 329 bp, Homo sapiens
ttttttttttttttcaccttacaaatactccatgttttactagatgtgagcaaatcattaagcagcaagtttagtttggcgacaaaattgtaacatctactacaatatatcttcaaaagaaatcattcacaaccacactcacatgacaagaagacctcacagactcaaaataaataggaaaaactcatacataaatactgtcccgttccaacactgagactctcagtcatgcagaaaacaaattgaggcattgagtggaggcaaagggcactttacattatcttcttgaccctctgttttctctactggaaaatgaggataaattctag

SEQ ID NO: 32 - DNA, 466 bp, Homo sapiens
  misc_feature (2)..(2): a,t,g,c
anattgatcaaggttttcattgcttttattcaatgaggtttggaacatgagaggccaaaaatgaggagcacattttggggaatcctctataaagaatataagttaagaaagttgtccaagatgaaaataataggaaattaccccaactcatgccagttgggtcttaagaaatgtccttgatggcaggcttgaagaaaagatgtcacagattagcgtcttcccagagtgtgaatagggcacgttctcctacatgcaaacacacatgcgcacacacacaatacccttacacacataccccagcttctatgaggtaaaacttgctgttgctgactctcttctctcttaacctcttgttgcatttctccggggacagctgggagctgggttctcattaacactgaactctttaaccggggcatcccggttgggcttgcgcaagttctgggggtgtgtcatacaggaaggg

SEQ ID NO: 33 - DNA, 469 bp, Homo sapiens
  misc_feature (2)..(2): a,t,g,c
tnaganccagtccatcgccatcgagctctctgacctggttgtctactgcaaaccaaccagcaaaaccaaggacaacttagaaaatcctgacttccgagaaatccgctcctttgtggagacgaaggctgacagcatcatcagacagaagcccgtcgacctcctgaagtacaatcaaaagggcctgacccgcgtctacccaaagggacaaagagttgactcttcaaactacgaccccttccgcctctggctgtgcggttctcagatggtggcactcaatttccagacggcagataagtacatgcagatgaatcacggcattgttttctcttcaatggggcgcacgggctacgttcttgcagccttgaggagcatggagggacagaggaaatatggaccccgatggccacccgagttnccagagggaaggttccttgatggacggttgacagttcaaggtttttcggtgctt

SEQ ID NO: 34 - DNA, 272 bp, Homo sapiens
  misc_feature (99)..(99): a,t,g,c
aacgcacgttttattggaagtcttggcggcagggggagtctgcgggggcagggctggggaaggggcggcggagggggcggtgggcgcgcaggtggagcntggganatntcaggtgccaggggantcctggccngnttccatcgntccaggtttttctaccncctgcggntggacanacggcggatggagctgcggaaanttccctcctcttcatcggnttccccagtcctctgctgctggttgaacttgcaccggcatnttctgntcagcac

SEQ ID NO: 35 - DNA, 448 bp, Homo sapiens
  misc_feature (28)..(28): a,t,g,c
cctttcccacaggggcagcctgtacgantngtggcagctgggcctcaccccggcangcttgtgcgtgaccccctgagtgggggaaggcaggctgttgcatggtggcctgagcgagcagaattcctccagggacaatggcgtctcttggccacatcttggttttctgtgtgggtctcctcaccatggccaaggcagaaagtccaaaggaacacgacccgttcacttacgactaccagtccctgcagatcggaggcctcgtcatcgccgggatcctcttcatcctggggcatcctcatcgtgctgagcagaagatgccggtgcaagttcaaccagcagcagaggactggggaacccgatggaagaggagggaactttncgcagcttccattccgccgtctgttccacccgcaggcggttagaaacaactgggagcgatnggatttcngcc

SEQ ID NO: 36 - DNA, 423 bp, Homo sapiens
  misc_feature (186)..(186): a,t,g,c
cacaaacatccagagtttattgctcttcaccatggaaaaaaagtctcttcccgatgggtatttacaaagcggggagatggagtgtgaaattaaccagtgttgaaatcgtaccacccattcacatcttcaggacagtggcaggaacagcgccgtatgcttgagggtgctgtggtcacagctcgcggnccccggccaccgaggcccagttcccttccagtagtgggcagctcagtgaggctggatagaaagtctgggcaggcagtgggtaagcaattgaagcaagaagagaaaggagattttcagagaaccaaaggcgcccttttgaaggttccatctntctcacagggaggtttntgaccnacagctntggggcttccagattntacccgcactcccattttcagctttggggccagtctgggt

SEQ ID NO: 37 - DNA, 359 bp, Homo sapiens
  misc_feature (317)..(317): a,t,g,c
agaagcagttgtgttaatatttagaagaagatgtatatcttccagattttgttatatatttggcataaaatacggcttacgttgcttaagattctcagggataaacttccttttgctaaatgcattctttctgcttttagaaatgtagacataaacactccccggagcccactcaccttttttctttttcttttttttttttttaactttattccttgagggaagcattgtttttggagagattttctttctgtacttcgttttacttttctttttttttaacttttactctctcgaagaagaggaccttcccacanccacgnaggtgggttttnaggcaaggggaaggtagccgggga

SEQ ID NO: 38 - DNA, 633 bp, Homo sapiens
  misc_feature (606)..(606): a,t,g,c
ccttctccatcatttctatatattttggggatttttgttttgttttgttttttaaaaaaagatacaaacaatcttcaaagtttacctttttggacttaaggcataacatgaaattgcgcattattctaaagcttaattttgaaatgaactagtttaagtgctatcaaaccctgtttgcgtttacattatttaaattatatagtattttatttctgttcttgctgtaaattctaatgctgttcatggattgtgcaattcctatgcaatcggtctttgcctgctgagagttattaacagtgcagtgtggaatccagagtgagctttcattttctcagttatcttttcagttcaatgcatgctgtttaattgtgtggaagatccaatccatttttgttgtccagccaccatgatgtgcatcattcatttgtttcatgaaatactccaaagcctcttgctcagttttatctaaggctagggtctttcgaatgtatgcaatgtcatcaaaagattgtagttctggcattccagagccaagcatcattgagaaaagatttatgaagagatttgcatgctgtcgaatagctagattagccttgtacacatctnctgaaacctttcaaattctcttggctt

SEQ ID NO: 39 - DNA, 414 bp, Homo sapiens
aaaatcatgaactttttattgaaagtttttttgttctgaaaacagcagctttgtcatattatgacagatgtgttttttattgctgcaaaatagttaatgtagttaaatataagcacttagaggagcaatgcctggcacacagtgaatgttacatattagctgagctgttactgttattccttaataattaagttctgataattattcagcctgaaaattaaaaaaaaattagcacaaggctttgtaggtaagaccattatagatctttctaaatatttaaggtgtgttttgtgtcaccattaggtgtagatggtcagccttttgaacaaactgacactacagaagaggcaggtttcagctatctaaaaatgggcaactgttaaaaagcagttgggattgctacgttagggtggt

SEQ ID NO: 40 - DNA, 442 bp, Homo sapiens
  misc_feature (8)..(8): a,t,g,c
atcttcancaatggcaaaactttcttccaataatgaacacttctccctatgtattctcgttgatcataaaaagaattaagaccgctgccctaaaaaagaaagttaaaagcacacaactttaaggaacacgcatgattaagatctaaagttcatttacaagttggtaagnacctgggctaaaatttcaagtcaaaaccaaatgggtacctcaatagtcattngttcaacttccaaatactgtaataactaaaccaccttaataccagtgaactcactgcaacctctgccgtgcaggnttcgggcgattctcctgcgctaagcttcccaagttggctgggattgcanggcacctggcaccatgtctgggctaattttggtagttttaagtagagttttttttggaatttngggtaaaagncggggtttcaccgggttgggccag

SEQ ID NO: 41 - DNA, 432 bp, Homo sapiens
cctttaacaagcatttattgagtgcctactgtgggcttacaatgcagggcttgggatccggggctccattgagctaggtgacagggtggctgccttgtcacagctcatagtgcagaggtgggaaggcagtgtcctcgtcgtcaccctcacagctcagctctagcaccaacacccgctgcccaggagcaggtgcctggcctgtcagctgctgaaccagttctgtcaccctgaggggcaggtgctgggcctgcttttcaggtgaccatccggccgcatagagcagggctgagccgtgcagcaggatcctcaccctcaacccgtgctgctcctgaagatgaagccagcaagcgacttccaagggttcatctcaaggctggcccaagctgggtacctttcagacgggtcccaagaaggtccacttcaggtgatgga

SEQ ID NO: 42 - DNA, 417 bp, Homo sapiens
  misc_feature (61)..(61): a,t,g,c
ctcccgctttcattatggcatcaaacagctgctgaggcacttcccacctaataaagtgctngaggatggaactcccttctggtcaggtcccaaacagtgtccccagcccttggagtttgacaccaaccaagacacacacctcctctacgtactggcagctgcaagcctgtatgcccagatgcatgggctgcctggctcacaggactggactgcactcagggagctgctgaagctgctgccacagcctgacccccaacagatggcccccatctttgctnagttaatnctagagctggcttcggcttctgctggagtttggccctgtagcagcagaaggaantgaacaaagccntggaagtctggagtgtggggccttcccctggaagnctttgnatgtttgagaaggatgatgncttg

SEQ ID NO: 43 - DNA, 450 bp, Homo sapiens
tttttttttttttttttaacttgtttagatagtatatttttctagatcaaagaaaatgtagaaaatcagactcagcacatgccagttcatctgtgttttgaagctgaagcaggctggatttctgagaagcacagagatgccgactttcgttccaagttttctgttcactggaaatgaagtaacagttgcaccggtacccaacccatttttcttggcaagaacagcagtcagagtctttctggagttctatgttgggtcctggagtaaatgctggctcaatactcagtttagtaaaagctgccatgagaaattatgttccaagagcgaagtagagaaggcacgatgtgtactttttcagaaagaacagcgttgaattgttgaagctgagctggagattaaagaatctgaaagttgagtatgaagaaatttagcaaaaaaaacctcgtgccg

SEQ ID NO: 44 - DNA, 476 bp, Homo sapiens
aggaattaagcaaccacatttttaatcaataaatatgaacatttgttttttaaccagttactattaggcagaaaaaaacagtcatttatcaagaattcaaaagataacaccatcttgaaaacaaagtgccaaaaatctttgccataaactcttcctcacttatagaactaaaagtaacaaatatttactcaaaataaggcttggaataatctaaaaagttccagatatattttaaagctataacagattttgcacataaagccaaaacagattgttttacacatacatagactatctgacaacccaatgaataaatgattgaatggataaattgttgagcattagtgattacttatgttcacattatgggccaggcttacatccatttcttgttcttctcctggcctcactggtcccttgctcttccaaggtccttgggtgaggtggcttccagataggcctgggcagtaaggtgc

SEQ ID NO: 45 - DNA, 258 bp, Homo sapiens
  misc_feature (173)..(173): a,t,g,c
ttttagtagactgaccatgtcagaccctgtcaccaatgctcagctctgtaaacaatggttatttccatgttctgggggaattgttaataataggggcactagactcagcctagaaagttctagcacatcactatatacagtcctcaaaaataatttatcgtactgtttctaanaaaaaaggtactaggggttgttcttttgctctgtttccatattctggagctgcacgtgagcacttttggcttacaagtccaaaaa

SEQ ID NO: 46 - DNA, 612 bp, Homo sapiens
tttttttttttttttttccataccattcatgctttaatgaacacatatacatgaaaataagatggagagttctagtggttttgcccttcctcttcaacggcgaaattgctatttagaagtgggtagcgctcgctctctgatgggactgagctggcatagagacaaggttgccactgccccgtgtcctgtgatgtgacttcagagcttccaaaacgcaggcaagcacaacggatgtctcctgggcgaccatttagcacctttgatttcacttgggcttcatgacttctgttgtctgttcccggcttcttaccaagaaattcttgttcttttggttttctagattgttcttctactcttcctctgtctccgctgccaggtgagcccactcaggaggaggacgctgatcagcaggaaaacacagccggccactgctacctggtactctgttgtaaatatggacgtctccatggttgcagccatttctgtctgtatttgaaaatctgttgttgtgacgaggcaggaagtctcactctcaagacggccttcggggcttgcctgaggcttctcttcacctggaaactgactggtctccatttcacctgtgcatatgag

SEQ ID NO: 47 - DNA, 499 bp, Homo sapiens
  misc_feature (353)..(353): a,t,g,c
cacgagggccagcccaatacttaaagagagcaactcctgactccgatagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgcccacgttcatcatggtgcctggctgccaggcagagctctgtgacgatgacccgccagagatcccacacgccacattcatcatggtgcctggctgccaggcagagctctgtgacgatnacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcc

SEQ ID NO: 48 - DNA, 434 bp, Homo sapiens
  misc_feature (68)..(68): a,t,g,c
tttttgagtgggatctcactttaatggagaggacgttatgacctccggggcatggctcttggcttggnaagcccctggttttcatggacctgtcgatccacttgaggaaggcggtgaccttggtgtagatcccgtacttccccttacgggcacagctnctccccagctgacgatgcctgtcacgaataggtgtccttgaagcgggtgacgtgcgggcccccntngtccccctggcaggcatcctcctgcttggtgtcgtagccggcacagaacatgttctgggtgatgatgaagctgctggacanttgcagtgtttgcngtccacgtagggcacctccagcatcttgaagcctggtggactgccggcccttctcgtgggtgcgcccgaagccgctcacaatccccgtcttctgcgtcatcagcgtggantcggg

SEQ ID NO: 49 - DNA, 612 bp, Homo sapiens
  misc_feature (5)..(5): a,t,g,c
agcangttgtnggcggtgaggcggtgcacgaggtggaggtggtcatcaagcacaaccggttcacaaaggagacctatgacttcgacatcgccgtgctccggctcaagacccccatcaccttccgcatgaacgtggcgcctgcctgccctccccgagcgtgactngggccgaagtccacgctgatgacgcagaagacggggattgtgagcggcttcggggcgaacccacgagaaggggccggcaagtccaccaggctcaagatgctggaaggtgccctacgtggaccgcaacaagctgcaagttttcccaagcagctttaatcattaacccagaacaatgttcttgttgccgggttacgacaaccaagcaagganggatgcctgccaagggggaaaagccggggggcccgaacgtcaacccgnttcaaggacacctaatttcgtgaaagggaatcgtnagctggggaaaagggctttccntaaaaggggaaagtacgggattttaaaccaaggttaccgcnttccttaaagtggattganaggtccttgaaaaccaggggntttccaaagggccaanagccattcccngggggtcataaaagtcttttccctt

SEQ ID NO: 50 - DNA, 225 bp, Homo sapiens
  misc_feature (134)..(134): a,t,g,c
tgaaatgcaaccaggcccaacacacaattttttattggtttagcctatttcgtacttgggggttgggagctcaatcttcagggaaaaaaaaaaaaaagagaagtattcatgtaagtaggagatgagaagcagantgagaatcacttttaggaacacagattgggagcaggtacaggagaatcctgggtgaggatgaagaatgacctgggatggttttggagctag

SEQ ID NO: 51 - DNA, 420 bp, Homo sapiens
  misc_feature (1)..(1): a,t,g,c
ngtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcngtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaaggacaggggcagatacgcagtattttgggcccaggcacccggctgacagtgctcgangacctg

SEQ ID NO: 52 - DNA, 335 bp, Homo sapiens
  misc_feature (4)..(4): a,t,g,c
gcgnccgcgctgctcctgctgctgctcggcgctngtacaccgcgcgtgtcggacgggtccaaatgcaagtgctcccggaagacnccnaagatccgctacagcacgtgaagaagctggaaatgaagccaaagtacccgcactgcgaggagaagatggttatcatcaccaccaagagcgtgtccaggtaccgaggtcaggagcactgcctgcaccccaagctgcaganaccaagcgcttcatcaagtggtacaacgcctggaacgagaagcgcagggtctacgaagaatacgggtgaaaaacctcagaagggaaaactccaaaccagttgggagact

SEQ ID NO: 53 - DNA, 306 bp, Homo sapiens
  misc_feature (87)..(87): a,t,g,c
tttttttaatctgcaaagtcctttgcacaagtctcccaactggtttggagttttcccttctgaggtttttcaccctattcttcgtanaccctgcgcttctcgttccaggcgttgtaccacttgatgaagcgcttggtgctctgcagcttggggtgcaggcagtnctcctgacctcggtacctggacacgctcttggtggtgatgataaccatcttctcctcgcagtgcgggttactttggcttcatttccaagcttcttcacgtcgctgtagngaatcttggntcccttccgggaagcacttgcaa

SEQ ID NO: 54 - DNA, 430 bp, Homo sapiens
  misc_feature (73)..(73): a,t,g,c
cctcaactaaactctttatttttggtttggtttttaagtaggccatggttgctttgaggccactgtatggtgnnaaactatagatgaaagagcttttattaaaaaaggcactgatgggagacactggctgcccccgaacctttgccctccctaagagcagcctctcagttcccttttgaggagccccctcacctccaccagatatgggaagacaaatggggtccctgtttctggattcccagcatggtcgggggaagatctgtgacctgggcttggcacgggggctctcagaaggcaaggcctggttggagggtgttggtggggggcagaaagctgagcaggattctggggangcagcccaccccatatcctagtaactcccttttggctcttccagacttattatgcggcttggaaccatttgatgcct

SEQ ID NO: 55 - DNA, 410 bp, Homo sapiens
  misc_feature (309)..(309): a,t,g,c
cgaaaacagactcctctagtacttggagatcttggacgtacacctaatcccatggggcctcggcttccttaactgcaagtgagaagaggaggtctacccaggagcctcgggtctgatcaagggagaggccaggcgcagtcgacctgcggcggctccctaagaaggtgaagcaacatgggaacacatcctaagacaggtcctttctccacgccatttgatgctgtatctcctgggagcacaggcatcaatggtccaagccgcataataagtctggaagagcaaaagggagttactaggatatggggtggnctgctcccanaatctgctcagctttctgcccccaccaacaccctnccaaccaggccttnccttcntggagagcccccgtggccaagcccangtcacagntc

SEQ ID NO: 56 - DNA, 547 bp, Homo sapiens
  misc_feature (409)..(409): a,t,g,c
gtcttcacttttattgaaatacaaaatgttaaatatgcaagctgtactaatgaaggtgctccttgaagttgattaaggagggctgggctgcttgtggcttccattttcaattgccaggaaagaggtagaaatatcttgtcatggacagtcgttctatggtgggcatttgagctttggcccttggagtttcaaatgattgctgtaccttccgaaatacttcctctaggtggcagcaccaagaatatttctggaagcatgtgatgagttgtgtgatgaagatagagcccattgtgctgtctctccaggacacgttgtgtggcgttgaagagcagaaagcaatgaagtccttctccacgtgggtcttgtaaacagcatcttcctccaggttctcagatgactgtgaagaggncacttccaaggatgccggagagtctctgaccccacagtttccccacgggtttggaccnctgcaggcctggacnatgatgaccctgggggttgtccctcaaactgaggcagttccggttgttgaaaacncggaaaangt

SEQ ID NO: 57 - DNA, 406 bp, Homo sapiens
accaaatatcccccaataaaaaagctcatccgaatatggaggctggaccacctgagtcaggagaatctacagatgccctcaagctttgtcctcatgaagaattcctgagactatgtaaagaaagagctgaagagatctatccaataaaggagagaaacaaccgcacacgcctggctctcatcatatgcaatacagagtttgaccatctgcctccgaggaatggagctgactttgacatcacagggatgaaggagctacttgacgggtctggactatagtgtagatgtagaagagaatctgacagccagggatatggagtcagcgctgagggcatttgctaccagaccagagcacaagtcctctgacagcacattcttgggtactcatgtctcatggcatcctggga

SEQ ID NO: 58 - DNA, 180 bp, Homo sapiens
  misc_feature (177)..(177): a,t,g,c
gtgtgcaggaaccagcaatgagaaggccaggaaaagaaagagctgaaaatgcagaaagccgaagagttagaacttttggatacagcagaagaaacagcggctccactaccgacctgcccccggttcgatgtccttccaagaatgaagtctttccctggtgatggtcccctggcctgnctt

SEQ ID NO: 59 - DNA, 421 bp, Homo sapiens
caacatacgtttttattactcaaggacaacctggacgtcaccaatgcccagcttcacgggggcatgtagtgtgactcacggctgaacacaaaatcactgtgaagcctgtgctacagccctgggctacttttggacacgcaagaggacactcggttacatgaaacctgacttgcttctgaagacatgtgatacatgtcttttccctgaaaatgtttcccttccctgtctgccatgctcccaacttttctgccttccaggggcttctcactgtctgggtaactaggtcttgggcctagctagaccttgggtagtggcccctctgccacaatcagtgcctgggcctgaggctgagcttgtggcctccgaagcctgaactggctcggagctcgtctgtggcgcccagggatggcctggcttgcag

SEQ ID NO: 60 - DNA, 458 bp, Homo sapiens
  misc_feature (414)..(414): a,t,g,c
ttttttttttaaaatgccaagataagaaacgatttattatagagagaagaaaaatttctcatccaaaatatagaaatctgtacaactttgccacaatcaatatacatgaactgtacaaatttacaccagttcataatttaccaaataaaagatgactaacaaagttcacaaaatagatggtggtttgtggaaaagacttttacccaattaagtacaaggaaagttacaaaccagacctccactttctaaaaataagaagtttactcagtcttagaaaactacaagctagcaaatgtacagagagctggctggtgctaacaccacagttgagacagtgtctttttaagggtcttttttaaagcctgttgccatggcagattctggtcacttgctactttcaaggccaaaaacacnataccagggtctgaccatttccccaggtcatgcttactagtttt

SEQ ID NO: 61 - DNA, 263 bp, Homo sapiens
  misc_feature (10)..(10): a,t,g,c
cctcgcctanctnttatttgctagggagggaagtcctagggtggtttcagtttctcccagacatacctaaatttttacatcaatccttttaaagaaaatctgtatttcaaagaatctttctctgcagtaaatctcgcaggggaatttgcactattacacttgaaagttgttattgttaaccttttcggcagcttttaataggaaagttaaacgttttaaacatggtagtactggaaattttaccanacttttanctagcnctt

SEQ ID NO: 62 - DNA, 420 bp, Homo sapiens
gtatttttactagggacagcgtttaccatgttggccaggctggtctcgaactcctgacctgaagtgatctgccctccttggcctcccaaagtgctgggattacaggtgtgagccactacgcccagccttttggtttcttatgtgtaggagaggataagtttctttctcagagtgtgctgcagccagacataatatttctccttggcagaaactctgctgttccaatcatacatgtagccattgagttcaatgtgaagatccaggagtgggcgttttctttcttgtctcagtttctgggagaggcactgcaggtacagggatggtgagctgtgagactgctccagcaggggacatgtccgcagtacacaggctccagaagaagtcagcttctcggtgaactgtgcacagccctcgcttctg

SEQ ID NO: 63 - DNA, 615 bp, Homo sapiens
  misc_feature (120)..(120): a,t,g,c
acaagttacaggaatgttctccaagcagcaatccaaaagagtctcaaggatccttcaaataacttcaggctccataatgggagaagtaaagaacaaagacttaaggaacagcttggcgcntcaacaagaaccagtgaagaaatccattcaggaatcagaagcttttttgcctcagagcatacctgaagagagatacaagatgaagagcaagcccctaggaatctgcctgataatcgattgcattggcaatgagacagagcttcttcgagacaccttcacttccctgggctactgaagtccagaaattcttgcatctcagtatgcatggtatatcccagattcttgggccaatttgcctgtatgcccgagcaccgagactacgacagctttgtgtgtgtcctggtgagccgagganntcccagagtgtgtatggtgtggatcagactcactcagggctcncctgcatcacatcaggaggatgttcatgggagattcatgcccttatctagcagggaagccaaagatgtnttttattcagaactatgtggtgtcagagggccagctggaggacagcagcctctttggaggtggatggggccancgatnaagaatgtg

SEQ ID NO: 64 - DNA, 660 bp, Homo sapiens
  misc_feature (434)..(434): a,t,g,c
gacgttcaccaacattgttctcctgtcaccaggacaggacgcggcattcacaacggaagaaaacggagcatcgtgtggcgggaagggatcatgcgggtgggaacatcgggaggcctaggctcaggatcccggcacaccacagcggtgctggaggagaagaggcctcgagcgagggcaggggacggcaccgagagttgccaggaggtggctcaccgcccagtgccccgggggccctccaggtcgggccgggctgtggagaaggggtgtaagcggtcacgaccccgctccttgcagcctctggcctccttatgtctggagggctcccatttgattcagcccaaccctgcccgctagcgtctggggataagggaagactcggcccgcctcgacagaaaatggggtctcagcactcgctgggtgacccgcgggagagngcaaaggagggctcccaagtccgttctgcagcactggggcagggaacagacccaggatcctgggaatcctcttctgcctagctttgcctgcctgccagagcagggcctgcgatttgggtgctgtgaccgtccgggggcgcgggaagggcaagggaggcggatctctgaagttccgcccaacttcgctcctgatacccaaggtcagagagggccagctgggggcggg

SEQ ID NO: 65 - DNA, 510 bp, Homo sapiens
  misc_feature (224)..(224): a,t,g,c
tttttttttctcaggaaactttttaatgaaagtgtcggccacggtggtgtgtaggtggctgagctcagattgcagctgctaagacaccagccacttaccaagagaaagccaggctgcttcaaacccagggcccaaggcaaaaaagcatcacttccggccggggagtctggaagccacgccttgtgggaggtcacactggcatctaggccttcgcctgcattgcngaaggagagccaggtccccctcctggnagaacgctgcgttccccagccccacaccggctttgccaccacacaggctgttgaggcaggaggtgggtaagacgtagctgtagacccaaaggcaaccaccagccctggggaccctgcgggagnagggagcantttttaggaacatgggaaaagtgtggttcattcccttctttaggacagcacacttcctnacttaattaaaaattcttttggggggaaggaggtttgggggagggattaaaaaatttgggcacagttt

SEQ ID NO: 66 - DNA, 463 bp, Homo sapiens
  misc_feature (91)..(91): a,t,g,c
cttgctgatgacttgttttaaaactttcatcctaaataaccttttgatacttgaatatttgtaagttttatacatagtttctaattttttnccgaacagatccagatacctaataagatgctggaatgtaatccctggacaatccgtgtcctggcagcatttggtcttcctctaagcgcctggctccgctgttctcaggagtgggttctgaagtctctggagaacaggatacgtggagggttaggaaggggccaggcctagagacgggagactccctcccggagcaggtggaggcacaggaccgttcgctaccccatctgccggcacctgcgggggagcccagggcattntttgttaaaccctcctggaccacctgggttcaaaggaaaacagaagcatgggagggccgccaatttttttcaaggaaattaaccccttgaatatttccttcatttttttagga

SEQ ID NO: 67 - DNA, 439 bp, Homo sapiens
  misc_feature (235)..(235): a,t,g,c
tgctttaaaaatatcttcaataataagacatcctatttttggggggttagggatagagataaatatgaaaacttttttttgataaaacacctagtaaggacatgtttccaggtttaaagttcatgaacatataaaacagaaattgggtcctgccaccttgagtgcaggattctgaggtcctaaatctggttctgaggttctgtgtttgattctggggtacagacagctcaagtanggaggtctccttatgtcacaaaactacacgatgcggaggttctgttccccaccaaaggcttgtctggggcccattggtgcctggggaggaggccccagcgaggctcatgggtccagcatggctcctggggggaccccttactctcaccacgcaagagcccccagccaggagcactgggccagccagctgcgggccgatgagggn

SEQ ID NO: 68 - DNA, 189 bp, Homo sapiens
  misc_feature (32)..(32): a,t,g,c
ggttgtcggcagggctgtcagggcacatgctnngccggctggggtccccacccgcggagacggtcctagacccagtggggagtgctgtgtctcctgggtctgcccacccgtggccaagcaggaactccaggtgcagggcgggcctccaggtgcagggcgggccagaagccccctcatcggcccgcagct

SEQ ID NO: 69 - DNA, 467 bp, Homo sapiens
  misc_feature (399)..(399): a,t,g,c
ttacactaaaaagagtaaattttattgtttataaattatgcttcaataaacctgcctttaaaaaattttttatcagtaattacaattttcaaaagtggagatacttggtaggcccctgctttcttggcccctaaggtccctctctctgttaaactgtagagctccttagaaccaaaaccaaaccacctttcattttaatgaaaatgaaatgcaaaaactggggctccaagcaaggaagaaaccgtctacctgcagttcatctgggggttggtgggagaggtggtggagagagtaaggctaagcgcccccagcaggcccaagttctctgtaccacacttggaggcaggtatatgggactcgaaagtggatgttctcagccacaagcctgggcctggatangaaacaaaggataagactggaaggatgtcaggtaaggggagacccagagcancagctgggctcacttt

SEQ ID NO: 70 - DNA, 226 bp, Homo sapiens
  misc_feature (210)..(210): a,t,g,c
cattctgggaaatcaggctgtctgtgtaaaggttactgctgtgctcaggagttgaaccgtgtggtgctgtatccggatactcatgacgaatggatggagggcgtgaaaagtgagcccagctggtgctctgggtctaccctacctgacatccttccagtcttatcctttgtttcctatccaggcccaggcttgtggcttgagaacatccantttcattnccatatac

SEQ ID NO: 71 - DNA, 519 bp, Homo sapiens
  misc_feature (372)..(372): a,t,g,c
tttttacaatgtgtttattggacacacaaaaaaactttgcaaccatcataatacatcaatatttaacctagataattctgaaataatttggattctttcatttttcaggatttgagctcatcaattatgttaaatttcctatattctgttacaaatataatacagatttcataagtctgccttgattcactgctccctaatccagagcagttaaatttcaaaatcacatagttatatagcctacctgccaaaaaagaataacccatccctgtcttcttaaagtggtagggttttaaaattcatgatacaagtaacaaagtaagttgataacaaatactattttgaactggggcaaaatctagcccaatatgnaattgtgttccccttgtatctgagaggcctgtgttggacaaccatagtactctgctttcactgtgctcaaataacctgaaatatntagttgtttctgagtagntctgagtaggaaagaaccnaatggtaaatttccttagagaccat

SEQ ID NO: 72 - DNA, 254 bp, Homo sapiens
  misc_feature (8)..(8): a,t,g,c
ctgtggcngagttttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgaaatttgcgaagnatttgagtagtgaaatgtaagcacaaaacctcctgaacccagagtgtgtatacacaggaatnaaccttat

SEQ ID NO: 73 - DNA, 693 bp, Homo sapiens
  misc_feature (38)..(38): a,t,g,c
ggtgttgaaggaatttattcatgatagactgagggtcnatcagggngactgtccaatggtgacaagctccagaagcccgcgtcgcaacagccaggagggccaggncaccccaggcaggaggcagtgggctggcagccaccctgggcacagaagagcagacgcagacagtgctgggcaacgaggggctttcttcatgggcccgcctgccctgtccctccccccaggtccccaccttctagggttaaagtgcagctgggagggaggaggcaggcagaattggggagctagagagagcccaagtgaaccctgactgtccacgcaagtcccatgtcctcctcgtcctggagttcctcgaggttcagcgagcccatcccgcctagggcctctggaaccttggggcgagggaggtaacccctcactcccacacccatccgatatttacnaagagcatccctgggagaggggagaaggcggccngctctgacagctttctggggggccagaacttggggcaagtgcggggntaacattctaacaggagagaaggggatgctctcttcaggatggtcttgggggttgtccctccttctggccttgaaactcttccttcctttttcctctcccttnccaacagnaagcaagaancagaaagccgaaactgtccatccggcacattnagttccagtgatcaaa

SEQ ID NO: 74 - DNA, 175 bp, Homo sapiens
aaggcctctcaccccacttcccatctccagggaagcgtcgccccagtggcactgaagtggccctccctcagcggaggggtttgggagtcaggcctgggcaggaccctgctgactcgtggcgcgggactgggagccaggctctccgggcctttctctggcttccttggcttgcctg

SEQ ID NO: 75 - DNA, 485 bp, Homo sapiens
  misc_feature (467)..(467): a,t,g,c
tgaaaggctattgcactacaaagtaaaccctgaattcaatatgaatccccctgtaatggaccagttcaatatgaacttagagcaacatctcgcaccccaggaacatatgcagaatttgccctttgaacctcgcacagctgtgaagaatttcatggcctctgagttggatgataatgcaggactatcaagaagtgaaactggatcaacgatatcaatgagttctttagagagaagaaaatcacgatattcagaccttgactttgagaaggtcatgcatacaaggaagaggcatatggaactatttcaagaactaaatcagaaatttcaaactttgggacagatttcgggatataccaaatacaaggcagtatgggaaaaccccggcaccaaacaaggattccatggggacatttttcaaaaaccccgtggattacccgctttacaccacattcatgtttttggacacnggggcaagggtgnttttt

SEQ ID NO: 76 - DNA, 581 bp, Homo sapiens
  misc_feature (528)..(528): a,t,g,c
ttttttttttttttttttttttttttttttttttaaagtatcacaccagaagtttattatggaacaatcacatatgttgactctcctttgaccctcactgcagtgcactttcattacttatcaatctgggggcgggaaaaaggggtgcagccagacaagagaatatacaggaaagaagcattgtatataagcctatgtatttcagtaatgctgctacagggggatacaaaatcaggtgccagcctccagaaaaaaagagattttttttcttccctcagtctcatttggcccctcggcgcttcctgaagtagcgattataggcagcattgtatccataaaccatggcgtagtttcgcaaagtctgtagtcatcacaggcttccctattgagctcgtggacaggcttagagcgttctcggatcctctcttggactttagctctccatctctgctgaggggatatgaaggtatttgcattctcctgttaatgaagggattaagttcataagattccatgctttcatgtgantcataacacnaagttctaccgtaaggggccangatggaagaaggtcagctctc

SEQ ID NO: 77 - DNA, 541 bp, Homo sapiens
  misc_feature (14)..(14): a,t,g,c
cctactgctgctanacaagaccctgagactgacctgcaggacgaaaccatgaagagcctgatccttcttgccatcctggccgccttagcgtaggtaactttgtgttatgaatcacatgaaagcatggaatcttatgaacttaatcccttcattaacaggagaaatgcaaataccttcatatcccctcagcagagatggagagctaaagtccaagagaggatccgagaacgctctaagcctgtccacgagctcaatagggaagcctgtgatgactacagactttgcgaacgtacgccatggtttatggatacaatgctgcctataatcgctacttcaggaagcgccgaggggccaaatgagactgagggaagaaaaaaaatctctttttttctggaggctggcacctgattttgtatccccctgtagcagcattactgaaatacataggcttatatacaatgcttctttcctgtatattccttgtctggctgcaccctttttccgccccagattgataagtaatgaagtnactnagtgaggg

SEQ ID NO: 78 - DNA, 454 bp, Homo sapiens
  misc_feature (165)..(165): a,t,g,c
gcaggtaaaaattatacttttatttgagtcaccaggagaaagattcacttgtggttcaagtcaaatgttcagaatcataacaggccagaaaggtttgatcccgagcacaagcccacgagggaggggaccaaaacagaccaaaatgagacaacaaccccatataanaagatgaactggcggcttcacacactcacacacatacacatacacacggatgaaatgtttggacagaggcaaatttcacgtggtcatttctgtttctttttaaatacaggtttgtagggtggtattttgttttttccagctataaaaaaaggcccaaaagtgcatgtgtgaggggggaaaggcagaaattaagcaataaagtcattttcccctggagggacatgagaggggagaaaacagggaggcagtgcttgggagaacgcactttcctcaccactggggcttcttg

SEQ ID NO: 79 - DNA, 383 bp, Homo sapiens
aacaatcagtcatcatgggtgtttttgtcaactgcttgttaattgatttggggatgtttgccccgaatgagaggttgaggaaaagactgtgggtggggaggccctgcctgacccatcccttttcctttctggccccagctaggtggaggcaagtggaatatcttatattgggcgatttgggggctcggggaggcagagaatctcttgggagtcttgggtggcgctggtgcattctgtttcctcttgatctcaaagcacaatgtggatttggggaccaaaggcaaggggacacatccccttaagaggacctgagtttgggaagagtggtgagtggaagggaggagcagcaagaagcagcctgttttcactcagcttaattctcc

SEQ ID NO: 80 - DNA, 545 bp, Homo sapiens
  misc_feature (497)..(497): a,t,g,c
actgttgacctgtcactgtttattatttcagcactaaaactgaggagcctcaactgctggctcttcttccctttgtatttgtgtaaggagcactgcactcccataaaaggttttaaaatacaaaatgtacaagaacacacaattccaagtgctgtaaacataactgagaaccagttcctttactaaacatccattttataaaatacaaggtttcaatttgagcccatctgagccttaaagatccattctgaataccaaaaacagggcttcacagccaggcccagaagaggtctggtgataatggctggccctgggtggggatagtttacacccgggcagcagcaccacacatgaacccaaagacatgttctttttaaagctgttttcagccatgtttctctggtgcatctccagtaagcagaaggctacccattccattcctcaaccccaagagctagcacagttagagtaggaggggggtgcgtactagcacgtgnccagttgctcagtgcggcaggtagaaatgatttgcataggtccatggg

SEQ ID NO: 81 - DNA, 506 bp, Homo sapiens
  misc_feature (11)..(11): a,t,g,c
gaacaatatanacaggcattcccatcacaggttaaagaaacaagagtcatcgaagagcctgaagaaggttattgcagctttgtcaaatccaaaagcaacctctagttcaccagcacatccaaaacaaacattagaaaacaaccacccaaatccattcttgacaaatgcacttttaggtaatcaccaaccaaatggagttattcaaagtgtcattcaagaagctcctctagcacttactaccaaaactaaaatgcagagaagattaatgaaaacattgctgctgcaagtagcacccctttttcctcacctgtaaatctgagtacaagtgggagaagaacccctggcaatcagacacctgtaatgccctctgcctctcccatcctgcatagtcangggaaggaaaaagcagttagcaataatgttaacccagtaaaaacacagcatcactcccatcctgcaaatctttagtgggacaattcagagggacagattcagacanttcccag

SEQ ID NO: 82 - DNA, 476 bp, Homo sapiens
acaggtgaaccatagtctcactttatttcgaattttaggactgaacagaaagaaggttcacagatatttcttcagggaaggaaagtgggcaaatagcacaatgaggcaaaccacacaatctagacgtactttcccgggtaaacaaactgggaagcccagagctcacagaggaggagagcagagcttggggtggcagagaggtgttcattgggaaggagtcaggaacacaagagaaatgttcatgagctcatccaggatagagaggactcttggggaaggggacctccttccccatgctgtctctgccccaagcccttaatgaagcaggagagggaagtctgtccagaaactcatctggttagacatttgcatcaggaatagggagggggtggaggcagagcactttggacatgggaaatcacagttgcagtagacagtgagctctagtcactccagaagcttgacataatttgcag

SEQ ID NO: 83 - DNA, 442 bp, Homo sapiens
atgagctttcctttgatccggacgacgtaatcactgacattgagatggtggacgagggctggtggcggggacgttgccatggccactttggactcttccctgcaaattatgtcaagcttctggagtgactagagctcactgtctactgcaactgtgatttcccatgtccaaagtggctctgctccaccccctccctattcctgatgcaaatgtctaaccagatgagtttctggacagacttccctctcctgcttcattaagggcttggggcagagacagcatggggaaggaggtccccttccccaagagtcctctctatcctggatgagctcatgaacatttctcttgtgttcctgactccttcccaatgaacacctctctgccaccccaagctctgctctcctcctctgtgagctctgggcttcccagtttgtttacccgg

SEQ ID NO: 84 - DNA, 321 bp, Homo sapiens
ctggaaagagaatagaattaaagagaagaatcatctatgtatgaagcctggtctaactgataacgcagtcagattgccatttttaagatggaaaaaggggaacatgcttgccttgagcagaaaagtttttggtggggctggagctcctaaatattattttggtcatttgcccaaaaactgaagtacaccaccaatgtcccaattatgaagttgaatacagaattataaaaaaaaattgttgaaagtcaataagaataaatcttagaatttggactcctgccacatggcagaaaaaaagcatgatgttttaagacaacctgg

SEQ ID NO: 85 - DNA, 425 bp, Homo sapiens
gcatttatttgaatatcaggttttagctaactgcctaatgtaccaaaaaaaaattagcaatgtcttatgtgggatgaggggttaagttaaagttgctgatcaagtaatggccgtgtgcacaagatgataactaagaatttctggttctaaggtagtaatgccttttgggatttatatttgtttaatgttgagattctggggaccataggtgggcctgtcaactcttaacagactatagtaaaggagaggtggtggcacatgtgagataaaacttgtttatatttacattcttattagtggatagatatcacctcagtacaacactgaaattaaactgccttgttcatgctttcatgtctcaaatcaaggcctaaatgagtaaaaagatgattacagattacctaaaacccttaaccagttttggt

SEQ ID NO: 86 - DNA, 367 bp, Homo sapiens
ccttatttgggccagttcctttaggaaaacagcaaagtcccatcccaagttagggactgagatttcacagcctctcagtctgcgctctgcaggattctccgcatttccaggcaaagccaaaaaaccaaagcaaagcacagggggccctctttctacctctcctcacccagccaaatggcagaggtgccacagaggcagaggggtggtggaagtgctgcttctgaggcaggcagtggaagtgagatgcccgtggggcaggaaggaggtgcctcgccagccccgccatccaccatccggcatcaggatgggctgctcgccaacccagcactcccactgcagaggtcgcgggtgtgaggtgcatggattc

SEQ ID NO: 87 - DNA, 298 bp, Homo sapiens
aactttttaaaagtttttatttacctaaaaacataaatttatcaaacatatcaatttgctttgcagacaatttacataaaaattcatgatgtataccaacgacagcatagcattatctacctcagtttgtgaagcatcctttcctacagatctcagcaaaacgcagagccagtaggggaaattcaagctttcatttgggggcctctggggtcctacctgctggcagttctttcagcagaaagtggggtacgggggatgctgaagagggggaagtggaccccattgcctgttaagtccc

SEQ ID NO: 88 - DNA, 293 bp, Homo sapiens
gtaacaattcagatccagtgtaaacttccgttcattgctctccagtcacatgcccccacttccccacaggtgaaagtttttctgaaagtgttgggattggttaaggtctttatttgtattacgtatctccccaagtcctctgtggccagctgcatctgtctgaatggtgcgtgaaggctctcagaccttacacaccattttgtaagttatgttttacatgcccggtttttgagactgatctcgatgcaggtggatctccttgagatcctgatagcctgttacaggaatgaagg

SEQ ID NO: 89 - DNA, 419 bp, Homo sapiens
tataaagcactgtttatttttatgtattgaaaatgtccattttaaaagtcttcaaaataaaaacaagttagaaaatttgagcttgtgttaagaaggggaaggacagaaaaggaactgaaaggcctctacgggcagcagtttaattacagggcaacaggaacccatttatagagtattgtaaacaacacaactatggcgacactgctctcagattaacatggcttcatttcctttatattatagtttagtgtgtaagcatggttaccactctgcacaagcttcgttatccagagtaccgagggcccctcatcagaagggccaaatccccgaaacatgcatatgtactcatcactgtaaaatgaagacctttcaatattttcagcaatcaactaaagtattccgtaatcacaaaaatgttt

SEQ ID NO: 90 - DNA, 426 bp, Homo sapiens
ctttcattcactttattactctctcatatatgctgtgagaagttgtaatattaattggcctctttgggtgggactctaagcagtatttgcagaaatatgcttctggtccaacttgtacatccagaacaaaagggcccctctagtagctgtgtgctggattattcaggactgattaactcaagttgctcattgaatcacatcatccactttatcccagaaactagaactgtgagaaaccaggtttagaagacagtcacagaacagcacacatccaaattcagcgccattgaaagttggcctaaggctcagtgccactcacccctccctccccaaaagacaagagaaatttggccagatggggaaggtcactggaaattgaggccaaagggctgatcagaattgcctttataatatatttgatggatg

SEQ ID NO: 91 - DNA, 313 bp, Homo sapiens
accagaaacacgtgcactttattgaatgccattgtagaaaagcgtgtgaggataaagggctgatacaggactcggctccgggggcagggcgaggaatggaagttggagtacgtgggatacaggtcatgggcagactcctggcctcagtgatgcctcctgatctatccataggcctggaagatcagcactgggatgacgatgagcagaatggtcatgaggatgcccagaatcagggcccagatgttcaggcacttggcggtggaggcataggcctgggccccggtcacgtcgccaaccatcttcctgtccctag

SEQ ID NO: 92 - DNA, 331 bp, Homo sapiens
ctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcacatgaggccaggagctctgcccatgacctgtatcccacgtactccaacttccattcctcgccctgcccccggagccgagtcctgtatcagccctttatcctcacacgcttttctacaatggcattcaataa

SEQ ID NO: 93 - DNA, 505 bp, Homo sapiens
ttgtttttttttttgcatgagactcaagattctatttattcagaggcaaaattccttcttaactgtgaacctgtgaaaccaaacaagttatgtgcttccaaaatacaatagtgggacaaacataggatggatattactattccaaaaggaagaaagagaggaagagaaacagtcatgggactcaagcaagtacaaatcatagcaaaggaaacgtcctaagatcttaaggctggagaataatgcctttaagataatcatggcccactggggcagcaccacctccctgaccccgttgggtggaggacccattaaaagtggcagcatgacccccacaggcagggaaggcactgcccatgctgtgtctctctgctgtggccctgcccatggaggcaatttcctgtggagggtggggttgcactctaactgatctgctggccttctcttccttcaacgattccctgggtgttgggtccacctttagaaatttaaacagaacccctccccc

SEQ ID NO: 94 - DNA, 590 bp, Homo sapiens
ggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtaaagatgatcaaccatctcaataaaagccaggaacagagaagagattacaccagcggtaacactgccaaccgagactaaaggaaacaaacaaaaacaggacaaaatgaccaaagactgtcagatttcttagactccacaggaccaaaccatagaacaatttcactgcaaacatgcatgattctccaagacaaaagaagagagatcctaaaggcaattcagatatccccaaggctgcctctcccaccacaagcccagagtggatgggctgggggaggggtgctgttttaatttctaaaggtaggaccaacacccaggggatcagtgaaggaagagaaggccagcagatcagtgagagtgcaacccaccatcacaggaaatgcctcatggcagggcacagcagagagcacagcatgggcagtgcttccctgc

SEQ ID NO: 95 - DNA, 272 bp, Homo sapiens
catgttgggttttgattatttgtggtgctaagaagaagggaacatggttggaggcccagtgagagaaacagtgctttgaatcaaagagcagaatgatagaaactgacttcagagcaacttcttggcagcagtatccaatttggaagttgaaggtctgtcctggagccagatgctaacgaaacacagcaaatgcttttcctaaggcacaatagtcttttcagtgagctcaggaaccctgtttatgcagatcctcgttgaactttcttgctcct

SEQ ID NO: 96 - DNA, 287 bp, Homo sapiens
atcatctgtggagtgggcatcttcatgcacaggaggagcaagaaagttcaacgaggatctgcataaacagggttcctgagctcactgaaaagactattgtgccttaggaaaagcatttgctgtgtttcgttagcatctggctccaggacagaccttcaacttccaaattggatactgctgccaagaagttgctctgaagtcagtttctatcattctgctctttgattcaaagcactgtttctctcactgggcctccaaccatgttcccttcttcttagcaccacaaa

SEQ ID NO: 97 - DNA, 133 bp, Homo sapiens
tttttttttcaactttttgcaaagcagcatagcaacaatcgtgattgtagcacttgcctgaggttgtggtcacaaccaacgtagtaaacatcatttgcatatcagtaagaaaaagaaaacaggaggagatgag

SEQ ID NO: 98 - DNA, 425 bp, Homo sapiens
gctcaatggcggtctggccgtgaagaccaatgagatctctgtgaatccagagtggggtaggttcctaccctcccggtttccttgggggccacctccgttgtgaataacgccactgtctccaagatggatggctcccagtcgggtatcagtgcagatgtggaaaaaccaagtgctactgacggcgttcccaaacaccagtttcctcacttcctggaagaaaacaagattgcggtcactaagggagaacttgcgtggaaggagcaatgcagacacagtgaaatctctagaatctgctttgttttgtaagaactcatctcctcctgttttctttttcttactgatatgcaaatgatgtttactacgttggttgtgaccacaacctcaggcaagtgctacaatcacgattgttgctatgctgctttgca

SEQ ID NO: 99 - DNA, 468 bp, Homo sapiens
tttttttttttttttgctgctaaatttgcggtagtttattactgaagcaatggaaagccaatacagacagaatatacagagagaaagagaatgagttctttcctgataatttgtaaatatttgggtcttcactggacaagcttcacagaggattcactggttccctagcaaaccagcatgtccagtcctgcagcctccctttcttaggcccagcatatgtcagctgtgtgcatagaaaaatcaaagcaggaccctgagtagttggaaagaaaagatggttggaaatgggttgcacttcaagtgaggaaacaagaggtaggagaccggcatctctttctcatatgtcccaggctgactcttgtgagttgttttcccttggaggctatcgatgacagtcacagtaacctgatggaactaactccaagatgcctggtctcaacttgacaaaaataccccaagttgggaaat

SEQ ID NO: 100 - DNA, 232 bp, Homo sapiens
tccgtctttattaacgaaagtgtttactgcctaagccagacactgagagccaccatgtattcctctctctctctcacctattcccatgtttaatccattatttggattggtccattttatctccattttctcaaatacaggaaaagacagatcatcatcaaccagcaattgtgactatctacatattcacataaggatttccaacttggggtatttttgcaagttgaaccag

SEQ ID NO: 101 - DNA, 302 bp, Homo sapiens
acggattataaaagttatatttattcacgatgctacatttattgcattcccttagaaaaatggagaactgtttatgtacccaatctgcacatataaaattttatacaaattatgtgtagcacataaaggcctctggtacagctaaaatcctgacactataatttgggtattcctgctttagggtctccagtttatcaggtctgtccatagaaaacagaaactggaattatagtcagtcttgctaacacttagaaactactttaaaatacaataaaattttcatttaccctaaaagtccaaat

SEQ ID NO: 102 - DNA, 412 bp, Homo sapiens
ctagaagaaaaagagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattactaagtgtttcatttgatttatttttctattgtagttccatttgtgaagattgtgactttttgtt

SEQ ID NO: 103 - DNA, 388 bp, Homo sapiens
ttttttttttttttttttttttttttttttaatgaactaacatcttttctttattcctataacaatttgttggaaattgtaattactgaagatacagttgtcagttcataggtagtaaaagtacgtaaccccagaatttggggaaaaacaaatatttttcgtctgaaattaactactaaaactcaactcataatgatggggactagtaatgcccagagtaaccttctgggactgaaagttctagtatttcctgaatcttggtcaagactccacatgaagtaattgcaaaggtatggcatctagtaaaatggagaatcaaagaaacatagtttttttttctgcacagtcaagagaaaagcatgctttcttctttctttcactggaaata

SEQ ID NO: 104 - DNA, 270 bp, Homo sapiens
gcagtttgcagtgatgtggctaagatacccttgtgctattaaaggcacatttacgtatctttcaacgcagtttgtacctttgtttagggttctattgaactttctctgtgctctttagaaaaccgtcgaataaagaactcaaatgatgctgtttttggacagggaggtcatggaccaagtacaaagcggcaaaaggatggaattaaggtacctgacattgttgaacccatgcctgatgccatattagctcaggactacctacatggacat

SEQ ID NO: 105 - DNA, 389 bp, Homo sapiens
  misc_feature (173)..(173): a,t,g,c
taaaacaaacaggtttattgataagcaggaatccggtccagcatacagtgcaaactttcattgtcttgggatcaacacacacacacaagggaaccaaggccccacagagtctccaaattcatcgcttcccggctctgcctgtgatgccaaggaagcctgaggttctcccaggnctgtccaccagggggcaggtgcgctccatcatgtcctcatgtgggtccaatgtgggcagaaacctgggccagtcccagcaggtctgactttttttttcttttaagagacagggtctcactctgttgctcaggctgggagtgtagtggcgcgatcatagctcgctttgcagcctcaaactcctgggggntccagtgantccttcccttcaagccttc

SEQ ID NO: 106 - DNA, 381 bp, Homo sapiens
  misc_feature (97)..(97): a,t,g,c
cttttcagggaatttcaggaactagagatgactgagtcctcgtagccatctctctactcctacctcagcctagaccctcctcctcccccagaggtgnggnttcctcttccccactccccaccttcaattcctgggccccaaacgggctgccctgccactttggtacatggccagtgtgatcccaagtgccagtcttgtgtctgcgtctgtgttgcgtgtcgtggggtgtgtgtagccaaggtcggtaagttgaatggcctgcctttgaagccacttgaagctggggattcctccccattaggagtcagcctttcccccttcccaggggccaggggctgncaagagggggaaaaccagtgttaggcctttgcccggatttct

SEQ ID NO: 107 - DNA, 240 bp, Homo sapiens
atcttttccgcccagctttttcttattacaatacaactacaatgaacatggcagattatttacttaggacaaacttataggggtagaaatgttggtgaaaaggcaaatacatttttaggcttttgatacctactgtgaaactacactaaagaaagattataggagtgtataggttaccagcagtgtaggagtggtcactggcacacctgcagaaacactaaacatttcatatacatatat

SEQ ID NO: 108 - DNA, 469 bp, Homo sapiens
gttttcatgagtatcccgacttccttctaagaacttccacctgagaactgaccacagcgtcacgattccacatgggtgtgtttcctttcccctttcccatttcagtggtttccaatttctttttcttttggcactataaacctttcgcaaaggaaatattagacagaactcctacatgtcaagcaaattaaaatagtggtgaaattagagtggaggacataatcaccctatcatataggctatttgtccatatcatatttgtccctacaaaggcttctaaggcaggggtccccaacctctgggccgcagaccggtaccagtggcctgttaggaactgagccacacagcaggaggtgagcgggagacaagcgggcactactacctgagctctacctcctgtgagatcagcagcagcattagattctcatagaagcgtgaatcctattgtgaactgcacatatgagggatc

SEQ ID NO: 109 - DNA, 312 bp, Homo sapiens
tcactctttccttgtttattaaatatcaacttttcctgcctaatgggctgaggttcattttcccattcctcaaggtaagggtagactacctaggaacttattgcatctttaggccagctggcttagtgctacccatctgaacccccagattactacccaagtcttccttttgccccttcctgccctaacagcaagtaccaggccagtcccttccccagcaaatgccaggggcttcatgtgaagaggaactggccacaaggatgaggggaggaggagaaactgtttctgcaggaaggacagcagtgcctccag

SEQ ID NO: 110 - DNA, 289 bp, Homo sapiens
cctgctcctgctgtgtgtcgggcttattattgtgaccatcctgctgctccagagtgcctttccaggtttcctttagcttccctgcttcctgggaatcaggagcctggacactgccatctctagagcagagtggaggcctggactccctttgctcactccattcgggtccacagcgtgaggttgcctctgacaagatgaatgggcactgcctgcccttctagtgaaaaggcttggctatggccctgtgtgactccaggtcccaggaaccttgccttcgtcatctgtggat

SEQ ID NO: 111 - DNA, 314 bp, Homo sapiens
taagggcttattaaactagttttactttagcaccattcttagtggagcaggattcttgatcatggggtggaattttgtgtatctgggcttcatgggatgcataaaattttccagttggtaagtagcaggtgccgagggtctggatcagaaaaaaaaggcaggcagccgggttcccactcgccttccatgagctctccgcagctcaccggccatcacttgaagcaaacactcctgagggagctggttgaggaaggcgtgcatgagctgcatgccgcggtcccccattgcacactccatgatagtgtctggcgaca

SEQ ID NO: 112 - DNA, 529 bp, Homo sapiens
caggcgtcccctctgcaagcgttagacttctttgggaatgggccaccagttaactacaagacaggcaatctatacctgcgggggcccctgaagaagtccaatgcaccgcttgtcaatgtgaccctctactatgaagcactgtgcggtggctgcctagccttcctgaccattgtctgcatggaagagtttgaggacatggagagaagtctgccactatgcctgcagctctacgccccagggctgtcgccagacactatcatggagtgtgcaatgggggaccgcggcatgcagctcatgcacgccttcctcaaccagctccctcaggagtgtttgcttcaagtgatggccggtgagctgcggagagcttcatggaaggcgagtgggaacccggctgcctgccttttttttctgatccagaacctcgggacctgctacttaccaactggaaaattttatgcatcccatgaagcccagatcacaaaattcacccatgatcaagatccttgtcactaagatggtgccaagtaaa

SEQ ID NO: 113 - DNA, 377 bp, Homo sapiens
tttcttatgcataagaagtacagtttattgggagtgccagataggtaaacaattaattacaatattgtgtacgttcaataacagaggaacatgctgcctgagtcttcctgaggacagaaggctgcaaaatgagagcacaacatctgtgacatcaaatgcactgtgatttggcagttaaacaaaatgtaacctgggctagtagaaaagtcatgaattctgaagtcgaaggaatttgtcttttagatcttgttctgctatttctttcttgtccttggctcaatgcataaatttgttgggccttaagtttcttcctcccagtggcacctggaaccccagtgagatggaaccgttcactcccctggaaagggggctgaagc

SEQ ID NO: 114 - DNA, 397 bp, Homo sapiens
gcctcggtaatgccgggcgaccctccacctaccaagctctatcatttcaggtcaacttcagactgctgtgctggcagtgagaatttcaagccaatggttcttaggtttctgggatgtgtgggagtgggacccactaagcgagaccacttggctccctggcttcagcccctttccaggggagtgaacggttccatctcactggggttccaggtgccactgggaggaagaaacttaaggcccaacaaatttatgcattgagccaaggacaagaaagaaatagcagaacaagatctaaaagacaaattccttcgacttcagaattcatgacttttctactagcccaggttacattttgtttaactgccaaatcacagtgcatttgatgtcacagatgttg

SEQ ID NO: 115 - DNA, 450 bp, Homo sapiens
tttttttttggaaaggtaggaaagctttaataaaaactgaataaaaggtataaaaataaataaattctagcttatcttctcttgaggtcttctgtacttctttgcagatgtcacaaggtttggggatatagaacatgtgatgatgacagagctgggtccatatgaacttttcaaatagagggaatatgaagctaatgagatgtgcagaatcctggaaggacttgggtattaaaggccttgggtatctgaagacatgggtgtgaggaaggcctgcacagagagcttccaatcagcattttagtgcctcatcattaatttggacttataagcaaggatcaccagacacctgagaacatccttaaaacaaaacggaatcacaaagaatccagagaatcaagaggtaatccacagaatggagggaaacatcaaaatacctcttattaatatact

SEQ ID NO: 116 - DNA, 428 bp, Homo sapiens
cagcctcctctggatcttttctggttccttcgctcctagaactgtgtctgatggcttatgaaggcaaatgacaaatgtttgccatatgcacatgtatgtcatgaatgaatacatccaggttctgacttcctcggggcaacctctactttctcaatgcccctcttaagatgggggtgggggagggtcctcctagaactgaacatgatattccaggtgtgatctaatacatttaaatgtccaaaagctctttcttccctaaacgttcctttttaaaagctactgttctcaattaatgggtgtctcatcttcttaagtatattaataagaggtattttgatgtttccctccattctgtggattacctcttgattctctggattctttgtgattccgttttgttttaaggatgttctcaggtgtctggtgat

SEQ ID NO: 117 - DNA, 462 bp, Homo sapiens
tttttttgacatttcatcagctttaatgctgtctcctcaagtaatatggacatgatattaggagagtttcccagaatttatgtgaccattcccaactcagcttctcccctgcttttaagacaagctatagttaggttttcctatcaccaccacagtcctgactttggtaaatttttctctctcacacttagagattccatctcagaccttggcccttcatctttagcaggtgatactgtagtcaccggaccaacccccagccttgatctctctgttggaatcacagtgacgttttcatcacaggcaggcgatctggaagttaagtagttgagaagagagatgatcccagcaaacatgtaaaagatgtcactgcaccagttaagataataggtccataggagatctgactccatggcatccctagtatgccagtgaacctgtgccacaaacaggttgaggcag

SEQ ID NO: 118 - DNA, 559 bp, Homo sapiens
attcggcacaggtcgaagaccgagcgaagggaacatttaaccttgactttccacagtcctgaggttcccaaaatacaggggaacggaaataccaaaggattatctccaatattccagggccttctttctcatctctgtctttaccatacttactggccttggtggctcttcagctcttggatccttaatcgaggaagcatgaccaccaacttggatctgaaggtatccatgctcagcttcatctcagctacctgcttgctcctctgcctcaacctgtttgtggcacaggttcactggcatactagggatgccatggagtcagatctcctatggacctattatcttaactggtgcagtgacatcttttacatgtttgctgggatcatctctcttctcaactacttaacttccagatcgcctgcctgtgatgaaaacgtcactgtgattccaacagagagatcaaggctgggggttggtccggtgactacagtatcacctgctaaagatgaagggccaaggtctgagatggaatctctaagtgtgagagagaaaaatttac

SEQ ID NO: 119 - DNA, 342 bp, Homo sapiens
ttttaacagtgcaaggtaaaatttaatctgtattgctaataagacattttacagcatagaaacatgatcgaagtgatcatttactttatcatgtcataaaacacaagtgcttattgtagaacagtgatataatttaaatggcaaggaaatttgtgattcataacaaagaatcctgtatgtatttgttgccatgattaactttccaggtcaaatatcagtagtaaaataagttgtggtacaaaagaaagccaagaagttacctgtactgaaggaacatttgatttcatttaagaaactgaaggctggattctagttaaccttttgttatgtacaaaaatgttc

SEQ ID NO: 120 - DNA, 486 bp, Homo sapiens
gaagccttatggattggattcgactgaccaaaagtggaaaggatctaacgggattaaaaggcaggttaattgaagtaactgaagaagaacttaagaaacacaacaaaaaagatgattgttggatatgcataagaggtttcgtttataatgtcagcccttatatggagtatcatcctggtggagaagatgaactaatgagagcagcaggatcagatggtactgaactttttgatcaggttcatcgttgggtcaattatgaatccatgctgaaagaatgcctggttggcagaatggccattaaacctgctgttctgaaagactatcgtgaggaggaaaagaaagtcttaaatggcatgcttcccaagagccaagtgacagatacacttgccaaagaaggtcctagttatccaagctatgattggttccaaacagactctttagtcaccattgccatatatactaaacagaaggatatcaatttagact

SEQ ID NO: 121 - DNA, 179 bp, Homo sapiens
tttttgtgataatgtcatttaaggttttattgtaaaagtctattttttctaattaaaacatttttcccactataaaatatttaaacagtgcagatatatgtatatataatacagaattcattagaaatatccactctgttctatatccttctgtttacatatatttttatattaaatac

SEQ ID NO: 122 - DNA, 320 bp, Homo sapiens
  misc_feature (3)..(3): a,t,g,c
gtntctcccnnnnccnccncnntntctctngnannccccccccttncccccccntnncccnnntcccccaaatcaagggagctacatggtgctcactgcattggccgggggtttgacggaggcgagcagggtggcgggctgggtcccggcaaaggagtctggttgaaaacgtatagctgccagggtttcccncttaattcttgttattggtgtaaaaacagaggggttntgcgagaggtggtgggtggcnaaggagggagccaagagaccaagttccggccaaagggtcagcggcatgcagntattctggttacacagtt

SEQ ID NO: 123 - DNA, 271 bp, Homo sapiens
tgctctccctcaaaggaagtgtttattttttggagctcaaagaccccagaaaaaagcaaccccccccgcaaaaaaaacccataacaacaggcagcacatgccccccacaccgcaccccatgcccaatctcagggtgggagacaagaggggggactcattgtccggagagaggctctggagggagacagccctgtgtcaacccgactgtgtccccacacccaggacttccccatggcccctccagacctgctccagaggcccttctccggct

SEQ ID NO: 124 - DNA, 476 bp, Homo sapiens
tcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggaagtcctgggtgtggggacacagtcgggttgacccagggctgtctccctccagagcctccctccggacaatgagtcccccctcttgtctcccaccctgagattgggcatggggtgcggtgtg

SEQ ID NO: 125 - DNA, 476 bp, Homo sapiens
gaggggagatgctgaggtcagggcacttatgtgatcaagtgatggagacagagtaaagagaatttatggatatatatgcatatcttgctaataagtttggtttatcatcacatcaagataaattgcctttagcatagaacttgagataagtagacgttcactagcaagtgctaacatttggatcagggcaaaggcgtttccaggagtgtttttataccagacccctgcttagtccatggggctgaggcatgtcacataactcagagcaagagccttctatttgtgtaagacccaaccctgggaaggatgcccgagacaatagctatgcctctgccccagcctgatgccatgtgatctagagcctgggcagcaacaaccctgtggtcaaagacatggaagaaacactctgcaaatcccacagtctgttcgacggcttggaaaggaagcaagtggcattgggacaattgctctacatg

SEQ ID NO: 126 - DNA, 404 bp, Homo sapiens
gaagccctctccaggcgggcccgccgactggccgtgaggatgcgcgccctggagagctcccagaggccccgcgggtcgccggaccgcgctcccaaaacaacatctacagcgcctgcccgcggcgcgctcgtggagcggacgctgcaggcacaggggaggcccccgttcccggccccggagcgccgttgccccccgccccgctgcaggtgtctgaatctccctggctccatgccccatctctgaagaccagctgtgaatacgtgagcctctaccaccagcctgccgccatgatggaggacagtgattcagatgactacatcaatgttcctgcctgacaactccccagctatcccccaaccccaggctcggactgtggtgccaaggagtctcatctatctgctgat

SEQ ID NO: 127 - DNA, 452 bp, Homo sapiens
taattttaagtttgaatgtatattttgaaataaaaatggaaaataacaaatttgtacaaaattgtcacatagaaacacactttaagataccattacatgctgtgtgtatttacaaaagttacaggtctagtaagaaaaaagaaactgtcattgacaaatgcgagctcattttttgagcaacaagaaatctcccgtgaaatgtcatactgacagccagtgagacttggtgcagtgacggctccgcacctgctgaggcctggagcctgcctcccgggctgagtgctcagctctggcctccgcctggatgccgtctagagtcctatgtgctcataggtctttttctttcagtaaacagtataaaacattactattttcctttgattgtaaacttcaacagaaaataacatacttaagaattttgctacaaagagtactatgatttttattgcctc

SEQ ID NO: 128 - DNA, 201 bp, Homo sapiens
gagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagag

SEQ ID NO: 129 - DNA, 417 bp, Homo sapiens
aatatacataattttattacaaaattttttttaaaaaaacgaaatgcaacatcctaaaaaacccaaaatttactattgatactaattcctacaagtttgctgtgctaccatacacaagaaattaaaaaaaccattaaatatttaggaacattcaacatcagaagctgtaaaatctaactgtatgagtagcccatcaaaaagctacaacctgcattttttaaaagtattttctctacagagaatcttatcagctatacaaaaatctgtacagtttttatactgaagctagtattgagctgcacttgaattcacattcttagcaaaataattgcctgagcacacacacacattccacacgcatcattaaaggatagccatttattcttcatcttcatcctcttcctcctcatcttcatc

SEQ ID NO: 130 - DNA, 468 bp, Homo sapiens
aacgaactgaatgcaacatttttgaaaggagaggtttctgtggaactggaaaggggctctggttgtttccagctcaccaccaaaactggctggccacaaagaacccaggcaggatgtactctgctcagacacaccctcagagggtgggcaggagagagtggacccacaactcctgttagcccagaggaggggttacagctcttgctgcttaacctgagaaatttccagaaatttcctacctgcttctactgcagaataatcatctggcgactgtttctagtgagcatgtcccatgcaacaggctttcctgagcccccgaaagagcatacaagtcaaaggaaaaaggacatgaggatgatttatttggcagtcagatcttaagagggcagcagaactagcaaatggccaaccctgagcccaaatgtgggcagtaggtttgtgtgtgctgaatgctggctatgctatgag

SEQ ID NO: 131 - DNA, 228 bp, Homo sapiens
  misc_feature (169)..(169): a,t,g,c
ggaaacacacttgtaccctgctcccagcatcacaaggcacttgtctacaagtgtgtcccaacacagtcctgggccactttccccaccctgggagcacataaagaagcttgccaaggggggcgtccttgctccccagttgtcctgtttctgtaacttatgatgtcttttncctgagatgggggctcagagggggaaggcctgtnctgcattgcttcccnatggcccacg

SEQ ID NO: 132 - DNA, 323 bp, Homo sapiens
  misc_feature (79)..(79): a,t,g,c
tcatgtctgcctggctgagtcacttcagggatcccgtgctggcaccagagagctgtaagagccacctgcaatgggcagngtgtgtgcctgggacctttcacttccctgcttgtcttctggccgcgtttctgagaggcacgccccccacacacatccatgcacacacgctgtaattggccaacttcctccttctgttcttggaatagcaaccgagacaaaaagcagttagcacgagtggcttgggcttgccaacactacaatttcaggacaaaacccttgtaatttttccaaatggtnggggtttttaaattaggttgggattt

SEQ ID NO: 133 - DNA, 232 bp, Homo sapiens
  misc_feature (1)..(1): a,t,g,c
naaccgttaaacatttttattgtgcaggcttgtaacctttacaggacatgcatgataaaaaattgtaaaatatatcagtaagcatataaaattncagcaatcttttggaccagtaattgatttgcatgagggactaagcatctatgtagtancagacatgtcttggaatacacagtttacagattgantcacaaagccatttcctaagntgattttcttcatataaggctaa

SEQ ID NO: 134 - DNA, 381 bp, Homo sapiens
  misc_feature (1)..(1): a,t,g,c
nattcggcactaggccgngngttggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccatctgttatgctgtgacacatggcccctccccctgccagggagctttgggacctaatccaaggcatccctttgcccagaaagaagatgggggaggaggcagtaattaaaaagattgaagtattttgctgggaataagtttcaaattcttcttgaacttcaaacttgaggggattttcancctgttaaacctgagtccgtaccagnaagctngcctggggg

SEQ ID NO: 135 - DNA, 468 bp, Homo sapiens
  misc_feature (127)..(127): a,t,g,c
ctttgtagtctatacatttattgagtaaaaacaaaatcagtgtcagacacgttatatttgattgggttcaaatttggctgatgtccaaatgcagcagagaagaacggcggggcgaagggacacagtnttgctgacaaggtgacactgaacacaacagttttcctttaattgtaaaagcgggcatcgacagctggtgtgagtcaattaaccaaggcagggaggggactcaatgttttacaagcagagggaaaaccaaagtaggcagagaactttcaagagaggagagggccagaacactgaggaggaaaaagctgcagcagaggccttgctgggagaacagtgggtcagntcttctggcttcacttngggggaacacagttgacccctttcattaaccctcagggtgggtgttgacttcgcttcccggggcatgctttcttcacagtaggttttgatccctnccaccaa

SEQ ID NO: 136 - DNA, 387 bp, Homo sapiens
cagaagttgcctatgtgtgacaaatgtggcactgggattgttggtgtgtttgtgaagctgcgggaccgtcaccgccaccctgagtgttatgtgtgcactgactgtggcaccaacctgaaacagaagggccatttctttgtggaggatcaaatctactgtgagaagcatgcccgggagcgagtcacaccacctgagggttatgaagtggtcactgtgttccccaagtgagccagcagatctgaccactgttctccagcaggcctctgctggcagctttttctctcagtgttctgggccctctcctcttcttgaaagttctctggcctactttggtttttccctctggcttgtaaaaacattgagtccccttccctgccttgggttaat

SEQ ID NO: 137 - DNA, 290 bp, Homo sapiens
  misc_feature (20)..(20): a,t,g,c
gagtttaatgattacatggngctgagtcaggaggtaggaggagattcttaaatctctgaagagttctgggctggggttctgggaggcaaggggctggaaaatttgggccactgattggtcagggtaagggagattgaatcattaggatatggaaattgcattctttgatgatttagcttctggtagggnccttcagaccaggctgacatcagtagtttcatcagtatgcagggncaaccaatcatggccaagtccnctttnagggantttgtncccgtaggatttatccg

SEQ ID NO: 138 - DNA, 417 bp, Homo sapiens
  misc_feature (298)..(298): a,t,g,c
gctgatcgaacagcctcacttgtgttgctgtcagtgccagtagggaggcaggaatgcagcagagaggactcgccatcgtggccttggctgtctgtgcggccctacatgcctcagaagccatacttcccattgcctccagctgttgcacggaggtttcacatcatatttccagaaggctcctgggaaagagtgaatatgtgtcgcatccagagagctgatggggattgtgacttgggctgctgtcatccttcatgttcaagcgcaggaaggaatctgtgttcagcccgcacaaccatantgtttaaggcagtgggatggaaagtggcaagcttgcccaaggaaaangggttaaagggaanttttttgccacagggaaggaaacaccntgggcaagagggnaccatttaccagggggca

SEQ ID NO: 139 - DNA, 442 bp, Homo sapiens
  misc_feature (334)..(334): a,t,g,c
acacttatcccactttttattttttacaaaaacgtgctcatttaatagaaatgattgattatataaaattgtttacacatgtataaaaactaggaacacgtatgtaggtaagcaatcaaaatcatgctttctgttagaaaagttaacacttctgtgaatagactggatgacttccttttagtttggatgtctgggtcggactcacgagacgctggggttggggtgtgggtgcttgacatcagcctctggcaaagcactaataaaagggaaccccaaagctgactgtgtacacaaatggggctttccataaggttcattacatttccttttccangtcagggnaaacttcaacagtgggtngctactgtggggtctgtcccttgnaggtttctggaggccgtgccaantgttaatatccccgcatccatcgtctccgnagtct

SEQ ID NO: 140 - DNA, 380 bp, Homo sapiens
  misc_feature (60)..(60): a,t,g,c
caaccatcttggcttcttcaaacaaagtgaaaagtggggacgacatcagtgtgctctgcntgtcctgggggagcccgatgtggaggtggagttcacctggatcttcccagggcagaaggatgaaaggcctgtgacgatccaagacacttggaggttgatccacagaggactgggacacaccacgagaatctcccagagtgtcattacagtggaagacttcgagacgattgatgcaggatattacatttgcactgcttcagaatcttcaaggacagaccacagtagcttaccactgttgagttttcctgattgggaaaagggaatgttantgaanttatgggaaagcccntttgtgttacacantcagntttggggtttcc

SEQ ID NO: 141 - DNA, 500 bp, Homo sapiens
ggagtgaaaaagacgctgtatttgatttacaatgaacaagatttacaaaaaggggtggggtggtcttggaactgctcccagtccccccggactgggtggggctctagggcagcctgtctgacagaccaggaccccaggatgtctgggccccgacgtaggacttgacctacgtctcacttgacctttgacgtggggcccagcagccgtgagtccacccagagtgccggcacccttggtgaggccggtgaggtcaggaaggcatcgtaccgctttttctcctcctcccatctcgtggtggacagacagacataggatctgggaacttgccctggggcgcacaggctcagtatcccccaggggcccaacctagggcatggaggcggctgctggtgcgtgggcggaggcggaggcagtctgcccccagcgtggcagcgtaaggcacattttcaaatcactcgagactcgacagtgaacacccgatgctggttctgcggccggag

SEQ ID NO: 142 - DNA, 256 bp, Homo sapiens
  misc_feature (53)..(53): a,t,g,c
gtgctgtgaaaaaaaaaaatgctgaacattgcatataacttatattgtaaganatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagaga

SEQ ID NO: 143 - DNA, 434 bp, Homo sapiens
  misc_feature (10)..(10): a,t,g,c
tagcttaagntatttattcaatgctctcacatttggcctctgtgacccagaaaagcggtagagcagggtaggagcacatgatgcatccttcagccactgcaacctccagagggaccaggncttcttcaggaaatcttcgttcctggnggatgactgatcagccataactgcaagacacagacatttagcacctccgnttgcagactaaacacccaccctgactgccctcatcccatgaggntccagggngcctttgccccaagaagcctgggtttccatactacatccctcaccatggggagaagtcctgcgttctccatttctccatcgttgggcttctcctggggagtcatgccaatcattgggtctgggtccccgttcaaagttttccacagggccccacanagttttcccagctggngcatttgttgaca

SEQ ID NO: 144 - DNA, 542 bp, Homo sapiens
  misc_feature (23)..(23): a,t,g,c
gcaaagattcacaatatttattnattctcctccaacattagcataattaaagccaaggaggaggaggggggtgaggtgaaagatgagctggaggaccgcaataggggtaggtcccctgtggaaaaagggtcagaggccaaaggatgggagggggtcaggctgganctgaggagcaggtgggggcacttctccctctaacactctcccctgttgaagctctttgtgacgggcgagctcaggccctgatgggtgacttcgcaggcgtagactttgtgtttctcgtagtctgctttgctcagcgtcagggtgctgctgaggctntagggtgctgtccttgctgtcctgctctgtgacactctcctggggganttacccnatttgggagggcgttatccaccttccactgtactttggcctctctggggatagaagttttttcagcaggcacacaacagaggcanttccagatttncaactgctcatcagatggccgggaagntgaaggncagtnggtgcagccacattncttttgatccnccact

SEQ ID NO: 145 - DNA, 514 bp, Homo sapiens
  misc_feature (390)..(390): a,t,g,c
cagcctcccgcgcctcgctcagctccaacatggcaaaaatctccagccctacagagactgagcggtgcatcgagtccctgattgctgtcttccagaagtatgctggaaaggatggttataactacactctctccaagacagagttcctaagcttcatgaatacagaactagctgccttcacaaagaaccagaaggaccctggtgtccttgaccgcatgatgaagaaactggacaccaacagtgatggtcagctagatttctcagaatttcttaatctgattggtggcctagctatggcttgccatggactccttcctcaaggctgtcccttcccagaagcgggacctggagggaccccttgggccctgggcctttcaaacccaccccctnttcctttccagcctttctgttcatcatnttccacagcccacccttncctggaggcacattaaccacctnatggtagggtnccaactggtcattagttattaaaggnaatgtnaattttttttaa

SEQ ID NO: 146 - DNA, 514 bp, Homo sapiens
  misc_feature (383)..(383): a,t,g,c
ttttttttttttttttttttagtataaacatgattaattagcttttaatactactctttgatgtattactcatattaccaaggaataactggcgggcacagggtcaggtgctgaagggacattgtgagaagtgacctaaaaggcaagaggtgagccctctgtcacgctggcataagggccgcttgagggctctttggtcaagcagtaacgccagtgtctgggaaggcacctgttactcagcagaccatgaaagggcgtctccctttccttggaggagtcagggaacactctgctccaccagcttcttgtgggaggctggatattatccaggcctgcccgcagtcatccggaggcctaacccctccctgtggtgcttcagtggncacactccttgtccactttcatgctcctcttggcctcctggttcccttttgaagctttgagtacatagccagagaagaaatagcgaaagtccttaagtcttgatctttcttttagggccgagaaaaaatgc

SEQ ID NO: 147 - DNA, 192 bp, Homo sapiens
  misc_feature (192)..(192): a,t,g,c
catgtgggaagtgatgagttatggagaacggccttactgggacatgagtgagcaggaggtactaaatgcaatagagcaggagttccggctgcccccgcctccaggctgtcctcctggattacatctacttatgttggacacttggcagaaggaccgtgcccggcggcctcattttgaccagctggtgggctn

SEQ ID NO: 148 - DNA, 370 bp, Homo sapiens
  misc_feature (138)..(138): a,t,g,c
acttgtttgaggtgatttaggattaccaactctgggaaaaaatattcacagccacattataaaaaacattatcggcaggattcatctcctgctcctttagaataagaacttttcttttgtttgtggaactttggaatntacaagcaagacgttcattcatgctttaaaatgctgtttaacaattgcttagcactttaccagggagaatgtttctcctgagtgtttaccaccctattctgtaatacagttgagggnccacatcaangctatctattccttccngggcccagaggaacacacacatttaatctgaggtgcattgaggtaccacaggctgagtgggaggaaagggattaggggagggcaatta

SEQ ID NO: 149 - DNA, 351 bp, Homo sapiens
  misc_feature (253)..(253): a,t,g,c
ggcatatgcatagtgaaaggagtgagggctgcaagttcatggatgcattattttctcgttttcgtttgtagggaaatacatgctctgtcattgacttgcaaggattttctttttattcttaattgcctccctaatcctttctccactcagctgtggtactcaatgcactcagattaaatgtgtgtgttcttctgggccaggaaggaatagatagcattgatgtggaccctcaactgtattacagaatagggtngtaaacactcaggagaaacattctccctgggtaaagtgctaagcaattgttaaacagcattttaaagcatgaatgaacgtcttgcttgtacattccaa

SEQ ID NO: 150 - DNA, 414 bp, Homo sapiens
tgtctgaaagtatgctttttattgcagccataaaccttaatacagatctttttgttattatcaatattatttaatattagtggcagccacatgagcgtactaccatatttctatattttttcaaaatgacattggagctgtcatcaaagtacagaacagagatggcatttaatttggttggagcacaacaaggctttggtacgtggtcaggaaacatcagatgaaccagagtctgaactatagcgtggttggtggcattcatatgggcgttaagtggaaaagaacattctccatcacaataaaatgcagcgtatccttctggtgctataatccagtcctgccatcccagatcccggaagctcacatagagttcgtgcttcttacaggcttgttttgctcacttgtgtataatct

SEQ ID NO: 151 - DNA, 439 bp, Homo sapiens
tgtaactccagttttactttaagaggataatacagatttttgtagctggggaaggtgagtgggaaggtagaggttgtggaaaggctgggaaacatcagaatatatttatgacacaagacatttgaccaacccaagcaatgctttgtgcctgtgcctccagacacacacacacacacacacacgcacacacacacacacaccatgtaaggcaccactggattatagcatggaggggacaaatgccccctgccctaaacctggccacagccagcccatccccctgtgggccctctgtccaagtcctgtggggagcgtggctttgctactcaatggcaactggatttcaagagtttcaggaagggtgggggagcaagatatcaaaggctcaagctcactccccttcgtccagacagacttttcattttttgtttgatgaaga

SEQ ID NO: 152 - DNA, 389 bp, Homo sapiens
  misc_feature (3)..(3): a,t,g,c
aanaaaaaaatctgggaggggcatttttataggacccataaccattgacagttaatatcagccacaataagggaccttcaggagccgctttctagacccagggtctctgggaagcctcatcccacaccctctcctgctgtccagggccatctgggagctggtctgagtgtccactgagtccgtttatttggcggtctgtctcactgggtttgcacgacagtttggacatctctgtgtggctccctgtggctgaaggcttgtcggattttccgtaagaggctcccccagggctgtctatgggtccgtgtttgatatttgggtggatctttggggaacgcgagtccagggagagggtccattcgtggggaaaaccacccagcattgtgtca

SEQ ID NO: 153 - DNA, 474 bp, Homo sapiens
  misc_feature (71)..(71): a,t,g,c
ggctgcggctggagattcgcgcgctcggacgactgacccgccactaccggaaacacacggggcagcgcttnccgctgccagctctgcccacgtgctttttcgcgctctgaccacctggccttgcacatgaagcgccacctttgagccctgccctggcacttggactctcctagtgactggggatgggacaagaagcctgtttggtggtctcttcacacggacgcgcgtgacacaatgctgggtggttttcccacgaatggaccctctcctgggactcgcgttcccaaagatccacccaaatatcaaacacggacccatnagacagccctgggggagcctcttacggaaaatccgacaagcttttagccacagggagccacacagagatgttccaaattttcgttgcaaacccagtgagacagaccgncaattaaacgggttnattggacatttagnccagtttccagatggnct

SEQ ID NO: 154 - DNA, 423 bp, Homo sapiens
  misc_feature (14)..(14): a,t,g,c
ggcacgagcaaganaggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccagggacttagaatagtgcctaaagtgctgcagccaaagacagagcggaactatgaaaagtgggcttggagatggcaggagagcttgtcattgagcctgggcaatttnagcaaactgatgtctgaggatgattcgaggtgggtcttacctcatctactgnaaaattctggtaaggaatgggaggg

SEQ ID NO: 155 - DNA, 477 bp, Homo sapiens
ttttttttcattgccataattttttattatgtatcaaattgtcttcaatataagttacaacttgattaaagttgatagacatttgtatctatttaaagacaaaaaaattcttttatgtacaatatcttgtctagagtctagcaaatatagtacctttcattgcaggatttctgcttaatataacaagcaaaaacaaacaactgaaaaaatataaaccaaagcaaaccaaaccccccgctcaactacaaatgtcaatattgaatgaagcattaaaagacaaacataaagtaacttcagcttttatctagcaatgcagaatgaatactaaaattagtggcaaaaaaacaaacaacaaacaacaaacaaaacaaaacaaacaaacaaaaaatcccaccaatcttcatgggtaaactttcctgctcagggatgtaagctgactctagaccatctcgcggttcctgcggatagcacagcaca

SEQ ID NO: 156 - DNA, 477 bp, Homo sapiens
  misc_feature (277)..(277): a,t,g,c
gtagcgggtgggccctggcccagcgtcccctgccagcatcctcgtgggctcacagaccccggcctcggagcccgtctggcacccagagtgaccacaagtccagcagggaggcggcgccgccatcgccgtgtccgtgttttctttttcagccccggagagggtcctgacctgggggcttctccaagcctcactgcgccagctccccgcccgctctcttttctcccaagcaaaaccaaatgcgccccttcacctcgcgtgcccgtgcgaggccgggggnttctttcagagcccgcgggtcctctcatacatggcttctgtttctgccgagagatctgttttccaattatgaagccggtcggtttggtcagactcccgacaaccacgtnccaggtaaccggtgggaaaagtgggcagttccgagggcgcanatttngggtggttggcagggnccccagaggttncgggttnaactgggga

SEQ ID NO: 157 - DNA, 608 bp, Homo sapiens
  misc_feature (590)..(590): a,t,g,c
atcattttttttttgtttcattttgttttgaacactaagatttattttcaaacagcacacagaccgtctgcggggcagagccaggctaggctggtgtctgggccccacccacagcagctgccaggaaaagaggacccttgcccgggtggcgcggccgaagcttcaggcaagcatggtggctcggcagcccccagccccgccctgcggccaggcacacatgcgggcacaggcaggggcgccagaaactcaactagaggacacagcagcttcaggaacactggtgaattccgccggacttgccgggacgcggctctttggaaaacgacctaatctttgggagaacgcccctctgcctgggggtctcctcttgatttccctttgctcttcaaaagatgaaaaacgaaaacaaaacaaaaaaaagaaccacacatttttcgggaggaggtgttcttcacacgcccggaggctgcctgggccccgccgtcatgggaccctctcagtgaagttcttcgggaagacgccacggcacttctccagctccttgtgctggtccagtcgctcttcttcacgcccatgagccagccttcatnctgctctttaggggtctg

SEQ ID NO: 158 - DNA, 553 bp, Homo sapiens
  misc_feature (391)..(391): a,t,g,c
ttagactgcacactagaatttgattcagaatcaaggtttgtaacaagctgaaagtgactcgtcagttcagttttgcagatagatgagcagtcccttgtttccttggaaactcggacctgagcatatgcagcttggaatgtgcaccttcatgttttcaatcgagcagatacaggatgagtttctcccaagagattcaatatgttgcctttcttttccttgatgtagcttctagtggtgaccaggtggtttcagtccattcaagtctcaagtaaaactccaagtaacgaggtgactgttgcatcttttaaaaatgagtattgtacacatacttcttccaaggcaaatacttagtcaaaactaaacaaatttgtagagggccaaattgtcctgntgtttctcagtttatttatttcataataacagccaccaatacaggtgggtttgaatgttttccaaaaataaacaactactacccaaaataaaacaaacagggttaggatcnggtttcctggtccatacttctcnctgtccttcncactgggggtccctacct

SEQ ID NO: 159 - DNA, 370 bp, Homo sapiens
  misc_feature (277)..(277): a,t,g,c
tttttttttttatggtatatttctttttactcttaaaaaaaaaaaagttcacaatgataacctgcaaacttgcagaaagcgccacgggtttcaagctcctcaccttcggaacatcaccctatccttccccttcccttaaaatcctagatgaaaattcccgagaaagcagaagaggcccccagatgggcggattcccaatcccggacccctgcggcaggtcgaggcattgaagaaataaattaaaggagaggtggctcctgggctggccttgtccagntctgtctcgcagacccagcggtagggcctctgggaggacgtcgtcgttccagcggccgtcgtcgggtgaagtggggcacagtccttcgccttc

SEQ ID NO: 160 - DNA, 413 bp, Homo sapiens
  misc_feature (97)..(97): a,t,g,c
accgcccgcatttcttcatgtacatggttcctcctagactactagggccgccttagcttgctacccttttaggaccctggagctgtgccagggtccnctctgtccccgcgctcctgacaccccctcctcttgcagggccacctcctccccagcccctcctgcagcgtctctgctccggacctcgcctcctcctgctctccctgggcctcagcctncctgctgcttgtggttgtctgtgtgatcggatcccaaaactcccagctgcaggaggagctgcggggcctgagagagacgttcagcaactttcacagcgagcacggaggcccagggtcaagggtttgagcacccaggggaggcatttggggaagaaagatgaattcgttagagttcccagtttggagaaaacagcagaa

SEQ ID NO: 161 - DNA, 442 bp, Homo sapiens
  misc_feature (50)..(50): a,t,g,c
tacttaacagattatatgtgtcctgtgatagcagggttatgttttaaccntctttgcctttcccagtatcttgcaaagagtaaataatttctaaacatatattatcattgttgccatttttcttgataattatgatgacaaggagaagaattttctactttacttccaccatatttaaaaattgaaatacaaactcacaaaaatgggtagtgtcctgattattgtagtcatgcatacatggagtatgtttgaaaggatttgttttaaggcgaaacacagttttttaaattagttaaaaataaaagcatatactatgcactttatgtgcttaaggcatgctatgattataattatagagatattaaaaatgaattttnggccaagtatggtggcttacacctgtaatcccaacactgggagatcgaggtaggcagatcgcttt

SEQ ID NO: 162 - DNA, 466 bp, Homo sapiens
  misc_feature (434)..(434): a,t,g,c
tctctttgtaagtaactttatttttatttacaacagaattggtggctttattcctccatctttagggacacttggcattagcagctagatggaaagtccgcagtgaagtcaaactcattctgccccagccacagctccggaagctcattggctcggtccaaccccagttccaccaccagcgacatcagcacttcctcatccactgggtccgaatcgatgatagcagggctctgggcaccagcagaaggagagagtgattctgcccctcccgcctgggccccaaagtcccagttttgcaggggtcctgcctccccgggttggcctggagtggcaagcagcatcccctgatactgggctattaagtttctgcagctgcatactagccagcaagtgaggggcggggtgcaggttgaaggatgggggtttagtgggangggtggttgtaggagagnctattggagattcc

SEQ ID NO: 163 - DNA, 515 bp, Homo sapiens
  misc_feature (456)..(456): a,t,g,c
cggacttggagtcagagagacctggctgcgaatcccggctccgcacttttctagcgctgtgaccttggttttcaggtggcgaaactgactggtctggttagagcgcgtccaagccaagccgcagtgctgtttggatttttaaatttctccttctgagtgagaaaatagcaatcgagatggaaccatccgcacaacagctccagctggcagcatcacttcccgccaatttatccaacttctgccaaggctctgaaatgccaacaacgtcgaggcctgcacttgatgtcaagggtggcacctcacctgcgaaggaggatgccaaccaagagatgagctccgtggcctactccaaccttgcggtgaaagatcgcaaagcagtggccattctgcactaccctggggtagcctcaaatggaaccaaaggccagtgggggctcccactaagttcctcgggantctccaataggctctcctacaaccaccccttccactaaacccccatccttnaaaccttt

SEQ ID NO: 164 - DNA, 333 bp, Homo sapiens
  misc_feature (177)..(177): a,t,g,c
ttttttttttttctgcaagtggtggaattttaatattgtataaaaaatccaacttgttccacaagtacatatgtcctatgattttatgcatacatccatatacatatatcaaggtaaagtccaatacaaaaaaacagcatttcctatggccagtgttctacagaagtaagactgtgncaaactttatcgtatagtcaaatgagtattgacacactaaggacaggatgaggcagaagcaagttgtgtccacagtatattacaaantaccttgcatagcttattcattctcacctgggtaaattcatcttaggaattctgaagggtttttttcct

SEQ ID NO: 165 - DNA, 404 bp, Homo sapiens
  misc_feature (54)..(54): a,t,g,c
cagagttcactctgagaaacttcaactcagccaaagacatgaaaaaagccgtgnccacatgaaatacatgggaaagggctctatgactgggctggcctgaaacacatgtttgagagaagttttacccaaggagaaggggccaggccttntnccacaagggtgcccagagcagccattgtgttcaccgacggacgggctcaggatgacgtctccgagtgggccagtaaagccaaggccaatggtattcactatgtatgctgttgggggtagggaaaagccattgagggagggaactacaagagatttgcctcttgagcccacaaacaaggcatctctttctattgcccgaaggattttcagcacattggattgaggttaagttgaaaaaacttccaaggaagggc

SEQ ID NO: 166 - DNA, 466 bp, Homo sapiens
ttgacactatatgattccatttatatgaaatatccagaataggtaaagccatagcaacaaagtagattggtgggtttccaggagctgggaggagggggaaataggaacacttgcataatggttatgggtcttgcttttggggtgatgaaaatatttggaactagacagagatgctggttgcacatcatgatatctagttgccacggaattgtttactttaaaatggttaatttcatcttatataaatttcacctccatcaaaatgaaatattttagaaagagaaagttaaaaaaaaaaaacctatgtgttccaccgaacacaacccaacccttgctctcattcccatttcctggttccctgtgtgcccattgtcagtgccatgggtatcaaatccagtgcccattgtcagtgccatgggtatcaaatcagcttaatgatgcagattactgttggggcctgttcctg

SEQ ID NO: 167 - DNA, 453 bp, Homo sapiens
  misc_feature (440)..(440): a,t,g,c
caccatcaaggaggaaggcctggttcttcaagcctatggaaattcctagagacagataaatgtgcaaacctgttcatcttgccaagaaaaatccgcttttctgccacagaaacaaaatattgggaaagagtcttgtggtgaaacacccatcgttctctgctaaaacatttggttgctactcgtgtagactcagcttaagtcatggaattctagaggatgtatctcacaagtaggatcaagaacaagcccaacagtaatctgcatcataagctgatttgataccatggcactgacaatgggcactgatttgataccatggcactgacaatgggcacacaggggaacagggaaattgggaattgagagccaagggtttggggtttgtgttccgtgggaacaccatagggtttttttttttttttaaacttttccccttttcnaaaaatattttca

SEQ ID NO: 168 - DNA, 186 bp, Homo sapiens
  misc_feature (131)..(131): a,t,g,c
aagaggtgtttagtgtaatgagtgttcagatgggtggggcagggtggggttcccccatctgaccctctagggctctggggtgactcattcttccttgggctcagaggtggagctgatatttgttcctccanttgagccctgctccccctggcccttgngtttctcagtgtctccagacactagagc

SEQ ID NO: 169 - DNA, 510 bp, Homo sapiens
  misc_feature (357)..(357): a,t,g,c
ttttttttaaccgttaaacatttttattgtgcaggcttgtaacctttacaggacatgcatgataaaaaattgtaaaatatatcagtaagcatataaaatttcagcaatcttttggaccagtaattgatttgcatgagggactaagcatctatgtagaacagacatgtcttggaatacacagtttacagattgaatcacaaagccatttcctaagtgattttcttcatataaagataaaaatatctttacatttaaaaagtatgcagtatagcacattttcagttatgtacacagtttaaaagtaaaactatatccatgccagtgtctgttttaaatgcaaaaagtcaaagtaggtancaggttggtaattaaagtgtcaggaagctggaagaggcaaaaacaaccagagtncaataagtgtatgaaaaaaaaanatactgaggtttaagaaagncccaagcagaanccattgacagaggaataaaactttggatatccagcanttctgtc

SEQ ID NO: 170 - DNA, 448 bp, Homo sapiens
  misc_feature (432)..(432): a, t, g, c
ccaaactaatccatcaccggggtggtttagtggctcaacattgtgttcccatttcagctgatcagtgggcctccaaggaggggctgtaaaatggaggccattgtgtgagcctatcagagttgctgcaaacctgacccctgctcagtaaagcacttgcaaccgtctgttatgctgtgacacatggcccctccccctgccaggaagctttggacctaatcccaaagcaatctctttgcccagaaagaagatgggggaaggaggcagtaataaaaagattgaagtattttgctggaataagttcaaattcttctgaactcaaactgaggaaatttcacctgtaaaccctgagtcggtacagaaagctgcctggtatatccaaaagctttttaattcctccctggctcatattgtggattccggccttttggggancttttccttaaacctt

SEQ ID NO: 171 - DNA, 377 bp, Homo sapiens
  misc_feature (97)..(97): a,t,g,c
ggggttttaccagttttatttctagactttcatgtttgtctttttgtcttctgctggaaacatgccggttacatgttggtgctgggaagcgccgcgntgcaaccagaaatgcacagacccagccgcccgccgcccagaccctcagacttgcgcgtcacaggacagactccgctgtgccccgtgcacttgccaccagcctttgnctctcgatacacacaacatccaggacttgtgcccttgccccatcacgacagacaaagcgtccctcaaggcccccgcgtggttcagacagacgccgcagccaggatggttcgaggtaagcgtganccgtngttctggaggggtggggcggggtgggcctgtgccgaagatggcct

SEQ ID NO: 172 - DNA, 434 bp, Homo sapiens
agctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctaagcaagctgcccgaaccaaaagaatttgcaccccgctgcgggcccaccgtggttggggcccctgccaatggcagggtcaatcctgtgctcggaggccatctcgggcacaggccacccccgccccacccctccagaacacggctcacgcttacctcaaccatcctgggctgcgg

SEQ ID NO: 173 - DNA, 413 bp, Homo sapiens
  misc_feature (409)..(409): a, t, g, c
attatgccagagaaatgaagtttattgggaagtaaatgaagtctcctccttgcattccagagaatgccaaagaaaaaaggaaaacaggaaatgcagtagtacgttcaggaaacaccttaagtagtaactgggagctcagattacagggagctgaacatctttagaaacaaacaggctcgaccctcaacaggaaggagttgtccagacattggaagttgatgatgtgtgtctttgcttgggtcaggaaagcttttcagcattctagaaataacatagaggcaagatgaggtttcttgatgtcagctgagctccagaaaagtcaggacttgaggatccttcctgtggttgatctaaatggctttgtagatgtcttcaaagaggatacaagcttttcaattacagcaacccntttg

SEQ ID NO: 174 - DNA, 464 bp, Homo sapiens
ctggagagtgaggactgcaagctcccctgcaacccatgcgccaccaccaatgctagtggcaactcctgtggaccctgtggcacctctcaaaagggttgctgtaattgaaaagcttgtatcctctttgaagacatctacaaagccatttagatcaaccacaggaaggatcctcaagtcctcgacttttctggagctcagctgacatcaagaaacctcatcttgcctctatgttatttctagaatgctgaaaagctttcctgacccaagcaaagacacacatcatcaacttccaatgtctggacaactccttcctgttgagggtcgagcctgtttgtttctaaagatgttcagctccctgtaatctgagctccagttactacttaaggtgtttcctgaaccgtactactgcatttcctgttttccttttttctttggcattctctggaatgcaaggaggagacttc

SEQ ID NO: 175 - DNA, 368 bp, Homo sapiens
  misc_feature (11)..(11): a,t,g,c
ctgaaaaaatngaaggcacatttattaaatgactgggagaaattccanagtatgtagaatgggaataataatacataacattgtattttatgttccattttttaaaatgagtccaaggaagttaaaatattcttttaattaagacactcaaagaaatggaaataagaaaaattgatgcaaggactccttcaagttaagatttgtnatacaaatattttcatcttttaacagggcaagctgatgtgttcacatctcagtttcaagctgcctctttcactaggaacatcagtatttttttttaaaagcacattttacaatgctttcccatcacccttggctgtggtttntngntagcacctattagccat

SEQ ID NO: 176 - DNA, 426 bp, Homo sapiens
  misc_feature (378)..(378): a,t,g,c
ctgggaaaagtttctttaataaaaaagttctagtacatatacacgattgtcctcacccttcatctatagcaacgcaacagggaaaataaaaaataaggggcaacctaggcacactcagtataaaaacgcagagatccatccgaatgggaggcattcggggtctggaaaccagaaatgcaggacggccagtgggccagcagctctgggctgcacttttgaagaactttctctaacgttttgaagatagcattaaaaaaaaaaattaagttgcaccaggaggcctaagaaggttccattccagagagaaagtccactcgtaaggtcagtcacagcaggcccaggcccaggacacagggttcgagcattttacggcaggtnaagaggcttctggaaacttggcctctgaggcaaccctggcccaggtga

SEQ ID NO: 177 - DNA, 437 bp, Homo sapiens
  misc_feature (26)..(26): a,t,g,c
gcgacgactaggcggcctgctgcagnngggcggaccaaacgtcaggccacagagcatctccaaggggtctcagttcccccttcagctgaggacttcggagcttgtcaggaagtggccgtagcaacttggcggagaaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagttggactttctctctggaatggaaccttcttaggcctcctggtgcaacttaatttttntttttaatgctatcttcaaaacgttagagaaagttcttcaaaagg

SEQ ID NO: 178 - DNA, 483 bp, Homo sapiens
tttttttttttttttttttttttttttttttcaccatcagtatcatttttattgcaaatcagttaacaaaaaagatgaaaaaaatacatcatctacagttcctcatgtagatcacttttaaagtctttttctgtaaactacactctattttataaaacacagtaaaaatcaacatcttgggacgcatctggtcatgccccctggggatggcaccacgcggcccccgtagagccgggggaggctgccctgaggagtgcaggcggcacggcagcggagttcacccagtgcatgggcggctaaggctccccgagctgagccgagtctgacgtccctcactggcatgacacacaacagactcattcattaagattttttaaacaaaatccacctttaaaaacatatttacagacattttttctgccataggctaatacttggtattggcaaaaatctgctcctcccaaactagggaggggtggccca

SEQ ID NO: 179 - DNA, 268 bp, Homo sapiens
  misc_feature (23)..(23): a,t,g,c
ctgaagtgggagcggcccttgcncaccctcctagtttggnaggagcagatttttgcaataccaagtatagcctatgcagaaaaaatgtctgtaaatatgtttttaaaggtggattttgtttaaaaaatcttaatgaatgagtctgttgtgtgtcatgccagtgagggacgtcagactccggctcagctcggggagccttagccgcccatgcactggggacgctccgctgccgtgccgcctgcactcctcagggcagcctcccccggnt

SEQ ID NO: 180 - DNA, 480 bp, Homo sapiens
  misc_feature (273)..(273): a,t,g,c
cattaaaaggaagcatggagttctaatgctcccataaactatgtattttggcaagacacttcactactccaggtctcactttccccatctgtaaaacagggtttggactaggtgttccctggtattctgtgatctgcctcttgctgccattctttctctcctctgcttctctgtatttttcttctgttatccctgggggtgctcaggttcacttgattgtctgtatttctgtgtggttgtagcaaggactcagcctcatgtaagcacgaatanggggtgtggttcatggcgtgttgacccagcagancactccctcccactaactttgttctgcatgtgtaanagtctccccattttttttaacgcaaccctttcccctttttcctaccccacagtctgttccatgtaagttgccaacagtttcactgaacagtggggtatgtgatggttttggcatgacatcttcagtatgaaggggan

SEQ ID NO: 181 - DNA, 274 bp, Homo sapiens
  misc_feature (229)..(229): a,t,g,c
taacaggaaaaaattatttaatagtataacaaaatgcaaaataaagtacccaagttacaaaacataaattcctttggttcatgatcacaccactatttttaccttccacatagctacagacatcacaccctcaaagtgaagtcaaactgtccccctcatactgaagatgtcatgccaaaaccatcacataccccactgttcagtgaaactgttggcaacttacatgggnacagagntgttgggggtgggganaaagggggangggggggngtgg

SEQ ID NO: 182 - DNA, 503 bp, Homo sapiens
  misc_feature (302)..(302): a,t,g,c
tatgagttcaatatttttatttctttacaatgatttcagaagagattacaaagagattaatatacttaaagaatcagactcttgcaaacagtgacatcattaaaaagagcttattttcattaacatgtgattaacaggaaggagatgattggtgagttttcttcgtaaccaggttcactgtggataggaagggcctgccttccttcccaccatggagatcctaaaatcacaagctccagcctccatcaatgatgacagggttaccagttacataagcagattcatcagaagccaaatacacngcagagcatggctatttcttctggcagttgcgaatcttcccgtcttttggtctcttcaggaaatcattcccgtgcctcttcaggatttcctctggcttggtattcctttcctgtagagatgggcggatcaactggtnccgggcacacacagttggcacctggatgccctggctgggatggaaatctggcagccacaggatt

SEQ ID NO: 183 - DNA, 482 bp, Homo sapiens
  misc_feature (64)..(64): a,t,g,c
atttccctagatccagaaactgactctattgaaggaaaaaaatctgatatcaatctttttaaanggggaggaatgtgggagaaagcatgaaaatggctactgggaaaacttatttgtgttacctttctgaaggaaaatacattttttattccttcaattgttgaacctttcctccaccctcaggagttgtgaacagatgtgtgtacagcacaaccaaggcagccgtgattggcctcacaaaatctgtggctgcaagatttcatccagncagggcatcagggttgcaacttgtgttgttgcccagggaacaggttgaatacgccatctcctacanagaaagaattncaagccagagggnaatcctgaagagggcaccggaatggatttccctggaaggggaccaaaagaccggggaagattcgccaactgccaggaaggaaattagccatggctctggcggggtattttgggctncctgagtg

SEQ ID NO: 184 - DNA, 438 bp, Homo sapiens
  misc_feature (438)..(438): a,t,g,c
ttttagtttggtctgaataaaatccagcataaatgtattattttattctgggtataatggctcggtgtctttgtcccaacagctttcttcttaagggcaaaccacccttaagtgaaacttgccatttgtttttgttttctttttaagtctccagatcttgctaggacacttcttccagaccaacagtcagatagttgtggcacgtgatcctatttgaaatagaggtggtttaaatcagccaagcttggtgatctggaggtctccatttatctttatggtgtcaattgcagataacgtttgaatgcgatggtaaaaatcaaaaagttggtgtccatccacaaacactcggaaacgtgggtgctcacaaagaatttcccacctggaagaaaccaaaataataatcccccatttcaatggacgctactgggggaaacaaan

SEQ ID NO: 185 - DNA, 465 bp, Homo sapiens
cggaaaaagcttttacacttacattctacacaggaatcagaaattttggaggtagagcccaacaatctgtgttctaacaagccctctgcaggtctcaatgcattctgatgtttgtgaaccactgaagatactgcctttctttctctaaaatgaagtattgtttgttttccccagtagccgttcattgaaattgtggattattattttgtttcttccaggtggaaattctttgtgagcacccacgtttccgagtgtttgtggatggacaccaactttttgatttttaccatcgcattcaaaccgttatctgcaattgacaccataaagataaatggagacctccagatcaccaagcttggctgatttaaaccacctctatttcaaataggatcacgtgccacaactatctggactgttggtctggaagaagtgtcctagcaagatctggagacttaaaaagaaaac

SEQ ID NO: 186 - DNA, 468 bp, Homo sapiens
  misc_feature (446)..(446): a,t,g,c
gagggcatattgataaatctttattgacaaaatattgacattgacatacttcttggaagtatatagtgtgttagaattctaacaaattaacacaaaacacaaaaatatttacattctggtatagaagacattaaggaagcatttgtcactctctttagtaagtctatgatcttggaatagaaactcagtgcttgaaaacttgccgccgtgcctttggccacacttaacatcatccccgctaactacagtccttcaggttttgcaatagatagatttaaagtttggaataggcattgcagtgaatggttgaactcggccaatttctccaaccactgaaaggagaagtttgcatcagggttttaagcctcaggatgttaggaaagggaattgtccaagaaatataattaatttaggggttttttttcccagtaccaagtcctgattcntttttgtgggcncccttccccc

SEQ ID NO: 187 - DNA, 401 bp, Homo sapiens
  misc_feature (22)..(22): a,t,g,c
agctgaacgtgcactctgacanggggagctgtcaatcaaacaccaaaccggggagacaagatgattggcaggtattccgtttatcacagtccacttaaaaaatgatgatgatgataaaaaccacgacccaaccaggcacaggacttttttgttttttgcacttcgctgtgtttcccccccatctttaaaaataattagtaataaaaaacaaaaattccatatctagccccatcccacacctgtttcaaatccttgaaatgcatgtagcagttgttgggcgaatggtgtttaaagaccgaaaatgaattgtaattttcttttccttttaaagacaggttctgtgtgctttttattttgattttttttcccaagaaatgtgcagtctgtaaacactttttgat

SEQ ID NO: 188 - DNA, 469 bp, Homo sapiens
  misc_feature (356)..(356): a,t,g,c
ttttttaaaaatgatgaatattttatttttcagacgtccatattttaaatgtaatagttttataaaagaaaaggttggcaacctgttaaggagatcttcatgtgaaaaatacatgtagaagttttaaaaatttgtggatataattgtcattcagaattaagcaggttgattgctgttatctagatgggtctcttcctttatgtttttcagtcacataatcttgatttccatagttatcacatgtacttaaagaagttaatcaatgcctatatggtccaaggtataatttgcacacagtagtttttggtttttatattgtgggatcatatgtatcaaaggtaatatttcataaagancaatggttatagttggctacaaaggggaccaattccacatttcctatacagggaatattttaaggggtagagttatatccggcnatgggtgattggggtttaaatatcccngc

SEQ ID NO: 189 - DNA, 364 bp, Homo sapiens
  misc_feature (16)..(16): a,t,g,c
ggctctttcaatcctnggtgtgattgttgccaacatgacagaatggaagcaccagcgcatcaccaagcaattggagaaatggatgcaaaagaatgtggaggaaaacctctatagcagctccctgggaggaggggtggccacctccccagtgctgattgtgtttcatgggaaatattccacaattaaccccctgtggcacataaggcacctgggctggaatccagatgccagatattcgggagcattttctgcaggaangctaaattactccactggaatggaagacataaacctttgggacttcccctagtgttcacaacgacttaatgggaaagctggtttgttcctgaccctggcagggata

SEQ ID NO: 190 - DNA, 451 bp, Homo sapiens
  misc_feature (6)..(6): a,t,g,c
tttttnttctgcaggtttccttttaatcaaggctctactgaaggtgttttgtggggctaaaagccataaacatgaaatggacatgtaacaccacctggatcccccatagcaggccagaccactctggcgagcactgctggtctgcccaaatctgggtaatcagactgggtattcattggctgcatttcaaagcacagcactgctttcagccaggatgaagtgggagtgaacccagctgctagcagagtgccactccaggctgaagagccaagtaccagccactgccagtgaagactggcccctttaactgaaggaagttgttcanagttccagccaccgggccctggggganggaagagaaantcagggtaattctgctccggggatgggtcagggttccgcaagtttcaatnggccaggcatcctttggaaaagcccgncttctggntga

SEQ ID NO: 191 - DNA, 506 bp, Homo sapiens
  misc_feature (38)..(38): a,t,g,c
ccacgacccccaggaggattgaaggagaccgggagctngccggcgtggacngnnggaatggaggggctggggctcggcgggaggcacccccacagccgccccgggangagcgcgcccagcagctgctgacgggttggagcagcggcacgctagctcctggacaccatcgcagcctgcanggagatgttacggcagctgggccgccgngcccggagccgtngngtnggcgggaacgtctcagccaaacctggagcgcccccccagccggctgtctccgccagagnntntntccaaaggantgctggcgatggaagctgcggacccgtgaccantccccgagcagaataccctgacttctctccctccccagggcccggtgnttggactctgaacaactcccttcagtaaaggggccagtcttcactggcagtggctggtacttggctctcagctggagtggcagctctgctagcagctgggttcactcccactttcatcctggctga

SEQ ID NO: 192 - DNA, 350 bp, Homo sapiens
  misc_feature (254)..(254): a,t,g,c
gttgtaatatgcattttatcgtgtcacttaagtactaagaggtgcttttaaagtttttattaatgctgctcaaacttgaccccgacggtccaggatacaggccaaggtgtcagtgcggacacaatcgcaaggcggctgttaggcttcagggctgggcaggaaacttgccaggtgctactaggaacgtatcagccctttctacaaagcaggtgtctttcccatcataaaatgcaaagcaatcagccgttcccttnctccccagngccctcgtccggagaccgagaggatatttaccctgggctcttgcagttctcttataactgaacccagcggcggttccttctccgagn

SEQ ID NO: 193 - DNA, 508 bp, Homo sapiens
  misc_feature (356)..(356): a,t,g,c
tagtttgtttaaagactttatttgaaaccaatgtcacagcacgttagtcataaaaatcatcagaatcatcggtgttatggtttctggggcgtaaagcagcctcaatttctgagttactgtcatcagactcatcccagaggcattagattttaaattgatcacgggtttctttttgttttcatgccttgacaaaggacttccttcactagatgaaaaactagcttccgaagcaattttgttagctgtctttgtctcgaatgcaccgctgccaatattcaaacctgacaagtgtgacacagaagcatctgtcttttgaaacctgttttcagttttgatttgtattatcctgaagggtnggccttcttcagcaactgataggcttctgtttctgtgattgggatatctgcagccacattttctttcttggctcttgtctccacagccacttgaaacntcttgtncccttgtaggaacactgttgagccatacagcaggtttggcttcctgn

SEQ ID NO: 194 - DNA, 456 bp, Homo sapiens
  misc_feature (398)..(398): a,t,g,c
agtgaccttaccatttcaataagtgaagatgatctgattttagagagcccagaaccacagccaaatccaggtggcaagatggagggagaagatggaatagaggccttaaaattaatccatgctgagcaagaaagagttgccctatccactgaaaaaaattgtattttgcaaaccctaagctctcctgattcagaaaaggaatcctccactaacgcaccaacaagagaacctggacaaacaccagactcagacgtaccgagggcacaggtgggtcagcatgttgccaccttgaaagaacatgataattctgtcaaagaagaggcaacagcattattgagaaaagcccttacagaagagtgtggcgtagtcagctattcacagtagtgatcatcttgcanttgcatctattctgaatgacaatagtggaataaaggaancaacctgctgtatggctca

SEQ ID NO: 195 - DNA, 610 bp, Homo sapiens
  misc_feature (521)..(521): a,t,g,c
tttttttttttttcttgcgttgcttttatttaatcacttccccagcccttccatcgccttttttgaggataagaaacccaaagttcccagagattaacttgcccaagatcacttggcctggatttgagcctgaggtcagactccaaaacccacactcaatttgctacatctcccaggggtggggccaaggcagctgtgagcaaaaggagaagtatcagcttctcaagggcctagggtttgttggaagggcaaggcaagggcaaagggggatacagaacaagggggcaagtaccagtgcctgggatggacccatccattcaggcagggggtgtggggtgtcccctgtgcttagaaaccacctagcatcatagctgcaacagcactttattgggatcttagtctacagttcacatagggaggtgaagccgtgggagaagcaggggtaaaaaaaaaaaggggggggacttcaccccctagggacagctgcttccaaacctaacaaaaccccagggtaagtcccngtgctgggcctcgagcagcaactctagtcaaatcccaaggcaccggtcaaccatgtgggtcaaagggcccctnttgggacntcactcaa

SEQ ID NO: 196 - DNA, 534 bp, Homo sapiens
  misc_feature (319)..(319): a,t,g,c
aggcaatctgattctgaagctaaagagctttcatcctcttgagtgtatgtccccatagtgggccccttgacccacatgctgaccggtgccttgggatttgactagagttgctggctcgaggcccagcacgaggacttaccctggggttttgttaggtttggaagcagctgtccctagggggtgaagtcccccccctttttttttttacccctgcttctcccacggcttcacctccctatgtgaactgtagactaagatcccaataaagtgctgttgcagctatgatgctaggtggtttctaagcacaggggacacccanaccccctgcctgaatggatgggtcaatccaggcactggtacttggccccttgttctgaatcccctttggccttgccttgncttccaacaaacctaaggccttgagaagctgatactctccttttgcnaaagntgccttggnccaacccctgggagatgtagcaaatgagggggggtttggagtttagnctaaggtnaaatcaggccagtaatttg

SEQ ID NO: 197 - DNA, 455 bp, Homo sapiens
  misc_feature (131)..(131): a,t,g,c
ctctgaagtggtgaattttaatattgtataaaaaatccaacttgttccacaagttacatatgtcctatgattttatgcatacatccatatacatatatcaaggtaaagtccagtacaaaaaaacagcattncctatggccagtgttctacagaagtaagactgtgcaaactttatcgtatagtcaaatgagattgcacactaaggcaggatgaggcagaagcaagttgtgtccacagtatattacaaaataccttgcatagcttattcattctcacctggtaaattcatcttagaattctgaaggatttttttcctagataaattttatacaagttagtgtatacttcttgtctttggttctgtggcaaaccaggtttctcagtactgattgttttaccttcacaacattattggatttaaccantagcccgagctttgggggctctgcactgcg

SEQ ID NO: 198 - DNA, 374 bp, Homo sapiens
  misc_feature (141)..(141): a,t,g,c
ctaattttgcagtgcaacacagatatctgtttgaagaagacaatcttttacggtctacacaaaagctttcccattcaacaaaaccttcaggaagccctttggaagaaaaacacgatcaatgcaaatgtgaaaaccttatantgttccagaaccttgcaaacgaagaagtaagaaaattaacacagcgcttagaagaaatgacacagagaatggaagccctggaaaatcgcctgagatacagatgaagattagaaatcgcgacacatttgtagtcattgtanncggattacaatgaacgcagtnaagancccaaagctcaggctattgttaaatcnataatgttgtgaagtaaacaatcagtctgagaacctggt

SEQ ID NO: 199 - DNA, 303 bp, Homo sapiens
  misc_feature (62)..(62): a,t,g,c
ctaaagggaggcaaagttgcactctccttccccagaggcttcaccaagagggcacacccccngggtntctggtgggcaacgggggtgagcatgtccctgccctggctccctccatctgtgaccaggaggcatggctgggtgtatgttcaggtgaggctcagagtngcattgtgtccctgtcccctgcccagggcantgnaggggagcccttnatgctgattagaaggctagaactggggtagaggtgcctggcatgtctcatgccatggggactcaatctagcaactgtgagtcctgggggtc

SEQ ID NO: 200 - DNA, 452 bp, Homo sapiens
  misc_feature (319)..(319): a,t,g,c
tgaagagaaagaaaaatcaaatatttattaaaagtaccataatacagacccatttcaagtaaggacaaacacaccaacatatttcttagtagtttcctcacaatagattattaaagcatagaacaattattcatattcataaagaaatgacttcaaaataggttaaattgttttccatctactctgtttaataaggcaagaacaaatgattcactttagacaaatagtctcatcaaaaaagggctaaaatagtaaagattcatcacctaaagtggtaagctttggatatctgaaatataaacatgttagtactctgatngatgccagataaatgaatttaggcaagaaaacacattgttacaaaaagcctggggttctaaatcagggattactgagacactaacaatttcagatttttgcctcattccagaagcaccnacccagttttcttt

SEQ ID NO: 201 - DNA, 257 bp, Homo sapiens
  misc_feature (45)..(45): a,t,g,c
gtcaatttcctataagaatattattctgaagacctggaggaggangtagcagcagcgctgaaaaatgggaatgacttctaatagtttacaatatcatgaaggaagggctgttgaattgtagcacaaacctgaacctggtcactgactgcatggaacatngtctgacatcggtgcatccgcgaactcgatattcagctggctgggatgctaaatttttcttcatccctctatcttagttacctacatcactggnagac

SEQ ID NO: 202 - DNA, 318 bp, Homo sapiens
  misc_feature (161)..(161): a,t,g,c
tctagaaatggactgtcataaaaaaatgccagatcccacctgtgacctgacagcccccaattcctagagaggtgagagctgtcgtgtggcaggttaggagaggtgccaaccttgggcgatgcggcttcttgggaaataacagctccagcgagaaagaattnggggtgagggagaggatgggggccagaatggggggacncttaagtggtaccaaaattagctgccgtctgctccctgctggtgggtccttggggtggaaagcatggcagggagggggttggggctgaatggagaagaaagtgagatgctgataaaaaa

SEQ ID NO: 203 - DNA, 268 bp, Homo sapiens
tatcagcatctcactttcttctccattcagccccaaccccctccctgccatgctttccaccccaaggacccaccagcagggagcagacggcagctaattttggtaccacttaagggtccccccattctggcccccatcctctccctcaccccaattctttctcgctggagctgttatttcccaagaagccgcatcgcccaaggttggcacctctcctaacctgccacacgacagctctcacctctctaggaattgggggctgtcaggt

SEQ ID NO: 204 - DNA, 403 bp, Homo sapiens
  misc_feature (403)..(403): a,t,g,c
caaatgtataaaccttttatttaaaattagacagactcaaatacaaattaaataagtacaatataaaatagtaagccttcataaaacacatcataaccactttaaatcatttactatttctacagcaaaattaaagagaaatctaaatacagtccaaaatcaaattatttttaaactcttattgtcaacattaaaattattacaaaacattcaaagatatttaattagaagatgaacaaggccaggtgtagtggcccgagagttgtcgatatgctcgcaggaagtgtttctgtgttaaaaagttgagaagatggaaactgaatcctctttgtattcagaaggctgtttcggaaggtcactgttggcaggcttctccctacagggattcagcagtgagggagcn

SEQ ID NO: 205 - DNA, 479 bp, Homo sapiens
  misc_feature (308)..(308): a,t,g,c
caagacttcaaaacccattttcttgacccaaagcttatgaaagaagaacaaatgtcacaggcccagctcttcaccagaagctttgatgatggcctgggctttgaatacgtgatgttctacaatgacattgagaaaaggatggtttgcttatttcaaggaggcccttacctggaaggaccacctggattcattcatggaggtgccattgcaaccatgattgatgctactgttggtatgtgtgcaatgatggctgggggaatcgtcatgactgccaatctcaacatcaattataaaagacctatccctcntttgttctgttgttatgataaatagccaacttgataaaagtttgaaaggaaggaaattttttggtttcctgtaatgttccagaagtgttggatgaagaagaccctatactccagaggcgacaagcttaatttataaagccgaatcccggctaaaaagtctgacnataaagg

SEQ ID NO: 206 - DNA, 450 bp, Homo sapiens
aagtttaagttatttatttaagccatctcctgccacaagcaaagggatgggagaccagtataccagtcatcatttgatacaagtaaagaaggtgagcttaaggtcccatatactttttctggtccatgtgcagagtaacggtccaaaccagtgctgtggtccctggtgcccgggtagtggtccagattctctttattcatcttcgtagccagttggaagtggattcacatgagggttatggaatagagtatggttaccatctccccagggaaacggcttggtcctgatgcggagatgggggtaggcgatgaactcgggtctctcgtgctctccgtggtgcgacttcaggtacacattcagcatgctgactgccaccccggggagcgcgacgaagaaggtgagagtcttccacatggcgagctgagccctcttcgccatgggcccaactcg

SEQ ID NO: 207 - DNA, 349 bp, Homo sapiens
  misc_feature (82)..(82): a,t,g,c
ggtggcagtgtcctcggtttctcggctgctgggtcggtcccgcccacagctggggcggcctatgtcgagtggcgcccatggnaangagggctcagctcgcatgtggaagactctcaccttcttcgtcgcgctccccggggtggcagtcagcatgctgaatgtgtacctgaagtcgcaccacggagagcacgagagaccccgagttcatcgcctacccccatctccgcatcaaggaccaaagccgtttcccttggggaagaatgggtaaccantactctanttccataaccctcatgttgaatccactttccaacttggctaacgaagattgaataaaagagaatcttgg

SEQ ID NO: 208 - DNA, 341 bp, Homo sapiens
  misc_feature (162)..(162): a,t,g,c
tttttgacccaaacaaagtttcatttacagtgtatacagtcaggccttgggggcaggagctgctggggagcagagagggaccctctatcgctggcttcagggggtgccttgggtcccttcagtagctggtgggcgtgactggcaggaagcacatttggcttnggcaggagctgtcaggaaggggaagcgggtttcctgcgattctgccttgttctgccctgggtgaagccgcacaccacacctcacctcccaggggcccaaggcctgttggaaatgtctgttttaaagctccctgggggctgggcctggcttgacggcagcttgcaaaggggaatgccnca

SEQ ID NO: 209 - DNA, 287 bp, Homo sapiens
  misc_feature (190)..(190): a,t,g,c
ctgcccgggcccaaggggtgcacagggcactccatggctccattattaacacaactctagcaattatggaccataagcacttccctccagcccacaagtcacagcctggtgccgaggctctcctcaccagccacccagggagtcacctccctcagcctcccgcctggcccacacggaggctctggctgtnctctttctccacttcatttgctttggctctttctacacntcnctcttgggcatgggctgagggctggagcgagtccctcaagnaatttcaccaggct

SEQ ID NO: 210 - DNA, 488 bp, Homo sapiens
  misc_feature (394)..(394): a,t,g,c
tggaagttaaagtatttattgatgtgtttaaactgtgtacattctccacagatcatattaaggagtttgtaggtgaagtttaatctgtgcatagtgggtagagacatgaatagggtcaaaggggaggaaaaaggaaaaaaacaaaacaaaaacagtcacaggaaaataaaaatacaccacaggttaccagaaccttcagatttaaaataaaaagaaagaaaaagcagaagcagtgagcatcgacatcaactgtacaagcattacaaagactcctgtgacgaaaacacaattgttcaaagctttccacagtgtccacagactcagtttcagggacagcctggactactttcctttcacacaaacaaacctccccgtgttctctcctgggccagcnggccctcccttggtgtggtcttctctgagtgaatgtcacaaggccggtgacaggagggggtggaggtnaggggcaaantngagccnagggtcag

SEQ ID NO: 211 - DNA, 478 bp, Homo sapiens
  misc_feature (7)..(7): a,t,g,c
tgacccntcggccntctactttgtccccntcacctccncccccntccntgtcaccggccttgtgacattcacgtcagagaagaccacaccaaggaggcggccgctgnccaggagagaacacggggaggtttgtttgtgtgaaaggaaagtagtccaggctgncctgaaacgtgagtctgtggacactgtggaaagctttgaacaattgtgtttncgncacaggagnctttgtaatgcttgtacagttgatgtcgatgctcactgcttctgcttttnctttctttttattttaaatctgaaggttctggtaacctgtggtgtatttttantttcctgtgactgnttttgntttgnttttttncnnttttcgncccnnttgaccntaatcaangtctctacccactattcncaggttaacntcanctacaaactccttnatatgtctgtggngaatgtacacaggtttaacacancataa

SEQ ID NO: 212 - DNA, 214 bp, Homo sapiens
  misc_feature (138)..(138): a,t,g,c
ggtttaaatactgcactctattttttaaggactctgtgcatggtatttccggtggcagatgtgtaagattttttatgtaaatcgtgttgttttagcagaggactatcagacctgacagcagctggacatcgaggtccnccacttgggggtggtgacttgncacgcntgaatgctgaagtggaagccaagcccccttcagtcagagctggggcng

SEQ ID NO: 213 - DNA, 391 bp, Homo sapiens
  misc_feature (8)..(8): a,t,g,c
cgtttctncgcgggctcccggcntgctactgctgctgctcttcctcgggccctggcttngncgagccacggcgtcaagtactcgcgggagaagaaccagcccaagccgtccccgaaacgcgagtccggcgaagaactggacaagctctggcgngagttcctgcatcacaaagagaaagttcacgagtacaacgtcctgctggagaccctgagcaggaccgaagaaatccacgagaacgtcattagcccctcggacctgagcgacatcaagggcagcgtccttgcacagcagcancacggagcttgaagganaagctgcganntcanaccangggcctgggaccgccttgcgcagggtcangccaccaggggctacagnacttganggctga

SEQ ID NO: 214 - DNA, 398 bp, Homo sapiens
  misc_feature (12)..(12): a,t,g,c
gaatgacgtgcntttattggaagctattctgacatcactttccagactgtctcactgtcttgggaccaggcatgggaggcaggggtgggaatcttcttgtgattgtgggtggtggctggaggagtggagtggtgggaggcggctcagtcgcgggcactcctgcgactggcggatncggtccggatggaataagccttcaggagcccaggacccgcactgctggagaagctcagggcattgatgccatggttcccccgagggtcagcccaatgccaccgccactgctactgccaccagtggaattcatcacagagatattcacggctcccactccatctccagccaaccggttctcctcgccctccagcagcttgcggtaggtggcgatctcgaatgtc

SEQ ID NO: 215 - DNA, 237 bp, Homo sapiens
  misc_feature (27)..(27): a,t,g,c
cctacatgagcaaggtggagctggagncaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagctggtac

SEQ ID NO: 216 - DNA, 519 bp, Homo sapiens
  misc_feature (319)..(319): a,t,g,c
ttgtttgagatctttgtattttgttaacaaacactgagatccatcgaagctgtgtgggtcctggcagaggtcagcaaaggggtccttggggcctgcatcagctcagaagcccagacccctccctgacaatccttgcctcatcgctcctgtggctaagatacttcttcagccctttaacaggattactggtgtatgtgagacagccactagcagcccctgccctaccggcctctcccccaacccttctccacatacatcacccccgccaccaagcaggcagcactggggcttgattccacccctcccagaggatatggangaaagccccacatctggagagggacagggcaactgcatttctggcaaatgtggtggcatgaggctgccagtggtttgacagttcaggatctccaggtgcgtntggggccccatagcctcctgtcctccagtgaaaagatgagaagactctgctcctgggagtcctggagatctggtgactaggtacccaggcttctgctg

SEQ ID NO: 217 - DNA, 398 bp, Homo sapiens
ttcaggcagagacgcggcaccatggctagcaagaaagtctgcattgtaggctccgggaactggggctcagccatcgccaagatcgtgggtggaaatgcagcccagctggcacagtttgacccacgggtgaccatgtgggtatttgaggaagacattggaggcaaaaagctgacttgagatcatcaacacgcagcatgagaatgtcaaatacctgccagggcacaagttgcccccaaatgtggtggctgtcccagatgtggtccaggctgcagaggatgcttgacatcctgatctttgtggtgccccatcagttcatcggcaagatcttgtgaccagctcaagggccatctgaaaggcaaacgccacttggcatatctcttaattaaggggggtagaca

SEQ ID NO: 218 - DNA, 355 bp, Homo sapiens
  misc_feature (48)..(48): a,t,g,c
ttttttttttttttttttttttttttatacttcaaaagcaattttccnttattaaaagaatgcttanaaaaagtncatttcacttctgcgtcatttcttctggtcactcccagctcacttgcagcacttggccttcccnctgtaacaggtgccttgaattttggtaaagatcgggcaggcagaatagagacattgccctccactgctgacgcaattgtaatgatcagatctgcggccaaggcctgtgagaaagttaccacctgaggccatctcagacaaaagtaagcagagagtaaacagcagaaggtaggaagttctcatggcgactggcaggcaacactcaggatttcaggaa

SEQ ID NO: 219 - DNA, 371 bp, Homo sapiens
  misc_feature (37)..(37): a,t,g,c
gctcagctccaaaggagccagcctctccccagttccnnnnnncccncgtganagtnccccncncnccatgagaacttcctaccttctgctgtttactctctgcttacttttgtctgagatggcctcaggtggtaactttctcacaggccttgcgcnangatctgatcattacaattgcgtcaagtnggagggcaatgtctctattctgcctgcccgatctttaccaaaattcaaggcacctgttacagagggaaggccaagtgctgcaagtgagctgggagtgaccagaagaaatgacgcagaaagtgaaatgaactttttataaagcattcttttaaataaaaggaaaatttgcttttgaaagtataaaa

SEQ ID NO: 220 - DNA, 669 bp, Homo sapiens
  misc_feature (614)..(614): a,t,g,c
ttttttttttttttttttctgcttcacatgttttattttggtgtacattcttctagaaccaggttcatttttccagttttgtagaaaaatagatgttccagccaccttttacttaactgtctagtcttttaagaccaatcagtatgttccctggaaagatgaataagtctcatgactaattttttaaaaattctttaagacaaagaaataactttctttttttactcccaaagcacagtatctcaacagcagcagccaacatggggttttagcagcttaactttaccccctaaataaagctttgtataaaccagtgattttactacaaaaaacactgtccttgaaagaaaggagtggcagtcagacatcaatgcaaaacttggaatgattagataataaacatggcacttacaaaaggtagcttattagaatattccacttaagaagaggttacttttctgtccctccttgccccctcgaaaaacaaaaaaaaaagaaaaaacatttccttaaaaattccccttaaatgtaggtttataaaatgtcgaaatgcctgtacccaaaacaagtgcttaaaaaaaaaaaaaaaaaaacttgtggctaacaactcttanaattcngatatatatttttataacatctaagacggcagagttcttanattccgta

SEQ ID NO: 221 - DNA, 597 bp, Homo sapiens
  misc_feature (370)..(370): a,t,g,c
gagagaatttacaatgaaattccggacattaatctggatgtcccacattcatactctgtgctggagcggtttgtagaagaatgttttcaggctggaataatttccaaacaactcagagatctttgtccttcaaggggcagaaagcgttttgtaagcgaaggagatggaggtcgtcttaaaccagagagctactgaatataagaactcttgcagtcttagatgttataaaaatatatatctgaattgtaagagttgttagcacaagtttttttttttttttttttaagcacttgttttgggtacaaggcatttctgacattttataaacctacatttaaggggaatttttaaaggaaatgttttttccctnttgtnttgtttttcgagggggcaaggagggacagaaaagtaacctcttcttaagtggaatattccaataagctaccttttgtaagtgccatggttattanccaatcattcccagtttttnccttgatgtctgactggccactcccttctttcnagggcagtggtttttggtagtnaaaatccccgggttttatacaaagctttatttaggggggtaaagtttaaggc

SEQ ID NO: 222 - DNA, 203 bp, Homo sapiens
gatattaataatcaagttcaaattttaatttttcatctgctactcaattttaaaagagaagtcactctcagtagtgttcccaagtgagcgattgcaccttctatgatgtgaaaaagttgctaaagatcctattgccattagccgttcttaagtgctgaacgtaactgagagaattgtttgcaatagacatgctctgtcactgg

SEQ ID NO: 223 - DNA, 418 bp, Homo sapiens
attattgacaacaactttaaatactggaaaggactggacgaaatgaagctgcggaacctccgaccacctcctgaatagtgggagacaccacccagagccctgaagctttgttccttcggtcatttggaattcctgagggcagccagagctccttggtcctttcagtactaggcagaacagcccccgatctgcatagcctgtgaaagcccacggggacatcagtaaccttctgcagccaccatccaatgccattactgtcaagtgagacttggccactgtagcctgggcctgctgcaggagctcttcagaaaggcacatgaggaccacggtttgcctcagtttctggtaaaacacaaggtctggagtgcccctgcaaagggtattgatggacttcctgccagtgacagagcatgtctat

SEQ ID NO: 224 - DNA, 392 bp, Homo sapiens
caagatccaaagaaaaaattttatttacaatagagaattttatttgaaacatgcatttcttgtttttttaaaaacaaatcagcaaatgcagatcaagtttacactccttaaggcaagagtccctatgcacgctgtacatgttcatattaaatccaaaagctgagttaggttgtccaaacaaactcaccagacaggttacataagatagtgtaccacaacaaatttaaattttgaacaaaacacatctctcctcctttggtctcacacagaaattaaagtctccaaatacagacaatactatccattctccagctcttctccagattcacgagtcccagccaggtcggcccatccacactccccgttgaagtctggtcctttcttcagtagtt

SEQ ID NO: 225 - DNA, 467 bp, Homo sapiens
  misc_feature (454)..(454): a,t,g,c
ctcttgtgtgacctgatagctccctggaactttgggtctgtgtgtgacacatgagactcacagttggagttctccagctctggaggtgctgaaggagctgcattaattctggaagacgactccatgcagcaactactgaagaaaggaccagacttcaacggggagtgtggatgggccgacctggctgggactcgtgaatctggagaagagctggagaatggatagtattgtctgtatttggagactttaatttctgtgtgagaccaaaggaggagagatgtgttttgttcaaaatttaaatttgttgtggtacactatcttatgtaacctgtctggtgagtttgtttggacaacctaactcagcttttggatttaatatgaacatgtacagcgtgcatagggactcttgccttaaggagtgtaaacttgatctgcatttgctgatttgtttttnaaaaacaagaaat

SEQ ID NO: 226 - DNA, 368 bp, Homo sapiens
gtgtgtaaagggtttttattggagagcctgtgcggctgaagtgccaccaaggatttggaaggtcactttcagcagccgccatttctgccaggaccagtggcaagcacctggcagatggagcccgggtgtttctgcgtaaggcagaggaatccagcttttccatgagattcagctgcagttgtcgaaaaccctgtgtgagccagcagttccagttcaaaggttgagggggcgaacagctgcgaggtggccaggctcccgtgagtcaccactcaggcctgagtacaccgtggagaggagagataaagcagccacggctgttcttgttgccagtcccacccctctggcaaacagatgggggaaaacagaga

SEQ ID NO: 227 - DNA, 426 bp, Homo sapiens
taaagatgagattcattccttttttgaaagaataaagactgagaattataggtaagaggggatagaaaaggaaaatggataagaattattaaaagttatgcaaatctctaatgtgaaaacttgggttcaaggaaaattctgtttgagccagtggtttaatttaagaaaaattaaaatctgcagagaaaagctttcctaatcatcctgatataaattactcaacagcactaagctgttcataatgtaccaggctgagaaatctgattgcagagaaggaaaaaaaaaaaaaaagacttctaaaataacgtcacttcttcagctttaacatttggctgtgcaccctttttaaaaataataccagtttttttccagcagtttacacctacagtatgcaaacaaatatcagatgtttgtctcagtcagaaa

SEQ ID NO: 228 - DNA, 445 bp, Homo sapiens
ctaggagtagctggtaaaggcacatagctgtccctcatgtcctgagctctcagaggacttggtatggaatgctgcctatgcctgtgaactttgtaattagaaacaggttcaagttttggggttttctttttgccagatactagctgtgtgattttgagcaagttaacctccctgtgcctcagtttcttctgctgtaaaatgggggtgacaatggtccctataccttaggacagtgagctgtcacatgaagcccttaacacagtggctggaatgtgataagggatattaacttgtagataccttgtggagtgaaacattgaggtgattttgtagacaacaagaggagaaggaaggtattttaaaatccaggcgctaccctcatgccgactaaggtgtgagaggctcacagctcacatgagagggtgatcttgcctgaagcacatga

SEQ ID NO: 229 - DNA, 391 bp, Homo sapiens
gagaatgacaagagcttccgcggtgtcattctgacctcggaccgcccgggtgtcttctcggccggcctggacctgacggatatgtgtgggaggagccccgcccactacgctgggtactggaaggccgttcaggagctgtggctgcggttgtaccagtccaacctggtgctggtctccgccatcaacggagcctgccccgctggaggctgcctggtggccctgacctgtgactaccgcatcctggcggacaaccccaggtactgcataggactcaatgagacccagctgggcatcatcgcccctttctggttgaaagacaccctggagaacaccatcgggcaccgggcggcggacgtgccctgcagctggggctgtcttcccgccggcgagg

SEQ ID NO: 230 - DNA, 459 bp, Homo sapiens
ccttaagccttttctgttcccttcaggacctaggcttttgaaacccaaaagccaggaaaacatgcctttgttatctgctttctgcaatcacgtctcttccatggggcactgagcagagaatggtgtggccaagtgagtagtgagaagcagtgaggaggtgtgagctaggtgtctgttcccattttagaaaatactgttcctacatcagaaataccacattaagacgtatagagccaggtcactgggatgcttgaaccaaatagctgggattctggacagagtcagcagagtacagaaggctctgaagtgggagacggagctggggtgcatccctcccagtgaggaggggtcatgaggggcgtctgggaagagggacatttgaactaggattagctgagttgccatgatgctaagataatgggagagtgttctttgtggtcaccagtgtccacatggcat

SEQ ID NO: 231 - DNA, 413 bp, Homo sapiens
cttttaatatgacattcttttctaatattgtaatcagaaaatacagtatagccattttcatttgacacaaaaaatattcagggtgtaaaaataacaaaagactcattcttttgtttacattattttgtgccacattttaaaatgtcttctgttactgcaaactattttacagctttcaaaattgactagtaattcttgacattttaaatacaaaattgtcctaaacaaaaccagcacgtattcaatatgatctataagttaatctttggaaagacatttcgttttctaatattgacaattttacacaggtgatttgtaacttttgaagttctggctgaactcagcatcttttttcaggtgtctaagaccttcttcagagaagactcctagaggatatatttaagacccaactc

SEQ ID NO: 232 - DNA, 514 bp, Homo sapiens
gaaaaaacttctttttcttggcaattgcttgggatcctgtacattgcccttccacttgtgagggaaaaaaataatgacacggacatcaggtgtgttgcagtgattcacagtcatccacagaccctcaccaccatctccactggaaaatattgaacactcgccactgctagggtagtatgtagtacatgaatggcagacaccggcgagatctcaagtgttacgacggatgaaaattccacctgttaagtctcctttccatccaagctctagcagttggttgttccatgggtgtggtgaggctgatctgattacatgcttatgtaaaatccaaataaagccccttgaatggaaaacaggttaatttgggaagtcactagtaaagcaggttaatttggtaagtcactagtaaagcactaggctcatcaggagttgggtcttaaatatatcctctaggagtcttctctgaagaaggtcttagacacctgaaaaaagatgctgagttcagccagaactt

SEQ ID NO: 233 - DNA, 327 bp, Homo sapiens
cttgtatgaatcatgacaacgtctaaatctttactattcttctggcaaaagcatcagtaagaaagaaggcgaaaaagagaagtatagcctttatgtcagaaaaacattctttttagctgcttactttctcatgaaaagtaaagatgtttacagtgtatgccaagttttcagtttctgtataacaacaggtagaggttctaatcatattgaaaattgtgttataatggtctgagccatgttgctaggaaacaataggttccaattttgtattcctgctctcctgtgctgaaaagtgactggatactgtacaggttcatgttctctggc

SEQ ID NO: 234 - DNA, 345 bp, Homo sapiens
aacagtaattcttcagactttattaaaaaatgacataaagtgcatcttattaaaaaatgtataaaaaccacataaattcagggcccctgtgctgggcagtgttgatatcccttagagtggaggaaggtgagggatggagggtgaactggggactggggagaggaccagggtgcagttagttcctcgtgtttgagttcaaagatggagcgagggtggatatggtgggaaggggcacacgggttctcacgcaacaacggaggaaggcaggcgacagtctcttccctgaattctgagggaaaggcgtacattgtcacgaaatctctcctgagctcgcgctgtcctctc

SEQ ID NO: 235 - DNA, 340 bp, Homo sapiens
gagggaagagatagcatggcatgcagcacacacggctgctccagttcatggcctcccaggggtgctggggatgcatccaaagtggttgtctgagacagagttggaaaccctcaccaactggcctcttcaccttccacattatcccgctgccaccggctgccctgtctcactgcagattcaggaccagcttgggctgcgtgcgttctgccttgccagtcagccgaggatgtagttgttgctgccgtcgtcccaccacctcagggaccagagggctaggttggcactgcggccctcaccaggtcctgggctcggacccaactcctggacctttccagcctgt

SEQ ID NO: 236 - DNA, 329 bp, Homo sapiens
aaatatttaattcccatttatttaaagtttcgtcagtctgtgtacactatttatattaaaaacacaggaagctggggctcctagcaaaaatatacatttcaattttggagattgttcagacactgagaagagctgtatcctcagcaccagacccgggtgggggcagggacgcggcatgtggcgcgggagggggaggtgggtcccagcagctgtcccttcatagccttggcctgaaaggaagcaccccaacccccatcagctgggtaggtggggagcgggaagaatcctgccagcagagagtgatcctggggcatggaagggaggctgca

SEQ ID NO: 237 - DNA, 292 bp, Homo sapiens
ttacttggtaacacctttttaataggtcaaagtaactgtacagttcattatattcttcctgagaataatttatatcccccaacaaactaaagggagggtatattttttcaaaattaacagaatggatggtgtaactggacatcaaaagaaaaccaaattcctgttttgcttcattctctcctgggaagacagatacaaggaagggttagttaactccagtggctgtaaaatagtcaacatggctttaatctttctttataaattatataaaaatggaaaaaggggatggttc

SEQ ID NO: 238 - DNA, 532 bp, Homo sapiens
atctctctaaaaaagaaaaaaaaaatcactgaaaacagttctcctgacctatctcttgtctttcagattgagatggcacagaagctattgaactctgacctgggtgagctcatcaacaagatgaaactggcccagcagtatgtcatgaccagcctccagcaagagtacaaaaagcaaatgctgactgctgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcacttacagctccaacttttttgaatgaccatctggttgaaaaatctttctccatatagttttaccacaccttgaattggggttcaatatttggtgtggttttttcaatcatgatgttcggaaaaatccgggttcaaaagtggcggtttctagaatg

SEQ ID NO: 239 - DNA, 516 bp, Homo sapiens
tagaaatattacactttattgaattctggagcagctaatctaccctcccctattgaccaatgacaagaatggaagaaaatatacaatggttaaagaaagagtctataaccactgtttgtttaaaatgttgaaacaactttcactgtactggtgaaacagttttaataccctaacatacacagtaatgattcattcttgtttaaaacaagaagtgatttcgacttgggattgggatttttattttttgagatggagttttgttcttgttgcccaggctggagtgcaatggcacaatcctggctcactgcaacctccgcctcctgggttcaagcaattctcctgcctcagcctcctgagtagatgatgggattacaggcatgcaccaccacgcccaggtaattttgtatttttagtagagatggggtttctccatgttggtaaggctggtctcgaactcccgacctcaggtgatccgcctgtctcggcctcccaaagtgctgggattacaggcgcgac

SEQ ID NO: 240 - DNA, 572 bp, Homo sapiens
caagttttcttttataagcctttaattcatcctcagtgactctggcaaggctgcttctctatcactggctttgcacagaagtatgctctacttgcgttgctttagggcaggattctattttgagggaaaagacagtatccttattaccttttgtttgtttaatagcacaaatgcttatttgttatccaaaaacaacctccttcttatctgtgataaatctatagaaagaatttagctgcaagtggacaaaggaacaagcccccagaaaagaaagggaagaactgccttcttatactacagaacatgcattagtgtgggctatatagctgtggctcatgctacccaattccagatttctttgtcctctaagagttgattgctgtatattaaaattgaacatcagaggatgggaagagggctctgtaagccagaaccttactaaagtagagggcacaatcagtgtgaataaattcacttcagaatctcaagtcaaggccaggcacggcagctcgcgcctgtaatcccagcactttgggaggccgagacaggcggatcacctgaggtcgggagtt

SEQ ID NO: 241 - DNA, 364 bp, Homo sapiens
ttttctgagagtcatttatttcttttgtcattgccaaacattccccaggtcttactcaccagaaattaacatagcttcttcatttctcctccctgacaaccctcatctactgaaagggctttaagggcacactgccaatgaaaatgcagagtgcatataccatgtccttaacttgtttcaaaactacctattctggcagtatccaattcaggtttcagtgctcccttgtttgaaagtggtcttcacaaaagcccacattttaaagctatttggaggagcacaaagagttaaagtggtaatagcctttcagaatttgaaaaggtagtacttgtctatatcaggttcattttttatgtggcatatg

SEQ ID NO: 242 - DNA, 579 bp, Homo sapiens
  misc_feature (10)..(10): a,t,g,c
attcggcacngagggtttgtcctctcaatatggttttaagattgaaagtaagaaaacggatttaggatgaaaactctagaactaccctatgctgtttatactgggaaatgctttgtaccaagtagcagtgactagacccacagacatgaaaagcaaccttaggagtaaagtgacccaaacattaaaatgacaggaaagagaaagtagaagcagcaataaatactgccccaactttcctggagcccgaggcctcatccatagctatgatcacttgccctctgaagcttatttttgcttctttggttttaagaattgagaaatatcacattgcccctgatgttttgacagtctcctagtgctcctggtagtgccactaagggaaaaccaaggtgcgcattccttctccctggactttaccttacttgttagtctacgccccactgtttccacccatccccttagccaacctctgtctttttgaattttctgagaatattgtcctatcctcttttatatatggagttctctcctctttatatcctgagactttgacaccagatgtagatatttatctggagc

SEQ ID NO: 243 - DNA, 382 bp, Homo sapiens
tttaaattagtagagacagggaatcttactatgtgacccagactggtcttcaattcctgggctcaagcgatcctctcgcctcagcctcccaaggtggggttatatgcgtgacgcgctgtgcccggctccaaagaacatttcttaagattggtggtgcaaggatcacaccttgagaaacactgatttaggccttcccacagtacaaagaaatgttgcctgccccatccttacagcacacctgatgacttacaagaggtgctgctgaattcctcccagggaagcaaccttaattcttctcagcaagacaaggaggcagccttcaggaaggacccaggagcttggtattagaggatgatccaagtctgatggcaaatttagagtg

SEQ ID NO: 244 - DNA, 210 bp, Homo sapiens
gggtgagcacttagccagtaaaaatctgtgctttgtataaaaagaactttggactgaggttatatgagtcttcctgcaggctggagacctctgttacctttacttctctacttttgttgtcttgagaccagcttttttatttttggtgaggagaacagcaggcagttcaaatgtcagggaaagacaagttcagcttgctttttccttatt

SEQ ID NO: 245 - DNA, 393 bp, Homo sapiens
tttgacattgtcataattttttattatgtatcaaattgtcttcaatataagttacaacttgattaaagttgatagacatttgtatctatttaaagacaaaaaaattcttttatgtacaatatcttgtctagagtctagcaaatatagtacctttcattgcaggatttctgcttaatataacaagcaaaaacaaacacctgaaaaaatataaaccaaagcaaaccaaaccccccgttcaactacaaatgtcaatatggaatgaagcattaaaagccaaccttaaagtaacttcagctttaatctagcaatgcagaatgattactaaaattagtggcaaaaaaccaaccaccaaccaccaaccaaaccaaaccaaccaaccaaaaaatcccacca

SEQ ID NO: 246 - DNA, 315 bp, Homo sapiens
tttaagaggtactcaaatatttatttaatacatgaataaatggtgagtcagtgaaaaaaaatctgaagttgtacaaaagtggaagctcagtgttccaaattatttaatagcctgtcattatcagatgatccgagcaatttatttttgcattgttacaggctgtgttctccagaagtagacagtagaccctcagatgtagtctggtgttcaagatgtttgttaaagatcagtatctgtaaaaggaaaagagttagaagcagtatcaactagaggaaaaagctaacatgcagtgcaagcccaatgaagcctctgtag

SEQ ID NO: 247 - DNA, 429 bp, Homo sapiens
cggttattggatatgtgaaatatcatactatagcagatatggaggtttgccactcagatcacttcaagaaaggacttcccgcccagcagtggggagtgcagtcaccaggcagcctcctactctcagcttcttcatagtcttagctgcagaaagccacctcactcaagttcacattcttcccagagcagcctgcatcagatgactaagcagagtgtcaggggataggagatagtagggatatggaaactcggtcatttcaacccaatgtaagaccagtctaacaggcaatgcttattctaaagctacctacagaggcttcattgggcttgcactgcatgttagctttttcctctagttgatactgcttctaactcttttccttttacagatactgatctttaacaaacatcttgaacaccagactacatc

SEQ ID NO: 248 - DNA, 184 bp, Homo sapiens
  misc_feature (133)..(133): a,t,g,c
aatgcattctagtaaaagttcatttgacagtctttttttttttttttgaaattcacagtccattaaattcttcccctctcttcttttagcaaacttgtaacatccttttatatcctttcttaacagaaggaanttaagcaancaaaccagtntttngcctttncttccccggttanactccccc

SEQ ID NO: 249 - DNA, 300 bp, Homo sapiens
  misc_feature (185)..(185): a,t,g,c
cgaactggccgcgagattgctcttcagcgccgtcgagtgggcccggaacatccccttcttccccgacctgcagatcacggaccaggtggccctgcttcgcctcacctggagcgagctgtttgtgttgaatgcggcgagtgctccatgcccctccacgtcgccccgctcctgggccgccgccgctngncattgcttcgcccatgtccgccgaccgggtggtgcctttatgggaccacatacggatttttccaagagcaagtgggagaagcttcaaggcgcttgcacgtttggatttcagcc

SEQ ID NO: 250 - DNA, 338 bp, Homo sapiens
ttttagagttgaaatatattctttattttcaggatggaaataggatagggaaggaggaaagatacctttgttagttgccactgcagtaccatcgaaagaacatccacgccaacactgtcgcactcgctgatgcgagggccccgaggactgcaagcaggaacagattgctgggcgtcagagcctcgggcctggccagcagctcgcccaggctgggcctccagggctcgatcagctcggcatcccgcggcgcccagccgagcacttcccggaacgaggcctgcaactctgtctccgcccgcggggcgcccagtcacgttaagaaagaagtagagccgggc

SEQ ID NO: 251 - DNA, 349 bp, Homo sapiens
ctggcgcggatcggccggctcactcacgcaccgcgggacgttctcctgcgtgatcaagcaagaccagcgccccctggcccggctctacttctttcttaacgtgacgggcccgcccccgcggggaggacagagttgcaggcctcgttccgggaagtgctgcgctggcgcgcgggatgccgagctgatcgagccctggaggcccagcctgggcgagctgctggccaggcccgaggctctgacgcccagcaatctgttcctgcttgcagtcctcggggccctcgcatcagcagtgcgacagtgttggcgtggatgttctttcgatggtactgcagtggcaactaaacaaagg

SEQ ID NO: 252 - DNA, 493 bp, Homo sapiens
tttttttttttttttcccgtggcactcaatcttttattttcttaaactgtacacaaacgtacaatactttaacagcaagtctatgggaaaattgtttggttttaaattattatgaaacagaatctatggggagctttattaaataaacataaagatgaggatttaaggatacacacctgcattctatcatagtcatttttactctaccttctgggtgtgtaaggatggaaaagacacatcaaaccgaataaacaaaatccattcataccctgaaacgttggcaggccactcaagggactgctcagaacgtccacctcatctcagatggcctcaccgtctaataaaattaaaactgatctgttggcctctttggttccaaaattatgtataatacatttaactgtattctcttttttttttttttgctgctataaaataactttttttcaatggcagttctgactaatctgcacttaatcagtgcaacataaaa

SEQ ID NO: 253 - DNA, 227 bp, Homo sapiens
ctcgctcgccctctagatgtccaagtgccacgtgaactatgcaatttaaagggttgacccacactagacgaaactggactcgtacgactctttttatattttttatacttgaaatgaaatcctttgcttcttttttaagcgaatgattgcttttaatgtttgcactgatttagttgcatgattagtcagaaactgccatttgaaaaaaagttatttttatagcagca

SEQ ID NO: 254 - DNA, 419 bp, Homo sapiens
cccatgtccagttctgctctgacttgtgggtccaccttagaaaagtcaggagacacctgggaaatgaaggcactagactcttccagactcgttccatggccacccagaggccttgggtcatccacccaacatcccaacaaaccccactgtgcactggcatcatgccagggtccaggtgtcctgccaggagcagcctctgccctcccagagctgacatttcagggggatgtgtgccaaagtgagacctgtcagagatatttacaagcagccatctctcttgacatcgctgtatcccaaataaatcttctgggaagaccctcttcacctccagctctcctgatacagcaaggcagttgtgagcaagttattcataactctacacctcaatttcttggtatggaagatggggataatgagag

SEQ ID NO: 255 - DNA, 331 bp, Homo sapiens
ttttttttttcagactttgtagctgtttttattattaatattatcttctcctttaagcatccctttaagggtgaaatgaaatccaaccatttacagagaaaatgatttcagaatgtacatattgattttcccttacaaaaaaaataatactattattgatgtccttacattatgcatttcccagggtcctgccttctgcttggacaagtcccgtctccccacccagactctccctgttgcccccaaagatctcccatgctgcccctgggaaccgacgtgacccatgggtggataaggccagccatgcttcccggatgcttggcatggacag

SEQ ID NO: 256 - DNA, 388 bp, Homo sapiens
cactgctgccatgtcccatgtccacctttctccccgggaataactggccctgagacccctagacccaaggaggcctgtccatgccaagcatccgggaagcatggctggccttatccacccatgggtcacgtcggttcccaggggcagcatgggagatctttgggggcaacagggagagtctgggtggggagacgggacttgtccaagcagaaggcaggaccctgggaaatgcataatgtaaggacatcaataatagtattattttttttgtaagggaaaatcaatatgtacattctgaaatcattttctctgtaaatggttggatttcatttcacccttaaagggatgcttaaaggagaagataatattaataataaaaacagctaca

SEQ ID NO: 257 - DNA, 385 bp, Homo sapiens
tttttcaaagaaactccacatttattgtctcatctagtcattatcataacacagcaaggtgggtggggatggttacatttttctcattctacagctctggatttgtgggaaataaatgcaccatcatcctactcaccttggaaaaatgaaggctgtggggatgcataacaatgataatagctaacgctcatgaatgcttaccacgtgctaagcacttttctaagcattatctatgtattaatccaattaatccccacggtcactgcatcaggtagcacagcagttatttgtgctgttcaccaaatacttccaggcacatggcagaattgcacttccctgcccccttggagtgaggtgcggccatgtggcttgctttgactgatgc

SEQ ID NO: 258 - DNA, 420 bp, Homo sapiens
ttactggggacactgggctacatgaagtttgggtcagacacccaggccagcatcaccctcaacttgcccaattgctggtatgtcctgcccacctcaggtgagatagggagagacactggaactgttctggttgtcatagcagagagcacagcaaagctgagccatgaagctggtaatccatcactggaagtgacatatgtctctcctgctcacactgcatcagtcaaagcaagccacatggccgcacctcactccaagggggcagggaagtgcaattctgccatgtgcctggaagtatttggtgaacagcacaaataactgctgtgctacctgatgcagtgaccgtggggattaattggattaatacatagataatgcttagaaaagtgcttagcacgtggtaagcattcatgagcgtta

SEQ ID NO: 259 - DNA, 278 bp, Homo sapiens
ttttttcagaggcaaataaaaaaacacttggatttattcatgaggttagagtcatctggtcagttctactgttctcagctgatcttggtggggcttactcaagagtggtcaacagtatttgggccaggcagctctgcagatcagaggagggtgcttacatgtctggaggcttgactgggatgaccaggctgattcagcgctgttccacttgccgccttcttcagcagtctgtcttaggcatgttctcatggggaaggcttttctccattctgaagagg

SEQ ID NO: 260 - DNA, 340 bp, Homo sapiens
caggtccctggactttgggactcattctgctcgacttgatccccgctctgcccattgatcacatacacacgtttacacctggcagaagaggaagaggaggcatgaaggaatggcctcttcagaatggagaaaagccttccccatgagaacatgcctaagacagactgctgaagaaggcggcaagtggaacagcgctgaatcagcctggtcatcccagtcaagcctccagacatgtaagcaccctcctctgatctgcagagctgcctggcccaaatactgttgaccactcttgagtaagccccaccaagatcagctgagaacagtagaactgaccagatga

SEQ ID NO: 261 - DNA, 446 bp, Homo sapiens
ttttttcgagtactgtaccgtatttattgattctgtaagtttacaagatgaacagtcggtctaagatggcagcaactgcagctgcagacctcaatcaatggtccatatcaagcaattcaactttttcttatatatcacgtcatgaattttccctgcttcttggggaaacttccagcatgactagtggcgctgcctttgtataagtcccatgtcacttggtattgcacagctgtgtgtgatcccaggcacacccacttcacttcacccaagcatgcccatctagtaaggaagagatctgcatttgaatccaggtctgtctttctccaaagctgtctttactctttctctaggtaggaaattagtgagctgggcttcgattttccacagtagctgagagggctttatgaactgactcccctcctctggggcttcgtgttctcatcagc

SEQ ID NO: 262 - DNA, 477 bp, Homo sapiens
ctgtgtagtcaagagtaaccaccagatgggaagagaatacagatcagcttgtgcaataaagaaaggaaaactccaaaggagattatttccatagactcatcaacttctaagcaaggaatctcaaaaagacaatctaccagagaagtgacttggaccaaagagtaaacgaacaaaattaatgatgtggacagccctaggaatcagcttagaggttcagcctggcccctggtcggcacattctacgagctcgtggaaaagggcaagcacagactatttacagccgcagagaaatggcctcataaaaatgcataaggaatgatggaggatatttgccatctcatttttgaagcctgattcaccgtggcccttggggctttggtccttcctaatgacatctggtctactgccctttgtgctgatgagaacacgaagccccagaggaggggagtcagttcataagccctctcagctactgtg

SEQ ID NO: 263 - DNA, 361 bp, Homo sapiens
aaataagacatgccataagtcgtgaagttaacaaaatataagcatccgcacagaatatattctaaggtgacttcatttacaccgcttctcagagaaacacacaagtaaccttttgtctgcctatcagccagtgttgaaacagctttggaattcacatggaaggctgccgggctggttccccaacactagcctgatggagtcctgtatccgcaccgtgccgtcaaactggctggtttccactagaaaagcaatggagagtcagctctcccttctttacccagcgttcaactccacactgcaaaaactgcataacctgaaccctgctgtgatggcaactgaagtttaaacccaagtttggtga

SEQ ID NO: 264 - DNA, 511 bp, Homo sapiens
  misc_feature (152)..(152): a,t,g,c
gggccgagctgtccgacgtgtcactgcagggacccgcccggggtgggtctcgggctctcgctaccggagagggaggagaagggggaggttaaaggggaaggaccccggaagtgccccctcctcagtgcgggagagggagacgccgggggcangtccatgcctcccgcggcgtggttggtgcgtcccaggtgacgtcagaagcagcccgcccctgcctggatggtgcgccctgagtgacgtcaggagcagaggccggagctgtccatcagcaccaaaggccgcgggcgggctcagggcatggggccgcggttctggggcggccgagccccggctcctgcgccttccccttcctcaggcccagcccgagttcccggacgccgcgggactggagtgccagccggtgttggacgtggagcggcgccgccaccgcgccgacaccattctctccggcccagcagcccccttcctcgcacgacggactttccctggaccccagtcagttggagcctct

SEQ ID NO: 265 - DNA, 519 bp, Homo sapiens
ttttttttttttttttttcagtttggcacatttataaaagatgtaaaacagagtggggtgggggagacagaagcatgaggtgtctaggttggcatgacagacaccagacatgcaccgtgcatttaaacccctccccaactcccttcccaacccagcccagagcagcacccccccacccccatctcctgccactgccctcaggctggtggctttacagaggccaggggaccagggtgagggatgctacagtttgccccatagacaaggacaagaacccttagatgagtctggctgcgaggcagggcgatgggaaggaaggtagcccctccgcggagacatgcaggaggtggcccatgggaccttggcagggagtgaagaggtggggctgagcctggagaaaccctaccagtcagaaatctaaggcaagggtcccagcgtcccctccaagcctgcccacccccacacacatacacacagacacacgggcaacacactcacaaccctgagcacaccccaatg

SEQ ID NO: 266 - DNA, 218 bp, Homo sapiens
ttaaaggttgagacacgtctaaccagtttaatgacttcgaaaccgtgcaaatgccaaactatggagcactagggatacaagaggcaccaaggcctggggggtgggggtgggggacactacaacattgtcatggggaaaacgggatcacctaatattggtaggggaaaagggcggtccactggcagctcagaactatgacatattcctcaggggagcct

SEQ ID NO: 267 - DNA, 237 bp, Homo sapiens
agctggcttgctcctactttcaggaagggatgcaggctcccctgaggaatatgtcatagttctgagctgccagtggaccgcccttttcccctaccaatattaggtgatcccgttttccccatgacaatgttgtagtgtcccccacccccaccccccaggccttggtgcctcttgtatccctagtgctccatagtttggcatttgcacggtttcgaagtcattaaactggttagacgt

SEQ ID NO: 268 - DNA, 551 bp, Homo sapiens
gcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccatggctctaatggagatagagagaggttgcagatgaagataagaga

SEQ ID NO: 269 - DNA, 518 bp, Homo sapiens
gttaccaatctgaagtgggagcggccggggttttttttttttttttttccttaacagtctcaggtatcaaccagaagaagttgctgatgacccatttactgatgattttcgaggtctattggcaaaagaagattggtggttaccgctggggctgttgctggttccattcatagtactggaaatgtgaggaaactgtggatgaggagactgcactggagtactggggctaggcaaacaagaagaggtggagggaatacctcctgctgggctgttggccttgtcactcccagagtcactttccagttctccagcatttgtcagtccatctctctggtgactgatcttcattcttttacaagtaggtcgaactctgtatttcaatggaagtggaccattccttctccaggtataaatgtaggcaatatcaattagtgtataataatcctttaaaggtccccccatacatgacacaatctggaaagtttaaggatgtcatttacttccggaaactttctaag

SEQ ID NO: 270 - DNA, 379 bp, Homo sapiens
gggaacgtgaattttaatgagggggcagaccgaggaggtggtggctgcccggagatcagggccaggctgtgctagatggcgcctggaaggggggtcacccaagtctccctgctgtcatttcaggaggccgacccaagtctccctgctgtcatttcaggaggccgaattttttcccaatcccagagaaggtgtcagaggcctggttagcagtcttgtcgatggtttcctgggtggtcttggccagctggtccatggctttctgccccgcctctgtggcctggtccaccacttgctgagctgccgctccggccgctgacacggcttcctgggcggtcccctccacctgttgcttcaggtcctgcaagcacttgcttgccat

SEQ ID NO: 271 - DNA, 465 bp, Homo sapiens
tttaaataaacattttcaaggtttgtccaaaagaaggccatataggttcttggctagcggaagacaattcagaacagctgttgcacacttggactgtcaccttctccaggctggcagttgatatcttattttttttccaactcatttttattaaaaaaataaaaaaatgctccaactatcagctttacaaaatctctaagggaaacacaagagcaaggtgctgaggtaaaaacacctgaggtagcttcttctgtgtgtttttctcgttaaaaaaatctgtgaatttaacgccctgggccaacaaccttggtaaatttctactttcctccacattttttttttttaaagaaaggaatcattttgctgaatattgatggcttatacaccaaaatgcaaaaagacaaaatacattctttcattgtggaattttttctttgtttggttgattggttggtttggtgggtt

SEQ ID NO: 272 - DNA, 343 bp, Homo sapiens
tttttttaacttgaatccaaatgatttatttcagttttggctcttaagccacttccattgggaagaagaaactcccaggagtgacctttgagagctgagggttctcggggtggggcagtgtgaggggctgggccagtgtgaggggcaaagtccaacgggcatcccactcctgcggcctgggctttcccagctgatggggtgggggacacccatttctttagtcccagcccccagggaacggagcagagggcagagggagtgaggctgcacccaccaggaagcctgggagtgcccttgctgggggctggggcagaggcagcccggcaggccctacagggaggct

SEQ ID NO: 273 - DNA, 477 bp, Homo sapiens
tttgatcgcccaagcagagactgtatttcccagattttctggcagccagagttggtcacagggctaattctggtccatggaatgtggctggaaatgatgtgtccaacttccgagtcacgccctgcaaaggaggttgtttcccctccattctgcttttccatcctcctgctggcttggaagtggacggtcctggatcagctgccctgtgacacactggcgatgaaagcagtctcctcaggtgaacacattttatgttgcagtcacagtatgagcttcagattgtcttttaaaaatacttttccttcttccatcatggacccaggaacacgccagcagagaggtgtctgaaggcagcgctcaggtcgcagaagcatgaggatctggctgtcaccagccccgtagccagggctgtgaggaccagggcagccgctgtcccctgcctagcctggcagcctgggatgaaggaggacaccaggc

SEQ ID NO: 274 - DNA, 258 bp, Homo sapiens
tttgaaatttggaacttttattaaatactcattcatccatgtgccaccaccacccagagcaggaaaaaaattatcaaaatggaattaaaaaaaacaaaacaaatcaaacccaatccccagcagtctatgtacagggccactccctgcctctctgccatagagaggttggggggcagctgaggagtggtgggggctgggcaccttttcttcagccacaggcccctgaggaattaattgactgtggtgtcaggaagtccc

SEQ ID NO: 275 - DNA, 179 bp, Homo sapiens
ttatttagaacttgaaaactttatttcagtaaatggaaaaaaaataatgcatgtagctttaacagttcacaagaagaaaatagaggacacaaaagaacaccttgagaggcatctgtgtaagaagcagctgaaatcattcccagggtaaccccccatcccataacaaacggatagggaga

SEQ ID NO: 276 - DNA, 395 bp, Homo sapiens
taacaaaccttttttttttttttattggagataaaaacagcgaagtcccacataccataccctacaagacacaaggtgcgcagacgagccttggctatgtaccggcgctgcaggaagaggctgtccgccgggcctgggctgctccagctacgcgaggaggcggccccattgcaaagtgcagtttctccgcggaggtggcggtgggtcagtggcagagggccatggtttccatgttaaggaagcggacgtgcatcttggtctcaatgtcgatcccctgccagatcatctgccccatgcacacgctggccgcctccatcatggccccgtcggcgatggagcgagcggactccttctcgatgtgagggtttcccgacagcagctcctcgaccacttta

SEQ ID NO: 277 - DNA, 406 bp, Homo sapiens
attacaaagggcttatgatgattttattggctgccagaggaacacagtaaataactcccaggtgtcttgttggaaattaatcatgtggaaattatcacttctttggtgcaataatgccttccagttgggccttcagctgctgatttgttcgcttcaggaactgaatctcctcattgtgcttcttctcctccacctgccgcctctcgctctcccgcttctcagtggcttcacattttgccttctgctcgttcacttgcctctccaggtctctcttttccgtctccaattctgcgattttcctctccatgtctgacttcccctgctcagcctgcagtgccttcctcatgccaaacgccacgctgctctcgtacagggtctggtaggcagcgatggtcatgcggatctc

SEQ ID NO: 278 - DNA, 495 bp, Homo sapiens
ccgcccgcagactctttgctcaagtacgacaccccagtgctggtgagccggaacacggagaaacggagccccaaggtcggctactgaaagtcagcccccagcagcctggaccttcaggttcagccccacagccacccaagaccaagctcccctcaactccctgtgtcccagatcctacaaagcaggcagaagaaatcttgaatgccatactacccccaagggagtgggtggaagacacgcagctatggatccagcaggtgtccagcacccctagcaccaggatggacgtggtgcacctccaggagcagttagacttaaagctgcagcagcggcaggccagggaaacaggcatctgccctgtccgcagggaactctactcacagtgttttgatgagttgatccgggaggtcaccatcaactgtgcggagagggggctgctgctgctgcgagtccgggacgagatccgcatgaccatcgctgcctaccagaccctgt

SEQ ID NO: 279 - DNA, 470 bp, Homo sapiens
  misc_feature (6)..(6): a,t,g,c
caggtntatnttntttaattatcactcacatatttcacaggaaaaggantntagcaaatgggtcaaggtggtntaaaaaaaaaatccaggtttntacatgtctctctgtttacatctgggagaaaggttntcctggcatcagtcgcagcagctgcacttctctgacgcccctttgcaaacacagccctgggcacacttgctacagcccacggggaggcaggagcagcagctnttnttgcaggagggtgcatttgcnctctttgcacttgcagggaaccagcgcagggtgcagggagacaccagcgggcgcagggagcagttggggggncccattgcaagcccgagggagagactgggacttttcccaaggagagaagcgaaggaagccagtggggggcagctcgtgcccganttccttcagccccggggggntccccctagttctaggagcggnccccaccgggtgggat

SEQ ID NO: 280 - DNA, 321 bp, Homo sapiens
  misc_feature (138)..(138): a,t,g,c
gtgccccacatgcttctttcgcattctctcttggaaagtccagtctctcctcggcttgcaatggaccccaactgctcctgcgccgctggtgtctcctgcacctgcgctggttcctgcaagtgcaaagagtgcaaatgnaactcctgcaagaagagctgctgctnctgctgccccgtgggttgttagcaagtgtgcccaggggttgtgtttgcaaaggggcgtcagagaagtgcagntgcttngacttgatgccaggggaaactttttttccagatgttaaaacagagaganatgttacaaantggggttttttttttatta

SEQ ID NO: 281 - DNA, 321 bp, Homo sapiens
tttttgacagtcataaatgcataaagttactttatttttaagtatacatataattttgaaaaatataaatgctaaaatatctcatcttcttcatctcttaattccataaaagtcacaatagaaagcttcataaaactgtaaaatactatgttataggaattttaggatttatattggaaaaagttattagtaatatattgaaatgttttctgttctttatctttttttgttccaccatgttgatctacttcatttaaaataaaaagatgcatcatttctctctccaaagtttaataaagacttaagagaataaaaataaga

SEQ ID NO: 282 - DNA, 388 bp, Homo sapiens
  misc_feature (367)..(367): a, t, g, c
ggattctttaatctccagctcagcttcaacaattcaacgctgttctttctgaaaaagtacacatcgtgccttctctacttcgctcttggaacataatttctcatggcagcttttactaaactgagtattgagccagcatttactccaggacccaacatagaactccagaaagactctgactgctgttcttgccaagaaaaatgggttgggtaccggtgcaactgttacttcatttccatgaacagaaaacttggaacgaaagtcggcatctctgtgcttctcagaaatccagcctgcttcagcttcaaaacacagatgaactgggattttatgagctccagtccaacaattttactgggattgggantctcttacagtgaggggcaca

SEQ ID NO: 283 - DNA, 155 bp, Homo sapiens
  misc_feature (5)..(5): a, t, g, c
accgngaaaactaaaagggngtnggggaaagacancttnggactgcttctctcagtncccgctgncagacaatctgaagnacttttaagngtaaccngcncactaaccctcagcctgacacccagctatgtgncgttgtcttgacactgtgnggg

SEQ ID NO: 284 - DNA, 451 bp, Homo sapiens
  misc_feature (1)..(1): a, t, g, c
naaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggnacatgctcagcagacatctgcagttaacgggcgtcccctgccccacacggtaagcaccatgccccacacctcgggtatgaaccgcctgacccaagtgaagacacctgtacaagtgcctcttgccccaccccatggcagatgagtgcctgggggggttaacttccttccgtgaggcagctggcaatgggttatgggcaggatggggcctttttccaccaggagaagcttcccaagttgattttggatgggctttttttt

SEQ ID NO: 285 - DNA, 471 bp, Homo sapiens
gtagttcaataatattttattggcaatagcataggagaaattcaatattgaatctcagaacaagaagaagctatttacaatgcatgtcaaggaagagatgggagaaggaatgtcacaaaattttttggtaaatacatattttttatagagaagtaatccatgaacctgcaacatggatagcttatccaaccaactttacaaattactattaatataagttacatgcttgccatctaaagtaactaaacccatagactgaaaaactatgtgtcaaggtaacgtgagcactttaatcactttacttatattttctaaaggcagtagtttcctctccttttcccgctatcatattagatgaagagacaagttccttcaacacaaattctgatatcggctattgtgagaatccctgtgcgagtcagctagaagcccctgccaccagtctagtgccaaccaaatgaccagatggga

SEQ ID NO: 286 - DNA, 472 bp, Homo sapiens
  misc_feature (7)..(7): a, t, g, c
ctgcgantgaccaaaataaaggcagatcccgacggttncgaggctcaggcggaggcgtgttccggggagcgcacctaccaggagctgctggtcaaccagaaccccatcgcgcanccctggcttctcgccgcctcacgcgaagctctacaaatgcatcaagaaagcggtgaagcagaagcagattggcgcggggtgaaagaggttcagaaatttgtcaacaaaggagaaaaagggatcatggttttggcaggagacacactgcccattgaggtatactgccatctcccagtcatgtgtgaggaccgaaatttgccctatgtctatatcccctctaagacggacctgggtgcagccgcattccaaagcgccccacctgtgtgataattggtcaagccccatgaggagtaccaggaggcttacgatcgagttgcngggaggaggttgcattccttgcccttaccctnttgagggg

SEQ ID NO: 287 - DNA, 430 bp, Homo sapiens
  misc_feature (311)..(311): a, t, g, c
tagtcttagcatttactttccccaccccacattcttggaacagcctttagttctacaggaaatggcactgatggacagaagactagcattaccttcatgaaagggctgttagagctgcctgggaagaaggcgtgccttggggaactgggaagatgccgtcagtgtgggtgggcaggaggacagccagtcgtcctgctgccagcccaatagcttccagcggtcaggtgcccaggtgctaccggagcccctcataggggtaaggggcagggactgcacctgcctccagggcactcattcgtaagcctcctggnactcctcatggggcttgaccattnatgcacacaggtgtggggngctttggagccttgcgctnggcancccagggtnccgttttnaggaggggatatgaggacatnaggggcaaattttc

SEQ ID NO: 288 - DNA, 561 bp, Homo sapiens
  misc_feature (554)..(554): a, t, g, c
ttttaaaaagatacagtctttacttttattgaaataccaaatattgactatgcaagctatactggtaaatgtgctctttgatgttgacagaggagggctgggctgcctgtggtttcctttcagattgtctgtgagtctaatttctcatggtcgtgtgacaaggaccccatctttagctgaaattttcaattgccaggaaagaggtagaaatctcttgtcaaggttgctcgttctatggtgggcatctgggctttagctcgtggaacttcaaatgatttctgtaccttccgaaatatttccattaggtggcagcagcaagaatatttctggaagcatgtgatgagttccgtaatgaagatggagccccttgtgcggtctctccaggacacgttatgtggtgttgaagaacagaaagcaatgaagtccttctcctccgtggatcttgcaaacagaatctgcctccaaggttctcagaggacttgtgaagagatgacngccaaaggatgctggagagtcttggacccagagttccccanggttttcaacctctgcangccccgg

SEQ ID NO: 289 - DNA, 578 bp, Homo sapiens
  misc_feature (545)..(545): a, t, g, c
gaaaagaaaaaatattatgataccaaaattgaagacaaggccctgatcttggtagactctttgcgaaagaatcgcgtggctcatcaaatgtttacccaaacacttctcaatatggaccaaaagatcaccagtgtaaaacctcttctgcaaatcgaggctggaccacctgagtcagcagaatctacaaatatactcaaactttgtcctcgtgaagaattcctgagactgtgtaaaaaaaatcatgatgagatctatccaataaaaaagagagaggaccgcagacgcctggctctcatcatatgcaatacaaagtttgatcacctgcctgcaaggaatgggggctcactatgacatcgtggggatgaaaaggctgcttcaaggcctgggctacactgtggttgacgaaaagaatctcacagccagggatatggagtcagtgctgaagggcatttgctggccagaccagagcacaagtcctctgacagcacgtccttgggtactcatgtctcatgggcatcctagaggggaatttgcggaactgggcnttaaaagggaaaaaccgggatttttctgctttt

SEQ ID NO: 290 - DNA, 469 bp, Homo sapiens
  misc_feature (454)..(454): a, t, g, c
ctcaaatatccaattttattttatcattctcgcattgtgggatgcgatctgcagctaggatcggaattcccaggcctatagatttttaaaccacaccacaggggtaaaccttaaaagaagtgaaacctaacactatatatatttccatttctaaatacagtatattacagaagtttaaatataccacctctgtgtacttacaactataaaaagatacaataactctaccaattataaataatgtagcatttcatattaaagacattatcgtacaatggaagaataggaaccctctaacgtatcactatcaaggttagtgtctatatctacttgagataaaatactgaaaattcagtgtatgaagccaaatcctgatttaacaagttattgggtagtataagtggataagtgttagccggatgaagggaaggccaatggtgggtaatttatatcncnggacaaggggtga

SEQ ID NO: 291 - DNA, 456 bp, Homo sapiens
  misc_feature (178)..(178): a, t, g, c
gggattctatggaggacctcggtcttcatccaagtggcctgagtatttcactggcaggttgtgaatttttcttttcctctttggggatccaaatgatgatgtgcaatttcatgttttaacttgggaaactgaaagtgttcccatatagcttcaaaaacaaaaacaaatgtgttatccngacggatacttttatggttactaactagtactttcctaattgggaaagtagtgcttaagtttgcaaattaagttggggagggcaataataaaatgagggcccgtaacagaaccagtgtgtgtataacgaaaaccatgtataaaatgggcctatcacccttgtcagagatataaattaccacatttgccttcccttcatcagctaacacttatcacttatactaccaataacttgttaaatcaggatttggcttcatacactgaattttcagtattttn

SEQ ID NO: 292 - DNA, 447 bp, Homo sapiens
  misc_feature (351)..(351): a, t, g, c
taaattaaaaaacaatttattgaaaaagagtaatgctttatacaaattcctattataaaaccccaaaatgtctattggtctgtttccaggtgtggtagaagaatataaaaagatcaaaattggataaattctattgtaacaatttcgttggtcattttgggccataaaatttttttgtaatgtttggtaactgatatccacatggaattacactcacacatcatgaagatctatgtatgtggcaaaagccatttaaattttaacttccaaaagcatatattctcaggtttggaaggcacactaaaatttattaggtccaattcctcataagacacagtggctgactttccntgtgtagtttattatgaagtaccatttccaaactaactatcctagcagcgtccgcaaggctaacagcaagagctctgagggcatcactgaacagat

SEQ ID NO: 293 - DNA, 330 bp, Homo sapiens
tttaaaatctcatctgaatgcatctaagtcaagcatgagggaggtgaagtatgagtcattccaaggcaaaattcttcttcagttgtgaagcatggaaccagacaagttttgtgcttccaaaatgcagtggtggtgcaggcataggatagacgttccattctggaggctagatggttgagatccaggtgtcagtacggttggctccttctgaggcctctctcctttgcttgtagacagccattttctctgtgtcttcactggtccttctctgtttgtgtcctaatctccttttcttataaggacagcatttatattggattagataccacc

SEQ ID NO: 294 - DNA, 417 bp, Homo sapiens
gaccagacagggcttccatccagcagtgcccatgcctagatatacctggcgcaccgcacaacatcccagcccacacaaggccctgctacctccatccaacatactgctagactgaccctactgaatccacattcccacatattaccctgactcatatcacctagatcatgacagacaccagatgtggccttgaaccacacctctgaacctccaatctaccattctctcttgcctcctgactccaggcagggggctcacaagttagcagagcagggcctaacccaattacatagccatagactgctatgggttgtgtttctacagaagtcaaatgttgaagtcctaacttccactatttcagaatatgactgtttttgagatagggcctttaaagaggtgattaatgctaaatgggat

SEQ ID NO: 295 - DNA, 427 bp, Homo sapiens
ttttgccaaggatattcagagcggctttatttataattcatatacgtatatgtgtatttagctttagtaaatattgccaagtaattttgcaaagtgctcgtaacactgctttaggttttgtcacggaagaggaagttggaggccccagtaggagacaggaataatcagtagcaggggtgataatgggccattgcaatccaaaggcatgttgtctatgaccaggaatggcaaattgctgatccttgcaatatgtaatgagtggaaaatgctgagactttgccacctggcccattcttaaacatctccttagagagctctgcccggagaagagctacttcacgcgacgacaaccgccgtttccgacctcacaggtccaggcgttcccgacgctctcgctccgacaacgccctccacctggccagcgaac

SEQ ID NO: 296 - DNA, 368 bp, Homo sapiens
ccgaccccccggcgcggccgtgcttctgcccctacaaggtttgggccgaggtgggggagggtcctggttgccggccccgccgtcacctccccgccttttaggcaccgcgtggccgggacgtcccagtcgcctccgtcctcctcgcctgccaccggtgcacccagtccgctcacccagcccagtccgtccggtcctcaccgcctgccggccggcccaccccccaccgcagccatggacgccatcaagaagaagatgcagatgctgaagctggacaaggagaacgccatcgaccgcgccgagcaggcgaagccgacaagaagcaagctgaggaccgctgcaacagctggaggaggagcagcaggccctcc

SEQ ID NO: 297 - DNA, 541 bp, Homo sapiens
ttttgagaggctagtaacatcagttttattgggttggggtggcaacatagcctggctgggggtggggctggcctcacaggttgttgagttccagcagggtctggtccaaggtctggtgaatctcgacgttctcctccttggcactggcaaggtctcttctaggtcatcgatggttttctccaactttgccacagacctctcggcaaactctgctcgggtctcagcctccttcagcttctcctccaacagtttgatctcctcttcatatttatcttctttggtggaatacttgtccgcctgggcctccagggatttcaagttgttggtaacaattttcagctcctcctctaggtccccacatttactcctcctctgaggccatcagggacttgagggcctggtccatggttcgaagttcctcctccagctgtctggctcggctctcggccacctcagccctctcctccgagcgctccagctctccttccaggatcaccagcttcctggccacctcttcatatttgcggtctgaatcctcagc

SEQ ID NO: 298 - DNA, 397 bp, Homo sapiens
  misc_feature (11)..(11): a, t, g, c
aattaatatannnntgaaacaatttggtaatgcagactgggacaatgtcactatacccactgaggtaactgagacatgagagctttccaacccaaggtcagaaatcactgaaacagcttatgaaaccctcactattttagtgttttaaattcagaatgctgtatttttctcttgggcaacagataaccttcatttttgacgtaaatgataatggtggcttcacatggcccagggcaaacttcaaaacaaaactgtagggaccacatttttctaacctggaaaataagtcatataaggggtccaaataagggtaaaatttgggaatttaattattcctggggggaaaatattgtgggggtatccctggtatcctngggcccggtaatcccgggtttta

SEQ ID NO: 299 - DNA, 352 bp, Homo sapiens
  misc_feature (326)..(326): a, t, g, c
tcaatttaaaaaattacacatggaacaagccaaataaatatttttaaaatactgtttttatttagagagagagacagctgtataaactgctctgtgtatgtgtaaaacattgataatacattagacataatttgcagaaactactaattttcacatcatttccaagtagaactgcaaaggtacagtactggtttagctttatggttaaaagcattgggctctaggagccaggataacacaggcttcaaaccctagttctgccgtttactgggttgtgttaccttggggtagggttacttggacctttttgaggcttccaggtttcntaatcttttattttaatggngttttt

SEQ ID NO: 300 - DNA, 169 bp, Homo sapiens
aaatttaaaaagatgtcctcactgcacaagtgactacgggctacaggcaaggatgggagacggaggcttcaacacaactcattgcacttagaaccgttactaaccgaaacaccatttgcttgtcaacaatgtacccttgacagcagggagaaacttctttatagtctct

SEQ ID NO: 301 - DNA, 219 bp, Homo sapiens
  misc_feature (203)..(203): a, t, g, c
atcacagagaaagactcagaccctgcagatggagaaggcccagagacattaagctcagcactctctaaaggagcaacagtttacagcccttccagatacagctaccagctcctgcagtgtgatagtcctcggacagaatcacaaagcctccttcagcagagttcctcccccttcagaggacattcctacacagtctcccaggntacanttatcggaact

SEQ ID NO: 302 - DNA, 469 bp, Homo sapiens
  misc_feature (379)..(379): a, t, g, c
actgtgataaaacatacacagtttacaagaatatgaaatgctttactaattcgtgtgtcatccatgctaatcatctctctattgttccaattttagtacaggtgcttttgaagtgggcactcttaattttttgaacattttctaggtttctgataccatactcattctgtgtcttacctatcacaaccccagaatcagccatttctccaaattccttttagtggagagtggtatttagaaaccaggatctggacaccatttctctttttgttattgttgtttgccttgctttaatgatagctctttttattaatttttccattattataaaagatggccaaatacatacatttctatggaaaatgaatcaagtcttatntattttacagttaaaatttcattattcctattttaactgataaaccagtttaattttcaagatgtattaaagtctcccacaattgtattc

SEQ ID NO: 303 - DNA, 299 bp, Homo sapiens
  misc_feature (296)..(296): a, t, g, c
aatagaatacaattgtgggagactttaatacatcttgaaaattaaactggtttatcagttaaataggaataatgaattttaactgtaaaataaataagacttgattcattttcatagaaatgtatgtatttggccatcttttataataatggaaaaattaataaaaagagctatcattaaagcaaggcaaacaacaataacaaaaagagaaatggtgtccagatcctggtttctaaataccactctccactaaaaggaatttggagaaatggctgattctggggttgtgataggtnagc

SEQ ID NO: 304 - DNA, 606 bp, Homo sapiens
cagttctctagtatctttattatccattccacgggggcactcatggctgcacaactcaggggtgcagatgtctccctgcaccatgccaggtgtggtgcagaatgggaggcagggggatggagcagagagaggcctggactgcctgtggctcttgtcgagtcactgcccttctgtaggctctgctctggaaatgctggggtcagaggcaggtgttggggagcagccctgtctccctctgtcctccaggggagcagctgggcctgaggaggaagcacccctgccccagggtcccgaagccccagaggacccatatccccctggggcagttggttgcccctccctgaccttgggctctggaaggtcagagcagcagagcagactgcccgttttcccccatctcagggcctggctgaggcagggcaggcaggagagaggcccctgaggccgggtttcaggccctgggccaggcactcagatggggtgcacgaactggtagacgaggcgctgggaaatgtctggcttccggatgatgcccttcttgtaatactggccgatggagccgctcagcttgtcgtagttcatggcgggacgttcttgcggatcccc

SEQ ID NO: 305 - DNA, 514 bp, Homo sapiens
ggagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggaccagggcatgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacag

SEQ ID NO: 306 - DNA, 375 bp, Homo sapiens
  misc_feature (298)..(298): a, t, g, c
tatttgagcaatttattttttttcaaagaaatccatgcaaagagagaacgatagaaagggagggagtgagagagaaagacatggtaaccatgttctagcagccaaacataggagaataatttggcacaacttgatggttttttttctttctttgcaaaaggacaatctatatgctaccactaaaatgtatcttcctccaaaagataccatttgattttcgaaaacataacacatgggttaggtcctctacttcatgggctgggagggagggtatacagggataacaaaaaaaaaaaantagggnaaaantaaggaaagtgggttcccctttatcggggggnggaccccnggggtgctntagggggcaggcacatg

SEQ ID NO: 307 - DNA, 433 bp, Homo sapiens
  misc_feature (243)..(243): a, t, g, c
tggaatgataaaagtgaagaatcagctccgcttccttgcagaactggcctatgatctggatgtggatgatgcgcctggaaacagtcagcaggcaactccgaagacaacgagataagcacctttcacaacctcgggaacgttcattccccgctgaagcttctcaccagcatggccatctcggtggtgtgcttcttcttcctgggtgcactgactgcctgggtgcccagcacatgtgctgccctnacagcaccctgtgggtcttccttcgataaaaggggaaccactttcttatttttttctatttttttttttttgttatcctgtataacctcctccagccatgaagtaggagggcttaaccagtgttnatgtttttcgnaaatccaaatgggtatctttttgggagggaaggtacattttagtgggtaggcna

SEQ ID NO: 308 - DNA, 491 bp, Homo sapiens
  misc_feature (393)..(393): a, t, g, c
ctgaaactgtcggaatatatgggtcttgaaattcagaagatgatagtcactcttcccatatttataggctattaaggcaagggatatcttaaacatcatattactttatttagatttctactactccaattattaatgttatgtatttctcattgttttacttcttcatggtattatgaagactatatagatgattcaaccaagcctgcaaatctccctcttgtgggaattccactgggacccaatctgttttccatttccattgcaatactactaaagccatacaatatcaaggcaccctccctctagggtccaggggactatccacaggaggaggcagggcatgtaaggattttaagggactgggtttcgagggggtccgagtgtaggggnaaacagccntgtttgncatttgttaggngtggatgtccatcttgnaggagccgctgggcctggatgncctgctgttttgactccnggcatacccagan

SEQ ID NO: 309 - DNA, 471 bp, Homo sapiens
  misc_feature (6)..(6): a, t, g, c
ggtttngagataaaccaattttatgtctatcatgttatacaaaaatctagaaataatagatttgtacagaaaaaaatgataataaatgagancacaaaacatatantttaaatttggnatttttccccccatgatattaggntgataatcatttcaaagcacatgtctagcttcagagtaggntttgtncactggccaaagcctgccatgaaactatggctttcagcatctgtctgctctactggctcttgacaaaactcttgaggtnttcaagaaaagtaatgtactcctggggcnccgggctgtgctgagctccaccagctcatctgcaaaagtnttgtccacccctcggtcggcaaggaaatccattaggtggtcatataaggcccagtccaaggaatccgtgttgagtgtataattagtatccccncgttcccgaatttttggcctcgagggcaaattnccccatag

SEQ ID NO: 310 - DNA, 438 bp, Homo sapiens
  misc_feature (1)..(1): a, t, g, c
ngggatactaattatacactcaacacagattccttggactgggccttatatgaccacctaatggatttccttgccgaccgaggggtggacaacacttttgcagatgagctggtggagctcagcacagccctggagcaccaggagtacattacttttcttgaagacctcaagagttttgtcaagagccagtagagcagacagatgctgaaagccatagtttcatggcaggctttggccagtgaacaaatcctactctgaagctagacatgtgctttgaaatgattatcatcctaatatcatgggggaaaaaataccaaatttaaattatatgttttgtgttctcatttattatcatttttttctgtacaaatnctattatttctnagatttttgtatnaacatggatagncataaatttgggtttatcctccnccaaaa

SEQ ID NO: 311 - DNA, 255 bp, Homo sapiens
ttaaaaaatcaactacagagaaagggccatgtcttcgagtccatgttcacattgtcccacgctgtcgcaccaggatgggccgcatactttgtcctggctgggactgtggcctcttggaagggtactgggccactccagggctcgggtgctgctcctcctgggatccactagagggtgaactctgggccctcccagactcctgctcctctggggcccccagggactctgggcaacccttccccttgctccaaacca

SEQ ID NO: 312 - DNA, 305 bp, Homo sapiens
  misc_feature (224)..(224): a, t, g, c
gataagttccctaacatctccagctcctggctctggtttggagcaaggggaagggttgccagagtcctgggggccccagaggagcaggagtctgggagggcccagagttcaccctctagtggatccaggaggagcagcacccgagccctggagtggcccagtacccttccaagaggccacagtcccagccaggacaaagtatgcggcccatcctggtgcgacangtggggacaatgtgaacatggactcgaagacatggccctttctctgtagttgattttttaaatgtgccattattgttttta

SEQ ID NO: 313 - DNA, 449 bp, Homo sapiens
  misc_feature (2)..(2): a, t, g, c
annagtggatattncacagtttatttccaaacactcagagganagggggtggcctatgggtccatctgcctcccctccctgcatttggctgaatcaagaacttctcccccctgacccccaagaccctaggctgtgccaatagagaaggcactgtcaccccccaccctgaggagacctagacaggtgaggagcatggagggtgggggaacagagttcaagtattcacagttcccccatctacaccccgggattacactgtgccagagccatgagggaggatcccacctctgggattcctagtgggtgttgatagtccttctcccacttaggaaccctccagatgaactccctactccttttccctggaaataacaaaccaaggtcctaaggccctgtagtttggggcaagggagggagtntaggnagggccattnacccaagggcccntt

SEQ ID NO: 314 - DNA, 409 bp, Homo sapiens
  misc_feature (227)..(227): a, t, g, c
ctatgttccccaggattcagctattctggaagatcagcaccctaagagatgggactaggacctgagcctggtcctggccgtccctaagcatgtgtcccaggagcaggacctactaggagaggggggccaaggtcctgctcaactctacccctgctcccattcctccctccggccatactgcctttgcagttggactctcagggattctgggcttggggtgtggggtngggtngagtcgcagacagagctgtctgaactcacgtgtcagaagcctccaagcctncctcccaaggtcctctcagttctctcccttcctctctccttatagacacttgctcccaacccattncactacaggtnaaggcttttcacccccatcctggggnccttnggttgagttgcctggtta

SEQ ID NO: 315 - DNA, 308 bp, Homo sapiens
  misc_feature (16)..(16): a, t, g, c
cacttcaattttaatncttgaagaacagcctcgacaacatatatcgcactcattataagaagcaaagggttatatcaaaaattattagtatagaatcacaggaaatctggtttggaaccagaaaaaaatacaaaacagatatagatacatagacacaggacaattttttataattatttggaaaatgttatttnccccnaattcttgtttcctttttctttatgcgcatacgatgtttaaatttnacaaaaaatgaaaataaagactagtattntacaaaatgaataacaatgttcagtgtgtgtgtg

SEQ ID NO: 316 - DNA, 388 bp, Homo sapiens
  misc_feature (357)..(357): a, t, g, c
aagcatgggaaaaagagactcttttaggatcagatctgtgagcacgttggcgaggaaaaacaaaacaaacaaaaaaaagaaccttgtgtctgtctggtgaaaaaaagaaaaacaaattggaagagaggaccatgagaattttaataaaacagaaggaaactaatggaccttccaggatttattgtggacggatgtggatatattctgtacaggaacaacacatatggaagtggactgaagcctatgtagaaacacacacacactgaacattgttattccttttgtaaaatactagtctttattttccatttttttgtaaaattttaaacatcgtatggcgccttaaagaaaaagggnaaccagganttagggggnaaattacnttttt

SEQ ID NO: 317 - DNA, 514 bp, Homo sapiens
  misc_feature (374)..(374): a, t, g, c
gaatagctatggaagttctctttattgattacttaatgtgtaacaataattggcatctttttcacacattacaaaaaattatacttggctcagtatgcaaccttttaagcatagccatattatttaacaaaagaggggaaaacctattctacccaacacagcatttacaaatgcacaaaacatgccactttggcttgtatattgtctagattaaaaaaaatcttttaacataaataagttagtataaatttttcagtgtttttacagagttatgtacacaggtacacttcaaatggtttttccatacacaggcaatgaaatactggtttaaagatgtagtatccatttcacttatcctacaagtgtgcttttcnctacatgaaccttccctgaaaaataacactttccngtgggataaatggacngagtaagccttaaattaaaaaatatttcnatttttaatgggaagttacctacagatccttnaaaatatcggggggnttaggaattaatt

SEQ ID NO: 318 - DNA, 505 bp, Homo sapiens
  misc_feature (4)..(4): a, t, g, c
tccnnttcagagacagctgatacttcatttaaaaaaatcacaaaaatttgaacactggctaaagataattgctatttatttttacaagaagtttattctcatttgggagatctggtgatctcccaagctatctaaagtttgttagatagctgcatgtggcttttttaaaaaagcaacagaaacctatcctcactgccctccccagtctctcttaaagttggaatttaccagttaattactcagcagaatggtgatcactccaggtagtttggggcaaaaatccgaggtgcttgggagttttgaatgttaagaattgaccatctgcttttattaaatttgttgacaaaattttctcattttcttttcacttcgggctgtgtaaacacagtcaaaaataaattctaaatccctcgatatttttaaaagatctgtaagtaacttcacattaaaaaaatgaaatatttttttaatttaaagccttactctggcccatttatcccacagg

SEQ ID NO: 319 - DNA, 249 bp, Homo sapiens
  misc_feature (244)..(244): a, t, g, c
ataataaggaaacgtgttcacttattgactattatagaatggaactcatggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacagggtgaccaataacccngacgc

SEQ ID NO: 320 - DNA, 376 bp, Homo sapiens
  misc_feature (172)..(172): a, t, g, c
tttttttttttttttttttaaagggtatgtgaacggaaacacatttattacaaaaaaaaaaaaccccaaaacgaaaaacaaattcacattgtattgagctacaatatggcagcagattaaaaaaaaatatttttacacagtttaaggtaactcctaacagaacatagccttnttgccacgagacaggacacaggnttccaagtactcagtagacggcgagtgaagcggncatcgctggggcctgctcccccggctctggcctccaaacccgccctgcatgccaccgtntggnatggggtggccccgggaggccccgcctggggaaaaagttgccgcgccctgacattcctcctcggttcatcatntgggccagggc

SEQ ID NO: 321 - DNA, 466 bp, Homo sapiens
  misc_feature (321)..(321): a, t, g, c
atgaagggctccatttgatacttccttcactgtggaaggaagaaatttgtaaaaattatttccagaatatgcacacattaaaggtttataaacctgacctttctttagtagaagacagtaagagaatactgttggctctgggaactaaccatttgagctgtgggacccagggaatctctcttgtaagcatcatctttagtgggtgactcctggtggccctcccagctctggtggtgctgtggggacctgccttggagatgccttggtactagtaccttttttgtaccaggccatgagcctagggcacagggcttctgctgncagatttcaggaaggcagctgtgttgtgtgaaccgatacagcctgccagcacttctgtcccgagttgactgtcgttgttcatccttagcatgtttgcctgaaaaagcntttggggtcttgggcgttgacattgaagttttttttt

SEQ ID NO: 322 - DNA, 341 bp, Homo sapiens
ccattttggggtggagaaggtggatgtgatgaagccaataattcaggacttattccttcttgtgttgtgttttttttttgcccttgcaccagagtatgaaatagcttccaggagctccagctataagcttggaagtgtctgtgtgattgtaatcacatggtgacaacactcagaatctaaattggacttctgttgtattctcaccactcaatttgttttttagcagtttaatgggtacattttagagtcttccattttgttggaattagatcctccccttcaaatgctgtaattaacaacacttaaaaaacttgaataaaatattgaaacccctccgtgcc

SEQ ID NO: 323 - DNA, 350 bp, Homo sapiens
ggtttcaatattttattcaagttttttaagtgttgttaattacagcatttgaaggggaggatctaattccaacaaaatggaagactctaaaatgtacccattaaactgctaaaaaacaaattgagtggtgagaatacaacagaagtccaatttagattctgagtgttgtcaccatgtgattacaatcacacagacacttccaagcttatagctggagctcctggaagctatttcatactctggtgcaagggcaaaaaaaaaacacaacacaagaaggaataagtcctgaattattggcttcatcacatccaccttctccaccccaaaatggcacaaaaaaaacaaaaaag

SEQ ID NO: 324 - DNA, 479 bp, Homo sapiens
gtgttagagattatttattattactcttaggggaatgaattctggacaagggatgtgaaaattccagaaaccctcgattgtgaccaaaatgactgtaaaatatggagcacagcctgtcactatatgaatataaatggcctttgcagagaggtaagttctttggaaagaatggtctccagctatatgtctcaagcttcatggagaggggcagggatgaatggcagggaggaagcaggaggtctcaccagcagaatccaggagctcactggggacccatcccaattcttaaagcatgggtaattcagccaggcctcagcctcttgtgccagctgcctccaaccctttgggtctccaccacccaagtttcctgtagggtccgccgggtccaggatcacaggcctgggtttcgtgagctgccttctcaggtacttttcaataatggggtttttaaagtcataatactttgtccagtagatgca

SEQ ID NO: 325 - DNA, 554 bp, Homo sapiens
gagcgagggagcatgaaaacacatttcaacacagactgtggatttcggacggtcttggcattagtcataaactaccagcaactctgcatctactggacaaagtattatgactttaaaaaccccattattgaaaagtacctgagaaggcagctcacgaaacccaggcctgtgatcctggacccggcggaccctacaggaaacttgggtggtggagacccaaaggttcggaggcagctggcacaagaggctgaggcctggctgaattacccatgctttaagaattgggatgggtcccagtgagctcctggattctgctggtgagacctcctgcttcctccctgccattcatccctgcccctctccatgaagcttgagacatatagctggagaccattctttccaaagaacttaccccttcccaaggccatttaaatccctaaagtcacaggctgcgctcaaaatttacagtcattttggccacaatcgagggtctcgggaattttcacatcccttgtccagatttcattcccctaagagtataataataatctcta

SEQ ID NO: 326 - DNA, 331 bp, Homo sapiens
atttacaagtccttttattttgcatgttcattgtaaatttaatactgtaaatgtattcaaattcatttacatgcctatggttgcctttgattaaacttcttccaaaaaataaattctgcccagatgttgttgactttattatttcatgtcagattacttgaatatacgttgctgcataatcactaccaagtgggttctttgctgtgcatttgtatatcccagaatcggaggtttggggattctgaatgactagggtaccttgtaagtgaagctgctcacttccatgtgtcctctctttacttgccgttgagagaagggagtggtcaggcat

SEQ ID NO: 327 - DNA, 446 bp, Homo sapiens
tccagatttctgtgatcccactagccccatgttgctcaacacaaatactattaacttgtatactatacctctttggcccttgctggactgtaaaaacctgtgactaaactatttcctcttatccatttcagtgataaattctgcagtacttgatactgtttagccattgctaatttgactaattaagtggactgtgacaccttttgaatccctatagattatttatataaaaactaactggggatctttacttcgaatggagaggaggaagaatgaaaaaaattgtgttccattactgttttaaaaaattagagtacaccatctttggtctatatcaatcaaaggaaaacaagaatagtgaagaatatgaatttttagagtttgtcctaactcatgcttgtcctattaaaaagaaagtaacaaagtgttactataagtttatatgc

SEQ ID NO: 328 - DNA, 457 bp, Homo sapiens
aattcggaacgaggctatttagaaataagctaattggatcagtggcttaaataatagctgactgtgtgtacatatgtatataatatgtatatacaatatcaggcatgcatgtggcttggaattttgtttcctccataaaatgtggaagtgaattaaacaagttttagtcatttatacaaagtcacaaatataaagttcagtttgtcacaagattaaattgctcacaaggtaaaattgtattgtttggcaaaatcacaagtaacaatcctgtgagttttctattatgaaggttaataataaatgggctcatttagttgcctgggcacctattcacaaattcatttgtcagcctctttttagttctcttaaaaaaaaaaaaatcatatgatcattttcctttttgggggtacttagcttccatgcctataaagtctggtaccagactgacttgaaattc

SEQ ID NO: 329 - DNA, 494 bp, Homo sapiens
tttcctatgctgggaggaagcattttaatcagatggtcggggatggtaacactgtacagggagaactcacagctccaagagcttccctccaaattatgatgaagttcctgtaaataactagatttcctacagcctggtcctctgcccatgggaaggaggggaaatggatgtgaaaagcaatgtggtctgccctcctgtcattcatagatccagttctgagcacagctcgtgtaatctaccagttcctcagggtgaaacacaagccgatctccttctctgcactctccacagaatcactgccatgtataatgttcctgccaacttgtatgcagaagtctccacggatggtcccaggcttggagtctgcagggttggtctccccgagcatgactcggcccgtcttcaccacattcagcgcctccagaccatggcaataccgggcctgagtgcatgtatttcacaggccggcaagaatggacggtccttcagtcaac

SEQ ID NO: 330 - DNA, 507 bp, Homo sapiens
aattcggcacgcagggttgacctgaaggaccgtccattctttgccggcctggtgaaatacatgcactcagggccggtagttgccatggtctgggaggggctgaatgtggtgaagacgggccgagtcatgctcggggagaccaaccctgcagactccaagcctgggaccatccgtggagacttctgcatacaagttggcaggaacattatacatggcagtgattctgtggagagtgcagagaaggagatcggcttgtggtttcaccctgaggaactggtagattacacgagctgtgctcagaactggatctatgaatgacaggagggcagaccacattgcttttcacatccatttcccctccttcccatgggcagaggaccaggctgtaggaaatctagttatttacaggaacttcatcataatttggagggaagctcttggagctgtgagttctccctgtacagtgttaccatccccgaccatctgattaaaatgcttcctcccagc

SEQ ID NO: 331 - DNA, 483 bp, Homo sapiens
cacagtctgggggccccgaagcccctcaatctttattccagttcctcagatgtggcagttcccccagcttccctggaaacaagtatcagacaaaggtggtggcagacacaggacagggccctgggtggggcttgctggcgctaaaaggatttctcagcaggctcagggtctcccgccccgaatgacccttctggccacttcaagctactcctctgctgcccggcttccctctgcctctggttcccctggctgagcagggccttcctgggcttgaccctggcccccttcagcagttgtctcagactgttcccgaatgtccctggggctggctttgaccccagcatccccatctccctggcacccttgctcctcccctctgcttcttgcctctgactgcccactgccctcctcctctgtcacagccccctgggtctcaggccactctgcccggcggtagacgaggtagccagtggtgggcagctg

SEQ ID NO: 332 - DNA, 492 bp, Homo sapiens
gcccacaccctcccactgagccccgtgcaggagctacggcgctccctcagcctctgggagcagcgccgcctgcctgccacccactgcttccagcacctcctccgagtagcctatcaggatcccagcagtggctgcacctccaagaccctggccgtgccccagaggcctcgattgccaccctgaaccagctctgtgccaccaagttccgagtgacccagcccaacacttttggcctcttcctgtacaaggagcagggctaccaccgcctgccccctggcgcctggccacaggctgcccaccactggctacctcgtctaccgccgggcagagtggcctgagacccagggggctgtgacagaggaggagggcagtgggcagtcagaggcaagaagcagaggggaggagcaagggtgccagggagatggggatgctggggtcaaagccagccccagggacattcgggaacagtctgagacaactgctgaagggggc

SEQ ID NO: 333 - DNA, 440 bp, Homo sapiens
tttaagtttatttttaacaacaaaaaaatctgtatttttctcctaaaaggtaccaaaatttgggcaactataaatgtttcattctatacttagaaataaaataaatggtttcttttttatcattatctgaaagaatggattgtctggcttcttgaaatccatggcctcttactgggacaggaaaggcattggagttatgtccctagactagaaagagtatggttcatgaggtccctggccctctccataactctgcatttggtcactaaatattggtagaaaaagtatttgtccattactttacttgttccaccacctagagccaaaggagttagaaggtaattaatagtgatacggatagaatgcaagccacagtcacctccatccttctcaccatctctcaagcccatgtccttgacctttggtatttacatccatgt

SEQ ID NO: 334 - DNA, 321 bp, Homo sapiens
gatgaataactttgacaggctggaaaaaggtgacatttcaggtagagcgtgtcacatggatgtaaataccaaaggtcaaggacatgggcttgagagatggtgagaaggatggaggtgactgtggcttgcattctatccgtatcactattaattaccttctaatgcctttggtctaggtggtggaacaagtaaagtaatggacaaatactttttctaccaatatttagtgaccaaatgcagagttatggagagggccagggacctcatgaaccatactctttctagtctagggacataactccaatgcctttcctgtcccag

SEQ ID NO: 335 - DNA, 241 bp, Homo sapiens
tttaagaaaagtaaattcatcttgctcacagtcctttctggaagagtttagaaagcaaagaattcaccgactcagcaggaagcagaacgagctgttccttcttttgacacgcacaagctaatcccctagagagtggggatgtgggaaacggagggtaattaattctttggtcactggttcaatgctgaatagccttggtcagttttggctctctcctattttagggggaaaaatatttttg

SEQ ID NO: 336 - DNA, 564 bp, Homo sapiens
gcaggtcagcaacaagtttattttgcagctagcaaggtaacttggtagggcatggttacatgttcaggtcaacttcctttgtcgtggttgattggtttgtctttattggttgggggtaggggaaagcgacgagaagtaacatggagtgggtgctgcctccctgtagaacctggttacgagagcttggggcagttcacctggtctgtgaccgtcattttcttgacatcaatgttattagaagtcaggatattttttagagagtccactgtttcttgagggagattagggtttcttgccaagatccaagcaaaatccacgtgaaaaagttggatgatgcaggtacaggaatacacgatggcatagttctcatagtcggtggccaggttccagtacggtgccgatggcataaaccaggaaaacttaacttccagcttggcaggctctgtgaggttaactggggtggcttcaccttcgatttgattcacagttccatcagctctcaactcctggtttaacactttgatctttccgttttccatagtgagtagttggcccgtatgcagc

SEQ ID NO: 337 - DNA, 490 bp, Homo sapiens
ctgattctgcatctggaaactgccttcatcttgaaagaaaagctccaggtcccttctccagccacccagccccaagatggtgatgctgctgctgctgctttccgcactggctggcctcttcggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccaggccaactactcactaatggaaaacggaaagatccaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtaactgcatcatc

SEQ ID NO: 338 - DNA, 394 bp, Homo sapiens
  misc_feature (1)..(1): a, t, g, c
naanaanatgttgaccatgtagttttatttcatcagtactcccatttttaatggattcaggcagcaccccagagtacaggactgagttcctaggggtggcctgacccagcagctgtcttctgttccaggnggaaaaagctttttattaaaaaacagaaacgaaactttagaaaatatgaaaaatcagagggaggacagaggtcatttcataaaatcgttatttgaaaacagtgcagtgtgcaaataacactcagttggggaatcgggagaccgggggactagtctaactgggctcaaactaggctgtgaccttagggcgtgtcattcaccacttgggggatcctttcctcatctaaagcattcagcacagttaccctgttagggcacttttacg

SEQ ID NO: 339 - DNA, 413 bp, Homo sapiens
  misc_feature (33)..(33): a, t, g, c
atgaccctttgcctgttccgcatcctggaggcngcaaagggtgaaatccgcattgatggcctcaatgtggcagacatcggcctccatgacctgcgctctctctgaccatcatcccgcagaccccatcctgttctcggggaccctgcgcatgaacctggaccccttcggcagctactcagaggaggacatttggtgggctttggagctgtcccacctgcacacgtttgtgagctcccagccggcagcttggnacttccagtgctcagagggcggggagaatctcagcgtgggccagaggagcttcgtgtgcctngcccgagccctgctccgcaagagccgcatcctnggttttagacgaggccacagctgccattcgacctgggagattgacaacctcatncagggttaccatc

SEQ ID NO: 340 - DNA, 396 bp, Homo sapiens
tataattattttaacatttttattgaatgatttcaacaccttggtttatgggttgttttgattttgtttttttgagacagggtctcactctgtcttgaggggggaaaaaaaccatgcaacaatggaatacatatatttaaataactgcacataatacaccctccatttttgttaccaagagaaagcttccatcaggtagctttaccaatacatttccattcccatgaaatgcaataccacagtactaatattctattcagaaaaagactacaattcccttgtaactctttttctttttcccttgtaactctgagttctttcttttaatcgttcaggccacataggtaaggccattttagaaaaggactgctttcaagtatctaaatatacttccgg

SEQ ID NO: 341 - DNA, 299 bp, Homo sapiens
tgagttttcatctgattgtggggaccaagtcctgagtagagggccaagagctagggacagggggaagagactggcccaggtggtagggaggaaagaactcccagagtttcctttagccaggaaacctgctctactgaccccgtgacttggacagtcaagcatcaccctgagagtgacaagtgtaaaatgactcccttcctcccccgccctccggaagtatatttagatacttgaaagcagtccttttctaaaatggccttacctatgtggcctgaacgattaaaagaaagaactcagag

SEQ ID NO: 342 - DNA, 430 bp, Homo sapiens
cctgtgttatcgatttattgcagctccaatatgagtccactcctactcaaaccctgattctcaggggcctggggaaggcaccccactagggccctgtccccaccagagcctggacatgggactctctcccaagcaggggtgttgtccttcacaggggcttccacgtctctcccgggcctgtccgcactctgggcagggctggcacctggctattccctcaatctgggccctgggcactggcaatgtggaatggtccaggtgttcatgtcagtggggcttggggacatggccatccacgtagaagttcctggtgatcttgggcagggtgaattccgctccttccagctccggtgtcagcacaatctggcagcccagccgcgagttcctcctggaggaaggggggcatgtttagcaagtcgtcttcctcttc

SEQ ID NO: 343 - DNA, 447 bp, Homo sapiens
gagcggcctgtgaagcctccctggcctgctccacctgccatgtgtatgtgagtgaagaccacctggatctcctgcctcctcccgaggagagggaagacgacatgctagacatggcccccctcctccaggagaactcgcggctgggctgccagattgtgctgacaccggagctggaaggagcggaattcaccctgcccaagatcaccaggaacttctacgtggatggccatgtccccaagccccactgacatgaacacctggaccattccacattgccatggcccaggccagattgagggaatagccaggtgccagccctgcccagagtgcggacaggcccgggagagacgtggaagcccctgtgaaggacaacacccctgcttgggagagagtccatgtccaggctctggtggggacagggcccctagtggggtggccttccccagg

SEQ ID NO: 344 - DNA, 284 bp, Homo sapiens
tttttttttcatgctttgtttcactttattggatacaccgggtgtgggatttacaaataggaccaacaatgtgtgcggggccagacggctgcctgtgggtctgtgtgtaacaggggctgtaaaatgaatggacctcatgtgcacgtggtacagagagaagcaaaccaagaccaactggatcccaacgtgggtaacataaacagttaaattactaaagtcatcttaagaccaaaatacttcaatgaaaatggcatattaaaaacatataaattacatttaagctc

SEQ ID NO: 345 - DNA, 319 bp, Homo sapiens
tttttttgcagccaaatacagtttatttttttctctctcagctgaggagcagacacatttacggagatgcaaatgaccatccagtggcctacagtggagggggggtggagggagtaggggattagggggttggtagggagtgttgggggagggaggagcacagggctggctcagggccaggatctgtgaacggggcagggctctgtcaacatttacccagtccatccccagagagggctgcaaaaggggacggcgatagaaaagtagatggaatactttgggatgggggacaacttacatgcaagaaaaggagggaact

SEQ ID NO: 346 - DNA, 355 bp, Homo sapiens
  misc_feature (1)..(1): a, t, g, c
ntctttattcatacttgttgacatttcccatggggaatggttgaagggaggggcagggagggctgagcgggaagccacggcttggttttcctccatccagtggagaagagactcggtagggacatgctaagtagagtgaacaggggaaccatcactatgtgggctgagatgcactgtgcagtgacttcagacaacctgagtccttctgtcaggaaatgcaatttctaagtggaaaaaaaaaacaagacccaaagaaaaaaacaaagtgcntacaaagcaaactgctaatgtaagaaaggttctaagggcagccagcagttgaaaagccngggggtctgggagtgggccacctggg

SEQ ID NO: 347 - DNA, 485 bp, Homo sapiens
ggctgactgtactcctcccaggcgccccttccccctccaatcccaccaaccctcagagccacccctaaagagatcctttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggcacttccagacccctgggcttttcactgctggctgccttagaacctttcttacattagcagtttgctttgtatgcactttgtttttttctttggggtcttgttttttttttccacttagaaatttgcatttcctgacagaaggattcaggttgttctgaagtcaattgcaacagtgcattttcaagcccacattagtgatgggttcccctgtttcaattttaattttt

SEQ ID NO: 348 - DNA, 315 bp, Homo sapiens
  misc_feature (4)..(4): a, t, g, c
ttanataaacagctttatttgccttgtacagcatcaattttcttacattctcagttaattggccattaaagtgctggaaattttcttaatcatgataacatttgttaaaaagaaatcagaactaatatcaggaacatggcggcatgaaggaaacagttcccttacaaaacacagaaaatggaagcccctcatgttgaggggggtggggttggacaatttgcaaacagattctaatttcctctcaccgtcagcaccaaactggctgggnccaccacccctggggngaaagggaaccancactaagggacccctaaa

SEQ ID NO: 349 - DNA, 372 bp, Homo sapiens
  misc_feature (283)..(283): a, t, g, c
agcaaacgcccacagatggcccagaggtggtggtagtcagggtgtgtgggtgtttttagggttctttagtgttgtttctttcacccaggggtggtggtcccagccagtttggtgctgacggtgagaggaaattagaatctgtttgcaaattgtccaacccaccccctcaacatgaggggcttccattttctgtgttttgtaagggaactgtttccttcatgccgccatgttcctgatattagttctgatttctttttaacaatgttatcatgattaaggaaantttccagcantttaatgggccaattaactgaggatgtaagaaaattggatgctgtacaaggcaattaaagctgtttatttaacctttaa

SEQ ID NO: 350 - DNA, 237 bp, Homo sapiens
  misc_feature (156)..(156): a, t, g, c
tttttttttacaattttatttataacaatatctgtgttatttagttgtaaaggaattcagcaaaaattattaaaacgttcacgtccccaaaacggggctgcccgctttcccttcctgggtgtggcccacctccttggcgtccccaggggaggggangctggangaatgtttaccagaggaagtctccanccctggggaacctcggtnggggccctgggagacagggagggcctttct

SEQ ID NO: 351 - DNA, 370 bp, Homo sapiens
  misc_feature (95)..(95): a, t, g, c
ctccctgcctgctccttggcacccccatgctgccttcagggagacaggcagggagggcttggggctgcacctcctaccctcccaccagaacgtancccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtgggctctgggtgaggtagggcgggaaaggatgggagtgttttagttcttggggggaggccaccccaaaccccagccccaantccaggggggcacctatggagatgggccatggttg

SEQ ID NO: 352 - DNA, 385 bp, Homo sapiens
  misc_feature (368)..(368): a, t, g, c
ttttttttttagattttagacactctcctgccttattctagggtcaagtgggcccttaacaggaggtcttgatttttcaaaaacatgattcggaacattactcctcttcattgtcatctgcttgcttcacccaaagttcttggtggctcttgctaagccagaacatgaaccatccattgtgtctcctccagccttctctccatagccgtgggaggcctgtctggaaacatgtgagtcattcgtgctgtcccttagggcagttcaaactctacggttcgtgggaacgagggccctctgtggctgatgggggcctctgctgcttgggtttccttgtggctcctttgcccggttgaaggtcttccaacctncctcttcctccgtggaa

SEQ ID NO: 353 - DNA, 488 bp, Homo sapiens
  misc_feature (391)..(391): a, t, g, c
atcgagacagcaccatgaatgtgtcccagaacctctacgagctcctccccaggacttcgccactgaagaataagcactttgggacttgtgccatcgtgggcaactcgggggtcttgctgaacagcggctgtgggcaggagattgacgcccacagcttcgtcatcaggtgcaacctggtccccagtacaggagtatgcccgggatgtggggctcaagacagacctggtaaccatgaacccctcggtcatccagcgggcctttgaggacttggtcaatgccacgtggcggggagaagcttgcttgcaacggcttgcacagcttcaatgggcagcatcctgtgggatccctgcctttcatgggcccggggcgggcaagggagcgtgtttgagtngggttcaacgagtttattccttgaaaggcaccacgttcaaacttgcggcatttgcattaacccctttcgttggcggcttgtttgnaacgncttttcg

SEQ ID NO: 354 - DNA, 528 bp, Homo sapiens
aggctcgtttatttattcattgatcaactggcacttcttgaaagcctgctgtgtgccaagcctttccccaaaggaggatatcagtggtggagccaagtctcagggtggaaaggacctggaccacacagagcaggactccagagcctcctccatatggcaggaatcaagctttcacaggggaaacgcaggatttcccacacatgcccatgcaacacttcaagtcacgcttgcactggccatccatctcacagaaattgggggggttaagcatcaaacattggccataagtcactgggcacttcccaggcttcctccttgttgggtttggggtgtcaacaggatccaggcatttgatgccacaagtgtcaggacaacatctcttcttccctggacactgccagtcactctggcactcaggtttcttgtatctaaggcactgggcagatttcttaggaggacagactccagctttgaaggactttccagagccttccacagcccaaggtgccagagttcccagggcaagca

SEQ ID NO: 355 - DNA, 440 bp, Homo sapiens
ttcagttttaaatatttatttaaaatccagaggggaaaaggagaaacggaacccattggggttttaatacactgacatgtggacagagacgtaaacgaagacagcaggaaaacccaagaatgagacagaggccagtggattctggcagcaggagggatccgagcgctgagatgaggcccgagctgctacaaacacgcacttccacgcagagcgtccaggctggggcggcagggcgaggatacagaagtgttgggaggggggacgggccaaagtgaggtattaaataataaaaatcaaatccaattcccaaagagacacaactttaggagagaatacacaaatagagactttcacatacattttccccttctatagagatagttccatggttaaaataactcaaaatccaattcaagcgccgacttgttcgctgatgtagc

SEQ ID NO: 356 - DNA, 415 bp, Homo sapiens
gaaggcagagaggaaggtttaatgagccctgtccaggcgccttcagtggggagcctccttcttcttgcccttctccttcttgcccttctccttcttcttcactttgggcttcttggccttgcccgggatgctctcgtgctgcttggagccagcagcgtgggacttcacatccgtgtccgaatcgctggagctgctgctggagtcggaagagctgtggtgtccttgctggatggaggtgcggcagtgaggcggcgtcccttacccagccccctgaagttggaggcctttggacccggacctgcccctgcctccgaccggccctgaactttgtggggactgagcttgggatctccccgtggcacgccccacaccgggcttctgggaggtgggctcagggctgctccaggtcgaactc

SEQ ID NO: 357 - DNA, 394 bp, Homo sapiens
  misc_feature (22)..(22): a, t, g, c
ttttttgttcatttatattttntttaagagctgtgcccagttttatcatctcacaagaatgaagcaagggacaaaggtaagtgccacgctccctggccactgggttcctggcaagctcccagccactaggtgccaatctcccttcaatgtactccttcttccccagagtgcagaagcgtatgaagacagttatgacatggacacatgcatgagctattatacataattacaaaagctgattctgtcatcaccacatcttgtctcatcagtaggagcgaatggctggcgggacggtggcacagtcagccttgttcaaagttttgtcgatcacgggncctatattccagagtgacctttcccagtgnccaacgttccanataggncagggtcntgc

SEQ ID NO: 358 - DNA, 293 bp, Homo sapiens
  misc_feature (55)..(55): a, t, g, c
ttttccaggatttaatatggtgcttttaagaagagagccaccggtctcagcttanaattacattttcacaaattaatccnaaattttacgtatgaataaaaaggagtanaacaatacatagnaaatgaaattggagaactgatttaatactaaagttctgaataaaggngtgcactttatgantgnttctatctttttgcncaagttggntactccagnttnccatcccnacatgttgntcgccgangtgtgagaacgtggntgnaagncgntnntncccgnttgaacgcaaa

SEQ ID NO: 359 - DNA, 500 bp, Homo sapiens
  misc_feature (1)..(1): a, t, g, c
nttcagttgagtaccaatgccagaacttgtatcaacttgagggtaacaagcgaataacatgtagaaatggacaatggtcagaaccaccaaaatgcttacatccgtgtgtaatatcccgagaaattatggaaaattataacatagcattaaggtggacagccaaacagaagctttattcgagaacaggtgaatcagttgaatttgtgtgtaaacggggatatcgtctttcatcacgttctcacaccattgcgaacaacatgttgggatgggaaactggagtatccaacttgtgcaaaaangatagaatcaatcataaagtgcacacctttattcagaactttaagtattaaatcagttctcaatttcattttttaatgtaantgttttacnccctttttattccatacgtaaaattttggattaattttgggaaaaatgtaattatnagctgagaccggtgggtcctcttcttaaaaggcnccatattaaaatcccgggaa

SEQ ID NO: 360 - DNA, 284 bp, Homo sapiens
gcggccgcctgcgcgcacagccccagtggtcggatcctgggcggcagagaggccgaggcgcacgcgcggccttacagtggcgtcggtgcagctgaacggcgcgcacctgtgcgcggcgtcactggtggcggacgagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccg

SEQ ID NO: 361 - DNA, 410 bp, Homo sapiens
cagctaattttttatttatttgtagagatggagtcgcgctacgtggcccatgctgatctcgaactcctgagatccaatgatcctcccacctcagcctccccttctgcatatagtaggtgctcaataaagaccaaccagatgcaggagtggatgacttcattgctcgggactttgttgcttgggtgaccctgaccttcaggccccggcaccctaggccaggacgctgtcgatccaggccgcatagctcgccacgcgggtgtagatcccgggcttcttgcggttgccgaaacgcgcgacccgaggtgaccacgccctcgagcacgcccccgcacaccagcggtcccccggagtcacccttgcagctgtcccggcgattgctctccgcgcacatcaagcgctcggtgatggcg

SEQ ID NO: 362 - DNA, 393 bp, Homo sapiens
aatttttcccaacattttattattaaaaaaagttcaagaaaacagaaaagttcaaagaattgtacagtttacattcatctgcccaaaatctagaccgtataattaattttttattgtatttgctttatcacatcacgtatcaatcattcttatttattgatggatttcagagtaagttgcaaaaatgcatacatttcacccctaaacatttcagcatgcatatcctcaacttagagtctaatatttgtttatagctccttttttgaggtaaaatgtacatacaatgaaatgtatataacttaagtgtccaatttgatgagtttggcaaatgcatacacttgtgtaacccaaattcctattgtaatatagaacatgaccattaccttagaaact

SEQ ID NO: 363 - DNA, 296 bp, Homo sapiens
aaacccaaataaataattttattaaagaagggtggattaagtaaaacagtcagtaggaataaatgaacaaaccagaccaaaaaaacccccagaactcaattatatttgtttataagagggccctgagatacaaagacactcaaaatataaaaacacaggaagagaaaagcaaacagatgaaaataatgtactctataaacactaaccgaaagaaaactaataacatgaataaccaattaaccaaccaacctaactgatacatatacctaataaatatagaatacgcagagacaatt

SEQ ID NO: 364 - DNA, 464 bp, Homo sapiens
attcggaacgaggagagagactcatatattatgtaaataatggaacaaacaaacagattttttaaaaatcataattaagatgttgaaatgtagaaaagatgaacaaagtgtaagaaaaaatagtacccacagaaatatctatataaaaaattaaatggacatcttatacctaaagaatacagtattgggaataactcattgcatgggtttgagggcagacaagagccaacagaaaggagtaataaacttgatgaaaaaatggaaaatatccagaataaagtacagtgagaaaatacaaaaaaaatttagagcattagagataagagatagagcctgtaatccaggctgaagtgcagtggtgtgatatagctcactgcaacctcgacctgggctcaagcgatcctcttgagtagctagggctacaggcacacaccatgctcaactaatttgtttttgtagagatg

SEQ ID NO: 365 - DNA, 475 bp, Homo sapiens
gccaggcaggaccacaggtttattggggactccacgcacagacgcttatggcatcacacgacaacggcacggttactcgggacacacacggtggcctctgcccacagccagggcccagaggcagtggggtgcagtctcctcccttgtggcccagacccagctgggtcccttcctcctaggcagctgagggaaggactgctgggttggccacgggcctgggaaggggaagcgagcaggcgagtccaggaggggccggggcgggtgtggggctcgcctgccctcactccagggactgtagcatcagcagctgtttcagcaccttcgtctggctggtctctcggatttcgccagccaggaacatctcgtccacgaccgtgtaaaccttgtagaagttgaacaccaagtccagttcacagacattgtggaaatattcgcttaagacctccacgaaattttgaaatggctccacgtaacc

SEQ ID NO: 366 - DNA, 435 bp, Homo sapiens
gtccttggtcatgtcaggagggacaggacgttgtatcaatatgtctggttcctcattaaatttgcaactactcttttagcctttattgtttattttctgactctgtttttttttttctttagccaactcacgccatgactctacttttgcttctatgtttattagttggcattctgtactaagacagctttacccttcttgcctacttatttatgtcagtatgagctcagaagttcttattcaggggcttataatacagtactcttattattttatggttacacagtcccaactttgtcctgtgggaaccctttcaggctggctcttatgttctggtgacataatctcatccttctttaagtactttctttctggtattaacaatatgctccagactcattcttcccgagccccagccccagaatcagctttttc

SEQ ID NO: 367 - DNA, 321 bp, Homo sapiens
  misc_feature (156)..(156): a, t, g, c
ggcgtagagaaaatatagagccactttactattaaaaatgtattttccaaaagcaaggtagttgctgtcccaaaaagccgaaagcctctagtccctgcggaacgggctggatggggcttcagtctgacacagccaggcggggcagccctcggcggngctcggattctctgggagatttgatagagctccatcgttgccctcgcatcttccaccgagctgtgtccaagcaggctgttctggatgctcttgtgcaggaggcgctcactcagcacccgcaggagacacgcctgcagtggtccagcttggcctcacgccacaaca

SEQ ID NO: 368 - DNA, 337 bp, Homo sapiens
ggctacacaatctacgacacgtccactgacaggctgttgtggcgtgaggccaagctggaccactgcaggcgtgtctccctgcgggtgctgagtgagcgcctcctgcacaagagcatccagaacagcctgcttggacacagctcggtggaagatgcgagggcaacgatggagctctatcaaatctcccagagaatccgagcccgccgagggtgccccgcctggctgtgtcagactgaagccccatccagcccgttccgcagggactagaggctttcggctttttgggacagcaactaccttgcttttggaaaatacatttttaatagtaaagtggctc

SEQ ID NO: 369 - DNA, 410 bp, Homo sapiens
ttttttaggataaacttgccaggctttagatctatttgttcctttccatcccccaaaatcaaatggctgctccaggaaaatgttccccttgtggcagggtccgggagaaaagagagaagcgacaaaaccaaaaattaaaacgaccgaagtcccatatgctccaggaatatgtcctggagatgggagtggagggcagggggagaatgttgttgaggtcaaaatttttgaagttttaagtcctatatcttgacatcccgagtataaatgcgggtaccagacacagtacaaacgttctcaaagcccagttacgtattccaaaccaaacgcgggctcttgaagggtgatgaggtagggatgaaatccaggatcgcctgaagaccatttcttcctctcttagggacctgctggtc

SEQ ID NO: 370 - DNA, 434 bp, Homo sapiens
attcggcaccggggaggtttttcctcctggaccgaatcagctggagaccacaggtcctaagagaggaagaaatggtcttcaggcgatcctggatttcatccctacctcatcacccttcaagagcccgcgtttggtttggaatacgtaactgggctttgagaacgtttgtactgtgtctggtacccgcatttatactcgggatgtcaagatataggacttaaaacttcaaaaattttgacctcaacaacattctccccctgccctccactcccatctccaggacatattcctggagcatatgggacttcggtcgttttaatttttggttttgtcgcttctctcttttctcccggaccctgccacaaggggaacattttcctggagcagccatttgattttgggggatggaaaggaacaaatagatctaaagcctg

SEQ ID NO: 371 - DNA, 519 bp, Homo sapiens
ttttaaatgtaaattgtttattttagaggccattagttgcttttctgtgaagcaacaagaaatcttactgaccaggctaattttttaatgttatgctaatgttgtatcccttccatattctgtgtttcatggaatatcaagatgccaaacaaccagataaaactgacaacataaaccacaacatcagagctagcaagagatgggagaataaaatggcttgaattactaggatgtagaactaaatgcttggaatgaagctagtcttcctatctttaattttataaaatcccctgaacactaagaatggatttataacttaagacaatgccctggatggaaaatggataaggatgatttcataccccgagtgcctggtgggcaagagcacttgaaatggttcacaaggtcaatacaggtgcctccattctggcagggctgattctggcactcatccacttcatactcacagttgacaccctgatagcctgggacacactcgcatctgtaatcaccaatgaa

SEQ ID NO: 372 - DNA, 205 bp, Homo sapiens
  misc_feature (64)..(64): a, t, g, c
tttttttttttcacttttcgttatttttattgtttgacaagcatttacaacgttccattcatanacctaaaaacataaaaatagaccttctttcagcttataaaagaaatatataagaacttttgacatagacacgtcatgaccttatgtacaagagacaatggcaccctntccaagcaccagacttccgagtgttttcagatgg

SEQ ID NO: 373 - DNA, 488 bp, Homo sapiens
  misc_feature (412)..(412): a, t, g, c
aatgaaatgtgtcagcttccggtgttctgactgtacccttccctcttccatgtctgagaatctccgtgtattttaagaatgtgtgaggagagggtggcgattcatgtttcaatgagcctcttttttttttttccttcctgttttggtctatggctggtcttactctgtgtccatgttcggaagctctagttttgcatagaattatagagatgccaaactctttgaaaagagatccaaatttatcgcttgagagaaagaaaagaaacactattttttgtattttacctgagatacaggggcacaaataggatgaggaattttacagtgttagtgtatgtatccctgagcctaaaaaatgagggatataaccttttacagaggaggagtgagggcgtgggtgggtttttatatnttatnatatggaaagggcccagccaagcttcatgcgnaagggatatnacttttncttcccaaaaagcggggttttt

SEQ ID NO: 374 - DNA, 320 bp, Homo sapiens
  misc_feature (28)..(28): a, t, g, c
ccgttacaaatagcaccgagcccttggntgncccctgtggagagggatccccctcctggacccccacctcctcccctgagctacccatgtagtgtctgggagctggcacacggcccagggtccctctggaagctgggccaggccaaggggttaggcccaggcccttcgtggggtgggcccagggcagnctgtggcagatgtgcaggtaggtnagcggcagctntcagatgagcacantggccacgggnacgcgggtggagcggccccttgggancccacaatccggtcgtttgtgggaccctgnttcgtgcagcaggccc

SEQ ID NO: 375 - DNA, 377 bp, Homo sapiens
  misc_feature (41)..(41): a, t, g, c
ggagagaccctcctgtccatgaactgggccatcgtggcgancattctgctgtacgtggtgatccctacccgacgctccaccgccgaggccttccagatcgtgctgtcccacctgctgggtgatgctgggagcccctacctcattggcctgatctctgaccgcctgcgccggaactggcccccctccttcttgtccgagttccgggctctgcagttctcgctcatgctctgcgcgtttgttggggcactgggcggcgcantttnctgggcaccgcattcttcattgaggccgaccgncggcggcacagctggcacgtgcagggcctgctgcagtaagcagggttccacagacgtaccggattgtggtgccccagnggg

SEQ ID NO: 376 - DNA, 306 bp, Homo sapiens
  misc_feature (200)..(200): a, t, g, c
gctgccaaaagcctttaatatgccctggtcccaggctgtgttcatgaaagcggacacagcagtgcttccagcttcatggttcccaggttcaggttcctcccagcggaggtgggagggcagccctcacacctggcacccctgagtgcatactcctggaggaagtcgttgagctgggcacaggctgcccgctggcggttgcntccggcacaggcgttcagagggcatctcctcgatccagctattcgagtccagcaagtactngggggggnccctcccaggggcataantnggnccntccaganccat

SEQ ID NO: 377 - DNA, 449 bp, Homo sapiens
  misc_feature (2)..(2): a, t, g, c
antagaagtgacagtcaaaggccacgtcgagtacacgatggaagcaaacgaggactatgaggactatgagtacgatgagcttccagccaaggatgacccagatgcccctctgcagcccgtgacacccctgcagctgtttgagggtcggaggaaccgccgcagagggaggcgcccaaggtggtggaggagcaggagtccagggtgcactacaccgtgtgcatctggcggaacggcaaggtggggctgtctggcatggcatcgcggacgtcaccctcctgagtggattccacgccctgntgcttgaccttggagaagctgacctccctctctgaccgttacgtgagtcacttttgagacccgaaggggccccaacgttcttgcttgtaattttgaacttcggtccccaacttccccgggaagttgccttgggcnttttaaggcttgtncaa

SEQ ID NO: 378 - DNA, 420 bp, Homo sapiens
acaaaaataaatttttattaaatcagtagagtctgagcaggaagacagtacaaagaatgtaaacaatgtaaacatcactacattcagctcagcctgattgaatgctgaaagacaactctaaatgctctacgtgcccttactagcaagatacattaattctattttacacagacaacaaacacactctaattttgattaggcaaaagtattttatcgaagtcgatcccagcgccaaatactggcatcatcacaaacagctataagaatgctgctatccctgctaaaactggtttgtcgaatagcagcaccacatttatgatgagtcagtgttgtacatttggctttatgaggatcttctacttctaaatcccaaacataaagtttgccaacttggatgcccaatgcaagcatcttttgcca

SEQ ID NO: 379 - DNA, 430 bp, Homo sapiens
actattcttgggcgatttgattacagccagtgtgacatttggtacatgagcgttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattagagtgtgtttgttgtctgtgtaaaatagaattaatgtatcttgctagtaagggcacgtagagcatttagagttgtctttcagcattcaatcaggctgagctgaatgtagtgatgtttacattgtttaca

SEQ ID NO: 380 - DNA, 371 bp, Homo sapiens
gccaacactgacactttgctgccaaaagcctttaatatgccctggtcccaggctgtgttcatgaaagcggacacagcagtgcttccagcttcatggttcccaggttcaggttcctcccagcggaggtggaagggcagccctcacacctggcacccctgagtgccatactcctggaggaagtcgttgagctgggcacaggctgcccgctggcgttgcttccggcacaggcctttcaaagggcatctcctcgatccagttattggattccagcagttactggggttttccctcgagtccaaaagtgggcccaatccggccccatgttaaaatttctttcccaggtttcaaagcaaagggcacaggggtctcga

SEQ ID NO: 381 - DNA, 474 bp, Homo sapiens
gcagatgttctgtgttttacggggcaccaagtaagagcagactcttggccaccttgtgttctgctgaagtctgccagtgtgctgaggggaagtgccctcgccagcgtcgcgcccgtggagcggggtctgcaggacgaggatggctacaggatgaagtttgcctgctactacccccgtgtggagtacggcttccaggttaaggttctccgagaagacagcagactgctttccgcctctttgagaccaagatcacccaagtcctgcacttcaccaaggatgtcaaggccgctgctaatcagatgcgcaacttcctggttcgagcctcctgccgccttcgcttggaacctgggaaagaatatttgatcatgggtctggatggggccacctatgacctcgagggacaccccagtacctgctggactcgaatagctggatcgaggagatgcctctgaacgcctgtgccggagcacccgc

SEQ ID NO: 382 - DNA, 329 bp, Homo sapiens
  misc_feature (311)..(311): a, t, g, c
gtgatatttttggcgttttattaaaaatggaaaaagttgttttagtgagcactcatggtgctttctcccctcctcccccaggcagaccccaaaagtgtcagggaaaggcaggccccctggaggggtgcggtggggctgaagcaggggctgctatggctaccagaggtgagctggtgatgtgagctgagttgtcacatgacactctccaccacgacgaccttgctgctctcggacactggggtcagggtggtcaggcccctgacatctgcgtcctgagctcggtactccaagtggtggaggccccagatggngctgggtcaggtgctgcc

SEQ ID NO: 383 - DNA, 248 bp, Homo sapiens
tgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctga

SEQ ID NO: 384 - DNA, 385 bp, Homo sapiens
tttgactattaaacatggctttatttaagtttgggcaaaaagatttctgagttaatctgggagatagagaaaataagaatctgtaggattttggtttttgttttatatatacaactcttctctgctgaatgtaatatagatggaaaaattgtgatgctgctatcaagctgttgaaaacagtctgctcacccagatgggcactcatttgaaagacaggcacaccctgtcatttcccagtagccagccctgcagttcttacacctggctttatgcaagcacagaaggtgaggcataagccaattaataacttgtgtgattccaggccgtctctacccccatggccaaattctggactgaagattgcaaacccacaggagactctgct

SEQ ID NO: 385 - DNA, 465 bp, Homo sapiens
  misc_feature (356)..(356): a, t, g, c
tagacacctgacagaactcctggccttgatgtctcagctggaacaccagaacagtaccatctactacggcttttgcatgtagcagcaaagaaaaaacaactcgagcttgcaaataggaaggactcaatcagtatttgtgaatgagtgaattaatggataaatataaagagaagggagtgaacagatctgtgggattcttgggcattggaatcaacaggacatgatgattgcttggataacagctgctggtagttcagaagtgtattcctttcctaattgggcatttgaaggattcaacccataatgacatcatcctaaacatcctcatagagatagcagtctatgagccagtggtnttgaacagttttcttccaatgctgaaagagattggtgagagattcccctacctcactggacagatggcaaggatttatggagctgttgggcatgtggatgaacaaactc

SEQ ID NO: 386 - DNA, 472 bp, Homo sapiens
ttaagaactgaaggtttaccatcttgatgtaggccttgccgtcctgaagagcactcgggctcatgtgtttcattcactcacatgttaaatatgaagccctatgtacaacatagcctgtgggaactgtggcaggggtagctgtctccatcctccagaaccttgtcacaatcaagaataggaaatgagacgcacaaatcactaccacctgtcgaagtggccttgcagtgctaaatctaagtgctgaggcaatttgcctgcagtccttgccacttcttgttgattagtccagcagcccccagctgggaggcaattctgttcaccctcactcccacctcaggacatctggcaatgtctggaaacagttggttgtcaccacttggggaaaggggagtgtgcttttggcaggtagaggcctgggaggctgctccacatcctacagtgcacagaacagtctccccaaccaagaataatc

SEQ ID NO: 387 - DNA, 422 bp, Homo sapiens
tagccaaagcggacaaatctgtcccctcccctttgttgtaaccacacattcaaggcttccttcagtccagccccatctcccttttcgttgcctccctttcactactccaaatgagcttgccatactttaaacgggtcttgcactttcttatcccttctctggaatgtcatttctactgaagaaaatcctactcttccctcaagacccttctttaatcccctcttgccatggaaacttgagattttcattgaggagctaagttggaaaatataaactagagctgagtctctaccctggcactattgacatttggggccagattattcttggttggggagactgttctgtgcactgtaggatgtggagcagcctcccaggcctctacctgccaaaagcacactcccctttccccaagtggtgac

SEQ ID NO: 388 - DNA, 418 bp, Homo sapiens
aggtgaaagtttgctatttatttagtcttagaaaaacactgaaagaaaaaggcaggaaatgtagtacgcatgtggaagaatggagctggccacatgtagttttagcagctgcagaggaaactggctgagtctaaggttacatatttctgctcagtaagggtatccatggtgataactctcatgatgacggtacatccctggtaaccatcctggtatccttggtggtaactatggtaaccatagcctgcatactcatcttctgggcagccatccccagtgactgctgaatcgccatttcctgtctccgaagttgcatcgaatcaattaaatcgtacacaatcaccatggaccacattggggaaggataccaggatttgacatttccgtcttttccgactagaagcatggagaagtactc

SEQ ID NO: 389 - DNA, 295 bp, Homo sapiens
gcagctctctgcactcagtggtcaggcgtgcaattttggtctgcgccacataaccattctgaagcttttaggcgttggagaggaagttgggggagtgttagaactgttcccaattaatgggagctctgttgttgagcgagaagacgtaccagcccatttggtgaaagacattcgtaactattttcaagtgagcccggagtacttctccatgcttctagtcggaaaagacggaaatgtcaaatcctggtatccttccccaatgtggtccatggtgattgtgtacgatttaattgat

SEQ ID NO: 390 - DNA, 443 bp, Homo sapiens
  misc_feature (352)..(352): a, t, g, c
acagtgacgcttcacagggctcctggatttggatttggaattgcaatatctggtggacgagataatcctcattttcagagtggggaaacgtcaatagtgatttcagatgtgctgaaaggaggaccagctgaaggacagctacaggaaaatgaccgagttgcaatggttaacggagtttcaatggataatgttgaacatgcttttgctgttcagcaactaaggaaaagtgggaaaaatgcaaaaattacaattagaaggaagaagaaagttcaaataccagtaagtcgtcctgatcctgaaccagtatctgataatgaagaagatagttatgatgagggaaatacatgatccnagaagtgggccccgagtgggtgtgggttaacagaagggagttgaggaaggttttnggccnaggggttaggaagttgcaagtaggagagagg

SEQ ID NO: 391 - DNA, 355 bp, Homo sapiens
  misc_feature (118)..(118): a, t, g, c
gggccatcatcttcttgaatattgccatctcttgctgccaaactatcttgtgaaatttccttaacaaatatatggcttgcaatcgaagaccatattcttcatttttccgggatttcancagtgtgactttagnagggttagganggntggnnggagncactganccgcctgtttnaccgnggggnnangntcctntctctactngnacttctatccctcggccaaatcttctnactccttctgttaaccacaccactccggccacttcttgggatcatgtatttcctcatcataactatcttcttcattatcaggatactgggttnagggatcagggaggatttaatgggtattt

SEQ ID NO: 392 - DNA, 512 bp, Homo sapiens
cagagatggacagagaactttattttggattgtggatgtggacttttttgtacataaataagaaaaaccaaaatactccaaagatgacttcccctgcctcctactccagtatgacagaggaggatgtaaggccttagccatgatctgcaggggtctgggagtcaggcccggcctattgcttgggtctctctctatttatatatctaagttcacagtgtttcttattcccccctaagcttctagaggctcatggccctgtagttaggcctggctcattctgcacctttccagggaggtggaaggaccctgtgccctccttcccaatcttctttttcaggctcgccaaggcctaggacctatgttgtaattttactttttatttctaaagttgtagtgaagctctcacccataataaaggttgtgaatgttctgtgagtgtcatggagatgggctagggaggggattttacacttcactttccagacccctggtttgggggaagagggtccatg

SEQ ID NO: 393 - DNA, 425 bp, Homo sapiens
gcagtggctgccgcagaggatgggacagagacacccggacttctcccccaggacctaccaccctctgtccttgcagtggaactagaggaggatggaagagcccgacctctggtcctgcacatgtccaccctcctgcggatgatcccaaccatggcagaggacaaagagagagtcagcactggaaccaagtttgccctcaagctctggctgcaacatctgggcagaacacccccaccgtgtaaaagtgcagcttacctggacccagggccagcaaaggaagaatggaacatggaccctcttcccccaaaccaggggtctggaaagtgaagtgtaaaatcccctccctagcccatctccatgacactcacagaacattcacaacctttattatgggtgagacttcactacaactttagaaataaaag

SEQ ID NO: 394 - DNA, 291 bp, Homo sapiens
caaaagacaacatattttatatcaaacaagtttgaagagccctgaattgcagcattctgtaacataaacaaacaaaaagctggtataggatttattgtcaaaggcagaatttcttcaggcaggtaagtaaggaggtggtggttctttttcaggcattttcacggccatttcataggttggcaaaacgtactgaggaggtgcttcaaaggcagggtacacagcaatctccggcacgtttcggttgttgatgtatttatagcagttccaaacacagttaattagataagccct

SEQ ID NO: 395 - DNA, 588 bp, Homo sapiens
aagacgcgtcgttgggttttggaggccgtgaaacagccgtttgagtttggctgcgggtggagaacgtttgtcaggggcccggccaagaaggaggcccgcctgttacgatggtgtccatgagtttcaagcggaaccgagtgaccggttctacagcacccggtgtgcggctgttgccatgtccgcaccgggacgatcatcctggggacctggtacatggtagtaaacctattgatggcaattttgctgactgtggaagtgactcatccaaactccatgccagctgtcaacattcagtatgaagtcatcggtaattactattcgtctgagagaatggctgataatgcctgtgttctttttgccgtctctgttcttatgtttataatcagttcaatgctggtttatggagcaatttcttatcaagtgggttggctgattccattcttctgttaccgactttttgacttcgtcctcagttgcctggttgctattagttctctcacctatttgccaagaatcaaagaatatctggatcaactacctgattttccctacaaagatgacctcctggccttggactccagctgcctc

SEQ ID NO: 396 - DNA, 328 bp, Homo sapiens
tttttagccatacacatgcctttatttagatcagcttttttcaaaatgcagccaaacttatgagttggacagcccaaagtaaccagccctattccactgagttagtttaccccacagcagtagaacccagtgctggtttggttcctggcccatggtgggacagcgtgaaggtgatggagggctctagcacaaggaggtgctgagtgccaccggaggtcatatctgcagacagcctcattgctttactaaagggtcaggctaggcatgaccgatcaaggatgggtagggggtaaaatggaggaggaacatgactgatgaggtgcctgga

SEQ ID NO: 397 - DNA, 457 bp, Homo sapiens
  misc_feature (3)..(3): a, t, g, c
aannnantgttgacctgtcactgtttattatttcagcactaaaactgaggngcctaactgctggctcttcttccctttgtatttgtgtaaggngcactgcactcccataaaaggttttaaaatacaaaatgtacaagaacacacaattccaagtgctgtaaacataactgagaaccagttcctttactaaacatccattttataaaacacaaggtttcaatttgagcccatctgngccttaaagntccattctgaataccaaaaacagggcttcacagccaggnccagaagaggtctggngataatggctggccctgggtggggatagtttacanccgggnagcagcaccacacatggnacccaaangacatgttctttttaaagctgttttnagccatgttttctctgtggcatctccagtaagcnggagggtaccctttcctttcctcaanccaa

SEQ ID NO: 398 - DNA, 394 bp, Homo sapiens
  misc_feature (374)..(374): a, t, g, c
gtctattcttcagcagatcactgtggcccctcttggagctgggtgttttcaaattttcttgaaaagtgagttgtgtcacagaatgtggatagtgagatttgcttctgggcctgtggttggacgcccacagcttcctttggaggggcaagaagttggcctctcggttgacattccagagaaatggccagacttgcccctacccttttgcccctggtgtagctgcctcacatgaggttattgcaagagttccccttgcaataccctttgtgccctcaagaattaaaagcctctatgatccagagttgctatgcaccaaattttgatgccttctaaataccttgatgcctgtagcactagaaatgctttcctatttnaaaatgaaacatttaatttt

SEQ ID NO: 399 - DNA, 541 bp, Homo sapiens
ttttcaggagattcttaggagagttttattcattcattgatccagtatttacaggggctagaggggtcaagctgtgctcagcccagaggcagctgccacacttgccagcaccccccactcagtcactatgtacagataaaggggcctgcttggatcacctttttcaaagccatctggcagaggccatggggctgtgttggggcctgggctccagaggcactgctgggcccattaccccttggcatcaggtcctctggaacacaggggctccaacgggttgtcttgatcctgctgtcccccaccctgagtgccttgcagaggctgaggaaactggagtagcaggaagagctgagtggtgccagcttcctataagcaaccctgtctctgctacccctgagagggagacatggtaatactgaggggctggacagaggctcttctgagctcaagcgccagggacagagacctagagcccaatttaggcccatagctagggccacaacactgaaggcgaagagcctagaaccttgctatggagagc

SEQ ID NO: 400 - DNA, 468 bp, Homo sapiens
  misc_feature (2)..(2): a, t, g, c
tnnttttttttttncacctttccctaatacttnatnggtnacctctaggcctgtgtgcggctgggtgggcttgggggagggcgtcactattcagcttctaggtggaggcatgagaaggccttggctaggccctccagggtcccatactgtggagtttggaggggcaggtctggcctttcctgggtcagcatagggcacccaggtgggggnacaggtggacacccagcacaggcacctaggcaggggcacaagctcantatccnttagccagcctaattgtntttggagaaatattccttgctgtcatccacgttgggtttaatcgtgtcactgccaggtttccagccagcgggacaaantttccccatgttcgtttgtgtattgggaaggcctgggaccagccgcagagttnatcccacggagngtcccaaaggnaaatcattaaacagtgattttggcnaaggaaaa

SEQ ID NO: 401 - DNA, 394 bp, Homo sapiens
acttcaaggccacagcggtggttgatggcgccttcaaagaggtgaagctgtcggactacaaagggaagtacgtggtcctctttttctaccctctggacttcacttttgtgtgccccaccgagatcatcgcgttcagcaaccgtgcagaggacttccgcaagctgggctgtgaagtgctgggcgtctcgggtggactctcagttcacccacctggcttggatcaacaccccccggaaagagggaggcttgggccccctgaacatccccctgcttgctgacgtgaccagacgcttgtctgaggattacggcgtgctgaaaacagatgagggcattgctaacaggggcctctttatcatcgatgggcaagggtgttcctttcgccagatcaatgtta

SEQ ID NO: 402 - DNA, 446 bp, Homo sapiens
  misc_feature (17)..(17): a, t, g, c
gtttgttgttttttttnttaagtggaantnggctttccagctttnttgacnctccctgtctntgcanttnaantgtacatgccntttanggngcccnacaagcttngggancagtcncnaacgcatccctatntgnagntgggcgaaggcgcactggagcaggttaagtcccaggcggcaatggtgagccaagcgtgggaacagcccaaactaaagtcggggtctctgtgcggcggccgggccacttcggcacttcagtgtttaaacttctgcatgcagtgtaaagacagacagctggccacagtgctgccctgcagtaaagaccagccaccgtgaccggctctcccctgcctccctcctcccagcccacagtcagacggagcctctgtggcaagctcaggcccagtccagggccctcagagtgtacctcgtgccgaattcttggc

SEQ ID NO: 403 - DNA, 406 bp, Homo sapiens
  misc_feature (25)..(25): a, t, g, c
tacactctgagggccctggactggncttgagcttgccacagaggctccgtctgactgtgggcnggnnggngggaggcaggggaganccggtcacgtggctggtctttnctgcagggcagcactgtggccagctgtctgtctttacacangcctgntaagtttaaacnctggcttgccganntngccgtgccgccgcacagagaccccgactttagtttnggctgttccacgcttggctcaccattgccgcctgggacttaacctgctcaggcgggccttcgccagcntgcaaatagggatgcgttagatgactgttcccaaagcttgttgggctccttaaatggcattgtacaatttaagtgcaaagacagggagtgtcaataaagatggaaagccatttccagtt

SEQ ID NO: 404 - DNA, 396 bp, Homo sapiens
atgactgacagattatcttattttattatttaacgtatctcatgttttcttttaaggtctctattgagataaagagttccagggaaagaaaggtcacagtgctcggtaaacaacccagcaaacggcggctctgctgctgcgtgggccaagcgggtggtctggggctgtgacggcctctcagtgtgtgatgaagtttcagcgccctccacagtggcagtgcatgcttggaatcccgcttccggttgtgttgttcattctggctagctgagtttcagttagttcatgatctgtttaaaccagagtgagtacctggagaccttggtgacaaggaccatgtgagcctgccctcctactggctgcgagcccaggacccccgtgagaaaccaggagcctctg

SEQ ID NO: 405 - DNA, 359 bp, Homo sapiens
  misc_feature (5)..(5): a, t, g, c
ttctngctgggtnctgttgtattttttgttttgtttttcaatccattgttaattctggaaaagctgggctggcaggtcctgtggtgaatatgttggggctgtggcctggggttcttntgggaggccaaggcatccatcctcatagccaggcggggctgtcattgccagtacaaagccagctgcagtcgccacttcatctcagaggatgcgcaggagcgcaggacaaggaggtcttccagcagaacatgaagcggcggctggagtcctttaagtccaccaagcacaacatctgcttcaccaagagcaagccangaccccgcaagactggccgcaggaagggcatgtcttccctcagggac

SEQ ID NO: 406 - DNA, 454 bp, Homo sapiens
  misc_feature (9)..(9): a, t, g, c
ttttttttnnctcngntntagaacactgcagctttattganagaacaaccgcgtccgcaactagaggtnacatttgggggcttggaggtnaaagagaggcaaatggggaacagaggctggggtggagacagacagcaaggcaaaggggtgaggatggggctgtgtgactaaaaaaacaaccccaatcatttcacccnttaataagggggtctcctgttgtgctttnaccttggtggcggggtgggctggggtggtcagacatntgggcctgggcatgagganttttggcttttctcccaagtctttnaggtctcagggctggtgcagccccttccctccctgacccttctcctggggctgggganaaaggccttgggtnagctggctactctaggggcaggaagaatgcatgggagacctagagggccaccagagttgggctgagatggtgatt

SEQ ID NO: 407 - DNA, 511 bp, Homo sapiens
  misc_feature (338)..(338): a, t, g, c
cggaacagggaggcattttgtggccttaaaacggggtgttcagggtcagccctcagggtgctggggcccttccatcccttgtccccttcaggttggtgaaaaggactccgggggccaggtgctgtatagcagcttatggaagtctcagctgggctatcctgcccttgggagcacagacaggctcctagggtgctgagtgaagcctgaagaccaagccccctcctcctggaatgctccttccacccctacctctcagagatgggcctgacacctctttctcattcattctccttttccctgtgctctgggaaagcccctggctcaggctgttcagagtnaggatggacctcaaaagtttgatcccctcgtgcagatgaagaagctgaagcccagagtgcggaaggggagttggcccaaggttcacacagctaagttttgagttagagccagtctttgggagagccaataattaanagccntgggggtgttcanaacgtttgttaatgcccgt

SEQ ID NO: 408 - DNA, 343 bp, Homo sapiens
  misc_feature (155)..(155): a, t, g, c
tttaactaaaatggtcacttttaatgggaaccagaggtatagttacaattacatagtccgacggggggaaacccttgggtgatcaggaatggggaaggttacaaaataacgagggtaacacttgggtacaggacaaaaagctgttccagaacctngggagccaggctaattaatacggcctctccctgcgatcctatctgtgctcacccctggaatccatcttgccagggccaaagccacctctntccccaccaccccngccccctcggaagcctccacggtccccgcctgcanccccggtangccgcctncgatcataagcntcctctngncaccactgacg

SEQ ID NO: 409 - DNA, 377 bp, Homo sapiens
  misc_feature (54)..(54): a, t, g, c
ggcggtggaggacagcagcgagctggtgactggaagtgtcctaatcccacctcnagagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggnnccnggcggggaccgtggaggcttccganggggccgggntggtggggacanaggtggctttngcctggcaagatggattccaaggggtgagcacagacaggatcgcaagggataaggccgtattaaattagcctggctccccaggttcttggaacatgccttttt

SEQ ID NO: 410 - DNA, 521 bp, Homo sapiens
aaaaacaacaacaaaccactttaatggacacataaaatccctgggggtctcattgcaatgcagatttggattaaacagtctgggtgggtcctgagaccctgatccccttgcttttcacctatggccacattttgagaagcaaggccactcaaagggcccccatctgtgtgaatcaacccctgctccatctgcccctataaatgacaggagtgggggtgtccccaggtctgagcctggagggggttgcaggcttcctccaggcatggaagtggagtgagaggaggctgtagttaaacgggttactctgtcagcagacacagaggagaccagaatagcttttatttcacatgaagctaaggaaggaagtcgagaggacagggctggttctgtgggcgctgctggcaggtgcaccacctccaactgctgggctccagtccagggctcagaagccatctccgagaaggtcgggagcggggctgaagggtcccgggggcaggttccaggccagctcctccagcaag

SEQ ID NO: 411 - DNA, 567 bp, Homo sapiens
  misc_feature (360)..(360): a, t, g, c
agagaagaccgtggatcacctggggacagaggtgaaaggcctgctgggctgctggaggagctggcctggaacctgcccccgggacccttcagccccgctcccgaccttctcggagatggcttctgagccctggagctggagcccagcagttggaggtggtgcacctgccaggcagcgccacagaaccagccctgtcctctcgacttccttccttagcttcatgtgaaataaaagctattctggtctcctctgtgtctgctgacagagtaacccgtttaactacagcctcctctcactccacttccatgcctggaggaagcctgcaaccccctccaggctcagacctggggacacccccantcctgtcatttataggggaagatggagcaggggttgattcacacagatggggggccctctgaattggcctgcttctcagaatgttggccataggtnaaaagcaaggggatcggggttcaggaccancagaatgtttagtgaatctgnatgaatgagaccccaggatttatgtgtccattaagtggttgttgtgntttaaaaaaaaaa

SEQ ID NO: 412 - DNA, 332 bp, Homo sapiens
tttaaatggagtcggggtcccgctgtgttgcccaggctggtctggacctcctgggctaaagtgatcctcctgccttggctgattttgttaatttttaaatatcttcattttgatctatcattaatggagaaatattccgtctaccatgtatataaggactttcagcagatgaaatgcttaaatagtttaagggtggaaatgaatacagcaattagaaataaggggttgataaaatatgcatttagttttgctgttacagttaaagaagcattttttaagaaagactttctcatttatctactttgtgcaataaaacttaatttttgttgttg

SEQ ID NO: 413 - DNA, 431 bp, Homo sapiens
ctacaccaggcacttctgaagcggtccaaagagaaacgttccctgcccgtcgtagctaggcacgataactccgtttaatttctcgagcacaccgatgcctatctcgtttatcattcagcagttaagtaattcatcacttcatatttttaccaaagtatatttacaagtctgtttatctggatctggcaaactggaaaatgtttgaagggatactcaggctttgatccatgccaacatgaaaggactccaagcacatttaagtaggctggtcttttcatcatgcatgattttttttttagtgcaaatttagactttagtttgggaaagagaacagtatacagtatcctttgtaagataaatcacaacaacaaaaattaagttttattgcacaaagtagataaatgagaaagtctttcttaaaaaatgcttct

SEQ ID NO: 414 - DNA, 474 bp, Homo sapiens
tatgctatggtttagctctttatttctacaattaaaagcagtaaaaacttaaaaaagaatttttacatcaatagaacattaactacagttggatttttcaatacctatgttcctcttccctctcttgattaacctgaagttgatattctttctgtctacttttaatacatatttatgattccatagataatactgtatatattagccattccctattttatcagtgacaaaaaatttgagagaagagacacagataatacttggagttatagatccagtcatagatataaggaaagttattagaagtatctataaaatatcaacagatcaaaaacactgccctttttttcttgaaatgagggtactaattgatgggggagagggagagtataaagggaacactagagcagtgtttctctctgtattaaaatcacctggggagctctttaaacgtccttgttctgggccacagat

SEQ ID NO: 415 - DNA, 375 bp, Homo sapiens
gaagatcatactgcattataaatttctgtaactaatagtaatcaacaattgtctattttgttctacccatgcaaacagactctagcactgtccaatcctgtacccactagctacagcacaatttgacaactgaccacttgaaatgtggctactctgaatctaatttcagttaatttaagtgtaaacagccacatgcagctagtggctactgaattgcacagtgcaggtctagactcctaaagcagtgcttctttaccttggtttcataatgggatcacctgtgaagctttacaatacaccgctgcctaggccccttgccagagcaattggattttaatctgtgggccccgaacaaggacgtttaaagagctcccc

SEQ ID NO: 416 - DNA, 374 bp, Homo sapiens
tttttttttttttttttaatatctcaaaaaggaagccacttgcttgatatcaaaagtgctgtggaaagaaaggaggggaaaaaaacccacaaataaatgtagaatctagtttattttaccaaactgtatatttttccacttaacatttctacattagcaatgatgtaactctccaacatttccttataaaaaaggcaaacaagaagtgacaactcatgctccaaatattaaaaaatatatttaaacatatgatcaagaagatttgggccgtgagtggtagctcacatctgtaaccccaggattttgggaggctgaggtgagtggatcacgaggtcaggagatcgagaccatcctggctaacatggtgaaacccc

SEQ ID NO: 417 - DNA, 425 bp, Homo sapiens
cccaggttcaagcgattctcccacctcagcctcccgagtagctgggattacagggtgtgtaccaccacgcctggcaatttttttgtatttttagcagagttggggtttcccatgttagccaggatggtctcgatctcctgacctcgtgatccactcacctcagcctcccaaaatcctggggttacagatgtgagctaccactcacggcccaaatcttcttgatcatatgtttaaatatattttttaatatttggagcatgagttgtcacttcttgtttgccttttttataaggaaatgttggagagttacatcattgctaatgtagaaatgttaagtggaaaaatatacagtttggtaaaatagactagattctacatttatttgtgggtttttttccctcctttctttccacagcacttttgat

SEQ ID NO: 418 - DNA, 249 bp, Homo sapiens
ttttgttggggtggattttattagcgggtctgcagtctcacgatcacagtggggcgggaccagcgcggcccattgagcgcaggcccgacgcgtctcccgctccttattggctgacagcagaggcgttgctgtaaacgtcaccgcgaccgccccacccagtgcacaaagcggagtgaagctggagttcctgggtccaagtggtgttagtccctgcgggcgaaattcctgagtacccgtcctgaggaggct

SEQ ID NO: 419 - DNA, 384 bp, Homo sapiens
tcaggtccctaccggcacatcacacactcctggaagcatgatcttcactcccttccttccacctgctgacctgtctgtctttcagaatgtgaaagggctgcaaaacgaccctgaggaatgggtggctgtgtctgacgccacggaggacccatccggtggcacaggcttgctcagggaacctgctcttctgcgagggtcttggaggagccggttccagagagccctggcatgtttcaccaagtgtttcaggggaggataccgggcactcggaatctgagaccaccggagccacagacacagagccatgaacggtgaccaagccccttctgctgctccatgcccctgctcgccgcagtgtcccagtcaccaggcctgaaataaatg

SEQ ID NO: 420 - DNA, 305 bp, Homo sapiens
aattttttttttttttttttttttaacagtatctccaacatacattttatttagtttaaaattccttaatacaagaaggtgttactccttaaccactgctgcagcctcaccggaccctcctgatgtggatgaagcaggcggggtggctgctggtcggctgtggaggcacctccaggcacagggcctagagggtgagaacactggtgacattaatgccatagctgtggcttgggacagggattgagaggcagctctcagggcacatgggcatcagctttcacaggatcctgttcccagtcaggctg

SEQ ID NO: 421 - DNA, 175 bp, Homo sapiens
  misc_feature (81)..(81): a, t, g, c
tttttttttttttttaggggtaagaggtagtacactttattgaccgggttctctcaacatgttgcaacctctggcaaagcnaatcctgggctttgcaccatcccatccaccaaagacagaagagaatgcatctcatgaacatccacgnggcctccagacagcagagcanaggggc

SEQ ID NO: 422 - DNA, 388 bp, Homo sapiens
  misc_feature (8)..(8): a, t, g, c
gccaatgncacggtttcatcatggaactccaggacggctacagcacagagacaggggagaagggcgcccagctgtcaggtggccagaagcagcgggtggccatggccgnggctctggtgcggaaccccccagtcctcatcctggatgaagccaccagcgctttggatgccgagagcgagtatctgatccagcaggccatccatggcaacctgtcagaagcacacggtactcatcatcgcgcaccggctgagcaccgtggagcacgcgcacctcattgtggtgctggacaagggccgcgtagtgcagcagggcacccaccagcagcttgcttgccccagggcgggcttttacggcaagctngttgcagcggcagatgtttggggtttna

SEQ ID NO: 423 - DNA, 223 bp, Homo sapiens
ggaaaacaaaaggaaaacttatttattcttagaggtgggaatgtggggagtggggcagaacaggtggtggccctgggagagggtcccaaggggcagaggttggggatgtctcagtaaagaggggcaggtcatgaatagagcctccacccccagcaggggttccttgggcccgcccaagcactgggctaaaacgtggaaactgggcattgacaaagtacagcgg

SEQ ID NO: 424 - DNA, 234 bp, Homo sapiens
  misc_feature (106)..(106): a, t, g, c
gtgggctggtcacaggccgaattgcccacatcccgctgtactttgtcaatgcccagtttccacgttttagccagtgcttggcgggccaggaaccccctgctgggtngnnaggctctattcatgacctgcccctctttactgagacatccccaacctctgccccttggaccctctcccagggccaccacctgttctgccccactccccacattcccacctctaagaataaataag

SEQ ID NO: 425 - DNA, 467 bp, Homo sapiens
  misc_feature (304)..(304): a, t, g, c
ttttttttctgtgtcccctcagcatttattttctggatggtcacaacgtattcaatgtgtccttgagacagccacccagcccttagggtggtccctgcgtgtcctggggcgggcgacttcctgagggtgcagtgctggaaacagagactgcttcgagcctggccaccttccctcctgtgccggctgcttctaggagatgctgtgggacggtgcccggcttcgcggtgactaccatgccctaaggagcagagcctccaggatgcccccgggctgcgctggacgcttaagaccccagcacctaatnacagtcagcagcttccctccactgacagtgatctgcatggtccattcctgggcggcctggggggcttcgnttacagtttcaacctgggctgggtttcccgntttccttccccagggggagattaagtgttcgtgtttnaaattttgggacaaattcaattggg

SEQ ID NO: 426 - DNA, 401 bp, Homo sapiens
  misc_feature (362)..(362): a, t, g, c
ccatcatcatcacctcccacagcatggaggagtgtgaggccctgtgcacccggctggccatcatggtgcaggggcagttcaagtgcctgggcagcccccagcacctcaagagcaagttcggcagcggctactccctgcgggccaaggtgcagagtgaagggcaacaggaggcgctggaggagttcaaggccttcgtggacctgacctttccaggcagcgtcctggaagatgagcaccaaggcatggtccattaccacctgccgggccgtgacctcagctgggcgaaggttttcggtattctggagaaagccaaggaaaagtacggcgtggacgactacttccgtgagccagatcttcgcttngaacangtctttctgagctttgnccaactgcagccgncc

SEQ ID NO: 427 - DNA, 415 bp, Homo sapiens
tttttttttttttttttttttggcttcctctttctttattgggcgctttgtagatgtcacgcaggtctaaaagttacactgctaaataattatttaaaaactgaccaggacgaaggtcctgtctcggagctccaaaccagaagcaaaaaggaatcggggcggtgggctggggggtactcctccaacatcaccaaaacccagaaaacgaggatcctaagctcctccgcaggccaaatccagggcttgggccctgggctaacccgcaggtgcctctgactgcatcacactcagagtaagataaccagcaaggggctggagggaacggccagccgagtccagacatggacagatgtaactggaaggaggacaggaaacagacaggtactgtccagctgtaggtaagagagtgcagcta

SEQ ID NO: 428 - DNA, 504 bp, Homo sapiens
tttcttttaaagcatttatttagaaaaataaccgttccctgcccgccttgctctgtgccatgggacttgagcatctttcttaatactcttctgcattgggcctgcaacccacagtggtggggtccagcacacgcccctcgaaacgctgcggcactgctgggaggcctcacgcccttccagtcgtgacagttcccgtgttcactccttgtccaggcctctgtctccgggtctccaggtggacaggggcctctgcgggatgggcgcagctctgccacagtctgtgtgcccctcccgaggctctgctctccccgcatcggaagctcagggctggaagcgcggggagctgacatgaggttggtggaatgcgtgggcgctgtacaccacctcggggggcgagggcgtgctggcggccagcaccgagcacaggggctcgtggctgttcagcaacgacgtgaagtacttcagttcctctgtgagctgcttgatctccttgcgtagagccgg

SEQ ID NO: 429 - DNA, 413 bp, Homo sapiens
tttttttgcactttcttatcagatttatagagacgtgaaactatgtaaataccagaacaagtaggggacaaaccccaacaagtaaaatgaacagcccttactgttcctgccatctatatcttcttgctcacggtggcctcagttccttatcattcgtcctcacctcacctctcagggctctcctgaaaggtctcagaggagcctgaccagctgccgctttcctccgaagggcgatgtggaggtgatcgcaagcaaagcatgctgggcgcaaagggtccggatcctcccatgcacctttcatgaccccgtgcctgtgaaggcttgtgaaatgccactgactccatcaaattcatcaactgtcctagtggctgtttaatacctcgaagctataaacggcttgatttgggcagatt

SEQ ID NO: 430 - DNA, 353 bp, Homo sapiens
tttttttatttataataaacagaaatttatttggcttagttgtagaggctaggaaattcaagaccaaggggccagcatctgatgagggccttcttgctgtgttcacccacggcaaaggtgaaggcagaagggaaaaagagtgtgtgaggaaaagagggagccaaacttgcttttataaccaacacactcctgagataatgggattaatcttttcatgagggcagagacttcacctaatcacctcattaggccccatctcccaatactattgcaagtttcaaacacataactttgggggacatattgaaactattgaaggtgataaccattgacattctttacaggagaagaac

SEQ ID NO: 431 - DNA, 377 bp, Homo sapiens
cagttaaataataatagttaatgaaggtgtgctacagaaaataatctggtgttcttgctaactttgcccttcactgttgcttaattgtgaacagccaaaagctatatgttatggcttattgtgtgaaggtaactaagaagtggtgttccatgacttcagagtacatccatgcggagtccattatttgagtttgacatttaataactttgctggaaaatctgtaaaaaagaaaaacaagtttgctagtgactaagccccgcatatgtgagtgaaagtacttcaggcacgctgcctcctggtaacagctatgcagggagtggaggacccacactgctacacttctgatcccctttggttttactacccaaatctaaata

SEQ ID NO: 432 - DNA, 542 bp, Homo sapiens
  misc_feature (503)..(503): a, t, g, c
ttttaaaatctactttaattctgttataaaatttataatgcagtttaaactatgatttctctccacttgatgatgtctctcactctgttcctttaattacgaagtctctgaagactctgaacttgactgaggaaatgttaaacagatacctcttcataattctgtaagtgtttgcttttaactttgaataaatgtcatatctaaacaaatattaaaaagtatttaacatctcatacagtcagagttcactggcgctttgttccagcctggacactgaccattgaaaaatagatgcctttctgtgccagcagctgctgatgcgtgccatgctccttgactctgccattctgaaacaccactattaagtctgcattctggatggtggacaggcggtgagcaatcacaatgcaggtgcggccttctctggctatgccagggcttcttggacaaccttttcacctactgtatccagagctgacgtggctcatccaaaagcaaaatantgggctgtctaacaagggcacgagctattgccatgcgtt

SEQ ID NO: 433 - DNA, 256 bp, Homo sapiens
  misc_feature (254)..(254): a, t, g, c
tttcaaaactatatatatgagatttatttcacattttctacctactcagtcatgtgagctgttgctacatttgtgaactttctgaaaccaagtgacaaatatccacaaaagatcatttacaatgtagacatcactaaagtctagatttaaaagtccagtgaaaatggcacacagttggcttacagaaataaaaaagtacaatatatttgaaatagtagggtttttgttttccatttatgcctacatcatggtgnta

SEQ ID NO: 434 - DNA, 316 bp, Homo sapiens
  misc_feature (65)..(65): a, t, g, c
gagcccgtcctgtttgggacgaccatcatggaaaacatccgctttgggaagctggaagttcgaantgaagaggtgtacacagccgcccgggaagcgaatctcacgagttcatcaccagcttccccgagggctacaacacggtcgtcggtgaacggggcactaccctgtctgggggccagaagcagcgcctggncatncgcccgagcccttatcaagcagcccacggtgctgatactggatgaagctaccagcgcgctgnatgcagagtccgagcgggttntacaggaggcctggaccgggcagtgcaggccgcacg

SEQ ID NO: 435 - DNA, 228 bp, Homo sapiens
  misc_feature (2)..(2): a, t, g, c
tnncnttttttttttttttgnggacacaacatganatnaggctttatttgaattnaaaatcttgatnccanccagggacatttttnaccgaagcgtctcagagactggcncagggnatttcttgacaagactgtacagggcttctcatcatacacaaaccctccacagcccacggctccaacccacagcacctcctgcagtccnggggggaaaagggacagtaacatg

SEQ ID NO: 436 - DNA, 455 bp, Homo sapiens
  misc_feature (16)..(16): a, t, g, c
ttcgcggcggtccgcntacgttggagggcgtgtcctgcggcgcgatggccgtagtgttgccggcggttgtggaggagctcctgagcgangatggcggcggcggtgcaggagagcgcgcgaagtacgggaccgagttcctgatgaatatctgttatcgctgaagtttctctttggctcatcagccacccaggccttggacctagttgatcgacagtccatcaccttaatctcatcacccagtggaaggcgtgtttaccagggtccttggaagttccagtaaaacatacacatgtttggcttcttgtcattactgtttcatgtcctgcattttgcattctcagtgctacgggaagagttgacagcatcctgttgcaagcatctctttgcccattttacctgagtcagtttntgagggacctttcagcagttaagtgttttttnacaagcatttnatt

SEQ ID NO: 437 - DNA, 232 bp, Homo sapiens
ttttttttttttttttcagatgtgagcaggatatttataagtgtcacaggaaaattattggatcctgcctcccaggatttctaggggatggaaagaagacagggattatggtgggaggtgattttgattggaggatttctttgagggagggaactggcagaaggagtcaggccctacggtggccctaggcagaaatccggtagttggggtggacctggggcctgacgtcgca

SEQ ID NO: 438 - DNA, 482 bp, Homo sapiens
ctaacctgttgtaagcccgcagctttgggcctggtctctgctgctcccaggcggcccctttgggtactgcctgagcaagaagtgctggagaggaggaccagtcatcaataggaggatgagattgggagagacactcggtgcaggaggctgagtgagcaggggagcactaagacccaggggtagtggaggactgctgcaacgagctggaggaggagaaggaaggcccggcgactgaagcaggcaaaggaggaggcacagatggaggtggagcaataccgcagagagcgagacacgaattccagagcaagcagcaggcggccatgggctcccaggggaacctgtctgctgaggtggagcaggctacaaggcgccaggtgcagggcatgcagagctcccagcagagaaaccgagagcgtgtcctggcccagcttcttggcatggtctgcgacgtcaggccccaggtccaccccaactaccggatt

SEQ ID NO: 439 - DNA, 361 bp, Homo sapiens
  misc_feature (4)..(4): a, t, g, c
ttgngagttgtttttgtttttaatttgtctccaataagcaatgtcttctgcccatccagcatgggaagtgggtcccacgtgaagggggcccaccacccactttgggagctgaaggagagactcgggagccctcccggctcagactccccctacgctgggtgtgggttagattcagaggcccacacccacactggggtaggttaggtatggtttacttagccacagnnccccggggnaagggagacagagtggggggcccgaagctccctgtcctgggaagagttaaaagttntgggggtagccctgggccccgtggggaagtttttaggagatgaggggaggttacttccttagcccaggg

SEQ ID NO: 440 - DNA, 433 bp, Homo sapiens
  misc_feature (59)..(59): a, t, g, c
ttataaagaaactctatatcccattgtaaatgaaaaaaagcagtatcacttgccataantagagagtaacaaaantaaacacaacangctgtacaatggtattaaagcctagctggagactattgtntcctcaagctctgggnttcctgccttccctttccgttgataaggaagcttagcaaatgttagagagacgtgaaagacacagtagtaccaaaccaaganttgntcctggggtgcagttcataganttgaaaggccactgcaaaccagtcctcttccggggtgacttgatgacttgatgggtgctcaggtgctgtgtttggggatgtgactcaaatccactnatttgtcaggcaggagtcccacactccggggnaggcccggggcccagaacggacctgnttttaggtcccgcaaggntgttcccagg

SEQ ID NO: 441 - DNA, 487 bp, Homo sapiens
  misc_feature (233)..(233): a, t, g, c
tttgcaaaaacctcatccatttattatgctctacagaaaagacacatgtttacatgattttctcgtttggatgtgaaatgcttcctgtttacaacataacagctcattgagtctttcacaggacagatgttctttatcagggcatctacaccatgcatagggccgctcttctcccgcagactcagttgcccctcagaccaggtgtggatccccacatggccattgcggcaganctctgctcctaagagagtctcagaccatcacgtgctctggtgctgaatgactgccctgtgtgacaaaacacagcctcttttttttttttttcattcaccatgtgtggcacagtttgacttcattctaatactaatactaaatggtttttactgnttcctttcaacnggtttttctttcctccagatacttaactgaggcagaaggagaagtggggtggtgggaaaaaagagtggggttgggggggtaaggangg

SEQ ID NO: 442 - DNA, 612 bp, Homo sapiens
  misc_feature (71)..(71): a, t, g, c
gagccctagaagagtgtgaaaaggaatggcaatggtgttcaccatcggcagtgcagggcagcactcattcncttgaataantgaatatttattagctggttggagagctagaacctggagaggctagaaccaagaaaggctagaacctggcaagctagaacctggagggaatgaacctggagggcntagaaccgtggagaatgagaaaaatttacatggcaaagagcccataaatcctgaccaatccnactctgaattttanagcaaaagcgtcaaaaaaaagattccctccttacccccaacccactcttntttcccaccacccactctcctctgcctcagtnagtatctggaggaagaaaacaggtgaaagaagaagtaaaaaccatttagtattaagtattagaatganagtcaaactgtgccacacatggtgaatgaaaaagnaaaaaaaagagggctgtgttttggtcacacagggcagttcattcagcaccaggagcacgtgatggtctgagactctctttaggagcagagntctgccgccaatggccatgttggggggatccacacctgggtcttgagggggcaacttgagttttgcgggnagag

SEQ ID NO: 443 - DNA, 526 bp, Homo sapiens
  misc_feature (442)..(442): a, t, g, c
agagcagtgtctaggaagtaggaccctgcctcaaatgggacttacccaggtaggcagtgagtgatatttctggaaggatcacattcccatctctctaatatgagtataacagtctcatctggatatggaaaaaagattattcagagccaacaaatctggctataggctgtttcactttctctgaaactcatccagtctgacaccagttcacaaagaacacctgtgattccagtctgatctaggcaactctagctgctttcccactaccctcaggtttgccttgtcagccatggatatgggaacagaaagactatgactcgcgcacagctctgcatgttgctgcagcttgaaggtacttgaagggttaagggagggggagggagggaggggtaatcattaaggacccttttcttttttccacccaaaagttaccttccatttntatgtggcccttcaagggttattaggggggcagcaactagttttntcctaggacacccttcgggttttgcagcntggantatgg

SEQ ID NO: 444 - DNA, 211 bp, Homo sapiens
gagaagtgtcagtttaatgaagccagcttatcagcagggcggcggagacacctgccccctcgcaggtgtgcctggctcgggctaaagtgcctgtgcagaacgaggctgcctggcggggttaggagtcggcgccctcgtcctcctcctcgggcaggatctccaggctgctgtcgggctgcggggctgtgtccgtcgagggcggcggggtggg

SEQ ID NO: 445 - DNA, 347 bp, Homo sapiens
aattcggaacgaggaggccctgcaccgccagctaaccgcctgccccgagctgccccacaacgtgcaggagatcctggggctggcccgcccgcagagccccacagcccctcgcccaccgcctccccgctgcctctgtcgcccacccgggcccgccaccccgccgccctccacggacacacctaccgcagcccgacagcagcctggagatcctgcccgacgaggaggacgagggcgccgactcctaaccccgccaggcagcctcgatctgcacaggcactttagcccgagccaggcacacctgcgagggggcaggtgtctccgccgccctgctgataagctggcttcat

SEQ ID NO: 446 - DNA, 246 bp, Homo sapiens
ttaatgccagttctcagtacataagtgcattttgaaagaggttccagctatcacttgtaaccatatatatatacatatatattctatctacaaagtgtttatttgcgagatgtttcaacggtgagttcgggccccgccgcacttgtgaccatctgtccccggtcctcgtccccgccccgcgccctggtcccgagtggcgtggggacgggccatcactgtatgtattaaaatgactgtatcaaataa

SEQ ID NO: 447 - DNA, 238 bp, Homo sapiens
  misc_feature (121)..(121): a, t, g, c
actatttaagaaacaatatttatttgcaagaaatggaaatacaaaagcataacaatttcaatttattttttttttccaaactaagtacaagtgtcctacaaagcttttttttttttttggnatttacttagctatgataaagantaaaaagtcatttaaaaaacgcgntagggncagntaacagactcacaacgtntttaggntttaaacactgctgggtctacggtaacctgttaca

SEQ ID NO: 448 - DNA, 394 bp, Homo sapiens
  misc_feature (308)..(308): a, t, g, c
acagcacctgtcccctgcaatggccctgctgtgtgatgctcatcgcttcccttcgtgctggagcagtcccccaggtgtccatctcctatctttttgttccaatcttctgtgagttccagctagcaggctttacatctggggaaaggaaaaccaggggttttagctctgttctctgctcccatccttcgctcaccagctgagtgagaacatgaactttttgcaccatgtacccatgggcttacactacttaggaaaatcaccttttcagataaaacagtttatggagttcataggaggaacaccagcantctttggacaaaactgtggagtgacccttttttaaacantgctggagcagggccctggaggntataatcaacgggtgaggntttaa

SEQ ID NO: 449 - DNA, 473 bp, Homo sapiens
  misc_feature (363)..(363): a, t, g, c
cagtttatagaatttattatgaaattgcttttcatttatacactgtaatttacttgttcatgaaagccaaaacagtggattacaatgaataacttttgaaaacaaaatggcaccccagtatttccaatgttaagataattggttgccacttattgttcggtttctttaactcttaatcttatacattctaagttactactcaaaactgccacactttaaaaactgtatgttggacattttttttttccttaataaccgaggcacaaaggatgaaaaggatttataaactatcaatgttagcataaaattacatatgactagataaatatgaaaaatatgtccatgttttctgattctaaatgnagatttctgncttcaaattgtatcagnaaccttatgngaccatctaagtaactggaacataccnaaagacctgtaaacnggattaggtggaagtagggcctattagaacc

SEQ ID NO: 450 - DNA, 690 bp, Homo sapiens
  misc_feature (650)..(650): a, t, g, c
gtcggctcttagagtagtaaccgccagaaaggagtcggaagaggtctcacgaggctgtcatcaccgccatgcccaagaataaaggtaaaggaggtaaaaacaggcgcaggggtaaaaatgagaatgaatctgaaaaaagagagttggtgtttaaagaggatggacaagagtatgctcaggtaatcaaaatgttgggaaatggacgattggaagcattgtgttttgatggtgtaaagaggttatgccatatcagagggaaattgagaaaaaaggtttggataaatacatcagacattatattggttggtctacgggactatcaggataacaaagctgatgtaattttaaagtacaatgcagatgaagctagaagcctgaaggcatatggcgagcttccagaacatgctaaaatcaatgaaacagacacatttggtcctggagatgatgatgaaatccagtttgacgatattggagatgatgatgaagacattgatgatatctaaattgaaccaagtgtttttccatgacaagtctctgaggatggttctccagttgggattttggccatcatcacccagaagagaattctttagtgtgtagttctgaaccactgattattccatgtttaagtattatccttaaactgagcnctgattcctagttatgttanccttatgtttgagtatggcc

SEQ ID NO: 451 - DNA, 277 bp, Homo sapiens
  misc_feature (240)..(240): a, t, g, c
cacaacccgagaatcagggagaaagtccctccccagcagccccctcctcctggctgggaagaatggtcccccagcaagcacttgcctgttcattcccgttcatgttttgcttctctctcagactgccttcctgcttctgggctaacctgttccagccaggctcctcatgtgacctcgcagttgagaagcccattatcgtggggcatccttttgcctacagcccctggttagggcactttngacaaggtcntgctattcagtgaacctttntacattt

SEQ ID NO: 452 - DNA, 257 bp, Homo sapiens
  misc_feature (191)..(191): a, t, g, c
gcgtgaacagagttcttattgatagtcccatcacctccaaagcagtgggcccaccctccctcctaccagcccctccccagcacccagcccctcagtgcagcgccacctcgcaggggtggaggtgggcagcacgggcagcctcgtaggcacggcggaactcctggaatcgtggggcacagccaggcccgggnttcccccaaagcggtttccagcncattgaagagggaangttgtcctgggnccgggttggnacgcca

SEQ ID NO: 453 - DNA, 303 bp, Homo sapiens
  misc_feature (8)..(8): a, t, g, c
gctttganctgcgcaaggacgggcgccccgagatagcccccgcagcccacggtctcggcgtagacggttgcctgcaagncctgcagcagacgctgagctgctcctggccgcatgcaccagcgacttcggtgagtgtccccnccatgggggagcctggagcctgccttcccctgaatgcctaccgcagccacatgcctccccacagtaattcacgngatcatccatggggtcacccatgacagtggaagctgcaggagtctgtcatcactgtggtggccgcccgtgtcntccgccaagacaccg

SEQ ID NO: 454 - DNA, 529 bp, Homo sapiens
  misc_feature (393)..(393): a, t, g, c
ttttttttttttttttttcccagtttcatggaacttttattgagtttatttgtgggatgcatgacaggaggtctttccatcattagtaagaatgaaaggtcattttcacagtcactttggcacacgctaacgtctcataaaaaaaaccagaaaagcaaagacaatggaacctatagaatacgtcattaaatacatacaaaacactaataaaatatccctgataaaccaaagtgcatatgcccaggacagtattgcaccttccccagtcgcgcgtgtcgctcgcatggcctcgtcaaagttggaagttaacagtcgtgagattagtacgcaggtgcacaccagttatttacagaacggcggtcagagccgcgggagtaggggccggccgcccgnagggtgcggtgtgaacagaggcggtggctggggtggangggcggggaggagtgctacatggatcgcggctcagggccagggggtcgcccctgggtgtaaagccgcctgttgaacancctcngggctccatgcaaca

SEQ ID NO: 455 - DNA, 596 bp, Homo sapiens
  misc_feature (502)..(502): a, t, g, c
cttaaagttgaaaggtaacgggattggctgccaactccagcttcagagggggaacgtgggccagtagaaggcagggaaggtgtaggggagccggtgcgttcatccaccttttgccaaggttgaggtttccaagtgcagacggcttctctccttgggctgagcggtgcgcaccgtccgtctccgcggactggagtctgccctgatgccccactggccagtcggcgctgactgttgcatggagcccccgagggtgctccacagcgcggccttacacccagggggcgaccccctgggccctgagccgcgatccatgtagcactcctccccgcccctccaccccagccaccgcctctgttcacaccgcacccctgccgggcggccggccctactcccgcggctctgaccgccgttctgtaaataactggtgtgcactgcgtactaatctcacgactgttaattccaatttgacgaggcatcgagcgacacggcgactggggaagtnaatactgtcctggatatnacttggttanaaggatatttatagtgtttgaagtattaatangtaattaaaaggtccatgcttggttctggttttt

SEQ ID NO: 456 - DNA, 446 bp, Homo sapiens
  misc_feature (1)..(1): a, t, g, c
nannngatttttattatatgaagatggtatacaaaatacattcatcatgactagaaatataggaccaaaccatgtctgtcttatatctgtagcatatatncttggtttgtataaaagtaactttaaaattccagtttccttaaatagttatgcacaaaacacacatacacccacacacacacacacacacacacacacacatacagttacaccactgtcgggccaaagatgcactcctcctttaatcaatttaaatgagggctaggcgaggtatctgtttgatgtttgcattcttgtgggggctagggaaacaagggcacggggtccctaaaattaacatctcgggtgttcacttcttggggactggacagggncacaggacttggcactgggggtttcaggcccctttccnccccggactgtttccccggtacatttttntcggg

SEQ ID NO: 457 - DNA, 463 bp, Homo sapiens
ggagtcttaatgtagaaagaaaaatggagacttgtaataatgagctagttacaaagtgcttgttcattaaaatagcactgaaaattgaaacatgaattaactgataatattccaatcatttgccatttatgacaaaaatggttggcactaacaaagaacgagcacttcctttcagagtttctgagataatgtacgtggaacagtctgggtggaatggggctgaaaccatgtgcaagtctgtgtcttgtcagtccaagaagtgacaccgagatgttaattttagggacccgtgccttgtttcctagcccacaagaatgcaaacatcaaacagatacttcgctagcctcatttaaattgatttaaaggagggagtggcatctttgggccgacagtgggtgttaactgttatggtgtgtgttgtgtgtgtgtgtgtgtgtgtggggtgtatgtgtgttttgtgcct

SEQ ID NO: 458 - DNA, 400 bp, Homo sapiens
  misc_feature (174)..(174): a, t, g, c
gaagacatatttcagttttattatcttttaggcatagacgggtatgttaatccatttccacaaacaattcctataatcacatatagggagtgaaccttgaacttgcaaaacctgtttcctgttgccaaagagttaaattggagagccgaaacctcaaaagttgaggatttgganttctttttcttcttaggcatcttcgtgcatgtgggctgccattaaacaagtaagctttcctctttatccggggactggaatcgatgggtaataatgttgnctttttttttcccgggcaaactttctgctttcgggtccagagctctgagtttctcatgttctgctctcgagggttctgacagctgtttttgggactttaatttaaccattgcagtgggggaaccag

SEQ ID NO: 459 - DNA, 408 bp, Homo sapiens
  misc_feature (405)..(405): a, t, g, c
ataaaagcttccgtagtgcattggtatggattaaatgcataaaatattcttagactcgatgctgtataaaatattatgggaaaaaaagaaaatacgttattttgcctctaaacttttattgaagttttatttggcaggaaaaaaaattgaatcttggtcaacatttaaaccaaagtaaaaggggaaaaaccaaagttatttgttttgcatggctaagccattctgttatctctgtaaatactgtgatttcttttttattttctctttagaattttgttaaagaaattctaaaatttttaaacacctgctctccacaataaatcacaaacactaaaataaaattacttccatatgaaatattattttctcttttgggtgtggggagatccaaagggtttaaagtcntaa

SEQ ID NO: 460 - DNA, 453 bp, Homo sapiens
tttttttttttttttttttgaggttttgctatgctgaatactttatttatatttttatgtaaacataatttgctttataggcacaatataacaaaagctctaaagccacaaagacattagacaatgtttcagttcacttcaatcaagcttaaattacagagaagccttcacagttcctttaaggcagactgagacattctttacacttccttcagaaaaaacaatcaagagacaggaaagaaggtgcttaaaatgagagtacgttaaacaattaacttgcgtcccccggttcttcatggcagaaattggtcttattggtcaaactctagttttctcgttctgtattctgaaagggttttctttaaccatctggagtaatttgcgattactggattaactttgctagttaaataaaaccaaaccaaacccagatttggtaggggtggtggggga

SEQ ID NO: 461 - DNA, 359 bp, Homo sapiens
tttaagtatattttcctttattgttttcaggtacttactgaacattaccaatagtctttcattactttggattaatttatagataggtattaataccaataatagtaattacaggctatcatttttttgcacacatcttaaatcctgaaacaagcttgagtttcttctccgttcttcagacatcagcagaaagaggttccttcttaatggaaaatctaattcatcttatcttctgagtcctccgtgcctggagtagtacttggcttgtggcaataaactgtttctgctcggcagtcctggcttcaagcctcccacaggcagattctttttaatgctccaaatcccttccacggccactg

SEQ ID NO: 462 - DNA, 342 bp, Homo sapiens
tttcattaaaaatccagctgcagtggccgttgaagggatttggagcattaaaaagaatctgcctgtgggaggcttgaagccaggactgccgagcagaaacagtttattgccacaagccaagtactactccaggcacggaggactcagaagataagatgaattagattttccattaagaaggaacctctttctgctgatgtctgaagaacggagaagaaactcaagcttgtttcaggatttaagatgtgtgcaaaaaaatgatagcctgtaattactattattggtattaatacctatctataaattaatccaaagtaatgaaagactattggtaatgttcag

SEQ ID NO: 463 - DNA, 670 bp, Homo sapiens
ttttttcttttccttttttatttgtcatagtccttccttcatctcactccaactcttctttcctggctccccgctcggctccaccgccgcgggcctcccttcacacatagcacaggtagaggtacgtcttctctatcctccagtcgggcgggatctcctcgggcttgtggcccttcatgtgcttctgcatggaggagaggctggggcagtactctgtgcaaatggtgcactggtagggcgaggcgccgttgtgcgttcttaggtgcttgatcatggccgagtagtcccgggagcgctggtggcagagcttacactcaaagggcttctcacctgtgtgcaccctatagtgcgtctccagctgatgcttgaggctgaacttcttgccacagccattgcactcgtagggtttctcacccgtgtggatgcgtttgtggctcttgagtgtgctctcatcccggaagcagctgccacagaactcacactcgtaggggtggtcgcctgtatgtgagcgcaggtggcgtttgagagccgtgtggctggggaaggggcggttgcactcactgcagatgtagctgcgcacgcccgcgtggacctccatgtgctgctgcagtgcgctctgcgcctggaaacgcttcccacacagcagacagaagacggccatgtcagtgcc

SEQ ID NO: 464 - DNA, 320 bp, Homo sapiens
cacagaaaaaaaaaatttaatattcaacatgcaaaacaacctttaaaagaaacatgaaatcataaagcaaagctaacagccaaccaacaaataccgcctagcaatgatttccactggatgtggaagagggttaataaagacgctgttggtaacgcgtacagaactatcactggcaatcagcatactgagctatccagtggaggccggcgccgtgtttttgctaaaatacatgttgtagaagtcataattcatagtgaagaatctcaacaggttttcttacagatttaattactctcacacaaataattcatttggaaacc

SEQ ID NO: 465 - DNA, 364 bp, Homo sapiens
cttgacagcccactggttcttcctgtcctcttgctctagtctgtatcagaaagcagaatgactgtacttttgttttacaaacaaccacctgataggacggacactccacgagataaggaaaggcacgtgcccttgagcttgaatggaagcagcctctggagggggcagccactgccgttcgagggagagcagctcttcagcagtggccagagtgccacgtgactctgcagatgacccctgggagccgggtgatgggcacctgctggggcttttgttttttctttttcactggctggcttgatcctcagtggcaaaaggacccctgagccccttctccgagccctggagcactcctcgggacacc

SEQ ID NO: 466 - DNA, 508 bp, Homo sapiens
taggattcgatgcccaccaaaattcttgtaaatgaagcaggacttgatttcaaattctgtctatacacacaatatttggccacatagaccactccccaaagtctgcaaaacactgcctactgggcaggcttacagtgacagaaaagtatgagaacacaatatattatttttataaagactaaaatcagatttaggctgtctagatatcttattccagaaaacacagatttaagatttttcagtgattcttgccttccccctccccttttcttccccaatgagataaccatttctttcacaatgatgaaccatccctttttatggaaaaatggctttctttctccattggatcaggacaaagacatcacttcttggccacactggccacagccttcttggctgcatcctccaggtcaatggctgaagtaatggggagtccgctgttgttgagtatcttctgggcctcttggacgttgggtccctcaagccggaccaccagggggacctt

SEQ ID NO: 467 - DNA, 323 bp, Homo sapiens
  misc_feature (129)..(129): a, t, g, c
gctcattgcatgatttcatctttaattaaataagtcaagtaggccatcaggagttgggttcacattctattctgcaaagcaggccctgactgctggggctgccagctctctgaagggcagccccacgtnttccaggagacggcagattgtctgtggccatggcaaggacatctgggacgacagtgacagcaatggggctgggagcaatgtggaacggcgccagagntgaagcagaatgagctacatgctacacatacacacacgtggatgcatatgtacataccatggcagtgtatnccgtnaaggggtntctcgtaccatgc

SEQ ID NO: 468 - DNA, 574 bp, Homo sapiens
  misc_feature (480)..(480): a, t, g, c
cgtgtcctttggcaccccgtaactggactggggacaaatttgttacgtgtttccaaggctacagacatggcgccatcctcacaggcctagctcatgtcatggatcaggtgctttgctggggggatacaatgacccatgtggtctgcgtcttctgccattggcttgaactgggcaccccctgaagtctccatctcagctgtatattctagtccaaattttcctggctcccctgctccctcccaggtcctgcacactgtctttttgccaacacctcagccctgtttgctatttcatgtctgcatggtacgagacaccccttcacggcatacactgccatggtatgtacatatgcatccacgtgtgtgtatgtgtagcatgtagctcattctgctcagcctctggcgccgttccacattgctccagccccattgctgtcactgtcgtcccagatgtccttgccatggccacagacaatctgcngtctcctggaaacgctggggtgccttcagagagtggcagccccagnagtcagggctgctttgagaatngaatgtgaaaccaantcctatngcct

SEQ ID NO: 469 - DNA, 417 bp, Homo sapiens
  misc_feature (114)..(114): a, t, g, c
tttttgtcagagtataaaaaagtgagtgcagcaaaacaaggaagacgagacaggcgaccacctcgtgaacgaagattcgaaaagccacttgaagaaaagggtgaaggaggcganttttcagttgatagaccgnttattgaccgacctattcgaggtcgtggtggtcttggaagaggtcgagggggccgtggacgtggaatgggccgaggagatggatttgattctcgtggcaaacgtgaatttgataggcatagtggaagtgatagatctggcctgaagcacgaggacaaacgtggaggtagcggntctcacaactggggaactttcaaagacganttaactgacttggatcantcaaatgttgactgaagtacaggtcgtgcagaaaaccntcgtgccgattttttggcctcgagg

SEQ ID NO: 470 - DNA, 447 bp, Homo sapiens
  misc_feature (369)..(369): a, t, g, c
gatacaatttacagctatggtaagatgattaccacagccctgattcaggtgtggtctttagcttcagaaagtccatcggtcatgtagctgactgtcattgctaaatctgttgcttgcagaggcctcagtgctctggctttgcgagtagagtcctcaccatttaagctcttcctacatacaggacatgtgtcatgcagttctagccacggcacaatacaactgctgtgaaagaagtgattgcaaggtaactgccggacttcctcttcaactgtgtaatcttctttgcatactggacactctaaacccatatcaacttgttcctgagttactgtcactgttggaagagatgtgatcttttccttgtcagcngggggagggcctgtgttttccagttgtcctaaaagctgggttacaatggcatcaagccctgtctgaccccaggcatag

SEQ ID NO: 471 - DNA, 318 bp, Homo sapiens
  misc_feature (127)..(127): a, t, g, c
aatagagccaatgaaaggggtcaccagactcacactgacttctggggagcaagacctccacggttgccattgggtcggagatacagatctcgaggaagttctcgtcctgacagatctccagctattnaaggaatactacaacacatctttgcaggattctttgcaaattctgccattcctggatctccacaccctttttcctggagcgggatgctgcactccaaccctggggactatgcctggggtcagacagggcttgatgccatttgtaacccaagccttttatggacaactggaaaacacaaggccctcccccaa

SEQ ID NO: 472 - DNA, 502 bp, Homo sapiens
  misc_feature (30)..(30): a, t, g, c
tttttttttgtgtgtgcttatttgggaganggattactgcatttattctccagaaaaaaaggcagacctgaggcatcacanttacccggaacatttaaatatgatgacatgtttcttatcttctaaacttcaacgaaagctgcttgtacaaactatttaatgcatatctgccccattaaacaagatatttgatgtagcagagaagccagaagctgagtcacttcatttctttctgttgcctccagttgccagcatagttgcaactcgcataaatatatttaatgtatccatgtaggatactcagcatcgagttaatggggatcatatttttgaactcccatacattgggnggatacttctgcacggcttgattacctttctgggggtatcatacaggagggnaccatgctggaaaaggnacttaatccacccgtaccatttgcccactgaggtaaagngtgggcaccnggccgggnttggntagggtgggagggacctaggt

SEQ ID NO: 473 - DNA, 212 bp, Homo sapiens
  misc_feature (33)..(33): a, t, g, c
tctttgaggccgacgctaggggccaggaagganacctgcgaggcgaaggtgaccggggaccacgcatttcagatctgctcggtagacctggtgcaccaccaccatgttggctgcaaggctggtntgtntccggacaataccttctagggttttccacccagctttnaccaaggnctcccctgttgtgaagaattccatcacgaagaatcaat

SEQ ID NO: 474 - DNA, 480 bp, Homo sapiens
cagagactttgaagtctgctacccagaggagcctctcagggactggccggagatctccctgctgaccgagaacgaccgccactaccacattccagtcttttaagtccgctgggggccgaacagcagtgctcaccagtgacggtggtcacagttgcaataaagtctctctctgaaaccaaagctagcatttcagcatggaaggaattaggaccttttcttcaggattacaggtacactggatgcagccatcatggatggtttttctttatttttcagtgatttcctctgaagcagctgcactgatacatttgggagttggtggcttgactttgtccataaggggcgtgccacttcacatgatggcgggcctttaagagcacaaagaagtttaatatggacaacaacaggaaaaagcaagaagaaaacaagtagggaaaaacagctaacctggagagaaagaatttctttaacctttatgtt

SEQ ID NO: 475 - DNA, 201 bp, Homo sapiens
cggatcacaacttttactgcagccgctattttattgccaatcagccatgagccccgccttccatacacaatgacatttcatccccacaatcgattaacacaaccatgatagccatgaactcccaactcctccagctgctagtgctcaacgggagagtcccctccaggtctgtctcattgcagagcccatattctttctgcc

SEQ ID NO: 476 - DNA, 494 bp, Homo sapiens
  misc_feature (428)..(428): a, t, g, c
acatttgcctcggtttttattgagtggatctctcacgacaaaatcatgaatattacactgaaaggcttattacattatctttgtgtagttactctccagtataaaccctgtgatgttccggttttgatgcctgggtaaaagcttaagcatgcacgttacatttcgtatggtttcatcaaaaaagtttttgatgcctagtgagactttggcctgcggaaaatctctatcacatataattattataaatgctctttagtatggattctctgatgttgatgaatgtttgaagtcataatggtttcccactctcagtgtttttgtttctctcaagcatgaatttttgcaatattgtacaatgtgagaattgtgccagaagaccttgccacattcattacatttgttaaggtttctcaccagcaagaattccnttgaagaatccnggtctcagattttaccttaagaccctgccacattcagtacattaggaaaagttt

SEQ ID NO: 477 - DNA, 312 bp, Homo sapiens
  misc_feature (236)..(236): a, t, g, c
ggcacgagggagatttcatgccggaaagaaatctaacacatgtaattaatgtggcagagtgttcagttagcattaaagccttgtaagacatacaataatttatactggagaaaaactttgcaagtataatgaatgtagcagacctttagtttttgttcaaggcttaataaccgttagctagaccatagaggacagaaactttactaatgtactgaatgtggcaaggtcttaaggtnaaatctgagaccaggattcttcngaacgaattcttgcttggtgncggaacctaacaaatgtnatgaattgtggcaa

SEQ ID NO: 478 - DNA, 252 bp, Homo sapiens
  misc_feature (236)..(236): a, t, g, c
catagttcatagtttattacaaatgcatattatccaactcagtagaaatccatcgtaccccagaatgtacagaaggtatgcaatgttccagagtgtcattgtcagctctggctttacatatatattaaatatatatatgttttgagacagggtctcgctgtcacccaggctggggcatcattctaagagccagaacacagaataacacctgataaaggtagcaattccagaataanccaggtctagntcatc

SEQ ID NO: 479 - DNA, 525 bp, Homo sapiens
  misc_feature (306)..(306): a, t, g, c
tttttttttttggtggggaggaaatcttttttatttttatacatgacaagattttacatcaagaatagtcagttaaatagtacaaatttacattcatgaggtatgtttaaaaaaaatcaactaaaaaacccacttcttattgtaacccataatcccacattttacagtgcaggggagaaggggactggtggggggcatccaaaacaagtctctgccaaaagaaatgacttacatttcacattccctctccacacaggatccaaacgatgagagtataatttacaattcatctttttcagctgtagnttcctttgctgtttcttgtttttcttcatctgttcccacatccatttctttgtctttgtcttgctctttgtcttctgctgtctcctcagggtcttcggcggggttcttccncctttgcatcaggggtntgttcaaactgagggaagggattctttctattcnatctccctatggctttagngtctgggaaaaggcatcccnggcccgnttcactggtgg

SEQ ID NO: 480 - DNA, 384 bp, Homo sapiens
  misc_feature (17)..(17): a, t, g, c
cagcaagactgaaactnttaaggagccatgaaggaagaagcagcagcaaaagaagagaaagaagaatcttatgatgaagctgcagtagaggaagaagaagaaaaacagaaaccaaagactaaaaaagttgaaaaaactgtctgtgaatgggaacttatgaatgatatcaaaccaatatggcagagaccatccaagaagtagaagatgaatacaaagctttctgcaaatcattttcaaaggaaagtgatgaccctgtggcttgtattcactttactgctggaaggagaagttaccttcaaattcaattttatttgtacccacatttgttccacgtgggcctatttgatggatttgggatcttaaaaagngccggttacatttagg

SEQ ID NO: 481 - DNA, 339 bp, Homo sapiens
ttttatattcttcctttattggctgtccttgtataatacaaatcttcaagtttagatacaaaaaaaatctggaaataaaagatagaaaagtacccgccaggcaccccttccccttcttcccggacccctccccagcgggcaccgacatgaggtaactgcgcatcttctcctcctcctccctccccgcctcctgggcccccaacttggccctagcccccatgagaccttggcccaaggaagacatgggccccaaagttggagggtgatggaggtgagtacagcattggcagtgaacgcagcgtgtgggcagagggcagcccttagcttgtgctgtgcatg

SEQ ID NO: 482 - DNA, 451 bp, Homo sapiens
tttttatttttatttctttttatttaatgatgcaaaatacaaatgctggcttgatatatattcacatataactttttctctcacatttacaactcaacagatggtgaatccagaggaacagctacaatcactatacggagcatactacattagtgtttcaaaagtttattccagcccatttcttcttttgtactctaaaatatcttcattgtgttgtttgatgagatagatccacaagaagacagtggtgcccaaggtgaacccaactttcagccagtcaggtttggcattcggcggctctcgcacgtagaactttgatcgcactgaagggccgctcgggagcctggcgggggccagcagccgggaaaggggacgcagcaaggcggacggcgccatcttgcgtggcccagctcagtctctccgagttggcaacagaaccagcgccacctgg

SEQ ID NO: 483 - DNA, 432 bp, Homo sapiens
  misc_feature (260)..(260): a, t, g, c
tgccagacttttagaaattaagtagagcacattttaaataaagtggtattgctttcatctttaccccatttttgctgaatgcttgtataagagtttaatacttgggcacttaggaccactcttcatgttctataactaaaaggaccagtgtatttgtaaactaactggggcatggcagtcaacccagatttgcagcaaatcttcacccatattgcaaaccagatggagaacttaactattttatggtatctagcagctgncttctttttgtgntcacatgcatagagctaaacccacaggctgctgtgtacccctatgaagtaagaggcttagnaatcatatttntccaggttttcccctggntcacttctaaaatactcttgaaacatttaaccaatttgaggggaaaccnaataagcgancaccaggaag

SEQ ID NO: 484 - DNA, 413 bp, Homo sapiens
agcatttaattctgattttaatttccataaaattaactctacaaagatttctcaaagaaatctgaccgccttcaaactacatgtttgttttgtattacatgtaatagatttccacatatgaaaaaatcataaaatgaaggctacgttttccaaccgctatttaagtactgtttagaaactatcgttgaatgaatgaacctgattttgcctcatcgtgtttatctaaaagagtctttgaaagatatttattttccattttctttacacggtccctctttttcaatctgtaaataaaaattaatgagcaatttaggacacaatatcacaacacaattattctgtaattgatctgataaccaaagacacataagaataggatgatctataggatagatgtttccacacagcatttg

SEQ ID NO: 485 - DNA, 196 bp, Homo sapiens
  misc_feature (184)..(184): a, t, g, c
cctgtttgggcccatttattgtttattttgaataggtaatacagtcacatggttcaaaattcaaaatgtacaaaagggtgtaagtgttaagacccttccctctcctgtcctcaacctactggttccccctccccaaaagtaatcagtgttacccatttattctgaatagggcatacattaagcnattgatgtgttt

SEQ ID NO: 486 - DNA, 465 bp, Homo sapiens
  misc_feature (433)..(433): a, t, g, c
gcaacagctcgtttgggaggctggagtggaaacaaagggtgggcatcaaagatgagaagccaaagcccctacaactccagccacccagccaggaggggctgtccaatcacattcaggcatgcgaatgagctgggccctgggtgaggtgggggtctggcctagtggggagcggcctggcctgggtggggcagggctggcctggtccagctgtgggctccattcccatcactgctgtccctcctgaggtctggattggggatggggacaaagaaatagcaagagatgagaaacaacagaaacttttttctctaaaggactggttaaatcaattctgatacagccttacaatacaatagtattgcagctaaaaaataattgtatggctttatatactaatatgtaataatctttcagggtgaaaaagggcaagccnccagaaatgttgtatangcgcacttcccattg

(19) United States
(12) Patent Application Publication
(10) Pub. No.: US 2010/0131286 A1
(43) Pub. Date: May 27, 2010

(54) METHODS FOR THE PROGNOSIS OR FOR THE DIAGNOSIS OF A THYROID DISEASE

(75) Inventors: Remi Houlgatte, Bourgneuf En Retz (FR); Brigitte Franc Nee Maleville, Paris (FR); Jean-Fred Fontaine, Angers (FR); Yves Malthiery, Anger (FR); Frederique Savagner, Saint Sylvain (FR)

Correspondence Address: YOUNG & THOMPSON, 209 Madison Street, Suite 500, Alexandria, VA, 22314, US

(73) Assignees: UNIVERSITE D'ANGERS, Paris (FR); ASSISTANCE PUBLIQUE HOPITAUX DE PARIS, Paris (FR)

(21) Appl. No.: 12/593,525

(22) PCT Filed: Mar. 28, 2008
(86) PCT No.: PCT/EP08/53773

§ 371 Date: Sep. 28, 2009

(30) Foreign Application Priority Data
Mar. 29, 2007 (EP) 07290373.5

(51) Int. Cl. C12Q 1/68; G06Q 50/00

(52) U.S. Cl. 705/2; 435/6

(57) ABSTRACT

The present invention relates to methods for predicting or diagnosing a thyroid disease in an individual.

### FIELD OF THE INVENTION

The present invention relates to methods for predicting or diagnosing a specific thyroid disease in an individual, which methods are based on the quantification of the level of expression of thyroid disease-specific marker genes.

### BACKGROUND OF THE INVENTION

Thyroid is a small endocrine gland that mainly produces two thyroid hormones, respectively tri-iodothyronine (T3) and thyroxine (T4). Thyroid is the subject of various dysfunctions that affect a large number of individuals worldwide, with diverse health consequences, including death. Six classes of differentiated thyroid diseases originating from follicular cells have, to date, been determined which are, respectively (i) follicular adenoma and carcinoma, (ii) papillary carcinoma, (iii) oncocytic adenoma and carcinoma, (iv) Grave's disease, (v) autoimmune thyroiditis and (vi) atypical adenomas.

To date, clinical classification of thyroid diseases, as established by the World Heath Organization (WHO), is mainly based, if not exclusively based, on conventional parameters such as hormone dosage and histology methods. Regarding more particularly thyroid tumors, the one skilled in the art may refer to the WHO classification (DeLellis R A, Lloyd R V, Heitz P U, Eng C (eds) (2004) World Health Organization classification of tumours: Tumours of endocrine organs. IARC, Lyons).

However, this conventional classification of thyroid tumors does not allow a systematic and clear distinction between different thyroid tumors. Illustratively, some thyroid tumors, such as adenomas, are histologically highly heterogeneous, presenting a trabecular, micro- or macrofollicular appearance. The classical features distinguishing carcinomas from adenomas, such as vascular or capsular invasion, are sometimes difficult to evaluate and can lead to interobserver variations (Hirokawa et al., 2002, Am J Surg Pathol, Vol. 26: 1508-1514; Franc et al., 2003, Hum Pathol, Vol. 34: 1092-1100; Lloyd et al., 2004, Am J Surg Pathol, Vol. 28: 1336-13340). Occasionally, adenomas without vascular or capsular invasion show pronounced cellular proliferation, irregular cytological characteristics or unusual histological patterns leading to their classification as atypical adenomas. According to the WHO classification, this term does not correspond to any specific biological behaviour (DeLellis et al., 2004, World Health Organization Classification of Tumours: Pathology and Genetics of Tumours of Endocrine Organs. In: 320).

The annual incidence of thyroid cancer varies considerably in different registries, ranging from 1.2 to 2.6 per 100,000 individuals in men and from 2.0 to 3.8 per 100,000 individuals in women. It is particularly elevated in Iceland and Hawaii, being nearly two times higher than in North European countries, Canada and the USA. Recorded incidence of thyroid cancer increased by 2.4 fold between 1973 and 2002. However, this increase has been attributed to increased diagnosis of tumors measuring less than 2 cm in diameter, and has not been associated with an increase in mortality.

The above clinical data illustrate that there is a need in the art for an improved prognosis or diagnosis of thyroid diseases, including thyroid tumors, in view of starting a suitable medical treatment as early as possible, so as to improve the clinical outcome.

Attempts have been made in the art for identifying new markers for improving diagnosis of several thyroid cancers, namely papillary thyroid carcinoma, follicular thyroid carcinomas and thyroid oncocytic tumors, through gene or protein profiling techniques.

Immunohistochemical analysis does not accurately differentiate between benign and malignant lesions. This is particularly true in the case of follicular lesions. Indeed, many of the markers proposed have failed to produce convincing results. Galectin-3 seemed to be the best marker to distinguish between follicular benign and malignant lesions (Hermann M, 2002, Arch Pathol Lab Med, Vol. 126(no 6): 710-716). However, this marker is less informative when dealing with controversial classes of tumor, such as oncocytic adenoma or carcinoma (Nascimento M, 2001, Endocr Pathol, Vol. 12(no 3): 275-279; Oestreicher-Kedem Y, 2004, Head neck, Vol. 26(no 11): 960-966).

In this context, marker genes that are either under-expressed or over-expressed in papillary thyroid carcinomas have already been identified (Huang et al., 2001, Proc. Natl. Acad Sci, Vol. 98(no 26): 15044-15049; Yano et al., 2004, Clinical Cancer Research, Vol. 10: 2035-2043; Baris et al., 2005, Oncogene, Vol. 24: 4155-4161; Jarzab et al., 2005, Cancer Research, Vol. 65(no 4): 1587-1597).

However, most gene profiling studies known in the art, including those cited above, have compared carcinomas and benign lesions in only a few types of tumors.

There have been two studies on a larger number of different tumors, one distinguishing between benign and malignant tumors (Finlay et al., 2004, The Journal of Clinical Endocrinology & Metabolism, Vol. 89(no 7): 3214-3223), and the other focussing on follicular thyroid carcinomas and the expression of the PAX8-PPARγ fusion protein (Giordano et al., 2006, Clinical Cancer Research, Vol. 12: 1983-1993). However, since none of these studies had analyzed a wide range of types of thyroid tumors, including atypical cases, the candidate gene markers proposed cannot be considered specific.

Two microarray studies have proposed combination of genes to distinguish between FTC and benign lesions or PTC (Weber et al., 2005, J Clin Endocrinol Metab, Vol. 90: 2512-2521). While these small sets of genes may help predict the most frequent lesions of the thyroid, they give no information on, notably, atypical nodules.

Although some attempts have been made in the art for defining new markers of thyroid cancers, it appears that further studies are needed for validating their specificity.

Beyond the strict requirement for a high statistical relevance and a high specificity of any novel marker for a thyroid disease, including a thyroid cancer, there is also a need in the art for a method that would allow an exhaustive prognosis or diagnosis of any one of the known thyroid disease, and not solely a diagnosis of a single thyroid pathology or few thyroid pathologies.

### SUMMARY OF THE INVENTION

The present invention relates to an integrated in vitro method for predicting or diagnosing a specific thyroid disease in an individual, which method is based on the quantification of the expression level of disease-specific marker genes, selected from the group consisting of marker genes specific for (i) autoimmune thyroiditis (AT), (ii) marker genes specific for Grave's disease (GD), (iii) marker genes specific for macrofollicular adenoma (FTA-a), (iv) marker genes specific for atypical follicular adenoma (FTA-aty), (v) marker genes specific for microfollicular adenoma (FTA-b), (vi) marker genes specific for follicular carcinoma (FTC), (vii) marker genes specific for multinodular goitres (MNG), (viii) marker genes specific for oncocytic adenoma (OTA), (ix) marker genes specific for atypical oncocytic adenoma (OTA-aty), (x) marker genes specific for oncocytic carcinoma (OTC) and (xi) marker genes specific for papillary carcinoma (PTC).

### BRIEF DESCRIPTION OF THE FIGURES

FIG. 1: Gene selection by the average separation parameter.

This graph shows all possible sets of best genes on the X-axis, i.e. with the lowest P-values, and the corresponding average separation parameters on the Y-axis. Low values of separation indicate well-separated classes and high values poorly-separated classes. The parameter has to be minimized while the gene selection size has to be maximized to improve the power of analysis. A point of discontinuity represents the optimal average separation equal to 0.1732 for the 1197 best ranked genes.

FIG. 2: Effect of sample filtering on data set homogeneity.

This figure shows homogeneity values for each tissue from the original data set (grey bars) and from the filtered data set (black bars). The average homogeneity (Hₐᵥₑ) of the original data set is equal to 0.486. The average homogeneity of the filtered data set is equal to 0.515.

### DETAILED DESCRIPTION OF THE INVENTION

The present inventors have simultaneously analyzed the gene-expression signatures of more than 90% of the medically-recognized thyroid lesions in order to select disease-specific marker genes for each class of thyroid disease, and especially of each class of thyroid tumor. All differentiated follicular tumors or pathologies, as well as the wild type tissue (WT), were represented in the analysis: macrofollicular (FTA-a) and microfollicular (FTA-b) adenomas, multinodular goitres (MNG), follicular (FTC) and papillary (PTC) carcinomas, autoimmune thyroiditis (AT) and Graves' disease (GD). The analysis performed by the inventors also includes atypical adenomas and oncocytic tumors associated to follicular or papillary tumors: atypical follicular adenomas (FTA-aty), oncocytic adenomas (OTA), atypical oncocytic adenomas (OTA-aty) and oncocytic carcinomas (OTC). The molecular classification of the tumors has then been compared to the pathologic diagnostic groups.

The experimental work performed by the inventors has allowed the identification of disease-specific marker genes, which expression is deregulated, specifically in patients suffering from a given thyroid disease selected from the group of marker genes consisting of (i) marker genes specific for autoimmune thyroiditis (AT), (ii) marker genes specific for Grave's disease (GD), (iii) marker genes specific for macrofollicular adenoma (FTA-a), (iv) marker genes specific for atypical follicular adenoma (FTA-aty), (v) marker genes specific for microfollicular adenoma (FTA-b), (vi) marker genes specific for follicular carcinoma (FTC), (vii) marker genes specific for multinodular goitres (MNG), (viii) marker genes specific for oncocytic adenoma (OTA), (ix) marker genes specific for atypical oncocytic adenoma (OTA-aty), (x) marker genes specific for oncocytic carcinoma (OTC) and (xi) marker genes specific for papillary carcinoma (PTC).

Thus, the present invention provides for an in vitro method allowing (1) detecting a deregulation of the expression of one or more disease-specific marker genes, each marker gene being comprised in a single group of marker genes among groups (i) to (xi) listed above, and then (2) predicting or diagnosing the occurrence of a specific kind of thyroid disease, the said specific kind of thyroid disease being determined by determining which group (i) to (xi) comprises the marker gene(s) for which a deregulation has been detected.

An object of the present invention consists of an integrated in vitro method for the prediction or for the diagnosis of a specific thyroid disease in a patient comprising the steps of:
a) providing a thyroid tissue sample previously collected from the said patient;
b) quantifying, in the said thyroid tissue sample, the expression level of two or more disease-specific marker genes that are indicative of the risk of occurrence of, or of the occurrence of, a specific thyroid disease, wherein the said two or more disease-specific marker genes are selected from two or more of the groups of marker genes consisting of:
   (i) marker genes specific for autoimmune thyroiditis (AT) that are contained as inserted DNA in the recombinant vectors selected from the group of vectors having the following IMAGE clone ID No:
   (i-1) 669510, 725096, 366100, 325155, 154172, 787938, 744055, 592276, 740941, 73778, 667174, 739193, 295412, 23827, 136686, 744385, 471641, 724276, 28584, 268972, 739097, 744943, 724609, 2074228, 365913, 687782, 23872, 668452, 681959, 687379, 23271, 731745, 738332, 726760, 666911, 724150, 682555, 587186, 813260, 136646, 309645, 809828, 487152, 564994, 667348, 730149, 731115, 738945, 985457, 179288, 742862, 78353, 503189, 1087348, 753215, 471574, 471696, 549054, 744657, 731357, 713422, 486708, 666794, 726830, 730829, 300268, 725364, 665376, 375746, 324079, 365271, 172982, 724562, 839101, 265558, 52226, 713922, 136919, 682119, 745106, 1877668, 1594414, 471829, 739956, 23945, 173841, 31120, 342181, 742580, 135941, 32339, 687912, 666154, 743774, 738970, 22711, 501971, 364846, 175864, 666792, 724416, 174683, 667892, 744087, 68950, 2467442, 136807, 744439, 1716286, 121551, 744797, 742061, 666778, 740158, 724596, 143759, 666315, 50202, 667514, 364959, 258666, 148796, 364324, 72744, 669263, 667533, 365177, 2055272, 731298, 512910, 470-408, 137720, 2237263, 712295, 284828, 81911, 251452, 135705, 153667, 666007, 2266583, 136863, 724119, 136223, 727251, 485872, 137238, 713653, 364412, 471702, 668684, 727056, 665674, 251250, 364706, 136260, 136560, 738938, 382423, 430263, 2139164, 309697, 773192, 726272, 682088, 666367, 21716, 135689, 383945, 727289, 724554, 364729, 665649, 668152, 811582, 666551, 472111, 489945, 486304, 416744, 258865, 744050, 743016, 1846326, 509606, 666110, 51232, 3659591, 667355, 364974, 666860, 179266, 727092, 24176, 725076, 137353, 307094, 23765, 666168, 725836, 172996, 364926, 525221, 472160, 667110, 730012, 135980, 136014, 2046361, 726763, 731726, 135887, 365288, 44881, 724508, 146976, 744616, 366159, 724895, 724892, 668912, 743987, 23817, 665538, 687625, 136606, 713879, 26164, 667587, 731410, 743422, 687532, 726335, 669318, 214611, 669181, 713647, 178792, 1417901, 135766, 364469, 565317, 382738, 669188, 666361, 667117, 591907, 173288, 257555, 214008, 730938, 868169, 731404, 667112, 135773, 34007, 1521706, 687679, 730534, 33621, 664233, 135125, 155345, 208991, 177827, 667353, 745003, 731685, 79729, 25664, 139242, 666140, 270549, 173200, 364777, 740381, 795309, 731423, 49888, 741891, 232714, 743290, 133531, 665379, 669319, 258127, 1238492, 682555, 726821, 1420676, 364510, 668186, 44193, 365045, 32917, 1174287, 725371, 119772, 730834, 252382, 179214, 1288183, 628418, 382791, 2384812, 364822, 726513, 282587, 731047, 25883, 134858, 207920, 668476, 251147, 667259, 38403, 365311, 178908, 1911930, 135379, 681875, 149809, 971276, 687667, 738558, 136952, 687287, 744606, 743118, 364352, 667375, 726647, 134978, 726675, 743394, 738424, 729929, 782688, 726782, 135303, 742695, 363955, 731348, 366889, 194350, 436554, 668536, 725340, 174861, 1859532, 261500, 714414, 667361, 742739, 364741, 366032, 729510, 745490, 731021, 342551, 740457, 726596, 2032639, 136121, 745072, 665086, 738966, 731073, 135407, 471664, 301122, 738506, 136747, 714437, 485104, 179733, 713913, 742542, 743760, 26651, 743519, 196992, 665392, 364717, 731277, 667067, 136868, 564878, 743245, 668089, 136399, 383718, 741954, 742590, 544683, 134976, 365589, 730300, 381854, 731305, 2348237, 669564, 714049, 366067, 487148, 668239, 1837488, 359202, 266361, 172785, 25895, 545475, 501527, 726981, 359051, 230126, 724642, 668815, 687852, 26455, 286138, 682507, 135539, 469549, 687953, 366085, 744395, 726353, 324666, 135352, 25865, 175772, 134671, 730699, 22908, 343987, 366842, 323438, 26519, 366042, 727067, 365121, 359461, 1104732, 135630, 135975, 238705, 472114, 502598, 645455, 669574, and 705147,
   (i-2) 43541, 687990, 365990, 562904, 725548, 365877, 742763, 180259, 2449149, 1089025, 713623, 382760, 258747, 743961, 174396, 724306, 1505308, 726454, 665452, 665833, 1352408, 731060, 73703, 207926, 489326, 1743279, 139073, 1626951, 1578721, 188393, 156720, 781738, 1645668, 714493, 1896337, 195340, 203166, 310519, 503874, 743722, 324066, 738944, 201467, 1870594, 122874, 140405, 204686, 2497617, 2306096, 1707527, 609631, 356960, 2345206, 740931, 342256, 725473, 682207, 2242054, 868577, 727154, 279470, 813174, 252489, 2341829, 729924, 263251, 250883, 738896, 1326169, 448163, 731648, 2113771, 365349, 731023, 744980, 668584, 727496, 132933, 592598, 46991, 128065, 730037, 356841, 2238108, 823680, 292042, 504539, 485803, 340657, 590264, 1084386, 742853, 210587, 884365, 134615, 740927, 1088781, 2091591, 667239, 727430, 486-401, 1286238, 742577, 1916307, 3054031, 366289, 366233, 80688, 665632, 379200, 162246, 743246, 486935, 287843, 365755, 811024, 489249, 840753, 731689, 50491, 1422194, 1251197, 1942634, 725268, 666410, 743415, 364448, 743367, 1287638, 668442, 28511, 1422791, 743224, 124701, 725612, 471568, 156183, 840687, 731751, 1705397, 26061, 666061, 725345, 740347, 346610, 131839, 123950, 2336358, 135801, 666425, 809959, 1550616, 2148946, 2728204, 366585, 667727, 116906, 288807, 345034, 768241, 667440, 1089513, 1131054, 1256485, 245426, 427786, 742696, 744911, 1013595, 1056782, 1057773, 1072914, 1086914, 110101, 1101773, 110938, 111460, 1116948, 1129745, 114008, 1146624, 117072, 1188588, 120649, 120916, 122063, 124554, 127360, 1285305, 129438, 1324060, 1337232, 134034, 137238, 137454, 1420842, 143957, 149910, 152398, 152802, 152844, 153411, 1541958, 1544175, 155345, 155717, 1578721, 1580147, 159381, 160163, 1632468, 1641901, 1654256, 1712870, 1735081, 1737165, 177967, 178984, 179857, 180950, 1844689, 187482, 1899942, 191881, 1929371, 1932202, 195712, 196115, 1982430, 200018, 202897, 204381, 209137, 2091389, 2103105, 2108870, 2137932, 21496, 2158322, 23121, 232002, 232507, 234357, 2349125, 235882, 235934, 236155, 2361911, 236386, 2364998, 2366175, 239086, 2398980, 239951, 243741, 248811, 251047, 255398, 259552, 271985, 276658, 276727, 285666, 285693, 289337, 292882, 295093, 3058557, 308564, 324140, 324225, 325008, 33285, 342416, 343867, 34783, 35356, 36371, 364204, 364271, 366359, 377560, 379540, 382612, 38775, 40633, 43629, 44064, 471101, 471210, 471947, 484504, 485085, 485725, 486201, 486204, 486779, 487103, 487130, 487436, 490710, 49725, 502658, 52740, 566585, 5725785, 665566, 665651, 665784, 665881, 665982, 666510, 666755, 667144, 667435, 667602, 668454, 668494, 669503, 713896, 714216, 723753, 723938, 724042, 724225, 724366, 724588, 725622, 725696, 725758, 725927, 726147, 726156, 726424, 726557, 726904, 727173, 727207, 727394, 727521, 727554, 729489, 730032, 730271, 730410, 730413, 730865, 730968, 730971, 731007, 731098, 731152, 731284, 731762, 738315, 739953, 740105, 740193, 740604, 740617, 740885, 740971, 741389, 741661, 741835, 741919, 741988, 742555, 742574, 742592, 742649, 742839, 742899, 742965, 742977, 743113, 743212, 743237, 743259, 743426, 744366, 744413, 744468, 744755, 744756, 744881, 744897, 744926, 745018, 745125, 745223, 745248, 745326, 773286, 782730, 78294, 80109, 812143, 824531, 825265, 838611, 851264, 924826, 950695, and 964736.
   (ii) marker genes specific for Grave's disease (GD that are contained as inserted DNA in the recombinant vectors selected from the group of vectors having the following IMAGE clone ID No:
   (ii-1) 25664, 868169, 731380, 1049033, 730942, 731616, 726578, 366388, 485872, 1662274, 2074228, 730926, 745106, 731021, 207920, 258865, 503189, 745490, 1103402, 1911930, 731305, 173296, 669181, 727491, 713879, 359461, 265558, 687667, 668239, 232366, 730814, 484535, 743774, 667587, 665086, 366889, 723950, 256177, 1859532, 665433, 985457, 564878, 376356, 383868, 471664, 724642, 23817, 724596, 487152, 669549, 155345, 347362, 666425, 24541, 290265, 667252, 725231, 343987, 705147, 1238492, 745072, 544683, 3054031, 744604, 731073, 2237263, 136919, 486-447, 740620, 1420676, 151247, 738558, 731645, 179288, 667683, 135125, 723914, 666110, 381854, 365056, 738970, 724554, 301122, 382521, 341763, 136747, 364717, 484577, 668510, 145696, 594279, 725340, 712118, 82687, 252291, 666551, 713859, 359051, 786662, 741497, 731298, 729957, 743903, 682555, 30981, 666778, 135407, 742919, 378458, 743579, 258127, 365613, 357807, 383528, 486189, 738938, 279172, 665649, 172828, 738332, 549054, 742695, 213529, 667048, 307094, 436554, 324079, 365526, 471574, 742580, 666277, 2348237, 485652, 744606, 52226, 236119, 730924, 173200, 342551, 744087, 345034, 731590, 743118, 261500, 136223, 668536, 666928, 730149, 180851, and 24623
   (ii-2) 687990, 743961, 486-493, 379279 and 667313, 726424, 122063, 729489, 724588, 343867, 1116948, 727154, 1632468, 1057773, 117072, 73703, 590264, 726147, 1089513, 724225, 80109, 744385, 1654256, 964736, 667435, 365349, 742696, 295093, 110938, 195712, 245426, 127360, 122874, 448163, 489326, 501476, 202897, 23121, 744911, 743212, 49725, 725548, 191881, 744926, 665452, 2158322, 727394, 811024, 259552, 741389, 232507, 342416, 2238108, 324225, 366359, 1188588, 742899, 823680, 782730, 204381, 727207, 243741, 120649, 366115, 740885, 726763, 258747, 727554, 153411, 665566, 1286238, 135773, 136686, 731711, 250771, 122063, 744385, 740140, 262894, 53081, 258698, 53119, 136366, 665674, 178161, and 729489,
   (iii) marker genes specific for macrofollicular adenoma (FTA-a) that are contained as inserted DNA in the recombinant vectors selected from the group of vectors having the following IMAGE clone ID No:
   (iii-1) 2348237, 135379, 743851, 1712992, 376699, 1251197, 1174287, 173841, 486221, 265345, 666564, 252382, 173371, 731073, 253386, 1285305, 342700, 179214, 3054031, 359797, 486189, 258865, 731742, 666061, 135705, 745072, 731044, 731616, 2148946, 258966, 504539, 725188, 213529, 742919, 682585, 666554, 825224, 1049033, 365045, 742837, 127507, 725198, 116906, 135875, 135773, 486282, 343987, 232366, 80162, 566862, 724544, 731115, 725454, 813174, 340657, 838611, 730534, 742763, 178255, 687336, 742542, 667683, 725473, 139073, 731202, 594279, 727289, 382423, 724556, 740457, 682276, 666361, 713879, 1016390, 667110, 359461, 25664, 743519, 486540, 1645668, 744899, 562904, 1174342, 258127, 1916307, 665693, 714109, 665649, 687270, 1942634, 1352408, 364686, 357442, 729957, 667598, 739247, 382738, 145696, 1578721, 544683, 745512, 249311, 705147, 727482, 365526, 724884, 1575056, 739432, 743224, 730779, 137238, 284828, 667048, 739237, 731047, 1326169, 364570, 174396, 665086, 235938, 666860, 743394, 758037, 740090, 594655, 760065, 731076, 1188588, 137478, 868169, 1012990, 382069, 666425, 135203, 341763, 669549, 726894, 731277, 485104, 668239, 682207, 741406, 201467, 1088781, 382002, 236155, 731758, 364934, 235882, 178792, 365056, 668152, 665452, 136646, 666110, 668146, 347503, 744616, 177857, 258747, 665156, 1308954, 471664, 485872, 587186, 136919, 504596, 265558, 135407, 156183, 484577, 725707, 173200, 137454, 743078, 155345, 666794, 667375, 135303, 134976, 301122, 741497, 725585, 743579, 263251, 744074, 42280, 742695, 731598, 666426, 743603, 665403, 188232, 1859532, 290265, 884365, 512910, 687953, 731357, 723950, 136747, 31924, 279470, 741790, 781738, 204686, 666334, 119939, 1085884, 489326, 356841, 743982, 1707527, 35628, 730814, 738358, 667252, 740927, 342581, 137131, 2150502, 2032639, 2306096, 767779, 743182, 309697, 744374, 2238108, 136223, 666279, 726821, 383945, 726618, 23817, 188393, 741474, 25895, 135980, 136014, 741066, 382521, 731275, 136598, 2345206, 382791, 724238, 485803, 725970, 682119, 276727, 668476, 713647, 742061, 1075949, 241481, 347362, 725548, 1492440, 1117183, 726600, 489249, 2345206, 327182, 133531, 2214396, 71428, 665342, 742743, 666235, 743259, 2030501, 666703, 179534, 744983, 356960, 1561035, 726647, 1741589, 137719, 2048801, 487148, 33664, 43541, 744895, 687667, 132752, 122874 and 2032639, 1116948, 129438, 195712, 1982430, 2103105, 21496, 2384812, 24065, 364271, 364717, 40633, 485085, 487379, 723755, 729875, and 753215,
   (iii-2) 745003, 1844968, 364324, 357201, 24392, 1420676, 687532, 724562, 729510, 725371, 24083, 738938, 730938, 148796, 137205, 174683, 21808, 3659591, 325155, 666792, 154172, 295412, 176543, 24532, 359202, 68950, 2046361, 725364, 741178, 668360, 300194, 23872, 364352, 364469, 726353, 742100, 23271, 667112, 25949, 22711, 744055, 430263, 738506, 738945, 743245, 812967, 744087, 250883, 741954, 667892, 364974, 744505, 187482, 52226, 23945, 179288, 738507, 1526058, 782688, 174861, 669295, 713859, 136868, 24300, 731726, 682251, 21716, 38789, 25883, 172693, 725340, 669141, 712295, 23641, 738944, 1716286, 723867, 726272, 23827, 357807, 136801, 135887, 2254555, 53333, 472111, 214008, 744106, 136462, 725533, 366042, 173820, 136114, 364575, 68972, 687679, 366436, 2237263, 32339, 26486, 1698236, 279150, 1101773, 666986, 669470, 257259, 667313, 128694, 178750, 2341829, 472160, 727123, 1087348, 135689, 135941, 151247, 172828, 172982, 208991, 33621, 376003, 502598, 545475, 549054, 666007, 667174, 667348, 724366, 724416, 729971, 731404, 739955, 740130, 740941, 742580, 743118, 744385, 744606, 744797, 745106, 745465, 78353, and 809828,
   (iv) marker genes specific for atypical follicular adenoma (FTA-aty) that are contained as inserted DNA in the recombinant vectors selected from the group of vectors having the following IMAGE clone ID No:
   (iv-1) 503874, 666128, 838611, 208991, 739097, 1089025, 725548, 739193, 155839, 28584, 202897, 743331, 743619, 725345, 345034, 235938, 729924, 681957, 30219, 773330, 665632, 2048801, 760065, 119939, 50491, 195340, 740941, 201467, 1581686, 743603, 207920, 731616, 71428, 30471, 504646, 487848, 342581, 1942634, 2238108, 740158, 42280, 196338, 742739, 743615, 1074708, 366484, 30981, 743246, 592598, 485104, 174396, 364777, 379540, 3054031, 123950, 1536968, 366100, 489326, 24587, 665086, 342256, 448163, 35628, 544818, 179534, 665156, 309645, 726454, 738896, 31924, 665452, 564878, 1286238, 379517, 177857, 744410, 730926, 809828, 288807, 740105, 366842, 146049, 713831, 140347, 666279, 341763, 665784, 24532, 33133, 365045, 1288183, 133531, 744926, 742763, 472111, 1012990, 743259, 1707527, 365121, 743270, 666315, 2484270, 471696, 364706, 364741, 80384, 590264, 285693, 173296, 1561035, 469549, 1422194, 731689, 667527, 1860115, 1087348, 204686, 1075949, 378813, 28511, 731422, 813174, 740931, 25883, 310519, 340657, 727289, 743722, 743367, 1662274, 364083, 136399, 731363, 743579, 1088781, 131839, 839101, 2449149, 1846326, 741406, 823590, 724014, 178750, 713623, 767779, 366067, 743078, 383966, 295412, 323438, 364729, 122874, 724642, 795309, 178255, 382760, 135941, 1102600, 69166, 231903, 682276, 1716286, 667533, 40304, 32917, 668452, 731060, 665820, 1624260, 1352408, 471664, 486401, 731722, 135689, 740604, 2139164, 739109, 135671, 666703, 433155, 687800, 502244, 73703, 484577, 1251197, 731202, 381032, 811024, 347503, 238840, 128065, 382791, 1870594, 258114, 731742, 727056, 781738, 487152, 731305, 731726, 342181, 745512, 356960, 365271, 78353, 730924, 742853, 2091591, 731023, 173200, 504596, 666792, 383718, 382002, 741474, 366032, 135766, 383945, 840687, 364436, 729957, 501527, 364686, 1089513, 111460, 127360, 136686, 140405, 1578721, 1735081, 1932202, 2091389, 23939, 251047, 377560, 380394, 384404, 471641, 487103, 487436, 609631, 714216, 723923, 724238, 726763, 729542, 731380, 731598, 738916, 741305, 742965, 742977, 743982, and 744074, 759865,
   (iv-2) 50202, 738970, 128694, 741891, 666671, 366388, 146976, 687667, 587186, 667727, 669136, 744001, 666361, 668300, 713647, 2074228, 282587, 179288, 687270, 726830, 666860, 687896, 364510, 324666, 300194, 268972, 135096, 565754, 136361, 300268, 713422, 758037, 713653, 366042, 723914, 172693, 208097, 549054, 730300, 175864, 178908, 687468, 134978, 729956, 503189, 681875, 1104732, 125552, 143759, 1877668, 2139315, 265558, 269873, 301122, 594279, 665376, 667174, 668239, 668536, 668815, 669188, 669564, 687782, 711918, 724276, 725454, 725791, 72744, 730938, 730971, 731751, 738332, 743774, and 745490,
   (v) marker genes specific for microfollicular adenoma (FTA-b) that are contained as inserted DNA in the recombinant vectors selected from the group of vectors having the following IMAGE clone ID No:
   (v-1) 740931; 731751; 840687; 666128; 379540; 667259; 725585; 503874; 23612; 3054031; 208097; 265558; 136976, 838611, 725707, 594279, 343987, 487474, 731128, 279172, 773330, 1089025, 725502, 2091591, 141216, 235882, 665452, 1251197, 236399, 50491, 376699, 727496, 725877, 682207, 687667, 729924, 179534, 724366, 30471, 743246, 1088781, 345034, 195340, 868577, 2238108, 2148946, 258114, 136919, 1870594, 1241974, 172828, 731348, 740158, 592598, 1326169, 809959, 741790, 725268, 119939, 135980, 136014, 740604, 730037, 448163, 668239, 512420, 258865, 365526, 667365, 742577, 116906, 258747, 738896, 342256, 486221, 288807, 743259, 136223, 725970, 342700, 327182, 264166, 1049033, 365801, 156720, 725473, 1074708, 290265, 687379, 71428, 1624260, 486401, 669564, 133531, 232388, 726454, 1705397, 487848, 809719, 251427, 287843, 1870594, 744926, 682119, 1641901, 742901, 1707527, 742853, 33133, 664233, 504596, 364204, 285693, 25664, 174396, 743722, 209137, 310519, 42280, 249311, 1505308, 727178, 667067, 731726, 46991, 137454, 740347, 666334, 666671, 359461, 487302, 1352408, 504646, 743118, 365919, 687800, 26061, 301122, 122874, 129438, 666235, 471664, 741497, 1117183, 364526, 175772, 179214, 471568, 744606, 1103402, 1942634, 727263, 484577, 745490, 31924, 682555, 665693, 823680, 1626951, 562904, 682311, 132752, 359797, 485803, 725548, 705147, 730012, 504539, 491004, 667440, 1917430, 743774, 666110, 309697, 724416, 135303, 731019, 1287638, 742743, 740718, 1645668, 378813, 1016390, 124701, 162246, 731738, 681890, 309645, 665376, 238840, 489326, 665632, 201467, 188232, 486189, 485085, 203166, 744374, 813174, 1743279, 742763, 156183, 669549, 376356, 665086, 2449149, 137719, 139073, 357442, 745083, 724544, 132933, 665433, 2497617, 22085, 364570, 713879, 731023, 713922, 252291, 1911930, 436554, 743415, 145696, 178348, 667239, 236155, 196992, 2484742, 781738, 210587, 78294, 110938, 114008, 117072, 131839, 153411, 155345, 160163, 178984, 191881, 200018, 234148, 234357, 236386, 239086, 289337, 486201, 486204, 487086, 591534, 627107, 665566, 667144, 713422, 713896, 724042, 725758, 730593, 738315, 741661, 741919, 742965, 743212, 743426, 745018, 812955, 884365, 1056782, 1072914, 1129745, 1131054, 1146624, 1188588, 1420842, 1544175, 1735081, and 2103105,
   (v-2) 724609, 365913, 666154, 267358, 665379, 667892, 251250, 713653, 365589, 1087348, 366042, 743146, 174654, 744657, 214008, 666007, 137005, 364436, 738558, 146976, 682175, 725533, 365121, 667112, 770858, 3659591, 469345, 154172, 731404, 730036, 135671, 739097, 739193, 743205, 811582, 364352, 725364, 668684, 725076, 666946, 136260, 971276, 2139164, 365271, 307025, 366254, 125552, 669263, 744439, 26519, 564994, 668815, 725335, 592276, 176543, 296702, 668152, 295412, 149809, 24176, 153667, 726675, 486510, 1526058, 366243, 148796, 194350, 143759, 33621, 668089, 669319, 1173570, 136399, 136686, 208991, 230126, 25883, 31120, 34007, 342551, 364324, 364469, 364569, 364741, 364844, 382612, 383718, 487475, 501476, 502598, 512910, 645455, 665649, 666315, 667514, 667533, 724276, 726353, 726763, 727056, 730938, 731227, 739576, 740130, 740620, 744385, 753215, 78353, 809828, and 813260,
   (vi) marker genes specific for follicular carcinoma (FTC) that are contained as inserted DNA in the recombinant vectors selected from the group of vectors having the following IMAGE clone ID No:
   (vi-1) 501476, 309009, 739193, 251452, 681957, 739097, 364448, 469549, 202897, 195340, 731380, 2345206, 23765, 471641, 502244, 713653, 486189, 740604, 682207, 740941, 469647, 743519, 743367, 966894, 727056, 2336358, 52897, 1326169, 773330, 139073, 725548, 137238, 742853, 838611, 22908, 207920, 375746, 738896, 152802, 343987, 743246, 1089025, 342256, 729924, 727496, 667514, 50491, 726454, 486401, 2238108, 730123, 342551, 1186334, 365589, 1837488, 1942634, 276727, 666986, 324066, 724014, 364083, 2449149, 744943, 258747, 276658, 265558, 731745, 726763, 28511, 235882, 1707527, 1896337, 448163, 301122, 724306, 687800, 669318, 2214396, 175268, 740347, 365271, 366074, 594279, 1942634, 366289, 665086, 1641901, 724366, 509606, 26061, 162246, 489249, 24541, 723923, 731751, 744410, 1870594, 743259, 731277, 381854, 25949, 489326, 491004, 2728204, 666703, 251147, 1870594, 484535, 258114, 26519, 487148, 726684, 149809, 731616, 252291, 71428, 364324, 156183, 25865, 1705397, 251250, 79729, 365562, 811024, 730926, 1085884, 366159, 682276, 364469, 668442, 741905, 356841, 730814, 177827, 42280, 724151, 743270, 136260, 485085, 742837, 666128, 666279, 727092, 364777, 179334, 757248, 2484270, 287843, 469345, 667061, 485803, 727491, 213113, 364846, 743619, 743987, 180640, 364706, 365177, 592598, 971276, 1581686, 713671, 129438, 1117183, 345034, 741066, 1286238, 178792, 740931, 744657, 740381, 2237263, 666154, 665538, 135671, 172996, 1698236, 768241, 188393, 1013595, 1057773, 1129745, 1131054, 1146624, 120649, 124554, 1420842, 153411, 155345, 1899942, 191881, 195712, 236055, 236386, 25077, 30219, 342416, 343867, 35356, 364436, 365121, 38775, 471574, 471834, 490710, 665327, 666928, 667250, 668815, 682451, 687468, 687551, 713896, 724588, 725345, 725696, 725758, 727178, 729489, 739953, 742935, 744897, 80109, and 964736,
   (vi-2) 782688, 136014, 135980, 725335, 724150, 307094, 723752, 510318, 726578, 324079, 2139164, 143759, 739237, 152655, 136646, 309697, 384087, 365408, 376699, 134671, 609631, 238840, 1074708, 366254, 742919, 257259, 840753, 668924, 136801, 743245, 731428, 731410, 738507, 430263, 744374, 1492440, 135887, 726782, 379517, 726849, 72744, 2467442, 743426, 135407, 135975, 137454, 238840, 486221, 669574, 266361, 134858, 731073, 359461, 744606, 731060, 666778, 726513, 236155, 744616, 744797, 23827, 743118, 609631, 665376, 742542, 725454, 135689, 713647, 743774, 724416, 687782, 723755, 726092, 731128, 782688, 80688, 53333, 22085, 46991, 486708, 730012, 236119, 549054, 2716748, 744797, 666784, 743043, 238840, 415554, and 729896,
   (vii) marker genes specific for multinodular goitres (MNG) that are contained as inserted DNA in the recombinant vectors selected from the group of vectors having the following IMAGE clone ID No:
   (vii-1) 284828, 173841, 667527, 202897, 469549, 207920, 469647, 363955, 323438, 276658, 263251, 731277, 35628, 741474, 743394, 212366, 258865, 724014, 742837, 1117183, 276727, 811582, 1743279, 666279, 666703, 725585, 714498, 525221, 739109, 731380, 382738, 1012990, 188232, 1012903, 726675, 356960, 235882, 208439, 178348, 727056, 124781, 300268, 868577, 327182, 249311, 266361, 241481, 376153, 745490, 31924, 235938, 687667, 44881, 725548, 383868, 342551, 382521, 667727, 365562, 359051, 136863, 773286, 236155, 744899, 366042, 383945, 723923, 340657, 668442, 382791, 666794, 121551, 346610, 232366, 110481, 128065, 379937, 731076, 682207, 50202, 725198, 208991, 173371, 724895, 742763, 666426, 743182, 381854, 347503, 743903, 838611, 155345, 2030501, 811024, 489326, 731645, 365521, 135671, 136747, 665433, 1705397, 731689, 724609, 740105, 731389, 1288183, 80162, 127507, 382002, 665086, 213113, 141216, 713913, 813174, 665495, 667110, 744395, 668300, 594279, 668510, 177857, 745512, 382069, 110101, 2242054, 310519, 38403, 3054031, 1188588, 665814, 667756, 173818, 731758, 2345206, 471568, 301122, 730814, 743270, 1859532, 382423, 725677, 744410, 723914, 1846326, 129438, 667514, 823680, 162246, 731115, 125552, 666367, 484535, 2113771, 667252, 744050, 562904, 666554, 667355, 137131, 760065, 727289, 731616, 230126, 725231, 135801, 512420, 594510, 40304, 1641901, 180259, 296702, 485872, 136919, 364741, 2148946, 364436, 136976, 1917430, 1049033, 264166, 741406, 196338, 1088980, 768241, 742061, 69166, 739237, 512910, 42280, 134416, 724238, 357442, 731598, 740347, 730834, 2384812, 724554, 201467, 1308954, 731305, 256177, 157847, 687336, 134615, 73778, 739851, 258966, 136801, 383718, 666334, 486221, 177775, 740457, 713608, 485085, 119772, 31120, 544683, 145696, 383528, 743619, 341763, 471664, 342581, 726647, 743982, 743290, 356841, 178255, 724812, 156183, 152847, 324066, 731742, 724884, 265558, 124701, 376983, 725188, 743016, 140405, 253386, 713879, 238840, 30471, 485803, 179266, 364885, 592276, 486189, 725473, 723950, 724537, 1645668, 137719, 1174287, 743517, 257555, 188393, 745123, 71428, 741497, 544818, 251427, 366085, 25865, 236119, 485104, 1570420, 174654, 745072, 1073288, 1116948, 204381, 255398, 259552, 501476, 645455, 731292, 744881, 745187, 745273, and 768246,
   (vii-2) 731202, 179534, 116906 and 1942634, 687625, 726782, 364324, 148796, 744505, 26164, 665668, 713837, 172828, 731726, 151247, 486356, 342181, 471641, 72744, 135689, 740941, 366032, 154172, 743987, 713422, 279172, 131839, 666551, 665392, 2055272, 729957, 258666, 743391, 53333, 109142, 743615, 133531, 309645, 68972, 682311, 666946, 194302, 26455, 744439, 739451, 137010, 669295, 325155, 731685, 172982, 738506, 51232, 78353, 742695, 1690788, 52226, 136114, 153667, 134858, 667587, 1420676, 1716286, 22711, 236399, 2467442, 250463, 250883, 257773, 25883, 295412, 30219, 345034, 486708, 668625, 724366, 725877, 726513, 726849, 730829, 739956, 740604, 741954, 743118, 743774, 744055, 744087, 744505, 744606, 745106, 782688, and 80688,
   (viii) marker genes specific for oncocytic adenoma (OTA) that are contained as inserted DNA in the recombinant vectors selected from the group of vectors having the following IMAGE clone ID No:
   (viii-1) 80688, 1870594, 731738, 1870594, 365348, 681875, 731751, 136114, 365161, 491004, 724366, 744983, 740718, 884365, 687381, 236399, 731726, 809719, 757248, 172982, 743774, 666410, 743961, 667239, 725502, 265345, 366436, 668584, 725076, 365349, 148796, 23612, 725726, 667361, 53333, 279470, 729510, 725877, 729956, 345034, 665784, 730300, 382612, 137454, 135689, 667310, 1860115, 731714, 365097, 729971, 731128, 682311, 549054, 726092, 609631, 366042, 714437, 68972, 741954, 725791, 730012, 669564, 666455, 238840, 173820, 743485, 726782, 235882, 743760, 364324, 823680, 179334, 729542, 135930, 738506, 155839, 416959, 682109, 175268, 26486, 725707, 725970, 667348, 669263, 28705, 725612, 162246, 666140, 738332, 365877, 740941, 727056, 838611, 731275, 310519, 384087, 174879, 744087, 727496, 379279, 25226, 731428, 365990, 364568, 2266583, 180082, 590264, 725836, 731648, 253386, 151247, 724562, 713837, 379517, 726454, 135887, 682088, 725340, 730606, 840753, 1422791, 723752, 669149, 741891, 669295, 382760, 208097, 665632, 731665, 486708, 285693, 1932168, 726578, 1896337, 250883, 713671, 730037, 740620, 682149, 116906, 669470, 1526058, 365090, 740245, 24541, 1326169, 486935, 365000, 727123, 1877668, 666066, 687306, 503874, 687541, 738944, 726849, 365609, 782688, 38789, 365271, 739901, 145112, 2381770, 742853, 484535, 356841, 743391, 365613, 738970, 364204, 740347, 742806, 152655, 2341829, 112819, 592598, 668089, 739956, 714049, 668684, 293916, 666128, 139242, 51232, 743118, 2728204, 565754, 727229, 2254555, 742543, 364448, 135096, 486401, 364575, 713831, 687990, 668924, 738896, 131839, 207926, 342551, 210587, 324079, 366388, 251216, 137005, 741894, 22085, 44193, 109142, 666361, 148212, 140405, 726272, 448163, 135875, 24392, 725401, 739247, 366289, 868577, 134858, 140347, 261656, 665376, 743246, 1420676, 726596, 357201, 668283, 172828, 487474, 665833, 309645, 376597, 731685, 2484742, 366089, 286138, 687912, 1536968, 666007, 667313, 1641901, 136361, 282587, 665156, 365311, 730123, 173119, 366842, 178750, 1707527, 364510, 364412, 366159, 668707, 279150, 739451, 365408, 745490, 667365, 510318, 2238108, 68950, 743043, 2306096, 740130, 669141, 743722, 22384, 682555, 682251, 669181, 743259, 208991, 342256, 744106, 666425, 738358, 726684, 365562, 745106, 665392, 731060, 687625, 23945, 1239535, 682528, 725268, 486493, 292042, 2467442, 134671, 486356, 665437, 713623, 666029, 379540, 1942634, 252489, 295412, 365121, 594279, 23827, 666517, 682479, 726779, 730829, 21808, 135450, 742100, 34007, 288807, 1016390, 114008, 366254, 713422, 730149, 469549, 135539, 665452, 725394, 665254, 759865, 667174, 744945, 726634, 172785, 2011082, 714013, 33621, 359461, 137131, 134978, 301122, 23271, 366115, 366074, 687270, 687667, 503189, 357807, 743415, 128694, 731398, 180259, 666235, 667353, 682555, 136868, 135991, 687287, 1741589, 52897, 687852, 781738, 26164, 152802, 1089025, 154172, 43541, 24300, 25949, 725677, 214611, 744439, 23872, 2032639, 730938, 743125, 2214396, 727251, 723923, 738938, 134785, 173288, 725364, 26455, 213113, 730036, 687896, 667587, 22908, 731404, 325155, 731423, 46991, 727457, 300194, 669136, 268972, 726513, 667598, 133531, 713653, 30219, 188232, 727263, 53081, 203166, 2237263, 740604, 743987, 738945, 669510, 123950, 26651, 665342, 724892, 687782, 194302, 307025, 727154, 1911930, 469647, 471641, 366032, 29541, 740224, 724150, 136598, 153667, 212366, 666778, 364526, 738966, 3659591, 22711, 1088781, 744505, 744606, 135203, 667061, 365177, 758037, 25883, 713647, 740580, 726353, 178908, 156720, 1844968, 137010, 136801, 724276, 24176, 740381, 137720, 725096, 179288, 665668, 1013595, 1057773, 1101773, 1129745, 123950, 125552, 1256485, 134034, 1352408, 146976, 149910, 1654256, 1737165, 187482, 196115, 214008, 234148, 2361911, 239951, 245426, 248811, 289337, 309009, 324225, 325008, 342416, 364959, 427786, 471101, 530069, 565317, 665881, 667602, 713896, 723753, 724042, 724588, 725511, 726106, 726424, 726904, 727173, 730037, 731152, 73703, 738315, 739953, 740885, 740971, 741835, 742696, 742977, 743237, 744055, 744366, 744413, 744468, 744755, 744756, 744911, and 745125,
   (viii-2) 1662274, 145696, 730926, 731115, 155345, 376356, 666794, 739109, 179534, 628418, 741406, 485872, 232388, 667110, 724895, 33133, 730834, 743270, 731380, 713913, 2048801, 485104, 731422, 124781, 731616, 724014, 668186, 731598, 1012990, 382738, 714498, 745072, 1942634, 342581, 347503, 731742, 724537, 365045, 738424, 724556, 730814, 666703, 341763, 739851, 744841, 744895, 726894, 512910, 384404, 382621, 744074, 258127, 364741, 136747, 1174342, 177967, 760065, 177857, 687468, 723950, 2384812, 504646, 740105, 365056, 137478, 378458, 753215, 724642, 731021, 3054031, 178255, 433155, 731076, 744410, 743603, 731722, 742046, 727289, 714493, 178792, 1581686, 1705397, 1308954, 366243, 667252, 731590, 724238, 730924, 504596, 544818, 35628, 486221, 381032, 501527, 258865, 741474, 724884, 276658, 1186334, 731227, 665649, 1859532, 564878, 71428, 666279, 668146, 731241, 257555, 381118, 665814, 726821, 364686, 208439, 82687, 1085884, 727296, 740457, 667527, 1671903, 201467, 135379, 795736, 1075949, 739432, 363955, 213529, 743331, 137238, 743619, 1241974, 632026, 730699, 743451, 665820, 725188, 731202, 379937, 487148, 1288183, 172996, 382069, 731277, 230126, 743982, 743579, 40304, 376983, 179266, 40134, 235938, 731689, 136863, 665433, 727067, 743078, 809959, 381854, 813174, 1846326, 382002, 2030501, 666334, 767779, 731305, 471829, 383868, 566862, 731758, 1624260, 1917430, 364777, 743519, 667355, 364729, 591907, 25865, 241481, 525221, 127507, 307094, 135352, 236119, 743811, 382791, 742061, 323438, 251147, 364706, 487152, 364717, 136686, 364885, 178348, 731363, 666085, 1174287, 667657, 347362, 743268, 196338, 173200, 486189, 665403, 487848, 745512, 731044, 472160, 713624, 177775, 173628, 594655, 744616, 364932, 157847, 744899, 681890, 745133, 713608, 740090, 727178, 743394, 471574, 471664, 486304, 731745, 366585, 1012903, 24587, 742837, 236155, 726600, 152847, 486540, 731019, 985457, 383718, 731645, 724554, 714414, 1578721, 786662, 383966, 261500, 119772, 743182, 366233, 364822, 132752, 364356, 31924, 366085, 38403, 668510, 1743279, 743290, 263251, 30471, 486447, 729929, 2242054, 382521, 296702, 173371, 383945, 121551, 179133, 136919, 666367, 740158, 682585, 1285305, 730942, 284828, 668329, 42280, 136121, 25664, 1561035, 379200, 669549, 668452, 726618, 256177, 724596, 743016, 135671, 177827, 364469, 124701, 485652, 667259, 712118, 346610, 743220, 1049033, 430471, 1086823, 1102600, 1103402, 134615, 135630, 156183, 2074228, 236331, 23876, 2484270, 266361, 276727, 340657, 366820, 377560, 384844, 40633, 484577, 485085, 485803, 487436, 502658, 587186, 665086, 666315, 666928, 667258, 669557, 725231, 729875, 731023, 731073, 738916, 739576, 740931, 742542, 742763, 742935, 743517, 744797, 773286, 80384, 811024, 813654, 823590, and 966894,
   (ix) marker genes specific for atypical oncocytic adenoma (OTA-aty) that are contained as inserted DNA in the recombinant vectors selected from the group of vectors having the following IMAGE clone ID No:
   (ix-1) 745003, 740130, 471641, 682088, 731404, 2254555, 2381770, 214008, 300268, 2467442, 125552, 666517, 366159, 740927, 665784, 24392, 727457, 148796, 590264, 665632, 345034, 840753, 309645, 743043, 731275, 51232, 667048, 838611, 773330, 884365, 667514, 364412, 366115, 757248, 146049, 140347, 342181, 727123, 30219, 155839, 382612, 723923, 682276, 682479, 743118, 740941, 356841, 53081, 745490, 687381, 324079, 173818, 731060, 743367, 365000, 669149, 285693, 731398, 110481, 726578, 687990, 23612, 723752, 713831, 1536968, 725076, 471702, 119939, 448163, 365271, 738506, 665674, 666128, 29541, 204686, 731665, 682251, 724126, 22908, 152802, 356960, 173119, 366289, 669141, 1420676, 682109, 301122, 357442, 174879, 868577, 25883, 288807, 366089, 471852, 162246, 742100, 743722, 666455, 265558, 744943, 379279, 668924, 203166, 194302, 729971, 724366, 682311, 667348, 365877, 731423, 713859, 730037, 23641, 713837, 376597, 135125, 669263, 123950, 172982, 1911930, 152655, 122874, 1084386, 839101, 251427, 666168, 112819, 565317, 1188588, 366889, 743485, 471568, 744505, 669510, 667361, 135689, 666564, 738332, 23765, 668584, 669319, 744087, 188393, 667587, 795309, 292042, 812967, 135450, 327182, 666946, 366032, 1707527, 667310, 52897, 137131, 366042, 738938, 725726, 667313, 665392, 1846326, 730149, 744606, 489249, 739237, 731047, 687541, 365161, 687800, 68950, 136260, 109142, 69166, 357807, 207926, 2091591, 1550616, 740381, 731714, 364575, 1286238, 364526, 151247, 136862, 1855110, 230882, 238840, 26519, 365121, 376462, 379517, 503189, 545475, 730300, 740620, 742935, 744439, 78353, 796513, and 813654,
   (ix-2) 724556, 668329, 173200, 174654, 731227, 1624260, 743078, 739576, 3054031, 119772, 487152, 740457, 667527, 382002, 382791, 2384812, 566862, 1075949, 730814, 235938, 745072, 341763, 383718, 486304, 379937, 666279, 241481, 731380, 743270, 1917430, 682585, 666703, 1049033, 1085884, 724642, 665086, 742061, 767779, 729875, 724014, 743603, 714498, 731277, 727289, 744895, 35628, 173200, 743619, 726821, 284828, 129438, 731616, 487103, 667355, 1012990, 487848, 471664, 744899, 42280, 713623, 667110, 502658, 730926, 484577, 1174287, 236155, 381854, 365056, 2030501, 731021, 177967, 667258, 525221, 743331, 135407, 738916, 669557, 382521, 731305, 730401, and 669098,
   (x) marker genes specific for oncocytic carcinoma (OTC) that are contained as inserted DNA in the recombinant vectors selected from the group of vectors having the following IMAGE clone ID No:
   (x-1) 727123, 724366, 366289, 471641, 501476, 744943, 2237263, 155839, 140347, 743774, 1716286, 365271, 236399, 731423, 252291, 812967, 739097, 471702, 1420676, 739193, 740347, 669564, 609631, 731751, 666671, 745490, 285693, 235882, 725791, 23612, 345034, 503874, 838611, 81911, 469647, 667514, 730012, 730300, 740941, 738358, 486493, 809719, 726684, 687667, 162246, 740620, 366436, 726454, 469549, 146976, 665784, 731738, 365349, 238840, 491004, 122874, 46991, 726763, 78353, 738896, 178908, 416959, 486401, 665632, 742590, 180259, 666455, 22711, 665674, 148796, 489326, 25883, 724306, 745003, 594279, 342256, 26455, 143759, 687852, 592598, 713422, 173820, 136560, 209137, 136361, 743205, 366042, 665538, 727056, 1837488, 782688, 744087, 725877, 2238108, 257259, 725836, 666154, 743246, 971276, 448163, 666410, 137720, 1844968, 73703, 770858, 2055272, 487474, 731023, 667348, 50491, 740130, 342551, 713623, 2032639, 723923, 2728204, 176543, 781738, 839101, 32339, 731357, 738970, 364204, 1089025, 364575, 34007, 135096, 364412, 743259, 1870594, 1101773, 366233, 342290, 366067, 565317, 742853, 29541, 682528, 80688, 731398, 713831, 309697, 744983, 187482, 665452, 665376, 359051, 139242, 1707527, 731726, 145112, 744055, 282587, 136114, 1526058, 366115, 123950, 743043, 416744, 1536968, 309645, 738332, 471696, 365090, 667310, 1088781, 149809, 173119, 724416, 743146, 109142, 1239535, 668815, 364510, 738938, 741954, 668089, 151247, 729542, 665833, 549054, 116906, 666946, 135941, 472111, 809828, 135450, 26651, 2115808, 713671, 743615, 731714, 666128, 366842, 724892, 730938, 22908, 324079, 365613, 231903, 743422, 687541, 668360, 287843, 489249, 342181, 207920, 364324, 682109, 743367, 2381770, 22085, 730123, 135875, 250883, 667061, 724126, 1521706, 687679, 208097, 745106, 38789, 682479, 744606, 668684, 726272, 743451, 25949, 744001, 1870594, 179334, 364568, 357807, 590264, 137353, 207926, 731298, 173288, 565754, 69166, 668584, 364926, 154172, 725726, 725340, 23765, 687625, 743391, 292042, 2032639, 128694, 258666, 382612, 725394, 687381, 469345, 730036, 180640, 188393, 52897, 295412, 51232, 884365, 744945, 174683, 195340, 279470, 731648, 665392, 730606, 379540, 43541, 725364, 731404, 729924, 137454, 665437, 253386, 359202, 731685, 364974, 135689, 727229, 2467442, 1417901, 725707, 204686, 741891, 53081, 134785, 682555, 22384, 134858, 379279, 135539, 300194, 2214396, 687270, 731060, 174861, 366388, 738944, 365288, 251216, 545475, 726779, 136868, 743118, 135352, 180082, 2113771, 178750, 731128, 179733, 137205, 740927, 376153, 365097, 30219, 1089025, 1129745, 131839, 160163, 175268, 1932168, 2349125, 239086, 252489, 267358, 359454, 365121, 427786, 471101, 484504, 487130, 502598, 665668, 665982, 667313, 724588, 725758, 726092, 726353, 739953, 739955, 739956, 740193, 743125, and 812143,
   (x-2) 731019, 366085, 258127, 382002, 714414, 486189, 725188, 1241974, 666703, 487152, 40304, 811024, 25865, 666279, 745133, 347362, 258966, 471664, 82687, 740931, 739109, 628418, 731742, 42280, 724014, 1012903, 1288183, 382423, 512910, 667527, 730942, 731021, 3054031, 724596, 179266, 742061, 667683, 743603, 71428, 713608, 173371, 1917430, 745072, 727289, 486221, 740457, 487848, 742542, 723755, 502658, 1012990, 729875, 25664, 731073, 666928, 668452, 486304, 1285305, 235938, 484577, 341763, 382791, 241481, 742837, 258865, 767779, 566862, 725454, 740105, 813654, 1174287, 1049033, 382738, 665433, 823590, 665086, 1645668, 645455, 668239, 741474, 364885, 236119, 35628, 682585, 1075949, 773286, 382069, 213529, 668536, 257555, 31924, 731380, 725231, 727251, 726821, 236155, 740158, 760065, 587186, 40633, 266361, 1085884, 152847, 669188, 363955, 813174, 284828, 134615, 255398, 343987, 731115, 730814, 667252, 174396, 364729, 1103402, 727178, 744899, 2048801, 382521, 39593, 813654, 666928, 175080, 667991, 136933, 723755, 173371, and 1917430,
   (xi) marker genes specific for papillary carcinoma (PTC) that are contained as inserted DNA in the recombinant vectors selected from the group of vectors having the following IMAGE clone ID No:
   (xi-1) 726763, 739193, 28584, 739097, 25883, 1716286, 365045, 666367, 379200, 365271, 731227, 726760, 384404, 208991, 744943, 1846326, 364729, 730926, 795736, 136686, 667117, 743517, 724151, 296702, 742837, 364777, 364706, 731616, 666986, 80384, 666007, 1837488, 383868, 40304, 366100, 744055, 136919, 230126, 1288183, 813174, 469647, 787938, 740158, 713608, 669318, 795309, 812967, 325155, 382791, 724596, 743703, 724238, 154172, 366484, 667657, 469549, 124781, 668452, 713831, 713913, 383718, 382621, 744616, 364686, 667533, 744074, 364822, 49888, 24587, 363955, 114008, 201467, 295412, 375746, 666315, 744385, 544818, 364846, 145696, 738424, 1624260, 485652, 1285305, 2484270, 194302, 770858, 366074, 823590, 724014, 379937, 194350, 727321, 366243, 68950, 364741, 731305, 1174342, 744895, 342181, 985457, 2384812, 727289, 731277, 1085884, 727296, 383966, 729924, 382738, 471574, 682585, 525221, 1102600, 665379, 753215, 359202, 1581686, 744797, 726513, 743982, 731722, 486304, 323438, 743422, 727491, 364436, 1844968, 666128, 30981, 382069, 3659591, 730829, 433155, 687953, 667176, 1671903, 364324, 726779, 668146, 743579, 26164, 740457, 307025, 667527, 135941, 744657, 665433, 731590, 378458, 723923, 136747, 136399, 744941, 365913, 730924, 376983, 669149, 682251, 2046361, 30471, 665403, 743043, 712118, 153667, 342290, 731422, 666703, 667067, 665814, 742743, 82687, 809828, 173628, 2345206, 471702, 179534, 730814, 172996, 668329, 471641, 665820, 731598, 136121, 148796, 743331, 1942634, 731758, 1352408, 309697, 731202, 472111, 501527, 78353, 741474, 738945, 178750, 743603, 724642, 33621, 364083, 381032, 745003, 366032, 971276, 177967, 609631, 725345, 745512, 71428, 485085, 743903, 744439, 743270, 738966, 196338, 731363, 743394, 366067, 727067, 745106, 31924, 724895, 34007, 744841, 52226, 731021, 682276, 687679, 135352, 1662274, 285693, 307094, 42280, 1308954, 668815, 665668, 232714, 1594414, 682119, 26455, 667110, 134785, 665674, 731380, 180640, 838611, 742046, 30219, 207920, 743016, 667259, 628418, 667252, 504646, 667361, 742061, 726675, 231903, 667892, 136863, 725533, 1917430, 137353, 744106, 714493, 32917, 714013, 342181, 501476, 730834, 79729, 666794, 2467442, 1698236, 665632, 743268, 741066, 726618, 743290, 665495, 73778, 744410, 669149, 381118, 809959, 276658, 632026, 122874, 744050, 731745, 1174287, 745123, 342581, 681890, 136462, 26651, 668186, 740090, 196992, 1578721, 745072, 669263, 729929, 726821, 740941, 1422194, 714414, 173820, 251250, 365056, 724609, 1075949, 512910, 132752, 740105, 2048801, 178255, 364717, 687379, 232388, 342181, 178908, 744604, 714498, 665342, 731241, 487152, 504158, 668476, 743619, 687800, 667355, 724812, 33133, 739432, 730938, 590264, 726634, 687532, 146049, 213529, 687896, 1626951, 284828, 1049033, 341763, 727056, 3054031, 726600, 156183, 731115, 471696, 143759, 366115, 383528, 731742, 31120, 174861, 668584, 743451, 112819, 2030501, 724556, 1101773, 137010, 2345206, 731076, 365000, 1707527, 366233, 366436, 1087348, 668360, 241481, 731404, 21716, 738938, 53333, 383945, 2055272, 682507, 1012990, 180259, 759865, 723950, 1086823, 2048801, 235938, 23876, 2467442, 35628, 366820, 382002, 384844, 430471, 485104, 502598, 667258, 669557, 724225, 738916, 742542, 742935, 767779, and 813260,
   (xi-2) 758037, 1932168, 489945, 738970, 667756, 265558, 2113771, 365562, 1712992, 739237, 135539, 159809, 268972, 135303, 1916307, 356841, 682088, 665437, 744945, 2728204, 324079, 727540, 470408, 740347, 364526, 343987, 730300, 594279, 162246, 549054, 743774, 510318, 136976, 725677, 667440, 757248, 140405, 2214396, 436554, 357807, 2306096, 668239, 365919, 665693, 125552, 725585, 823680, 359051, 1846326, 309009, 252291, 1877668, 1505308, 145105, 1741589, 327182, 188393, 743125, 132933, 52897, 731751, 359461, 173841, 212366, 768241, 324066, 730037, 235882, 668300, 2266583, 738358, 723914, 116906, 731738, 209137, 745490, 208097, 44193, 1743279, 110481, 24541, 727251, 745083, 1103402, 681957, 135689, 132935, 731128, 376153, 282587, 666361, 135875, 726636, 1641901, 1690788, 2011082, 366159, 32784, 22711, 713671, 44881, 1492440, 364448, 365526, 725707, 725454, 365348, 1896337, 251427, 725096, 137454, 727092, 357442, 725502, 152655, 668283, 136801, 43541, 667727, 725970, 266361, 135991, 365801, 1238492, 1417901, 731047, 665156, 175772, 141216, 365521, 742919, 743367, 731299, 730012, 489249, 744980, 301122, 713647, 416959, 23612, 128065, 236399, 726335, 366388, 731298, 384087, 300268, 134978, 279470, 740718, 665452, 208439, 738332, 726272, 136014, 135980, 731060, 742100, 364570, 487474, 868577, 1084386, 364412, 249311, 823680, 110101, 1104732, 119939, 1337232, 134858, 135975, 1860115, 188232, 234148, 236331, 251452, 253386, 33285, 33470, 345034, 364204, 364959, 366042, 378813, 379540, 38775, 472114, 486201, 486204, 665376, 669510, 669564, 687667, 705147, 711918, 724366, 725511, 725877, 726092, 726106, 726830, 741587, 741835, 744881, 809719, 812143, 812955, and 966894,
whereby an expression value is provided for each quantified marker gene expression level;
c) comparing (i) the expression value obtained at step b) for each disease-specific marker gene with (ii) a control expression value for the said marker gene, whereby a deregulated expression level of each disease-specific marker gene may be determined;
d) predicting or diagnosing the occurrence of a thyroid disease in the said patient if one or more disease-specific marker genes comprised in a group of marker genes, among the groups (i) to (xi) defined at step b), has a deregulated expression level; and
e) identifying, among the groups of disease-specific marker genes (i) to (xi) defined at step b), the group comprising the said one or more marker genes having a deregulated expression level, whereby the kind of specific thyroid disease that is predicted or diagnosed in the said patient is determined.

As used herein, the expression “disease-specific” means a relationship with a single specific thyroid disease. Thus, according to the invention, a “disease-specific” marker gene denotes a gene whose expression is deregulated only in patients that are affected by a specific thyroid disease. Preferably, thyroid diseases are selected from the group consisting of (i) autoimmune thyroiditis (AT), (ii) Grave's disease (GD), macrofollicular adenoma (FTA-a), (iv) atypical follicular adenoma (FTA-aty), (v) microfollicular adenoma (FTA-b), (vi) follicular carcinoma (FTC), (vii) multinodular goitres (MNG), (viii) oncocytic adenoma (OTA), (ix) atypical oncocytic adenoma (OTA-aty), (x) oncocytic carcinoma (OTC) and (xi) papillary carcinoma (PTC).

The disease-specific marker genes that are listed in groups (i-1), (ii-1), (iii-1), (iv-1), (v-1), (vi-1), (vii-1), (viii-1), (ix-1), (x-1) and (xi-1) are “under-expressed” in patients affected with the corresponding thyroid disease. Thus, in patients affected with the specified thyroid disease of a given group selected from (i-1) to (xi-1), the expression level of the said disease-specific marker genes is lower than the expression level of the same genes that is determined in patients which are not affected with the said given thyroid disease, including patients which are not affected with a thyroid disease.

The disease-specific marker genes that are listed in groups (i-2), (ii-2), (iii-2), (iv-2), (v-2), (vi-2), (vii-2), (viii-2), (ix-2), (x-2) and (xi-2) are “over-expressed” in patients affected with the corresponding thyroid disease. Thus, in patients affected with the specified thyroid disease of a given group selected from (i-2) to (xi-2), the expression level of the said disease-specific marker genes is higher than the expression level of the same genes that is determined in patients which are not affected with the said given thyroid disease, including patients which are not affected with a thyroid disease.

The in vitro prediction or diagnostic method above is termed an “integrated” method, since the said method allows the simultaneous testing of at least two disease-specific marker genes that are, when taken together, indicative of more than one thyroid disease. The integrated prediction or diagnosis method according to the invention thus allows to simultaneously test for the risk of occurrence of, or for the occurrence of, a thyroid disease, among all, or almost all, the known thyroid diseases. In some embodiments, the integrated method according to the invention allows the testing for the risk of occurrence of, or for the occurrence of, any one, or at least almost any one, thyroid disease among those that are already clinically classified.

Thus, according to the prediction or diagnosis method according to the invention, it is now possible to predict or diagnose the occurrence of any kind of thyroid disease, and especially any kind of follicular thyroid tumor, by performing a single test procedure using a unique biological sample from a patient.

The integrated in vitro method according to the invention consists of a prediction method, since the marker gene expression deregulation, that is determined after having performed the comparison step c), generally occurs very early during the course of the thyroid disease progression, and in most cases, if not in all cases, far before that any other detectable phenotypic change is available to the clinician's analysis who implements conventional clinical methods for diagnosing thyroid diseases, like those established by the World Health Organization (WHO), namely the WHO International Classification of diseases, including the WHO Classification of pituitary tumors.

The integrated in vitro method also consists of a diagnosis method, since it is shown herein a specific relationship between (i) a deregulation of the expression of each marker gene disclosed in the present specification and (ii) a particular kind of thyroid disease, as conventionally classified, e.g. according to the clinical analysis methods established by the WHO International Classification of diseases.

The “thyroid tissue sample” that is provided at step a) of the method consists of a sample from the thyroid tissue originating from the patient to be tested, which tissue sample comprises at least the minimum number of cells allowing the production of an amount of nucleic acid expression products (e.g. mRNA or cDNA) for performing step b) in optimal conditions. Generally, the thyroid tissue sample comprises at least 10³ cells, and preferably at least 10⁶ cells from the thyroid organ. Typically, the thyroid tissue sample that is provided at step a) of the method may be any of the specimens such as those taken by fine needle aspiration from a subject, or those prepared by excision and extirpation of a part of the thyroid.

At step b) of the method, quantifying the expression level of disease-specific marker genes encompasses determining an absolute or relative quantification value that illustrates the said marker genes expression activity. Quantification encompasses determining a quantification value for the mRNA synthesized by each of the disease-specific marker genes tested, or of the cDNA that may be obtained from the corresponding mRNA. As it will be specified further in the present specification, the quantification value that is determined at step b) of the method may consist of an absolute quantification value that reflects the amount of mRNA produced from each disease-specific marker gene tested that is present in the patient's thyroid tissue sample. In other embodiments of the method, the said quantification value may be expressed as a relative value, e.g. the ratio between (i) the amount of mRNA produced by the disease-specific marker gene tested and (ii) the amount of mRNA produced by a gene that is constitutively expressed, e.g. a house-keeping gene like actin.

As used herein, a “gene” encompasses, or alternatively consists of, a nucleic acid that is contained in the human genome and which is expressible, i.e. which is able to give rise to a corresponding mRNA. Thus, a “gene”, as used in the present specification, consists of a human genomic nucleic acid encoding a mRNA, whether or not the encoded mRNA codes for a polypeptide.

The identity of every disease-specific marker gene of interest that is described in the present specification is unequivocally defined as a gene, whose expression gives rise to a mRNA, and wherein the said mRNA (or its corresponding double-stranded cDNA) comprises the heterologous nucleic acid insert included in a recombinant vector that is unequivocally referenced by a “Clone ID No” in the I.M.A.G.E. collection of human cDNA clones (Integrated Molecular Analysis of Genomes and their Expression), which are mainly available under the form of arrayed cDNA libraries. Thus, a disease-specific marker gene is identified herein as a IMAGE clone ID number, which consists of the reference number of a specific clone that is publicly available upon request to the IMAGE Consortium or to agreed distributors, including (i) the American Type Culture Collection (ATCC, Manassas, Va., USA), (ii) Open Biosystems (Huntsville, Ala., USA) or (iii) Research Genetics/Invitrogen (Carlsbad, Calif., USA).

The one skilled in the art, on the sole basis of the IMAGE clone ID No reference for a disease-specific marker gene described herein, has thus a full access to the corresponding biological material, i.e. the recombinant vector that comprises, as an inserted cDNA, the nucleic acid sequence originating form the mRNA that is encoded by the corresponding disease-specific marker gene.

The disease-specific marker genes described herein are all available as IMAGE human cDNA clones, wherein the human cDNA is contained in the pT3T7 cloning vector, which consists of a well known pUC19-based phagemid vector comprising both the phage T3 and T7 promoters, and which is functional in E. coli.

The nucleic acid sequence of the human cDNA of interest that is inserted in each recombinant vector identified as an IMAGE clone ID No may be easily determined by the one skilled in the art, by using conventional techniques of DNA amplification and sequencing. Illustratively, for obtaining the nucleic acid sequence of the disease-specific marker genes described herein that are inserted in the pT3T7 cloning vector, the one skilled in the art may amplify the DNA insert using primers that specifically hybridize with the T3 promoter sequence (5′-AATTAATTAACCCTCACTAAAGGGT-3′—SEQ ID No 1) or with the T7 promoter sequence (5′-AGCTGTAATACGACTCACTATAGGG-3′—SEQ ID No 2), before sequencing.

Alternatively, the nucleic acid sequence of the human cDNA of interest that is inserted in each recombinant vector identified as an IMAGE clone ID No is directly available upon a query based on the IMAGE clone ID number disclosed herein at the IMAGE Consortium database, e.g. at the following Web address: http://image.llnl.gov/image/html/query_tools.shtml.

As a further alternative of access to the nucleic acid sequences of the disease-specific marker genes disclosed herein, the one skilled in the art may refer to the gene names corresponding to the IMAGE clone ID numbers that are listed in Tables 3-24 and 25 to 46, which gene names consist of the unequivocal name of each human gene that is attributed by the HUGO Gene Nomenclature Committee (HGNC). Nucleic acid sequences of the disease-specific marker genes disclosed herein are thus also available upon query at the HGCN database on the basis of the internationally recognized gene name, e.g. at the following Web address: http://www.gene.ucl.ac.uk/cgi-bin/nomenclature/searchgenes.pl.

Tables 3-24 and 25 to 46 list the disease-specific marker genes of interest whose expression level may be quantified, when performing the in vitro prediction or diagnosis method according to the invention.

Each of table 3 and 25 lists the disease-specific marker genes that are indicative of the occurrence of AT, if under-expressed.

Each of table 4 and 26 lists the disease-specific marker genes that are indicative of the occurrence of AT, if over-expressed.

Each of table 5 and 27 lists the disease-specific marker genes that are indicative of the occurrence of GD, if under-expressed.

Each of table 6 and 28 lists the disease-specific marker genes that are indicative of the occurrence of GD, if over-expressed.

Each of table 7 and 29 lists the disease-specific marker genes that are indicative of the occurrence of FTA-a, if under-expressed.

Each of table 8 and 30 lists the disease-specific marker genes that are indicative of the occurrence of FTA-a, if over-expressed.

Each of table 9 and 31 lists the disease-specific marker genes that are indicative of the occurrence of FTA-atypical, if under-expressed.

Each of table 10 and 32 lists the disease-specific marker genes that are indicative of the occurrence of FTA-atypical, if over-expressed.

Each of table 11 and 33 lists the disease-specific marker genes that are indicative of the occurrence of FTA-b, if under-expressed.

Each of table 12 and 34 lists the disease-specific marker genes that are indicative of the occurrence of FTA-b, if over-expressed.

Each of table 13 and 35 lists the disease-specific marker genes that are indicative of the occurrence of FTC, if under-expressed.

Each of table 14 and 36 lists the disease-specific marker genes that are indicative of the occurrence of FTC, if over-expressed.

Each of table 15 and 37 lists the disease-specific marker genes that are indicative of the occurrence of MNG, if under-expressed.

Each of table 16 and 38 lists the disease-specific marker genes that are indicative of the occurrence of MNG, if over-expressed.

Each of table 17 and 39 lists the disease-specific marker genes that are indicative of the occurrence of OTA, if under-expressed.

Each of table 18 and 40 lists the disease-specific marker genes that are indicative of the occurrence of OTA, if over-expressed.

Each of table 19 and 41 lists the disease-specific marker genes that are indicative of the occurrence of OTA atypical, if under-expressed.

Each of table 20 and 42 lists the disease-specific marker genes that are indicative of the occurrence of AT, if over-expressed.

Each of table 21 and 43 lists the disease-specific marker genes that are indicative of the occurrence of OTC, if under-expressed.

Each of table 22 and 44 list the disease-specific marker genes that are indicative of the occurrence of OTC, if over-expressed.

Each of table 23 and 45 lists the disease-specific marker genes that are indicative of the occurrence of PTC, if under-expressed.

Each of table 24 and 46 lists the disease-specific marker genes that are indicative of the occurrence of PTC, if over-expressed.

Tables 3-24 and 25-46 show the results obtained from two distinct sets of determination of the pertinent disease-specific markers.

In each of Tables 3-24 and 25-46, the left column (“name”) indicates, when available, the disease-specific marker gene name, according to the HUGO international nomenclature. When the international nomenclature gene name was not available, the IMAGE clone ID no is indicated. The second column (“cloid”) indicates the IMAGE clone ID no of the recombinant vector comprising, inserted therein, the cDNA encoding the disease-specific marker gene that is publicly available upon request near the IMAGE Consortium or near an agreed distributor Company. The third column (“p value”) indicates the statistical relevance of the relationship between (i) a deregulated expression of the disease-specific marker gene and (ii) the occurrence of the thyroid disease. The right column (“fold”) indicates the mean fold over-expression or under-expression of the disease-specific marker gene that has been experimentally determined, as compared to the “control” expression level value that has been determined from thyroid tissue samples originating from patients which are not affected with the specific thyroid disease, which include patients which are affected with a distinct thyroid disease, as well as patients which are not affected with any thyroid disease.

In all cases, the one skilled in the art may design detection and/or quantification means, including nucleic acid primers or probes, specific for every one of the marker genes of interest described herein, on the basis of the IMAGE clone cDNA material and also on the basis of their sequences that are available from the various sequence databases that are referred to above.

Illustratively pair of primers that specifically hybridise with the target nucleic acid gene marker of interest may be designed by any one of the numerous methods known in the art, based on the known partial or complete sequence of the said marker gene.

In certain embodiments, for each of the marker genes of the invention, at least one pair of specific primers, as well as the corresponding detection nucleic acid probe, is already referenced and entirely described in the public “Quantitative PCR primer database”, notably at the following Internet address: “http://lpgws.nci.nih.gov/cgi-bin/PrimerViewer”.

In all cases, the one skilled in the art may design nucleic acid primers or probes that specifically hybridise with each of the marker genes described herein, starting from their known 3′-end and/or 5′-end nucleic acid sequences.

In other embodiments, a specific pair of primers may be designed using the method disclosed in the U.S. Pat. No. 6,892,141 to Nakae et al., the entire disclosure of which is herein incorporated by reference.

Examples of polynucleotides that are directly usable as primers or probes, or alternatively are usable for designing primers or probes, for the purpose of the invention consist of the polynucleotides having the nucleic acid sequences SEQ ID No 3 to 486, each polynucleotide being specific for a given disease-specific marker gene described herein, as it will be detailed elsewhere in the present specification.

As disclosed in the examples herein, the inventors have performed a wide range differential expression analysis of more than 8000 candidate genes on thyroid tissue samples previously collected from patients affected with a thyroid disease. The collection of thyroid tissue samples that has been used for performing the differential expression analysis comprised various sets of thyroid tissue samples, each set of thyroid tissue samples comprising three or more tissue samples originating from the same number of patients for which a specific thyroid disease had been clinically diagnosed, and the whole sets of thyroid tissue samples covering all, or at least almost all, thyroid diseases that are presently known in the art.

The wide range differential expression analysis of more than 8000 candidate genes was performed by:
(i) manufacturing DNA microarrays by immobilization on a solid support of labeled candidate gene-specific probes, each labeled probe being previously prepared by PCR amplification of each of the more than 8000 IMAGE clone vectors, each IMAGE clone vector containing a candidate gene nucleic acid. Each labeled probe has the ability to specifically hybridize with an expression product of the corresponding candidate gene;
(ii) (ii-a) extracting the total RNA material from each thyroid tissue sample previously collected from patients affected with a thyroid disease, and (ii-b) obtaining labeled cDNAs from the total population of mRNAs extracted at step (ii-a). Total RNA extracted from individuals that are not affected with a thyroid disease is also used, as further controls.
(iii) hybridizing the labeled cDNAs obtained at step (ii) with the DNA microarrays manufactured at step (i);
(iv) measuring the signal intensities that are generated after the formation of hybrid complexes between (i) a plurality of probes immobilized on the DNA microarrays and (ii) a plurality of labeled cDNAs, whereby an expression value for each candidate gene is obtained. and
(v) determining:
   (v-1) for each of the candidate gene tested, if there exists a relevant relationship between the expression level value of the said candidate gene, as measured at step (iv), and the occurrence of the thyroid disease, or
   (v-2) for sets of two or more of the candidate genes tested, if there exists a relevant relationship between the expression level value of the said candidate gene, as measured at step (iv), and the occurrence of the thyroid disease

The determination of a relevant relationship between the expression level value of the said candidate gene, at step (v-2) above, and the occurrence of the thyroid disease, may be performed by any one of the methods of the suitable statistical analysis that are well known from the one skilled in the art.

The determination of a relevant relationship between the expression level values of a set of two or more candidate genes, as measured at step (iv) above, and the occurrence of the thyroid disease was performed as it follows:
(1) (1-a) performing a one-way analysis of variance (ANOVA) for detecting differences between the 12 class means (i.e. individuals affected with a thyroid disease among the 11 thyroid disease classes (i) to (xi) previously described, and individuals not affected with a thyroid disease). (1-b) lowering the incidence of multiple testing effects, using the false discovery rate (FDR) controlling procedure. (1-c) determining gene expression specificities with t-tests, by comparing mean class expression with gene profiles.
(2) scoring each thyroid tissue sample by its similarity to the corresponding class centroids.
Steps (1) and (2) allow correcting the raw expression level measurements that were performed at step (iv) of the method above.
(3) evaluating the thyroid disease prediction power of the candidate genes tested, by calculating, for sets of two or more candidate genes, a predictive score which is assigned after comparing the statistical relevance values of the said sets of candidate genes obtained by one or more statistical method, such as one or more statistical method selected from the group consisting of (i) the Linear Diagonal Discriminant Analysis (LDDA), (ii) the Support Vector Machine (SVM), and (iii) the Nearest Centroid (NC). The predictive score that is finally calculated for each set of candidate genes may consist of a p value, in which case the statistical relevancy of a given candidate gene for predicting a given thyroid disease increases with decreasing values of the p value predictive score.
(4) selecting a candidate gene as a disease-specific marker gene if the assigned score value indicates a statistical relevancy which is higher than a threshold statistical relevancy (predictive score threshold value).

According to the invention a candidate gene consists of a disease-specific marker gene if its predictive score value, calculated as a p value, is lower than 0.05, when using the method generally disclosed above and detailed in the examples herein.

According to the invention, a candidate gene can also be defined as a gene for which the expression profiles, in association to another candidate gene, show high predictive potential in selection method (3) above.

According to another embodiment, a set of candidate genes consists of a set of disease-specific marker gene if its predictive score value, calculated as a p value, is lower than 0.05, when using the method generally disclosed above and detailed in the examples herein.

A complete list of the candidate genes that have been finally selected as disease-specific genes according to the invention is disclosed in Tables 3-24. The most disease-specific marker genes that are listed in Tables 3-24 have a p value that is lower than 0.05, when the p value is assessed by the method described in the examples herein.

Thus, in all embodiments of the in vitro prediction or diagnosis method according to the invention, the two or more disease-specific marker genes that are quantified at step b) mainly consist of marker genes having a p value predictive score lower than 0.05, as shown in Tables 3-24.

The accuracy of the prediction or the diagnosis results that are obtained at steps d) and e) of the in vitro method may increase with an increasing number of disease-specific marker genes belonging to a single thyroid disease class (i) to (xi) that are quantified at step b).

Depending on the accuracy of the prediction or of the diagnosis results which is sought, when performing the in vitro prediction or diagnosis method according to the invention, the one skilled in the art will adapt step b), so as to use a suitable combination of (1) the number of disease-specific marker genes to be quantified for a specific thyroid disease class among classes (i) to (xi) defined previously and (2) the predictive score (e.g. the p value) of the disease-specific marker genes to be quantified.

As it is shown in Tables 3-24 and 25-46, for a given class of thyroid disease among the classes (i) to (xi) previously defined herein, (a) a part of the disease-specific marker genes may be over-expressed, and (b) the remaining part of the disease-specific marker genes may be under-expressed, as compared to its expression level value found for all other thyroid disease classes as well as for healthy individuals.

At step b) of the in vitro prediction or diagnosis method according to the invention, the number of disease-specific marker genes that are quantified in each group (i) to (xi) is of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199; 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746 or 747, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, or 900 it being understood that the maximum number of disease-specific marker genes is limited by the number of disease-specific market genes that are described herein in each of the groups (i) to (xi).

As it will be detailed further in the specification, the comparison step c) is performed using a “control” expression value for each disease-specific marker gene that is tested. The said control expression value consists of the mean expression value for the said disease-specific marker gene that is found in individuals that are not affected with the thyroid disease to which a deregulation of the said marker gene is associated.

In some preferred embodiments, the comparison step c) consists of comparing the expression value of a plurality of marker genes that are known to be deregulated in patients affected with one of the thyroid diseases, selected from the thyroid diseases (i) to (xi), with the reference expression values for each of the said marker genes that are previously determined in patients that are not affected with the same thyroid disease, including healthy individuals that are not affected with any thyroid disease. The collection of expression values for the tested marker genes may also be termed an “expression profile” of these genes. In these preferred embodiments step c) encompasses comparing (i) the expression profile of the gene markers of interest that has been determined from the biological sample of the patient under testing with (ii) at least one reference expression profile of the same marker genes that has been previously determined from patients that are not affected with the said thyroid disease selected from the thyroid disease (i) to (xi). In certain of these preferred embodiments step c) consists of comparing (i) the expression profile of the gene markers of interest that has been determined from the biological sample of the patient under testing with (ii) at every reference expression profile of the same marker genes that has been previously determined from patients that are not affected with the said thyroid disease selected from the thyroid disease (i) to (xi), which may also include patients that are not effected with any thyroid disease. Then, at step d), a thyroid disease is predicted if the “experimental” expression profile that is determined for the patient tested is similar with one reference expression profile corresponding to patients affected with a specific thyroid disease selected from the thyroid diseases (i) to (xi). Then, at step e), the kind of specific thyroid disease is predicted, which consists of the thyroid disease for which the corresponding reference expression profile is the most close to the experimental expression profile that has been determined for the patient tested.

In certain embodiments of the method, the expression level value of a disease-specific marker gene can be provided as a relative expression level value. To determine a relative expression level value of a disease-specific marker gene, illustratively a disease-specific marker gene belonging to group (i) described herein, the level of expression of the said marker gene is previously determined for 10 or more samples of thyroid tissue originating from patients who are not affected with autoimmune thyroiditis (AT), prior to the determination of the expression level for the sample in question. The median expression level of the said disease-specific marker gene belonging to group (i), which has been determined in the larger number of samples, is determined and this median expression value is used as a baseline expression level, that may also be termed “control” value for the said disease-specific marker gene. The expression level of the said disease-specific marker gene determined for the test sample (absolute level of expression) is then divided by the median expression value obtained for that marker. This provides a relative expression level.

For a given disease-specific marker gene whose expression level is quantified at step b), a deregulated expression level is determined at step c) if there exists a relevant difference between (i) the expression value obtained at step b) and (ii) the reference control value. The said relevant difference may be any expression level difference that is found statistically significant, irrespective of the statistical method that is used. Generally, a relevant difference is found if, either:
(i) the deregulated expression of the disease-specific marker gene consists of an under-expression, as compared to the control expression value, the deregulated expression value being 0.98 fold or less the control expression value; or
(ii) the deregulated expression of the disease-specific marker gene consists of an over-expression, as compared to the control expression value with a p value <0.05.

At step d) of the in vitro prediction or diagnosis method according to the invention, the occurrence of a thyroid disease is predicted or diagnosed if a deregulated expression level of one or more disease-specific marker genes belonging to one group of marker genes selected from groups (i) to (xi) has been determined at step c).

Generally, if a deregulated expression level for one or more disease-specific marker genes is determined at step c), the said one or more disease-specific marker genes whose expression is deregulated all belong to the same group of marker genes, among groups (i) to (xi) described herein. Incidentally, errors may be introduced when performing steps b) or c) of the method, leading to the determination of a deregulated expression level of marker genes belonging to more than one group of marker genes, among groups (i) to (xi) described herein. The incidence of such artifacts may be easily avoided if, for each group (i) to (xi) for which marker genes expression level is quantified at step b), more than one disease-specific marker gene is included.

Thus, at step b), for each of the groups (i) to (xi) that is tested, preferably the expression level of 2 or more disease-specific marker genes, more preferably the expression level of 5 or more disease-specific marker genes, and most preferably the expression level of 12 or more disease-specific marker genes, is quantified, so as to lower the incidence of eventual technical artifacts which may occur when performing the in vitro prediction or diagnosis method according to the invention.

As already mentioned above, the in vitro prediction or diagnosis method according to the invention allows the prediction or the diagnosis of a thyroid disease selected from the groups (i) to (xi) of thyroid diseases described herein. In some embodiments, the said in vitro method allows the prediction or the diagnosis of all, or almost all, known thyroid diseases, if a simultaneous testing of marker genes included in a plurality of groups of disease-specific marker genes, among groups (i) to (xi), is performed, at step b) of the method.

Thus, in certain embodiments of the in vitro prediction or diagnosis method according to the invention, step b) consists of quantifying the expression level of marker genes included in more than two groups of disease-specific marker genes, selected from groups (i) to (xi) described herein. In certain embodiments of the method, step b) comprises quantifying the expression level of marker genes included in 3, 4, 5, 6, 7, 8, 9, 10 or 11 groups of disease-specific marker genes, selected from groups (i) to (xi) described herein.

The occurrence of every kind of thyroid disease, or at least almost every kind of thyroid disease, may be predicted or diagnosed when using the in vitro prediction or diagnosis method according to the invention, if, at step b), the expression level of one or more disease-specific marker genes in each of the groups (i) to (xi) of marker genes is quantified.

Thus, in certain preferred embodiments of the in vitro prediction or diagnosis method according to the invention, step b) comprises quantifying the expression level of disease-specific marker genes selected from each of groups (i) to (xi) described herein.

At step e) of the in vitro prediction or diagnosis method according to the invention, the kind of thyroid disease that is affecting the tested patient is determined by simply assessing to which group of disease-specific marker genes, among the groups (i) to (xi) described herein, belong the marker gene(s) for which a deregulated expression level has been determined at step c). Illustratively, if a deregulated expression level has been determined at step c) for one more disease-specific marker genes belonging to group (vi), then the occurrence of a follicular carcinoma (FTC) is predicted or diagnosed for the patient tested.

In the embodiments wherein, at step b) of the method, it is quantified the expression level of less than every one of the disease-specific marker genes belonging to a specific group, among groups (i) to (xi) described herein, then it is quantified preferably highly significant marker genes of the said specific group, so as to counterbalance the use of an eventual low number of disease-specific marker genes, for the said specific group.

In certain preferred embodiments of the prediction or diagnosis method according to the invention, the two or more disease-specific marker genes are selected from two or more of the groups of marker genes consisting of:
(i) marker genes specific for autoimmune thyroiditis (AT) that are contained as inserted DNA in the recombinant vectors selected from the group of vectors having the following IMAGE clone ID No:
(i-1) 154172, 78353, 135941, 545475, 1087348, 744055, 667174, 592276, 135975, 214008, 295412, 549054, 813260, 565317, 787938, 739956, 1104732, 135630, 667892, 725096, 72744, 645455, 744439, 143759, 669574, 136686, 2266583, 2074228, 364412, 724276, 364959, 669510, 742580, 727251, 300268, 713422, 724416, 31120, 811582, 564994, 1877668, 28584, 2055272, 137720, 687782, 753215, 173200, 486708, 1716286, 503189, 839101, 471641, 472114, 726830, 730938, 744797, 472160, 809828, 2467442, 668186, 669318, 738970, 324079, 33621, 364706, 682088, 591907, 744606, 738332, 51232, 502598, 739193, 741954, 744616, 724596, 364469, 2384812, 34007, 739097, 666778, 667355, 713647, 26519, 731404, 665376, 745106, 744385, 713653, 666007, 725364, 136399, 364729, 23827, 705147, 668452, 669263, 208991, 135407, 742542, 669188, 135689, 738938, 512910, 471574, 22711, 782688, 743118, 726821, 364777, 365045, 740941, 744087, 364324, 309697, 730829, 665649, 136014, 668815, 359461, 366032, 726782, 731073, 1837488, and 238705,
(i-2) 665833, 1645668, 731060, 1705397, 741835, 609631, 2113771, 1337232, 713623, 35356, 122063, 40633, 812143, 811024, 137238, 665881, 140405, 377560, 740193, 727521, 731007, 740604, 665566, 731098, 285666, 255398, 731023, 1286238, 1084386, 730037, 742763, 202897, 726424, 740971, 73703, 120916, 487436, 730968, 471947, 324140, 730032, 666510, 356960, 123950, 485803, 308564, 730971, 1089513, 110938, 201467, 727554, 669503, 730413, 179857, 1899942, 276727, 188393, 234357, 566585, 239951, 36371, 156183, 23121, 471101, 1086914, 21496, 180259, 727173, 342416, 1088781, 486201, 325008, 813174, 2349125, 484504, 2366175, 340657, 366289, 127360, 742574, 744926, 562904, 487103, 743722, 781738, 1632468, 727394, 143957, 251047, 1116948, 2398980, 34783, 1982430, 195712, 1932202, 3058557, 2091389, 5725785, 50491, 366359, 743212, 111460, 741988, 1735081, 825265, 682207, 46991, 1844689, 78294, 258747, 744468, 448163, 724225, 1544175, 742965, 964736, 742839, 851264, 730410, 740931, 152398, 489326, 276658, 1013595, 1737165, 2158322, 162246, 738315, 744413, 727496, 742555, 324225, 134034, 731152, 33285, 486779, 295093, 178984, 725548, 485725, 744366, 159381, 782730, 259552, 503874, 1146624, 174396, 120649, 743113, 744911, 114008, 743237, 1072914, 742899, 153411, 725696, 487130, 155345, 160163, 2137932, 743246, 592598, 490710, 745018, 245426, 1324060, 726454, 740885, 1131054, 191881, 924826, 2238108, 950695, 731762, 714216, 486401, 1654256, 235934, 1089025, 730271, 243741, 742592, 1580147, 668494, 745248, 2361911, 427786, 744755, 726904, 745223, 738896, 743259, 80109, 1712870, 232002, 742696, 342256, 744897, 726557, 741389, 1420842, 725758, 1707527, 289337, 739953, 239086, 713896, 236386, 1129745, 2364998, 504539, 110101, 1057773, 128065, 725345, 1896337, 726156, 731284, 292882, 288807, 152844, 149910, 726147, 152802, 725927, 196115, 1578721, 210587, 742977, 200018, 744756, 741661, 667239, 725473, 2103105, 343867, 364271, 122874, 180950, 232507, 285693, 723938, 665651, 665632, 1326169, 204381, 727207, 665452, 117072, 271985, 1188588, 1929371, 665982, 471210, 824531, 1352408, 667435, 248811, 742649, 741919, 742853, 1541958, 1056782, 668454, 1256485, 49725, 43629, 730865, 667144, 724042, 667602, 745326, 725622, 723753, 724588, 744881, 485085, 745125, 823680, 666755, 155717, 727154, 1870594, 740347, 729489, 345034, 252489, 725268, 124554, 44064, 134615, 379540, 590264, 2108870, 2306096, 80688, 884365, 502658, 1285305, 1641901, 356841, 773286, 868577, 177967, 364204, 204686, 250883, 310519, 743367, 740105, 665784, 724366, 235882, 129438, 38775, 382612, 666410, 486204, 187482, 236155, 137454, 1101773, 743426, 838611, 209137, 666061, 52740, 740617, 2108870, 665651, 1844689, 1352408, 1056782, 668454, 43629, and 155717,
(ii) marker genes specific for Grave's disease (GD that are contained as inserted DNA in the recombinant vectors selected from the group of vectors having the following IMAGE clone ID No:
(ii-1) 724596, 503189, 135407, 731021, 364717, 23817, 24623, 724596, and 180851,
(ii-2) 726424, 122063, 729489, 724588, 343867, 1116948, 727154, 1632468, 1057773, 117072, 73703, 486493, 590264, 726147, 1089513, 724225, 80109, 744385, 1654256, 964736, 667435, 365349, 742696, 295093, 110938, 195712, 245426, 127360, 122874, 448163, 489326, 501476, 202897, 23121, 744911, 743212, 49725, 725548, 191881, 744926, 665452, 2158322, 727394, 811024, 259552, 741389, 232507, 342416, 2238108, 667313, 324225, 366359, 1188588, 742899, 823680, 782730, 204381, 727207, 243741, 120649, 366115, 740885, 726763, 258747, 727554, 153411, 665566, 1286238, 135773, 136686, 731711, 250771, 122063, 744385, 740140, 262894, 53081, 258698, 53119, 136366, 665674, 178161, and 729489,
(iii) marker genes specific for macrofollicular adenoma (FTA-a) that are contained as inserted DNA in the recombinant vectors selected from the group of vectors having the following IMAGE clone ID No:
(iii-1) 486221, 1174287, 666061, 504539, 742837, 173371, 486189, 682585, 258966, 253386, 213529, 566862, 485085, 723755, 382423, 731742, 1285305, 235882, 137478, 731115, 727289, 731047, 236155, 705147, 1049033, 188232, 666794, 382069, 119939, 742763, 731073, 284828, 743603, 665649, 745072, 562904, 40633, 725473, 744616, 356841, 340657, 129438, 485872, 365045, 767779, 587186, 135407, 1645668, 744899, 382521, 382738, 364271, 667110, 682276, 726821, 25664, 21496, 729875, 665086, 178255, 725454, 71428, 382002, 365056, 1016390, 731616, 1578721, 724238, 1012990, 2103105, 2384812, 258865, 341763, 364717, 1116948, 1326169, 741474, 42280, 753215, 1982430, 731277, 31924, 195712, 742542, 665452, 1174287, 487379, and 24065,
(iii-2) 739955, 1420676, 745003, 738938, 300194, 25883, 295412, 364324, 730938, 744055, 725371, 1844968, 22711, 250883, 741954, 744087, 187482, 744505, 1101773, 725364, 742100, 726353, 173820, 782688, 724366, 731726, 154172, 53333, 1716286, 745106, 667313, 208991, 744385, 809828, 135689, 744797, 135941, 366042, 172982, 23827, 549054, 214008, 666007, 136801, 724416, 545475, 667892, 729971, 742580, 740941, 743118, 33621, 364469, 731404, 667348, 78353, 151247, 472160, 1087348, 502598, 744606, 172828, 740130, 667174, 745465, 173820, 24392, 300194, 208991, 24532, 725371, 22711, 376003, 1844968, 359202, 295412, and 25883,
(iv) marker genes specific for atypical follicular adenoma (FTA-aty) that are contained as inserted DNA in the recombinant vectors selected from the group of vectors having the following IMAGE clone ID No:
(iv-1) 208991, 136399, 503874, 123950, 1102600, 377560, 743367, 2048801, 42280, 813174, 30219, 1578721, 33133, 379517, 204686, 743331, 384404, 731598, 725345, 838611, 743982, 379540, 743619, 487848, 738916, 744074, 50491, 1089025, 666128, 740158, 341763, 740941, 729542, 251047, 742965, 140405, 111460, 724642, 723923, 714216, 731616, 309645, 366032, 740105, 744926, 364706, 739097, 1735081, 207920, 487436, 739193, 177857, 1089513, 743603, 2091389, 665784, 731422, 71428, 179534, 1012990, 666315, 1932202, 713831, 760065, 731380, 665632, 731202, 740604, 665086, 136686, 726763, 743270, 759865, 364741, 823590, 609631, 730926, 201467, 310519, 127360, 471641, 742977, 80384, 724238, 378813, 35628, 487103, 28584, 1075949, 731305, 503874, 741305, 208991, 743367, 380394, 123950, 23939, 714196, and 1102600,
(iv-2) 687667, 265558, 738970, 726830, 146976, 745490, 282587, 366388, 2074228, 669188, 668536, 366042, 208097, 743774, 713647, 134978, 731751, 300194, 713653, 724276, 301122, 730300, 669564, 668239, 738332, 713422, 594279, 1104732, 665376, 730938, 667174, 1877668, 300268, 587186, 711918, 72744, 1932168, 143759, 725791, 730971, 687468, 125552, 668815, 687782, 725454, 2074228, 146976, 2139315, 726830, 50202, 269873, 745490, and 265558,
(v) marker genes specific for microfollicular adenoma (FTA-b) that are contained as inserted DNA in the recombinant vectors selected from the group of vectors having the following IMAGE clone ID No:
(v-1) 740931, 740604, 172828, 725268, 136014, 731751, 594279, 731128, 379540, 725877, 743774, 136976, 178984, 208097, 627107, 209137, 741587, 236399, 234148, 129438, 23612, 234357, 1088781, 743118, 359461, 666128, 838611, 200018, 265558, 288807, 239086, 137454, 1735081, 742965, 160163, 1146624, 725707, 110938, 153411, 343987, 155345, 289337, 117072, 191881, 309697, 738315, 727496, 1056782, 486201, 665566, 119939, 25664, 1420842, 725758, 1129745, 713896, 175772, 345034, 364204, 669564, 743426, 1072914, 78294, 743259, 486204, 884365, 504539, 378813, 745018, 740158, 687667, 236386, 741661, 562904, 1641901, 741919, 1188588, 592598, 1870594, 114008, 809719, 2103105, 713422, 1705397, 1089025, 705147, 730037, 667144, 503874, 484577, 812955, 744606, 1016390, 487848, 251427, 1544175, 726147, 682207, 2364998, 731738, 725548, 1049033, 210587, 1131054, 744926, 724042, 743246, 743212, 131839, 823680, 740604, 487086, 730593, 591534, and 743118,
(v-2) 667892, 267358, 666007, 146976, 486510, 564994, 364436, 365271, 739193, 666315, 592276, 739097, 713653, 125552, 725364, 214008, 731404, 1087348, 296702, 251250, 469345, 669263, 727056, 744385, 731277, 512910, 34007, 744439, 365121, 230126, 811582, 366042, 33621, 730938, 665649, 668815, 740620, 502598, 726763, 364741, 208991, 136399, 25883, 136686, 383718, 364324, 724276, 726353, 382612, 295412, 645455, 501476, 739576, 143759, 740130, 31120, 78353, 731227, 26519, 364469, 813260, 342551, 809828, 753215, 364844, 564994, 740620, 267358, 667533, 666315, 666007, 1173570, 364569, 667514, and 487475,
(vi) marker genes specific for follicular carcinoma (FTC) that are contained as inserted DNA in the recombinant vectors selected from the group of vectors having the following IMAGE clone ID No:
(vi-1) 726684, 501476, 38775, 251250, 669318, 35356, 364469, 713653, 739193, 26519, 30219, 155345, 251452, 739953, 202897, 490710, 739097, 469345, 365121, 309009, 364436, 725345, 153411, 744897, 725758, 485803, 175268, 120649, 364777, 191881, 276658, 471574, 1129745, 742935, 343867, 713896, 236386, 276727, 342416, 124554, 724588, 1057773, 1420842, 666928, 727178, 1013595, 725548, 207920, 235882, 964736, 489326, 1146624, 1131054, 469647, 725696, 740941, 668815, 1705397, 687468, 195712, 342551, 743367, 80109, 1899942, 729489, 682451, 687551, 236055, 25077, 665327, 667250, 471834, 26519, and 365121,
(vi-2) 72744, 2467442, 726782, 743426, 136801, 135407, 135975, 137454, 726849, 238840, 324079, 486221, 669574, 266361, 134858, 731073, 359461, 744606, 731060, 666778, 726513, 236155, 744616, 744797, 23827, 743118, 609631, 143759, 665376, 742542, 725454, 135689, 713647, 743774, 136014, 724416, 687782, 723755, 726092, 731128, 782688, 80688, 53333, 22085, 46991, 486708, 730012, 236119, 549054, 2716748, 744797, 666784, 743043, 238840, 415554, and 729896,
(vii) marker genes specific for multinodular goitres (MNG) that are contained as inserted DNA in the recombinant vectors selected from the group of vectors having the following IMAGE clone ID No:
(vii-1) 811582, 188232, 682207, 356960, 236155, 501476, 773286, 173371, 276727, 30471, 645455, 745273, 744881, 1705397, 741474, 667527, 207920, 512910, 255398, 110101, 723923, 259552, 129438, 363955, 266361, 235882, 253386, 364885, 740105, 204381, 742837, 134615, 1116948, 136801, 1073288, 207920, 731292, 768246, 594510, and 745187,
(vii-2) 471641, 713422, 740941, 72744, 309645, 744505, 731726, 726782, 135689, 665668, 2055272, 151247, 366032, 172982, 743118, 364324, 745106, 172828, 740604, 744055, 154172, 131839, 53333, 744087, 726513, 295412, 134858, 30219, 739956, 109142, 486708, 78353, 725877, 726849, 782688, 345034, 25883, 2467442, 22711, 743774, 741954, 744606, 1420676, 724366, 1716286, 80688, 236399, 250883, 730829, 250463, 665668, 486356, 257773, 726782, 744505, 135689, 731726, 668625, 309645, and 740941,
(viii) marker genes specific for oncocytic adenoma (OTA) that are contained as inserted DNA in the recombinant vectors selected from the group of vectors having the following IMAGE clone ID No:
(viii-1) 80688, 666410, 725877, 729971, 236399, 726092, 724366, 172982, 1870594, 742100, 366388, 366042, 744983, 365349, 731275, 416959, 53333, 151247, 731726, 345034, 382612, 809719, 731738, 730012, 667348, 173820, 667313, 739247, 137454, 744087, 730300, 884365, 135689, 743774, 725707, 22085, 1860115, 1932168, 250883, 726849, 669564, 2266583, 139242, 740941, 729542, 364324, 665376, 665784, 51232, 731751, 175268, 723753, 609631, 238840, 669263, 252489, 486708, 666066, 730829, 33621, 23612, 667239, 740620, 731128, 739956, 725791, 726684, 486401, 23827, 365271, 365121, 134858, 782688, 741835, 590264, 471641, 738332, 379517, 726454, 726782, 731404, 2032639, 741954, 366115, 366289, 727154, 725268, 740130, 745106, 743125, 665668, 549054, 282587, 146976, 109142, 253386, 1101773, 131839, 214008, 669510, 665833, 162246, 743118, 738970, 310519, 742853, 726106, 30219, 682088, 724588, 745125, 364204, 172828, 666007, 727496, 740971, 823680, 1707527, 565317, 1641901, 309645, 666128, 1057773, 667174, 285693, 309009, 743259, 666778, 731152, 740347, 1896337, 665632, 379540, 757248, 134978, 1420676, 210587, 726424, 592598, 665881, 725511, 738896, 740885, 295412, 687782, 73703, 744756, 288807, 180259, 137131, 838611, 724042, 744366, 364412, 427786, 448163, 34007, 325008, 742977, 471101, 1256485, 730037, 742696, 713831, 725622, 486493, 744413, 724276, 725096, 234148, 239951, 134034, 868577, 713623, 366074, 149910, 1877668, 154172, 289337, 726513, 2306096, 726904, 364959, 727251, 744505, 667602, 342256, 1326169, 744055, 187482, 196115, 731060, 342416, 743237, 123950, 125552, 503874, 781738, 1654256, 743246, 740604, 739953, 744911, 364526, 503189, 738938, 208097, 25883, 324225, 245426, 723923, 248811, 738315, 743722, 1013595, 1737165, 727173, 744755, 140405, 300194, 744468, 2361911, 46991, 1352408, 1129745, 1089025, 745223, 713896, 725877, 530069, 724366, 666410, 729971, and 726849,
(viii-2) 258865, 485104, 430471, 730926, 731616, 365045, 485872, 724895, 179534, 486189, 731115, 364686, 731076, 743270, 178255, 33133, 366820, 365056, 666334, 666703, 1086823, 364741, 666794, 137478, 177857, 42280, 31924, 235938, 2484270, 213529, 724014, 2048801, 731380, 743331, 730814, 1174287, 667252, 731598, 136863, 744895, 382738, 667110, 471574, 23876, 760065, 1085884, 745072, 2384812, 724238, 731422, 744899, 666279, 341763, 731305, 665433, 740105, 236119, 30471, 384844, 665086, 364885, 383868, 364717, 714498, 744074, 512910, 382621, 665649, 379937, 364706, 743982, 743619, 587186, 1705397, 741474, 241481, 2030501, 433155, 230126, 35628, 363955, 1012990, 71428, 525221, 731202, 667527, 364729, 743603, 173371, 753215, 471664, 742837, 668186, 687468, 381854, 738916, 1075949, 201467, 487152, 767779, 152847, 40304, 740457, 486221, 382791, 591907, 566862, 364777, 744616, 742935, 724642, 257555, 1049033, 667258, 382521, 731021, 384404, 742061, 382069, 731277, 727289, 173200, 727178, 795736, 177967, 668452, 823590, 382002, 726821, 813174, 725231, 731742, 731227, 1917430, 667355, 135630, 383718, 487848, 729875, 813654, 740158, 1624260, 773286, 284828, 729929, 731073, 731023, 136686, 472160, 484577, 486304, 740931, 377560, 25664, 669557, 811024, 1285305, 502658, 276727, 236155, 666928, 1103402, 682585, 966894, 134615, 487436, 296702, 1578721, 485803, 1102600, 364469, 739576, 742542, 2074228, 743517, 340657, 156183, 742763, 485085, 80384, 266361, 40633, 175772, 666315, 744797, and 236331,
(ix) marker genes specific for atypical oncocytic adenoma (OTA-aty) that are contained as inserted DNA in the recombinant vectors selected from the group of vectors having the following IMAGE clone ID No:
(ix-1) 26519, 172982, 744439, 745003, 238840, 1420676, 742935, 503189, 151247, 214008, 365121, 730300, 25883, 545475, 738332, 669263, 125552, 744087, 740620, 366042, 884365, 78353, 109142, 742100, 590264, 379517, 667313, 813654, 730037, 135689, 740130, 742935, 1855110, 796513, 376462, 230882, 813654, and 136862,
(ix-2) 487152, 740457, 667527, 382002, 382791, 2384812, 566862, 1075949, 730814, 235938, 745072, 341763, 383718, 486304, 379937, 666279, 241481, 731380, 743270, 1917430, 682585, 666703, 1049033, 1085884, 724642, 665086, 742061, 767779, 729875, 724014, 743603, 714498, 731277, 727289, 744895, 35628, 173200, 743619, 726821, 284828, 129438, 731616, 487103, 667355, 1012990, 487848, 471664, 744899, 42280, 713623, 667110, 502658, 730926, 484577, 1174287, 236155, 381854, 365056, 2030501, 731021, 177967, 667258, 525221, 743331, 135407, 738916, 669557, 382521, 731305, 730401, 713623, 235938, 669098, 1049033, 730814, 382002, 667527, 740457, 382791, and 665086,
(x) marker genes specific for oncocytic carcinoma (OTC) that are contained as inserted DNA in the recombinant vectors selected from the group of vectors having the following IMAGE clone ID No:
(x-1) 782688, 78353, 666410, 740130, 745003, 740620, 731726, 1716286, 250883, 1420676, 740941, 135689, 744087, 724366, 109142, 2055272, 486493, 590264, 238840, 713623, 469345, 80688, 738970, 725791, 364412, 366042, 146976, 739955, 1844968, 365271, 187482, 668815, 744606, 501476, 180259, 1101773, 809828, 731404, 366115, 726353, 713422, 173820, 137454, 665668, 2032639, 366289, 726763, 549054, 743367, 135941, 131839, 46991, 724416, 282587, 503874, 744983, 209137, 502598, 667348, 812143, 745106, 730012, 726092, 739193, 726684, 175268, 740347, 471101, 151247, 365121, 345034, 252489, 364324, 565317, 34007, 162246, 253386, 731060, 738896, 743125, 743774, 137720, 739956, 741954, 139242, 739097, 309645, 73703, 342256, 609631, 667313, 51232, 745490, 740193, 469647, 486401, 22711, 484504, 2349125, 665376, 724588, 154172, 267358, 739953, 838611, 427786, 1129745, 725758, 730300, 1089025, 729542, 1932168, 687667, 309697, 471641, 487130, 731738, 545475, 160163, 365349, 239086, 665982, 366388, 25883, 78353, 359454, 666410, 740620, 740347, and 364412,
(x-2) 40304, 382423, 173371, 1917430, 745072, 727289, 486221, 740457, 742061, 512910, 487848, 742542, 258966, 723755, 502658, 471664, 1012990, 729875, 25664, 487152, 724596, 731073, 666928, 668452, 486304, 382002, 1285305, 235938, 484577, 341763, 382791, 241481, 742837, 258865, 767779, 566862, 725454, 740105, 731021, 813654, 1174287, 71428, 1049033, 382738, 665433, 823590, 665086, 1645668, 645455, 668239, 741474, 364885, 236119, 35628, 682585, 1075949, 773286, 382069, 213529, 668536, 257555, 42280, 31924, 731380, 725231, 727251, 726821, 236155, 740158, 760065, 587186, 40633, 486189, 731742, 266361, 1085884, 152847, 669188, 363955, 724014, 813174, 284828, 134615, 255398, 343987, 731115, 730814, 667252, 740931, 743603, 174396, 364729, 1103402, 727178, 744899, 2048801, 382521, 811024, 39593, 813654, 666928, 175080, 667991, 136933, 723755, 173371, 742061, 471664, and 1917430,
(xi) marker genes specific for papillary carcinoma (PTC) that are contained as inserted DNA in the recombinant vectors selected from the group of vectors having the following IMAGE clone ID No:
(xi-1) 1624260, 743517, 28584, 296702, 80384, 669557, 731227, 795736, 787938, 384844, 384404, 502598, 1086823, 382621, 1716286, 823590, 731422, 23876, 25883, 667258, 364729, 433155, 669318, 738916, 727289, 731598, 739097, 383718, 364777, 667527, 366032, 744074, 666315, 724596, 724014, 742935, 1102600, 743982, 744616, 724642, 726763, 30471, 726513, 2384812, 309697, 740457, 730926, 666007, 382069, 135941, 742061, 731616, 78353, 813174, 485104, 2484270, 744439, 724238, 740158, 382791, 208991, 731305, 35628, 179534, 731021, 364324, 726821, 666703, 665668, 365045, 668452, 809828, 382738, 201467, 363955, 730814, 739193, 731277, 744797, 366820, 383868, 682585, 382002, 745072, 743270, 742837, 40304, 1917430, 813260, 744055, 379937, 136686, 1012990, 1285305, 486304, 665433, 742542, 731202, 1075949, 668815, 744895, 2048801, 744385, 42280, 667110, 31924, 284828, 364706, 177967, 71428, 364686, 730829, 469647, 753215, 33133, 724225, 738938, 430471, 235938, 767779, 731380, 1578721, 2467442, and 1624260,
(xi-2) 359051, 343987, 301122, 594279, 745490, 711918, 687667, 378813, 265558, 162246, 812955, 145105, 757248, 324079, 741587, 740347, 33470, 208097, 743125, 364526, 136976, 188393, 1641901, 140405, 627107, 730012, 725511, 359461, 726106, 744881, 486204, 668239, 823680, 725707, 356841, 251427, 236331, 1877668, 134978, 309009, 376153, 234148, 731128, 731047, 727251, 1896337, 38775, 743367, 2306096, 23612, 125552, 743774, 236399, 2266583, 682088, 966894, 175772, 282587, 738970, 251452, 738332, 812143, 1103402, 2113771, 868577, 731738, 549054, 713647, 379540, 235882, 1932168, 725096, 472114, 731060, 128065, 1860115, 33285, 135689, 266361, 137454, 730300, 136014, 742100, 731751, 416959, 209137, 22711, 1104732, 300268, 669564, 730037, 725877, 705147, 364204, 366388, 726830, 119939, 110101, 364959, 486201, 253386, 665376, 364412, 188232, 725454, 726092, 809719, 741835, 135975, 345034, 1337232, 724366, 136801, 669510, 366042, 134858, 727251, 627107, 376153, 743125, 140405, 823680, 136976, 324079, 378813, 687667, and 265558,

In certain embodiments, if step b) of the method is performed by quantifying the expression level of less than 20 disease-specific marker genes in each group of disease-specific marker genes, among groups (i) to (xi), then the said disease-specific marker genes may be selected; in each of groups (i) to (xi), from the groups consisting of:
(i) marker genes specific for autoimmune thyroiditis (AT) that are contained as inserted DNA in the recombinant vectors selected from the group of vectors having the following IMAGE clone ID No:
(i-1) 669510, 725096, 366100, 325155, 154172, 787938, 744055, 592276, 740941, 73778, 667174, 739193, 295412, 23827, 136686, 744385, 471641, 724276, 28584 and 268972,
(i-2) 43541, 687990, 365990, 562904, 725548, 365877, 742763, 180259, 2449149, 1089025, 713623, 382760, 258747, 743961, 174396, 724306, 1505308, 726454, 665452 and 665833,
(ii) marker genes specific for Grave's disease (GD that are contained as inserted DNA in the recombinant vectors selected from the group of vectors having the following IMAGE clone ID No:
(ii-1) 25664, 868169, 731380, 1049033, 730942, 731616, 726578, 366388, 485872, 1662274, 2074228, 730926, 745106, 731021, 207920, 258865, 503189, 745490, 1103402 and 1911930,
(ii-2) 687990, 743961, 486493, 379279 and 667313,
(iii) marker genes specific for macrofollicular adenoma (FTA-a) that are contained as inserted DNA in the recombinant vectors selected from the group of vectors having the following IMAGE clone ID No:
(iii-1) 2348237, 135379, 743851, 1712992, 376699, 1251197, 1174287, 173841, 486221, 265345, 666564, 252382, 173371, 731073, 253386, 1285305, 342700, 179214, 3054031 and 359797,
(iii-2) 745003, 1844968, 364324, 357201, 24392, 1420676, 687532, 724562, 729510, 725371, 24083, 738938, 730938, 148796, 137205, 174683, 21808, 3659591, 325155 and 666792,
(iv) marker genes specific for atypical follicular adenoma (FTA-aty) that are contained as inserted DNA in the recombinant vectors selected from the group of vectors having the following IMAGE clone ID No:
(iv-1) 503874, 666128, 838611, 208991, 739097, 1089025, 725548, 739193, 155839, 28584, 202897, 743331, 743619, 725345, 345034, 235938, 729924, 681957, 30219 and 773330,
(iv-2) 50202, 738970, 128694, 741891, 666671, 366388, 146976, 687667, 587186, 667727, 669136, 744001, 666361, 668300, 713647, 2074228, 282587, 179288, 687270 and 726830,
(v) marker genes specific for microfollicular adenoma (FTA-b) that are contained as inserted DNA in the recombinant vectors selected from the group of vectors having the following IMAGE clone ID No:
(v-1) 740931; 731751; 840687; 666128; 379540; 667259; 725585; 503874; 23612; 3054031; 208097; 265558; 136976, 838611, 725707, 594279, 343987, 487474, 731128, 279172 and 773330,
(v-2) 724609, 365913, 666154, 267358, 665379, 667892, 251250, 713653, 365589, 1087348, 366042, 743146, 174654, 744657, 214008, 666007, 137005, 364436, 738558 and 146976,
(vi) marker genes specific for follicular carcinoma (FTC) that are contained as inserted DNA in the recombinant vectors selected from the group of vectors having the following IMAGE clone ID No:
(vi-1) 501476, 309009, 739193, 251452, 681957, 739097, 364448, 469549, 202897, 195340, 731380, 2345206, 23765, 471641, 502244, 713653, 486189, 740604, 682207 and 740941,
(vi-2) 782688, 136014, 135980, 725335, 724150, 307094, 723752, 510318, 726578, 324079, 2139164, 143759, 739237, 152655, 136646, 309697, 384087, 365408, 376699 and 134671
(vii) marker genes specific for multinodular goitres (MNG) that are contained as inserted DNA in the recombinant vectors selected from the group of vectors having the following IMAGE clone ID No:
(vii-1) 284828, 173841, 667527, 202897, 469549, 207920, 469647, 363955, 323438, 276658, 263251, 731277, 35628, 741474, 743394, 212366, 258865, 724014, 742837 and 1117183
(vii-2) 687625, 726782, 364324, 148796, 744505, 26164, 665668, 713837, 172828, 731726, 151247, 486356, 342181, 471641, 72744, 135689, 740941, 366032 and 154172,

(viii) marker genes specific for oncocytic adenoma (OTA) that are contained as inserted DNA in the recombinant vectors selected from the group of vectors having the following IMAGE clone ID No:
(viii-1) 80688, 1870594, 731738, 1870594, 365348, 681875, 731751, 136114, 365161, 491004, 724366, 744983, 740718, 884365, 687381, 236399, 731726, 809719, 757248 and 172982,
(viii-2) 1662274, 145696, 730926, 731115, 155345, 376356, 666794, 739109, 179534, 628418, 741406, 485872, 232388, 667110, 724895, 33133, 730834, 743270, 731380, and 713913,
(ix) marker genes specific for atypical oncocytic adenoma (OTA-aty) that are contained as inserted DNA in the recombinant vectors selected from the group of vectors having the following IMAGE clone ID No:
(ix-1) 745003, 740130, 471641, 682088, 731404, 2254555, 2381770, 214008, 300268, 2467442, 125552, 666517, 366159, 740927, 665784, 24392, 727457, 148796, 590264 and 665632,
(ix-2) 724556, 668329, 173200, 174654, 731227, 1624260, 743078, 739576, 3054031 and 119772,
(x) marker genes specific for oncocytic varcinoma (OTC) that are contained as inserted DNA in the recombinant vectors selected from the group of vectors having the following IMAGE clone ID No:
(x-1) 727123, 724366, 366289, 471641, 501476, 744943, 2237263, 155839, 140347, 743774, 1716286, 365271, 236399, 731423, 252291, 812967, 739097, 471702, 1420676 and 739193,
(x-2) 731019, 366085, 258127, 382002, 714414, 486189, 725188, 1241974, 666703, 487152, 40304, 811024, 25865, 666279, 745133, 347362, 258966, 471664, 82687 and 740931,
(xi) marker genes specific for papillary carcinoma (PTC) that are contained as inserted DNA in the recombinant vectors selected from the group of vectors having the following IMAGE clone ID No:
(xi-1) 726763, 739193, 28584, 739097, 25883, 1716286, 365045, 666367, 379200, 365271, 731227, 726760, 384404, 208991, 744943, 1846326, 364729, 730926, 795736 and 136686,
(xi-2) 758037, 1932168, 489945, 738970, 667756, 265558, 2113771, 365562, 1712992, 739237, 135539, 159809, 268972, 135303, 1916307, 356841, 682088, 665437, 744945 and 2728204,

In certain other embodiments, if step b) of the method is performed by quantifying the expression level of less than 20 disease-specific marker genes in each group of disease-specific marker genes, among groups (i) to (xi), then the said disease-specific marker genes may be selected; in each of groups (i) to (xi), from the groups consisting of:
(i) marker genes specific for autoimmune thyroiditis (AT) that are contained as inserted DNA in the recombinant vectors selected from the group of vectors having the following IMAGE clone ID No:
(i-1) 1837488, 284828, 729542, and 238705,
(i-2) 52740, 208097, 36371, 740617, 1086914, 2108870, 665651, 1844689, 448163, 1929371, 1352408, 742649, 1056782, 668454, 43629, and 155717,
(ii) marker genes specific for Grave's disease (GD that are contained as inserted DNA in the recombinant vectors selected from the group of vectors having the following IMAGE clone ID No:
(ii-1) 23817, 24623, 724596, and 180851,
(ii-2) 135773, 136686, 731711, 250771, 122063, 744385, 740140, 262894, 53081, 258698, 53119, 136366, 665674, 726424, 178161, and 729489,
(iii) marker genes specific for macrofollicular adenoma (FTA-a) that are contained as inserted DNA in the recombinant vectors selected from the group of vectors having the following IMAGE clone ID No:
(iii-1) 1174287, 487379, and 24065,
(iii-2) 745465, 173820, 24392, 300194, 208991, 24532, 725371, 745003, 738938, 739955, 22711, 376003, 1844968, 359202, 295412, 1420676, and 25883,
(iv) marker genes specific for atypical follicular adenoma (FTA-aty) that are contained as inserted DNA in the recombinant vectors selected from the group of vectors having the following IMAGE clone ID No:
(iv-1) 503874, 741305, 208991, 136399, 743367, 380394, 123950, 23939, 714196, and 1102600,
(iv-2) 2074228, 146976, 738970, 2139315, 726830, 50202, 269873, 745490, 265558, and 687667,
(v) marker genes specific for microfollicular adenoma (FTA-b) that are contained as inserted DNA in the recombinant vectors selected from the group of vectors having the following IMAGE clone ID No:
(v-1) 740931, 740604, 487086, 172828, 730593, 591534, and 743118,
(v-2) 364844, 564994, 740620, 267358, 667533, 666315, 146976, 666007, 667892, 1173570, 364569, 667514, and 487475,
(vi) marker genes specific for follicular carcinoma (FTC) that are contained as inserted DNA in the recombinant vectors selected from the group of vectors having the following IMAGE clone ID No:
(vi-1) 726684, 682451, 687551, 236055, 25077, 665327, 667250, 471834, 26519, and 365121,
(vi-2) 2716748, 744797, 666784, 743043, 238840, 415554, 72744, 729896, 743426, and 2467442,
(vii) marker genes specific for multinodular goitres (MNG) that are contained as inserted DNA in the recombinant vectors selected from the group of vectors having the following IMAGE clone ID No:
(vii-1) 1073288, 207920, 731292, 768246, 594510, and 745187,
(vii-2) 250463, 665668, 486356, 713422, 257773, 726782, 744505, 72744, 135689, 731726, 668625, 309645, 740941, and 471641,
(viii) marker genes specific for oncocytic adenoma (OTA) that are contained as inserted DNA in the recombinant vectors selected from the group of vectors having the following IMAGE clone ID No:
(viii-1) 725877, 530069, 724366, 666410, 729971, 80688, and 726849,
(viii-2) 365934, 753215, 730926, 485872, 365045, 724895, 179534, 486189, 53341, 485104, 731616, 430471, and 258865,
(ix) marker genes specific for atypical oncocytic adenoma (OTA-aty) that are contained as inserted DNA in the recombinant vectors selected from the group of vectors having the following IMAGE clone ID No:
(ix-1) 26519, 742935, 1855110, 796513, 376462, 230882, 813654, and 136862,
(ix-2) 730401, 713623, 235938, 669098, 1049033, 487152, 730814, 382002, 667527, 740457, 382791, and 665086,
(x) marker genes specific for oncocytic varcinoma (OTC) that are contained as inserted DNA in the recombinant vectors selected from the group of vectors having the following IMAGE clone ID No:
(x-1) 78353, 782688, 359454, 666410, 740620, 740347, and 364412,
(x-2) 39593, 813654, 666928, 175080, 667991, 136933, 723755, 173371, 742061, 471664, 40304, 382423, and 1917430,
(xi) marker genes specific for papillary carcinoma (PTC) that are contained as inserted DNA in the recombinant vectors selected from the group of vectors having the following IMAGE clone ID No:
(xi-1) 1624260,
(xi-2) 729542, 727251, 627107, 376153, 743125, 140405, 823680, 136976, 324079, 378813, 711918, 208097, 687667, 359051, 343987, 745490, 265558, 301122, and 594279,

In certain most preferred embodiments, if step b) of the method is performed by quantifying the expression level of highly significant disease-specific marker genes which are selected, in each of groups (i) to (xi), from the groups consisting of:
(i) marker genes specific for autoimmune thyroiditis (AT) that are contained as inserted DNA in the recombinant vectors selected from the group of vectors having the following IMAGE clone ID No:
(i-2) 1088781 (SEQ ID No 3), 1089513 (SEQ ID No 4), 1131054 (SEQ ID No 5), 116906 (SEQ ID No 6-7), 122874 (SEQ ID No 8-9), 123950 (SEQ ID No 10-11), 1251197 (SEQ ID No 12), 1256485 (SEQ ID No 13), 1286238 (SEQ ID No 14), 1326169 (SEQ ID No 15), 1352408 (SEQ ID No 16), 140405 (SEQ ID No 17-18), 1578721 (SEQ ID No 19), 162246 (SEQ ID No 20-21), 1705397 (SEQ ID No 22), 1707527 (SEQ ID No 23), 174396 (SEQ ID No 24-25), 180259 (SEQ ID No 26-27), 1870594 (SEQ ID No 28), 188393 (SEQ ID No 29-30), 1942634 (SEQ ID No 31), 201467 (SEQ ID No 32-33), 204686 (SEQ ID No 34-35), 210587 (SEQ ID No 36-37), 2238108 (SEQ ID No 38), 245426 (SEQ ID No 39-40), 250883 (SEQ ID No 41-42), 2728204 (SEQ ID No 43), 279470 (SEQ ID No 44), 287843 SEQ ID No 45), 3054031 (SEQ ID No 46-47), 310519 (SEQ ID No 48-49), 342256 (SEQ ID No 50-51), 345034 (SEQ ID No 52-53), 356841 (SEQ ID No 54-55), 356960 (SEQ ID No 56-57), 366289 (SEQ ID No 58), 379200 (SEQ ID No 59), 382760 (SEQ ID No 60-61), 427786 (SEQ ID No 62-63), 448163 (SEQ ID No 64), 46991 (SEQ ID No 65-66), 485803 (SEQ ID No 67-68), 486401 (SEQ ID No 69-70), 489326 SEQ ID No 71-72), 503874 (SEQ ID No 73-74), 50491 (SEQ ID No 75), 590264 (SEQ ID No 76-77), 592598 (SEQ ID No 78-79), 609631 (SEQ ID No 80-81), 665452 (SEQ ID No 82-83), 666410 (SEQ ID No 84), 667239 (SEQ ID No 85), 668584 (SEQ ID No 86), 682207 (SEQ ID No 87-88), 687990 (SEQ ID No 89-90), 713623 (SEQ ID No 91-92), 725473 (SEQ ID No 93-94), 725548 (SEQ ID No 95-96), 726454 (SEQ ID No 97-98), 727496 (SEQ ID No 99-100), 730037 (SEQ ID No 101-102), 731751 (SEQ ID 107, 738944 (SEQ ID No 108), 740347 (SEQ ID No 109-110), 740931 (SEQ ID No 111-112), 742696 (SEQ ID No 113-114), 742853 (SEQ ID No 115-116), 743246 (SEQ ID No 117-118), 743367 (SEQ ID No 119-120), 744911 (SEQ ID No 121), 80688 (SEQ ID No 122), 811024 (SEQ ID No 123-124), 813174 (SEQ ID No 125-126), 823680 (SEQ ID No 127-128) and 884365 (SEQ ID No 129),
(i-1) 1174287 (SEQ ID No 130), 119772 (SEQ ID No 131-132), 133531 (SEQ ID No 133-134), 135689 (SEQ ID No 135-136), 136919 (SEQ ID No 137-138), 139242 (SEQ ID No 139-140), 1420676 (SEQ ID No 141), 148796 (SEQ ID No 142), 154172 (SEQ ID No 143), 155345 (SEQ ID No 144-145), 1716286 (SEQ ID No 146), 172982 (SEQ ID No 147), 173841 (SEQ ID No 148-149), 1846326 (SEQ ID No 150), 1877668 (SEQ ID No 151), 208991 (SEQ ID No 152-153), 214008 (SEQ ID No 154), 2266583 (SEQ ID No 155), 22711 (SEQ ID No 156), 2384812 (SEQ ID No 157), 258127 (SEQ ID No 158), 25883 (SEQ ID No 159-160), 258865 (SEQ ID No 161), 265558 (SEQ ID No 162-163), 28584 (164-165), 295412 (166-167), 301122 (SEQ ID No 168), 309645 (SEQ ID No 169-170), 324079 (SEQ ID No 171-172), 325155 (SEQ ID No 173-174), 343987 (SEQ ID No 175), 359051 (SEQ ID No 176-177), 359461 (SEQ ID No 178-179), 364324 (SEQ ID No 180-181), 364412 (SEQ ID No 182-183), 365045 (SEQ ID No 184-185), 365121 (SEQ ID No 186-187), 365271 (SEQ ID No 188-189), 365589 (SEQ ID No 190-191), 365913 (SEQ ID No 192), 366032 (SEQ ID No 193-194), 366042 (SEQ ID No 195-196), 366100 (SEQ ID No 197-198), 38403 (SEQ ID No 199), 471641 (SEQ ID No 200-201), 471664 (SEQ ID No 202-203), 487152 (SEQ ID No 204-205), 512910 (SEQ ID No 206-207), 545475 (SEQ ID No 208-209), 549054 (SEQ ID No 210-211), 587186 (SEQ ID No 212-213), 592276 (SEQ ID No 214-215), 628418 (SEQ ID No 216-217), 665086 (SEQ ID No 218-219), 665376 SEQ ID No 220-221), 666154 (SEQ ID No 222-223), 666361 (SEQ ID No 224-225), 667174 (SEQ ID No 226), 667348 (SEQ ID No 227-228), 667892 (SEQ ID No 229), 668684 (SEQ ID No 230), 681875 (SEQ ID No 231-232), 687667 (SEQ ID No 233), 714437 (SEQ ID No 234-235), 724562 (SEQ ID No 236), 724892 (SEQ ID No 237-238), 725076 (SEQ ID No 239-240), 726596 (SEQ ID No 241-242), 727056 (SEQ ID No 243-244), 727251 (SEQ ID No 245), 727289 (SEQ ID No 246-247), 72744 (SEQ ID No 248-249), 729510 (SEQ ID No 250-251), 730012 (SEQ ID No 252-253), 730300 (SEQ ID No 254), 731060 (SEQ ID No 255-256), 731115 (SEQ ID No 257-258), 731726 (SEQ ID No 259-260), 738332 (SEQ ID No 261-262), 738970 (SEQ ID No 263), 739097 (SEQ ID No 264-265), 739193 (SEQ ID No 266-267), 740457 (SEQ ID No 268-269), 740941 (SEQ ID No 270), 743774 (SEQ ID No 271), 744087 (SEQ ID No 272), 744439 (SEQ ID No 273), 745003 (SEQ ID No 274), 745072 (SEQ ID No 275), 745490 (SEQ ID No 276), 782688 (SEQ ID No 277-278) and 78353 (SEQ ID No 279-280), 787938 (SEQ ID No 281),
(ii) marker genes specific for Grave's disease (GD that are contained as inserted DNA in the recombinant vectors selected from the group of vectors having the following IMAGE clone ID No:
(ii-1) 145696 (SEQ ID No 282), 151247 (SEQ ID No 283-284), 1662274 (SEQ ID No 285), 173296 (SEQ ID No 286-287), 341763 (SEQ ID No 288-289), 486189 (SEQ ID No 290-291), 594279 (SEQ ID No 292), 730926 (SEQ ID No 293-294), 731616 (SEQ ID No 295) and 740620 (SEQ ID No 296-297),
(iii) marker genes specific for macrofollicular adenoma (FTA-a) that are contained as inserted DNA in the recombinant vectors selected from the group of vectors having the following IMAGE clone ID No:
(iii-2) 136114 (SEQ ID No 298-299), 364575 (SEQ ID No 300-301) and 366436 (SEQ ID No 302-303), [Over-expressed]
(iii-1) 1188588 (SEQ ID No 304-305), 137131 (SEQ ID No 306-307), 137454 (SEQ ID No 308), 173371 (SEQ ID No 309-310), 235882 (SEQ ID No 311-312), 235938 (SEQ ID No 313-314), 253386 (SEQ ID No 315-316), 265345 (SEQ ID No 317-318), 276727 (SEQ ID No 319), 42280 (SEQ ID No 320-321), 486221 (SEQ ID No 322-323), 666703 (SEQ ID No 324-325), 682276 (SEQ ID No 326), 725707 (SEQ ID No 327-328), 726600 (SEQ ID No 329-330), 741406 (SEQ ID No 331-332), 743259 (SEQ ID No 333-334), 744983 (SEQ ID No 335) and 838611 (SEQ ID No 336-337),
(iv) marker genes specific for atypical follicular adenoma (FTA-aty) that are contained as inserted DNA in the recombinant vectors selected from the group of vectors having the following IMAGE clone ID No:
(iv-2) 208097 (SEQ ID No 338-339), 669136 (SEQ ID No 340-341) and 687468 (SEQ ID No 342-343),
(iv-1) 1089025 (SEQ ID No 344), 1536698 (SEQ ID No 345), 155839 (SEQ ID No 346-347), 238840 (SEQ ID No 348-349), 30219 (SEQ ID No 350-351), 33133 (SEQ ID No 352-353), 378813 (SEQ ID No 354), 379517 (SEQ ID No 355), 379540 (SEQ ID No 356), 40304 (SEQ ID No 357), 665784 (SEQ ID No 358-359), 666128 (SEQ ID No 360-361), 667527 (SEQ ID No 362), 724014 SEQ ID No 363-364), 739109 (SEQ ID No 365), 740105 (SEQ ID No 366), 740604 (SEQ ID No 367-368) and 743270 (SEQ ID No 369-370),
(v) marker genes specific for microfollicular adenoma (FTA-b) that are contained as inserted DNA in the recombinant vectors selected from the group of vectors having the following IMAGE clone ID No:
(v-1) 1641901 (SEQ ID No 371), 23612 (SEQ ID No 372-373), 236399 (SEQ ID No 374-375), 491004 (SEQ ID No 376-377), 667365 (SEQ ID No 378-379), 724366 (SEQ ID No 380-381), 725877 (SEQ ID No 382-383), 731128 (SEQ ID No 384-385), 731738 (SEQ ID No 386-387) and 809719 (SEQ ID No 388-389),
(vi) marker genes specific for follicular carcinoma (FTC) that are contained as inserted DNA in the recombinant vectors selected from the group of vectors having the following IMAGE clone ID No:
(vi-1) 179334 (SEQ ID No 390-391), 713671 (SEQ ID No 392-393), 726684 (SEQ ID No 394-395) and 757248 (SEQ ID No 396)
(vii) marker genes specific for multinodular goitres (MNG) that are contained as inserted DNA in the recombinant vectors selected from the group of vectors having the following IMAGE clone ID No:
(vii-2) 194302 (SEQ ID No 397-398),
(vii-1) 1012990 (SEQ ID No 399), 208439 (SEQ ID No 400-401) and 36521 (SEQ ID No 402-403)
(viii) marker genes specific for oncocytic adenoma (OTA) that are contained as inserted DNA in the recombinant vectors selected from the group of vectors having the following IMAGE clone ID No:
(viii-2) 430471 (SEQ ID No 404)
(viii-1) 112819 (SEQ ID No 405), 180082 (SEQ ID No 406-407), 365348 (SEQ ID No 408-409), 366115 (SEQ ID No 410-411), 667310 (SEQ ID No 412-413), 713831 (SEQ ID No 414-415), 726092 (SEQ ID No 416-417), 730606 (SEQ ID No 418-419) and 743125 (SEQ ID No 420)

The sequence references (SEQ IDs) consist of nucleotide sequences that are comprised in the corresponding disease-specific marker genes. For each disease-specific marker gene that is listed above, a reference to (i) the 3′-end sequence, (ii) the 5′-end sequence or (iii) both the 3′-end sequence and the 5′-end sequence is given in parentheses.

Indeed, the sequences that are referred to above may be used as such as nucleic acid primers or probes specific for a given disease-specific marker gene. These sequences may also be used to design other nucleic acid probes or primers specific for a given disease-specific marker gene.

These sequences may also be used to amplify the nucleic acid insert of the corresponding IMAGE clone vector, so as to generate a suitable amount of a given disease-specific marker gene nucleic acid.

In certain other most preferred embodiments, if step b) of the method is performed by quantifying the expression level of highly significant disease-specific marker genes which are selected, in each of groups (i) to (xi), from the groups consisting of:
(i) marker genes specific for autoimmune thyroiditis (AT) that are contained as inserted DNA in the recombinant vectors selected from the group of vectors having the following IMAGE clone ID No:
(i-2) 36371 (SEQ ID No 421-422), 448163 (SEQ ID No 64), 740617 (SEQ ID No 423-424), 52740 (SEQ ID No 425-426), 208097 (SEQ ID No 338-339), 1086914 (SEQ ID No 427), 1929371 (SEQ ID No 428), and 742649 (SEQ ID No 429),
(i-1) 729542 (SEQ ID No 430-431), 1837488 (SEQ ID No 432), 284828 (SEQ ID No 433), and 238705 (SEQ ID No 434),
(ii) marker genes specific for Grave's disease (GD that are contained as inserted DNA in the recombinant vectors selected from the group of vectors having the following IMAGE clone ID No:
(ii-2) 136366 (SEQ ID No 435-436), 726424 (SEQ ID No 437-438), 178161 SEQ ID No 439-440), and 665674 (SEQ ID No 441-442),
(ii-1) 23817 (SEQ ID No 443),
(iii) marker genes specific for macrofollicular adenoma (FTA-a) that are contained as inserted DNA in the recombinant vectors selected from the group of vectors having the following IMAGE clone ID No:
(iii-2) 1420676 (SEQ ID No 141), 739955 (SEQ ID No 444-445), 745003 (SEQ ID No 274), and 738938 (SEQ ID No 446),
(iv) marker genes specific for atypical follicular adenoma (FTA-aty) that are contained as inserted DNA in the recombinant vectors selected from the group of vectors having the following IMAGE clone ID No:
(iv-2) 687667 (SEQ ID No 233), and 738970 (SEQ ID No 263),
(iv-1) 136399 (SEQ ID No 447-448), and 380394 (SEQ ID No 449-450),
(v) marker genes specific for microfollicular adenoma (FTA-b) that are contained as inserted DNA in the recombinant vectors selected from the group of vectors having the following IMAGE clone ID No:
(v-2) 364569 (SEQ ID No 451), 487475 (SEQ ID No 452-453), 667892 (SEQ ID No 229), 364844 (SEQ ID No 454-455), and 146976 (SEQ ID No 456-457),
(v-1) 740931 (SEQ ID No 111-112), and 172828 (SEQ ID No 458-459),
(vi) marker genes specific for follicular carcinoma (FTC) that are contained as inserted DNA in the recombinant vectors selected from the group of vectors having the following IMAGE clone ID No:
(vi-2) 743426 (SEQ ID No 460), 72744 (SEQ ID No 248-249), 729896 (SEQ ID No 461-462), and 2467442 (SEQ ID No 463),
(vi-1) 667250 (SEQ ID No 464-465), 687551 (SEQ ID No 466), 726684 (SEQ ID No 394-395), 665327 (SEQ ID No 467-468), 236055 (SEQ ID No 469), 471834 (SEQ ID No 470-471), and 25077 (SEQ ID No 472-473),
(vii) marker genes specific for multinodular goitres (MNG) that are contained as inserted DNA in the recombinant vectors selected from the group of vectors having the following IMAGE clone ID No:
(vii-2) 668625 (SEQ ID No 474), 471641 (SEQ ID No 200-201), 713422 (SEQ ID No 475), 72744 (SEQ ID No 248-249), and 486356 (SEQ ID No 476-477),
(viii) marker genes specific for oncocytic adenoma (OTA) that are contained as inserted DNA in the recombinant vectors selected from the group of vectors having the following IMAGE clone ID No:
(viii-2) 486189 (SEQ ID No 290-291), 258865 (SEQ ID No 161), and 485104 (SEQ ID No 478),
(viii-1) 80688 (SEQ ID No 122),
(ix) marker genes specific for oncocytic adenoma (OTA-aty) that are contained as inserted DNA in the recombinant vectors selected from the group of vectors having the following IMAGE clone ID No:
(ix-2) 487152 (SEQ ID No 204-205),
(ix-1) 26519 (SEQ ID No 479-480), 1855110 (SEQ ID No 481), and 796513 (SEQ ID No 482),
(x) marker genes specific for oncocytic adenoma (OTC) that are contained as inserted DNA in the recombinant vectors selected from the group of vectors having the following IMAGE clone ID No:
(x-2) 40304 (SEQ ID No 357), and 382423 (SEQ ID No 483),
(x-1) 782688 (SEQ ID No 277-278),and
(xi) marker genes specific for oncocytic adenoma (PTC) that are contained as inserted DNA in the recombinant vectors selected from the group of vectors having the following IMAGE clone ID No:
(xi-2) 359051 (SEQ ID No 176-177), 343987 (SEQ ID No 175), 594279 (SEQ ID No 292), 729542 (SEQ ID No 430-431), 208097 (SEQ ID No 338-339), 301122 (SEQ ID No 168), 745490 (SEQ ID No 276), and 711918 (SEQ ID No 484).

In some embodiments, it must be also used marker genes for healthy thyroid that are contained as inserted DNA in the recombinant vectors selected from the group of vectors having the following IMAGE clone ID No:
137131 (SEQ ID No 306-307), 238840 (SEQ ID No 348-349), 284828 (SEQ ID No 433), 310519 (SEQ ID No 48-49), 448163 (SEQ ID No 64), 484535 (SEQ ID No 485-486), 666128 (SEQ ID No 360-361), 682276 (SEQ ID No 326), 713831 (SEQ ID No 414-415), 729542 (SEQ ID No 430-431), 838611 (SEQ ID No 336-337)

### General Methods for Quantifying the Expression Level of Disease-Specific Marker Genes

Any one of the methods known by the one skilled in the art for quantifying a nucleic acid biological marker encompassed herein may be used for performing the in vitro prediction or diagnosis method of the invention. Thus any one of the standard and non-standard (emerging) techniques well known in the art for detecting and quantifying a protein or a nucleic acid in a sample can readily be applied.

Such techniques include detection and quantification of nucleic acid biological markers with nucleic probes or primers.

The expression level of a disease-specific marker gene described herein may be quantified by any one of a wide variety of well known methods for detecting expression of a transcribed nucleic acid. Non-limiting examples of such methods include nucleic acid hybridisation methods, nucleic acid reverse transcription methods, and nucleic acid amplification methods.

In certain embodiments, the expression level of a disease-specific marker gene is assessed by preparing mRNA/cDNA (i.e. a transcribed polynucleotide) from cells originating from a thyroid tissue sample of a patient to be tested, and by hybridising the mRNA/cDNA with a reference polynucleotide which is a complement of a marker nucleic acid, or a fragment thereof, the said marker nucleic acid being comprised in the expression product of a disease-specific marker gene included in one of groups (i) to (xi) described herein. cDNA can, optionally, be amplified using any of a variety of polymerase chain reaction methods prior to hybridisation with the reference polynucleotide.

In some preferred embodiments of the in vitro prediction or diagnosis method according to the invention, step b) of expression level quantification of two or more disease-specific marker genes is performed using DNA microarrays. Illustratively, according to this preferred embodiment, a mixture of transcribed polynucleotides obtained from the thyroid tissue sample, or alternatively a mixture of the corresponding cDNAs, is contacted with a substrate having fixed thereto a plurality of polynucleotides, each of these polynucleotides consisting of a polynucleotide complementary to, or homologous with, at least a portion (e.g. at least 7, 10, 15, 20, 25, 30, 40, 50, 100, 500, or more consecutive nucleotide residues) of a disease-specific marker gene. If polynucleotides complementary to or homologous with are differentially detectable on the substrate (e.g. detectable using different chromophores or fluorophores, or fixed to different selected positions), then the levels of expression of a plurality of disease-specific marker genes can be quantified simultaneously using a single substrate (e.g. a “gene chip” microarray of polynucleotides fixed at selected positions). When a method of assessing marker expression is used which involves hybridisation of one nucleic acid with another, it is preferred that the hybridisation be performed under stringent hybridisation conditions.

In certain embodiments of the in vitro prediction or diagnosis method according to the invention, step b) comprises the steps of:
b1) providing two or more sets of nucleic acids, each nucleic acid contained in a set hybridizing specifically with a nucleic acid expression product of a disease-specific marker gene comprised in one of groups (i) to (xi);
b2) reacting the sets of nucleic acids provided at step b1) with nucleic acid expression products that are previously extracted from the thyroid tissue sample provided at step a);
b3) detecting and quantifying the nucleic acid complexes formed between (i) the sets of nucleic acids provided at step b1) and (ii) the nucleic acid expression products that are extracted from the thyroid tissue sample provided at step a);

The nucleic acids that are provided at step b1) may also be conventionally termed nucleic acid probes, each nucleic acid probe having the ability to specifically hybridize with an expression product (mRNA or cDNA) from a disease-specific marker gene selected from the group consisting of the disease-specific marker genes comprised in groups (i) to (xi) described in the present specification.

A “set” of nucleic acids that is provided at step b1) consists of one or more nucleic acids (e.g. nucleic acid probes) that all hybridize with an expression product from the same disease-specific marker gene. In the embodiments wherein a set of nucleic acids comprises two or more nucleic acids, the said nucleic acids may be identical or distinct. In the embodiments wherein a set of nucleic acids comprises two or more distinct nucleic acids, the said distinct nucleic acids preferably hybridize with distinct nucleic acid portions, most preferably non-overlapping portions, of the expression product of the same disease-specific marker gene.

As it will be described in detail below, preferred embodiments of steps b1) to b3) are performed with DNA microarrays.

Thus, in most preferred embodiments of step b) of the in vitro prediction or diagnosis method according to the invention, the expression level quantification of the disease-specific marker genes is performed by using suitable DNA microarrays. In such a marker detection/quantification format, the mRNA is immobilised on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, is both qualitative and quantitative.

Suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilised component is added to the solid phase upon which the second component is anchored. After the reaction is complete, uncomplexed components may be removed (e.g., by washing) under conditions such that any complexes formed will remain immobilised upon the solid phase. The detection of marker/probe complexes anchored to the solid phase can be accomplished in a number of methods outlined herein.

In a preferred embodiment, the probe, when it is the unanchored assay component, can be labelled for the purpose of detection and readout of the assay, either directly or indirectly, with detectable labels discussed herein and which are well-known to one skilled in the art.

In another embodiment, determination of the ability of a probe to recognise a marker can be accomplished without labelling either assay component (probe or marker) by utilising a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labelling any of the interactants (e.g., BlAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N.H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. SELDI-TOF technique may also be employed on matrix or beads coupled with active surface, or not, or antibody coated surface, or beads.

Appropriate conditions to the particular assay and components thereof will be well known to one skilled in the art.

As already mentioned above, preferred expression quantification methods use isolated RNA. For in vitro methods, any RNA isolation technique that does not select against the isolation of mRNA can be utilised for the purification of RNA from thyroid tissue sample (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

An alternative method for determining the level of mRNA marker in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

As an alternative to making determinations based on the absolute expression level of the marker, determinations may be based on the normalised expression level of the marker. Expression levels are normalised by correcting the absolute expression level of a marker by comparing its expression to the expression of a gene that is not a marker, e.g., a housekeeping gene that is constitutively expressed. Suitable genes for normalisation include housekeeping genes such as the actin gene and the ribosomal 18S gene. This normalisation allows the comparison of the expression level of one or more tissue-specific biological marker of interest in one sample.

The most preferred methods for quantifying a biological marker for the purpose of carrying out the metastasis prediction method of the invention are described hereunder.

Quantifying the Expression Level of Disease-Specific Marker Genes by cDNA Microarrays

According to this embodiment, a microarray may be constructed based on the disease-specific marker genes that are disclosed throughout the present specification. Preferably, oligonucleotide probes that specifically hybridize with the expression products (mRNA or cDNA) from each of the disease-specific marker genes tested are immobilized on a solid support, most preferably on an ordered arrangement, so as to manufacture the DNA microarray. These marker gene-specific detection probes should be designed and used in conditions such that only nucleic acids having a disease-specific marker gene sequence may hybridize and give a positive result.

Most existing microarrays, such as those provided by Affymetrix (California), may be used with the present invention.

One of skill in the art will appreciate that an enormous number of array designs are suitable. The high density array will typically include a number of probes that specifically hybridize to the sequences of interest. See WO 99/32660 for methods of producing probes for a given gene or genes. In a preferred embodiment, the array will include one or more control probes.

### Nucleic Acid Probes Immobilized on the Microarray Devices

High density array chips include <<test probes>> that specifically hybridize with mRNAs or cDNAs consisting of the products of expression of the meatstasis-specific biological markers that are described herein.

Test probes may be oligonucleotides that range from about 5 to about 500 or about 5 to about 50 nucleotides, more preferably from about 10 to about 40 nucleotides and most preferably from about 15 to about 40 nucleotides in length. In other particularly preferred embodiments, the probes are about 20 or 25 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the metastasis-specific markers whose expression they are designed to detect.

In addition to test probes that bind the target nucleic acid(s) of interest, the high density array can contain a number of control probes. The control probes fall into three categories referred to herein as normalization controls; expression level controls; and mismatch controls. Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g. fluorescence intensity) read from all other probes in the array are divided by the signal (, fluorescence intensity) from the control probes thereby normalizing the measurements. Virtually any probe may serve as a normalization control. However, it is recognized that hybridization efficiency varies with base composition and probe length. Preferred normalization probes are selected to reflect the average length of the other probes present in the array; however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes. Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including the .beta.-actin gene, the transferrin receptor gene, and the GAPDH gene. Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch). Mismatch probes thus provide a control for non-specific binding or cross hybridization to a nucleic acid in the sample other than the target to which the probe is directed. Mismatch probes also indicate whether hybridization is specific or not.

### Solid Supports for DNA Microarrays

Solid supports containing oligonucleotide probes for differentially expressed genes can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available. Any solid surface to which oligonucleotides can be bound, either directly or indirectly, either covalently or non-covalently, can be used. In some embodiments, it may be desirable to attach some oligonucleotides covalently and others non-covalently to the same solid support. A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support or the area within which the probes are attached may be on the order of a square centimeter. Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see U.S. Pat. No. 5,143,854 to Pirrung et al.; U.S. Pat. No. 5,800,992 to Fodor et al.; U.S. Pat. No. 5,837,832 to Chee et al.

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithographic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, methods which can be used to generate an array of oligonucleotides on a single substrate are described in WO 93/09668 to Fodor et al. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Oligonucleotide probe arrays for expression monitoring can be made and used according to any techniques known in the art (see for example, Lockhart et al., Nat. Biotechnol. 14, 1675-1680 (1996); McGall et al., Proc. Nat. Acad. Sci. USA 93, 13555-13460 (1996). Such probe arrays may contain at least two or more oligonucleotides that are complementary to or hybridize to two or more of the genes described herein. Such arrays may also contain oligonucleotides that are complementary to or hybridize to at least 3, 4, 5, 6, 7, 8, 9, 10, 20, 30, 50, 70, 80, 90, 100, 110, 120, 130, 140, 150, 200, 210, 220, 230, 240, 250, 260, 270, 280, 290, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000 or more of the disease-specific marker genes described therein.

### Hybridization

Nucleic acid hybridization simply involves contacting a probe and target nucleic acid under conditions where the probe and its complementary target can form stable hybrid duplexes through complementary base pairing (see WO 99/32660 to Lockhart). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6.times.SSPE-T at 37.degree. C. (0.005% Triton x-100) to ensure hybridization and then subsequent washes are performed at higher stringency (e.g., 1.times.SSPE-T at 37.degree. C.) to eliminate mismatched hybrid duplexes. Successive washes may be performed at increasingly higher stringency (e.g. down to as low as 0.25.times.SSPE-T at 37.degree. C. to 50.degree. C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level controls, normalization controls, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

### Signal Detection

The hybridized nucleic acids are typically detected by detecting one or more labels attached to the sample nucleic acids. The labels may be incorporated by any of a number of means well known to those of skill in the art (see WO 99/32660 to Lockhart). Any suitable methods can be used to detect one or more of the markers described herein. For example, gas phase ion spectrometry can be used. This technique includes, e.g., laser desorption/ionization mass spectrometry. In some embodiments, the sample can be prepared prior to gas phase ion spectrometry, e.g., pre-fractionation, two-dimensional gel chromatography, high performance liquid chromatography, etc. to assist detection of markers.

### Quantifying the Expression Level of Disease-Specific Marker Genes by Nucleic Acid Amplification

In certain embodiments, the expression level of a disease-specific marker gene, or of a set of disease-specific marker genes, may be quantified with any one of the nucleic acid amplification methods known in the art.

The polymerase chain reaction (PCR) is a highly sensitive- and powerful method for such biological markers quantification

For performing any one of the nucleic acid amplification method that is appropriate for quantifying a biological marker when performing the in vitro prediction or diagnosis method of the invention, a pair of primers that specifically hybridise with the target mRNA or with the target cDNA is required.

A pair of primers that specifically hybridise with the target nucleic acid biological marker of interest may be designed by any one of the numerous methods known in the art. Illustratively, primers that specifically hybridize with a disease-specific marker gene described herein may be easily designed by the one skilled in the art, on the basis of the nucleic acid sequence of the said disease-specific marker gene, like it is found for example in the IMAGE Consortium database, or alternatively in the HGCN database, or also after simply sequencing the DNA insert contained in the corresponding IMAGE phagemid vector.

In certain embodiments, for each of the biological markers of the invention, at least one pair of specific primers, as well as the corresponding detection nucleic acid probe, is already referenced and entirely described in the public “Quantitative PCR primer database”, notably at the following Internet address: http://lpgws.nci.nih.gov/cgi-bin/PrimerViewer.

In other embodiments, a specific pair of primers may be designed using the method disclosed in the U.S. Pat. No. 6,892,141 to Nakae et al., the entire disclosure of which is herein incorporated by reference.

Many specific adaptations of the PCR technique are known in the art for both qualitative and quantitative detection purposes. In particular, methods are known to utilise fluorescent dyes for detecting and quantifying amplified PCR products. In situ amplification and detection, also known as homogenous PCR, have also been previously described. See e.g. Higuchi et al., (Kinetics PCR Analysis: Real-time Monitoring of DNA Amplification Reactions, Bio/Technology, Vol 11, pp 1026-1030 (1993)), Ishiguro et al., (Homogeneous quantitative Assay of Hepatitis C Virus RNA by Polymerase Chain Reaction in the Presence of a Fluorescent Intercalater, Anal. Biochemistry 229, pp 20-213 (1995)), and Wittwer et al., (Continuous Fluorescence Monitoring of Rapid cycle DNA Amplification, Biotechniques, vol. 22, pp 130-138 (1997.))

A number of other methods have also been developed to quantify nucleic acids (Southern, E. M., J. Mol. Biol., 98:503-517, 1975; Sharp, P. A., et al., Methods Enzymol. 65:750-768, 1980; Thomas, P. S., Proc. Nat. Acad. Sci., 77:5201-5205, 1980). More recently, PCR and RT-PCR methods have been developed which are capable of measuring the amount of a nucleic acid in a sample. One approach, for example, measures PCR product quantity in the log phase of the reaction before the formation of reaction products plateaus (Kellogg, D. E., et al., Anal. Biochem. 189:202-208 (1990); and Pang, S., et al., Nature 343:85-89 (1990)). A gene sequence contained in all samples at relatively constant quantity is typically utilised for sample amplification efficiency normalisation. This approach, however, suffers from several drawbacks. The method requires that each sample have equal input amounts of the nucleic acid and that the amplification efficiency between samples be identical until the time of analysis. Furthermore, it is difficult using the conventional methods of PCR quantitation such as gel electrophoresis or plate capture hybridisation to determine that all samples are in fact analysed during the log phase of the reaction as required by the method.

Another method called quantitative competitive (QC)-PCR, as the name implies, relies on the inclusion of an internal control competitor in each reaction (Becker-Andre, M., Meth. Mol. Cell Biol. 2:189-201 (1991); Piatak, M. J., et al., BioTechniques 14:70-81 (1993); and Piatak, M. J., et al., Science 259:1749-1754 (1993)). The efficiency of each reaction is normalised to the internal competitor. A known amount of internal competitor is typically added to each sample. The unknown target PCR product is compared with the known competitor PCR product to obtain relative quantitation. A difficulty with this general approach lies in developing an internal control that amplifies with the same efficiency of the target molecule.

For instance, the nucleic acid amplification method that is used may consist of Real-Time quantitative PCR analysis.

Real-time or quantitative PCR (QPCR) allows quantification of starting amounts of DNA, cDNA, or RNA templates. QPCR is based on the detection of a fluorescent reporter molecule that increases as PCR product accumulates with each cycle of amplification. Fluorescent reporter molecules include dyes that bind double-stranded DNA (i.e. SYBR Green I) or sequence-specific probes (i.e. Molecular Beacons or TaqMan® Probes).

Preferred nucleic acid amplification methods are quantitative PCR amplification methods, including multiplex quantitative PCR method such as the technique disclosed in the published US patent Application no US 2005/0089862, to Therianos et al., the entire disclosure of which is herein incorporated by reference.

Illustratively, for quantifying biological markers of the invention, tumor tissue samples are snap-frozen shortly after biopsy collection. Then, total RNA from a “thyroid tissue sample” is isolated and quantified. Then, each sample of the extracted and quantified RNA is reverse-transcribed and the resulting cDNA is amplified by PCR, using a pair of specific primers for each biological marker that is quantified. Control pair of primers are simultaneously used as controls, such as pair of primers that specifically hybridise with TBP cDNA, 18S cDNA and GADPH cDNA, or any other well known “housekeeping” gene.

Illustrative embodiments of quantification of the expression level of the disease-specific marker genes described herein are disclosed in the examples herein.

### Kits for Predicting or Diagnosing the Occurrence of a Thyroid Disease

The invention also relates to a kit for the in vitro prediction or diagnosis of the occurrence of a thyroid disease in a patient (e.g. in a thyroid tissue sample previously collected from a patient to be tested). The kit comprises a plurality of reagents, each of which is capable of binding specifically with a nucleic acid that is comprised in an expression product (mRNA or cDNA) from a disease-specific marker gene selected from the disease-specific marker genes included in groups (i) to (xi) described herein.

Suitable reagents for binding with a marker nucleic acid (e.g. a mRNA, a spliced mRNA, a cDNA, or the like) include complementary nucleic acids. For example, the nucleic acid reagents may include oligonucleotides (labelled or non-labelled) fixed to a substrate, labelled oligonucleotides not bound with a substrate, pairs of PCR primers, molecular beacon probes, and the like.

Another object of the present invention consists of a kit for the in vitro prediction or diagnosis of the occurrence of a thyroid disease in a patient, which kit comprises means for quantifying the expression level of two or more disease-specific marker genes that are indicative of the risk of occurrence of, or of the occurrence of, a specific thyroid disease, wherein the said two or more disease-specific marker genes are selected from two or more of the groups of marker genes consisting of:
(i) marker genes specific for autoimmune thyroiditis (AT) that are contained as inserted DNA in the recombinant vectors selected from the group of vectors having the following IMAGE clone ID No already specified previously in the present specification;
(ii) marker genes specific for Grave's disease (GD that are contained as inserted DNA in the recombinant vectors selected from the group of vectors having the following IMAGE clone ID No already specified previously in the present specification;
(iii) marker genes specific for macrofollicular adenoma (FTA-a) that are contained as inserted DNA in the recombinant vectors selected from the group of vectors having the following IMAGE clone ID No already specified previously in the present specification;
(iv) marker genes specific for atypical follicular adenoma (FTA-aty) that are contained as inserted DNA in the recombinant vectors selected from the group of vectors having the following IMAGE clone ID No already specified previously in the present specification;
(v) marker genes specific for microfollicular adenoma (FTA-b) that are contained as inserted DNA in the recombinant vectors selected from the group of vectors having the following IMAGE clone ID No already specified previously in the present specification;
(vi) marker genes specific for follicular carcinoma (FTC) that are contained as inserted DNA in the recombinant vectors selected from the group of vectors having the following IMAGE clone ID No already specified previously in the present specification;
(vii) marker genes specific for multinodular goitres (MNG) that are contained as inserted DNA in the recombinant vectors selected from the group of vectors having the following IMAGE clone ID No already specified previously in the present specification;
(viii) marker genes specific for oncocytic adenoma (OTA) that are contained as inserted DNA in the recombinant vectors selected from the group of vectors having the following IMAGE clone ID No already specified previously in the present specification;
(ix) marker genes specific for atypical oncocytic adenoma (OTA-aty) that are contained as inserted DNA in the recombinant vectors selected from the group of vectors having the following IMAGE clone ID No already specified previously in the present specification;
(x) marker genes specific for oncocytic varcinoma (OTC) that are contained as inserted DNA in the recombinant vectors selected from the group of vectors having the following IMAGE clone ID No already specified previoulsy in the present specification; and
(xi) marker genes specific for papillary carcinoma (PTC) that are contained as inserted DNA in the recombinant vectors selected from the group of vectors having the following IMAGE clone ID No already specified previously in the present specification.

The present invention also encompasses various alternative embodiments of the said prediction or diagnosis kit, wherein the said kit comprises combination of marker quantification means, for quantifying the expression level of various combinations of the disease-specific marker genes that are described in the present specification.

Most preferably, a prediction or diagnosis kit according to the invention consists of a DNA microarray comprising probes hybridizing to the nucleic acid expression products (mRNAs or cDNAs) of the disease-specific gene markers described herein.

This invention also pertains to a collection of nucleic acids that is useful for predicting or diagnosing the occurrence of a thyroid disease in a patient, wherein the said collection of nucleic acids comprises a combination of at least two distinct nucleic acids, each distinct nucleic acid hybridizing specifically with a disease-specific marker gene described herein, belonging to a group of disease-specific marker genes selected from groups (i) to (xi).

In certain embodiments of the said collection of nucleic acids, each of the said nucleic acids consists of an IMAGE vector selected among the IMAGE vectors described herein, each IMAGE vector being used as a nucleic acid probe that specifically hybridises with an expression product of a given disease-specific gene.

In certain other embodiments of the said collection of nucleic acids, each of the said nucleic acid is selected from the group of nucleic acids consisting of SEQ ID No 3 to SEQ ID No 486 that are described elsewhere in the present specification.

Another object of the present invention consists of a kit for monitoring the effectiveness of a therapeutic treatment of a patient affected with a thyroid disease, and especially a thyroid tumor, with a pharmaceutical agent, which kit comprises means for quantifying one or more disease-specific marker genes that are indicative of the occurrence of a specific thyroid disease selected in the suitable specific group of disease-specific marker genes, among groups (i) to (xi) described herein. Illustratively, if the patient to be monitored has been diagnosed as being affected with autoimmune thyroiditis, then the monitoring kit comprises means (e.g. specific nucleic acid probes) for quantifying the expression level of two or more disease-specific marker genes belonging to group (i) of marker genes that is described herein.

The present invention also encompasses various alternative embodiments of the said monitoring kit, wherein the said monitoring kit comprises combination of marker detection and/or marker quantification means, for detecting and/or quantifying various combinations of the disease-specific marker genes described in the present specification.

In preferred embodiments, a kit according to the invention comprises (i) a combination or a set of specific nucleic acid probes or (ii) a combination or a set of nucleic acid primers, each kind of probes of primers hybridising specifically with the expression product (mRNA or cDNA) of a disease-specific marker gene selected from the disease-specific marker genes described herein.

In certain embodiments wherein nucleic acid probes are used, including when these probes are immobilised in an arrayed ordering on a solid support, the said marker gene-specific probes may consists of the whole corresponding IMAGE vectors that comprise a marker gene-specific nucleic acid inserted therein.

In other embodiments wherein nucleic acid probes are used, specifically dedicated DNA microarrays may be manufactured by immobilising on a solid support the suitable set of gene-specific probes, the said gene-specific probes being either (i) the cDNA inserts that have been previously excised from the IMAGE vectors of interest, or (ii) nucleic acid fragments thereof comprising 12 or more consecutive nucleotides thereof.

In other embodiments, the said kit comprises a combination or a set of pair of primers comprising at least two kind of pair of primers, each kind of pair of primers being selected from the group consisting of pair of primers hybridising with each of the selected disease-specific marker genes among those disclosed in the present specification.

A primer kit according to the invention may comprise 2 to 20 kinds of pair or primers, each kind of pair of primers hybridising specifically with one biological marker of the invention. For instance, a primer kit according to the invention may comprise 2, 3, 4, 5, 6, 7, 8, 9, 10, 11, 12, 13, 14, 15, 16, 17, 18, 19 or 20 kinds of pairs of primers, each kind of pair of primers hybridising specifically with a single disease-specific marker gene as disclosed herein.

Notably, at least one pair of specific primers, as well as the corresponding detection nucleic acid probe, that hybridise specifically with one disease-specific marker gene of interest, is already referenced and entirely described in the public “Quantitative PCR primer database”, notably at the following Internet address: http://lpgws.nci.nih.gov/cgi-bin/PrimerViewer.

Illustrative nucleic acids that are directly usable as primers, or alternatively that are usable for designing primers, consist of the nucleic acids of SEQ ID No 3 to SEQ ID No 486 that are disclosed herein.

### Monitoring Anti-Cancer Treatments

Monitoring the influence of agents (e.g., drug compounds) on the level of expression of one or more disease-specific marker genes identified according to the invention can be applied for monitoring the progression of the already diagnosed thyroid disease of the patient with time, which includes the development or the regression of the said thyroid disease. For example, the effectiveness of an agent to affect disease-specific marker gene expression can be monitored during treatments of subjects receiving treatments against the diagnosed specific thyroid disease, and especially treatments against the diagnosed specific thyroid tumor.

In certain embodiments, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration thyroid tissue sample from a subject prior to administration of the agent; (ii) quantifying the level of expression of one or more selected disease-specific marker genes of the invention in the pre-administration thyroid tissues sample; (iii) obtaining one or more post-administration thyroid tissue samples from the subject; (iv) quantifying the level of expression of the disease-specific marker genes in the post-administration samples; (v) comparing the level of expression of the disease-specific marker genes in the pre-administration sample with the level of expression of the disease-specific marker genes in the post-administration sample or samples; and (vi) altering the administration of the agent to the subject accordingly.

Because repeated collection of biological samples from the patient affected with a thyroid disease are needed for performing the monitoring method described above, then preferred biological samples consist of thyroid tissue samples collected by conventional needle aspiration form the subject patient.

Accordingly, the present invention also relates to a method for adapting a pharmaceutical treatment in a patient affected with a thyroid disease, wherein said method comprises the steps of:
a) performing, on at least one thyroid tissue sample collected from the said patient, the in vitro prediction or diagnosis method that is disclosed herein;
b) adapting the pharmaceutical treatment of the said patient by (i) increasing or decreasing the amounts of the pharmaceutical agent, (ii) combining the said pharmaceutical agent with one or more distinct pharmaceutical agent(s) or (iii) replacing the said pharmaceutical agent by one or more distinct pharmaceutical agent(s).

### Methods for Selecting Biological Markers Indicative of Metastasis

This invention also pertains to methods for selecting one or more disease-specific marker genes that are indicative of the occurrence of, or of the risk of occurrence of, a thyroid disease in a patient.

The said disease-specific marker gene selection method according to the invention preferably comprises the steps of:
a) providing means for quantifying one or more candidate marker genes in a thyroid tissue sample;
b) providing a plurality of collections of thyroid tissue samples originating from patients affected with a thyroid disease, wherein each of the said collections consists of a plurality of thyroid tissue samples originating from patients that are affected with a single specific thyroid disease, preferably selected from the group of thyroid diseases consisting of (i) autoimmune thyroiditis (AT), (ii) Grave's disease (GD), macrofollicular adenoma (FTA-a), (iv) atypical follicular adenoma (FTA-aty), (v) microfollicular adenoma (FTA-b), (vi) follicular carcinoma (FTC), (vii) multinodular goitres (MNG), (viii) oncocytic adenoma (OTA), (ix) atypical oncocytic adenoma (OTA-aty), (x) oncocytic carcinoma (OTC) and (xi) papillary carcinoma (PTC);
c) quantifying the expression level of each of the one or more candidate marker genes, separately in every thyroid tissue sample contained in each collection of thyroid tissue samples;
d) selecting, in the group of candidate marker genes whose expression level is quantified at step c), those marker genes that are under-expressed or over-expressed exclusively in only one collection of thyroid tissue samples that is comprised in the plurality of collections of tissue samples provided at step b), whereby a set of marker genes is selected for each collection of tissue samples, the said set of marker genes comprising disease-specific marker genes, the under-expression of which, or the over-expression of which, is indicative of the occurrence of, or of the risk of occurrence of, a thyroid disease in the said patient.

Illustrative embodiments of the selection method above are fully described in the examples herein.

For performing step a) of the selection method above, the marker quantification means encompass means for quantifying marker gene-specific nucleic acids, such as oligonucleotide primers or probes. Illustratively, DNA microarrays may be used at step a) of the selection method above.

Means for specifically quantifying any one of the known potential marker gene, e.g. any gene-specific nucleic acid, may be provided at step a) of the selection method.

Each collection of thyroid tissue samples that is provided at step b) of the selection method above comprises a number of thyroid tissue samples originating from individuals that are all affected with the same thyroid disease, the said disease being preferably selected form the group consisting of (i) autoimmune thyroiditis (AT), (ii) Grave's disease (GD), macrofollicular adenoma (FTA-a), (iv) atypical follicular adenoma (FTA-aty), (v) microfollicular adenoma (FTA-b), (vi) follicular carcinoma (FTC), (vii) multinodular goitres (MNG), (viii) oncocytic adenoma (OTA), (ix) atypical oncocytic adenoma (OTA-aty), (x) oncocytic carcinoma (OTC) and (xi) papillary carcinoma (PTC).

Preferably, each collection of thyroid tissue samples comprises samples originating from at least 5 distinct individuals affected with the same thyroid disease, and most preferably at least 20, 25 or 30 distinct individuals affected with the same thyroid disease. The statistical relevance of the disease-specific marker genes that are finally selected at the end of the selection method generally increases with the number of distinct individuals tested, and thus with the number of thyroid tissue samples comprised in each collection that is provided at step b).

At step c), quantification of the candidate marker genes on the thyroid tissue samples provided at step b), using the quantification means provided at step a), may be performed according to any one of the quantification methods that are described elsewhere in the present specification.

At step d), each candidate marker gene quantified at step c) in a first specific collection of thyroid tissue samples, (e.g. thyroid tissue samples previously collected in patients affected with autoimmune thyroiditis (AT)) is compared to the quantification results found for the same marker in all of the other collections of thyroid tissue samples, e.g. collections of thyroid tissue samples from individuals affected with GD, FTA-a, FTA-aty, FTA-b, FTC, MNG, OTA, OTA-aty, OTC and PTC, as well as a collection of thyroid tissue samples originating form individuals not affected with a thyroid disease. Then, only those candidate marker genes that are differentially expressed (i.e. (i) under-expressed, (ii) not expressed, (iii) over-expressed in the said first collection of thyroid tissue samples, as compared to the other collections of thyroid tissue samples, are positively selected as disease-specific marker genes indicative of the occurrence of a specific thyroid disease (e.g. autoimmune thyroiditis (AT)). At step d), the selection of statistically relevant disease-specific marker genes, by comparing the expression level of a candidate marker gene in one collection of thyroid tissue samples with the expression level of the said candidate marker gene in every other collection of thyroid tissue samples, is termed a “One Versus All” (“OVA”) pairwise comparison, as it is fully described in the examples herein.

The statistical relevance of each candidate marker gene tested, at step d), may be performed by calculating the p value for the said marker, for example using a univariate t-test, as disclosed in the examples herein. Generally, a marker is selected at step d) of the selection method above, when its p value is lower than 0.05.

The statistical relevance of the marker selection, at step d) of the method, may be further increased by using other statistical methods, wherein the said other statistical methods may consist of performing a multivariate permutation test, so as to provide 90% confidence that a false marker selection rate is less than 10%.

For further increasing the statistical relevance of the markers initially selected, at step d) of the selection method above, those markers that were initially selected as described above may be submitted to a further cycle of selection, for example by assaying the initially selected markers on further collections of thyroid tissue samples. This further cycle of selection may consist of, for example, performing a further expression analysis of the initially selected markers, for example by technique of quantitative RT-PCR expression analysis or by using DNA microarrays.

According to such a quantitative expression analysis, the quantification measure of expression of each initially selected marker may be normalised against a control value, e.g. the quantification measure of expression of a control gene such as TBP. The results may be expressed as N-fold difference of each marker relative to the value in normal thyroid tissues or to the value in all other thyroid tissues (normal and disease). Statistical relevance of each initially selected marker is then confirmed, for example at confidence levels of more than 95% (P of less than 0.05) using the Mann-Whitney U Test.

The present invention is further illustrated by, without in any way being limited to, the examples below.

### EXAMPLES

### A. Materials and Methods of the Examples

### A.1. Tissue Samples

Samples were obtained from 132 human thyroid tumors and 34 controls. We studied follicular adenomas exclusively: 26 macrofollicular, 17 microfollicular and 10 atypical adenomas. Atypical adenomas were defined by nuclear features and vascular or cellular modification without capsular and/or vascular invasion. We included 24 adenomas with the largest nodule originating from multinodular goiters, with macrofollicular (23 cases) or microfollicular features (1 case). We examined 30 oncocytic follicular adenomas and 5 atypical oncocytic adenomas defined using the same criteria as for non-oncocytic adenomas. Mitochondrial quantity was appreciated by immunohistochemistry, using monoclonal anti-cytochrome c oxydase antibody (Clone 113-1, Biogenex Laboratories, Inc., San Ramon, Calif., USA). We examined three types of carcinoma: follicular (3 cases), oncocytic (4 cases), and papillary carcinoma (13 cases). Control samples consisted of 24 wild type tissues, 5 samples of autoimmune thyroiditis, and 5 samples of Graves' disease. Table 1 summarizes the characteristics of the samples.

The diagnoses were made according to the WHO classification of tumors [23]. Seventy five anonymous samples were obtained from the Ambroise Paré Hospital (APHP, Boulogne S/Seine, France) and 67 anonymous samples from the University Hospital (Angers, France).

A.2. cDNA Arrays

### Extraction of Total RNA

Total RNA was extracted from tissue samples using a standard guanidium isothiocyanate protocol (TRIzol Reagent, Life Technologies, Inc., Gaithersburg, Md., USA) and quantified on a nanodrop apparatus. RNA integrity was checked using a Bio-Analyzer 2100 (Agilent Technologies, Waldbronn, Germany).

### Microarrays

Gene expression was analyzed by hybridization of nylon cDNA arrays with radioactive probes. The arrays contained spotted PCR products from 8,862 selected IMAGE clones (MRC Rosalind Franklin Centre for Genomics Research, GeneService, Cambridge, UK) human cDNA clones and control clones. Clones were selected on the basis of the following criteria: the 3′ location of the corresponding mRNA sequences, the same cloning vector (pT3T7), the same host bacteria, and approximately the same insert size. The majority of clones were selected such that they contained genes with a proven or putative implication in cancer or immune reactions. The IMAGE clones consisted of approximately 84% genes and 16% established sequence tags (ESTs). The control clones consisted of three differently sized polyadenylated sequences and pT7T3D cloning vectors (negative controls). PCR amplification and robotic spotting of PCR products on Hybond N⁺ membranes (Amersham Pharmacia Biotech, Little Chalfont, UK) were performed according to protocols described elsewhere [Bertucci F, Van Hulst S, Bernard K, Loriod B, Granjeaud S, Tagett R, Starkey M, Nguyen C, Jordan B, Birnbaum D: Expression scanning of an array of growth control genes in human tumor cell lines. Oncogene 1999, Vol. 18:3905-3912].

Hybridization of cDNA Arrays

cDNA arrays were first hybridized with a labeled oligonucleotide vector to determine the precise amount of cDNA accessible for hybridization in each spot. After stripping, each array was hybridized with a complex target prepared from 5 μg total RNA by simultaneous RT and [α-³³P]deoxy-CTP labeling as described elsewhere [Microarray protocol of the TAGC laboratory [http://taqc.univ-mrs.fr/pub/cancer/]. Each sample was hybridized on an individual array. After washing, hybridization images were obtained by scanning with an imaging plate device (Fuji BAS 5000, Raytest, Paris, France). Signal intensities were quantified using ArrayGauge software (Fujifilm Medical Systems, Stanford, Conn., USA).

### Normalization

All data were subjected to print-tip Lowess normalization [Yang Y H, Dudoit S, Luu P, Lin D M, Peng V, Ngai J, Speed T P: Normalization for cDNA microarray data: a robust composite method addressing single and multiple slide systematic variation. Nucleic Acids Res 2002, 30:e15.]. Complex target measurements were corrected for the amount of spotted DNA measured by the oligovector hybridization. Spots exhibiting weak oligovector hybridization (low amounts of spotted DNA) were considered as missing values. Genes with an expression similar to the background and genes with missing values over an entire sample class were withdrawn from the analysis. Between-array normalization was obtained by dividing each measurement by the median value of the array.

### A.3. Data Analysis and Statistical Methods

### Statistical Tests and Correction for Multiplicity

One-way analysis of variance (ANOVA) was used to detect differences between the 12 class means. Multiple testing effects were taken into account using the false discovery rate (FDR) controlling procedure [Benjamini Y, Yekutieli, D.: The control of the false discovery rate in multiple testing under dependency. ANN STAT 2001, 29:1165-1188]. Gene expression specificities were determined with t-tests (two-tailed 5% risk) by comparing mean class expressions with gene profiles.

### Clustering and Ontology Analysis

Hierarchical clustering of the genes was computed on median gene-centered and log-transformed data using average linkage and uncentered correlation distances. Expression levels of marker genes were normalized before computation. Computation and visualization were done with the Cluster and TreeView software [Eisen M B, Spellman P T, Brown P O, Botstein D: Cluster analysis and display of genome-wide expression patterns. Proc Natl Acad Sci USA 1998, 95:14863-14868.]. Gene ontology enrichments into gene clusters were computed by GoMiner software [Zeeberg B R, Feng W, Wang G, Wang M D, Fojo A T, Sunshine M, Narasimhan S, Kane D W, Reinhold W C, Lababidi S, et al: GoMiner: a resource for biological interpretation of genomic and proteomic data. Genome Biol 2003, 4:R28.].

### Similarity, Homogeneity and Separation Parameters

For the set of twelve classes Cᵢ, C={C₁, C₂, . . . , C₁₂}, we used average homogeneity and separation parameters, defined elsewhere in clustering algorithms [Sharan R, Shamir R: CLICK: a clustering algorithm with applications to gene expression analysis. Proc Int Conf Intell Syst Mol Biol 2000, 8:307-316.], to evaluate the data set quality. Each class Cᵢ was composed of Oⱼ samples and we defined its centroid Ōᵢ as its mean gene-expression signature. We defined the similarity S as the correlation coefficient of two gene-expression signatures from samples or centroids. The homogeneity of a given class Cᵢ was defined as:

$$H ( {C}_{i} ) = \frac{1}{‖ {C}_{i} ‖} {\sum }_{{O}_{j} ∈ {C}_{i}} S ( {O}_{j} , {\overline{O}}_{i} ) \qquad ( 1 )$$

The average homogeneity across all the classes was defined as:

$${H}_{ave} = \frac{1}{N} {\sum }_{{C}_{i} ∈ C} ‖ {C}_{i} ‖ \cdot H ( {C}_{i} ) \qquad ( 2 )$$

Finally, the average separation parameter was defined as:

$${S}_{ave} = \frac{{\sum }_{{C}_{i} \neq {C}_{j}} ( ‖ {C}_{i} ‖ \cdot ‖ {C}_{j} ‖ ) \cdot S ( {C}_{i} , {C}_{j} )}{{\sum }_{{C}_{i} \neq {C}_{j}} ‖ {C}_{i} ‖ \cdot ‖ {C}_{j} ‖} \qquad ( 3 )$$

Significance of similarity was assessed at a one-tailed 5% risk from 10,000 bootstrapped data sets. Pairwise similarity parameters were normalized by their expected value from bootstrapped distributions, clustered by Cluster software with the Spearman rank-correlation and the average linkage method, and visualized by TreeView Software.

### Sample Filtering

Each sample was scored by its similarity S to the class centroids. Samples were removed from the analysis if their maximal score was not coherent with histological results and class similarities. For instance, atypical oncocytic adenomas were not filtered if their maximal score related to classical oncocytic adenomas, whereas some were filtered if they related to thyroiditis.

### Automated Classifiers

We used automated classifiers to evaluate the predictive power of the selected genes. These analyses were performed using BRB ArrayTools developed by Dr. Richard Simon and Amy Peng. Algorithms were the Linear diagonal discriminant analysis (LDDA), the K-nearest neighbors for K=1 and K=3, and the nearest centroid (NC). We computed the prediction for several gene subsets selected by cutoffs to univariate tests (from 1E-4 to 1E-17). To evaluate classifier performances we used the values of population size (POP), the number of positive (POS) cases in the population, the size of the detection set (SET), and the number of true positives (TP) and true negatives (TN) in the detection set. The sensitivity (SEN) was given by SEN=TP/POS, and the positive predictive value (PPV) by PPV=TP/SET. Performance was determined by the classical accuracy coefficient (ACC) defined as:

$$ACC = \frac{( TP + TN )}{POP} \qquad ( 4 )$$

Finally, we compared the classical accuracy coefficient with the geometric mean (G) defined as:

G=\sqrt{SEN·PPV} (5)

For each subset of genes, we compared the 4 algorithms by assigning to them a score. This score is the sum of G values over the 12 tissue classes.

### Example 1

### Selection and Validation of Disease-Specific Marker Genes

### 1.1. Experimental Data

The total RNA from 132 samples of human thyroid tumors and 34 control tissues, detailed in table 1, was isolated and used to generate a radio-labeled cDNA, which was hybridized to microarrays containing 8,862 human cDNA clones. We analyzed the expression profiles of differential genes to identify the main signatures and compared the 12 types of thyroid tumor to set up a classification.

### Gene Expression Signatures of Thyroid Samples

We used one-way ANOVA statistical tests to detect variations in the gene expressions of the 12 classes of thyroid lesions defined by histo-pathological criteria. Since the classes were of different sizes, statistical bias tending to increase the number of false discoveries could be expected.

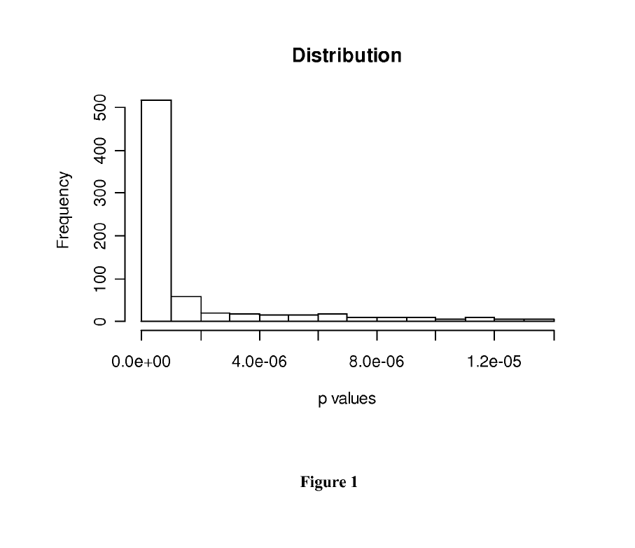

We controlled the flase discovery rate by performing multivariate permutations tests based on 10 000 random permutations. The confidence level of false discovery rate assessment was 95%. The maximum allowed number of false-positive genes was 1. From the distribution of the distribution of p values, we ranked the genes from the lowest to the highest p values (See FIG. 1). With probability of 95%, the first 714 genes contain no more than 1 false discovery.

Hierarchical clustering of the selected gene set showed the fit of gene-expression signatures on thyroid lesions, revealing strong and subtle gene expression profiles. Within-class heterogeneity was observed in all classes, but it was less in AT or FTC. Main clusters of correlated genes were identified, including some class-specific genes among which we searched for enriched gene ontology terms. Oncocytomas had consistently large signatures which mainly divided the gene set into two parts in which the genes were either over- or under-expressed. The main cluster of over-expressed genes contained the malic enzyme 3 gene (ME3), which is involved in oxidoreductase activity, the ATPase type 11B gene (ATP11B), and the neurotrophic tyrosine kinase receptor type 2 gene (NTRK2), which is related to ATP binding. The main PTC cluster of over-expressed genes contained the Cbp/p300-interacting transactivator gene (CITED1), a known papillary marker [Prasad M L, Pellegata N S, Kloos R T, Barbacioru C, Huang Y, de la Chapelle A: CITED1 protein expression suggests Papillary Thyroid Carcinoma in high throughput tissue microarray-based study. Thyroid 2004, 14:169-175]. Some of these genes also showed over-expression in FTA-aty samples. The AT signature was homogeneous and showed a large cluster of over-expressed genes, which were also over-expressed in some other samples, notably in the GD, PTC and WT classes. They included the major histocompatibility complex DP beta 1 gene (HLA-DPB1) and the chemokine ligand 19 gene (CCL19), respectively involved in immune and inflammatory responses suggestive of lymphocyte invasion. Follicular adenoma profiles (FTA-a, -b, MNG) were weakly expressed and not clearly distinguishable from each other. Despite these two drawbacks, we were able to identify an adenoma-specific cluster that included death-associated protein gene (DAP) involved in apoptosis. Over-expressed WT genes were clustered together, and included the aquaporin 11 (AQP11) gene and the solute carrier family 17 member 2 gene (SLC17A2), involved respectively in cellular volume and cellular metabolism. The other gene clusters were multi-specific.

### Comparison and Classification of Thyroid Tissues

In order to define the 12 histopathological follicular thyroid lesions, we examined each of the 66 possible pairwise comparisons of the categories. We took into account only the parameters significant at a one-tailed 5% risk based on 10,000 bootstrapped data sets. This allowed a global classification of the thyroid lesions and the visualization of all the pairwise comparisons (cells of the matrix).

The dendogram revealed three well-defined clusters of thyroid lesions grouping the benign/normal, malign and oncocytic classes by within-group similarity and inter-group dissimilarity. The benign/normal cluster grouped AT, GD, FTA-a, WT and MNG. The GD class was significantly similar to the whole cluster. Significant similarities were also found between AT and WT, and between MNG and FTA-a. In the malignant cluster, a significant similarity was observed between PTC and FTA-aty. In the oncocytic cluster, no significant similarity was found between OTA and OTC. Atypical oncocytic adenomas were similar to both OTA and OTC. The FTA-b class, which showed dissimilarities with all the other classes, was therefore considered as a singleton in this classification.

We detailed two significant similarities and dissimilarities involving either AT or OTC with FTC, OTA-aty or WT. The correlation coefficients of all sample signatures with respect to class centroids were computed and plotted on 2D graphs. Samples from similar classes were expected to be poorly separated whereas samples from dissimilar classes were expected to be sharply separated in the 2D plane. We first examined the AT and FTC classes that had been found significantly dissimilar. The graph showed the perfect separation of AT and FTC samples. We also examined the AT and WT classes that had been found significantly similar. The graph showed good correlations of all AT samples with WT (R>0.44; P-value<0.096), with 2 samples at a 5% risk. Five WT samples out of 22 had high correlations with AT (R>0.45; P-value<0.079), with 3 samples at a 5% risk. Two WT samples were clearly clustered with the AT samples. Then, we examined the dissimilar OTC and WT classes and observed the perfect separation. Finally, we examined the significantly similar OTC and OTA-aty classes. Three out of 4 OTC samples were well correlated with OTA-aty (R>0.367; P-value<0.051), with 2 samples at a 5% risk. One OTA-aty sample out of 5 correlated significantly with OTC (R=0.42; P-value<0.027).

### Filtering of Divergent Samples

Since within-class heterogeneity was observed in expression signatures, we reduced heterogeneity to obtain core class signatures by filtering the most divergent samples. The filtering consisted in removing samples which were maximally scored by the similarity parameter in an irrelevant class, with respect to histological features (Table 1), or the results of significant dissimilarities (FIG. 3A). Twenty samples were thus removed from the analysis: 12 samples excluded for a high signal of thyroiditis (7 FTA, 3 OTA and 2 WT); 2 follicular variants of PTC had been misclassified in FTA; and 4 FTA and 2 OTA samples were excluded because of high heterogeneity, presenting either oxyphilic or papillary foci in their nodules. Since 3 of the 5 GD samples were divergent, they were not excluded from the analysis.

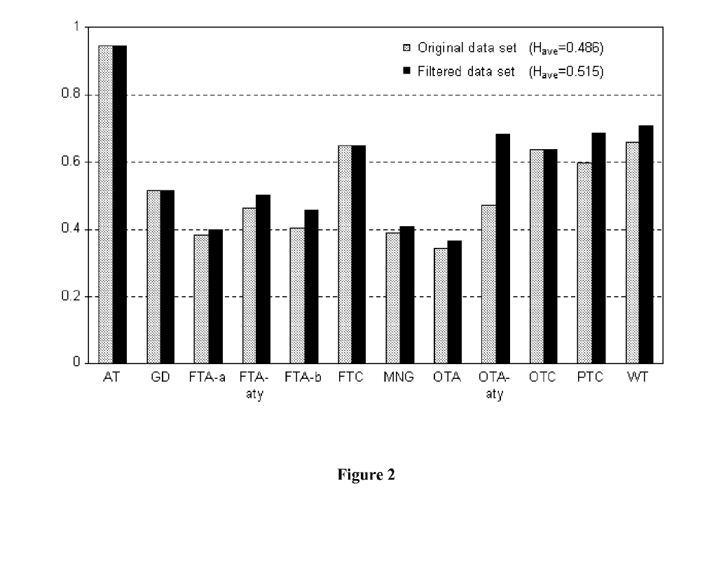

We used homogeneity parameters to compare the quality of the data sets before and after filtering of the divergent samples (FIG. 2). The average homogeneity was improved after filtering (from 0.476 to 0.515). The homogeneity of the AT, GD, FTC and OTC classes remained unchanged since no samples were excluded, while the homogeneity of all the other classes was improved. The greatest improvement (from 0.47 to 0.68) was found in the OTA-aty class, in which 2 samples out of 5 were excluded. The lowest improvements were observed in the FTA-a class (from 0.38 to 0.40), the MNG class (from 0.39 to 0.41) and the OTA class (from 0.34 to 0.37).

### Candidate Marker Genes

We used a t-statistics procedure to determine lists of candidate marker genes for each class of thyroid tumor from the filtered data set. Two lists of under- or over-expressed genes were defined at a 5% risk for each class. Gene profiles of the top 5 genes were observed in a colour-coded matrix.

The genes had heterogeneous profiles but specific expression levels in target samples. Anti-correlations of signatures were observed between classes such as FTA-a genes in OTC, FTA-aty genes in WT, FTA-b genes in AT, MNG genes in oncocytomas, OTA genes in AT or WT, PTC genes in FTA-b or WT, and WT genes in oncocytomas or PTC. Correlated signatures were also observed such as those of GD genes in WT, FTA-a genes in MNG, FTA-aty genes in PTC and vice-versa, oncocytomas with each other, and PTC genes in FTC. Oncocytic classes had similar signatures but they were distinguishable by the amplitude of expression.

### Automated Sample Prediction

We evaluated the capability of the gene-expression data to predict the class of each sample. As it was shown [Yukinawa N, Oba S, Kato K, Taniguchi K, lwao-Koizumi K, Tamaki Y, Noguchi S, Ishii S: A multi-class predictor based on a probabilistic model: application to gene expression profiling-based diagnosis of thyroid tumors. BMC Genomics 2006, 7:190.], performances of a classifier can be unstable when varying the cutoff for gene selection. We searched for the best subset of genes by observing a scoring function for 4 algorithms when varying the gene selection.

The 1-Nearest Neighbor (1-NN) classifier and the Linear Diagonal Discriminant Analysis (LDDA) showed globally the best performances. The Nearest Centroid (NC) classifier was mainly ranked the third except with poorly (>1E-7) or very stringent cutoffs (<1E-15). The 3-Nearest Neighbors classifier (3-NN) showed the worst performances. For all the classifiers, very stringent cutoffs (<1E-13) deserved the performances. For the 2 best ones, 1-NN and LDDA, the maximum score was associated to a cutoff of 1E-9 which represented 258 genes.

For this particular cutoff we ranked the classifiers relatively to each others for each tissue class (Table 2). The rank 1 was given to the best algorithm and the rank 4 to the worst (except for equality). The sum over each class indicated that LDDA had the lowest (best) ranks (Sum=21). The 1-NN classifier was very close (Sum=22). NC was the third (Sum=25) and 3-NN was the worst (Sum=43).

Finally, we detailed the performances of the LDDA and 1-NN classifiers with a gene selection at P<1 E-9. We examined two intuitive parameters as the sensitivity (SEN) and the positive predictive value (PPV). We also examined two parameters which summarized the performances as the accuracy (ACC) and the geometric mean (G). When observing SEN and PPV, performances depended dramatically of the class from quite perfect detections (AT and WT) to the worst case (OTA-aty). Though cases were very different, the ACC did not reflect it and was always higher than 0.79. For GD, the LDDA classifier had low performances (SEN=0.2, PPV=0.167, G=0.18) but a high ACC value (0.94) which was better than the good OTA case (SEN=0.74; PPV=0.87; G=0.80; ACC=0.93). For OTA-aty, no true positives were predicted by the 2 algorithms (SEN=0; PPV=0) but ACC was over 0.96 whereas G equaled 0. The ACC led to over-estimations and the geometric mean G, used in machine-learning techniques [Kubat M: Machine Learning for the Detection of Oil Spills in Satellite Radar Images. Machine Learning 1998, 30:195-215], gave results concordant with SEN and PPV (according to its definition) and proved more intuitive. So we used it to interpret the data.

The 2 algorithms showed relatively similar results. The AT and WT classes were the best cases (from G=0.89 to 0.96). OTA and PTC were well detected by 1-NN (respectively G=0.77 and G=0.73) and even more by LDDA (respectively G=0.80 and G=0.85). FTA-aty and FTA-b were also well detected and better by 1-NN (from 0.58 to 0.67). Macrofollicular adenomas (FTA-a and MNG) were detected with medium performances (from 0.38 to 0.50). GD was either badly detected by LDDA (G=0.18) or detected with medium performances by 1-NN (0.45). OTA-aty was the worst case with no detection whatever the algorithm used (G=0).

### 1.2. Detailed Discussion of the Results

Gene expression profiling is a powerful technique for finding new molecular markers for tumor growth as well as for providing insights into the molecular mechanisms involved in tumorigenesis. However, current microarray studies on thyroid tumors fail to propose truly specific markers because they are based on very incomplete sets of thyroid tumor classes. We have conducted the first microarray study including more than 90% of the types of thyroid tumor known.

Extracting Information from Data Concerning Heterogeneous and Rare Thyroid Tumors

We measured 8,862 gene expression levels for 166 samples representing 12 different classes of thyroid lesions. However, thyroid tumors are heterogeneous and subject to diagnostic misclassifications [Hirokawa M, Carney J A, Goellner J R, DeLellis R A, Heffess C S, Katoh R, Tsujimoto M, Kakudo K: Observer variation of encapsulated follicular lesions of the thyroid gland. Am J Surg Pathol 2002, 26:1508-1514; Franc B, de la Salmoniere P, Lange F, Hoang C, Louvel A, de Roquancourt A, Vilde F, Hejblum G, Chevret S, Chastang C: Interobserver and intraobserver reproducibility in the histopathology of follicular thyroid carcinoma. Hum Pathol 2003, 34:1092-1100.; Lloyd R V, Erickson L A, Casey M B, Lam K Y, Lohse C M, Asa S L, Chan J K, DeLellis R A, Harach H R, Kakudo K, et al: Observer variation in the diagnosis of follicular variant of papillary thyroid carcinoma. Am J Surg Pathol 2004, 28:1336-1340]. The classical methods of analysis are affected by weak intra-class homogeneity and by unbalanced classes since very few samples were available for the rarer pathologies. Because of the presence of rare samples, we used a stringent gene selection method to limit false discoveries due to statistical bias caused by unbalanced class sizes. This method allows the detection of bias and limits the effect by optimizing the power of the analysis.

The hierarchical clustering of the data can be a complement to supervised methods, it shows the strongest molecular signals but it fails to reveal the specificity of some of the classes of tumor, such as GD, FTA-a, FTA-b and MNG. However, useful information can be extracted from these classes with other methods such as that of global classification and the definition of several candidate markers. As the hierarchical clustering algorithm assumes that genes behave similarly in all the samples and that they are each associated with a single biological function or process [Tanay A, Sharan R, Shamir R: Discovering statistically significant biclusters in gene expression data. Bioinformatics 2002, 18 Suppl 1:S136-144], it shows limitations when many samples are involved.

Within-class gene expression signatures of thyroid tumors are heterogeneous. Filtering divergent tissues is useful for the definition of the core signatures of tissues but it prevents the analysis of heterogeneity that might otherwise bring subclasses to light. However, it leads to a better choice of class-specific candidate marker genes by improving within-class and overall homogeneity (FIG. 2). Large data sets containing both abundant and rare samples can pose a problem for predictive algorithms because of the presence of unbalanced positive and negative samples. In such cases, the interpretation of the classical accuracy coefficient (ACC) is far from intuitive and leads to over-estimation of performance. For instance, if 13 our 166 samples had actually been PTCs, and 10 samples were predicted to be PTCs whereas none were truly PTC, then the accuracy would still work out at as high as 86%. In contrast, the use of the geometric mean G is unbiased and quite intuitive [Kubat M: Machine Learning for the Detection of Oil Spills in Satellite Radar Images. Machine Learning 1998, 30:195-215.]. Thus, in the hypothetical case discussed, G would be equal to zero.

### The Classification Based on Gene Expression is Complementary to the WHO Classification

The classification based on gene expression divides the thyroid tumors into three distinct groups (benign/normal, malignant and oncocytic) and separates microfollicular adenomas (FTA-b). Pairwise distinctions of benign/normal and malign tumors are consistent with published reports, as in the case of PTC and FTA [Finley D J, Arora N, Zhu B, Gallagher L, Fahey T J, 3rd: Molecular profiling distinguishes papillary carcinoma from benign thyroid nodules. J Clin Endocrinol Metab 2004, 89:3214-3223].

Moreover, thanks to the large and almost complete set of thyroid tumors included in our study, the results may be generalized to the whole range of thyroid tumors, from the benign/normal to the malignant and oncocytic tumors. Oncocytomas are the most divergent tumors compared to normal tissue, particularly with respect to the large number of genes involved in mitochondrial activity. Moreover, since they appear unrelated to PTCs or FTCs in the classification of thyroid tumors based on gene expression (FIG. 3A), they should be analyzed independently [Giordano T J, Au A Y, Kuick R, Thomas D G, Rhodes D R, Wilhelm K G, Jr., Vinco M, Misek D E, Sanders D, Zhu Z, et al: Delineation, functional validation, and bioinformatic evaluation of gene expression in thyroid follicular carcinomas with the PAX8-PPARG translocation. Clin Cancer Res 2006, 12:1983-1993] and not be considered as PTC- or FTC-related tumors [Barden C B, Shister K W, Zhu B, Guiter G, Greenblatt D Y, Zeiger M A, Fahey T J, 3rd: Classification of follicular thyroid tumors by molecular signature: results of gene profiling. Clin Cancer Res 2003, 9:1792-1800.] in gene expression studies.

We observed that microfollicular adenoma presents a signature different from macrofollicular adenoma and follicular carcinoma. We may consider this subtype as a distinct entity because of the mechanism involved in its pathogenesis, although we do not know the impact of this specific profile on the tumoral outcome. Besides, variations in the gene expression signatures of macrofollicular adenomas (FTA-a and MNG) is subtle and lead automated classifiers to have limited performances on these 2 cases. They should be carefully considered in global expression studies. We think that it is worth investigating differences in macro- and microfollicular adenomas to understand the microfollicular machinery.

Non-oncocytic malignant and atypical tissues are clustered together in the classification. A significant similarity exists between the PTC and FTA-aty classes. No particular similarity or dissimilarity was found for the FTC class. The lack of significant results may be explained by the small size of the FTC sample; larger samples should produce more conclusive results. The close relationship between the PTC and FTA-aty classes added argument to the proposal terminology of a “differentiated tumor of uncertain malignant potential”. This designation permitted to clearly distinguish the atypical lesions for which the pending diagnosis is PTC from the others which are either non malignant or candidate to a FTC diagnosis and referred as “follicular tumor of uncertain malignant potential”.

### Markers for the Classification of Main Thyroid Pathologies

The identification of pathologic tissue by morphological features is not always precise and exclusive. In thyroid lesions, it has been shown that inter- and intra-observer variations are significant [Aldred M A, Huang Y, Liyanarachchi S, Pellegata N S, Gimm O, Jhiang S, Davuluri R V, de la Chapelle A, Eng C: Papillary and follicular thyroid carcinomas show distinctly different microarray expression profiles and can be distinguished by a minimum of five genes. J Clin Oncol 2004, 22:3531-3539; Finley D J, Lubitz C C, Wei C, Zhu B, Fahey T J, 3rd: Advancing the molecular diagnosis of thyroid nodules: defining benign lesions by molecular profiling. Thyroid 2005, 15:562-568].

Mutations and rearrangements can define a class and its evolution more precisely than the morphology, as in the case of papillary and follicular thyroid carcinomas [Giordano T J, Au A Y, Kuick R, Thomas D G, Rhodes D R, Wilhelm K G, Jr., Vinco M, Misek D E, Sanders D, Zhu Z, et al: Delineation, functional validation, and bioinformatic evaluation of gene expression in thyroid follicular carcinomas with the PAX8-PPARG translocation. Clin Cancer Res 2006, 12:1983-1993; Giordano T J, Kuick R, Thomas D G, Misek D E, Vinco M, Sanders D, Zhu Z, Ciampi R, Roh M, Shedden K, et al: Molecular classification of papillary thyroid carcinoma: distinct BRAF, RAS, and RET/PTC mutation-specific gene expression profiles discovered by DNA microarray analysis. Oncogene 2005, 24:6646-6656].

However, some histotypes like oncocytic carcinomas escape to this molecular classification [Nikiforova M N, Lynch R A, Biddinger P W, Alexander E K, Dorn G W, 2nd, Tallini G, Kroll T G, Nikiforov Y E: RAS point mutations and PAX8-PPAR gamma rearrangement in thyroid tumors: evidence for distinct molecular pathways in thyroid follicular carcinoma. J Clin Endocrinol Metab 2003, 88:2318-2326]. Several groups have evaluated the usefulness of biomarkers combination in the diagnosis of suspicious thyroid lesions [Weber, 2004; Ito, 2005; Prasad, 2005]. But they failed to show use in clinical practice, as an adjunctive test for exploring such nodules. However, development of new antibodies against selected markers has proved to accurate the diagnosis of suspicious lesions [Cerutti J M, Latini F R, Nakabashi C, Delcelo R, Andrade V P, Amadei M J, Maciel R M, Hojaij F C, Hollis D, Shoemaker J, Riggins G J: Diagnosis of suspicious thyroid nodules using four protein biomarkers. Clin Cancer Res 2006, 12:3311-3318.]. These antibodies were done after selection by gene expression profiling. That proved the invaluable assistance of this technology in choosing the most appropriate markers to distinguish between the various thyroid lesions. But this study also shows that several new custom antibodies might be produced and tested before using in clinical practice. Currently, we show that a classification of thyroid lesions based on gene expression patterns is a useful complement to the pathologic classification. We postulate that dedicated microarrays containing a subset of the differential and the predictive genes may be used to reclassify some lesions. All of the 12 classes show high predictive accuracy using our method. However, one class, the OTA-Aty class, presents a low G value Our study offers a sensitive method that may be used on fine needle aspiration for classifying thyroid lesions. Defining all of these subclasses is a close necessary for classifying follicular lesions situated in the grey zone area.

TABLE 1
Thyroid tissue samples included in the analysis

| | | | |
|---|---|---|---|
| Thyroid tissue | N | Histology | Follicle architecture |

Controls

| | | | |
|---|---|---|---|
| Wild type (WT) | 24 | Normal | Normal |
| Graves's disease (GD) | 5 | Diffuse goiter | Normal |
| Autoimmune thyroiditis (AT) | 5 | Diffuse lymphocyte thyroiditis | Normal |

Benign tumors

| | | | |
|---|---|---|---|
| Oncocytic adenoma (OTA) | 30 | Over 95% oncocytic cells and | Micro (17) or macro (13) |
| | | mitochondrial antibody ++ | |

Follicular adenomas (FTA):complete fibrous capsule

| | | | |
|---|---|---|---|
| Macrofollicular (FTA-a) | 26 | Unique nodule | Macro* |
| Microfollicular (FTA-b) | 17 | Unique nodule | Micro** |
| Multinodular goiter (MNG) | 24 | largest nodule of a multinodular goiter | Micro (1) or macro (23) |

Atypical adenomas:complete fibrous capsule and moderate vascular invasion

| | | | |
|---|---|---|---|
| Follicular (FTA-aty) | 10 | Unique nodule and atypical nucleus | Micro (1) or Macro(9) |
| Oncocytic (OTA-aty) | 5 | Unique nodule and atypical nucleus | Micro (2) or Macro (3) |

Differentiated malignant tumors:capsular and vascular invasion

| | | | |
|---|---|---|---|
| Follicular carcinoma (FTC) | 3 | Unique | Micro |
| Oncocytic carcinoma (OTC) | 4 | Over 95% oncocytic cells and | Trabecular |
| | | mitochondrial antibody ++ | |
| Papillary carcinoma (PTC) | 13 | Typical papillary nuclear features | Papillary architecture (5) |
| | | | or follicular variant (8) |

*Micro = microfollicular;
**Macro = macrofollicular

TABLE 2
Relative performance ranks of the classifiers based on
the geometric means G

| Class | LDDA* | 1-NN** | 3-NN*** | NC**** |
|---|---|---|---|---|
| AT | 1 | 2 | 4 | 3 |
| FTA-a | 2 | 1 | 4 | 3 |
| FTA-aty | 3 | 2 | 4 | 1 |
| FTA-b | 3 | 2 | 4 | 1 |
| FTC | 2 | 1 | 3 | 3 |
| GD | 2 | 1 | 3 | 3 |
| MNG | 1 | 3 | 4 | 1 |
| OTA | 1 | 2 | 4 | 3 |
| OTA-aty | 1 | 1 | 1 | 1 |
| OTC | 1 | 3 | 4 | 2 |
| PTC | 2 | 3 | 4 | 1 |
| WT | 2 | 1 | 4 | 3 |
| Sum | 21 | 22 | 43 | 25 |

*LLDA = Linear Diagonal Discriminant Analysis;
**1-NN = 1-Nearest Neighbor;
***3-NN = 3-Nearest Test Neighbor;
****NC = Nearest Centroid

TABLE 3

| | AT | Under | |
|---|---|---|---|
| name | cloid | p-value | fold |
| IQCA | 669510 | 1.04E−34 | 0.44 |
| KRT18 | 725096 | 1.15E−33 | 0.36 |
| MATN2 | 366100 | 1.08E−28 | 0.34 |
| KRTHA4 | 325155 | 1.50E−23 | 0.21 |
| FCGBP | 154172 | 1.42E−21 | 0.08 |
| SLC4A4 | 787938 | 8.75E−21 | 0.31 |
| IMAGE:744055 | 744055 | 7.13E−20 | 0.22 |
| Transcribed | | | |
| locus | | | |
| KRT7 | 592276 | 8.12E−17 | 0.27 |
| C10orf116 | 740941 | 9.03E−16 | 0.40 |
| GSTM1 | 73778 | 1.29E−15 | 0.39 |
| LOC400451 | 667174 | 1.38E−14 | 0.27 |
| CRABP1 | 739193 | 2.75E−14 | 0.12 |
| RAD51L1 | 295412 | 5.98E−14 | 0.28 |
| HSPB1 | 23827 | 1.66E−13 | 0.53 |
| IMAGE:136686 | 136686 | 1.14E−12 | 0.40 |
| IMAGE:744385 | 744385 | 1.43E−12 | 0.50 |
| Similar to | | | |
| heat shock | | | |
| 10 kDa | | | |
| protein 1 | | | |
| (chaperonin | | | |
| 10); heat | | | |
| shock 10 kD | | | |
| protein 1 | | | |
| (chaperonin | | | |
| 10) | | | |
| RODH | 471641 | 2.05E−12 | 0.18 |
| CHRAC1 | 724276 | 2.06E−12 | 0.43 |
| MATN2 | 28584 | 6.45E−12 | 0.22 |
| CTNNA1 | 268972 | 6.80E−12 | 0.63 |
| MGC99813 | 739097 | 6.83E−12 | 0.19 |
| FLJ14351 | 744943 | 8.09E−12 | 0.17 |
| PLXNC1 | 724609 | 8.10E−12 | 0.44 |
| ICOSLG | 2074228 | 4.39E−11 | 0.40 |
| IMAGE:365913 | 365913 | 4.41E−11 | 0.48 |
| Transcribed | | | |
| locus | | | |
| NFKB1 | 687782 | 6.62E−11 | 0.41 |
| IMAGE:23872 | 23872 | 9.23E−11 | 0.62 |
| AF034176 | | | |
| Human | | | |
| mRNA | | | |
| (Tripodis and | | | |
| Ragoussis) | | | |
| Homo | | | |
| sapiens | | | |
| cDNA clone | | | |
| ntcon5 contig | | | |
| FLJ23577 | 668452 | 1.01E−10 | 0.38 |
| PON3 | 681959 | 1.62E−10 | 0.39 |
| ANKRD28 | 687379 | 5.78E−10 | 0.41 |
| FLJ12895 | 23271 | 1.31E−09 | 0.53 |
| C6orf118 | 731745 | 1.63E−09 | 0.63 |
| IMAGE:738332 | 738332 | 1.88E−09 | 0.26 |
| Transcribed | | | |
| locus | | | |
| LOC440934 | 726760 | 2.25E−09 | 0.47 |
| IMAGE:666911 | 666911 | 4.05E−09 | 0.37 |
| Transcribed | | | |
| locus | | | |
| HSPB1 | 724150 | 4.15E−09 | 0.50 |
| IGF1R | 682555 | 4.46E−09 | 0.60 |
| LRPAP1 | 587186 | 8.31E−09 | 0.28 |
| GNB4 | 813260 | 9.71E−09 | 0.30 |
| NY-REN-41 | 136646 | 1.10E−08 | 0.52 |
| CAV1 | 309645 | 1.88E−08 | 0.39 |
| E2F5 | 809828 | 2.42E−08 | 0.32 |
| LRRIQ2 | 487152 | 2.49E−08 | 0.67 |
| GRB10 | 564994 | 2.81E−08 | 0.49 |
| ITPR1 | 667348 | 3.94E−08 | 0.69 |
| TCEA2 | 730149 | 5.89E−08 | 0.51 |
| SLC36A3 | 731115 | 6.86E−08 | 0.69 |
| IMAGE:738945 | 738945 | 7.57E−08 | 0.63 |
| PIP | 985457 | 8.60E−08 | 0.52 |
| TPCN1 | 179288 | 1.02E−07 | 0.51 |
| SLC22A18 | 742862 | 1.08E−07 | 0.62 |
| MT1F | 78353 | 1.17E−07 | 0.17 |
| VEGFC | 503189 | 1.53E−07 | 0.48 |
| ID1 | 1087348 | 2.20E−07 | 0.23 |
| GNAI1 | 753215 | 2.34E−07 | 0.45 |
| MRPS11 | 471574 | 2.79E−07 | 0.54 |
| KLHL9 | 471696 | 2.92E−07 | 0.31 |
| APLP2 | 549054 | 3.20E−07 | 0.30 |
| LMLN | 744657 | 3.23E−07 | 0.52 |
| IMAGE:731357 | 731357 | 3.83E−07 | 0.65 |
| Transcribed | | | |
| locus | | | |
| MGC17299 | 713422 | 4.48E−07 | 0.51 |
| FARP1 | 486708 | 4.61E−07 | 0.44 |
| IMAGE:666794 | 666794 | 4.76E−07 | 0.68 |
| Hypothetical | | | |
| LOC388170 | | | |
| FLJ10748 | 726830 | 5.32E−07 | 0.50 |
| DKFZp761P0423 | 730829 | 5.50E−07 | 0.50 |
| CLDN7 | 300268 | 6.15E−07 | 0.39 |
| PVRL2 | 725364 | 7.25E−07 | 0.60 |
| PDCD4 | 665376 | 7.38E−07 | 0.45 |
| WNT4 | 375746 | 7.99E−07 | 0.67 |
| CCND1 | 324079 | 8.65E−07 | 0.40 |
| GLT8D2 | 365271 | 9.21E−07 | 0.58 |
| EPHB6 | 172982 | 9.40E−07 | 0.48 |
| PPARD | 724562 | 9.65E−07 | 0.55 |
| GJA1 | 839101 | 9.68E−07 | 0.42 |
| CITED1 | 265558 | 1.18E−06 | 0.27 |
| LOC51760 | 52226 | 1.31E−06 | 0.56 |
| GSTM2 | 713922 | 1.40E−06 | 0.50 |
| CCL28 | 136919 | 1.53E−06 | 0.58 |
| HOXD11 | 682119 | 1.61E−06 | 0.40 |
| ANKRD20B | 745106 | 1.61E−06 | 0.46 |
| IMAGE:1877668 | 1877668 | 1.95E−06 | 0.44 |
| BMP7 | 1594414 | 1.98E−06 | 0.32 |
| RABL5 | 471829 | 2.09E−06 | 0.49 |
| FGFRL1 | 739956 | 2.18E−06 | 0.33 |
| DJ159A19.3 | 23945 | 2.27E−06 | 0.51 |
| CACNB2 | 173841 | 2.39E−06 | 0.66 |
| SASH1 | 31120 | 2.49E−06 | 0.40 |
| BCL2 | 342181 | 2.50E−06 | 0.52 |
| PEMT | 742580 | 2.68E−06 | 0.46 |
| SEMA7A | 135941 | 2.91E−06 | 0.20 |
| SLA | 32339 | 2.98E−06 | 0.36 |
| TYMS | 687912 | 3.29E−06 | 0.52 |
| PDE8A | 666154 | 3.34E−06 | 0.55 |
| IMP-2 | 743774 | 4.07E−06 | 0.40 |
| DJ462O23.2 | 738970 | 4.08E−06 | 0.57 |
| RPS6KA2 | 22711 | 4.25E−06 | 0.51 |
| YES1 | 501971 | 5.09E−06 | 0.58 |
| FLJ12998 | 364846 | 5.14E−06 | 0.56 |
| FLJ20160 | 175864 | 5.37E−06 | 0.55 |
| ARHGAP21 | 666792 | 5.79E−06 | 0.78 |
| IMAGE:724416 | 724416 | 6.99E−06 | 0.32 |
| Transcribed | | | |
| locus | | | |
| OTUB1 | 174683 | 7.07E−06 | 0.63 |
| DCI | 667892 | 7.80E−06 | 0.36 |
| PLA2G6 | 744087 | 8.56E−06 | 0.54 |
| CCNE1 | 68950 | 9.61E−06 | 0.45 |
| ZBTB16 | 2467442 | 9.79E−06 | 0.35 |
| NR2F6 | 136807 | 1.10E−05 | 0.49 |
| IMAGE:744439 | 744439 | 1.31E−05 | 0.41 |
| Transcribed | | | |
| locus | | | |
| IMAGE:1716286 | 1716286 | 1.52E−05 | 0.33 |
| LAD1 | 121551 | 1.69E−05 | 0.70 |
| SH3RF2 | 744797 | 1.97E−05 | 0.45 |
| IMAGE:742061 | 742061 | 2.01E−05 | 0.72 |
| LATS2 | 666778 | 2.21E−05 | 0.54 |
| BCL2 | 342181 | 2.28E−05 | 0.55 |
| DUSP24 | 740158 | 2.57E−05 | 0.40 |
| DCXR | 724596 | 2.78E−05 | 0.58 |
| CKLFSF4 | 143759 | 2.91E−05 | 0.41 |
| IMAGE:666315 | 666315 | 2.94E−05 | 0.60 |
| Homo | | | |
| sapiens, | | | |
| clone | | | |
| IMAGE:5312086, | | | |
| mRNA | | | |
| SNCB | 50202 | 3.77E−05 | 0.50 |
| TP53I11 | 667514 | 3.92E−05 | 0.59 |
| GFM2 | 364959 | 4.24E−05 | 0.44 |
| NR6A1 | 258666 | 4.39E−05 | 0.52 |
| GATA3 | 148796 | 4.73E−05 | 0.37 |
| PRUNE | 364324 | 4.73E−05 | 0.61 |
| NR2F2 | 72744 | 5.83E−05 | 0.38 |
| SGSH | 669263 | 6.29E−05 | 0.61 |
| HAS3 | 667533 | 6.62E−05 | 0.35 |
| FLJ32731 | 365177 | 6.76E−05 | 0.63 |
| LRP2 | 2055272 | 6.77E−05 | 0.39 |
| IMAGE:731298 | 731298 | 7.03E−05 | 0.62 |
| CDNA | | | |
| FLJ42482 fis, | | | |
| clone | | | |
| BRACE2032134 | | | |
| COX6A1 | 512910 | 7.09E−05 | 0.60 |
| CTTN | 470408 | 7.09E−05 | 0.52 |
| POLI | 137720 | 7.40E−05 | 0.38 |
| EPHA4 | 2237263 | 7.94E−05 | 0.53 |
| BAD | 712295 | 8.56E−05 | 0.65 |
| ABCA8 | 284828 | 9.00E−05 | 0.64 |
| MT1F | 81911 | 9.32E−05 | 0.22 |
| ARMCX3 | 251452 | 9.47E−05 | 0.67 |
| ZNF550 | 135705 | 9.85E−05 | 0.60 |
| CES2 | 153667 | 1.14E−04 | 0.67 |
| USP13 | 666007 | 1.14E−04 | 0.54 |
| IMAGE:2266583 | 2266583 | 1.18E−04 | 0.42 |
| SYCP2 | 136863 | 1.26E−04 | 0.53 |
| CRELD1 | 724119 | 1.31E−04 | 0.67 |
| IMAGE:136223 | 136223 | 1.45E−04 | 0.71 |
| CD9 | 727251 | 1.46E−04 | 0.47 |
| CHRDL2 | 485872 | 1.46E−04 | 0.73 |
| ANXA11 | 137238 | 1.47E−04 | 0.73 |
| MESDC2 | 713653 | 1.48E−04 | 0.60 |
| DHRS6 | 364412 | 1.48E−04 | 0.43 |
| LYPLA3 | 471702 | 1.54E−04 | 0.62 |
| IMAGE:668684 | 668684 | 1.61E−04 | 0.45 |
| Transcribed | | | |
| locus, weakly | | | |
| similar to | | | |
| XP_209041.2 | | | |
| PREDICTED: | | | |
| similar to | | | |
| KIAA1503 | | | |
| protein | | | |
| [Homo | | | |
| sapiens] | | | |
| SYN3 | 727056 | 1.63E−04 | 0.54 |
| BDKRB2 | 665674 | 1.73E−04 | 0.38 |
| TMED4 | 251250 | 1.75E−04 | 0.60 |
| APIN | 364706 | 1.77E−04 | 0.49 |
| IMMP2L | 136260 | 1.84E−04 | 0.57 |
| VPS13D | 136560 | 1.93E−04 | 0.67 |
| KIAA1196 | 738938 | 1.96E−04 | 0.59 |
| IMAGE:382423 | 382423 | 2.13E−04 | 0.61 |
| Transcribed | | | |
| locus | | | |
| RTN3 | 430263 | 2.56E−04 | 0.63 |
| BRAF | 2139164 | 2.57E−04 | 0.50 |
| ALDH1A1 | 309697 | 2.57E−04 | 0.48 |
| DDX1 | 773192 | 2.59E−04 | 0.58 |
| SSH3 | 726272 | 3.01E−04 | 0.59 |
| LOC126669 | 682088 | 3.08E−04 | 0.53 |
| PTPN4 | 666367 | 3.12E−04 | 0.58 |
| PTDSS2 | 21716 | 3.15E−04 | 0.60 |
| PDLIM1 | 135689 | 3.19E−04 | 0.51 |
| PSMD11 | 383945 | 3.44E−04 | 0.76 |
| IMAGE:727289 | 727289 | 3.46E−04 | 0.68 |
| Transcribed | | | |
| locus | | | |
| PML | 724554 | 3.85E−04 | 0.60 |
| ZNF319 | 364729 | 4.16E−04 | 0.34 |
| IMAGE:665649 | 665649 | 4.42E−04 | 0.64 |
| Transcribed | | | |
| locus | | | |
| RGS20 | 668152 | 4.63E−04 | 0.57 |
| GOLPH2 | 811582 | 4.75E−04 | 0.44 |
| LOC80298 | 666551 | 4.76E−04 | 0.54 |
| IMAGE:472111 | 472111 | 4.88E−04 | 0.43 |
| Transcribed | | | |
| locus | | | |
| CTTN | 489945 | 5.35E−04 | 0.62 |
| RASAL2 | 486304 | 5.40E−04 | 0.67 |
| CTBP2 | 416744 | 5.45E−04 | 0.67 |
| CLECSF12 | 258865 | 5.47E−04 | 0.39 |
| PSCD3 | 744050 | 5.48E−04 | 0.79 |
| KCNK4 | 743016 | 5.49E−04 | 0.65 |
| BMP5 | 1846326 | 5.51E−04 | 0.54 |
| COX8A | 509606 | 5.64E−04 | 0.57 |
| CENTD1 | 666110 | 5.81E−04 | 0.75 |
| ESRRBL1 | 51232 | 6.12E−04 | 0.53 |
| S100A2 | 3659591 | 6.31E−04 | 0.60 |
| FSHPRH1 | 667355 | 6.32E−04 | 0.59 |
| PP1201 | 364974 | 6.47E−04 | 0.58 |
| PDGFD | 666860 | 6.59E−04 | 0.74 |
| CTXN1 | 179266 | 6.77E−04 | 0.63 |
| RYK | 727092 | 7.01E−04 | 0.78 |
| RGS6 | 24176 | 7.34E−04 | 0.55 |
| NT5C2 | 725076 | 7.70E−04 | 0.73 |
| PEF | 137353 | 7.85E−04 | 0.58 |
| GCAT | 307094 | 7.89E−04 | 0.81 |
| IMAGE:23765 | 23765 | 8.14E−04 | 0.60 |
| FBXO21 | 666168 | 8.25E−04 | 0.49 |
| LRP5 | 725836 | 8.38E−04 | 0.60 |
| CREBBP | 172996 | 8.59E−04 | 0.73 |
| LOC92154 | 364926 | 8.87E−04 | 0.69 |
| ALDH3A1 | 525221 | 9.17E−04 | 0.67 |
| NIFIE14 | 472160 | 9.48E−04 | 0.51 |
| SIAT8F | 667110 | 9.49E−04 | 0.61 |
| INHBB | 730012 | 9.53E−04 | 0.52 |
| EGFR | 135980 | 9.54E−04 | 0.55 |
| IMAGE:136014 | 136014 | 9.57E−04 | 0.55 |
| SGCG | 2046361 | 1.01E−03 | 0.76 |
| CDH16 | 726763 | 1.04E−03 | 0.47 |
| IMAGE:731726 | 731726 | 1.10E−03 | 0.66 |
| Transcribed | | | |
| locus | | | |
| AXIN2 | 135887 | 1.13E−03 | 0.60 |
| MDC1 | 365288 | 1.15E−03 | 0.78 |
| TU3A | 44881 | 1.16E−03 | 0.62 |
| SLC12A8 | 724508 | 1.16E−03 | 0.59 |
| ETF1 | 146976 | 1.20E−03 | 0.69 |
| IMAGE:744616 | 744616 | 1.24E−03 | 0.58 |
| DNA | | | |
| damage | | | |
| repair and | | | |
| recombination | | | |
| protein | | | |
| RAD52 | | | |
| pseudogene | | | |
| RNF123 | 366159 | 1.28E−03 | 0.58 |
| DC12 | 724895 | 1.38E−03 | 0.80 |
| PTK2 | 724892 | 1.43E−03 | 0.59 |
| EVC2 | 668912 | 1.45E−03 | 0.84 |
| ARL6IP2 | 743987 | 1.49E−03 | 0.57 |
| IMAGE:23817 | 23817 | 1.50E−03 | 0.55 |
| CDNA | | | |
| FLJ12202 fis, | | | |
| clone | | | |
| MAMMA1000908 | | | |
| ARID1B | 665538 | 1.55E−03 | 0.72 |
| NEXN | 687625 | 1.60E−03 | 0.72 |
| HNRPF | 136606 | 1.61E−03 | 0.54 |
| MX1 | 713879 | 1.65E−03 | 0.68 |
| IMAGE:26164 | 26164 | 1.67E−03 | 0.68 |
| PRKG1 | 667587 | 1.70E−03 | 0.66 |
| DKFZp547K054 | 731410 | 1.71E−03 | 0.64 |
| IMAGE:743422 | 743422 | 1.74E−03 | 0.65 |
| FBXW8 | 687532 | 1.83E−03 | 0.62 |
| CLDN7 | 726335 | 1.85E−03 | 0.50 |
| DEPDC6 | 669318 | 1.85E−03 | 0.49 |
| SMAD4 | 214611 | 1.90E−03 | 0.55 |
| ZNF651 | 669181 | 1.90E−03 | 0.57 |
| TM4SF8 | 713647 | 1.91E−03 | 0.58 |
| SYNPO | 178792 | 1.92E−03 | 0.68 |
| ABCC8 | 1417901 | 2.05E−03 | 0.61 |
| LOC285500 | 135766 | 2.15E−03 | 0.63 |
| NDUFA7 | 364469 | 2.23E−03 | 0.60 |
| TSHR | 565317 | 2.25E−03 | 0.36 |
| ANP32E | 382738 | 2.31E−03 | 0.61 |
| IMAGE:669188 | 669188 | 2.37E−03 | 0.51 |
| PGR1 | 666361 | 2.37E−03 | 0.77 |
| FLJ13197 | 667117 | 2.38E−03 | 0.58 |
| RHOD | 591907 | 2.39E−03 | 0.56 |
| BTBD14B | 173288 | 2.48E−03 | 0.61 |
| IMAGE:257555 | 257555 | 2.54E−03 | 0.63 |
| CDH1 | 214008 | 2.70E−03 | 0.34 |
| PH-4 | 730938 | 2.75E−03 | 0.58 |
| LPL | 868169 | 2.78E−03 | 0.58 |
| LOC340073 | 731404 | 2.79E−03 | 0.55 |
| C9orf119 | 667112 | 2.84E−03 | 0.57 |
| TERF1 | 135773 | 2.86E−03 | 0.67 |
| LOC388886 | 34007 | 3.02E−03 | 0.53 |
| JAG2 | 1521706 | 3.08E−03 | 0.72 |
| LPXN | 687679 | 3.15E−03 | 0.66 |
| TMEM23 | 730534 | 3.19E−03 | 0.77 |
| KIAA0789 | 33621 | 3.23E−03 | 0.43 |
| GSTM2 | 664233 | 3.33E−03 | 0.49 |
| IMAGE:135125 | 135125 | 3.33E−03 | 0.62 |
| IGKV1-5 | 155345 | 3.35E−03 | 0.62 |
| KLF1 | 208991 | 3.37E−03 | 0.43 |
| SYT7 | 177827 | 3.38E−03 | 0.65 |
| IMAGE:667353 | 667353 | 3.59E−03 | 0.76 |
| Transcribed | | | |
| locus | | | |
| ZNF76 | 745003 | 3.60E−03 | 0.69 |
| IMAGE:731685 | 731685 | 3.66E−03 | 0.72 |
| Transcribed | | | |
| locus | | | |
| MAP7 | 79729 | 3.98E−03 | 0.49 |
| DOK5 | 25664 | 4.00E−03 | 0.65 |
| PDGFRL | 139242 | 4.04E−03 | 0.58 |
| IMAGE:666140 | 666140 | 4.06E−03 | 0.65 |
| Transcribed | | | |
| locus, weakly | | | |
| similar to | | | |
| XP_498467.1 | | | |
| PREDICTED: | | | |
| hypothetical | | | |
| protein | | | |
| XP_498467 | | | |
| [Homo | | | |
| sapiens] | | | |
| ETV5 | 270549 | 4.26E−03 | 0.63 |
| C6orf176 | 173200 | 4.34E−03 | 0.55 |
| PPIL4 | 364777 | 4.41E−03 | 0.41 |
| TUSC1 | 740381 | 4.50E−03 | 0.61 |
| SOD3 | 795309 | 4.50E−03 | 0.74 |
| C14orf140 | 731423 | 4.52E−03 | 0.52 |
| ARF4L | 49888 | 4.53E−03 | 0.55 |
| RGL2 | 741891 | 4.57E−03 | 0.81 |
| BCL2 | 232714 | 4.61E−03 | 0.68 |
| IMAGE:743290 | 743290 | 4.66E−03 | 0.64 |
| Transcribed | | | |
| locus | | | |
| CAV1 | 133531 | 4.69E−03 | 0.67 |
| C6orf85 | 665379 | 4.74E−03 | 0.71 |
| PIGV | 669319 | 4.80E−03 | 0.59 |
| TIMP2 | 258127 | 5.06E−03 | 0.66 |
| NR2F6 | 1238492 | 5.07E−03 | 0.70 |
| IGF1R | 682555 | 5.09E−03 | 0.65 |
| MFAP3L | 726821 | 5.09E−03 | 0.67 |
| PLXNB2 | 1420676 | 5.10E−03 | 0.58 |
| SATB1 | 364510 | 5.10E−03 | 0.82 |
| DEXI | 668186 | 5.31E−03 | 0.59 |
| VCL | 44193 | 5.31E−03 | 0.63 |
| HSPC159 | 365045 | 5.58E−03 | 0.60 |
| FLJ22386 | 32917 | 6.06E−03 | 0.63 |
| PLCG1 | 1174287 | 6.11E−03 | 0.74 |
| DAP | 725371 | 6.20E−03 | 0.67 |
| DUSP3 | 119772 | 6.35E−03 | 0.62 |
| EIF2C4 | 730834 | 6.43E−03 | 0.75 |
| IMAGE:252382 | 252382 | 6.53E−03 | 0.59 |
| C10orf6 | 179214 | 6.56E−03 | 0.80 |
| BAK1 | 1288183 | 6.60E−03 | 0.64 |
| GPD1 | 628418 | 6.66E−03 | 0.67 |
| GNAS | 382791 | 6.71E−03 | 0.67 |
| BIN1 | 2384812 | 6.83E−03 | 0.59 |
| KIAA1277 | 364822 | 6.86E−03 | 0.68 |
| IMAGE:726513 | 726513 | 6.95E−03 | 0.70 |
| Similar to | | | |
| 40S | | | |
| ribosomal | | | |
| protein S3 | | | |
| CA11 | 282587 | 7.06E−03 | 0.68 |
| LEMD1 | 731047 | 7.18E−03 | 0.65 |
| ASGR1 | 25883 | 7.35E−03 | 0.56 |
| LOC286170 | 134858 | 7.42E−03 | 0.64 |
| SLC17A2 | 207920 | 7.47E−03 | 0.49 |
| FLJ37587 | 668476 | 7.51E−03 | 0.69 |
| FLJ21159 | 251147 | 7.60E−03 | 0.78 |
| MLPH | 667259 | 7.71E−03 | 0.49 |
| SLIT1 | 38403 | 7.80E−03 | 0.70 |
| ZNF398 | 365311 | 8.35E−03 | 0.60 |
| FBXL16 | 178908 | 8.83E−03 | 0.74 |
| CAV1 | 1911930 | 8.91E−03 | 0.61 |
| TTC7B | 135379 | 9.25E−03 | 0.78 |
| IMAGE:681875 | 681875 | 9.30E−03 | 0.66 |
| GATA2 | 149809 | 9.39E−03 | 0.79 |
| RAP1GA1 | 971276 | 9.50E−03 | 0.66 |
| IMAGE:687667 | 687667 | 9.52E−03 | 0.59 |
| CDNA | | | |
| clone | | | |
| IMAGE:4811759, | | | |
| partial | | | |
| cds | | | |
| TEX13A | 738558 | 9.75E−03 | 0.67 |
| DKFZp566O084 | 136952 | 9.79E−03 | 0.60 |
| KIAA1958 | 687287 | 9.87E−03 | 0.65 |
| ZFOC1 | 744606 | 9.99E−03 | 0.57 |
| DKFZp434M202 | 743118 | 1.02E−02 | 0.63 |
| FLJ13491 | 364352 | 1.02E−02 | 0.72 |
| PPP2CB | 667375 | 1.02E−02 | 0.56 |
| IMAGE:726647 | 726647 | 1.04E−02 | 0.74 |
| Transcribed | | | |
| locus | | | |
| KCNK5 | 134978 | 1.04E−02 | 0.70 |
| FLJ44005 | 726675 | 1.06E−02 | 0.66 |
| DMD | 743394 | 1.07E−02 | 0.61 |
| IMAGE:738424 | 738424 | 1.12E−02 | 0.72 |
| PINK1 | 729929 | 1.14E−02 | 0.66 |
| DNALI1 | 782688 | 1.16E−02 | 0.53 |
| IMAGE:726782 | 726782 | 1.20E−02 | 0.64 |
| Transcribed | | | |
| locus | | | |
| HT007 | 135303 | 1.22E−02 | 0.79 |
| C9orf10OS | 742695 | 1.30E−02 | 0.60 |
| IMAGE:363955 | 363955 | 1.31E−02 | 0.62 |
| Transcribed | | | |
| locus | | | |
| FLJ37034 | 731348 | 1.32E−02 | 0.70 |
| KRT17 | 366889 | 1.33E−02 | 0.63 |
| TSG101 | 194350 | 1.34E−02 | 0.66 |
| MPO | 436554 | 1.38E−02 | 0.80 |
| IMAGE:668536 | 668536 | 1.39E−02 | 0.47 |
| CDNA | | | |
| FLJ41685 fis, | | | |
| clone | | | |
| HCASM2006338 | | | |
| TETRAN | 725340 | 1.41E−02 | 0.72 |
| IMAGE:174861 | 174861 | 1.46E−02 | 0.69 |
| Transcribed | | | |
| locus | | | |
| IMAGE:1859532 | 1859532 | 1.47E−02 | 0.82 |
| CIRH1A | 261500 | 1.48E−02 | 0.64 |
| UQCRC1 | 714414 | 1.53E−02 | 0.69 |
| LOC286272 | 667361 | 1.54E−02 | 0.70 |
| IMAGE:742739 | 742739 | 1.55E−02 | 0.65 |
| CDNA | | | |
| FLJ12931 fis, | | | |
| clone | | | |
| NT2RP2004861 | | | |
| IMAGE:364741 | 364741 | 1.58E−02 | 0.68 |
| C20orf19 | 366032 | 1.60E−02 | 0.66 |
| LOC147650 | 729510 | 1.61E−02 | 0.70 |
| TSC | 745490 | 1.62E−02 | 0.66 |
| AURKC | 731021 | 1.63E−02 | 0.77 |
| HIS1 | 342551 | 1.65E−02 | 0.65 |
| PCGF4 | 740457 | 1.67E−02 | 0.68 |
| ZNF512 | 726596 | 1.69E−02 | 0.77 |
| LILRA1 | 2032639 | 1.71E−02 | 0.59 |
| IMAGE:136121 | 136121 | 1.73E−02 | 0.73 |
| CDNA | | | |
| FLJ10247 fis, | | | |
| clone | | | |
| HEMBB1000705 | | | |
| IMAGE:745072 | 745072 | 1.78E−02 | 0.72 |
| Transcribed | | | |
| locus | | | |
| DEFB1 | 665086 | 1.80E−02 | 0.52 |
| IMAGE:738966 | 738966 | 1.82E−02 | 0.52 |
| Transcribed | | | |
| locus | | | |
| NAT5 | 731073 | 1.84E−02 | 0.65 |
| LARS | 135407 | 1.87E−02 | 0.65 |
| FBXO46 | 471664 | 1.96E−02 | 0.60 |
| ECM1 | 301122 | 1.96E−02 | 0.39 |
| HDAC9 | 738506 | 1.97E−02 | 0.65 |
| PSG3 | 136747 | 1.99E−02 | 0.59 |
| SEMA4B | 714437 | 1.99E−02 | 0.73 |
| IMAGE:485104 | 485104 | 2.05E−02 | 0.63 |
| Transcribed | | | |
| locus | | | |
| ITIH5 | 179733 | 2.06E−02 | 0.60 |
| LEF1 | 713913 | 2.07E−02 | 0.68 |
| LOC285961 | 742542 | 2.12E−02 | 0.65 |
| MED19 | 743760 | 2.15E−02 | 0.81 |
| CDC42EP2 | 26651 | 2.15E−02 | 0.68 |
| DPF2 | 743519 | 2.18E−02 | 0.79 |
| AKR1C1 | 196992 | 2.21E−02 | 0.38 |
| IMAGE:665392 | 665392 | 2.23E−02 | 0.81 |
| Hypothetical | | | |
| LOC388790 | | | |
| MRPL45 | 364717 | 2.25E−02 | 0.64 |
| HMG20A | 731277 | 2.28E−02 | 0.72 |
| PKNOX1 | 667067 | 2.32E−02 | 0.72 |
| ACTR1B | 136868 | 2.41E−02 | 0.69 |
| DLX5 | 564878 | 2.45E−02 | 0.57 |
| GGN | 743245 | 2.47E−02 | 0.80 |
| ZNF580 | 668089 | 2.48E−02 | 0.76 |
| CREB3L2 | 136399 | 2.49E−02 | 0.52 |
| IMAGE:383718 | 383718 | 2.52E−02 | 0.71 |
| SLC25A23 | 741954 | 2.58E−02 | 0.52 |
| RP4-622L5 | 742590 | 2.62E−02 | 0.85 |
| LCN2 | 544683 | 2.69E−02 | 0.68 |
| LOC56930 | 134976 | 2.70E−02 | 0.79 |
| FLJ45459 | 365589 | 2.71E−02 | 0.76 |
| SPATA12 | 730300 | 2.78E−02 | 0.67 |
| GALNT7 | 381854 | 2.83E−02 | 0.73 |
| MCC | 731305 | 2.84E−02 | 0.69 |
| BRMS1 | 2348237 | 2.86E−02 | 0.70 |
| FRMD4B | 669564 | 2.89E−02 | 0.63 |
| ERK8 | 714049 | 2.91E−02 | 0.67 |
| CDR2 | 366067 | 2.94E−02 | 0.76 |
| WIPI49 | 487148 | 3.02E−02 | 0.83 |
| SCEL | 668239 | 3.06E−02 | 0.72 |
| ABCB1 | 1837488 | 3.12E−02 | 0.62 |
| COL18A1 | 359202 | 3.12E−02 | 0.70 |
| MLANA | 266361 | 3.13E−02 | 0.70 |
| LOC283070 | 172785 | 3.14E−02 | 0.71 |
| FGF1 | 25895 | 3.23E−02 | 0.73 |
| PAX8 | 545475 | 3.24E−02 | 0.41 |
| KLHL9 | 501527 | 3.25E−02 | 0.72 |
| IMAGE:726981 | 726981 | 3.28E−02 | 0.73 |
| Transcribed | | | |
| locus, | | | |
| strongly | | | |
| similar to | | | |
| XP_526325.1 | | | |
| PREDICTED: | | | |
| similar to | | | |
| retinol | | | |
| binding | | | |
| protein 2, | | | |
| cellular; | | | |
| retinol- | | | |
| binding | | | |
| protein 2, | | | |
| cellular [Pan | | | |
| troglodytes] | | | |
| CDH3 | 359051 | 3.33E−02 | 0.77 |
| VTN | 230126 | 3.38E−02 | 0.74 |
| PARD3 | 724642 | 3.53E−02 | 0.72 |
| FLT4 | 668815 | 3.53E−02 | 0.68 |
| KIAA0515 | 687852 | 3.55E−02 | 0.71 |
| SIN3A | 26455 | 3.56E−02 | 0.68 |
| CITED2 | 286138 | 3.58E−02 | 0.74 |
| EBAG9 | 682507 | 3.58E−02 | 0.65 |
| FLJ31438 | 135539 | 3.61E−02 | 0.66 |
| HBB | 469549 | 3.63E−02 | 0.50 |
| ACADSB | 687953 | 3.76E−02 | 0.68 |
| KIAA1280 | 366085 | 3.80E−02 | 0.68 |
| MTUS1 | 744395 | 3.88E−02 | 0.60 |
| LOC146909 | 726353 | 3.91E−02 | 0.79 |
| BCAR1 | 324666 | 3.94E−02 | 0.77 |
| LOC339903 | 135352 | 3.97E−02 | 0.64 |
| MESP1 | 25865 | 4.05E−02 | 0.73 |
| S100A1 | 175772 | 4.11E−02 | 0.60 |
| DNCLI2 | 134671 | 4.18E−02 | 0.71 |
| S100A16 | 730699 | 4.40E−02 | 0.75 |
| IMAGE:22908 | 22908 | 4.56E−02 | 0.76 |
| Transcribed | | | |
| locus, weakly | | | |
| similar to | | | |
| NP_775735.1 | | | |
| I(3)mbt-like | | | |
| 4 [Homo | | | |
| sapiens] | | | |
| DPP4 | 343987 | 4.56E−02 | 0.72 |
| NCAM1 | 366842 | 4.62E−02 | 0.80 |
| MAPK3 | 323438 | 4.64E−02 | 0.74 |
| LRRC28 | 26519 | 4.68E−02 | 0.57 |
| PBXIP1 | 366042 | 4.76E−02 | 0.76 |
| IMAGE:727067 | 727067 | 4.76E−02 | 0.77 |
| Similar to | | | |
| hypothetical | | | |
| protein | | | |
| SB153 | | | |
| isoform 1 | | | |
| EMX2 | 365121 | 4.96E−02 | 0.74 |
| NOTCH1 | 359461 | 4.99E−02 | 0.65 |

TABLE 4

| | AT | Over | |
|---|---|---|---|
| name | cloid | p-value | fold |
| PTK2B | 43541 | 2.72E−05 | 2.23 |
| ARHGEF6 | 687990 | 4.43E−05 | 2.00 |
| IMAGE:365990 | 365990 | 1.34E−04 | 1.32 |
| STK17A | 562904 | 2.09E−04 | 3.60 |
| HLA-DPB1 | 725548 | 4.45E−04 | 4.47 |
| SIAT4B | 365877 | 4.46E−04 | 1.32 |
| FCHO1 | 742763 | 5.60E−04 | 2.71 |
| GIMAP5 | 180259 | 5.64E−04 | 3.22 |
| CCL13 | 2449149 | 6.35E−04 | 3.15 |
| IMAGE:1089025 | 1089025 | 7.30E−04 | 12.77 |
| IFITM3 | 713623 | 7.87E−04 | 2.21 |
| BTG1 | 382760 | 9.03E−04 | 2.34 |
| IL10RA | 258747 | 9.17E−04 | 4.77 |
| FLJ10858 | 743961 | 9.41E−04 | 1.86 |
| STX7 | 174396 | 1.03E−03 | 7.61 |
| GNAI2 | 724306 | 1.06E−03 | 1.36 |
| IL13 | 1505308 | 1.09E−03 | 1.66 |
| SALL4 | 726454 | 1.11E−03 | 11.25 |
| HCLS1 | 665452 | 1.19E−03 | 5.103473 |
| IMAGE:665833 | 665833 | 1.28E−03 | 1.86 |
| CD38 | 1352408 | 1.34E−03 | 3.46 |
| MGAT3 | 731060 | 1.48E−03 | 2.02 |
| TNFRSF1B | 73703 | 1.49E−03 | 2.56 |
| SULT1E1 | 207926 | 1.52E−03 | 1.23 |
| CERKL | 489326 | 1.70E−03 | 5.60 |
| CD8B1 | 1743279 | 2.38E−03 | 2.01 |
| IL2RB | 139073 | 2.50E−03 | 3.459251 |
| SLAMF1 | 1626951 | 2.54E−03 | 2.51 |
| CBL | 1578721 | 2.62E−03 | 3.37 |
| POU2F2 | 188393 | 2.66E−03 | 2.90 |
| RNASE4 | 156720 | 2.74E−03 | 1.84 |
| GATA4 | 781738 | 3.03E−03 | 3.80 |
| PDK1 | 1645668 | 3.07E−03 | 2.03 |
| SRGAP2 | 714493 | 3.13E−03 | 1.25 |
| HDAC1 | 1896337 | 3.50E−03 | 2.16 |
| GC | 195340 | 3.69E−03 | 4.97 |
| ACTB | 203166 | 3.97E−03 | 1.91 |
| F10 | 310519 | 4.05E−03 | 1.580546 |
| NBL1 | 503874 | 5.07E−03 | 6.38 |
| IMAGE:743722 | 743722 | 5.15E−03 | 3.83 |
| YWHAH | 324066 | 5.22E−03 | 1.45 |
| PDCD1LG2 | 738944 | 5.28E−03 | 2.78 |
| PLCG2 | 201467 | 5.28E−03 | 2.986551 |
| APOE | 1870594 | 5.58E−03 | 5.323048 |
| RUNX3 | 122874 | 5.87E−03 | 4.16 |
| GSS | 140405 | 6.10E−03 | 2.46 |
| FXYD1 | 204686 | 6.16E−03 | 1.62 |
| IMAGE:2497617 | 2497617 | 6.40E−03 | 1.77 |
| STAT2 | 2306096 | 6.71E−03 | 1.74 |
| CCL19 | 1707527 | 7.61E−03 | 34.27 |
| BAZ2B | 609631 | 7.62E−03 | 2.15 |
| CASP4 | 356960 | 8.00E−03 | 3.00 |
| CCR7 | 2345206 | 8.18E−03 | 2.62 |
| IFI30 | 740931 | 8.38E−03 | 4.70 |
| IMAGE:342256 | 342256 | 8.64E−03 | 21.04 |
| KLRK1 | 725473 | 9.04E−03 | 3.94 |
| IRF4 | 682207 | 9.24E−03 | 4.80 |
| TYMS | 2242054 | 9.47E−03 | 1.85 |
| S100A4 | 868577 | 9.52E−03 | 2.49 |
| IMAGE:727154 | 727154 | 9.72E−03 | 2.26 |
| Transcribed | | | |
| locus | | | |
| CASP7 | 279470 | 9.89E−03 | 1.99 |
| TOSO | 813174 | 9.90E−03 | 3.28 |
| FLJ12592 | 252489 | 1.06E−02 | 1.94 |
| LILRB4 | 2341829 | 1.06E−02 | 1.47 |
| CPXM2 | 729924 | 1.12E−02 | 2.72 |
| RUNX1 | 263251 | 1.12E−02 | 1.56 |
| UBE1L | 250883 | 1.13E−02 | 1.73 |
| IMAGE:738896 | 738896 | 1.14E−02 | 18.07 |
| NCK1 | 1326169 | 1.14E−02 | 5.22 |
| SEPT-05 | 448163 | 1.15E−02 | 18.97 |
| NFYA | 731648 | 1.17E−02 | 2.21 |
| IMAGE:2113771 | 2113771 | 1.21E−02 | 2.22 |
| HNRPA3 | 365349 | 1.21E−02 | 1.22 |
| WDR5 | 731023 | 1.24E−02 | 2.80 |
| MLL4 | 744980 | 1.29E−02 | 1.81 |
| NYREN18 | 668584 | 1.31E−02 | 2.21 |
| IMAGE:727496 | 727496 | 1.32E−02 | 6.10 |
| Transcribed | | | |
| locus | | | |
| IMAGE:132933 | 132933 | 1.33E−02 | 1.21 |
| LASP1 | 592598 | 1.39E−02 | 11.76 |
| IMAGE:46991 | 46991 | 1.40E−02 | 4.89 |
| Similar to | | | |
| breakpoint | | | |
| cluster region | | | |
| isoform 1 | | | |
| BID | 128065 | 1.41E−02 | 2.16 |
| SMARCA5 | 730037 | 1.48E−02 | 2.82 |
| CD5 | 356841 | 1.49E−02 | 2.31 |
| PIK3CA | 2238108 | 1.51E−02 | 11.79 |
| CASP3 | 823680 | 1.55E−02 | 2.51 |
| DAXX | 292042 | 1.56E−02 | 1.91 |
| IMAGE:504539 | 504539 | 1.56E−02 | 1.91 |
| RGS19 | 485803 | 1.61E−02 | 3.13 |
| LEFTY2 | 340657 | 1.62E−02 | 3.621832 |
| MGP | 590264 | 1.76E−02 | 2.58 |
| GADD45GIP1 | 1084386 | 1.79E−02 | 2.82 |
| IMAGE:742853 | 742853 | 1.81E−02 | 9.42 |
| SEMA4D | 210587 | 1.81E−02 | 3.62 |
| HMGB2 | 884365 | 1.83E−02 | 1.87 |
| NOMO1 | 134615 | 1.84E−02 | 1.70 |
| IMAGE:740927 | 740927 | 1.91E−02 | 1.46 |
| FABP5 | 1088781 | 2.02E−02 | 14.00 |
| KLRB1 | 2091591 | 2.03E−02 | 4.00 |
| G3BP | 667239 | 2.08E−02 | 4.05 |
| HPS4 | 727430 | 2.09E−02 | 2.04 |
| ZNF251 | 486401 | 2.15E−02 | 14.82 |
| TRAF5 | 1286238 | 2.15E−02 | 2.90 |
| MAPKAPK2 | 742577 | 2.17E−02 | 1.56 |
| STAT6 | 1916307 | 2.19E−02 | 1.79 |
| IL2RA | 3054031 | 2.20E−02 | 1.81 |
| APOL3 | 366289 | 2.26E−02 | 3.52 |
| DGKQ | 366233 | 2.31E−02 | 1.84 |
| MLL | 80688 | 2.32E−02 | 1.78 |
| APOL1 | 665632 | 2.35E−02 | 4.01 |
| IRAK1 | 379200 | 2.41E−02 | 1.64 |
| TIMP1 | 162246 | 2.47E−02 | 4.54 |
| FLJ27523 | 743246 | 2.48E−02 | 12.16 |
| CRYZ | 486935 | 2.48E−02 | 1.46 |
| GRM3 | 287843 | 2.56E−02 | 4.69 |
| MYO5A | 365755 | 2.62E−02 | 1.44 |
| BST2 | 811024 | 2.65E−02 | 2.64 |
| CKLFSF3 | 489249 | 2.70E−02 | 1.54651 |
| CCL5 | 840753 | 2.74E−02 | 1.63 |
| IMAGE:731689 | 731689 | 2.74E−02 | 1.25 |
| Transcribed | | | |
| locus | | | |
| BAI3 | 50491 | 2.77E−02 | 4.54 |
| LILRB3 | 1422194 | 2.78E−02 | 2.77 |
| ATR | 1251197 | 2.79E−02 | 2.31 |
| PDCD1LG1 | 1942634 | 2.81E−02 | 2.44 |
| LOC440712 | 725268 | 2.85E−02 | 2.10 |
| DCN | 666410 | 2.87E−02 | 1.70 |
| IMAGE:743415 | 743415 | 2.88E−02 | 2.30 |
| Transcribed | | | |
| locus | | | |
| IKBKE | 364448 | 2.91E−02 | 1.49 |
| NCB5OR | 743367 | 3.01E−02 | 1.99 |
| MAP2 | 1287638 | 3.19E−02 | 1.44 |
| DDR2 | 668442 | 3.20E−02 | 1.17 |
| SYT1 | 28511 | 3.26E−02 | 1.61 |
| PPP1CA | 1422791 | 3.29E−02 | 1.43 |
| IMAGE:743224 | 743224 | 3.34E−02 | 1.76 |
| Transcribed | | | |
| locus, | | | |
| strongly | | | |
| similar to | | | |
| XP_518162.1 | | | |
| PREDICTED: | | | |
| similar to | | | |
| Zinc finger | | | |
| protein 62 | | | |
| homolog | | | |
| (Zfp-62) | | | |
| (ZT3) [Pan | | | |
| troglodytes] | | | |
| VIL2 | 124701 | 3.35E−02 | 1.87 |
| CECR1 | 725612 | 3.36E−02 | 1.33 |
| HN1 | 471568 | 3.55E−02 | 2.25 |
| ICAM3 | 156183 | 3.60E−02 | 3.73 |
| MUC1 | 840687 | 3.66E−02 | 2.25 |
| IMAGE:731751 | 731751 | 3.66E−02 | 1.33 |
| Transcribed | | | |
| locus | | | |
| S100A12 | 1705397 | 3.67E−02 | 2.13 |
| PTPNS1 | 26061 | 3.87E−02 | 1.894355 |
| NRP1 | 666061 | 3.89E−02 | 1.38 |
| NEK6 | 725345 | 3.92E−02 | 2.27 |
| PREB | 740347 | 3.94E−02 | 4.022672 |
| SFN | 346610 | 4.09E−02 | 1.65 |
| FOLR1 | 131839 | 4.11E−02 | 1.47 |
| IFNAR2 | 123950 | 4.16E−02 | 3.29 |
| BCAP29 | 2336358 | 4.24E−02 | 2.02 |
| TNIP1 | 135801 | 4.27E−02 | 1.59 |
| TOP1 | 666425 | 4.28E−02 | 1.24 |
| ETV4 | 809959 | 4.42E−02 | 1.23 |
| FGF4 | 1550616 | 4.53E−02 | 1.40 |
| WAS | 2148946 | 4.57E−02 | 3.27 |
| KLRD1 | 2728204 | 4.58E−02 | 1.80 |
| SNX8 | 366585 | 4.61E−02 | 1.57 |
| KPNA2 | 667727 | 4.62E−02 | 1.44 |
| PMS2 | 116906 | 4.68E−02 | 4.11 |
| SLAMF8 | 288807 | 4.80E−02 | 3.22 |
| CXCL14 | 345034 | 4.82E−02 | 4.25 |
| DNMT1 | 768241 | 4.91E−02 | 1.71 |
| PARP9 | 667440 | 4.91E−02 | 1.75 |

TABLE 5

| | GD | Under | |
|---|---|---|---|
| name | cloid | p-value | fold |
| DOK5 | 25664 | 2.9346E−09 | 0.57 |
| LPL | 868169 | 1.1605E−06 | 0.55 |
| ARHGAP10 | 731380 | 4.7117E−06 | 0.65 |
| CLGN | 1049033 | 9.1105E−06 | 0.61 |
| DECR2 | 730942 | 1.5383E−05 | 0.61 |
| IMAGE:731616 | 731616 | 1.852E−05 | 0.59 |
| MRNA; | | | |
| cDNA | | | |
| DKFZp686A102 | | | |
| (from | | | |
| clone | | | |
| DKFZp686A102) | | | |
| IQCF3 | 726578 | 2.3435E−05 | 0.71 |
| PSAT1 | 366388 | 3.1255E−05 | 0.79 |
| CHRDL2 | 485872 | 3.6361E−05 | 0.71 |
| SERPINB5 | 1662274 | 0.00015833 | 0.66 |
| ICOSLG | 2074228 | 0.00018032 | 0.72 |
| OBSCN | 730926 | 0.00018977 | 0.81 |
| ANKRD20B | 745106 | 0.00019641 | 0.57 |
| AURKC | 731021 | 0.00020316 | 0.57 |
| SLC17A2 | 207920 | 0.00021513 | 0.47 |
| CLECSF12 | 258865 | 0.00041555 | 0.34 |
| VEGFC | 503189 | 0.00048933 | 0.59 |
| TSC | 745490 | 0.00057708 | 0.51 |
| LAMB3 | 1103402 | 0.00060694 | 0.61 |
| CAV1 | 1911930 | 0.00067972 | 0.61 |
| MCC | 731305 | 0.00108212 | 0.55 |
| NOLA2 | 173296 | 0.00116175 | 0.69 |
| ZNF651 | 669181 | 0.00116222 | 0.76 |
| IMAGE:727491 | 727491 | 0.00121836 | 0.45 |
| MX1 | 713879 | 0.00133899 | 0.64 |
| NOTCH1 | 359461 | 0.00137335 | 0.72 |
| CITED1 | 265558 | 0.00146133 | 0.52 |
| IMAGE:687667 | 687667 | 0.00149013 | 0.5 |
| CDNA | | | |
| clone | | | |
| IMAGE:4811759, | | | |
| partial | | | |
| cds | | | |
| SCEL | 668239 | 0.00152752 | 0.65 |
| IMAGE:232366 | 232366 | 0.00210682 | 0.61 |
| CDNA | | | |
| FLJ37672 | | | |
| fis, clone | | | |
| BRHIP2012059 | | | |
| C21orf84 | 730814 | 0.0021117 | 0.67 |
| AOC3 | 484535 | 0.00216625 | 0.7 |
| IMP-2 | 743774 | 0.00217549 | 0.52 |
| PRKG1 | 667587 | 0.00219736 | 0.67 |
| DEFB1 | 665086 | 0.00251628 | 0.42 |
| KRT17 | 366889 | 0.00252804 | 0.71 |
| SAP30L | 723950 | 0.00263794 | 0.78 |
| PANK1 | 256177 | 0.00274492 | 0.75 |
| IMAGE:1859532 | 1859532 | 0.00278248 | 0.77 |
| C20orf18 | 665433 | 0.00297457 | 0.62 |
| PIP | 985457 | 0.00350912 | 0.8 |
| DLX5 | 564878 | 0.00369818 | 0.66 |
| ST5 | 376356 | 0.00372025 | 0.79 |
| MAPRE2 | 383868 | 0.00390703 | 0.64 |
| FBXO46 | 471664 | 0.00493537 | 0.6 |
| PARD3 | 724642 | 0.00552617 | 0.73 |
| IMAGE:23817 | 23817 | 0.00606222 | 0.5 |
| CDNA | | | |
| FLJ12202 | | | |
| fis, clone | | | |
| MAMMA1000908 | | | |
| DCXR | 724596 | 0.00613681 | 0.58 |
| LRRIQ2 | 487152 | 0.00618225 | 0.83 |
| EPRS | 669549 | 0.00624669 | 0.71 |
| IGKV1-5 | 155345 | 0.00626237 | 0.63 |
| CXCL13 | 347362 | 0.00699423 | 0.83 |
| TOP1 | 666425 | 0.00725755 | 0.74 |
| ETV1 | 24541 | 0.00727553 | 0.68 |
| RFPL1 | 290265 | 0.00745468 | 0.69 |
| FLJ25067 | 667252 | 0.0075477 | 0.72 |
| MGC45840 | 725231 | 0.00862765 | 0.73 |
| DPP4 | 343987 | 0.00891505 | 0.55 |
| RBBP4 | 705147 | 0.00896277 | 0.77 |
| NR2F6 | 1238492 | 0.00901903 | 0.7 |
| IMAGE:745072 | 745072 | 0.00950104 | 0.59 |
| Transcribed | | | |
| locus | | | |
| LCN2 | 544683 | 0.01012252 | 0.63 |
| IL2RA | 3054031 | 0.01017643 | 0.84 |
| IMAGE:744604 | 744604 | 0.01062584 | 0.59 |
| NAT5 | 731073 | 0.0108843 | 0.67 |
| EPHA4 | 2237263 | 0.01141372 | 0.58 |
| CCL28 | 136919 | 0.01174159 | 0.83 |
| PPAP2C | 486447 | 0.01184402 | 0.73 |
| TPM2 | 740620 | 0.01184508 | 0.68 |
| PLXNB2 | 1420676 | 0.0119905 | 0.72 |
| FOXO1A | 151247 | 0.01279281 | 0.8 |
| TEX13A | 738558 | 0.01337572 | 0.74 |
| AD023 | 731645 | 0.01337901 | 0.68 |
| TPCN1 | 179288 | 0.01386141 | 0.67 |
| IMAGE:667683 | 667683 | 0.01420475 | 0.8 |
| Full- | | | |
| length cDNA | | | |
| clone | | | |
| CS0DK008YI09 | | | |
| of HeLa | | | |
| cells Cot 25- | | | |
| normalized | | | |
| of Homo | | | |
| sapiens | | | |
| (human) | | | |
| IMAGE:135125 | 135125 | 0.01424832 | 0.65 |
| SPINT1 | 723914 | 0.0145432 | 0.83 |
| CENTD1 | 666110 | 0.01458746 | 0.66 |
| GALNT7 | 381854 | 0.01458803 | 0.65 |
| KIAA1754L | 365056 | 0.015234 | 0.76 |
| DJ462O23.2 | 738970 | 0.01526675 | 0.82 |
| PML | 724554 | 0.01579712 | 0.84 |
| ECM1 | 301122 | 0.01646653 | 0.37 |
| IMAGE:382521 | 382521 | 0.01686383 | 0.67 |
| CASP5 | 341763 | 0.01711585 | 0.67 |
| PSG3 | 136747 | 0.01796368 | 0.64 |
| MRPL45 | 364717 | 0.01798173 | 0.59 |
| IMAGE:484577 | 484577 | 0.01809095 | 0.58 |
| Transcribed | | | |
| locus, | | | |
| weakly | | | |
| similar to | | | |
| NP_000991.1 | | | |
| ribosomal | | | |
| protein L39; | | | |
| 60S | | | |
| ribosomal | | | |
| protein L39 | | | |
| [Homo | | | |
| sapiens] | | | |
| FLJ22471 | 668510 | 0.0182197 | 0.6 |
| KLRD1 | 145696 | 0.01870053 | 0.72 |
| CLDN1 | 594279 | 0.01895465 | 0.39 |
| TETRAN | 725340 | 0.01932623 | 0.72 |
| RAB8A | 712118 | 0.01937338 | 0.88 |
| HP | 82687 | 0.01968016 | 0.79 |
| LOC120224 | 252291 | 0.01972775 | 0.61 |
| LOC80298 | 666551 | 0.01993545 | 0.7 |
| C7orf27 | 713859 | 0.02018391 | 0.92 |
| CDH3 | 359051 | 0.02040993 | 0.64 |
| CGI-128 | 786662 | 0.02095299 | 0.73 |
| LCN2 | 741497 | 0.02142784 | 0.4 |
| IMAGE:731298 | 731298 | 0.02244117 | 0.6 |
| CDNA | | | |
| FLJ42482 | | | |
| fis, clone | | | |
| BRACE2032 | | | |
| MKKS | 729957 | 0.02271256 | 0.64 |
| IMAGE:743903 | 743903 | 0.02274312 | 0.59 |
| Homo | | | |
| sapiens, | | | |
| clone | | | |
| IMAGE:5266541, | | | |
| mRNA | | | |
| IGF1R | 682555 | 0.02318362 | 0.79 |
| GCK | 30981 | 0.02333357 | 0.76 |
| LATS2 | 666778 | 0.02359943 | 0.75 |
| LARS | 135407 | 0.02385825 | 0.68 |
| IMAGE:742919 | 742919 | 0.02420455 | 0.84 |
| Transcribed | | | |
| locus | | | |
| IMAGE:378458 | 378458 | 0.02425923 | 0.78 |
| IMAGE:743579 | 743579 | 0.02441628 | 0.71 |
| TIMP2 | 258127 | 0.02458546 | 0.71 |
| GLG1 | 365613 | 0.02464356 | 0.83 |
| CCNE1 | 357807 | 0.02635602 | 0.75 |
| IMAGE:383528 | 383528 | 0.02656958 | 0.36 |
| Transcribed | | | |
| locus, | | | |
| weakly | | | |
| similar to | | | |
| XP_375099.1 | | | |
| PREDICTED: | | | |
| similar to | | | |
| hypothetical | | | |
| protein | | | |
| FLJ25224 | | | |
| [Homo | | | |
| sapiens] | | | |
| ALDH1A3 | 486189 | 0.0273157 | 0.69 |
| KIAA1196 | 738938 | 0.02776484 | 0.76 |
| AQP4 | 279172 | 0.02819313 | 0.69 |
| IMAGE:665649 | 665649 | 0.02902427 | 0.72 |
| Transcribed | | | |
| locus | | | |
| ALCAM | 172828 | 0.02935177 | 0.72 |
| IMAGE:738332 | 738332 | 0.02979191 | 0.53 |
| Transcribed | | | |
| locus | | | |
| APLP2 | 549054 | 0.03108305 | 0.8 |
| C9orf10OS | 742695 | 0.03165276 | 0.77 |
| IMAGE:213529 | 213529 | 0.03175448 | 0.88 |
| Transcribed | | | |
| locus | | | |
| SOSTDC1 | 667048 | 0.03195182 | 0.8 |
| GCAT | 307094 | 0.03222091 | 0.87 |
| MPO | 436554 | 0.03392843 | 0.73 |
| CCND1 | 324079 | 0.03432325 | 0.65 |
| CD47 | 365526 | 0.03494508 | 0.84 |
| MRPS11 | 471574 | 0.03589496 | 0.68 |
| PEMT | 742580 | 0.03618273 | 0.71 |
| PKIG | 666277 | 0.03628293 | 0.64 |
| BRMS1 | 2348237 | 0.0374802 | 0.7 |
| FLJ35630 | 485652 | 0.03841929 | 0.65 |
| ZFOC1 | 744606 | 0.03860826 | 0.74 |
| LOC51760 | 52226 | 0.03910408 | 0.76 |
| C22orf16 | 236119 | 0.03972491 | 0.75 |
| IMAGE:730924 | 730924 | 0.04130682 | 0.86 |
| Transcribed | | | |
| locus | | | |
| C6orf176 | 173200 | 0.04189653 | 0.75 |
| HIS1 | 342551 | 0.04212231 | 0.63 |
| PLA2G6 | 744087 | 0.04281272 | 0.8 |
| CXCL14 | 345034 | 0.04431193 | 0.53 |
| PPP2R4 | 731590 | 0.04445623 | 0.79 |
| DKFZp434M202 | 743118 | 0.04619395 | 0.77 |
| CIRH1A | 261500 | 0.04708019 | 0.76 |
| IMAGE:136223 | 136223 | 0.04869167 | 0.77 |
| IMAGE:668536 | 668536 | 0.04890577 | 0.58 |
| CDNA | | | |
| FLJ41685 | | | |
| fis, clone | | | |
| HCASM2006338 | | | |
| TM7SF3 | 666928 | 0.04909454 | 0.79 |
| TCEA2 | 730149 | 0.04988725 | 0.66 |

TABLE 6

| | GD | Over | |
|---|---|---|---|
| name | cloid | p-value | fold |
| ARHGEF6 | 687990 | 1.40E−02 | 1.46 |
| FLJ10858 | 743961 | 2.05E−02 | 1.28 |
| GPR124 | 486493 | 2.48E−02 | 1.47 |
| IMAGE:379279 | 379279 | 2.61E−02 | 1.18 |
| RIPK3 | 667313 | 4.78E−02 | 1.35 |

TABLE 7

| | FTA-a | Under | |
|---|---|---|---|
| name | cloid | p-value | fold |
| BRMS1 | 2348237 | 1.2E−07 | 0.66 |
| TTC7B | 135379 | 2.4E−07 | 0.63 |
| FBXO10 | 743851 | 3.4E−07 | 0.68 |
| ZNF217 | 1712992 | 3.8E−07 | 0.71 |
| LOC90110 | 376699 | 5.4E−07 | 0.71 |
| ATR | 1251197 | 5.9E−07 | 0.75 |
| PLCG1 | 1174287 | 6.4E−07 | 0.58 |
| CACNB2 | 173841 | 9.4E−07 | 0.58 |
| VDAC1 | 486221 | 1.2E−06 | 0.60 |
| CDKN1B | 265345 | 1.8E−06 | 0.71 |
| LOC91526 | 666564 | 2E−06 | 0.68 |
| IMAGE:252382 | 252382 | 3.6E−06 | 0.80 |
| C1QBP | 173371 | 5.4E−06 | 0.68 |
| NAT5 | 731073 | 5.6E−06 | 0.68 |
| NR2F1 | 253386 | 5.9E−06 | 0.67 |
| SPI1 | 1285305 | 7.4E−06 | 0.70 |
| BNIP3 | 342700 | 1E−05 | 0.69 |
| C10orf6 | 179214 | 1.1E−05 | 0.75 |
| IL2RA | 3054031 | 1.3E−05 | 0.70 |
| TFRC | 359797 | 1.6E−05 | 0.66 |
| ALDH1A3 | 486189 | 1.9E−05 | 0.62 |
| CLECSF12 | 258865 | 1.9E−05 | 0.43 |
| C6orf216 | 731742 | 2.1E−05 | 0.71 |
| NRP1 | 666061 | 2.2E−05 | 0.68 |
| ZNF550 | 135705 | 2.3E−05 | 0.66 |
| IMAGE:745072 | 745072 | 2.4E−05 | 0.62 |
| Transcribed | | | |
| locus | | | |
| GLRX2 | 731044 | 3.1E−05 | 0.74 |
| IMAGE:731616 | 731616 | 3.5E−05 | 0.56 |
| MRNA; | | | |
| cDNA | | | |
| DKFZp686A102 | | | |
| (from | | | |
| clone | | | |
| DKFZp686A102) | | | |
| WAS | 2148946 | 4.2E−05 | 0.61 |
| RPS6KA5 | 258966 | 5.3E−05 | 0.72 |
| IMAGE:504539 | 504539 | 5.4E−05 | 0.70 |
| MDH1 | 725188 | 5.4E−05 | 0.73 |
| IMAGE:213529 | 213529 | 5.5E−05 | 0.71 |
| Transcribed | | | |
| locus | | | |
| IMAGE:742919 | 742919 | 8.2E−05 | 0.82 |
| Transcribed | | | |
| locus | | | |
| IMAGE:682585 | 682585 | 9.5E−05 | 0.72 |
| HSPH1 | 666554 | 0.00011 | 0.71 |
| RAE1 | 825224 | 0.00011 | 0.79 |
| CLGN | 1049033 | 0.00012 | 0.67 |
| HSPC159 | 365045 | 0.00013 | 0.73 |
| IMAGE:742837 | 742837 | 0.00014 | 0.67 |
| Transcribed | | | |
| locus | | | |
| KIAA0427 | 127507 | 0.00014 | 0.78 |
| ANKRD32 | 725198 | 0.00015 | 0.71 |
| PMS2 | 116906 | 0.00016 | 0.61 |
| MORF4L1 | 135875 | 0.00018 | 0.78 |
| TERF1 | 135773 | 0.00019 | 0.74 |
| CDKL1 | 486282 | 0.00021 | 0.74 |
| DPP4 | 343987 | 0.00021 | 0.52 |
| IMAGE:232366 | 232366 | 0.00022 | 0.71 |
| CDNA | | | |
| FLJ37672 | | | |
| fis, | | | |
| clone | | | |
| BRHIP2012059 | | | |
| RAD51C | 80162 | 0.00023 | 0.75 |
| COX7B | 566862 | 0.00026 | 0.64 |
| IMAGE:724544 | 724544 | 0.00027 | 0.74 |
| Similar to | | | |
| 40S | | | |
| ribosomal | | | |
| protein | | | |
| S25 | | | |
| SLC36A3 | 731115 | 0.00027 | 0.77 |
| CKS2 | 725454 | 0.00028 | 0.68 |
| TOSO | 813174 | 0.00032 | 0.69 |
| LEFTY2 | 340657 | 0.00033 | 0.68 |
| APOD | 838611 | 0.00034 | 0.37 |
| TMEM23 | 730534 | 0.00037 | 0.82 |
| FCHO1 | 742763 | 0.00038 | 0.74 |
| RPL27A | 178255 | 0.00039 | 0.57 |
| VPS26 | 687336 | 0.0004 | 0.78 |
| LOC285961 | 742542 | 0.00042 | 0.80 |
| IMAGE:667683 | 667683 | 0.00046 | 0.78 |
| Full- | | | |
| length | | | |
| cDNA | | | |
| clone | | | |
| CS0DK008YI09 | | | |
| of | | | |
| HeLa | | | |
| cells Cot | | | |
| 25- | | | |
| normalized | | | |
| of | | | |
| Homo | | | |
| sapiens | | | |
| (human) | | | |
| KLRK1 | 725473 | 0.00046 | 0.72 |
| IL2RB | 139073 | 0.0005 | 0.67 |
| MUTED | 731202 | 0.0006 | 0.72 |
| CLDN1 | 594279 | 0.00067 | 0.34 |
| IMAGE:727289 | 727289 | 0.00068 | 0.61 |
| Transcribed | | | |
| locus | | | |
| IMAGE:382423 | 382423 | 0.0007 | 0.65 |
| Transcribed | | | |
| locus | | | |
| IMAGE:724556 | 724556 | 0.00071 | 0.78 |
| Transcribed | | | |
| locus | | | |
| PCGF4 | 740457 | 0.00071 | 0.69 |
| IGSF10 | 682276 | 0.00072 | 0.76 |
| PGR1 | 666361 | 0.00075 | 0.81 |
| MX1 | 713879 | 0.0008 | 0.73 |
| DEK | 1016390 | 0.00086 | 0.77 |
| SIAT8F | 667110 | 0.00092 | 0.70 |
| NOTCH1 | 359461 | 0.00095 | 0.76 |
| DOK5 | 25664 | 0.00096 | 0.71 |
| DPF2 | 743519 | 0.00099 | 0.77 |
| OR7E38P | 486540 | 0.00099 | 0.82 |
| PDK1 | 1645668 | 0.00101 | 0.78 |
| IMAGE:744899 | 744899 | 0.00102 | 0.75 |
| Homo | | | |
| sapiens, | | | |
| clone | | | |
| IMAGE:4837072, | | | |
| mRNA | | | |
| STK17A | 562904 | 0.00103 | 0.70 |
| PRKCABP | 1174342 | 0.00108 | 0.82 |
| TIMP2 | 258127 | 0.00111 | 0.78 |
| STAT6 | 1916307 | 0.00118 | 0.82 |
| ATIC | 665693 | 0.00119 | 0.80 |
| LOC157697 | 714109 | 0.00119 | 0.79 |
| IMAGE:665649 | 665649 | 0.00119 | 0.75 |
| Transcribed | | | |
| locus | | | |
| MEF2C | 687270 | 0.0012 | 0.80 |
| PDCD1LG1 | 1942634 | 0.0012 | 0.74 |
| CD38 | 1352408 | 0.00123 | 0.80 |
| OSR1 | 364686 | 0.00127 | 0.76 |
| CD47 | 357442 | 0.0013 | 0.80 |
| MKKS | 729957 | 0.00136 | 0.80 |
| PSIP1 | 667598 | 0.00142 | 0.81 |
| SF3B1 | 739247 | 0.00146 | 0.79 |
| ANP32E | 382738 | 0.00146 | 0.69 |
| KLRD1 | 145696 | 0.00147 | 0.80 |
| CBL | 1578721 | 0.00147 | 0.73 |
| LCN2 | 544683 | 0.00153 | 0.54 |
| IMAGE:745512 | 745512 | 0.00168 | 0.79 |
| PTPN4 | 249311 | 0.0017 | 0.77 |
| RBBP4 | 705147 | 0.00175 | 0.77 |
| TIGD7 | 727482 | 0.0018 | 0.82 |
| CD47 | 365526 | 0.00182 | 0.80 |
| SPATA5L1 | 724884 | 0.00184 | 0.71 |
| UBE2E3 | 1575056 | 0.00188 | 0.77 |
| H17 | 739432 | 0.00193 | 0.77 |
| IMAGE:743224 | 743224 | 0.00197 | 0.72 |
| Transcribed | | | |
| locus, | | | |
| strongly | | | |
| similar to | | | |
| XP_518162.1 | | | |
| PREDICTED: | | | |
| similar to | | | |
| Zinc | | | |
| finger | | | |
| protein | | | |
| 62 | | | |
| homolog | | | |
| (Zfp-62) | | | |
| (ZT3) | | | |
| [Pan | | | |
| troglodytes] | | | |
| DNPEP | 730779 | 0.00205 | 0.77 |
| ANXA11 | 137238 | 0.00209 | 0.86 |
| ABCA8 | 284828 | 0.00213 | 0.70 |
| SOSTDC1 | 667048 | 0.00216 | 0.77 |
| SENP6 | 739237 | 0.00216 | 0.75 |
| LEMD1 | 731047 | 0.00224 | 0.67 |
| NCK1 | 1326169 | 0.00225 | 0.69 |
| NSUN4 | 364570 | 0.00231 | 0.82 |
| STX7 | 174396 | 0.00244 | 0.63 |
| DEFB1 | 665086 | 0.00244 | 0.40 |
| BAK1 | 235938 | 0.00245 | 0.74 |
| PDGFD | 666860 | 0.00245 | 0.80 |
| DMD | 743394 | 0.00246 | 0.70 |
| HIVEP1 | 758037 | 0.00248 | 0.79 |
| ATP5G3 | 740090 | 0.00255 | 0.82 |
| UBE2D2 | 594655 | 0.00275 | 0.81 |
| MEOX1 | 760065 | 0.00296 | 0.75 |
| FOXN4 | 731076 | 0.00297 | 0.78 |
| SPDEF | 1188588 | 0.00302 | 0.77 |
| LOC56251 | 137478 | 0.00312 | 0.77 |
| LPL | 868169 | 0.0032 | 0.72 |
| BAP1 | 1012990 | 0.00336 | 0.73 |
| SIAT8C | 382069 | 0.00336 | 0.77 |
| TOP1 | 666425 | 0.00339 | 0.85 |
| IMAGE:135203 | 135203 | 0.00347 | 0.79 |
| Transcribed | | | |
| locus | | | |
| CASP5 | 341763 | 0.00364 | 0.67 |
| EPRS | 669549 | 0.00368 | 0.83 |
| FAM44C | 726894 | 0.0038 | 0.84 |
| HMG20A | 731277 | 0.00417 | 0.72 |
| IMAGE:485104 | 485104 | 0.00423 | 0.69 |
| Transcribed | | | |
| locus | | | |
| SCEL | 668239 | 0.00425 | 0.82 |
| IRF4 | 682207 | 0.00432 | 0.74 |
| RIN1 | 741406 | 0.0044 | 0.78 |
| PLCG2 | 201467 | 0.00461 | 0.78 |
| FABP5 | 1088781 | 0.00488 | 0.47 |
| MAK | 382002 | 0.00498 | 0.74 |
| ELK4 | 236155 | 0.00507 | 0.76 |
| IMAGE:731758 | 731758 | 0.00521 | 0.77 |
| Transcribed | | | |
| locus, | | | |
| weakly | | | |
| similar to | | | |
| NP_081070.1 | | | |
| kiaa-iso | | | |
| protein | | | |
| homolog; | | | |
| Band | | | |
| 47B [Mus | | | |
| musculus] | | | |
| DAPK1 | 364934 | 0.00547 | 0.82 |
| MRC2 | 235882 | 0.00557 | 0.58 |
| SYNPO | 178792 | 0.00562 | 0.81 |
| KIAA1754L | 365056 | 0.00563 | 0.76 |
| RGS20 | 668152 | 0.00594 | 0.78 |
| HCLS1 | 665452 | 0.00599 | 0.70 |
| NY-REN- | 136646 | 0.00606 | 0.79 |
| 41 | | | |
| CENTD1 | 666110 | 0.0061 | 0.81 |
| PHC2 | 668146 | 0.00637 | 0.79 |
| MAP2 | 347503 | 0.00639 | 0.74 |
| IMAGE:744616 | 744616 | 0.00648 | 0.78 |
| DNA | | | |
| damage | | | |
| repair | | | |
| and | | | |
| recombination | | | |
| protein | | | |
| RAD52 | | | |
| pseudogene | | | |
| IMAGE:177857 | 177857 | 0.00656 | 0.79 |
| Transcribed | | | |
| locus | | | |
| IL10RA | 258747 | 0.00664 | 0.70 |
| WDR20 | 665156 | 0.00676 | 0.81 |
| CTRL | 1308954 | 0.00707 | 0.82 |
| FBXO46 | 471664 | 0.00716 | 0.72 |
| CHRDL2 | 485872 | 0.0075 | 0.80 |
| LRPAP1 | 587186 | 0.00762 | 0.60 |
| CCL28 | 136919 | 0.00767 | 0.84 |
| SLC39A2 | 504596 | 0.00779 | 0.83 |
| CITED1 | 265558 | 0.0078 | 0.49 |
| LARS | 135407 | 0.00805 | 0.79 |
| ICAM3 | 156183 | 0.00823 | 0.79 |
| IMAGE:484577 | 484577 | 0.00852 | 0.68 |
| Transcribed | | | |
| locus, | | | |
| weakly | | | |
| similar to | | | |
| NP_000991.1 | | | |
| ribosomal | | | |
| protein | | | |
| L39; 60S | | | |
| ribosomal | | | |
| protein | | | |
| L39 | | | |
| [Homo | | | |
| sapiens] | | | |
| RASGRP1 | 725707 | 0.00864 | 0.68 |
| C6orf176 | 173200 | 0.00866 | 0.83 |
| FLJ22794 | 137454 | 0.00895 | 0.82 |
| IMAGE:743078 | 743078 | 0.00904 | 0.83 |
| IGKV1-5 | 155345 | 0.00922 | 0.83 |
| IMAGE:666794 | 666794 | 0.00944 | 0.74 |
| Hypothetical | | | |
| LOC388170 | | | |
| PPP2CB | 667375 | 0.00968 | 0.84 |
| HT007 | 135303 | 0.00976 | 0.84 |
| LOC56930 | 134976 | 0.00977 | 0.86 |
| ECM1 | 301122 | 0.0098 | 0.30 |
| LCN2 | 741497 | 0.01015 | 0.35 |
| PITPNA | 725585 | 0.01039 | 0.70 |
| IMAGE:743579 | 743579 | 0.01044 | 0.74 |
| RUNX1 | 263251 | 0.01059 | 0.79 |
| IMAGE:744074 | 744074 | 0.01063 | 0.75 |
| Transcribed | | | |
| locus | | | |
| SAFB | 42280 | 0.01086 | 0.69 |
| C9orf10OS | 742695 | 0.01194 | 0.84 |
| MGC23909 | 731598 | 0.012 | 0.77 |
| CLNS1A | 666426 | 0.01248 | 0.82 |
| IMAGE:743603 | 743603 | 0.01255 | 0.78 |
| IMAGE:665403 | 665403 | 0.01258 | 0.83 |
| Transcribed | | | |
| locus | | | |
| KLF4 | 188232 | 0.01265 | 0.63 |
| IMAGE:1859532 | 1859532 | 0.01275 | 0.83 |
| RFPL1 | 290265 | 0.01296 | 0.81 |
| HMGB2 | 884365 | 0.01308 | 0.84 |
| COX6A1 | 512910 | 0.01322 | 0.84 |
| ACADSB | 687953 | 0.01324 | 0.77 |
| IMAGE:731357 | 731357 | 0.01343 | 0.87 |
| Transcribed | | | |
| locus | | | |
| SAP30L | 723950 | 0.0137 | 0.84 |
| PSG3 | 136747 | 0.0142 | 0.79 |
| PCSK6 | 31924 | 0.01471 | 0.71 |
| CASP7 | 279470 | 0.01475 | 0.86 |
| AFTIPHILIN | 741790 | 0.01511 | 0.79 |
| GATA4 | 781738 | 0.01514 | 0.80 |
| FXYD1 | 204686 | 0.01535 | 0.83 |
| ATP11B | 666334 | 0.01546 | 0.73 |
| S100A10 | 119939 | 0.01567 | 0.65 |
| COX5A | 1085884 | 0.01572 | 0.80 |
| CERKL | 489326 | 0.01615 | 0.74 |
| CD5 | 356841 | 0.01627 | 0.75 |
| NME7 | 743982 | 0.01628 | 0.76 |
| CCL19 | 1707527 | 0.01688 | 0.32 |
| JUP | 35628 | 0.01697 | 0.70 |
| C21orf84 | 730814 | 0.01699 | 0.77 |
| HOXB6 | 738358 | 0.01764 | 0.84 |
| FLJ25067 | 667252 | 0.01864 | 0.69 |
| IMAGE:740927 | 740927 | 0.01893 | 0.76 |
| MRC2 | 342581 | 0.01961 | 0.72 |
| GPC3 | 137131 | 0.01963 | 0.82 |
| CCL22 | 2150502 | 0.02001 | 0.85 |
| LILRA1 | 2032639 | 0.02014 | 0.82 |
| STAT2 | 2306096 | 0.02037 | 0.85 |
| HRK | 767779 | 0.02045 | 0.69 |
| PDXP | 743182 | 0.02046 | 0.84 |
| ALDH1A1 | 309697 | 0.02059 | 0.78 |
| DKFZP564D166 | 744374 | 0.02064 | 0.81 |
| PIK3CA | 2238108 | 0.02077 | 0.54 |
| IMAGE:136223 | 136223 | 0.02096 | 0.85 |
| IMAGE:666279 | 666279 | 0.02123 | 0.72 |
| CDNA | | | |
| FLJ30779 | | | |
| fis, | | | |
| clone | | | |
| FEBRA2000815 | | | |
| MFAP3L | 726821 | 0.02166 | 0.81 |
| PSMD11 | 383945 | 0.02171 | 0.77 |
| HTATIP2 | 726618 | 0.02245 | 0.83 |
| IMAGE:23817 | 23817 | 0.02275 | 0.80 |
| CDNA | | | |
| FLJ12202 | | | |
| fis, | | | |
| clone | | | |
| MAMMA1000908 | | | |
| POU2F2 | 188393 | 0.02319 | 0.72 |
| GPI | 741474 | 0.02319 | 0.80 |
| FGF1 | 25895 | 0.0233 | 0.82 |
| EGFR | 135980 | 0.02368 | 0.80 |
| IMAGE:136014 | 136014 | 0.02388 | 0.80 |
| ZNF250 | 741066 | 0.02392 | 0.53 |
| IMAGE:382521 | 382521 | 0.02438 | 0.70 |
| LOC286076 | 731275 | 0.02463 | 0.83 |
| IMAGE:136598 | 136598 | 0.02485 | 0.88 |
| MRNA; | | | |
| cDNA | | | |
| DKFZp686D22106 | | | |
| (from | | | |
| clone | | | |
| DKFZp686D22106) | | | |
| CCR7 | 2345206 | 0.02512 | 0.78 |
| GNAS | 382791 | 0.02579 | 0.70 |
| ME3 | 724238 | 0.02586 | 0.74 |
| RGS19 | 485803 | 0.02627 | 0.85 |
| BCAP29 | 725970 | 0.02682 | 0.79 |
| HOXD11 | 682119 | 0.02693 | 0.80 |
| CD69 | 276727 | 0.02719 | 0.82 |
| FLJ37587 | 668476 | 0.02773 | 0.87 |
| TM4SF8 | 713647 | 0.02822 | 0.83 |
| IMAGE:742061 | 742061 | 0.02872 | 0.78 |
| TFF1 | 1075949 | 0.02928 | 0.74 |
| CASP10 | 241481 | 0.02963 | 0.75 |
| CXCL13 | 347362 | 0.03057 | 0.87 |
| HLA- | 725548 | 0.03155 | 0.74 |
| DPB1 | | | |
| IL13RA1 | 1492440 | 0.03217 | 0.88 |
| IMAGE:1117183 | 1117183 | 0.03277 | 0.79 |
| NME1 | 726600 | 0.03337 | 0.82 |
| CKLFSF3 | 489249 | 0.03492 | 0.88 |
| CCR7 | 2345206 | 0.03508 | 0.86 |
| CCND3 | 327182 | 0.03567 | 0.85 |
| CAV1 | 133531 | 0.03597 | 0.80 |
| PTHLH | 2214396 | 0.03608 | 0.74 |
| LILRB5 | 71428 | 0.03622 | 0.75 |
| IMAGE:665342 | 665342 | 0.03709 | 0.90 |
| Transcribed | | | |
| locus, | | | |
| strongly | | | |
| similar to | | | |
| NP_065847.1 | | | |
| semaphorin | | | |
| 6A1; | | | |
| semaphorin | | | |
| 6A-1 | | | |
| [Homo | | | |
| sapiens] | | | |
| LOC253981 | 742743 | 0.0371 | 0.82 |
| CUGBP2 | 666235 | 0.03791 | 0.88 |
| TPD52 | 743259 | 0.03834 | 0.46 |
| JAK1 | 2030501 | 0.03845 | 0.80 |
| OAS1 | 666703 | 0.0394 | 0.74 |
| KCNQ2 | 179534 | 0.03951 | 0.83 |
| ZNF395 | 744983 | 0.04028 | 0.85 |
| CASP4 | 356960 | 0.04144 | 0.80 |
| TGFB3 | 1561035 | 0.04297 | 0.84 |
| IMAGE:726647 | 726647 | 0.04331 | 0.83 |
| Transcribed | | | |
| locus | | | |
| MAPK14 | 1741589 | 0.04332 | 0.89 |
| MAN1A1 | 137719 | 0.04366 | 0.68 |
| NTRK2 | 2048801 | 0.04379 | 0.78 |
| WIPI49 | 487148 | 0.04534 | 0.88 |
| SEMA3A | 33664 | 0.046 | 0.83 |
| PTK2B | 43541 | 0.0464 | 0.88 |
| ULK4 | 744895 | 0.04646 | 0.85 |
| IMAGE:687667 | 687667 | 0.04704 | 0.58 |
| CDNA | | | |
| clone | | | |
| IMAGE:4811759, | | | |
| partial | | | |
| cds | | | |
| AMD1 | 132752 | 0.04762 | 0.90 |
| RUNX3 | 122874 | 0.04914 | 0.80 |
| LILRA1 | 2032639 | 0.04959 | 0.85 |

TABLE 8

| | FTA-a | Over | |
|---|---|---|---|
| name | cloid | p-value | fold |
| ZNF76 | 745003 | 3.83E−05 | 1.61 |
| CABIN1 | 1844968 | 0.00023 | 1.64 |
| PRUNE | 364324 | 2.36E−04 | 1.36 |
| CST3 | 357201 | 5.87E−04 | 1.26 |
| BAT3 | 24392 | 0.00067 | 1.36 |
| PLXNB2 | 1420676 | 6.94E−04 | 1.80 |
| FBXW8 | 687532 | 7.31E−04 | 1.21 |
| PPARD | 724562 | 0.00077 | 1.27 |
| LOC147650 | 729510 | 8.26E−04 | 1.30 |
| DAP | 725371 | 9.98E−04 | 1.55 |
| EIF4G1 | 24083 | 1.02E−03 | 1.64 |
| KIAA1196 | 738938 | 1.09E−03 | 1.62 |
| PH-4 | 730938 | 1.39E−03 | 1.43 |
| GATA3 | 148796 | 1.42E−03 | 1.43 |
| CORO1B | 137205 | 1.47E−03 | 1.37 |
| OTUB1 | 174683 | 1.49E−03 | 1.37 |
| IMAGE:21808 | 21808 | 1.94E−03 | 1.34 |
| Transcribed | | | |
| locus | | | |
| S100A2 | 3659591 | 2.01E−03 | 1.32 |
| KRTHA4 | 325155 | 2.19E−03 | 1.60 |
| ARHGAP21 | 666792 | 2.57E−03 | 1.19 |
| FCGBP | 154172 | 2.63E−03 | 1.35 |
| RAD51L1 | 295412 | 2.70E−03 | 1.57 |
| IMAGE:176543 | 176543 | 2.87E−03 | 1.31 |
| FLJ14981 | 24532 | 3.45E−03 | 1.41 |
| COL18A1 | 359202 | 0.00366 | 1.64 |
| CCNE1 | 68950 | 4.27E−03 | 1.43 |
| SGCG | 2046361 | 4.36E−03 | 1.22 |
| PVRL2 | 725364 | 4.46E−03 | 1.27 |
| IMAGE:741178 | 741178 | 4.49E−03 | 1.30 |
| CDNA | | | |
| FLJ38849 fis, | | | |
| clone | | | |
| MESAN2008936 | | | |
| RBM25 | 668360 | 4.64E−03 | 1.35 |
| BCL2L11 | 300194 | 4.68E−03 | 1.56 |
| IMAGE:23872 | 23872 | 4.96E−03 | 1.17 |
| AF034176 | | | |
| Human mRNA | | | |
| (Tripodis and | | | |
| Ragoussis) | | | |
| Homo sapiens | | | |
| cDNA clone | | | |
| ntcon5 contig | | | |
| FLJ13491 | 364352 | 0.00525 | 1.35 |
| NDUFA7 | 364469 | 0.00528 | 1.24 |
| LOC146909 | 726353 | 0.00531 | 1.32 |
| LGALS3BP | 742100 | 5.46E−03 | 1.41 |
| FLJ12895 | 23271 | 5.96E−03 | 1.33 |
| C9orf119 | 667112 | 0.00607 | 1.26 |
| GPR108 | 25949 | 6.22E−03 | 1.42 |
| RPS6KA2 | 22711 | 6.63E−03 | 1.67 |
| IMAGE:744055 | 744055 | 7.01E−03 | 1.57 |
| Transcribed | | | |
| locus | | | |
| RTN3 | 430263 | 7.06E−03 | 1.22 |
| HDAC9 | 738506 | 7.91E−03 | 1.22 |
| IMAGE:738945 | 738945 | 7.97E−03 | 1.23 |
| GGN | 743245 | 8.24E−03 | 1.21 |
| TM4SF9 | 812967 | 8.54E−03 | 1.40 |
| PLA2G6 | 744087 | 8.74E−03 | 1.40 |
| UBE1L | 250883 | 9.66E−03 | 1.37 |
| SLC25A23 | 741954 | 9.88E−03 | 1.49 |
| DCI | 667892 | 0.00991 | 1.28 |
| PP1201 | 364974 | 0.00997 | 1.20 |
| IMAGE:744505 | 744505 | 0.01063 | 1.44 |
| TLN1 | 187482 | 1.11E−02 | 1.38 |
| LOC51760 | 52226 | 1.15E−02 | 1.42 |
| DJ159A19.3 | 23945 | 1.15E−02 | 1.47 |
| TPCN1 | 179288 | 1.28E−02 | 1.29 |
| AGPAT3 | 738507 | 1.29E−02 | 1.18 |
| GCLC | 1526058 | 1.38E−02 | 1.20 |
| DNALI1 | 782688 | 1.43E−02 | 1.44 |
| IMAGE:174861 | 174861 | 1.53E−02 | 1.31 |
| Transcribed | | | |
| locus | | | |
| HDAC6 | 669295 | 1.57E−02 | 1.18 |
| C7orf27 | 713859 | 1.63E−02 | 1.23 |
| ACTR1B | 136868 | 1.73E−02 | 1.24 |
| GNB1 | 24300 | 1.74E−02 | 1.16 |
| IMAGE:731726 | 731726 | 1.80E−02 | 1.42 |
| Transcribed | | | |
| locus | | | |
| RANBP10 | 682251 | 1.83E−02 | 1.21 |
| PTDSS2 | 21716 | 1.83E−02 | 1.19 |
| PIAS1 | 38789 | 1.85E−02 | 1.20 |
| ASGR1 | 25883 | 1.87E−02 | 1.91 |
| PDAP1 | 172693 | 1.88E−02 | 1.20 |
| TETRAN | 725340 | 1.96E−02 | 1.36 |
| AMPD2 | 669141 | 2.00E−02 | 1.23 |
| BAD | 712295 | 2.09E−02 | 1.21 |
| FOXM1 | 23641 | 2.22E−02 | 1.19 |
| PDCD1LG2 | 738944 | 2.27E−02 | 1.21 |
| IMAGE:1716286 | 1716286 | 2.27E−02 | 1.44 |
| PNKP | 723867 | 2.27E−02 | 1.21 |
| SSH3 | 726272 | 2.46E−02 | 1.17 |
| HSPB1 | 23827 | 2.54E−02 | 1.37 |
| CCNE1 | 357807 | 2.67E−02 | 1.24 |
| IMAGE:136801 | 136801 | 2.67E−02 | 1.19 |
| AXIN2 | 135887 | 0.02753 | 1.16 |
| IMAGE:2254555 | 2254555 | 2.77E−02 | 1.20 |
| KCNC3 | 53333 | 2.81E−02 | 1.42 |
| IMAGE:472111 | 472111 | 2.83E−02 | 1.49 |
| Transcribed | | | |
| locus | | | |
| CDH1 | 214008 | 2.94E−02 | 1.24 |
| KIAA1838 | 744106 | 2.96E−02 | 1.15 |
| ANAPC2 | 136462 | 2.97E−02 | 1.20 |
| MGC5178 | 725533 | 0.03005 | 1.20 |
| PBXIP1 | 366042 | 3.08E−02 | 1.25 |
| CaMKIINalpha | 173820 | 3.09E−02 | 1.33 |
| MBNL1 | 136114 | 3.38E−02 | 1.30 |
| FLJ10707 | 364575 | 3.41E−02 | 1.15 |
| GALT | 68972 | 3.47E−02 | 1.19 |
| LPXN | 687679 | 3.47E−02 | 1.16 |
| SENP6 | 366436 | 3.51E−02 | 1.18 |
| EPHA4 | 2237263 | 3.56E−02 | 1.37 |
| SLA | 32339 | 3.61E−02 | 1.41 |
| IMAGE:26486 | 26486 | 3.67E−02 | 1.23 |
| CDNA | | | |
| FLJ43345 fis, | | | |
| clone | | | |
| NT2RI3008228 | | | |
| NF2 | 1698236 | 3.78E−02 | 1.44 |
| CRLF3 | 279150 | 3.84E−02 | 1.17 |
| IL11RA | 1101773 | 3.84E−02 | 1.33 |
| FLJ30707 | 666986 | 3.89E−02 | 1.16 |
| IMAGE:669470 | 669470 | 4.03E−02 | 1.18 |
| CDNA clone | | | |
| IMAGE:5206119, | | | |
| partial cds | | | |
| PPP1CA | 257259 | 4.05E−02 | 1.16 |
| RIPK3 | 667313 | 4.06E−02 | 1.31 |
| OAZ2 | 128694 | 4.33E−02 | 1.15 |
| GALNT9 | 178750 | 4.35E−02 | 1.31 |
| LILRB4 | 2341829 | 4.45E−02 | 1.15 |
| NIFIE14 | 472160 | 4.53E−02 | 1.16 |
| IMAGE:727123 | 727123 | 4.64E−02 | 1.14 |
| Transcribed | | | |
| locus, strongly | | | |
| similar to | | | |
| NP_004491.1 | | | |
| heterogeneous | | | |
| nuclear | | | |
| ribonucleoprotein | | | |
| C isoform b; | | | |
| nuclear | | | |
| ribonucleoprotein | | | |
| particle C2 | | | |
| protein; nuclear | | | |
| ribonucleoprotein | | | |
| particle C1 | | | |
| protein [Homo | | | |
| sapiens] | | | |

TABLE 9

| | | FTA- | |
|---|---|---|---|
| | | atypical | Under |
| name | cloid | p-value | fold |
| NBL1 | 503874 | 5.13E−11 | 0.22 |
| DF | 666128 | 2.64E−09 | 0.34 |
| APOD | 838611 | 2.03E−07 | 0.19 |
| KLF1 | 208991 | 4.17E−07 | 0.37 |
| MGC99813 | 739097 | 6.06E−07 | 0.3 |
| IMAGE:1089025 | 1089025 | 6.77E−07 | 0.25 |
| HLA-DPB1 | 725548 | 1.15E−06 | 0.5 |
| CRABP1 | 739193 | 1.71E−06 | 0.27 |
| MMP2 | 155839 | 4.59E−06 | 0.52 |
| MATN2 | 28584 | 5.28E−06 | 0.45 |
| LILRB2 | 202897 | 7.11E−06 | 0.52 |
| SIGLEC7 | 743331 | 7.86E−06 | 0.64 |
| IMAGE:743619 | 743619 | 9.02E−06 | 0.61 |
| NEK6 | 725345 | 9.81E−06 | 0.6 |
| CXCL14 | 345034 | 1.16E−05 | 0.2 |
| BAK1 | 235938 | 1.44E−05 | 0.65 |
| CPXM2 | 729924 | 1.73E−05 | 0.56 |
| ETHE1 | 681957 | 1.95E−05 | 0.73 |
| LRP1 | 30219 | 1.98E−05 | 0.55 |
| GPNMB | 773330 | 2.86E−05 | 0.25 |
| APOL1 | 665632 | 3.32E−05 | 0.4 |
| NTRK2 | 2048801 | 3.48E−05 | 0.49 |
| MEOX1 | 760065 | 3.88E−05 | 0.6 |
| S100A10 | 119939 | 3.89E−05 | 0.59 |
| BAI3 | 50491 | 3.90E−05 | 0.58 |
| GC | 195340 | 4.63E−05 | 0.61 |
| C10orf116 | 740941 | 5.60E−05 | 0.5 |
| PLCG2 | 201467 | 6.66E−05 | 0.65 |
| VAV2 | 1581686 | 6.79E−05 | 0.69 |
| IMAGE:743603 | 743603 | 7.55E−05 | 0.66 |
| SLC17A2 | 207920 | 9.06E−05 | 0.43 |
| IMAGE:731616 | 731616 | 1.16E−04 | 0.46 |
| MRNA; | | | |
| cDNA | | | |
| DKFZp686A102 | | | |
| (from | | | |
| clone | | | |
| DKFZp686A102) | | | |
| LILRB5 | 71428 | 1.50E−04 | 0.64 |
| SYP | 30471 | 1.78E−04 | 0.65 |
| PTGES | 504646 | 1.83E−04 | 0.77 |
| CCDC6 | 487848 | 1.97E−04 | 0.67 |
| MRC2 | 342581 | 2.34E−04 | 0.51 |
| PDCD1LG1 | 1942634 | 2.77E−04 | 0.72 |
| PIK3CA | 2238108 | 2.98E−04 | 0.37 |
| DUSP24 | 740158 | 3.21E−04 | 0.4 |
| SAFB | 42280 | 3.29E−04 | 0.53 |
| GJA5 | 196338 | 3.41E−04 | 0.45 |
| IMAGE:742739 | 742739 | 3.99E−04 | 0.57 |
| CDNA | | | |
| FLJ12931 fis, | | | |
| clone | | | |
| NT2RP2004861 | | | |
| IMAGE:743615 | 743615 | 4.35E−04 | 0.72 |
| Full- | | | |
| length cDNA | | | |
| clone | | | |
| CS0DK001YE19 | | | |
| of HeLa | | | |
| cells Cot 25- | | | |
| normalized of | | | |
| Homo | | | |
| sapiens | | | |
| (human) | | | |
| BIK | 1074708 | 4.37E−04 | 0.75 |
| GTPBP1 | 366484 | 4.94E−04 | 0.69 |
| GCK | 30981 | 5.91E−04 | 0.73 |
| FLJ27523 | 743246 | 6.04E−04 | 0.43 |
| LASP1 | 592598 | 6.09E−04 | 0.47 |
| IMAGE:485104 | 485104 | 6.19E−04 | 0.64 |
| Transcribed | | | |
| locus | | | |
| STX7 | 174396 | 6.31E−04 | 0.54 |
| PPIL4 | 364777 | 6.96E−04 | 0.4 |
| C19orf33 | 379540 | 7.02E−04 | 0.58 |
| IL2RA | 3054031 | 7.14E−04 | 0.69 |
| IFNAR2 | 123950 | 7.17E−04 | 0.59 |
| CD3E | 1536968 | 7.38E−04 | 0.61 |
| MATN2 | 366100 | 7.66E−04 | 0.64 |
| CERKL | 489326 | 7.80E−04 | 0.64 |
| IMAGE:24587 | 24587 | 7.81E−04 | 0.6 |
| DEFB1 | 665086 | 7.91E−04 | 0.36 |
| IMAGE:342256 | 342256 | 8.29E−04 | 0.28 |
| SEPT-05 | 448163 | 9.20E−04 | 0.28 |
| JUP | 35628 | 1.01E−03 | 0.56 |
| USP4 | 544818 | 1.06E−03 | 0.64 |
| KCNQ2 | 179534 | 1.10E−03 | 0.62 |
| WDR20 | 665156 | 1.15E−03 | 0.78 |
| CAV1 | 309645 | 1.18E−03 | 0.5 |
| SALL4 | 726454 | 1.18E−03 | 0.48 |
| IMAGE:738896 | 738896 | 1.20E−03 | 0.32 |
| PCSK6 | 31924 | 1.27E−03 | 0.63 |
| HCLS1 | 665452 | 1.27E−03 | 0.66 |
| DLX5 | 564878 | 1.27E−03 | 0.55 |
| TRAF5 | 1286238 | 1.48E−03 | 0.76 |
| COL27A1 | 379517 | 1.59E−03 | 0.66 |
| IMAGE:177857 | 177857 | 1.59E−03 | 0.63 |
| Transcribed | | | |
| locus | | | |
| IMAGE:744410 | 744410 | 1.61E−03 | 0.71 |
| Transcribed | | | |
| locus | | | |
| OBSCN | 730926 | 1.67E−03 | 0.73 |
| E2F5 | 809828 | 1.71E−03 | 0.56 |
| SLAMF8 | 288807 | 1.84E−03 | 0.74 |
| IMAGE:740105 | 740105 | 2.04E−03 | 0.67 |
| NCAM1 | 366842 | 2.06E−03 | 0.72 |
| FRZB | 146049 | 2.23E−03 | 0.62 |
| AQP11 | 713831 | 2.30E−03 | 0.6 |
| COL1A1 | 140347 | 2.30E−03 | 0.6 |
| IMAGE:666279 | 666279 | 2.46E−03 | 0.62 |
| CDNA | | | |
| FLJ30779 fis, | | | |
| clone | | | |
| FEBRA2000815 | | | |
| CASP5 | 341763 | 2.47E−03 | 0.53 |
| CFH | 665784 | 2.51E−03 | 0.53 |
| FLJ14981 | 24532 | 2.52E−03 | 0.65 |
| SIAT8B | 33133 | 2.52E−03 | 0.68 |
| HSPC159 | 365045 | 2.67E−03 | 0.74 |
| BAK1 | 1288183 | 2.71E−03 | 0.58 |
| CAV1 | 133531 | 2.80E−03 | 0.59 |
| IMAGE:744926 | 744926 | 2.95E−03 | 0.61 |
| Transcribed | | | |
| locus | | | |
| FCHO1 | 742763 | 3.01E−03 | 0.78 |
| IMAGE:472111 | 472111 | 3.05E−03 | 0.59 |
| Transcribed | | | |
| locus | | | |
| BAP1 | 1012990 | 3.05E−03 | 0.66 |
| TPD52 | 743259 | 3.11E−03 | 0.29 |
| CCL19 | 1707527 | 3.14E−03 | 0.21 |
| EMX2 | 365121 | 3.16E−03 | 0.69 |
| BIC | 743270 | 3.26E−03 | 0.65 |
| IMAGE:666315 | 666315 | 3.28E−03 | 0.66 |
| Homo | | | |
| sapiens, | | | |
| clone | | | |
| IMAGE:5312086, | | | |
| mRNA | | | |
| IMAGE:2484270 | 2484270 | 3.50E−03 | 0.67 |
| KLHL9 | 471696 | 3.57E−03 | 0.66 |
| APIN | 364706 | 3.70E−03 | 0.61 |
| IMAGE:364741 | 364741 | 4.03E−03 | 0.62 |
| VAV1 | 80384 | 4.05E−03 | 0.53 |
| MGP | 590264 | 4.05E−03 | 0.66 |
| SFRP4 | 285693 | 4.12E−03 | 0.54 |
| NOLA2 | 173296 | 4.30E−03 | 0.8 |
| TGFB3 | 1561035 | 4.48E−03 | 0.66 |
| HBB | 469549 | 4.48E−03 | 0.44 |
| LILRB3 | 1422194 | 4.59E−03 | 0.62 |
| IMAGE:731689 | 731689 | 4.76E−03 | 0.73 |
| Transcribed | | | |
| locus | | | |
| IMAGE:667527 | 667527 | 4.81E−03 | 0.62 |
| TLX3 | 1860115 | 4.86E−03 | 0.63 |
| ID1 | 1087348 | 4.93E−03 | 0.6 |
| FXYD1 | 204686 | 4.96E−03 | 0.65 |
| TFF1 | 1075949 | 5.04E−03 | 0.58 |
| SLPI | 378813 | 5.42E−03 | 0.56 |
| SYT1 | 28511 | 5.47E−03 | 0.73 |
| RNF32 | 731422 | 5.77E−03 | 0.54 |
| TOSO | 813174 | 5.84E−03 | 0.55 |
| IFI30 | 740931 | 5.91E−03 | 0.6 |
| ASGR1 | 25883 | 6.02E−03 | 0.64 |
| F10 | 310519 | 6.20E−03 | 0.73 |
| LEFTY2 | 340657 | 6.42E−03 | 0.69 |
| IMAGE:727289 | 727289 | 6.73E−03 | 0.65 |
| Transcribed | | | |
| locus | | | |
| IMAGE:743722 | 743722 | 6.88E−03 | 0.7 |
| NCB5OR | 743367 | 6.92E−03 | 0.55 |
| SERPINB5 | 1662274 | 7.01E−03 | 0.72 |
| AXL | 364083 | 7.01E−03 | 0.59 |
| CREB3L2 | 136399 | 7.23E−03 | 0.56 |
| GRIK2 | 731363 | 7.95E−03 | 0.74 |
| IMAGE:743579 | 743579 | 7.97E−03 | 0.55 |
| FABP5 | 1088781 | 8.38E−03 | 0.45 |
| FOLR1 | 131839 | 8.85E−03 | 0.65 |
| GJA1 | 839101 | 8.97E−03 | 0.63 |
| CCL13 | 2449149 | 8.99E−03 | 0.77 |
| BMP5 | 1846326 | 9.07E−03 | 0.72 |
| RIN1 | 741406 | 9.10E−03 | 0.67 |
| SIAT7B | 823590 | 9.35E−03 | 0.66 |
| SCMH1 | 724014 | 1.00E−02 | 0.6 |
| GALNT9 | 178750 | 1.01E−02 | 0.69 |
| IFITM3 | 713623 | 1.03E−02 | 0.73 |
| HRK | 767779 | 1.05E−02 | 0.69 |
| CDR2 | 366067 | 1.08E−02 | 0.83 |
| IMAGE:743078 | 743078 | 1.08E−02 | 0.82 |
| IMAGE:383966 | 383966 | 1.09E−02 | 0.67 |
| RAD51L1 | 295412 | 1.09E−02 | 0.68 |
| MAPK3 | 323438 | 1.10E−02 | 0.68 |
| ZNF319 | 364729 | 1.14E−02 | 0.45 |
| RUNX3 | 122874 | 1.17E−02 | 0.73 |
| PARD3 | 724642 | 1.22E−02 | 0.73 |
| SOD3 | 795309 | 1.23E−02 | 0.52 |
| RPL27A | 178255 | 1.28E−02 | 0.58 |
| BTG1 | 382760 | 1.29E−02 | 0.8 |
| SEMA7A | 135941 | 1.30E−02 | 0.55 |
| KLK2 | 1102600 | 1.31E−02 | 0.65 |
| BMP4 | 69166 | 1.32E−02 | 0.72 |
| UNC13D | 231903 | 1.41E−02 | 0.55 |
| IGSF10 | 682276 | 1.44E−02 | 0.8 |
| IMAGE:1716286 | 1716286 | 1.48E−02 | 0.63 |
| HAS3 | 667533 | 1.51E−02 | 0.69 |
| SDHA | 40304 | 1.52E−02 | 0.75 |
| FLJ22386 | 32917 | 1.53E−02 | 0.79 |
| FLJ23577 | 668452 | 1.60E−02 | 0.61 |
| MGAT3 | 731060 | 1.62E−02 | 0.79 |
| TRIP12 | 665820 | 1.62E−02 | 0.62 |
| MATN1 | 1624260 | 1.67E−02 | 0.78 |
| CD38 | 1352408 | 1.82E−02 | 0.74 |
| FBXO46 | 471664 | 1.84E−02 | 0.68 |
| ZNF251 | 486401 | 1.86E−02 | 0.56 |
| LOC388284 | 731722 | 1.88E−02 | 0.75 |
| PDLIM1 | 135689 | 1.91E−02 | 0.7 |
| ISG20 | 740604 | 1.92E−02 | 0.69 |
| BRAF | 2139164 | 2.05E−02 | 0.82 |
| AP2S1 | 739109 | 2.16E−02 | 0.87 |
| ITGA5 | 135671 | 2.31E−02 | 0.74 |
| OAS1 | 666703 | 2.32E−02 | 0.63 |
| F5 | 433155 | 2.33E−02 | 0.7 |
| THAP3 | 687800 | 2.37E−02 | 0.56 |
| TREM2 | 502244 | 2.41E−02 | 0.78 |
| TNFRSF1B | 73703 | 2.43E−02 | 0.82 |
| IMAGE:484577 | 484577 | 2.43E−02 | 0.69 |
| Transcribed | | | |
| locus, weakly | | | |
| similar to | | | |
| NP_000991.1 | | | |
| ribosomal | | | |
| protein L39; | | | |
| 60S | | | |
| ribosomal | | | |
| protein L39 | | | |
| [Homo | | | |
| sapiens] | | | |
| ATR | 1251197 | 2.46E−02 | 0.83 |
| MUTED | 731202 | 2.47E−02 | 0.65 |
| TM4SF9 | 381032 | 2.49E−02 | 0.76 |
| BST2 | 811024 | 2.50E−02 | 0.75 |
| MAP2 | 347503 | 2.50E−02 | 0.71 |
| MGC5395 | 238840 | 2.53E−02 | 0.79 |
| BID | 128065 | 2.60E−02 | 0.82 |
| GNAS | 382791 | 2.65E−02 | 0.66 |
| APOE | 1870594 | 2.66E−02 | 0.69 |
| SPP1 | 258114 | 2.66E−02 | 0.52 |
| C6orf216 | 731742 | 2.70E−02 | 0.8 |
| SYN3 | 727056 | 2.72E−02 | 0.66 |
| GATA4 | 781738 | 2.92E−02 | 0.81 |
| LRRIQ2 | 487152 | 3.01E−02 | 0.75 |
| MCC | 731305 | 3.16E−02 | 0.59 |
| IMAGE:731726 | 731726 | 3.23E−02 | 0.73 |
| Transcribed | | | |
| locus | | | |
| BCL2 | 342181 | 3.33E−02 | 0.82 |
| IMAGE:745512 | 745512 | 3.38E−02 | 0.85 |
| CASP4 | 356960 | 3.45E−02 | 0.75 |
| GLT8D2 | 365271 | 3.51E−02 | 0.64 |
| MT1F | 78353 | 3.57E−02 | 0.64 |
| IMAGE:730924 | 730924 | 3.74E−02 | 0.74 |
| Transcribed | | | |
| locus | | | |
| IMAGE:742853 | 742853 | 4.03E−02 | 0.67 |
| KLRB1 | 2091591 | 4.11E−02 | 0.73 |
| WDR5 | 731023 | 4.14E−02 | 0.8 |
| C6orf176 | 173200 | 4.19E−02 | 0.9 |
| SLC39A2 | 504596 | 4.25E−02 | 0.75 |
| ARHGAP21 | 666792 | 4.27E−02 | 0.86 |
| IMAGE:383718 | 383718 | 4.31E−02 | 0.77 |
| MAK | 382002 | 4.33E−02 | 0.83 |
| GPI | 741474 | 4.37E−02 | 0.78 |
| C20orf19 | 366032 | 4.50E−02 | 0.73 |
| LOC285500 | 135766 | 4.56E−02 | 0.68 |
| PSMD11 | 383945 | 4.57E−02 | 0.77 |
| MUC1 | 840687 | 4.79E−02 | 0.71 |
| PITPNC1 | 364436 | 4.79E−02 | 0.74 |
| MKKS | 729957 | 4.82E−02 | 0.79 |
| KLHL9 | 501527 | 4.82E−02 | 0.83 |
| OSR1 | 364686 | 4.96E−02 | 0.75 |

TABLE 10

| | FTA- | | |
|---|---|---|---|
| | atypical | Over | |
| name | cloid | p-value | fold |
| SNCB | 50202 | 6.55E−04 | 2.00 |
| DJ462O23.2 | 738970 | 1.64E−03 | 1.68 |
| OAZ2 | 128694 | 2.70E−03 | 1.42 |
| RGL2 | 741891 | 5.30E−03 | 1.49 |
| IMAGE:666671 | 666671 | 7.53E−03 | 1.33 |
| Transcribed | | | |
| locus | | | |
| PSAT1 | 366388 | 7.99E−03 | 1.52 |
| ETF1 | 146976 | 8.36E−03 | 1.68 |
| IMAGE:687667 | 687667 | 9.11E−03 | 3.863 |
| CDNA | | | |
| clone | | | |
| IMAGE:4811759, | | | |
| partial | | | |
| cds | | | |
| LRPAP1 | 587186 | 9.83E−03 | 2.52 |
| KPNA2 | 667727 | 1.44E−02 | 1.394 |
| IMAGE:669136 | 669136 | 1.55E−02 | 1.55 |
| Transcribed | | | |
| locus, weakly | | | |
| similar to | | | |
| NP_060312.1 | | | |
| hypothetical | | | |
| protein | | | |
| FLJ20489 | | | |
| [Homo | | | |
| sapiens] | | | |
| RBM5 | 744001 | 1.68E−02 | 1.18 |
| PGR1 | 666361 | 1.71E−02 | 1.35 |
| IMAGE:668300 | 668300 | 1.73E−02 | 1.41 |
| Transcribed | | | |
| locus, | | | |
| strongly | | | |
| similar to | | | |
| NP_683708.1 | | | |
| Toll- | | | |
| interleukin 1 | | | |
| receptor | | | |
| domain- | | | |
| containing | | | |
| adaptor | | | |
| protein | | | |
| isoform b; | | | |
| adapter | | | |
| protein wyatt; | | | |
| MyD88 | | | |
| adapter-like | | | |
| protein; TIR | | | |
| domain- | | | |
| containing | | | |
| adapter | | | |
| protein; Toll- | | | |
| interleukin 1 | | | |
| receptor | | | |
| (TIR) | | | |
| domain- | | | |
| containing | | | |
| adaptor | | | |
| protein | | | |
| [Homo | | | |
| sapiens] | | | |
| TM4SF8 | 713647 | 1.76E−02 | 1.43 |
| ICOSLG | 2074228 | 1.79E−02 | 1.47 |
| CA11 | 282587 | 1.82E−02 | 1.52 |
| TPCN1 | 179288 | 1.93E−02 | 1.28 |
| MEF2C | 687270 | 2.09E−02 | 1.45 |
| FLJ10748 | 726830 | 2.10E−02 | 1.744 |
| PDGFD | 666860 | 2.15E−02 | 1.46 |
| IMAGE:687896 | 687896 | 2.32E−02 | 1.28 |
| PREDICTED: | | | |
| Homo | | | |
| sapiens | | | |
| olfactory | | | |
| receptor, | | | |
| family 7, | | | |
| subfamily E, | | | |
| member 31 | | | |
| pseudogene | | | |
| (OR7E31P), | | | |
| mRNA | | | |
| SATB1 | 364510 | 2.96E−02 | 1.28 |
| BCAR1 | 324666 | 3.02E−02 | 1.22 |
| BCL2L11 | 300194 | 3.03E−02 | 1.344 |
| CTNNA1 | 268972 | 3.05E−02 | 1.17 |
| FLJ20254 | 135096 | 3.08E−02 | 1.24 |
| HMGN1 | 565754 | 3.10E−02 | 1.28 |
| TNFSF12 | 136361 | 3.12E−02 | 1.32 |
| CLDN7 | 300268 | 3.31E−02 | 1.59 |
| MGC17299 | 713422 | 3.32E−02 | 1.30 |
| HIVEP1 | 758037 | 3.34E−02 | 1.196 |
| MESDC2 | 713653 | 3.52E−02 | 1.419 |
| PBXIP1 | 366042 | 3.66E−02 | 1.67 |
| SPINT1 | 723914 | 3.90E−02 | 1.55 |
| PDAP1 | 172693 | 3.91E−02 | 1.2 |
| ABCC3 | 208097 | 3.94E−02 | 2.35 |
| APLP2 | 549054 | 4.01E−02 | 1.36 |
| SPATA12 | 730300 | 4.04E−02 | 1.39 |
| FLJ20160 | 175864 | 4.15E−02 | 1.43 |
| FBXL16 | 178908 | 4.37E−02 | 1.45 |
| MGC19604 | 687468 | 4.42E−02 | 1.27 |
| KCNK5 | 134978 | 4.54E−02 | 1.62 |
| IMAGE:729956 | 729956 | 4.63E−02 | 1.244 |
| VEGFC | 503189 | 4.91E−02 | 1.20 |
| IMAGE:681875 | 681875 | 4.91E−02 | 1.29 |

TABLE 11

| | FTA-b | Under | |
|---|---|---|---|
| name | cloid | p-value | fold |
| IFI30 | 740931 | 1.4326E−13 | 0.29 |
| IMAGE:731751 | 731751 | 2.0423E−10 | 0.47 |
| Transcribed | | | |
| locus | | | |
| MUC1 | 840687 | 5.8417E−10 | 0.5 |
| DF | 666128 | 8.9417E−09 | 0.37 |
| C19orf33 | 379540 | 1.0372E−07 | 0.56 |
| MLPH | 667259 | 1.2264E−07 | 0.4 |
| PITPNA | 725585 | 3.9776E−07 | 0.46 |
| NBL1 | 503874 | 8.9123E−07 | 0.39 |
| TIAM1 | 23612 | 1.184E−06 | 0.37 |
| IL2RA | 3054031 | 1.2903E−06 | 0.66 |
| ABCC3 | 208097 | 1.3495E−06 | 0.41 |
| CITED1 | 265558 | 1.4726E−06 | 0.27 |
| IMAGE:136976 | 136976 | 1.7574E−06 | 0.61 |
| Transcribed | | | |
| locus, | | | |
| moderately | | | |
| similar to | | | |
| NP_777603.1 | | | |
| hypothetical | | | |
| protein | | | |
| FLJ25976 | | | |
| [Homo | | | |
| sapiens] | | | |
| APOD | 838611 | 2.6068E−06 | 0.24 |
| RASGRP1 | 725707 | 7.0993E−06 | 0.46 |
| CLDN1 | 594279 | 9.4387E−06 | 0.19 |
| DPP4 | 343987 | 1.0189E−05 | 0.45 |
| NOG | 487474 | 1.3124E−05 | 0.56 |
| FLJ12604 | 731128 | 1.323E−05 | 0.58 |
| AQP4 | 279172 | 1.9121E−05 | 0.59 |
| GPNMB | 773330 | 1.9836E−05 | 0.24 |
| IMAGE:1089025 | 1089025 | 2.3035E−05 | 0.34 |
| GTPBP5 | 725502 | 2.5821E−05 | 0.52 |
| KLRB1 | 2091591 | 2.6472E−05 | 0.63 |
| CD4 | 141216 | 4.1509E−05 | 0.66 |
| MRC2 | 235882 | 4.2346E−05 | 0.52 |
| HCLS1 | 665452 | 4.6839E−05 | 0.61 |
| ATR | 1251197 | 4.6948E−05 | 0.77 |
| SPINL | 236399 | 4.9438E−05 | 0.44 |
| BAI3 | 50491 | 5.2305E−05 | 0.62 |
| LOC90110 | 376699 | 5.4077E−05 | 0.77 |
| IMAGE:727496 | 727496 | 5.6847E−05 | 0.55 |
| Transcribed | | | |
| locus | | | |
| SLC6A8 | 725877 | 6.9226E−05 | 0.32 |
| IRF4 | 682207 | 9.8647E−05 | 0.67 |
| IMAGE:687667 | 687667 | 0.00011938 | 0.4 |
| CDNA | | | |
| clone | | | |
| IMAGE:4811759, | | | |
| partial cds | | | |
| CPXM2 | 729924 | 0.00012549 | 0.6 |
| KCNQ2 | 179534 | 0.00013834 | 0.72 |
| C4A | 724366 | 0.00014538 | 0.6 |
| SYP | 30471 | 0.00014852 | 0.64 |
| FLJ27523 | 743246 | 0.00015423 | 0.39 |
| FABP5 | 1088781 | 0.00018612 | 0.35 |
| CXCL14 | 345034 | 0.00021831 | 0.24 |
| GC | 195340 | 0.00023132 | 0.65 |
| S100A4 | 868577 | 0.00024012 | 0.44 |
| PIK3CA | 2238108 | 0.00025026 | 0.36 |
| WAS | 2148946 | 0.00025378 | 0.66 |
| SPP1 | 258114 | 0.000267 | 0.35 |
| CCL28 | 136919 | 0.00027959 | 0.71 |
| APOE | 1870594 | 0.00028207 | 0.49 |
| ZNF258 | 1241974 | 0.00028771 | 0.74 |
| ALCAM | 172828 | 0.00031982 | 0.66 |
| FLJ37034 | 731348 | 0.00035286 | 0.75 |
| DUSP24 | 740158 | 0.00035953 | 0.49 |
| LASP1 | 592598 | 0.00037148 | 0.43 |
| NCK1 | 1326169 | 0.00038479 | 0.72 |
| ETV4 | 809959 | 0.00038529 | 0.77 |
| AFTIPHILIN | 741790 | 0.00040278 | 0.74 |
| LOC440712 | 725268 | 0.00040484 | 0.65 |
| S100A10 | 119939 | 0.00041115 | 0.64 |
| EGFR | 135980 | 0.000416 | 0.59 |
| IMAGE:136014 | 136014 | 0.00041903 | 0.59 |
| ISG20 | 740604 | 0.00042284 | 0.6 |
| SMARCA5 | 730037 | 0.00047821 | 0.76 |
| SEPT-05 | 448163 | 0.00048532 | 0.24 |
| SCEL | 668239 | 0.00049646 | 0.75 |
| S100A6 | 512420 | 0.00049777 | 0.63 |
| CLECSF12 | 258865 | 0.00055249 | 0.42 |
| CD47 | 365526 | 0.00055731 | 0.74 |
| EED | 667365 | 0.00057991 | 0.76 |
| MAPKAPK2 | 742577 | 0.00059626 | 0.78 |
| PMS2 | 116906 | 0.00062492 | 0.63 |
| IL10RA | 258747 | 0.00078912 | 0.69 |
| IMAGE:738896 | 738896 | 0.00086466 | 0.32 |
| IMAGE:342256 | 342256 | 0.00088142 | 0.28 |
| VDAC1 | 486221 | 0.00088583 | 0.71 |
| SLAMF8 | 288807 | 0.00091298 | 0.67 |
| TPD52 | 743259 | 0.00102858 | 0.21 |
| IMAGE:136223 | 136223 | 0.00102862 | 0.77 |
| BCAP29 | 725970 | 0.00104691 | 0.66 |
| BNIP3 | 342700 | 0.00108019 | 0.72 |
| CCND3 | 327182 | 0.00109024 | 0.79 |
| POPDC3 | 264166 | 0.0012516 | 0.75 |
| CLGN | 1049033 | 0.00129805 | 0.72 |
| SAMD10 | 365801 | 0.00133812 | 0.8 |
| RNASE4 | 156720 | 0.00140105 | 0.74 |
| KLRK1 | 725473 | 0.00158467 | 0.78 |
| BIK | 1074708 | 0.00163982 | 0.77 |
| REPL1 | 290265 | 0.00164775 | 0.83 |
| ANKRD28 | 687379 | 0.00169361 | 0.73 |
| LILRB5 | 71428 | 0.0016981 | 0.75 |
| MATN1 | 1624260 | 0.00177668 | 0.77 |
| ZNF251 | 486401 | 0.00179489 | 0.42 |
| FRMD4B | 669564 | 0.00181652 | 0.64 |
| CAV1 | 133531 | 0.00185645 | 0.68 |
| DCAMKL1 | 232388 | 0.00191401 | 0.72 |
| SALL4 | 726454 | 0.00194797 | 0.53 |
| S100A12 | 1705397 | 0.00201356 | 0.75 |
| CCDC6 | 487848 | 0.00201872 | 0.73 |
| URB | 809719 | 0.00217689 | 0.64 |
| IMAGE:251427 | 251427 | 0.00220137 | 0.74 |
| GRM3 | 287843 | 0.00221023 | 0.75 |
| APOE | 1870594 | 0.00230094 | 0.63 |
| IMAGE:744926 | 744926 | 0.00237853 | 0.64 |
| Transcribed | | | |
| locus | | | |
| HOXD11 | 682119 | 0.00238989 | 0.69 |
| NOTCH2 | 1641901 | 0.00255668 | 0.55 |
| IMAGE:742901 | 742901 | 0.00256267 | 0.78 |
| CCL19 | 1707527 | 0.00265849 | 0.18 |
| IMAGE:742853 | 742853 | 0.00269303 | 0.6 |
| SIAT8B | 33133 | 0.00287355 | 0.82 |
| GSTM2 | 664233 | 0.00328285 | 0.76 |
| SLC39A2 | 504596 | 0.00332451 | 0.78 |
| FLJ35794 | 364204 | 0.00343664 | 0.68 |
| SFRP4 | 285693 | 0.00366218 | 0.56 |
| DOK5 | 25664 | 0.0037568 | 0.67 |
| STX7 | 174396 | 0.0037569 | 0.66 |
| IMAGE:743722 | 743722 | 0.00380385 | 0.78 |
| GABRE | 209137 | 0.00395568 | 0.73 |
| F10 | 310519 | 0.00403123 | 0.74 |
| SAFB | 42280 | 0.00407958 | 0.68 |
| PTPN4 | 249311 | 0.00432638 | 0.8 |
| IL13 | 1505308 | 0.00447394 | 0.74 |
| MAD2L2 | 727178 | 0.00456201 | 0.74 |
| PKNOX1 | 667067 | 0.0045851 | 0.71 |
| IMAGE:731726 | 731726 | 0.00458772 | 0.75 |
| Transcribed | | | |
| locus | | | |
| IMAGE:46991 | 46991 | 0.00461083 | 0.73 |
| Similar | | | |
| to | | | |
| breakpoint | | | |
| cluster | | | |
| region | | | |
| isoform 1 | | | |
| FLJ22794 | 137454 | 0.00464619 | 0.75 |
| PREB | 740347 | 0.00469747 | 0.52 |
| ATP11B | 666334 | 0.00476466 | 0.72 |
| IMAGE:666671 | 666671 | 0.00504158 | 0.81 |
| Transcribed | | | |
| locus | | | |
| NOTCH1 | 359461 | 0.00506296 | 0.71 |
| HSPB7 | 487302 | 0.0052282 | 0.66 |
| CD38 | 1352408 | 0.00526111 | 0.76 |
| PTGES | 504646 | 0.00534239 | 0.81 |
| DKFZp434M202 | 743118 | 0.00543931 | 0.72 |
| STAU | 365919 | 0.00562066 | 0.82 |
| THAP3 | 687800 | 0.00576148 | 0.52 |
| PTPNS1 | 26061 | 0.00606792 | 0.74 |
| ECM1 | 301122 | 0.00616842 | 0.28 |
| RUNX3 | 122874 | 0.00623461 | 0.79 |
| MYC | 129438 | 0.0064463 | 0.63 |
| CUGBP2 | 666235 | 0.00725838 | 0.82 |
| FBXO46 | 471664 | 0.00745485 | 0.73 |
| LCN2 | 741497 | 0.0079592 | 0.35 |
| IMAGE:1117183 | 1117183 | 0.00799012 | 0.79 |
| FMO5 | 364526 | 0.00847507 | 0.79 |
| S100A1 | 175772 | 0.00862472 | 0.65 |
| C10orf6 | 179214 | 0.0086753 | 0.8 |
| HN1 | 471568 | 0.00879178 | 0.74 |
| ZFOC1 | 744606 | 0.00881888 | 0.75 |
| LAMB3 | 1103402 | 0.00904345 | 0.74 |
| PDCD1LG1 | 1942634 | 0.00917818 | 0.79 |
| FAM36A | 727263 | 0.00962448 | 0.77 |
| IMAGE:484577 | 484577 | 0.01003627 | 0.67 |
| Transcribed | | | |
| locus, | | | |
| weakly | | | |
| similar to | | | |
| NP_000991.1 | | | |
| ribosomal | | | |
| protein L39; | | | |
| 60S | | | |
| ribosomal | | | |
| protein L39 | | | |
| [Homo | | | |
| sapiens] | | | |
| TSC | 745490 | 0.01016313 | 0.58 |
| PCSK6 | 31924 | 0.01040107 | 0.7 |
| IGF1R | 682555 | 0.01044883 | 0.78 |
| ATIC | 665693 | 0.01064698 | 0.83 |
| CASP3 | 823680 | 0.01086953 | 0.72 |
| SLAMF1 | 1626951 | 0.01116187 | 0.81 |
| STK17A | 562904 | 0.01118617 | 0.76 |
| IMAGE:682311 | 682311 | 0.01131518 | 0.81 |
| LOC440722 | | | |
| AMD1 | 132752 | 0.01141962 | 0.81 |
| TFRC | 359797 | 0.01168045 | 0.78 |
| RGS19 | 485803 | 0.01210737 | 0.79 |
| HLA-DPB1 | 725548 | 0.01225207 | 0.71 |
| RBBP4 | 705147 | 0.01232497 | 0.78 |
| INHBB | 730012 | 0.01243708 | 0.67 |
| IMAGE:504539 | 504539 | 0.01255589 | 0.77 |
| C4A | 491004 | 0.01259752 | 0.71 |
| PARP9 | 667440 | 0.0127547 | 0.82 |
| CD33 | 1917430 | 0.01352763 | 0.73 |
| IMP-2 | 743774 | 0.01360949 | 0.51 |
| CENTD1 | 666110 | 0.01509604 | 0.78 |
| ALDH1A1 | 309697 | 0.01556687 | 0.68 |
| IMAGE:724416 | 724416 | 0.01642786 | 0.7 |
| Transcribed | | | |
| locus | | | |
| HT007 | 135303 | 0.01657774 | 0.83 |
| MGC29784 | 731019 | 0.01673005 | 0.87 |
| MAP2 | 1287638 | 0.01674651 | 0.7 |
| LOC253981 | 742743 | 0.0168504 | 0.75 |
| LOC389906 | 740718 | 0.01771548 | 0.85 |
| PDK1 | 1645668 | 0.01800445 | 0.84 |
| SLPI | 378813 | 0.01811761 | 0.62 |
| DEK | 1016390 | 0.0200655 | 0.81 |
| VIL2 | 124701 | 0.02020796 | 0.75 |
| TIMP1 | 162246 | 0.02061013 | 0.57 |
| RFX2 | 731738 | 0.0206857 | 0.69 |
| C4orf16 | 681890 | 0.02095375 | 0.82 |
| CAV1 | 309645 | 0.0222001 | 0.64 |
| PDCD4 | 665376 | 0.02257053 | 0.72 |
| MGC5395 | 238840 | 0.02292419 | 0.83 |
| CERKL | 489326 | 0.0238201 | 0.77 |
| APOL1 | 665632 | 0.02452738 | 0.63 |
| PLCG2 | 201467 | 0.02564117 | 0.8 |
| KLF4 | 188232 | 0.02771743 | 0.71 |
| ALDH1A3 | 486189 | 0.02811344 | 0.69 |
| GFPT2 | 485085 | 0.02897134 | 0.82 |
| ACTB | 203166 | 0.02910806 | 0.76 |
| DKFZP564D166 | 744374 | 0.02968329 | 0.85 |
| TOSO | 813174 | 0.0306376 | 0.79 |
| CD8B1 | 1743279 | 0.03083639 | 0.84 |
| FCHO1 | 742763 | 0.03087022 | 0.83 |
| ICAM3 | 156183 | 0.03162562 | 0.85 |
| EPRS | 669549 | 0.03174483 | 0.85 |
| ST5 | 376356 | 0.03189569 | 0.86 |
| DEFB1 | 665086 | 0.03223041 | 0.57 |
| CCL13 | 2449149 | 0.0324626 | 0.79 |
| MAN1A1 | 137719 | 0.03287328 | 0.66 |
| IL2RB | 139073 | 0.0337379 | 0.84 |
| CD47 | 357442 | 0.03389369 | 0.77 |
| USP18 | 745083 | 0.03455337 | 0.8 |
| IMAGE:724544 | 724544 | 0.03490328 | 0.73 |
| Similar | | | |
| to 40S | | | |
| ribosomal | | | |
| protein S25 | | | |
| IMAGE:132933 | 132933 | 0.03499764 | 0.8 |
| C20orf18 | 665433 | 0.03600838 | 0.73 |
| IMAGE:2497617 | 2497617 | 0.03637846 | 0.81 |
| LIPT1 | 22085 | 0.03746726 | 0.82 |
| NSUN4 | 364570 | 0.03889642 | 0.76 |
| MX1 | 713879 | 0.04036441 | 0.8 |
| WDR5 | 731023 | 0.0411845 | 0.82 |
| GSTM2 | 713922 | 0.04311433 | 0.84 |
| LOC120224 | 252291 | 0.04339193 | 0.73 |
| CAV1 | 1911930 | 0.04340567 | 0.79 |
| MPO | 436554 | 0.04449178 | 0.86 |
| IMAGE:743415 | 743415 | 0.0451026 | 0.86 |
| Transcribed | | | |
| locus | | | |
| KLRD1 | 145696 | 0.04603368 | 0.85 |
| ATF4 | 178348 | 0.04624908 | 0.84 |
| G3BP | 667239 | 0.04690026 | 0.84 |
| ELK4 | 236155 | 0.04765789 | 0.84 |
| AKR1C1 | 196992 | 0.04840514 | 0.45 |
| IKBKB | 2484742 | 0.04904713 | 0.87 |
| GATA4 | 781738 | 0.04957556 | 0.83 |
| SEMA4D | 210587 | 0.04995642 | 0.79 |

TABLE 12

| | FTA-b | Over | |
|---|---|---|---|
| name | cloid | p-value | fold |
| PLXNC1 | 724609 | 1.13E−03 | 1.92 |
| IMAGE:365913 | 365913 | 1.44E−03 | 1.54 |
| Transcribed | | | |
| locus | | | |
| PDE8A | 666154 | 1.47E−03 | 1.30 |
| SPARC | 267358 | 2.52E−03 | 1.56 |
| C6orf85 | 665379 | 2.79E−03 | 1.25 |
| DCI | 667892 | 3.32E−03 | 1.88 |
| TMED4 | 251250 | 5.09E−03 | 1.46 |
| MESDC2 | 713653 | 6.21E−03 | 1.44 |
| FLJ45459 | 365589 | 6.88E−03 | 1.35 |
| ID1 | 1087348 | 7.35E−03 | 1.71 |
| PBXIP1 | 366042 | 7.53E−03 | 1.41 |
| FAM38B | 743146 | 0.00757 | 1.275 |
| KNS2 | 174654 | 0.00785 | 1.564 |
| LMLN | 744657 | 8.03E−03 | 1.36 |
| CDH1 | 214008 | 9.02E−03 | 1.48 |
| USP13 | 666007 | 9.33E−03 | 1.57 |
| MGC3329 | 137005 | 0.00969 | 1.297 |
| PITPNC1 | 364436 | 1.06E−02 | 1.56 |
| TEX13A | 738558 | 1.12E−02 | 1.33 |
| ETF1 | 146976 | 0.01148 | 1.526 |
| FLJ12681 | 682175 | 1.15E−02 | 1.33 |
| MGC5178 | 725533 | 0.01202 | 1.32 |
| EMX2 | 365121 | 1.22E−02 | 1.46 |
| C9orf119 | 667112 | 1.24E−02 | 1.39 |
| CD34 | 770858 | 1.25E−02 | 1.43 |
| S100A2 | 3659591 | 1.31E−02 | 1.44 |
| KDR | 469345 | 0.01314 | 1.341 |
| FCGBP | 154172 | 1.37E−02 | 1.52 |
| LOC340073 | 731404 | 1.45E−02 | 1.56 |
| MXD4 | 730036 | 1.57E−02 | 1.22 |
| ITGA5 | 135671 | 1.59E−02 | 1.31 |
| MGC99813 | 739097 | 1.63E−02 | 2.20 |
| CRABP1 | 739193 | 1.63E−02 | 2.53 |
| IMAGE:743205 | 743205 | 1.65E−02 | 1.41 |
| GOLPH2 | 811582 | 1.71E−02 | 1.33 |
| FLJ13491 | 364352 | 1.85E−02 | 1.33 |
| PVRL2 | 725364 | 1.86E−02 | 1.50 |
| IMAGE:668684 | 668684 | 2.04E−02 | 1.31 |
| Transcribed | | | |
| locus, weakly | | | |
| similar to | | | |
| XP_209041.2 | | | |
| PREDICTED: | | | |
| similar to | | | |
| KIAA1503 | | | |
| protein [Homo | | | |
| sapiens] | | | |
| NT5C2 | 725076 | 0.02133 | 1.281 |
| IMAGE:666946 | 666946 | 2.15E−02 | 1.28 |
| IMMP2L | 136260 | 2.22E−02 | 1.17 |
| RAP1GA1 | 971276 | 2.24E−02 | 1.54 |
| BRAF | 2139164 | 2.26E−02 | 1.29 |
| GLT8D2 | 365271 | 2.28E−02 | 2.01 |
| RANBP10 | 307025 | 2.29E−02 | 1.20 |
| GLG1 | 366254 | 2.33E−02 | 1.35 |
| CD63 | 125552 | 2.35E−02 | 1.30 |
| SGSH | 669263 | 2.39E−02 | 1.36 |
| IMAGE:744439 | 744439 | 2.63E−02 | 1.26 |
| Transcribed | | | |
| locus | | | |
| LRRC28 | 26519 | 2.69E−02 | 1.38 |
| GRB10 | 564994 | 2.78E−02 | 1.48 |
| FLT4 | 668815 | 2.93E−02 | 1.26 |
| DCTN2 | 725335 | 3.12E−02 | 1.17 |
| KRT7 | 592276 | 3.21E−02 | 1.72 |
| IMAGE:176543 | 176543 | 3.34E−02 | 1.19 |
| DIO1 | 296702 | 3.37E−02 | 1.57 |
| RGS20 | 668152 | 3.44E−02 | 1.27 |
| RAD51L1 | 295412 | 0.0345 | 1.565 |
| GATA2 | 149809 | 3.60E−02 | 1.26 |
| RGS6 | 24176 | 3.62E−02 | 1.44 |
| CES2 | 153667 | 3.67E−02 | 1.32 |
| FLJ44005 | 726675 | 3.73E−02 | 1.42 |
| CDH13 | 486510 | 3.88E−02 | 1.43 |
| GCLC | 1526058 | 4.06E−02 | 1.17 |
| C2orf7 | 366243 | 4.50E−02 | 1.27 |
| GATA3 | 148796 | 4.51E−02 | 1.31 |
| TSG101 | 194350 | 4.52E−02 | 1.16 |
| CKLFSF4 | 143759 | 4.59E−02 | 1.24 |
| KIAA0789 | 33621 | 4.59E−02 | 1.30 |
| ZNF580 | 668089 | 4.90E−02 | 1.28 |
| PIGV | 669319 | 5.00E−02 | 1.16 |

TABLE 13

| | FTC | Under | |
|---|---|---|---|
| name | cloid | p-value | fold |
| TIMP3 | 501476 | 3.27118E−28 | 0.255 |
| MDK | 309009 | 4.17066E−17 | 0.216 |
| CRABP1 | 739193 | 1.13484E−16 | 0.071 |
| ARMCX3 | 251452 | 3.43628E−15 | 0.346 |
| ETHE1 | 681957 | 3.82022E−15 | 0.593 |
| MGC99813 | 739097 | 2.29648E−14 | 0.14 |
| IKBKE | 364448 | 6.52667E−14 | 0.703 |
| HBB | 469549 | 1.08191E−10 | 0.196 |
| LILRB2 | 202897 | 1.82324E−10 | 0.477 |
| GC | 195340 | 7.63448E−10 | 0.467 |
| ARHGAP10 | 731380 | 1.46351E−09 | 0.6 |
| CCR7 | 2345206 | 4.3297E−09 | 0.569 |
| IMAGE:23765 | 23765 | 5.53388E−09 | 0.55 |
| RODH | 471641 | 2.47866E−08 | 0.243 |
| TREM2 | 502244 | 2.75199E−08 | 0.682 |
| MESDC2 | 713653 | 6.65348E−08 | 0.465 |
| ALDH1A3 | 486189 | 1.02206E−07 | 0.631 |
| ISG20 | 740604 | 1.21406E−07 | 0.623 |
| IRF4 | 682207 | 1.50961E−07 | 0.558 |
| C10orf116 | 740941 | 1.82217E−07 | 0.355 |
| HBA2 | 469647 | 2.06716E−07 | 0.185 |
| DPF2 | 743519 | 2.65982E−07 | 0.768 |
| NCB5OR | 743367 | 4.04597E−07 | 0.441 |
| TRAF2 | 966894 | 9.00279E−07 | 0.652 |
| SYN3 | 727056 | 1.0981E−06 | 0.303 |
| BCAP29 | 2336358 | 2.65105E−06 | 0.523 |
| TOE1 | 52897 | 3.47029E−06 | 0.682 |
| NCK1 | 1326169 | 4.11032E−06 | 0.655 |
| GPNMB | 773330 | 4.11149E−06 | 0.191 |
| IL2RB | 139073 | 6.44261E−06 | 0.742 |
| HLA- | 725548 | 6.72755E−06 | 0.448 |
| DPB1 | | | |
| ANXA11 | 137238 | 9.73001E−06 | 0.794 |
| IMAGE:742853 | 742853 | 1.43601E−05 | 0.464 |
| APOD | 838611 | 1.96792E−05 | 0.267 |
| IMAGE:22908 | 22908 | 2.94694E−05 | 0.524 |
| Transcribed | | | |
| locus, | | | |
| weakly | | | |
| similar to | | | |
| NP_775735.1 | | | |
| I(3)mbt- | | | |
| like 4 | | | |
| [Homo | | | |
| sapiens] | | | |
| SLC17A2 | 207920 | 3.13767E−05 | 0.278 |
| WNT4 | 375746 | 5.12808E−05 | 0.783 |
| IMAGE:738896 | 738896 | 5.38726E−05 | 0.17 |
| PLA2G2A | 152802 | 5.92307E−05 | 0.61 |
| DPP4 | 343987 | 6.26833E−05 | 0.495 |
| FLJ27523 | 743246 | 8.22724E−05 | 0.347 |
| IMAGE:1089025 | 1089025 | 8.66321E−05 | 0.405 |
| IMAGE:342256 | 342256 | 9.00293E−05 | 0.171 |
| CPXM2 | 729924 | 9.0502E−05 | 0.379 |
| IMAGE:727496 | 727496 | 0.000107885 | 0.569 |
| Transcribed | | | |
| locus | | | |
| TP53I11 | 667514 | 0.000107922 | 0.46 |
| BAI3 | 50491 | 0.00010801 | 0.563 |
| SALL4 | 726454 | 0.000203015 | 0.336 |
| ZNF251 | 486401 | 0.000204182 | 0.342 |
| PIK3CA | 2238108 | 0.000241673 | 0.316 |
| FLJ12903 | 730123 | 0.000273124 | 0.686 |
| HIS1 | 342551 | 0.000280435 | 0.52 |
| BCL2L12 | 1186334 | 0.000312735 | 0.752 |
| FLJ45459 | 365589 | 0.000371257 | 0.554 |
| ABCB1 | 1837488 | 0.000377025 | 0.682 |
| PDCD1LG1 | 1942634 | 0.000469177 | 0.785 |
| CD69 | 276727 | 0.000475157 | 0.514 |
| FLJ30707 | 666986 | 0.000530185 | 0.889 |
| YWHAH | 324066 | 0.000614917 | 0.701 |
| SCMH1 | 724014 | 0.000705203 | 0.557 |
| AXL | 364083 | 0.000726257 | 0.397 |
| CCL13 | 2449149 | 0.000733565 | 0.665 |
| FLJ14351 | 744943 | 0.00080337 | 0.391 |
| IL10RA | 258747 | 0.000976141 | 0.564 |
| IGHM | 276658 | 0.00121878 | 0.633 |
| CITED1 | 265558 | 0.0012369 | 0.487 |
| C6orf118 | 731745 | 0.001239503 | 0.705 |
| CDH16 | 726763 | 0.001947806 | 0.389 |
| SYT1 | 28511 | 0.002006575 | 0.618 |
| MRC2 | 235882 | 0.00205901 | 0.452 |
| CCL19 | 1707527 | 0.002223263 | 0.182 |
| HDAC1 | 1896337 | 0.002482715 | 0.694 |
| SEPT-05 | 448163 | 0.002647911 | 0.341 |
| ECM1 | 301122 | 0.002741544 | 0.224 |
| GNAI2 | 724306 | 0.002774431 | 0.893 |
| THAP3 | 687800 | 0.002955378 | 0.496 |
| DEPDC6 | 669318 | 0.002997235 | 0.368 |
| PTHLH | 2214396 | 0.003118854 | 0.674 |
| IMAGE:175268 | 175268 | 0.003169415 | 0.477 |
| PREB | 740347 | 0.003182387 | 0.355 |
| GLT8D2 | 365271 | 0.003312971 | 0.507 |
| LOH12CR2 | 366074 | 0.003465031 | 0.706 |
| CLDN1 | 594279 | 0.003571509 | 0.327 |
| PDCD1LG1 | 1942634 | 0.003641855 | 0.603 |
| APOL3 | 366289 | 0.003733369 | 0.709 |
| DEFB1 | 665086 | 0.003754037 | 0.44 |
| NOTCH2 | 1641901 | 0.00433718 | 0.516 |
| C4A | 724366 | 0.005027025 | 0.419 |
| COX8A | 509606 | 0.006006104 | 0.469 |
| PTPNS1 | 26061 | 0.006559782 | 0.53 |
| TIMP1 | 162246 | 0.006602902 | 0.333 |
| CKLFSF3 | 489249 | 0.006843773 | 0.753 |
| ETV1 | 24541 | 0.007109204 | 0.767 |
| SPON2 | 723923 | 0.00735775 | 0.628 |
| IMAGE:731751 | 731751 | 0.007589669 | 0.548 |
| Transcribed | | | |
| locus | | | |
| IMAGE:744410 | 744410 | 0.00769332 | 0.779 |
| Transcribed | | | |
| locus | | | |
| APOE | 1870594 | 0.007740935 | 0.323 |
| TPD52 | 743259 | 0.007753957 | 0.352 |
| HMG20A | 731277 | 0.007809821 | 0.564 |
| GALNT7 | 381854 | 0.008046311 | 0.836 |
| GPR108 | 25949 | 0.008356628 | 0.56 |
| CERKL | 489326 | 0.008740448 | 0.482 |
| C4A | 491004 | 0.009053823 | 0.445 |
| KLRD1 | 2728204 | 0.009193925 | 0.702 |
| OAS1 | 666703 | 0.009198225 | 0.586 |
| FLJ21159 | 251147 | 0.00940131 | 0.668 |
| APOE | 1870594 | 0.009962428 | 0.454 |
| AOC3 | 484535 | 0.010034148 | 0.634 |
| SPP1 | 258114 | 0.010053336 | 0.457 |
| LRRC28 | 26519 | 0.011941366 | 0.387 |
| WIPI49 | 487148 | 0.012228545 | 0.735 |
| LAPTM4A | 726684 | 0.012475634 | 0.269 |
| GATA2 | 149809 | 0.012854907 | 0.758 |
| IMAGE:731616 | 731616 | 0.013356612 | 0.415 |
| MRNA; | | | |
| cDNA | | | |
| DKFZp686A102 | | | |
| (from | | | |
| clone | | | |
| DKFZp686A102) | | | |
| LOC120224 | 252291 | 0.014999368 | 0.594 |
| LILRB5 | 71428 | 0.016005286 | 0.745 |
| PRUNE | 364324 | 0.016566051 | 0.608 |
| ICAM3 | 156183 | 0.016831639 | 0.649 |
| MESP1 | 25865 | 0.017300239 | 0.735 |
| S100A12 | 1705397 | 0.017625347 | 0.606 |
| TMED4 | 251250 | 0.017788466 | 0.307 |
| MAP7 | 79729 | 0.01797944 | 0.572 |
| IMAGE:365562 | 365562 | 0.018131317 | 0.77 |
| Transcribed | | | |
| locus, | | | |
| moderately | | | |
| similar to | | | |
| XP_521389.1 | | | |
| PREDICTED: | | | |
| similar to | | | |
| UTY [Pan | | | |
| troglodytes] | | | |
| BST2 | 811024 | 0.018268156 | 0.73 |
| OBSCN | 730926 | 0.019249071 | 0.661 |
| COX5A | 1085884 | 0.020164977 | 0.667 |
| RNF123 | 366159 | 0.021646555 | 0.853 |
| IGSF10 | 682276 | 0.022377266 | 0.753 |
| NDUFA7 | 364469 | 0.024164639 | 0.48 |
| DDR2 | 668442 | 0.024237373 | 0.566 |
| TACC1 | 741905 | 0.02441973 | 0.496 |
| CD5 | 356841 | 0.024474896 | 0.636 |
| C21orf84 | 730814 | 0.025612809 | 0.747 |
| SYT7 | 177827 | 0.02577182 | 0.587 |
| SAFB | 42280 | 0.025773596 | 0.765 |
| MTAP | 724151 | 0.026004214 | 0.763 |
| BIC | 743270 | 0.026405121 | 0.738 |
| IMMP2L | 136260 | 0.026660518 | 0.536 |
| GFPT2 | 485085 | 0.026990136 | 0.741 |
| IMAGE:742837 | 742837 | 0.027538211 | 0.83 |
| Transcribed | | | |
| locus | | | |
| DF | 666128 | 0.028156903 | 0.452 |
| IMAGE:666279 | 666279 | 0.029379728 | 0.624 |
| CDNA | | | |
| FLJ30779 | | | |
| fis, clone | | | |
| FEBRA2000815 | | | |
| RYK | 727092 | 0.029455425 | 0.728 |
| PPIL4 | 364777 | 0.02990287 | 0.314 |
| TJP1 | 179334 | 0.030457336 | 0.563 |
| CAPN3 | 757248 | 0.030652263 | 0.681 |
| IMAGE:2484270 | 2484270 | 0.031332245 | 0.707 |
| GRM3 | 287843 | 0.031389431 | 0.727 |
| KDR | 469345 | 0.031468457 | 0.521 |
| ADCY3 | 667061 | 0.031634804 | 0.526 |
| RGS19 | 485803 | 0.032147875 | 0.497 |
| IMAGE:727491 | 727491 | 0.034722498 | 0.364 |
| CXCL12 | 213113 | 0.035745974 | 0.694 |
| FLJ12998 | 364846 | 0.036806278 | 0.576 |
| IMAGE:743619 | 743619 | 0.036830771 | 0.726 |
| ARL6IP2 | 743987 | 0.037296129 | 0.493 |
| DCTN1 | 180640 | 0.037381682 | 0.554 |
| APIN | 364706 | 0.038549022 | 0.62 |
| FLJ32731 | 365177 | 0.039009106 | 0.77 |
| LASP1 | 592598 | 0.039649505 | 0.594 |
| RAP1GA1 | 971276 | 0.040572673 | 0.687 |
| VAV2 | 1581686 | 0.042133135 | 0.748 |
| FLJ34433 | 713671 | 0.042784525 | 0.821 |
| MYC | 129438 | 0.043192437 | 0.578 |
| IMAGE:1117183 | 1117183 | 0.043871839 | 0.671 |
| CXCL14 | 345034 | 0.044460416 | 0.292 |
| ZNF250 | 741066 | 0.044484132 | 0.419 |
| TRAF5 | 1286238 | 0.044516219 | 0.635 |
| SYNPO | 178792 | 0.044836883 | 0.64 |
| IFI30 | 740931 | 0.045498386 | 0.616 |
| LMLN | 744657 | 0.046068298 | 0.648 |
| TUSC1 | 740381 | 0.046224198 | 0.898 |
| EPHA4 | 2237263 | 0.046590574 | 0.784 |
| PDE8A | 666154 | 0.047026631 | 0.555 |
| ARID1B | 665538 | 0.047648823 | 0.631 |
| ITGA5 | 135671 | 0.048291346 | 0.699 |
| CREBBP | 172996 | 0.048559519 | 0.817 |
| NF2 | 1698236 | 0.048577039 | 0.629 |
| DNMT1 | 768241 | 0.049510935 | 0.69 |
| POU2F2 | 188393 | 0.049542968 | 0.619 |

TABLE 14

| | FTC | Over | |
|---|---|---|---|
| name | cloid | p-value | fold |
| DNALI1 | 782688 | 5.13E−31 | 1.83 |
| IMAGE:136014 | 136014 | 4.35E−09 | 1.73 |
| EGFR | 135980 | 4.38E−09 | 1.73 |
| DCTN2 | 725335 | 4.32E−08 | 1.39 |
| HSPB1 | 724150 | 9.31E−07 | 1.59 |
| GCAT | 307094 | 3.74E−06 | 1.29 |
| IMAGE:723752 | 723752 | 1.88E−05 | 1.34 |
| GRB7 | 510318 | 2.89E−05 | 1.35 |
| IQCF3 | 726578 | 2.42E−04 | 2.43 |
| CCND1 | 324079 | 2.91E−04 | 2.47 |
| BRAF | 2139164 | 3.09E−04 | 1.28 |
| CKLFSF4 | 143759 | 4.45E−04 | 1.64 |
| SENP6 | 739237 | 7.42E−04 | 1.77 |
| CAPNS1 | 152655 | 7.56E−04 | 1.31 |
| NY-REN-41 | 136646 | 4.34E−03 | 1.43 |
| ALDH1A1 | 309697 | 6.65E−03 | 1.61 |
| IMAGE:384087 | 384087 | 0.01 | 1.39 |
| DGKD | 365408 | 1.14E−02 | 2.21 |
| LOC90110 | 376699 | 1.16E−02 | 1.42 |
| DNCLI2 | 134671 | 1.19E−02 | 2.26 |
| BAZ2B | 609631 | 1.21E−02 | 1.74 |
| MGC5395 | 238840 | 1.46E−02 | 2.41 |
| BIK | 1074708 | 1.53E−02 | 1.12 |
| GLG1 | 366254 | 1.63E−02 | 1.62 |
| IMAGE:742919 | 742919 | 1.72E−02 | 1.81 |
| Transcribed | | | |
| locus | | | |
| PPP1CA | 257259 | 2.09E−02 | 1.41 |
| CCL5 | 840753 | 2.37E−02 | 1.90 |
| IMAGE:668924 | 668924 | 2.58E−02 | 1.43 |
| IMAGE:136801 | 136801 | 2.72E−02 | 2.18 |
| GGN | 743245 | 2.96E−02 | 1.11 |
| IMAGE:731428 | 731428 | 3.12E−02 | 1.54 |
| Transcribed | | | |
| locus, weakly | | | |
| similar to | | | |
| XP_521389.1 | | | |
| PREDICTED: | | | |
| similar to | | | |
| UTY [Pan | | | |
| troglodytes] | | | |
| DKFZp547K054 | 731410 | 3.31E−02 | 1.93 |
| AGPAT3 | 738507 | 3.71E−02 | 1.55 |
| RTN3 | 430263 | 3.77E−02 | 1.80 |
| DKFZP564D166 | 744374 | 3.79E−02 | 1.36 |
| IL13RA1 | 1492440 | 3.81E−02 | 1.32 |
| AXIN2 | 135887 | 3.84E−02 | 1.84 |
| IMAGE:726782 | 726782 | 4.33E−02 | 2.26 |
| Transcribed | | | |
| locus | | | |
| COL27A1 | 379517 | 4.47E−02 | 1.29 |
| C9orf72 | 726849 | 4.95E−02 | 2.25 |

TABLE 15

| | MNG | Under | |
|---|---|---|---|
| name | cloid | p-value | Fold |
| ABCA8 | 284828 | 1.79E−06 | 0.62 |
| CACNB2 | 173841 | 2.60E−06 | 0.59 |
| IMAGE:667527 | 667527 | 9.53E−06 | 0.63 |
| LILRB2 | 202897 | 9.61E−06 | 0.54 |
| HBB | 469549 | 1.12E−05 | 0.38 |
| SLC17A2 | 207920 | 1.30E−05 | 0.45 |
| HBA2 | 469647 | 1.46E−05 | 0.4 |
| IMAGE:363955 | 363955 | 1.70E−05 | 0.6 |
| Transcribed | | | |
| locus | | | |
| MAPK3 | 323438 | 1.81E−05 | 0.65 |
| IGHM | 276658 | 1.96E−05 | 0.77 |
| RUNX1 | 263251 | 3.29E−05 | 0.72 |
| HMG20A | 731277 | 4.64E−05 | 0.68 |
| JUP | 35628 | 5.75E−05 | 0.59 |
| GPI | 741474 | 6.80E−05 | 0.72 |
| DMD | 743394 | 6.85E−05 | 0.67 |
| ABCC5 | 212366 | 7.48E−05 | 0.78 |
| CLECSF12 | 258865 | 7.61E−05 | 0.41 |
| SCMH1 | 724014 | 9.16E−05 | 0.59 |
| IMAGE:742837 | 742837 | 1.15E−04 | 0.71 |
| Transcribed | | | |
| locus | | | |
| IMAGE:1117183 | 1117183 | 1.20E−04 | 0.75 |
| CD69 | 276727 | 1.54E−04 | 0.73 |
| GOLPH2 | 811582 | 1.55E−04 | 0.76 |
| CD8B1 | 1743279 | 1.56E−04 | 0.77 |
| IMAGE:666279 | 666279 | 1.77E−04 | 0.6 |
| CDNA | | | |
| FLJ30779 fis, | | | |
| clone | | | |
| FEBRA2000815 | | | |
| OAS1 | 666703 | 1.85E−04 | 0.65 |
| PITPNA | 725585 | 1.98E−04 | 0.59 |
| TRIM8 | 714498 | 2.67E−04 | 0.78 |
| ALDH3A1 | 525221 | 2.90E−04 | 0.66 |
| AP2S1 | 739109 | 2.94E−04 | 0.79 |
| ARHGAP10 | 731380 | 3.14E−04 | 0.65 |
| ANP32E | 382738 | 3.79E−04 | 0.68 |
| BAP1 | 1012990 | 4.47E−04 | 0.7 |
| KLF4 | 188232 | 5.18E−04 | 0.58 |
| ABR | 1012903 | 5.18E−04 | 0.77 |
| FLJ44005 | 726675 | 5.89E−04 | 0.79 |
| CASP4 | 356960 | 6.18E−04 | 0.76 |
| MRC2 | 235882 | 6.38E−04 | 0.56 |
| PRDX2 | 208439 | 6.58E−04 | 0.76 |
| ATF4 | 178348 | 7.17E−04 | 0.76 |
| SYN3 | 727056 | 7.28E−04 | 0.58 |
| SQLE | 124781 | 7.44E−04 | 0.72 |
| CLDN7 | 300268 | 7.48E−04 | 0.76 |
| S100A4 | 868577 | 7.51E−04 | 0.46 |
| CCND3 | 327182 | 7.62E−04 | 0.81 |
| PTPN4 | 249311 | 8.22E−04 | 0.79 |
| MLANA | 266361 | 8.46E−04 | 0.78 |
| CASP10 | 241481 | 8.58E−04 | 0.68 |
| ADAMTS9 | 376153 | 9.08E−04 | 0.71 |
| TSC | 745490 | 1.10E−03 | 0.53 |
| PCSK6 | 31924 | 1.15E−03 | 0.67 |
| BAK1 | 235938 | 1.17E−03 | 0.7 |
| IMAGE:687667 | 687667 | 1.19E−03 | 0.48 |
| CDNA | | | |
| clone | | | |
| IMAGE:4811759, | | | |
| partial | | | |
| cds | | | |
| TU3A | 44881 | 1.21E−03 | 0.72 |
| HLA-DPB1 | 725548 | 1.22E−03 | 0.67 |
| MAPRE2 | 383868 | 1.35E−03 | 0.64 |
| HIS1 | 342551 | 1.38E−03 | 0.75 |
| IMAGE:382521 | 382521 | 1.41E−03 | 0.66 |
| KPNA2 | 667727 | 1.55E−03 | 0.76 |
| IMAGE:365562 | 365562 | 1.75E−03 | 0.8 |
| Transcribed | | | |
| locus, | | | |
| moderately | | | |
| similar to | | | |
| XP_521389.1 | | | |
| PREDICTED: | | | |
| similar to | | | |
| UTY [Pan | | | |
| troglodytes] | | | |
| CDH3 | 359051 | 1.76E−03 | 0.61 |
| SYCP2 | 136863 | 1.80E−03 | 0.71 |
| SLC9A3R1 | 773286 | 1.81E−03 | 0.74 |
| ELK4 | 236155 | 1.81E−03 | 0.74 |
| IMAGE:744899 | 744899 | 1.90E−03 | 0.77 |
| Homo | | | |
| sapiens, | | | |
| clone | | | |
| IMAGE:4837072, | | | |
| mRNA | | | |
| PBXIP1 | 366042 | 1.92E−03 | 0.81 |
| PSMD11 | 383945 | 2.05E−03 | 0.72 |
| SPON2 | 723923 | 2.08E−03 | 0.76 |
| LEFTY2 | 340657 | 2.08E−03 | 0.74 |
| DDR2 | 668442 | 2.10E−03 | 0.78 |
| GNAS | 382791 | 2.15E−03 | 0.64 |
| IMAGE:666794 | 666794 | 2.17E−03 | 0.74 |
| Hypothetical | | | |
| LOC388170 | | | |
| LAD1 | 121551 | 2.17E−03 | 0.8 |
| SFN | 346610 | 2.19E−03 | 0.8 |
| IMAGE:232366 | 232366 | 2.20E−03 | 0.73 |
| CDNA | | | |
| FLJ37672 fis, | | | |
| clone | | | |
| BRHIP2012059 | | | |
| PPP3CC | 110481 | 2.27E−03 | 0.79 |
| BID | 128065 | 2.27E−03 | 0.79 |
| IMAGE:379937 | 379937 | 2.31E−03 | 0.72 |
| FOXN4 | 731076 | 2.31E−03 | 0.78 |
| IRF4 | 682207 | 2.41E−03 | 0.69 |
| SNCB | 50202 | 2.45E−03 | 0.7 |
| ANKRD32 | 725198 | 2.56E−03 | 0.79 |
| KLF1 | 208991 | 2.66E−03 | 0.69 |
| C1QBP | 173371 | 2.67E−03 | 0.75 |
| DC12 | 724895 | 2.73E−03 | 0.76 |
| FCHO1 | 742763 | 2.82E−03 | 0.79 |
| CLNS1A | 666426 | 2.83E−03 | 0.8 |
| PDXP | 743182 | 2.87E−03 | 0.81 |
| GALNT7 | 381854 | 2.90E−03 | 0.73 |
| MAP2 | 347503 | 2.96E−03 | 0.75 |
| IMAGE:743903 | 743903 | 3.01E−03 | 0.6 |
| Homo | | | |
| sapiens, | | | |
| clone | | | |
| IMAGE:5266541, | | | |
| mRNA | | | |
| APOD | 838611 | 3.05E−03 | 0.47 |
| IGKV1-5 | 155345 | 3.08E−03 | 0.79 |
| JAK1 | 2030501 | 3.16E−03 | 0.76 |
| BST2 | 811024 | 3.20E−03 | 0.8 |
| CERKL | 489326 | 3.27E−03 | 0.71 |
| AD023 | 731645 | 3.45E−03 | 0.82 |
| EMILIN2 | 365521 | 3.45E−03 | 0.82 |
| ITGA5 | 135671 | 3.46E−03 | 0.76 |
| PSG3 | 136747 | 3.67E−03 | 0.74 |
| C20orf18 | 665433 | 3.69E−03 | 0.7 |
| S100A12 | 1705397 | 3.71E−03 | 0.78 |
| IMAGE:731689 | 731689 | 3.80E−03 | 0.82 |
| Transcribed | | | |
| locus | | | |
| PLXNC1 | 724609 | 4.01E−03 | 0.77 |
| IMAGE:740105 | 740105 | 4.31E−03 | 0.77 |
| CCDC7 | 731389 | 4.33E−03 | 0.74 |
| BAK1 | 1288183 | 4.52E−03 | 0.62 |
| RAD51C | 80162 | 4.75E−03 | 0.83 |
| KIAA0427 | 127507 | 4.83E−03 | 0.85 |
| MAK | 382002 | 4.94E−03 | 0.76 |
| DEFB1 | 665086 | 4.98E−03 | 0.44 |
| CXCL12 | 213113 | 5.21E−03 | 0.82 |
| CD4 | 141216 | 5.35E−03 | 0.72 |
| LEF1 | 713913 | 5.36E−03 | 0.76 |
| TOSO | 813174 | 5.39E−03 | 0.73 |
| C21orf7 | 665495 | 5.69E−03 | 0.77 |
| SIAT8F | 667110 | 6.23E−03 | 0.73 |
| MTUS1 | 744395 | 6.27E−03 | 0.61 |
| IMAGE:668300 | 668300 | 6.39E−03 | 0.83 |
| Transcribed | | | |
| locus, | | | |
| strongly | | | |
| similar to | | | |
| NP_683708.1 | | | |
| Toll- | | | |
| interleukin 1 | | | |
| receptor | | | |
| domain- | | | |
| containing | | | |
| adaptor | | | |
| protein | | | |
| isoform b; | | | |
| adapter | | | |
| protein wyatt; | | | |
| MyD88 | | | |
| adapter-like | | | |
| protein; TIR | | | |
| domain- | | | |
| containing | | | |
| adapter | | | |
| protein; Toll- | | | |
| interleukin 1 | | | |
| receptor | | | |
| (TIR) | | | |
| domain- | | | |
| containing | | | |
| adaptor | | | |
| protein | | | |
| [Homo | | | |
| sapiens] | | | |
| CLDN1 | 594279 | 6.42E−03 | 0.46 |
| FLJ22471 | 668510 | 6.75E−03 | 0.56 |
| IMAGE:177857 | 177857 | 6.78E−03 | 0.74 |
| Transcribed | | | |
| locus | | | |
| IMAGE:745512 | 745512 | 6.99E−03 | 0.84 |
| SIAT8C | 382069 | 7.32E−03 | 0.83 |
| STAT1 | 110101 | 7.34E−03 | 0.81 |
| TYMS | 2242054 | 7.38E−03 | 0.83 |
| F10 | 310519 | 7.60E−03 | 0.82 |
| SLIT1 | 38403 | 7.79E−03 | 0.78 |
| IL2RA | 3054031 | 7.91E−03 | 0.81 |
| SPDEF | 1188588 | 8.08E−03 | 0.81 |
| ATP2B4 | 665814 | 8.11E−03 | 0.72 |
| TRAF4 | 667756 | 8.19E−03 | 0.82 |
| IMAGE:173818 | 173818 | 8.27E−03 | 0.81 |
| Transcribed | | | |
| locus | | | |
| IMAGE:731758 | 731758 | 8.32E−03 | 0.82 |
| Transcribed | | | |
| locus, weakly | | | |
| similar to | | | |
| NP_081070.1 | | | |
| kiaa-iso | | | |
| protein | | | |
| homolog; | | | |
| Band 47B | | | |
| [Mus | | | |
| musculus] | | | |
| CCR7 | 2345206 | 8.54E−03 | 0.85 |
| HN1 | 471568 | 8.84E−03 | 0.77 |
| ECM1 | 301122 | 8.94E−03 | 0.3 |
| C21orf84 | 730814 | 8.99E−03 | 0.75 |
| BIC | 743270 | 9.01E−03 | 0.79 |
| IMAGE:1859532 | 1859532 | 9.68E−03 | 0.81 |
| IMAGE:382423 | 382423 | 9.70E−03 | 0.73 |
| Transcribed | | | |
| locus | | | |
| PCTK3 | 725677 | 9.81E−03 | 0.8 |
| IMAGE:744410 | 744410 | 9.87E−03 | 0.82 |
| Transcribed | | | |
| locus | | | |
| SPINT1 | 723914 | 9.92E−03 | 0.83 |
| BMP5 | 1846326 | 1.00E−02 | 0.75 |
| MYC | 129438 | 1.02E−02 | 0.7 |
| TP53I11 | 667514 | 1.04E−02 | 0.63 |
| CASP3 | 823680 | 1.08E−02 | 0.75 |
| TIMP1 | 162246 | 1.10E−02 | 0.62 |
| SLC36A3 | 731115 | 1.12E−02 | 0.81 |
| CD63 | 125552 | 1.14E−02 | 0.85 |
| PTPN4 | 666367 | 1.14E−02 | 0.71 |
| AOC3 | 484535 | 1.15E−02 | 0.77 |
| IMAGE:2113771 | 2113771 | 1.20E−02 | 0.83 |
| FLJ25067 | 667252 | 1.21E−02 | 0.68 |
| PSCD3 | 744050 | 1.21E−02 | 0.83 |
| STK17A | 562904 | 1.22E−02 | 0.8 |
| HSPH1 | 666554 | 1.25E−02 | 0.79 |
| FSHPRH1 | 667355 | 1.25E−02 | 0.81 |
| GPC3 | 137131 | 1.26E−02 | 0.78 |
| MEOX1 | 760065 | 1.28E−02 | 0.78 |
| IMAGE:727289 | 727289 | 1.28E−02 | 0.71 |
| Transcribed | | | |
| locus | | | |
| IMAGE:731616 | 731616 | 1.32E−02 | 0.68 |
| MRNA; | | | |
| cDNA | | | |
| DKFZp686A102 | | | |
| (from | | | |
| clone | | | |
| DKFZp686A102) | | | |
| VTN | 230126 | 1.35E−02 | 0.74 |
| MGC45840 | 725231 | 1.43E−02 | 0.79 |
| TNIP1 | 135801 | 1.43E−02 | 0.79 |
| S100A6 | 512420 | 1.47E−02 | 0.8 |
| CALM1 | 594510 | 1.47E−02 | 0.77 |
| SDHA | 40304 | 1.50E−02 | 0.82 |
| NOTCH2 | 1641901 | 1.53E−02 | 0.64 |
| GIMAP5 | 180259 | 1.53E−02 | 0.79 |
| DIO1 | 296702 | 1.54E−02 | 0.74 |
| CHRDL2 | 485872 | 1.57E−02 | 0.85 |
| CCL28 | 136919 | 1.60E−02 | 0.83 |
| IMAGE:364741 | 364741 | 1.61E−02 | 0.74 |
| WAS | 2148946 | 1.63E−02 | 0.75 |
| PITPNC1 | 364436 | 1.66E−02 | 0.8 |
| IMAGE:136976 | 136976 | 1.72E−02 | 0.78 |
| Transcribed | | | |
| locus, | | | |
| moderately | | | |
| similar to | | | |
| NP_777603.1 | | | |
| hypothetical | | | |
| protein | | | |
| FLJ25976 | | | |
| [Homo | | | |
| sapiens] | | | |
| CD33 | 1917430 | 1.74E−02 | 0.74 |
| CLGN | 1049033 | 1.74E−02 | 0.77 |
| POPDC3 | 264166 | 1.76E−02 | 0.8 |
| RIN1 | 741406 | 1.77E−02 | 0.8 |
| GJA5 | 196338 | 1.79E−02 | 0.75 |
| UBE2L6 | 1088980 | 1.80E−02 | 0.84 |
| DNMT1 | 768241 | 1.81E−02 | 0.86 |
| IMAGE:742061 | 742061 | 1.81E−02 | 0.78 |
| BMP4 | 69166 | 1.83E−02 | 0.77 |
| SENP6 | 739237 | 1.84E−02 | 0.8 |
| COX6A1 | 512910 | 1.86E−02 | 0.78 |
| SAFB | 42280 | 1.91E−02 | 0.73 |
| PLTP | 134416 | 1.92E−02 | 0.82 |
| ME3 | 724238 | 1.96E−02 | 0.76 |
| CD47 | 357442 | 2.02E−02 | 0.83 |
| MGC23909 | 731598 | 2.05E−02 | 0.72 |
| PREB | 740347 | 2.06E−02 | 0.64 |
| EIF2C4 | 730834 | 2.09E−02 | 0.85 |
| BIN1 | 2384812 | 2.10E−02 | 0.8 |
| PML | 724554 | 2.12E−02 | 0.85 |
| PLCG2 | 201467 | 2.13E−02 | 0.78 |
| CTRL | 1308954 | 2.15E−02 | 0.83 |
| MCC | 731305 | 2.16E−02 | 0.71 |
| PANK1 | 256177 | 2.17E−02 | 0.84 |
| PRKCDBP | 157847 | 2.19E−02 | 0.79 |
| VPS26 | 687336 | 2.22E−02 | 0.85 |
| NOMO1 | 134615 | 2.24E−02 | 0.82 |
| GSTM1 | 73778 | 2.31E−02 | 0.79 |
| S100A16 | 739851 | 2.33E−02 | 0.83 |
| RPS6KA5 | 258966 | 2.33E−02 | 0.87 |
| IMAGE:136801 | 136801 | 2.40E−02 | 0.82 |
| IMAGE:383718 | 383718 | 2.40E−02 | 0.81 |
| ATP11B | 666334 | 2.43E−02 | 0.75 |
| VDAC1 | 486221 | 2.47E−02 | 0.78 |
| C15orf29 | 177775 | 2.49E−02 | 0.77 |
| PCGF4 | 740457 | 2.53E−02 | 0.78 |
| EIF4EBP1 | 713608 | 2.56E−02 | 0.83 |
| GFPT2 | 485085 | 2.60E−02 | 0.76 |
| DUSP3 | 119772 | 2.63E−02 | 0.85 |
| SASH1 | 31120 | 2.66E−02 | 0.82 |
| LCN2 | 544683 | 2.68E−02 | 0.67 |
| KLRD1 | 145696 | 2.76E−02 | 0.88 |
| IMAGE:383528 | 383528 | 2.79E−02 | 0.36 |
| Transcribed | | | |
| locus, weakly | | | |
| similar to | | | |
| XP_375099.1 | | | |
| PREDICTED: | | | |
| similar to | | | |
| hypothetical | | | |
| protein | | | |
| FLJ25224 | | | |
| [Homo | | | |
| sapiens] | | | |
| IMAGE:743619 | 743619 | 2.81E−02 | 0.81 |
| GASP5 | 341763 | 2.82E−02 | 0.71 |
| FBXO46 | 471664 | 2.82E−02 | 0.75 |
| MRC2 | 342581 | 2.84E−02 | 0.73 |
| IMAGE:726647 | 726647 | 2.87E−02 | 0.84 |
| Transcribed | | | |
| locus | | | |
| NME7 | 743982 | 2.87E−02 | 0.7 |
| IMAGE:743290 | 743290 | 2.95E−02 | 0.76 |
| Transcribed | | | |
| locus | | | |
| CD5 | 356841 | 3.06E−02 | 0.8 |
| RPL27A | 178255 | 3.06E−02 | 0.69 |
| ADAM12 | 724812 | 3.07E−02 | 0.74 |
| ICAM3 | 156183 | 3.13E−02 | 0.84 |
| GAPD | 152847 | 3.13E−02 | 0.76 |
| YWHAH | 324066 | 3.15E−02 | 0.86 |
| C6orf216 | 731742 | 3.18E−02 | 0.84 |
| SPATA5L1 | 724884 | 3.19E−02 | 0.74 |
| CITED1 | 265558 | 3.23E−02 | 0.55 |
| VIL2 | 124701 | 3.27E−02 | 0.81 |
| SLC2A4RG | 376983 | 3.35E−02 | 0.86 |
| MDH1 | 725188 | 3.38E−02 | 0.87 |
| KCNK4 | 743016 | 3.38E−02 | 0.83 |
| GSS | 140405 | 3.42E−02 | 0.82 |
| NR2F1 | 253386 | 3.48E−02 | 0.8 |
| MX1 | 713879 | 3.55E−02 | 0.82 |
| MGC5395 | 238840 | 3.57E−02 | 0.84 |
| SYP | 30471 | 3.58E−02 | 0.66 |
| RGS19 | 485803 | 3.61E−02 | 0.82 |
| CTXN1 | 179266 | 3.61E−02 | 0.86 |
| LOC284669 | 364885 | 3.61E−02 | 0.78 |
| KRT7 | 592276 | 3.63E−02 | 0.83 |
| ALDH1A3 | 486189 | 3.67E−02 | 0.76 |
| KLRK1 | 725473 | 3.71E−02 | 0.84 |
| SAP30L | 723950 | 3.81E−02 | 0.85 |
| PSEN1 | 724537 | 3.87E−02 | 0.86 |
| PDK1 | 1645668 | 3.92E−02 | 0.86 |
| MAN1A1 | 137719 | 3.93E−02 | 0.66 |
| PLCG1 | 1174287 | 4.01E−02 | 0.78 |
| IMAGE:743517 | 743517 | 4.03E−02 | 0.84 |
| Transcribed | | | |
| locus | | | |
| IMAGE:257555 | 257555 | 4.03E−02 | 0.76 |
| POU2F2 | 188393 | 4.12E−02 | 0.8 |
| IMAGE:745123 | 745123 | 4.22E−02 | 0.58 |
| Transcribed | | | |
| locus | | | |
| LILRB5 | 71428 | 4.28E−02 | 0.81 |
| LCN2 | 741497 | 4.32E−02 | 0.47 |
| USP4 | 544818 | 4.38E−02 | 0.76 |
| IMAGE:251427 | 251427 | 4.44E−02 | 0.83 |
| KIAA1280 | 366085 | 4.52E−02 | 0.85 |
| MESP1 | 25865 | 4.57E−02 | 0.86 |
| C22orf16 | 236119 | 4.63E−02 | 0.81 |
| IMAGE:485104 | 485104 | 4.72E−02 | 0.72 |
| Transcribed | | | |
| locus | | | |
| CCL1 | 1570420 | 4.72E−02 | 0.77 |
| KNS2 | 174654 | 4.79E−02 | 0.85 |
| IMAGE:745072 | 745072 | 4.82E−02 | 0.78 |
| Transcribed | | | |
| locus | | | |
| MUTED | 731202 | 4.88E−02 | 0.77 |
| KCNQ2 | 179534 | 4.90E−02 | 0.83 |
| PMS2 | 116906 | 4.92E−02 | 0.77 |
| PDCD1LG1 | 1942634 | 4.94E−02 | 0.72 |

TABLE 16

| | MNG | Over | |
|---|---|---|---|
| name | cloid | p-value | fold |
| NEXN | 687625 | 7.18E−05 | 1.40 |
| IMAGE:726782 | 726782 | 2.00E−03 | 1.43 |
| Transcribed | | | |
| locus | | | |
| PRUNE | 364324 | 2.56E−03 | 1.30 |
| GATA3 | 148796 | 2.80E−03 | 1.40 |
| IMAGE:744505 | 744505 | 2.84E−03 | 1.35 |
| IMAGE:26164 | 26164 | 3.13E−03 | 1.29 |
| TTC17 | 665668 | 3.15E−03 | 1.29 |
| FLJ20674 | 713837 | 3.84E−03 | 1.20 |
| ALCAM | 172828 | 3.93E−03 | 1.23 |
| IMAGE:731726 | 731726 | 4.48E−03 | 1.54 |
| Transcribed | | | |
| locus | | | |
| FOXO1A | 151247 | 4.60E−03 | 1.30 |
| ZNF83 | 486356 | 4.65E−03 | 1.34 |
| BCL2 | 342181 | 6.17E−03 | 1.36 |
| RODH | 471641 | 6.87E−03 | 2.44 |
| NR2F2 | 72744 | 7.02E−03 | 1.39 |
| PDLIM1 | 135689 | 8.66E−03 | 1.49 |
| C10orf116 | 740941 | 9.15E−03 | 1.67 |
| C20orf19 | 366032 | 1.03E−02 | 1.27 |
| FCGBP | 154172 | 1.26E−02 | 1.34 |
| ARL6IP2 | 743987 | 1.27E−02 | 1.26 |
| MGC17299 | 713422 | 1.28E−02 | 1.40 |
| AQP4 | 279172 | 1.28E−02 | 1.65 |
| FOLR1 | 131839 | 1.31E−02 | 1.53 |
| LOC80298 | 666551 | 1.45E−02 | 1.19 |
| IMAGE:665392 | 665392 | 1.54E−02 | 1.36 |
| Hypothetical | | | |
| LOC388790 | | | |
| LRP2 | 2055272 | 1.76E−02 | 1.44 |
| MKKS | 729957 | 1.78E−02 | 1.23 |
| NR6A1 | 258666 | 1.96E−02 | 1.29 |
| PHC3 | 743391 | 2.05E−02 | 1.12 |
| KCNC3 | 53333 | 2.15E−02 | 1.18 |
| FOXO3A | 109142 | 2.18E−02 | 1.28 |
| IMAGE:743615 | 743615 | 2.28E−02 | 1.13 |
| Full- | | | |
| length cDNA | | | |
| clone | | | |
| CS0DK001YE19 | | | |
| of HeLa | | | |
| cells Cot 25- | | | |
| normalized of | | | |
| Homo | | | |
| sapiens | | | |
| (human) | | | |
| CAV1 | 133531 | 2.29E−02 | 1.39 |
| CAV1 | 309645 | 2.31E−02 | 1.78 |
| GALT | 68972 | 2.49E−02 | 1.18 |
| IMAGE:682311 | 682311 | 2.61E−02 | 1.21 |
| LOC440722 | | | |
| IMAGE:666946 | 666946 | 2.61E−02 | 1.18 |
| FLJ21918 | 194302 | 2.73E−02 | 1.10 |
| SIN3A | 26455 | 2.80E−02 | 1.12 |
| IMAGE:744439 | 744439 | 2.86E−02 | 1.16 |
| Transcribed | | | |
| locus | | | |
| THUMPD1 | 739451 | 2.96E−02 | 1.24 |
| IL6ST | 137010 | 3.31E−02 | 1.18 |
| HDAC6 | 669295 | 3.34E−02 | 1.17 |
| KRTHA4 | 325155 | 3.60E−02 | 1.51 |
| IMAGE:731685 | 731685 | 3.71E−02 | 1.23 |
| Transcribed | | | |
| locus | | | |
| EPHB6 | 172982 | 3.75E−02 | 1.39 |
| HDAC9 | 738506 | 3.79E−02 | 1.24 |
| ESRRBL1 | 51232 | 3.82E−02 | 1.16 |
| MT1F | 78353 | 3.89E−02 | 1.39 |
| C9orf10OS | 742695 | 4.03E−02 | 1.17 |
| ETV4 | 1690788 | 4.11E−02 | 1.30 |
| LOC51760 | 52226 | 4.12E−02 | 1.27 |
| MBNL1 | 136114 | 4.19E−02 | 1.16 |
| CES2 | 153667 | 4.81E−02 | 1.14 |
| LOC286170 | 134858 | 4.81E−02 | 1.21 |
| PRKG1 | 667587 | 4.91E−02 | 1.16 |

TABLE 17

| | OTA | Under | |
|---|---|---|---|
| name | cloid | p-value | fold |
| MLL | 80688 | 1.6437E−15 | 0.51 |
| APOE | 1870594 | 9.7061E−13 | 0.39 |
| RFX2 | 731738 | 9.0218E−12 | 0.48 |
| APOE | 1870594 | 3.2318E−11 | 0.29 |
| FUS | 365348 | 5.854E−11 | 0.60 |
| IMAGE:681875 | 681875 | 6.0611E−11 | 0.70 |
| IMAGE:731751 | 731751 | 7.5532E−11 | 0.44 |
| Transcribed | | | |
| locus | | | |
| MBNL1 | 136114 | 1.6046E−10 | 0.58 |
| NPFF | 365161 | 1.7737E−10 | 0.55 |
| C4A | 491004 | 1.9841E−10 | 0.35 |
| C4A | 724366 | 3.9317E−10 | 0.33 |
| ZNF395 | 744983 | 5.8686E−10 | 0.62 |
| LOC389906 | 740718 | 6.1317E−10 | 0.72 |
| HMGB2 | 884365 | 1.3605E−09 | 0.64 |
| ANKRD28 | 687381 | 1.6567E−09 | 0.64 |
| SPINL | 236399 | 3.6199E−09 | 0.31 |
| IMAGE:731726 | 731726 | 8.8574E−09 | 0.53 |
| Transcribed | | | |
| locus | | | |
| URB | 809719 | 9.1784E−09 | 0.48 |
| CAPN3 | 757248 | 1.6524E−08 | 0.64 |
| EPHB6 | 172982 | 1.7797E−08 | 0.42 |
| IMP-2 | 743774 | 3.0771E−08 | 0.33 |
| DCN | 666410 | 3.1847E−08 | 0.51 |
| FLJ10858 | 743961 | 3.3637E−08 | 0.63 |
| G3BP | 667239 | 6.2302E−08 | 0.63 |
| GTPBP5 | 725502 | 8.9429E−08 | 0.50 |
| CDKN1B | 265345 | 1.3838E−07 | 0.65 |
| SENP6 | 366436 | 1.4497E−07 | 0.74 |
| NYREN18 | 668584 | 1.4512E−07 | 0.62 |
| NT5C2 | 725076 | 1.6229E−07 | 0.70 |
| HNRPA3 | 365349 | 1.6398E−07 | 0.65 |
| GATA3 | 148796 | 2.4888E−07 | 0.63 |
| TIAM1 | 23612 | 2.8111E−07 | 0.34 |
| IMAGE:725726 | 725726 | 2.8637E−07 | 0.68 |
| Hypothetical | | | |
| gene | | | |
| supported by | | | |
| AK093801 | | | |
| LOC286272 | 667361 | 3.1104E−07 | 0.72 |
| KCNC3 | 53333 | 3.1744E−07 | 0.60 |
| CASP7 | 279470 | 3.4531E−07 | 0.72 |
| LOC147650 | 729510 | 3.7898E−07 | 0.66 |
| SLC6A8 | 725877 | 4.4149E−07 | 0.22 |
| IMAGE:729956 | 729956 | 4.703E−07 | 0.69 |
| CXCL14 | 345034 | 5.6941E−07 | 0.11 |
| CFH | 665784 | 1.4226E−06 | 0.47 |
| SPATA12 | 730300 | 1.5763E−06 | 0.65 |
| NUP214 | 382612 | 1.6434E−06 | 0.58 |
| FLJ22794 | 137454 | 1.6888E−06 | 0.63 |
| PDLIM1 | 135689 | 1.7329E−06 | 0.59 |
| SNTB2 | 667310 | 1.8243E−06 | 0.76 |
| TLX3 | 1860115 | 1.9435E−06 | 0.53 |
| IMAGE:731714 | 731714 | 2.3202E−06 | 0.65 |
| CDNA | | | |
| FLJ26188 fis, | | | |
| clone | | | |
| ADG04821 | | | |
| IMAGE:365097 | 365097 | 2.5097E−06 | 0.75 |
| CDNA | | | |
| FLJ25129 fis, | | | |
| clone | | | |
| CBR06594 | | | |
| SNRP70 | 729971 | 2.6207E−06 | 0.57 |
| FLJ12604 | 731128 | 2.8824E−06 | 0.60 |
| IMAGE:682311 | 682311 | 2.9031E−06 | 0.71 |
| LOC440722 | | | |
| APLP2 | 549054 | 2.9384E−06 | 0.61 |
| HP1-BP74 | 726092 | 4.2033E−06 | 0.58 |
| BAZ2B | 609631 | 4.4078E−06 | 0.73 |
| PBXIP1 | 366042 | 4.4151E−06 | 0.58 |
| SEMA4B | 714437 | 4.6922E−06 | 0.70 |
| GALT | 68972 | 5.1029E−06 | 0.70 |
| SLC25A23 | 741954 | 5.2058E−06 | 0.65 |
| CICE | 725791 | 5.2466E−06 | 0.72 |
| INHBB | 730012 | 5.2829E−06 | 0.45 |
| FRMD4B | 669564 | 5.3205E−06 | 0.54 |
| MGC40214 | 666455 | 5.4593E−06 | 0.77 |
| MGC5395 | 238840 | 5.5794E−06 | 0.62 |
| CaMKIINalpha | 173820 | 6.3166E−06 | 0.63 |
| IMAGE:743485 | 743485 | 7.0609E−06 | 0.64 |
| Similar to | | | |
| RIKEN cDNA | | | |
| E130306M17 | | | |
| gene | | | |
| IMAGE:726782 | 726782 | 7.5601E−06 | 0.71 |
| Transcribed | | | |
| locus | | | |
| MRC2 | 235882 | 8.1186E−06 | 0.44 |
| MED19 | 743760 | 8.1229E−06 | 0.73 |
| PRUNE | 364324 | 8.2734E−06 | 0.67 |
| CASP3 | 823680 | 8.3059E−06 | 0.63 |
| TJP1 | 179334 | 8.8933E−06 | 0.69 |
| SEPT-02 | 729542 | 8.9128E−06 | 0.58 |
| CCBP2 | 135930 | 9.9031E−06 | 0.70 |
| HDAC9 | 738506 | 9.9758E−06 | 0.74 |
| MMP2 | 155839 | 1.0013E−05 | 0.54 |
| NFIB | 416959 | 1.043E−05 | 0.64 |
| RDH5 | 682109 | 1.0868E−05 | 0.73 |
| IMAGE:175268 | 175268 | 1.108E−05 | 0.64 |
| IMAGE:26486 | 26486 | 1.129E−05 | 0.78 |
| CDNA | | | |
| FLJ43345 fis, | | | |
| clone | | | |
| NT2RI3008228 | | | |
| RASGRP1 | 725707 | 1.1705E−05 | 0.44 |
| BCAP29 | 725970 | 1.2079E−05 | 0.56 |
| ITPR1 | 667348 | 1.2089E−05 | 0.65 |
| SGSH | 669263 | 1.2526E−05 | 0.71 |
| IMAGE:28705 | 28705 | 1.3308E−05 | 0.75 |
| CECR1 | 725612 | 1.4252E−05 | 0.74 |
| TIMP1 | 162246 | 1.4504E−05 | 0.40 |
| IMAGE:666140 | 666140 | 1.5033E−05 | 0.75 |
| Transcribed | | | |
| locus, weakly | | | |
| similar to | | | |
| XP_498467.1 | | | |
| PREDICTED: | | | |
| hypothetical | | | |
| protein | | | |
| XP_498467 | | | |
| [Homo | | | |
| sapiens] | | | |
| IMAGE:738332 | 738332 | 1.6574E−05 | 0.43 |
| Transcribed | | | |
| locus | | | |
| SIAT4B | 365877 | 1.7795E−05 | 0.78 |
| C10orf116 | 740941 | 1.8397E−05 | 0.49 |
| SYN3 | 727056 | 1.8892E−05 | 0.52 |
| APOD | 838611 | 1.9338E−05 | 0.30 |
| LOC286076 | 731275 | 1.9611E−05 | 0.64 |
| F10 | 310519 | 2.0346E−05 | 0.73 |
| IMAGE:384087 | 384087 | 2.1119E−05 | 0.69 |
| STYX | 174879 | 2.2844E−05 | 0.68 |
| PLA2G6 | 744087 | 2.6403E−05 | 0.60 |
| IMAGE:727496 | 727496 | 2.6792E−05 | 0.53 |
| Transcribed | | | |
| locus | | | |
| IMAGE:379279 | 379279 | 2.7991E−05 | 0.76 |
| COG3 | 25226 | 2.8031E−05 | 0.70 |
| IMAGE:731428 | 731428 | 3.4732E−05 | 0.75 |
| Transcribed | | | |
| locus, weakly | | | |
| similar to | | | |
| XP_521389.1 | | | |
| PREDICTED: | | | |
| similar to | | | |
| UTY [Pan troglodytes] | | | |
| IMAGE:365990 | 365990 | 3.8574E−05 | 0.69 |
| FMNL2 | 364568 | 4.0777E−05 | 0.76 |
| IMAGE:2266583 | 2266583 | 4.0793E−05 | 0.69 |
| MDGA1 | 180082 | 4.196E−05 | 0.73 |
| MGP | 590264 | 4.9648E−05 | 0.53 |
| LRP5 | 725836 | 5.9288E−05 | 0.72 |
| NFYA | 731648 | 5.9861E−05 | 0.75 |
| NR2F1 | 253386 | 6.0806E−05 | 0.71 |
| FOXO1A | 151247 | 6.436E−05 | 0.65 |
| PPARD | 724562 | 6.7271E−05 | 0.71 |
| FLJ20674 | 713837 | 6.9459E−05 | 0.81 |
| COL27A1 | 379517 | 6.9891E−05 | 0.71 |
| SALL4 | 726454 | 7.059E−05 | 0.37 |
| AXIN2 | 135887 | 7.4334E−05 | 0.70 |
| LOC126669 | 682088 | 7.448E−05 | 0.70 |
| TETRAN | 725340 | 8.5261E−05 | 0.73 |
| CTH | 730606 | 8.5368E−05 | 0.72 |
| CCL5 | 840753 | 8.8355E−05 | 0.68 |
| PPP1CA | 1422791 | 9.3449E−05 | 0.72 |
| IMAGE:723752 | 723752 | 9.349E−05 | 0.72 |
| PRKCQ | 669149 | 9.6294E−05 | 0.74 |
| RGL2 | 741891 | 0.00010227 | 0.75 |
| HDAC6 | 669295 | 0.00010356 | 0.75 |
| BTG1 | 382760 | 0.0001116 | 0.74 |
| ABCC3 | 208097 | 0.00011391 | 0.50 |
| APOL1 | 665632 | 0.00011456 | 0.48 |
| IMAGE:731665 | 731665 | 0.00012147 | 0.70 |
| FARP1 | 486708 | 0.0001285 | 0.68 |
| SFRP4 | 285693 | 0.00013182 | 0.49 |
| IL19 | 1932168 | 0.00013629 | 0.59 |
| IQCF3 | 726578 | 0.00014161 | 0.73 |
| HDAC1 | 1896337 | 0.00014481 | 0.71 |
| UBE1L | 250883 | 0.00014933 | 0.63 |
| FLJ34433 | 713671 | 0.00015152 | 0.77 |
| SMARCA5 | 730037 | 0.00015195 | 0.75 |
| TPM2 | 740620 | 0.0001529 | 0.59 |
| UPF2 | 682149 | 0.0001531 | 0.79 |
| PMS2 | 116906 | 0.00016313 | 0.59 |
| IMAGE:669470 | 669470 | 0.0001635 | 0.80 |
| CDNA | | | |
| clone | | | |
| IMAGE:5206119, | | | |
| partial | | | |
| cds | | | |
| GCLC | 1526058 | 0.0001647 | 0.78 |
| CITED2 | 365090 | 0.00016528 | 0.60 |
| GRK5 | 740245 | 0.00016593 | 0.71 |
| ETV1 | 24541 | 0.00016784 | 0.76 |
| NCK1 | 1326169 | 0.00017218 | 0.66 |
| CRYZ | 486935 | 0.00018293 | 0.83 |
| SPTLC2 | 365000 | 0.00019182 | 0.78 |
| IMAGE:727123 | 727123 | 0.00020299 | 0.77 |
| Transcribed | | | |
| locus, | | | |
| strongly | | | |
| similar to | | | |
| NP_004491.1 | | | |
| heterogeneous | | | |
| nuclear | | | |
| ribonucleoprotein C | | | |
| isoform b; | | | |
| nuclear | | | |
| ribonucleoprotein | | | |
| particle | | | |
| C2 protein; | | | |
| nuclear | | | |
| ribonucleoprotein | | | |
| particle | | | |
| C1 protein | | | |
| [Homo | | | |
| sapiens] | | | |
| IMAGE:1877668 | 1877668 | 0.00020813 | 0.74 |
| PTPRS | 666066 | 0.00021386 | 0.74 |
| IRAK1BP1 | 687306 | 0.0002186 | 0.75 |
| NBL1 | 503874 | 0.00022339 | 0.50 |
| PSPC1 | 687541 | 0.00023946 | 0.76 |
| PDCD1LG2 | 738944 | 0.00025058 | 0.71 |
| C9orf72 | 726849 | 0.0002553 | 0.70 |
| CA3 | 365609 | 0.0002601 | 0.74 |
| DNALI1 | 782688 | 0.0002748 | 0.62 |
| PIAS1 | 38789 | 0.00028384 | 0.73 |
| GLT8D2 | 365271 | 0.00028457 | 0.57 |
| CYP51A1 | 739901 | 0.00030959 | 0.73 |
| IMAGE:145112 | 145112 | 0.00031549 | 0.71 |
| CDKN1C | 2381770 | 0.00031705 | 0.69 |
| IMAGE:742853 | 742853 | 0.00032014 | 0.50 |
| AOC3 | 484535 | 0.00032283 | 0.69 |
| CD5 | 356841 | 0.00032358 | 0.71 |
| PHC3 | 743391 | 0.00033419 | 0.79 |
| GLG1 | 365613 | 0.00034049 | 0.70 |
| DJ462O23.2 | 738970 | 0.00034686 | 0.74 |
| FLJ35794 | 364204 | 0.00035268 | 0.64 |
| PREB | 740347 | 0.00037117 | 0.45 |
| TFDP2 | 742806 | 0.00038692 | 0.66 |
| CAPNS1 | 152655 | 0.00038965 | 0.78 |
| LILRB4 | 2341829 | 0.00042057 | 0.77 |
| SLC9A5 | 112819 | 0.00042518 | 0.66 |
| LASP1 | 592598 | 0.00043231 | 0.42 |
| ZNF580 | 668089 | 0.00043314 | 0.74 |
| FGFRL1 | 739956 | 0.00043483 | 0.64 |
| ERK8 | 714049 | 0.00043612 | 0.72 |
| IMAGE:668684 | 668684 | 0.00044757 | 0.76 |
| Transcribed | | | |
| locus, weakly | | | |
| similar to | | | |
| XP_209041.2 | | | |
| PREDICTED: | | | |
| similar to | | | |
| KIAA1503 | | | |
| protein | | | |
| [Homo | | | |
| sapiens] | | | |
| GLMN | 293916 | 0.00044948 | 0.84 |
| DF | 666128 | 0.00045614 | 0.49 |
| PDGFRL | 139242 | 0.00045735 | 0.61 |
| ESRRBL1 | 51232 | 0.00046822 | 0.70 |
| DKFZp434M202 | 743118 | 0.00047312 | 0.73 |
| KLRD1 | 2728204 | 0.00051707 | 0.67 |
| HMGN1 | 565754 | 0.00051902 | 0.72 |
| MAP3K4 | 727229 | 0.00053005 | 0.76 |
| IMAGE:2254555 | 2254555 | 0.00056693 | 0.73 |
| THEM2 | 742543 | 0.000571 | 0.68 |
| IKBKE | 364448 | 0.00058383 | 0.78 |
| FLJ20254 | 135096 | 0.00060077 | 0.75 |
| ZNF251 | 486401 | 0.00060459 | 0.34 |
| FLJ10707 | 364575 | 0.00063587 | 0.79 |
| AQP11 | 713831 | 0.00064759 | 0.64 |
| ARHGEF6 | 687990 | 0.00068864 | 0.80 |
| IMAGE:668924 | 668924 | 0.00072815 | 0.79 |
| IMAGE:738896 | 738896 | 0.00075859 | 0.29 |
| FOLR1 | 131839 | 0.00076028 | 0.60 |
| SULT1E1 | 207926 | 0.00078718 | 0.80 |
| HIS1 | 342551 | 0.00080399 | 0.72 |
| SEMA4D | 210587 | 0.00080847 | 0.76 |
| CCND1 | 324079 | 0.00085252 | 0.67 |
| PSAT1 | 366388 | 0.00087487 | 0.66 |
| CCNL1 | 251216 | 0.00087862 | 0.68 |
| MGC3329 | 137005 | 0.00088837 | 0.77 |
| C10orf61 | 741894 | 0.00095106 | 0.72 |
| LIPT1 | 22085 | 0.00095361 | 0.71 |
| VCL | 44193 | 0.00103155 | 0.79 |
| FOXO3A | 109142 | 0.0010426 | 0.75 |
| PGR1 | 666361 | 0.00108576 | 0.76 |
| CREB1 | 148212 | 0.00109064 | 0.81 |
| GSS | 140405 | 0.00109778 | 0.74 |
| SSH3 | 726272 | 0.00114183 | 0.75 |
| SEPT-05 | 448163 | 0.00117115 | 0.26 |
| MORF4L1 | 135875 | 0.00122685 | 0.75 |
| BAT3 | 24392 | 0.00123243 | 0.75 |
| IMAGE:725401 | 725401 | 0.00124171 | 0.77 |
| SF3B1 | 739247 | 0.0012449 | 0.67 |
| APOL3 | 366289 | 0.00126395 | 0.64 |
| S100A4 | 868577 | 0.00130679 | 0.44 |
| LOC286170 | 134858 | 0.00131926 | 0.69 |
| COL1A1 | 140347 | 0.00137465 | 0.51 |
| DNCLI2 | 261656 | 0.00138781 | 0.74 |
| PDCD4 | 665376 | 0.00144209 | 0.59 |
| FLJ27523 | 743246 | 0.00146851 | 0.45 |
| PLXNB2 | 1420676 | 0.00149466 | 0.69 |
| ZNF512 | 726596 | 0.00156733 | 0.72 |
| CST3 | 357201 | 0.00160531 | 0.75 |
| WHSC2 | 668283 | 0.00162671 | 0.75 |
| ALCAM | 172828 | 0.00163738 | 0.77 |
| NOG | 487474 | 0.00165986 | 0.73 |
| IMAGE:665833 | 665833 | 0.00178979 | 0.77 |
| CAV1 | 309645 | 0.00182486 | 0.59 |
| MTUS1 | 376597 | 0.00182533 | 0.52 |
| IMAGE:731685 | 731685 | 0.0018723 | 0.83 |
| Transcribed | | | |
| locus | | | |
| IKBKB | 2484742 | 0.00188154 | 0.81 |
| PP784 | 366089 | 0.00188347 | 0.74 |
| CITED2 | 286138 | 0.00188602 | 0.76 |
| TYMS | 687912 | 0.00193104 | 0.73 |
| CD3E | 1536968 | 0.00197216 | 0.70 |
| USP13 | 666007 | 0.00198696 | 0.74 |
| RIPK3 | 667313 | 0.00202685 | 0.68 |
| NOTCH2 | 1641901 | 0.00206595 | 0.54 |
| TNFSF12 | 136361 | 0.00207861 | 0.82 |
| CA11 | 282587 | 0.0020984 | 0.74 |
| WDR20 | 665156 | 0.00234165 | 0.83 |
| ZNF398 | 365311 | 0.00235137 | 0.72 |
| FLJ12903 | 730123 | 0.00243509 | 0.82 |
| EVI5L | 173119 | 0.00249305 | 0.72 |
| NCAM1 | 366842 | 0.00252012 | 0.74 |
| GALNT9 | 178750 | 0.00257206 | 0.77 |
| CCL19 | 1707527 | 0.00264302 | 0.17 |
| SATB1 | 364510 | 0.00277604 | 0.79 |
| DHRS6 | 364412 | 0.00279084 | 0.76 |
| RNF123 | 366159 | 0.00283529 | 0.79 |
| IMAGE:668707 | 668707 | 0.00284093 | 0.76 |
| TGF- | | | |
| betaIIR beta | | | |
| CRLF3 | 279150 | 0.00284281 | 0.78 |
| THUMPD1 | 739451 | 0.00284724 | 0.77 |
| DGKD | 365408 | 0.00291163 | 0.78 |
| TSC | 745490 | 0.00296358 | 0.52 |
| EED | 667365 | 0.00305049 | 0.76 |
| GRB7 | 510318 | 0.00305513 | 0.83 |
| PIK3CA | 2238108 | 0.00307726 | 0.44 |
| CCNE1 | 68950 | 0.00312811 | 0.71 |
| TIE1 | 743043 | 0.00338928 | 0.73 |
| STAT2 | 2306096 | 0.00346577 | 0.79 |
| POLR2A | 740130 | 0.00347566 | 0.73 |
| AMPD2 | 669141 | 0.00356607 | 0.78 |
| IMAGE:743722 | 743722 | 0.00368853 | 0.76 |
| IMAGE:22384 | 22384 | 0.00374794 | 0.80 |
| CDNA | | | |
| FLJ34183 fis, | | | |
| clone | | | |
| FCBBF3016987 | | | |
| IGF1R | 682555 | 0.00381952 | 0.80 |
| RANBP10 | 682251 | 0.0038256 | 0.80 |
| ZNF651 | 669181 | 0.00385349 | 0.83 |
| TPD52 | 743259 | 0.00385608 | 0.28 |
| KLF1 | 208991 | 0.00386372 | 0.66 |
| IMAGE:342256 | 342256 | 0.00392068 | 0.32 |
| KIAA1838 | 744106 | 0.00395933 | 0.79 |
| TOP1 | 666425 | 0.0039984 | 0.83 |
| HOXB6 | 738358 | 0.00426232 | 0.81 |
| LAPTM4A | 726684 | 0.00435163 | 0.66 |
| IMAGE:365562 | 365562 | 0.00436529 | 0.78 |
| Transcribed | | | |
| locus, | | | |
| moderately | | | |
| similar to | | | |
| XP_521389.1 | | | |
| PREDICTED: | | | |
| similar to | | | |
| UTY [Pan | | | |
| troglodytes] | | | |
| ANKRD20B | 745106 | 0.00437688 | 0.66 |
| IMAGE:665392 | 665392 | 0.00443685 | 0.79 |
| Hypothetical | | | |
| LOC388790 | | | |
| MGAT3 | 731060 | 0.00445388 | 0.78 |
| NEXN | 687625 | 0.00446474 | 0.81 |
| DJ159A19.3 | 23945 | 0.0044943 | 0.75 |
| SELE | 1239535 | 0.004537 | 0.78 |
| ATXN1 | 682528 | 0.00455958 | 0.75 |
| LOC440712 | 725268 | 0.00473002 | 0.71 |
| GPR124 | 486493 | 0.00477135 | 0.79 |
| DAXX | 292042 | 0.00488144 | 0.83 |
| ZBTB16 | 2467442 | 0.00498586 | 0.67 |
| DNCLI2 | 134671 | 0.00499696 | 0.74 |
| ZNF83 | 486356 | 0.00506848 | 0.76 |
| DELGEF | 665437 | 0.00509526 | 0.78 |
| IFITM3 | 713623 | 0.00515614 | 0.76 |
| IMAGE:666029 | 666029 | 0.00516625 | 0.71 |
| CDNA | | | |
| FLJ43311 fis, | | | |
| clone | | | |
| NT2RI2009855 | | | |
| C19orf33 | 379540 | 0.0053682 | 0.69 |
| PDCD1LG1 | 1942634 | 0.00541203 | 0.79 |
| FLJ12592 | 252489 | 0.0054876 | 0.77 |
| RAD51L1 | 295412 | 0.00558334 | 0.69 |
| EMX2 | 365121 | 0.00561885 | 0.73 |
| CLDN1 | 594279 | 0.00570286 | 0.44 |
| HSPB1 | 23827 | 0.00579753 | 0.66 |
| C21orf55 | 666517 | 0.00584797 | 0.76 |
| TBC1D22A | 682479 | 0.00589263 | 0.81 |
| CNN1 | 726779 | 0.0058977 | 0.76 |
| DKFZp761P0423 | 730829 | 0.00596086 | 0.67 |
| IMAGE:21808 | 21808 | 0.00612512 | 0.80 |
| Transcribed | | | |
| locus | | | |
| YTHDF3 | 135450 | 0.00614088 | 0.76 |
| LGALS3BP | 742100 | 0.00646127 | 0.58 |
| LOC388886 | 34007 | 0.00661606 | 0.76 |
| SLAMF8 | 288807 | 0.00672479 | 0.73 |
| DEK | 1016390 | 0.00680353 | 0.82 |
| PDK2 | 114008 | 0.00684106 | 0.85 |
| GLG1 | 366254 | 0.00690562 | 0.83 |
| MGC17299 | 713422 | 0.00716637 | 0.83 |
| TCEA2 | 730149 | 0.00727116 | 0.83 |
| HBB | 469549 | 0.00736441 | 0.52 |
| FLJ31438 | 135539 | 0.00740367 | 0.81 |
| HCLS1 | 665452 | 0.00742076 | 0.73 |
| CLPX | 725394 | 0.00742676 | 0.81 |
| PLEKHH2 | 665254 | 0.00803369 | 0.73 |
| MMRN1 | 759865 | 0.00838721 | 0.78 |
| LOC400451 | 667174 | 0.0084152 | 0.73 |
| MGC4707 | 744945 | 0.00868037 | 0.83 |
| BBS4 | 726634 | 0.00906801 | 0.83 |
| LOC283070 | 172785 | 0.00927382 | 0.74 |
| MAPK14 | 2011082 | 0.0094865 | 0.79 |
| MXD3 | 714013 | 0.00950378 | 0.82 |
| KIAA0789 | 33621 | 0.00966515 | 0.66 |
| NOTCH1 | 359461 | 0.0098421 | 0.79 |
| GPC3 | 137131 | 0.01032142 | 0.78 |
| KCNK5 | 134978 | 0.01043433 | 0.70 |
| ECM1 | 301122 | 0.01051771 | 0.29 |
| FLJ12895 | 23271 | 0.01069841 | 0.76 |
| PLAC9 | 366115 | 0.01071461 | 0.68 |
| LOH12CR2 | 366074 | 0.01111041 | 0.76 |
| MEF2C | 687270 | 0.01126036 | 0.86 |
| IMAGE:687667 | 687667 | 0.0113645 | 0.56 |
| CDNA | | | |
| clone | | | |
| IMAGE:4811759, | | | |
| partial | | | |
| cds | | | |
| VEGFC | 503189 | 0.01190861 | 0.79 |
| CCNE1 | 357807 | 0.01195592 | 0.78 |
| IMAGE:743415 | 743415 | 0.01197603 | 0.82 |
| Transcribed | | | |
| locus | | | |
| OAZ2 | 128694 | 0.01241057 | 0.84 |
| IMAGE:731398 | 731398 | 0.01252095 | 0.80 |
| GIMAP5 | 180259 | 0.01300622 | 0.76 |
| CUGBP2 | 666235 | 0.01304288 | 0.84 |
| IMAGE:667353 | 667353 | 0.01314807 | 0.83 |
| Transcribed | | | |
| locus | | | |
| IGF1R | 682555 | 0.0132764 | 0.83 |
| ACTR1B | 136868 | 0.0134187 | 0.84 |
| BZRP | 135991 | 0.01347669 | 0.79 |
| KIAA1958 | 687287 | 0.01353299 | 0.79 |
| MAPK14 | 1741589 | 0.01358144 | 0.80 |
| TOE1 | 52897 | 0.01361943 | 0.81 |
| KIAA0515 | 687852 | 0.0136789 | 0.79 |
| GATA4 | 781738 | 0.01434771 | 0.79 |
| IMAGE:26164 | 26164 | 0.01438992 | 0.83 |
| PLA2G2A | 152802 | 0.01468617 | 0.71 |
| IMAGE:1089025 | 1089025 | 0.01469019 | 0.52 |
| FCGBP | 154172 | 0.01497258 | 0.69 |
| PTK2B | 43541 | 0.0150726 | 0.86 |
| GNB1 | 24300 | 0.01516968 | 0.78 |
| GPR108 | 25949 | 0.01526513 | 0.83 |
| PCTK3 | 725677 | 0.01530815 | 0.78 |
| SMAD4 | 214611 | 0.01537935 | 0.83 |
| IMAGE:744439 | 744439 | 0.01590113 | 0.86 |
| Transcribed | | | |
| locus | | | |
| IMAGE:23872 | 23872 | 0.01600529 | 0.82 |
| AF034176 | | | |
| Human | | | |
| mRNA | | | |
| (Tripodis and | | | |
| Ragoussis) | | | |
| Homo | | | |
| sapiens | | | |
| cDNA clone | | | |
| ntcon5 contig | | | |
| LILRA1 | 2032639 | 0.01630429 | 0.71 |
| PH-4 | 730938 | 0.01635938 | 0.85 |
| PET112L | 743125 | 0.01665049 | 0.76 |
| PTHLH | 2214396 | 0.01725027 | 0.69 |
| CD9 | 727251 | 0.01759599 | 0.82 |
| SPON2 | 723923 | 0.01769163 | 0.78 |
| KIAA1196 | 738938 | 0.01786927 | 0.80 |
| SMAD4 | 134785 | 0.01807119 | 0.82 |
| BTBD14B | 173288 | 0.01820373 | 0.76 |
| PVRL2 | 725364 | 0.01827273 | 0.82 |
| SIN3A | 26455 | 0.01860378 | 0.84 |
| CXCL12 | 213113 | 0.01879575 | 0.84 |
| MXD4 | 730036 | 0.01889125 | 0.77 |
| IMAGE:687896 | 687896 | 0.01910117 | 0.84 |
| PREDICTED: | | | |
| Homo | | | |
| sapiens | | | |
| olfactory | | | |
| receptor, | | | |
| family 7, | | | |
| subfamily E, | | | |
| member 31 | | | |
| pseudogene | | | |
| (OR7E31P), | | | |
| mRNA | | | |
| PRKG1 | 667587 | 0.01917332 | 0.83 |
| IMAGE:22908 | 22908 | 0.01931064 | 0.73 |
| Transcribed | | | |
| locus, weakly | | | |
| similar to | | | |
| NP_775735.1 | | | |
| I(3)mbt-like | | | |
| 4 [Homo | | | |
| sapiens] | | | |
| LOC340073 | 731404 | 0.01951017 | 0.73 |
| KRTHA4 | 325155 | 0.01952696 | 0.67 |
| C14orf140 | 731423 | 0.0201277 | 0.86 |
| IMAGE:46991 | 46991 | 0.020745 | 0.75 |
| Similar to | | | |
| breakpoint | | | |
| cluster region | | | |
| isoform 1 | | | |
| RBBP7 | 727457 | 0.02094568 | 0.85 |
| BCL2L11 | 300194 | 0.02100048 | 0.81 |
| IMAGE:669136 | 669136 | 0.02136336 | 0.87 |
| Transcribed | | | |
| locus, weakly | | | |
| similar to | | | |
| NP_060312.1 | | | |
| hypothetical | | | |
| protein | | | |
| FLJ20489 | | | |
| [Homo | | | |
| sapiens] | | | |
| CTNNA1 | 268972 | 0.02150881 | 0.83 |
| IMAGE:726513 | 726513 | 0.02197021 | 0.82 |
| Similar to | | | |
| 40S | | | |
| ribosomal | | | |
| protein S3 | | | |
| PSIP1 | 667598 | 0.02215201 | 0.78 |
| CAV1 | 133531 | 0.02220907 | 0.76 |
| MESDC2 | 713653 | 0.02242909 | 0.82 |
| LRP1 | 30219 | 0.02320607 | 0.74 |
| KLF4 | 188232 | 0.02332609 | 0.72 |
| FAM36A | 727263 | 0.02337422 | 0.75 |
| IMAGE:53081 | 53081 | 0.02360138 | 0.77 |
| ACTB | 203166 | 0.02394687 | 0.84 |
| EPHA4 | 2237263 | 0.02437397 | 0.77 |
| ISG20 | 740604 | 0.02440903 | 0.77 |
| ARL6IP2 | 743987 | 0.0246722 | 0.82 |
| IMAGE:738945 | 738945 | 0.02503128 | 0.86 |
| IQCA | 669510 | 0.02514619 | 0.78 |
| IFNAR2 | 123950 | 0.0253019 | 0.80 |
| CDC42EP2 | 26651 | 0.02564027 | 0.83 |
| IMAGE:665342 | 665342 | 0.02624304 | 0.85 |
| Transcribed | | | |
| locus, | | | |
| strongly | | | |
| similar to | | | |
| NP_065847.1 | | | |
| semaphorin | | | |
| 6A1; | | | |
| semaphorin | | | |
| 6A-1 [Homo | | | |
| sapiens] | | | |
| PTK2 | 724892 | 0.02711896 | 0.86 |
| NFKB1 | 687782 | 0.02773524 | 0.79 |
| FLJ21918 | 194302 | 0.02783552 | 0.85 |
| RANBP10 | 307025 | 0.02789145 | 0.85 |
| IMAGE:727154 | 727154 | 0.02978266 | 0.73 |
| Transcribed | | | |
| locus | | | |
| CAV1 | 1911930 | 0.03051702 | 0.69 |
| HBA2 | 469647 | 0.0305271 | 0.58 |
| RODH | 471641 | 0.03080742 | 0.59 |
| C20orf19 | 366032 | 0.03094876 | 0.86 |
| NFE2L2 | 29541 | 0.03104685 | 0.81 |
| DALRD3 | 740224 | 0.03140547 | 0.74 |
| HSPB1 | 724150 | 0.03142051 | 0.73 |
| IMAGE:136598 | 136598 | 0.03145463 | 0.84 |
| MRNA; | | | |
| cDNA | | | |
| DKFZp686D22106 | | | |
| (from | | | |
| clone | | | |
| DKFZp686D22106) | | | |
| CES2 | 153667 | 0.03153081 | 0.85 |
| ABCC5 | 212366 | 0.03156946 | 0.83 |
| LATS2 | 666778 | 0.03163588 | 0.78 |
| FMO5 | 364526 | 0.03165625 | 0.78 |
| IMAGE:738966 | 738966 | 0.03174724 | 0.71 |
| Transcribed | | | |
| locus | | | |
| S100A2 | 3659591 | 0.03190236 | 0.83 |
| RPS6KA2 | 22711 | 0.0320225 | 0.79 |
| FABP5 | 1088781 | 0.03220196 | 0.56 |
| IMAGE:744505 | 744505 | 0.03276021 | 0.80 |
| ZFOC1 | 744606 | 0.03369331 | 0.83 |
| IMAGE:135203 | 135203 | 0.03392561 | 0.86 |
| Transcribed | | | |
| locus | | | |
| ADCY3 | 667061 | 0.03422424 | 0.87 |
| FLJ32731 | 365177 | 0.03436124 | 0.81 |
| HIVEP1 | 758037 | 0.03478063 | 0.88 |
| ASGR1 | 25883 | 0.03536376 | 0.73 |
| TM4SF8 | 713647 | 0.03671912 | 0.82 |
| MGC10561 | 740580 | 0.03741305 | 0.84 |
| LOC146909 | 726353 | 0.0379498 | 0.82 |
| FBXL16 | 178908 | 0.03911145 | 0.84 |
| RNASE4 | 156720 | 0.03921697 | 0.84 |
| CABIN1 | 1844968 | 0.04247472 | 0.80 |
| IL6ST | 137010 | 0.04386624 | 0.82 |
| IMAGE:136801 | 136801 | 0.04568167 | 0.85 |
| CHRAC1 | 724276 | 0.04626673 | 0.82 |
| RGS6 | 24176 | 0.04643662 | 0.82 |
| TUSC1 | 740381 | 0.04785098 | 0.84 |
| POL1 | 137720 | 0.04797886 | 0.78 |
| KRT18 | 725096 | 0.04854209 | 0.80 |
| TPCN1 | 179288 | 0.04857424 | 0.86 |
| TTC17 | 665668 | 0.04953371 | 0.81 |

TABLE 18

| | OTA | Over | |
|---|---|---|---|
| name | cloid | p-value | fold |
| SERPINB5 | 1662274 | 1.34E−05 | 1.70 |
| KLRD1 | 145696 | 2.28E−05 | 1.61 |
| OBSCN | 730926 | 4.16E−05 | 1.66 |
| SLC36A3 | 731115 | 4.18E−05 | 1.60 |
| IGKV1-5 | 155345 | 4.47E−05 | 1.48 |
| ST5 | 376356 | 1.05E−04 | 1.30 |
| IMAGE:666794 | 666794 | 0.00013 | 1.89 |
| Hypothetical | | | |
| LOC388170 | | | |
| AP2S1 | 739109 | 1.54E−04 | 1.34 |
| KCNQ2 | 179534 | 1.72E−04 | 2.09 |
| GPD1 | 628418 | 1.96E−04 | 1.52 |
| RIN1 | 741406 | 2.48E−04 | 1.86 |
| CHRDL2 | 485872 | 2.57E−04 | 1.73 |
| DCAMKL1 | 232388 | 2.72E−04 | 1.60 |
| SIAT8F | 667110 | 2.77E−04 | 2.03 |
| DC12 | 724895 | 2.88E−04 | 1.92 |
| SIAT8B | 33133 | 3.13E−04 | 1.60 |
| EIF2C4 | 730834 | 3.16E−04 | 1.49 |
| BIC | 743270 | 3.28E−04 | 1.81 |
| ARHGAP10 | 731380 | 3.60E−04 | 1.99 |
| LEF1 | 713913 | 3.70E−04 | 1.71 |
| NTRK2 | 2048801 | 4.50E−04 | 2.29 |
| IMAGE:485104 | 485104 | 4.70E−04 | 2.48 |
| Transcribed | | | |
| locus | | | |
| RNF32 | 731422 | 4.98E−04 | 2.09 |
| SQLE | 124781 | 5.03E−04 | 1.66 |
| IMAGE:731616 | 731616 | 5.26E−04 | 3.04 |
| MRNA; | | | |
| cDNA | | | |
| DKFZp686A102 | | | |
| (from | | | |
| clone | | | |
| DKFZp686A102) | | | |
| SCMH1 | 724014 | 6.07E−04 | 2.30 |
| DEXI | 668186 | 6.32E−04 | 1.36 |
| MGC23909 | 731598 | 6.41E−04 | 2.10 |
| BAP1 | 1012990 | 7.10E−04 | 1.80 |
| ANP32E | 382738 | 7.26E−04 | 2.05 |
| TRIM8 | 714498 | 7.33E−04 | 1.56 |
| IMAGE:745072 | 745072 | 7.40E−04 | 1.93 |
| Transcribed | | | |
| locus | | | |
| PDCD1LG1 | 1942634 | 7.59E−04 | 1.52 |
| MRC2 | 342581 | 7.65E−04 | 2.22 |
| MAP2 | 347503 | 7.82E−04 | 1.76 |
| C6orf216 | 731742 | 7.88E−04 | 1.57 |
| PSEN1 | 724537 | 7.95E−04 | 1.31 |
| HSPC159 | 365045 | 0.00081 | 1.76 |
| IMAGE:738424 | 738424 | 8.19E−04 | 1.34 |
| IMAGE:724556 | 724556 | 8.66E−04 | 1.50 |
| Transcribed | | | |
| locus | | | |
| C21orf84 | 730814 | 8.68E−04 | 2.00 |
| OAS1 | 666703 | 9.26E−04 | 2.23 |
| CASP5 | 341763 | 9.75E−04 | 2.21 |
| S100A16 | 739851 | 1.01E−03 | 1.47 |
| IMAGE:744841 | 744841 | 1.03E−03 | 1.41 |
| LOC441054 | | | |
| ULK4 | 744895 | 1.08E−03 | 1.64 |
| FAM44C | 726894 | 1.12E−03 | 1.33 |
| COX6A1 | 512910 | 1.14E−03 | 1.51 |
| FLJ11184 | 384404 | 1.18E−03 | 1.80 |
| DAB1 | 382621 | 1.21E−03 | 1.54 |
| IMAGE:744074 | 744074 | 1.27E−03 | 2.29 |
| Transcribed | | | |
| locus | | | |
| TIMP2 | 258127 | 1.32E−03 | 1.39 |
| IMAGE:364741 | 364741 | 1.34E−03 | 2.17 |
| PSG3 | 136747 | 1.35E−03 | 1.64 |
| PRKCABP | 1174342 | 1.35E−03 | 1.40 |
| SLC6A1 | 177967 | 1.37E−03 | 1.86 |
| MEOX1 | 760065 | 1.49E−03 | 1.93 |
| IMAGE:177857 | 177857 | 1.53E−03 | 2.02 |
| Transcribed | | | |
| locus | | | |
| MGC19604 | 687468 | 1.55E−03 | 1.36 |
| SAP30L | 723950 | 1.58E−03 | 1.41 |
| BIN1 | 2384812 | 1.64E−03 | 1.53 |
| PTGES | 504646 | 1.64E−03 | 1.43 |
| IMAGE:740105 | 740105 | 1.64E−03 | 1.53 |
| KIAA1754L | 365056 | 0.00167 | 1.75 |
| LOC56251 | 137478 | 1.68E−03 | 1.57 |
| IMAGE:378458 | 378458 | 1.69E−03 | 1.55 |
| GNAI1 | 753215 | 1.73E−03 | 1.44 |
| PARD3 | 724642 | 1.75E−03 | 1.57 |
| AURKC | 731021 | 1.76E−03 | 1.69 |
| IL2RA | 3054031 | 1.76E−03 | 1.69 |
| RPL27A | 178255 | 1.79E−03 | 3.05 |
| F5 | 433155 | 1.87E−03 | 1.65 |
| FOXN4 | 731076 | 1.90E−03 | 1.85 |
| IMAGE:744410 | 744410 | 1.92E−03 | 1.56 |
| Transcribed | | | |
| locus | | | |
| IMAGE:743603 | 743603 | 1.93E−03 | 1.66 |
| LOC388284 | 731722 | 1.97E−03 | 1.47 |
| LOC440434 | 742046 | 1.99E−03 | 1.34 |
| IMAGE:727289 | 727289 | 2.04E−03 | 1.77 |
| Transcribed | | | |
| locus | | | |
| SRGAP2 | 714493 | 2.13E−03 | 1.65 |
| SYNPO | 178792 | 2.14E−03 | 1.40 |
| VAV2 | 1581686 | 2.16E−03 | 1.32 |
| S100A12 | 1705397 | 2.17E−03 | 1.46 |
| CTRL | 1308954 | 2.29E−03 | 1.39 |
| C2orf7 | 366243 | 2.30E−03 | 1.28 |
| FLJ25067 | 667252 | 2.36E−03 | 2.56 |
| PPP2R4 | 731590 | 2.38E−03 | 1.43 |
| ME3 | 724238 | 2.43E−03 | 2.15 |
| IMAGE:730924 | 730924 | 2.50E−03 | 1.36 |
| Transcribed | | | |
| locus | | | |
| SLC39A2 | 504596 | 2.62E−03 | 1.38 |
| USP4 | 544818 | 2.64E−03 | 1.80 |
| JUP | 35628 | 2.70E−03 | 1.96 |
| VDAC1 | 486221 | 2.72E−03 | 1.46 |
| TM4SF9 | 381032 | 2.75E−03 | 1.30 |
| KLHL9 | 501527 | 2.76E−03 | 1.31 |
| CLECSF12 | 258865 | 2.77E−03 | 3.51 |
| GPI | 741474 | 2.79E−03 | 1.35 |
| SPATA5L1 | 724884 | 2.81E−03 | 1.72 |
| IGHM | 276658 | 2.82E−03 | 1.56 |
| BCL2L12 | 1186334 | 2.85E−03 | 1.30 |
| MGC20781 | 731227 | 2.96E−03 | 1.49 |
| IMAGE:665649 | 665649 | 3.08E−03 | 1.66 |
| Transcribed | | | |
| locus | | | |
| IMAGE:1859532 | 1859532 | 3.11E−03 | 1.43 |
| DLX5 | 564878 | 0.00313 | 1.98 |
| LILRB5 | 71428 | 3.20E−03 | 1.73 |
| IMAGE:666279 | 666279 | 3.21E−03 | 2.30 |
| CDNA | | | |
| FLJ30779 fis, | | | |
| clone | | | |
| FEBRA2000815 | | | |
| PHC2 | 668146 | 3.23E−03 | 1.82 |
| MPST | 731241 | 3.28E−03 | 1.79 |
| IMAGE:2575 | 257555 | 3.32E−03 | 1.59 |
| KIAA1463 | 381118 | 3.39E−03 | 1.44 |
| ATP2B4 | 665814 | 3.43E−03 | 1.68 |
| MFAP3L | 726821 | 3.43E−03 | 1.35 |
| OSR1 | 364686 | 3.58E−03 | 1.84 |
| PRDX2 | 208439 | 3.74E−03 | 1.33 |
| HP | 82687 | 3.76E−03 | 1.46 |
| COX5A | 1085884 | 3.78E−03 | 1.67 |
| QIL1 | 727296 | 3.79E−03 | 1.32 |
| PCGF4 | 740457 | 3.90E−03 | 1.75 |
| IMAGE:667527 | 667527 | 3.94E−03 | 1.96 |
| LHB | 1671903 | 4.01E−03 | 1.60 |
| PLCG2 | 201467 | 4.13E−03 | 1.56 |
| TTC7B | 135379 | 4.23E−03 | 1.58 |
| FKBP6 | 795736 | 4.28E−03 | 1.55 |
| TFF1 | 1075949 | 4.28E−03 | 2.13 |
| H17 | 739432 | 4.31E−03 | 1.32 |
| IMAGE:363955 | 363955 | 4.31E−03 | 1.67 |
| Transcribed | | | |
| locus | | | |
| IMAGE:213529 | 213529 | 4.47E−03 | 1.62 |
| Transcribed | | | |
| locus | | | |
| SIGLEC7 | 743331 | 4.53E−03 | 1.71 |
| ANXA11 | 137238 | 4.53E−03 | 1.26 |
| IMAGE:743619 | 743619 | 4.59E−03 | 1.60 |
| ZNF258 | 1241974 | 4.73E−03 | 1.22 |
| COX6B1 | 632026 | 5.11E−03 | 1.39 |
| S100A16 | 730699 | 5.32E−03 | 1.34 |
| KIAA0759 | 743451 | 5.36E−03 | 2.52 |
| TRIP12 | 665820 | 5.38E−03 | 1.70 |
| MDH1 | 725188 | 5.49E−03 | 1.31 |
| MUTED | 731202 | 5.62E−03 | 1.98 |
| IMAGE:379937 | 379937 | 5.72E−03 | 1.80 |
| WIPI49 | 487148 | 5.74E−03 | 1.56 |
| BAK1 | 1288183 | 5.81E−03 | 2.02 |
| CREBBP | 172996 | 5.83E−03 | 1.75 |
| SIAT8C | 382069 | 5.88E−03 | 1.52 |
| HMG20A | 731277 | 5.90E−03 | 1.94 |
| VTN | 230126 | 5.96E−03 | 1.78 |
| NME7 | 743982 | 6.01E−03 | 2.37 |
| IMAGE:743579 | 743579 | 6.02E−03 | 2.02 |
| SDHA | 40304 | 6.05E−03 | 1.48 |
| SLC2A4RG | 376983 | 6.10E−03 | 1.42 |
| CTXN1 | 179266 | 6.29E−03 | 1.40 |
| LOC113655 | 40134 | 6.31E−03 | 1.36 |
| BAK1 | 235938 | 6.36E−03 | 1.77 |
| IMAGE:731689 | 731689 | 6.44E−03 | 1.30 |
| Transcribed | | | |
| locus | | | |
| SYCP2 | 136863 | 6.55E−03 | 2.05 |
| C20orf18 | 665433 | 6.64E−03 | 2.01 |
| IMAGE:727067 | 727067 | 6.69E−03 | 1.57 |
| Similar to | | | |
| hypothetical | | | |
| protein | | | |
| SB153 | | | |
| isoform 1 | | | |
| IMAGE:743078 | 743078 | 6.71E−03 | 1.39 |
| ETV4 | 809959 | 6.71E−03 | 1.50 |
| GALNT7 | 381854 | 6.94E−03 | 1.65 |
| TOSO | 813174 | 7.04E−03 | 1.58 |
| BMP5 | 1846326 | 0.00714 | 1.63 |
| MAK | 382002 | 7.18E−03 | 1.46 |
| JAK1 | 2030501 | 7.23E−03 | 1.71 |
| ATP11B | 666334 | 7.37E−03 | 2.27 |
| HRK | 767779 | 7.39E−03 | 1.99 |
| MCC | 731305 | 7.50E−03 | 2.48 |
| RABL5 | 471829 | 0.00755 | 1.27 |
| MAPRE2 | 383868 | 7.81E−03 | 2.35 |
| COX7B | 566862 | 8.32E−03 | 1.55 |
| IMAGE:731758 | 731758 | 8.39E−03 | 1.72 |
| Transcribed | | | |
| locus, weakly | | | |
| similar to | | | |
| NP_081070.1 | | | |
| kiaa-iso | | | |
| protein | | | |
| homolog; | | | |
| Band 47B | | | |
| [Mus | | | |
| musculus] | | | |
| MATN1 | 1624260 | 8.41E−03 | 1.44 |
| CD33 | 1917430 | 8.43E−03 | 1.72 |
| PPIL4 | 364777 | 8.46E−03 | 2.28 |
| DPF2 | 743519 | 0.00849 | 1.49 |
| FSHPRH1 | 667355 | 8.90E−03 | 1.40 |
| ZNF319 | 364729 | 8.93E−03 | 2.52 |
| RHOD | 591907 | 9.04E−03 | 1.29 |
| MESP1 | 25865 | 0.00912 | 1.20 |
| CASP10 | 241481 | 9.15E−03 | 1.98 |
| ALDH3A1 | 525221 | 9.76E−03 | 1.81 |
| KIAA0427 | 127507 | 9.77E−03 | 1.35 |
| GCAT | 307094 | 9.92E−03 | 1.26 |
| LOC339903 | 135352 | 9.92E−03 | 1.52 |
| C22orf16 | 236119 | 9.93E−03 | 1.46 |
| NDUFS3 | 743811 | 9.99E−03 | 1.24 |
| GNAS | 382791 | 1.00E−02 | 1.90 |
| IMAGE:742061 | 742061 | 1.00E−02 | 1.60 |
| MAPK3 | 323438 | 1.00E−02 | 1.62 |
| FLJ21159 | 251147 | 1.02E−02 | 1.27 |
| APIN | 364706 | 1.02E−02 | 1.83 |
| LRRIQ2 | 487152 | 1.07E−02 | 1.54 |
| MRPL45 | 364717 | 1.11E−02 | 1.66 |
| IMAGE:136686 | 136686 | 1.14E−02 | 1.36 |
| LOC284669 | 364885 | 1.18E−02 | 1.56 |
| ATF4 | 178348 | 1.22E−02 | 1.34 |
| GRIK2 | 731363 | 1.24E−02 | 1.32 |
| CLIC4 | 666085 | 1.25E−02 | 1.41 |
| PLCG1 | 1174287 | 1.28E−02 | 1.79 |
| KIF27 | 667657 | 1.29E−02 | 1.35 |
| CXCL13 | 347362 | 1.30E−02 | 1.37 |
| DDX54 | 743268 | 1.34E−02 | 1.35 |
| GJA5 | 196338 | 1.36E−02 | 1.68 |
| C6orf176 | 173200 | 1.37E−02 | 1.39 |
| ALDH1A3 | 486189 | 1.37E−02 | 1.99 |
| IMAGE:665403 | 665403 | 1.37E−02 | 1.52 |
| Transcribed | | | |
| locus | | | |
| CCDC6 | 487848 | 1.38E−02 | 1.40 |
| IMAGE:745512 | 745512 | 1.43E−02 | 1.38 |
| GLRX2 | 731044 | 1.45E−02 | 1.39 |
| NIFIE14 | 472160 | 1.51E−02 | 1.26 |
| C10orf99 | 713624 | 1.53E−02 | 1.22 |
| C15orf29 | 177775 | 1.53E−02 | 1.46 |
| SEPW1 | 173628 | 1.60E−02 | 2.05 |
| UBE2D2 | 594655 | 1.63E−02 | 1.27 |
| IMAGE:744616 | 744616 | 1.64E−02 | 1.40 |
| DNA | | | |
| damage | | | |
| repair and | | | |
| recombination | | | |
| protein | | | |
| RAD52 | | | |
| pseudogene | | | |
| IMAGE:364932 | 364932 | 1.65E−02 | 1.44 |
| CDNA | | | |
| FLJ35491 fis, | | | |
| clone | | | |
| SMINT2008625, | | | |
| moderately | | | |
| similar to | | | |
| GLYCINE | | | |
| CLEAVAGE | | | |
| SYSTEM H | | | |
| PROTEIN | | | |
| PRECURSOR | | | |
| PRKCDBP | 157847 | 1.67E−02 | 1.44 |
| IMAGE:744899 | 744899 | 1.67E−02 | 1.56 |
| Homo | | | |
| sapiens, | | | |
| clone | | | |
| IMAGE:4837072, | | | |
| mRNA | | | |
| C4orf16 | 681890 | 1.67E−02 | 1.32 |
| IMAGE:745133 | 745133 | 1.70E−02 | 1.21 |
| Transcribed | | | |
| locus | | | |
| EIF4EBP1 | 713608 | 1.71E−02 | 1.38 |
| ATP5G3 | 740090 | 1.74E−02 | 1.37 |
| MAD2L2 | 727178 | 1.74E−02 | 1.35 |
| DMD | 743394 | 1.75E−02 | 1.74 |
| MRPS11 | 471574 | 1.82E−02 | 1.80 |
| FBXO46 | 471664 | 1.82E−02 | 1.61 |
| RASAL2 | 486304 | 1.86E−02 | 1.95 |
| C6orf118 | 731745 | 1.88E−02 | 1.34 |
| SNX8 | 366585 | 1.96E−02 | 1.29 |
| ABR | 1012903 | 1.97E−02 | 1.35 |
| IMAGE:24587 | 24587 | 2.03E−02 | 1.31 |
| IMAGE:742837 | 742837 | 2.03E−02 | 1.58 |
| Transcribed | | | |
| locus | | | |
| ELK4 | 236155 | 2.07E−02 | 1.27 |
| NME1 | 726600 | 2.13E−02 | 1.48 |
| GAPD | 152847 | 2.13E−02 | 1.54 |
| OR7E38P | 486540 | 2.17E−02 | 1.22 |
| MGC29784 | 731019 | 2.22E−02 | 1.19 |
| PIP | 985457 | 2.28E−02 | 1.26 |
| IMAGE:383718 | 383718 | 2.36E−02 | 1.51 |
| AD023 | 731645 | 2.37E−02 | 1.29 |
| PML | 724554 | 2.45E−02 | 1.17 |
| UQCRC1 | 714414 | 2.46E−02 | 1.42 |
| CBL | 1578721 | 2.49E−02 | 1.30 |
| CGI-128 | 786662 | 2.51E−02 | 1.24 |
| IMAGE:383966 | 383966 | 0.02518 | 1.31 |
| CIRH1A | 261500 | 2.55E−02 | 1.24 |
| DUSP3 | 119772 | 2.65E−02 | 1.28 |
| PDXP | 743182 | 2.81E−02 | 1.28 |
| DGKQ | 366233 | 2.90E−02 | 1.19 |
| KIAA1277 | 364822 | 2.94E−02 | 1.50 |
| AMD1 | 132752 | 2.95E−02 | 1.17 |
| LHFPL2 | 364356 | 2.98E−02 | 1.46 |
| PCSK6 | 31924 | 2.99E−02 | 2.16 |
| KIAA1280 | 366085 | 2.99E−02 | 1.23 |
| SLIT1 | 38403 | 3.10E−02 | 1.26 |
| FLJ22471 | 668510 | 3.10E−02 | 2.44 |
| CD8B1 | 1743279 | 3.27E−02 | 1.19 |
| IMAGE:743290 | 743290 | 3.35E−02 | 2.01 |
| Transcribed | | | |
| locus | | | |
| RUNX1 | 263251 | 3.39E−02 | 1.42 |
| SYP | 30471 | 3.40E−02 | 2.11 |
| PPAP2C | 486447 | 3.41E−02 | 1.33 |
| PINK1 | 729929 | 3.44E−02 | 1.30 |
| TYMS | 2242054 | 3.49E−02 | 1.22 |
| IMAGE:382521 | 382521 | 3.52E−02 | 1.54 |
| DIO1 | 296702 | 3.53E−02 | 1.45 |
| C1QBP | 173371 | 3.57E−02 | 1.52 |
| PSMD11 | 383945 | 3.57E−02 | 1.54 |
| LAD1 | 121551 | 3.59E−02 | 1.17 |
| MIF | 179133 | 3.59E−02 | 1.26 |
| CCL28 | 136919 | 3.61E−02 | 1.21 |
| PTPN4 | 666367 | 3.61E−02 | 1.40 |
| DUSP24 | 740158 | 3.70E−02 | 1.57 |
| IMAGE:682585 | 682585 | 3.86E−02 | 1.27 |
| SPI1 | 1285305 | 3.92E−02 | 1.34 |
| DECR2 | 730942 | 3.93E−02 | 1.39 |
| ABCA8 | 284828 | 3.95E−02 | 1.45 |
| HSPC072 | 668329 | 0.04017 | 1.73 |
| SAFB | 42280 | 4.07E−02 | 2.48 |
| IMAGE:136121 | 136121 | 4.07E−02 | 1.55 |
| CDNA | | | |
| FLJ10247 fis, | | | |
| clone | | | |
| HEMBB1000705 | | | |
| DOK5 | 25664 | 4.11E−02 | 1.56 |
| TGFB3 | 1561035 | 4.20E−02 | 1.32 |
| IRAK1 | 379200 | 4.23E−02 | 1.16 |
| EPRS | 669549 | 0.04334 | 1.16 |
| FLJ23577 | 668452 | 4.35E−02 | 2.08 |
| HTATIP2 | 726618 | 4.36E−02 | 1.18 |
| PANK1 | 256177 | 4.38E−02 | 1.20 |
| DCXR | 724596 | 4.46E−02 | 1.22 |
| KCNK4 | 743016 | 4.52E−02 | 1.36 |
| ITGA5 | 135671 | 4.55E−02 | 1.28 |
| SYT7 | 177827 | 0.04549 | 1.53 |
| NDUFA7 | 364469 | 4.59E−02 | 1.21 |
| VIL2 | 124701 | 4.66E−02 | 1.26 |
| FLJ35630 | 485652 | 4.66E−02 | 1.21 |
| MLPH | 667259 | 4.73E−02 | 1.76 |
| RAB8A | 712118 | 4.81E−02 | 1.31 |
| SFN | 346610 | 4.83E−02 | 1.16 |
| FLJ12517 | 743220 | 4.86E−02 | 1.14 |
| CLGN | 1049033 | 0.04934 | 1.66 |

TABLE 19

| | | OTA- | |
|---|---|---|---|
| | | atypical | Under |
| name | cloid | p-value | fold |
| ZNF76 | 745003 | 7.8753E−23 | 0.60 |
| POLR2A | 740130 | 3.2874E−20 | 0.58 |
| RODH | 471641 | 9.42E−18 | 0.13 |
| LOC126669 | 682088 | 1.5036E−17 | 0.59 |
| LOC340073 | 731404 | 5.6235E−17 | 0.47 |
| IMAGE:2254555 | 2254555 | 5.0996E−16 | 0.58 |
| CDKN1C | 2381770 | 1.0691E−13 | 0.57 |
| CDH1 | 214008 | 2.0729E−13 | 0.38 |
| CLDN7 | 300268 | 3.277E−13 | 0.64 |
| ZBTB16 | 2467442 | 1.2466E−10 | 0.40 |
| CD63 | 125552 | 3.0515E−09 | 0.72 |
| C21orf55 | 666517 | 8.6603E−09 | 0.65 |
| RNF123 | 366159 | 1.3739E−08 | 0.76 |
| IMAGE:740927 | 740927 | 2.5316E−08 | 0.57 |
| CFH | 665784 | 4.6026E−08 | 0.37 |
| BAT3 | 24392 | 3.2661E−07 | 0.73 |
| RBBP7 | 727457 | 5.0481E−07 | 0.87 |
| GATA3 | 148796 | 5.6774E−07 | 0.55 |
| MGP | 590264 | 5.8207E−07 | 0.33 |
| APOL1 | 665632 | 8.5144E−07 | 0.40 |
| CXCL14 | 345034 | 1.0689E−06 | 0.15 |
| CCL5 | 840753 | 3.0169E−06 | 0.44 |
| CAV1 | 309645 | 3.7112E−06 | 0.25 |
| TIE1 | 743043 | 4.493E−06 | 0.71 |
| LOC286076 | 731275 | 5.0989E−06 | 0.67 |
| ESRRBL1 | 51232 | 9.4953E−06 | 0.69 |
| SOSTDC1 | 667048 | 1.0076E−05 | 0.81 |
| APOD | 838611 | 1.4867E−05 | 0.25 |
| GPNMB | 773330 | 1.5722E−05 | 0.26 |
| HMGB2 | 884365 | 2.4273E−05 | 0.50 |
| TP53I11 | 667514 | 3.3387E−05 | 0.48 |
| DHRS6 | 364412 | 7.2289E−05 | 0.71 |
| PLAC9 | 366115 | 7.6205E−05 | 0.46 |
| CAPN3 | 757248 | 0.00010028 | 0.61 |
| FRZB | 146049 | 0.00010878 | 0.56 |
| COL1A1 | 140347 | 0.00011911 | 0.26 |
| BCL2 | 342181 | 0.00013964 | 0.65 |
| IMAGE:727123 | 727123 | 0.00016327 | 0.75 |
| Transcribed | | | |
| locus, | | | |
| strongly | | | |
| similar to | | | |
| NP_004491.1 | | | |
| heterogeneous | | | |
| nuclear | | | |
| ribonucleoprotein | | | |
| C isoform | | | |
| b; | | | |
| nuclear | | | |
| ribonucleoprotein | | | |
| particle | | | |
| C2 | | | |
| protein; | | | |
| nuclear | | | |
| ribonucleoprotein | | | |
| particle | | | |
| C1 | | | |
| protein | | | |
| [Homo | | | |
| sapiens] | | | |
| LRP1 | 30219 | 0.00018822 | 0.52 |
| MMP2 | 155839 | 0.00020213 | 0.42 |
| NUP214 | 382612 | 0.0002167 | 0.53 |
| SPON2 | 723923 | 0.00025027 | 0.73 |
| IGSF10 | 682276 | 0.00026272 | 0.65 |
| TBC1D22A | 682479 | 0.00027374 | 0.73 |
| DKFZp434M202 | 743118 | 0.00029492 | 0.69 |
| C10orf116 | 740941 | 0.00029723 | 0.40 |
| CD5 | 356841 | 0.00035219 | 0.75 |
| IMAGE:53081 | 53081 | 0.00035776 | 0.78 |
| TSC | 745490 | 0.0003911 | 0.42 |
| ANKRD28 | 687381 | 0.00040418 | 0.55 |
| CCND1 | 324079 | 0.00045244 | 0.69 |
| IMAGE:173818 | 173818 | 0.00050225 | 0.81 |
| Transcribed | | | |
| locus | | | |
| MGAT3 | 731060 | 0.00051388 | 0.57 |
| NCB5OR | 743367 | 0.00053407 | 0.55 |
| SPTLC2 | 365000 | 0.00057248 | 0.79 |
| PRKCQ | 669149 | 0.00057256 | 0.53 |
| SFRP4 | 285693 | 0.00057595 | 0.48 |
| IMAGE:731398 | 731398 | 0.00067333 | 0.67 |
| PPP3CC | 110481 | 0.00069215 | 0.74 |
| IQCF3 | 726578 | 0.00080815 | 0.68 |
| ARHGEF6 | 687990 | 0.00086388 | 0.66 |
| TIAM1 | 23612 | 0.00089109 | 0.31 |
| IMAGE:723752 | 723752 | 0.00092229 | 0.68 |
| AQP11 | 713831 | 0.00092762 | 0.47 |
| CD3E | 1536968 | 0.00093072 | 0.45 |
| NT5C2 | 725076 | 0.00099636 | 0.57 |
| LYPLA3 | 471702 | 0.00102036 | 0.58 |
| S100A10 | 119939 | 0.00120361 | 0.56 |
| SEPT-05 | 448163 | 0.0012145 | 0.26 |
| GLT8D2 | 365271 | 0.00131886 | 0.39 |
| HDAC9 | 738506 | 0.00143075 | 0.72 |
| BDKRB2 | 665674 | 0.00144781 | 0.51 |
| DF | 666128 | 0.00145035 | 0.26 |
| NFE2L2 | 29541 | 0.00153006 | 0.77 |
| FXYD1 | 204686 | 0.00159585 | 0.67 |
| IMAGE:731665 | 731665 | 0.00167237 | 0.54 |
| RANBP10 | 682251 | 0.00172358 | 0.72 |
| LOC284371 | 724126 | 0.00245517 | 0.71 |
| IMAGE:22908 | 22908 | 0.00250266 | 0.76 |
| Transcribed | | | |
| locus, | | | |
| weakly | | | |
| similar to | | | |
| NP_775735.1 | | | |
| I(3)mbt- | | | |
| like 4 | | | |
| [Homo | | | |
| sapiens] | | | |
| PLA2G2A | 152802 | 0.00253136 | 0.70 |
| CASP4 | 356960 | 0.00285061 | 0.83 |
| EVI5L | 173119 | 0.00338218 | 0.63 |
| APOL3 | 366289 | 0.00370487 | 0.58 |
| AMPD2 | 669141 | 0.00384401 | 0.82 |
| PLXNB2 | 1420676 | 0.00409831 | 0.58 |
| RDH5 | 682109 | 0.00428632 | 0.62 |
| ECM1 | 301122 | 0.0044458 | 0.27 |
| CD47 | 357442 | 0.00479452 | 0.62 |
| STYX | 174879 | 0.00490228 | 0.58 |
| S100A4 | 868577 | 0.00504892 | 0.35 |
| ASGR1 | 25883 | 0.00527851 | 0.44 |
| SLAMF8 | 288807 | 0.00568981 | 0.70 |
| PP784 | 366089 | 0.00582582 | 0.66 |
| IMAGE:471852 | 471852 | 0.005834 | 0.73 |
| CDNA | | | |
| FLJ4006 | | | |
| TIMP1 | 162246 | 0.00610248 | 0.40 |
| LGALS3BP | 742100 | 0.0066389 | 0.36 |
| IMAGE:743722 | 743722 | 0.00674466 | 0.66 |
| MGC40214 | 666455 | 0.00721803 | 0.78 |
| CITED1 | 265558 | 0.00748256 | 0.43 |
| FLJ14351 | 744943 | 0.00769555 | 0.30 |
| IMAGE:379279 | 379279 | 0.00786189 | 0.64 |
| IMAGE:668924 | 668924 | 0.00797669 | 0.71 |
| ACTB | 203166 | 0.00896929 | 0.86 |
| FLJ21918 | 194302 | 0.00901426 | 0.67 |
| SNRP70 | 729971 | 0.00908157 | 0.56 |
| C4A | 724366 | 0.01003098 | 0.46 |
| IMAGE:682311 | 682311 | 0.01031247 | 0.74 |
| LOC440722 | | | |
| ITPR1 | 667348 | 0.0106412 | 0.59 |
| SIAT4B | 365877 | 0.01066903 | 0.62 |
| C14orf140 | 731423 | 0.01082785 | 0.77 |
| C7orf27 | 713859 | 0.0108452 | 0.76 |
| SMARCA5 | 730037 | 0.01143683 | 0.65 |
| FOXM1 | 23641 | 0.01240416 | 0.57 |
| FLJ20674 | 713837 | 0.01347485 | 0.86 |
| MTUS1 | 376597 | 0.01348587 | 0.64 |
| IMAGE:135125 | 135125 | 0.01360246 | 0.58 |
| SGSH | 669263 | 0.01424674 | 0.53 |
| IFNAR2 | 123950 | 0.01445371 | 0.83 |
| EPHB6 | 172982 | 0.0150956 | 0.32 |
| CAV1 | 1911930 | 0.0153774 | 0.38 |
| CAPNS1 | 152655 | 0.01547068 | 0.73 |
| RUNX3 | 122874 | 0.0160785 | 0.64 |
| GADD45GIP1 | 1084386 | 0.0161436 | 0.73 |
| GJA1 | 839101 | 0.01616925 | 0.53 |
| IMAGE:251427 | 251427 | 0.01673194 | 0.77 |
| FBXO21 | 666168 | 0.01684513 | 0.65 |
| SLC9A5 | 112819 | 0.01735236 | 0.69 |
| TSHR | 565317 | 0.01748004 | 0.48 |
| SPDEF | 1188588 | 0.01788258 | 0.85 |
| KRT17 | 366889 | 0.01859126 | 0.56 |
| IMAGE:743485 | 743485 | 0.0187952 | 0.69 |
| Similar to | | | |
| RIKEN | | | |
| cDNA | | | |
| E130306M17 | | | |
| gene | | | |
| HN1 | 471568 | 0.01882732 | 0.70 |
| IMAGE:744505 | 744505 | 0.02002197 | 0.73 |
| IQCA | 669510 | 0.02005822 | 0.58 |
| LOC286272 | 667361 | 0.02077784 | 0.71 |
| PDLIM1 | 135689 | 0.02118121 | 0.45 |
| LOC91526 | 666564 | 0.0217601 | 0.76 |
| IMAGE:738332 | 738332 | 0.02220518 | 0.29 |
| Transcribed | | | |
| locus | | | |
| IMAGE:23765 | 23765 | 0.02343381 | 0.68 |
| NYREN18 | 668584 | 0.02345468 | 0.80 |
| PIGV | 669319 | 0.02466844 | 0.79 |
| PLA2G6 | 744087 | 0.02496728 | 0.48 |
| POU2F2 | 188393 | 0.02538555 | 0.68 |
| PRKG1 | 667587 | 0.02549526 | 0.75 |
| SOD3 | 795309 | 0.02637889 | 0.38 |
| DAXX | 292042 | 0.02655992 | 0.75 |
| TM4SF9 | 812967 | 0.02671422 | 0.50 |
| YTHDF3 | 135450 | 0.02725943 | 0.66 |
| CCND3 | 327182 | 0.02743722 | 0.87 |
| IMAGE:666946 | 666946 | 0.02807361 | 0.74 |
| C20orf19 | 366032 | 0.02845889 | 0.64 |
| CCL19 | 1707527 | 0.02868686 | 0.38 |
| SNTB2 | 667310 | 0.02953683 | 0.87 |
| TOE1 | 52897 | 0.02974582 | 0.65 |
| GPC3 | 137131 | 0.029841 | 0.65 |
| PBXIP1 | 366042 | 0.0300236 | 0.49 |
| KIAA1196 | 738938 | 0.03031142 | 0.81 |
| IMAGE:725726 | 725726 | 0.03126401 | 0.58 |
| Hypothetical | | | |
| gene | | | |
| supported | | | |
| by | | | |
| AK093801 | | | |
| RIPK3 | 667313 | 0.03128138 | 0.63 |
| IMAGE:665392 | 665392 | 0.03169622 | 0.61 |
| Hypothetical | | | |
| LOC388790 | | | |
| BMP5 | 1846326 | 0.03227381 | 0.72 |
| TCEA2 | 730149 | 0.03292087 | 0.76 |
| ZFOC1 | 744606 | 0.0347902 | 0.67 |
| CKLFSF3 | 489249 | 0.03501322 | 0.71 |
| SENP6 | 739237 | 0.0350721 | 0.62 |
| LEMD1 | 731047 | 0.03610772 | 0.58 |
| PSPC1 | 687541 | 0.03646334 | 0.90 |
| NPFF | 365161 | 0.03913586 | 0.58 |
| THAP3 | 687800 | 0.0399181 | 0.58 |
| CCNE1 | 68950 | 0.04140347 | 0.50 |
| IMMP2L | 136260 | 0.04147879 | 0.86 |
| FOXO3A | 109142 | 0.04212529 | 0.61 |
| BMP4 | 69166 | 0.04398031 | 0.63 |
| CCNE1 | 357807 | 0.04398241 | 0.64 |
| SULT1E1 | 207926 | 0.04421397 | 0.74 |
| KLRB1 | 2091591 | 0.04434553 | 0.72 |
| FGF4 | 1550616 | 0.04512349 | 0.59 |
| TUSC1 | 740381 | 0.04739201 | 0.67 |
| IMAGE:731714 | 731714 | 0.04824836 | 0.59 |
| CDNA | | | |
| FLJ26188 | | | |
| fis, | | | |
| clone | | | |
| ADG04821 | | | |
| FLJ10707 | 364575 | 0.04833655 | 0.83 |
| TRAF5 | 1286238 | 0.04846001 | 0.70 |
| FMO5 | 364526 | 0.04864895 | 0.72 |
| FOXO1A | 151247 | 0.0489662 | 0.57 |

TABLE 20

| | OTA- | | |
|---|---|---|---|
| | atypical | Over | |
| name | cloid | p-value | fold |
| IMAGE:724556 | 724556 | 5.95E−07 | 1.25 |
| Transcribed | | | |
| locus | | | |
| HSPC072 | 668329 | 2.07E−04 | 1.40 |
| C6orf176 | 173200 | 1.24E−03 | 1.75 |
| KNS2 | 174654 | 3.56E−03 | 1.13 |
| MGC20781 | 731227 | 4.21E−03 | 1.22 |
| MATN1 | 1624260 | 1.44E−02 | 1.52 |
| IMAGE:743078 | 743078 | 1.47E−02 | 1.17 |
| FBXW5 | 739576 | 2.20E−02 | 1.28 |
| IL2RA | 3054031 | 3.15E−02 | 1.32 |
| DUSP3 | 119772 | 4.06E−02 | 1.66 |

TABLE 21

| | OTC | Under | |
|---|---|---|---|
| name | cloid | p-value | fold |
| IMAGE:727123 | 727123 | 2.7617E−23 | 0.68 |
| Transcribed | | | |
| locus, | | | |
| strongly | | | |
| similar to | | | |
| NP_004491.1 | | | |
| heterogeneous | | | |
| nuclear | | | |
| ribonucleoprotein C | | | |
| isoform b; | | | |
| nuclear | | | |
| ribonucleoprotein | | | |
| particle | | | |
| C2 protein; | | | |
| nuclear | | | |
| ribonucleoprotein | | | |
| particle | | | |
| C1 protein | | | |
| [Homo | | | |
| sapiens] | | | |
| C4A | 724366 | 6.3678E−20 | 0.11 |
| APOL3 | 366289 | 4.4335E−16 | 0.39 |
| RODH | 471641 | 3.0605E−14 | 0.24 |
| TIMP3 | 501476 | 3.8968E−14 | 0.35 |
| FLJ14351 | 744943 | 1.8705E−13 | 0.12 |
| EPHA4 | 2237263 | 3.9675E−13 | 0.44 |
| MMP2 | 155839 | 5.2372E−13 | 0.32 |
| COL1A1 | 140347 | 1.232E−12 | 0.22 |
| IMP-2 | 743774 | 1.8908E−12 | 0.27 |
| IMAGE:1716286 | 1716286 | 6.4804E−12 | 0.26 |
| GLT8D2 | 365271 | 2.3704E−11 | 0.28 |
| SPINL | 236399 | 3.4967E−11 | 0.33 |
| C14orf140 | 731423 | 4.4437E−11 | 0.78 |
| LOC120224 | 252291 | 4.4649E−11 | 0.60 |
| TM4SF9 | 812967 | 5.8913E−11 | 0.51 |
| MGC99813 | 739097 | 1.1187E−10 | 0.20 |
| LYPLA3 | 471702 | 1.5636E−10 | 0.70 |
| PLXNB2 | 1420676 | 1.9854E−10 | 0.34 |
| CRABP1 | 739193 | 2.1378E−10 | 0.14 |
| PREB | 740347 | 2.692E−10 | 0.23 |
| FRMD4B | 669564 | 8.1194E−10 | 0.51 |
| BAZ2B | 609631 | 8.1218E−10 | 0.63 |
| IMAGE:731751 | 731751 | 1.6927E−09 | 0.48 |
| Transcribed | | | |
| locus | | | |
| IMAGE:666671 | 666671 | 1.1003E−08 | 0.58 |
| Transcribed | | | |
| locus | | | |
| TSC | 745490 | 1.8198E−08 | 0.27 |
| SFRP4 | 285693 | 2.4749E−08 | 0.35 |
| MRC2 | 235882 | 1.2867E−07 | 0.39 |
| CICE | 725791 | 1.6529E−07 | 0.49 |
| TIAM1 | 23612 | 1.8313E−07 | 0.35 |
| CXCL14 | 345034 | 1.8994E−07 | 0.08 |
| NBL1 | 503874 | 1.9736E−07 | 0.24 |
| APOD | 838611 | 3.0261E−07 | 0.18 |
| MT1F | 81911 | 3.9725E−07 | 0.18 |
| HBA2 | 469647 | 4.0621E−07 | 0.22 |
| TP53I11 | 667514 | 4.8536E−07 | 0.46 |
| INHBB | 730012 | 6.3101E−07 | 0.35 |
| SPATA12 | 730300 | 6.4449E−07 | 0.60 |
| C10orf116 | 740941 | 1.0112E−06 | 0.25 |
| HOXB6 | 738358 | 1.8287E−06 | 0.76 |
| GPR124 | 486493 | 2.4789E−06 | 0.50 |
| URB | 809719 | 2.7159E−06 | 0.54 |
| LAPTM4A | 726684 | 3.1145E−06 | 0.47 |
| IMAGE:687667 | 687667 | 3.4863E−06 | 0.30 |
| CDNA | | | |
| clone | | | |
| IMAGE:4811759, | | | |
| partial | | | |
| cds | | | |
| TIMP1 | 162246 | 3.5649E−06 | 0.26 |
| TPM2 | 740620 | 3.6412E−06 | 0.27 |
| SENP6 | 366436 | 5.3738E−06 | 0.55 |
| SALL4 | 726454 | 5.8869E−06 | 0.35 |
| HBB | 469549 | 8.147E−06 | 0.25 |
| ETF1 | 146976 | 9.4951E−06 | 0.44 |
| CFH | 665784 | 1.0597E−05 | 0.40 |
| RFX2 | 731738 | 1.0616E−05 | 0.48 |
| HNRPA3 | 365349 | 1.0734E−05 | 0.65 |
| MGC5395 | 238840 | 1.0969E−05 | 0.40 |
| C4A | 491004 | 1.1538E−05 | 0.33 |
| RUNX3 | 122874 | 1.5173E−05 | 0.60 |
| IMAGE:46991 | 46991 | 1.5437E−05 | 0.44 |
| Similar to | | | |
| breakpoint | | | |
| cluster region | | | |
| isoform 1 | | | |
| CDH16 | 726763 | 1.6824E−05 | 0.33 |
| MT1F | 78353 | 1.9038E−05 | 0.15 |
| IMAGE:738896 | 738896 | 1.9575E−05 | 0.15 |
| FBXL16 | 178908 | 2.0945E−05 | 0.80 |
| NFIB | 416959 | 2.1672E−05 | 0.69 |
| ZNF251 | 486401 | 2.9225E−05 | 0.25 |
| APOL1 | 665632 | 2.923E−05 | 0.45 |
| RP4-622L5 | 742590 | 2.9406E−05 | 0.57 |
| GIMAP5 | 180259 | 3.1108E−05 | 0.45 |
| MGC40214 | 666455 | 3.4186E−05 | 0.61 |
| RPS6KA2 | 22711 | 3.7532E−05 | 0.50 |
| BDKRB2 | 665674 | 3.7744E−05 | 0.36 |
| GATA3 | 148796 | 3.7841E−05 | 0.37 |
| CERKL | 489326 | 3.7966E−05 | 0.55 |
| ASGR1 | 25883 | 3.8987E−05 | 0.45 |
| GNAI2 | 724306 | 4.1649E−05 | 0.44 |
| ZNF76 | 745003 | 4.1776E−05 | 0.43 |
| CLDN1 | 594279 | 4.3619E−05 | 0.25 |
| IMAGE:342256 | 342256 | 5.2243E−05 | 0.15 |
| SIN3A | 26455 | 5.6921E−05 | 0.61 |
| CKLFSF4 | 143759 | 5.7456E−05 | 0.79 |
| KIAA0515 | 687852 | 7.835E−05 | 0.52 |
| LASP1 | 592598 | 8.0253E−05 | 0.35 |
| MGC17299 | 713422 | 8.7659E−05 | 0.55 |
| CaMKIINalpha | 173820 | 9.6232E−05 | 0.49 |
| VPS13D | 136560 | 0.00011233 | 0.58 |
| GABRE | 209137 | 0.00011566 | 0.56 |
| TNFSF12 | 136361 | 0.00011734 | 0.64 |
| IMAGE:743205 | 743205 | 0.00011953 | 0.45 |
| PBXIP1 | 366042 | 0.00012575 | 0.41 |
| ARID1B | 665538 | 0.00013937 | 0.88 |
| SYN3 | 727056 | 0.00015868 | 0.41 |
| ABCB1 | 1837488 | 0.00015965 | 0.54 |
| DNALI1 | 782688 | 0.00017057 | 0.23 |
| PLA2G6 | 744087 | 0.00017386 | 0.35 |
| SLC6A8 | 725877 | 0.00018797 | 0.26 |
| PIK3CA | 2238108 | 0.0001957 | 0.37 |
| PPP1CA | 257259 | 0.00019642 | 0.71 |
| LRP5 | 725836 | 0.00020223 | 0.55 |
| PDE8A | 666154 | 0.00021562 | 0.62 |
| FLJ27523 | 743246 | 0.00022705 | 0.42 |
| RAP1GA1 | 971276 | 0.00023382 | 0.61 |
| SEPT-05 | 448163 | 0.000238 | 0.20 |
| DCN | 666410 | 0.00024122 | 0.29 |
| POLI | 137720 | 0.00024263 | 0.46 |
| CABIN1 | 1844968 | 0.0002434 | 0.40 |
| TNFRSF1B | 73703 | 0.00024434 | 0.58 |
| CD34 | 770858 | 0.00024685 | 0.51 |
| LRP2 | 2055272 | 0.00025885 | 0.40 |
| NOG | 487474 | 0.00027732 | 0.72 |
| WDR5 | 731023 | 0.00030289 | 0.75 |
| ITPR1 | 667348 | 0.00033483 | 0.53 |
| BAI3 | 50491 | 0.00034402 | 0.59 |
| POLR2A | 740130 | 0.00034704 | 0.41 |
| HIS1 | 342551 | 0.0003806 | 0.65 |
| IFITM3 | 713623 | 0.00038781 | 0.42 |
| LILRA1 | 2032639 | 0.0004104 | 0.50 |
| SPON2 | 723923 | 0.00044302 | 0.72 |
| KLRD1 | 2728204 | 0.00045592 | 0.57 |
| IMAGE:176543 | 176543 | 0.00046548 | 0.56 |
| GATA4 | 781738 | 0.00048721 | 0.63 |
| GJA1 | 839101 | 0.00054374 | 0.55 |
| SLA | 32339 | 0.00055491 | 0.48 |
| IMAGE:731357 | 731357 | 0.00055529 | 0.78 |
| Transcribed | | | |
| locus | | | |
| DJ462O23.2 | 738970 | 0.00060349 | 0.52 |
| FLJ35794 | 364204 | 0.00060589 | 0.56 |
| IMAGE:1089025 | 1089025 | 0.0007481 | 0.28 |
| FLJ10707 | 364575 | 0.00076696 | 0.45 |
| LOC388886 | 34007 | 0.00076698 | 0.52 |
| FLJ20254 | 135096 | 0.00078261 | 0.56 |
| DHRS6 | 364412 | 0.00080511 | 0.46 |
| TPD52 | 743259 | 0.00081655 | 0.21 |
| APOE | 1870594 | 0.0008476 | 0.47 |
| IL11RA | 1101773 | 0.00099007 | 0.45 |
| DGKQ | 366233 | 0.00099155 | 0.66 |
| CCL15 | 342290 | 0.00110731 | 0.39 |
| CDR2 | 366067 | 0.00112025 | 0.86 |
| TSHR | 565317 | 0.00115444 | 0.46 |
| IMAGE:742853 | 742853 | 0.00116933 | 0.59 |
| NFE2L2 | 29541 | 0.00119931 | 0.55 |
| ATXN1 | 682528 | 0.00121938 | 0.47 |
| MLL | 80688 | 0.00127262 | 0.45 |
| IMAGE:731398 | 731398 | 0.00132903 | 0.54 |
| AQP11 | 713831 | 0.00135026 | 0.55 |
| ALDH1A1 | 309697 | 0.00135541 | 0.46 |
| ZNF395 | 744983 | 0.00139312 | 0.56 |
| TLN1 | 187482 | 0.00151133 | 0.41 |
| HCLS1 | 665452 | 0.00155934 | 0.62 |
| PDCD4 | 665376 | 0.00163269 | 0.51 |
| CDH3 | 359051 | 0.00167179 | 0.53 |
| PDGFRL | 139242 | 0.00172177 | 0.49 |
| CCL19 | 1707527 | 0.00177893 | 0.16 |
| IMAGE:731726 | 731726 | 0.00179977 | 0.32 |
| Transcribed | | | |
| locus | | | |
| IMAGE:145112 | 145112 | 0.0018241 | 0.60 |
| IMAGE:744055 | 744055 | 0.00184821 | 0.53 |
| Transcribed | | | |
| locus | | | |
| CA11 | 282587 | 0.00186777 | 0.56 |
| MBNL1 | 136114 | 0.00190617 | 0.49 |
| GCLC | 1526058 | 0.00195642 | 0.55 |
| PLAC9 | 366115 | 0.00200838 | 0.41 |
| IFNAR2 | 123950 | 0.0023353 | 0.66 |
| TIE1 | 743043 | 0.00253709 | 0.65 |
| CTBP2 | 416744 | 0.00258981 | 0.73 |
| CD3E | 1536968 | 0.0026095 | 0.40 |
| CAV1 | 309645 | 0.00270324 | 0.31 |
| IMAGE:738332 | 738332 | 0.00290812 | 0.48 |
| Transcribed | | | |
| locus | | | |
| KLHL9 | 471696 | 0.00300852 | 0.53 |
| CITED2 | 365090 | 0.00301673 | 0.53 |
| SNTB2 | 667310 | 0.00308273 | 0.61 |
| FABP5 | 1088781 | 0.00315076 | 0.38 |
| GATA2 | 149809 | 0.00322922 | 0.74 |
| EVI5L | 173119 | 0.00323442 | 0.59 |
| IMAGE:724416 | 724416 | 0.00338129 | 0.41 |
| Transcribed | | | |
| locus | | | |
| FAM38B | 743146 | 0.00379144 | 0.69 |
| FOXO3A | 109142 | 0.00389287 | 0.46 |
| SELE | 1239535 | 0.00404397 | 0.57 |
| FLT4 | 668815 | 0.00404811 | 0.52 |
| SATB1 | 364510 | 0.00409049 | 0.50 |
| KIAA1196 | 738938 | 0.00430547 | 0.64 |
| SLC25A23 | 741954 | 0.00436505 | 0.53 |
| ZNF580 | 668089 | 0.00454562 | 0.52 |
| FOXO1A | 151247 | 0.00472685 | 0.58 |
| SEPT-02 | 729542 | 0.00479294 | 0.49 |
| IMAGE:665833 | 665833 | 0.00484015 | 0.76 |
| APLP2 | 549054 | 0.00487222 | 0.45 |
| PMS2 | 116906 | 0.00494457 | 0.54 |
| IMAGE:666946 | 666946 | 0.00542678 | 0.76 |
| SEMA7A | 135941 | 0.00543695 | 0.28 |
| IMAGE:472111 | 472111 | 0.00552796 | 0.48 |
| Transcribed | | | |
| locus | | | |
| E2F5 | 809828 | 0.00605492 | 0.36 |
| YTHDF3 | 135450 | 0.00636167 | 0.57 |
| CDC42EP2 | 26651 | 0.00640863 | 0.72 |
| PCSK5 | 2115808 | 0.0064231 | 0.54 |
| FLJ34433 | 713671 | 0.00657683 | 0.61 |
| IMAGE:743615 | 743615 | 0.00685838 | 0.72 |
| Full- | | | |
| length cDNA | | | |
| clone | | | |
| CS0DK001YE19 | | | |
| of HeLa | | | |
| cells Cot 25- | | | |
| normalized of | | | |
| Homo | | | |
| sapiens | | | |
| (human) | | | |
| IMAGE:731714 | 731714 | 0.00686952 | 0.54 |
| CDNA | | | |
| FLJ26188 fis, | | | |
| clone | | | |
| ADG04821 | | | |
| DF | 666128 | 0.00697577 | 0.45 |
| NCAM1 | 366842 | 0.00698396 | 0.61 |
| PTK2 | 724892 | 0.00728776 | 0.51 |
| PH-4 | 730938 | 0.00757078 | 0.68 |
| IMAGE:22908 | 22908 | 0.00791864 | 0.76 |
| Transcribed | | | |
| locus, weakly | | | |
| similar to | | | |
| NP_775735.1 | | | |
| I(3)mbt-like | | | |
| 4 [Homo | | | |
| sapiens] | | | |
| CCND1 | 324079 | 0.00834738 | 0.55 |
| GLG1 | 365613 | 0.00835991 | 0.55 |
| UNC13D | 231903 | 0.00842373 | 0.75 |
| IMAGE:743422 | 743422 | 0.00847541 | 0.73 |
| PSPC1 | 687541 | 0.00850591 | 0.57 |
| RBM25 | 668360 | 0.00902449 | 0.65 |
| GRM3 | 287843 | 0.00907766 | 0.63 |
| CKLFSF3 | 489249 | 0.00916924 | 0.59 |
| BCL2 | 342181 | 0.00939327 | 0.59 |
| SLC17A2 | 207920 | 0.00940866 | 0.53 |
| PRUNE | 364324 | 0.00946088 | 0.57 |
| RDH5 | 682109 | 0.00962973 | 0.63 |
| NCB5OR | 743367 | 0.00984276 | 0.42 |
| CDKN1C | 2381770 | 0.00996888 | 0.48 |
| LIPT1 | 22085 | 0.01008465 | 0.66 |
| FLJ12903 | 730123 | 0.0108591 | 0.71 |
| MORF4L1 | 135875 | 0.01141387 | 0.65 |
| UBE1L | 250883 | 0.01157076 | 0.42 |
| ADCY3 | 667061 | 0.01187515 | 0.57 |
| LOC284371 | 724126 | 0.01210721 | 0.70 |
| JAG2 | 1521706 | 0.01220002 | 0.75 |
| LPXN | 687679 | 0.01248551 | 0.62 |
| ABCC3 | 208097 | 0.01252558 | 0.49 |
| ANKRD20B | 745106 | 0.01264616 | 0.47 |
| PIAS1 | 38789 | 0.01268674 | 0.62 |
| TBC1D22A | 682479 | 0.01361669 | 0.65 |
| ZFOC1 | 744606 | 0.01461132 | 0.50 |
| IMAGE:668684 | 668684 | 0.01465001 | 0.76 |
| Transcribed | | | |
| locus, weakly | | | |
| similar to | | | |
| XP_209041.2 | | | |
| PREDICTED: | | | |
| similar to | | | |
| KIAA1503 | | | |
| protein | | | |
| [Homo | | | |
| sapiens] | | | |
| SSH3 | 726272 | 0.01468164 | 0.57 |
| KIAA0759 | 743451 | 0.01491078 | 0.72 |
| GPR108 | 25949 | 0.01515322 | 0.66 |
| RBM5 | 744001 | 0.01521305 | 0.61 |
| APOE | 1870594 | 0.01533491 | 0.39 |
| TJP1 | 179334 | 0.01571594 | 0.59 |
| FMNL2 | 364568 | 0.01590097 | 0.84 |
| CCNE1 | 357807 | 0.0161434 | 0.56 |
| MGP | 590264 | 0.01616941 | 0.33 |
| PEF | 137353 | 0.01649136 | 0.55 |
| SULT1E1 | 207926 | 0.01665512 | 0.78 |
| IMAGE:731298 | 731298 | 0.01703394 | 0.84 |
| CDNA | | | |
| FLJ42482 fis, | | | |
| clone | | | |
| BRACE2032134 | | | |
| BTBD14B | 173288 | 0.01718026 | 0.75 |
| HMGN1 | 565754 | 0.01756741 | 0.79 |
| BMP4 | 69166 | 0.01801726 | 0.58 |
| NYREN18 | 668584 | 0.01840274 | 0.46 |
| LOC92154 | 364926 | 0.01851848 | 0.85 |
| FCGBP | 154172 | 0.01854199 | 0.38 |
| IMAGE:725726 | 725726 | 0.01874028 | 0.62 |
| Hypothetical | | | |
| gene | | | |
| supported by | | | |
| AK093801 | | | |
| TETRAN | 725340 | 0.01946568 | 0.67 |
| IMAGE:23765 | 23765 | 0.01959004 | 0.59 |
| NEXN | 687625 | 0.01961234 | 0.68 |
| PHC3 | 743391 | 0.01961756 | 0.75 |
| DAXX | 292042 | 0.02012924 | 0.62 |
| LILRA1 | 2032639 | 0.02041049 | 0.50 |
| OAZ2 | 128694 | 0.02088621 | 0.67 |
| NR6A1 | 258666 | 0.0209244 | 0.71 |
| NUP214 | 382612 | 0.02101788 | 0.61 |
| CLPX | 725394 | 0.02137618 | 0.68 |
| ANKRD28 | 687381 | 0.02170849 | 0.65 |
| KDR | 469345 | 0.02183637 | 0.52 |
| MXD4 | 730036 | 0.0218509 | 0.56 |
| DCTN1 | 180640 | 0.02191199 | 0.73 |
| POU2F2 | 188393 | 0.02191557 | 0.58 |
| TOE1 | 52897 | 0.02192014 | 0.76 |
| RAD51L1 | 295412 | 0.02193486 | 0.59 |
| ESRRBL1 | 51232 | 0.02212668 | 0.59 |
| HMGB2 | 884365 | 0.02272167 | 0.86 |
| MGC4707 | 744945 | 0.02301085 | 0.66 |
| OTUB1 | 174683 | 0.02338881 | 0.55 |
| GC | 195340 | 0.02456764 | 0.71 |
| CASP7 | 279470 | 0.02489014 | 0.73 |
| NFYA | 731648 | 0.02499428 | 0.82 |
| IMAGE:665392 | 665392 | 0.02506006 | 0.73 |
| Hypothetical | | | |
| LOC388790 | | | |
| CTH | 730606 | 0.02560935 | 0.65 |
| C19orf33 | 379540 | 0.02578478 | 0.73 |
| PTK2B | 43541 | 0.0261765 | 0.68 |
| PVRL2 | 725364 | 0.02620575 | 0.71 |
| LOC340073 | 731404 | 0.0263198 | 0.51 |
| CPXM2 | 729924 | 0.0263992 | 0.75 |
| FLJ22794 | 137454 | 0.02646663 | 0.53 |
| DELGEF | 665437 | 0.02657829 | 0.78 |
| NR2F1 | 253386 | 0.02669705 | 0.57 |
| COL18A1 | 359202 | 0.02671076 | 0.51 |
| IMAGE:731685 | 731685 | 0.02686807 | 0.68 |
| Transcribed | | | |
| locus | | | |
| PP1201 | 364974 | 0.02732106 | 0.66 |
| PDLIM1 | 135689 | 0.0279473 | 0.36 |
| MAP3K4 | 727229 | 0.02928203 | 0.72 |
| ZBTB16 | 2467442 | 0.03012785 | 0.50 |
| ABCC8 | 1417901 | 0.03060287 | 0.55 |
| RASGRP1 | 725707 | 0.03127653 | 0.46 |
| FXYD1 | 204686 | 0.03137609 | 0.70 |
| RGL2 | 741891 | 0.03178792 | 0.54 |
| IMAGE:53081 | 53081 | 0.03196289 | 0.57 |
| SMAD4 | 134785 | 0.03265655 | 0.74 |
| IGF1R | 682555 | 0.03280651 | 0.65 |
| IMAGE:22384 | 22384 | 0.03316977 | 0.67 |
| CDNA | | | |
| FLJ34183 fis, | | | |
| clone | | | |
| FCBBF3016987 | | | |
| LOC286170 | 134858 | 0.03322882 | 0.62 |
| IMAGE:379279 | 379279 | 0.03411148 | 0.70 |
| FLJ31438 | 135539 | 0.0343791 | 0.68 |
| BCL2L11 | 300194 | 0.03534285 | 0.74 |
| PTHLH | 2214396 | 0.03609798 | 0.74 |
| MEF2C | 687270 | 0.03611238 | 0.66 |
| MGAT3 | 731060 | 0.03670502 | 0.61 |
| IMAGE:174861 | 174861 | 0.0368892 | 0.83 |
| Transcribed | | | |
| locus | | | |
| PSAT1 | 366388 | 0.03731675 | 0.65 |
| PDCD1LG2 | 738944 | 0.03763056 | 0.70 |
| MDC1 | 365288 | 0.03779568 | 0.73 |
| CCNL1 | 251216 | 0.03836932 | 0.63 |
| PAX8 | 545475 | 0.03869026 | 0.52 |
| CNN1 | 726779 | 0.03890173 | 0.55 |
| ACTR1B | 136868 | 0.03907238 | 0.61 |
| DKFZp434M202 | 743118 | 0.04068046 | 0.73 |
| LOC339903 | 135352 | 0.04078367 | 0.62 |
| MDGA1 | 180082 | 0.04209186 | 0.76 |
| IMAGE:2113771 | 2113771 | 0.04219577 | 0.71 |
| GALNT9 | 178750 | 0.04337842 | 0.66 |
| FLJ12604 | 731128 | 0.04447831 | 0.69 |
| ITIH5 | 179733 | 0.04468861 | 0.76 |
| CORO1B | 137205 | 0.04654503 | 0.80 |
| IMAGE:740927 | 740927 | 0.04665612 | 0.58 |
| ADAMTS9 | 376153 | 0.04687807 | 0.55 |
| IMAGE:365097 | 365097 | 0.04829692 | 0.89 |
| CDNA | | | |
| FLJ25129 fis, | | | |
| clone | | | |
| CBR06594 | | | |
| LRP1 | 30219 | 0.04992524 | 0.62 |

TABLE 22

| | OTC | Over | |
|---|---|---|---|
| name | cloid | p-value | fold |
| MGC29784 | 731019 | 6.47E−04 | 1.40 |
| KIAA1280 | 366085 | 7.06E−04 | 1.63 |
| TIMP2 | 258127 | 8.32E−04 | 1.79 |
| MAK | 382002 | 1.57E−03 | 2.09 |
| UQCRC1 | 714414 | 3.48E−03 | 1.32 |
| ALDH1A3 | 486189 | 4.84E−03 | 1.55 |
| MDH1 | 725188 | 5.29E−03 | 1.51 |
| ZNF258 | 1241974 | 1.04E−02 | 1.47 |
| OAS1 | 666703 | 1.05E−02 | 1.35 |
| LRRIQ2 | 487152 | 1.21E−02 | 2.10 |
| SDHA | 40304 | 1.53E−02 | 3.04 |
| BST2 | 811024 | 0.01594 | 1.39 |
| MESP1 | 25865 | 1.62E−02 | 1.61 |
| IMAGE:666279 | 666279 | 1.75E−02 | 1.51 |
| CDNA FLJ30779 | | | |
| fis, clone | | | |
| FEBRA2000815 | | | |
| IMAGE:745133 | 745133 | 1.90E−02 | 1.54 |
| Transcribed locus | | | |
| CXCL13 | 347362 | 2.06E−02 | 1.76 |
| RPS6KA5 | 258966 | 2.44E−02 | 1.94 |
| FBXO46 | 471664 | 2.69E−02 | 2.57 |
| HP | 82687 | 2.86E−02 | 1.37 |
| IFI30 | 740931 | 2.92E−02 | 1.80 |
| AP2S1 | 739109 | 3.03E−02 | 1.58 |
| GPD1 | 628418 | 3.09E−02 | 2.22 |
| C6orf216 | 731742 | 3.25E−02 | 1.76 |
| SAFB | 42280 | 3.28E−02 | 1.84 |
| SCMH1 | 724014 | 3.33E−02 | 1.75 |
| ABR | 1012903 | 3.39E−02 | 1.22 |
| BAK1 | 1288183 | 3.44E−02 | 2.57 |
| IMAGE:382423 | 382423 | 3.62E−02 | 3.30 |
| Transcribed locus | | | |
| COX6A1 | 512910 | 3.74E−02 | 2.21 |
| IMAGE:667527 | 667527 | 3.79E−02 | 1.39 |
| DECR2 | 730942 | 3.82E−02 | 1.10 |
| AURKC | 731021 | 4.01E−02 | 1.90 |
| IL2RA | 3054031 | 4.03E−02 | 1.75 |
| DCXR | 724596 | 4.03E−02 | 1.76 |
| CTXN1 | 179266 | 4.23E−02 | 1.18 |
| IMAGE:742061 | 742061 | 4.59E−02 | 2.62 |
| IMAGE:667683 | 667683 | 4.60E−02 | 1.93 |
| Full-length cDNA | | | |
| clone | | | |
| CS0DK008YI09 | | | |
| of HeLa cells Cot | | | |
| 25-normalized of | | | |
| Homo sapiens | | | |
| (human) | | | |
| IMAGE:743603 | 743603 | 4.68E−02 | 1.45 |
| LILRB5 | 71428 | 4.70E−02 | 1.85 |
| EIF4EBP1 | 713608 | 4.86E−02 | 1.76 |

TABLE 23

| | PTC | Under | |
|---|---|---|---|
| name | cloid | p-value | fold |
| CDH16 | 726763 | 4.1E−17 | 0.36 |
| CRABP1 | 739193 | 3E−13 | 0.16 |
| MATN2 | 28584 | 4.3E−13 | 0.29 |
| MGC99813 | 739097 | 5.6E−13 | 0.19 |
| ASGR1 | 25883 | 2.4E−11 | 0.38 |
| IMAGE:1716286 | 1716286 | 4.1E−10 | 0.37 |
| HSPC159 | 365045 | 2.2E−09 | 0.60 |
| PTPN4 | 666367 | 7.9E−09 | 0.54 |
| IRAK1 | 379200 | 1.3E−08 | 0.63 |
| GLT8D2 | 365271 | 2E−08 | 0.49 |
| MGC20781 | 731227 | 2.2E−08 | 0.47 |
| LOC440934 | 726760 | 2.3E−08 | 0.53 |
| FLJ11184 | 384404 | 2.8E−08 | 0.42 |
| KLF1 | 208991 | 2.8E−08 | 0.45 |
| FLJ14351 | 744943 | 3.2E−08 | 0.25 |
| BMP5 | 1846326 | 6.1E−08 | 0.34 |
| ZNF319 | 364729 | 7.3E−08 | 0.23 |
| OBSCN | 730926 | 7.6E−08 | 0.64 |
| FKBP6 | 795736 | 1.2E−07 | 0.45 |
| IMAGE:136686 | 136686 | 1.4E−07 | 0.59 |
| FLJ13197 | 667117 | 1.5E−07 | 0.74 |
| IMAGE:743517 | 743517 | 1.6E−07 | 0.52 |
| Transcribed | | | |
| locus | | | |
| MTAP | 724151 | 1.7E−07 | 0.55 |
| DIO1 | 296702 | 1.9E−07 | 0.32 |
| IMAGE:742837 | 742837 | 2.3E−07 | 0.65 |
| Transcribed | | | |
| locus | | | |
| PPIL4 | 364777 | 2.6E−07 | 0.29 |
| APIN | 364706 | 4.8E−07 | 0.59 |
| IMAGE:731616 | 731616 | 5.6E−07 | 0.40 |
| MRNA; | | | |
| cDNA | | | |
| DKFZp686A102 | | | |
| (from | | | |
| clone | | | |
| DKFZp686A102) | | | |
| FLJ30707 | 666986 | 6.4E−07 | 0.60 |
| VAV1 | 80384 | 6.6E−07 | 0.38 |
| USP13 | 666007 | 6.9E−07 | 0.56 |
| ABCB1 | 1837488 | 7.1E−07 | 0.48 |
| MAPRE2 | 383868 | 8.4E−07 | 0.48 |
| SDHA | 40304 | 9.2E−07 | 0.64 |
| MATN2 | 366100 | 9.4E−07 | 0.43 |
| IMAGE:744055 | 744055 | 1E−06 | 0.48 |
| Transcribed | | | |
| locus | | | |
| CCL28 | 136919 | 1.6E−06 | 0.67 |
| VTN | 230126 | 2E−06 | 0.61 |
| BAK1 | 1288183 | 2.4E−06 | 0.45 |
| TOSO | 813174 | 2.6E−06 | 0.54 |
| HBA2 | 469647 | 2.8E−06 | 0.34 |
| SLC4A4 | 787938 | 2.9E−06 | 0.46 |
| DUSP24 | 740158 | 3.3E−06 | 0.40 |
| EIF4EBP1 | 713608 | 3.8E−06 | 0.72 |
| DEPDC6 | 669318 | 4.2E−06 | 0.52 |
| SOD3 | 795309 | 4.3E−06 | 0.39 |
| TM4SF9 | 812967 | 4.9E−06 | 0.42 |
| KRTHA4 | 325155 | 5.4E−06 | 0.50 |
| GNAS | 382791 | 5.5E−06 | 0.46 |
| DCXR | 724596 | 5.5E−06 | 0.63 |
| TBCA | 743703 | 5.6E−06 | 0.63 |
| ME3 | 724238 | 6.5E−06 | 0.52 |
| FCGBP | 154172 | 8.2E−06 | 0.49 |
| GTPBP1 | 366484 | 8.4E−06 | 0.70 |
| KIF27 | 667657 | 8.4E−06 | 0.67 |
| HBB | 469549 | 8.6E−06 | 0.34 |
| SQLE | 124781 | 9.9E−06 | 0.61 |
| FLJ23577 | 668452 | 9.9E−06 | 0.43 |
| AQP11 | 713831 | 1.2E−05 | 0.58 |
| LEF1 | 713913 | 1.3E−05 | 0.65 |
| IMAGE:383718 | 383718 | 1.3E−05 | 0.60 |
| DAB1 | 382621 | 1.5E−05 | 0.58 |
| IMAGE:744616 | 744616 | 1.6E−05 | 0.63 |
| DNA | | | |
| damage | | | |
| repair and | | | |
| recombination | | | |
| protein | | | |
| RAD52 | | | |
| pseudogene | | | |
| OSR1 | 364686 | 1.7E−05 | 0.65 |
| HAS3 | 667533 | 1.7E−05 | 0.54 |
| IMAGE:744074 | 744074 | 1.8E−05 | 0.39 |
| Transcribed | | | |
| locus | | | |
| KIAA1277 | 364822 | 2E−05 | 0.66 |
| ARF4L | 49888 | 2.1E−05 | 0.59 |
| IMAGE:24587 | 24587 | 2.1E−05 | 0.59 |
| IMAGE:363955 | 363955 | 2.2E−05 | 0.51 |
| Transcribed | | | |
| locus | | | |
| PDK2 | 114008 | 2.4E−05 | 0.68 |
| PLCG2 | 201467 | 2.4E−05 | 0.63 |
| RAD51L1 | 295412 | 2.5E−05 | 0.61 |
| WNT4 | 375746 | 2.7E−05 | 0.66 |
| IMAGE:666315 | 666315 | 2.8E−05 | 0.58 |
| Homo | | | |
| sapiens, | | | |
| clone | | | |
| IMAGE:5312086, | | | |
| mRNA | | | |
| IMAGE:744385 | 744385 | 3E−05 | 0.59 |
| Similar to | | | |
| heat shock | | | |
| 10 kDa | | | |
| protein 1 | | | |
| (chaperonin | | | |
| 10); heat | | | |
| shock 10 kD | | | |
| protein 1 | | | |
| (chaperonin | | | |
| 10) | | | |
| USP4 | 544818 | 3.1E−05 | 0.43 |
| FLJ12998 | 364846 | 3.2E−05 | 0.64 |
| KLRD1 | 145696 | 3.4E−05 | 0.64 |
| IMAGE:738424 | 738424 | 3.6E−05 | 0.73 |
| MATN1 | 1624260 | 3.7E−05 | 0.43 |
| FLJ35630 | 485652 | 3.8E−05 | 0.70 |
| SPI1 | 1285305 | 3.9E−05 | 0.68 |
| IMAGE:2484270 | 2484270 | 5.3E−05 | 0.55 |
| FLJ21918 | 194302 | 6.2E−05 | 0.74 |
| CD34 | 770858 | 6.2E−05 | 0.71 |
| LOH12CR2 | 366074 | 6.9E−05 | 0.70 |
| SIAT7B | 823590 | 7.3E−05 | 0.57 |
| SCMH1 | 724014 | 7.4E−05 | 0.46 |
| IMAGE:379937 | 379937 | 7.4E−05 | 0.63 |
| TSG101 | 194350 | 7.7E−05 | 0.67 |
| GADD45G | 727321 | 7.8E−05 | 0.58 |
| C2orf7 | 366243 | 8.1E−05 | 0.72 |
| CCNE1 | 68950 | 8.2E−05 | 0.68 |
| IMAGE:364741 | 364741 | 8.4E−05 | 0.58 |
| MCC | 731305 | 8.5E−05 | 0.47 |
| PRKCABP | 1174342 | 8.8E−05 | 0.77 |
| ULK4 | 744895 | 8.9E−05 | 0.67 |
| BCL2 | 342181 | 9.2E−05 | 0.63 |
| PIP | 985457 | 0.00011 | 0.68 |
| BIN1 | 2384812 | 0.00011 | 0.67 |
| IMAGE:727289 | 727289 | 0.00012 | 0.48 |
| Transcribed | | | |
| locus | | | |
| HMG20A | 731277 | 0.00012 | 0.56 |
| COX5A | 1085884 | 0.00012 | 0.70 |
| QIL1 | 727296 | 0.00013 | 0.81 |
| IMAGE:383966 | 383966 | 0.00014 | 0.65 |
| CPXM2 | 729924 | 0.00015 | 0.58 |
| ANP32E | 382738 | 0.00016 | 0.56 |
| MRPS11 | 471574 | 0.00016 | 0.65 |
| IMAGE:682585 | 682585 | 0.00017 | 0.68 |
| ALDH3A1 | 525221 | 0.00017 | 0.64 |
| KLK2 | 1102600 | 0.00017 | 0.62 |
| C6orf85 | 665379 | 0.00017 | 0.71 |
| GNAI1 | 753215 | 0.00018 | 0.65 |
| COL18A1 | 359202 | 0.00019 | 0.62 |
| VAV2 | 1581686 | 0.00019 | 0.74 |
| SH3RF2 | 744797 | 0.00021 | 0.58 |
| IMAGE:726513 | 726513 | 0.00021 | 0.66 |
| Similar to | | | |
| 40S | | | |
| ribosomal | | | |
| protein S3 | | | |
| NME7 | 743982 | 0.00023 | 0.43 |
| LOC388284 | 731722 | 0.00023 | 0.75 |
| RASAL2 | 486304 | 0.00023 | 0.48 |
| MAPK3 | 323438 | 0.00024 | 0.64 |
| IMAGE:743422 | 743422 | 0.00024 | 0.76 |
| IMAGE:727491 | 727491 | 0.00024 | 0.41 |
| PITPNC1 | 364436 | 0.00027 | 0.69 |
| CABIN1 | 1844968 | 0.00027 | 0.67 |
| DF | 666128 | 0.00027 | 0.52 |
| GCK | 30981 | 0.00028 | 0.73 |
| SIAT8C | 382069 | 0.00028 | 0.63 |
| S100A2 | 3659591 | 0.00028 | 0.69 |
| DKFZp761P0423 | 730829 | 0.00029 | 0.65 |
| F5 | 433155 | 0.00029 | 0.55 |
| ACADSB | 687953 | 0.00029 | 0.61 |
| NEGR1 | 667176 | 0.0003 | 0.76 |
| LHB | 1671903 | 0.0003 | 0.61 |
| PRUNE | 364324 | 0.00032 | 0.64 |
| CNN1 | 726779 | 0.00037 | 0.60 |
| PHC2 | 668146 | 0.00037 | 0.62 |
| IMAGE:743579 | 743579 | 0.00038 | 0.53 |
| IMAGE:26164 | 26164 | 0.00038 | 0.76 |
| PCGF4 | 740457 | 0.0004 | 0.53 |
| RANBP10 | 307025 | 0.00042 | 0.72 |
| IMAGE:667527 | 667527 | 0.00042 | 0.54 |
| SEMA7A | 135941 | 0.00043 | 0.36 |
| LMLN | 744657 | 0.00044 | 0.68 |
| C20orf18 | 665433 | 0.00045 | 0.56 |
| PPP2R4 | 731590 | 0.00047 | 0.61 |
| IMAGE:378458 | 378458 | 0.00047 | 0.76 |
| SPON2 | 723923 | 0.0005 | 0.70 |
| PSG3 | 136747 | 0.00052 | 0.65 |
| CREB3L2 | 136399 | 0.00073 | 0.70 |
| IMAGE:744941 | 744941 | 0.00074 | 0.54 |
| LOC440728 | | | |
| IMAGE:365913 | 365913 | 0.00076 | 0.69 |
| Transcribed | | | |
| locus | | | |
| IMAGE:730924 | 730924 | 0.00078 | 0.70 |
| Transcribed | | | |
| locus | | | |
| SLC2A4RG | 376983 | 0.00081 | 0.70 |
| PRKCQ | 669149 | 0.00081 | 0.69 |
| RANBP10 | 682251 | 0.00087 | 0.68 |
| SGCG | 2046361 | 0.0009 | 0.76 |
| SYP | 30471 | 0.0009 | 0.52 |
| IMAGE:665403 | 665403 | 0.00096 | 0.82 |
| Transcribed | | | |
| locus | | | |
| TIE1 | 743043 | 0.00097 | 0.69 |
| RAB8A | 712118 | 0.00098 | 0.74 |
| CES2 | 153667 | 0.00099 | 0.76 |
| CCL15 | 342290 | 0.00099 | 0.57 |
| RNF32 | 731422 | 0.00104 | 0.47 |
| OAS1 | 666703 | 0.00104 | 0.57 |
| PKNOX1 | 667067 | 0.00108 | 0.61 |
| ATP2B4 | 665814 | 0.00116 | 0.63 |
| LOC253981 | 742743 | 0.00118 | 0.62 |
| HP | 82687 | 0.0012 | 0.78 |
| E2F5 | 809828 | 0.00127 | 0.47 |
| SEPW1 | 173628 | 0.00132 | 0.52 |
| CCR7 | 2345206 | 0.00134 | 0.68 |
| LYPLA3 | 471702 | 0.00143 | 0.75 |
| KCNQ2 | 179534 | 0.00144 | 0.62 |
| C21orf84 | 730814 | 0.00145 | 0.59 |
| CREBBP | 172996 | 0.00146 | 0.63 |
| HSPC072 | 668329 | 0.00148 | 0.72 |
| RODH | 471641 | 0.00149 | 0.54 |
| TRIP12 | 665820 | 0.00155 | 0.55 |
| MGC23909 | 731598 | 0.00155 | 0.50 |
| IMAGE:136121 | 136121 | 0.00157 | 0.71 |
| CDNA | | | |
| FLJ10247 fis, | | | |
| clone | | | |
| HEMBB1000705 | | | |
| GATA3 | 148796 | 0.00158 | 0.72 |
| SIGLEC7 | 743331 | 0.00161 | 0.74 |
| PDCD1LG1 | 1942634 | 0.00162 | 0.68 |
| IMAGE:731758 | 731758 | 0.00163 | 0.77 |
| Transcribed | | | |
| locus, weakly | | | |
| similar to | | | |
| NP_081070.1 | | | |
| kiaa-iso | | | |
| protein | | | |
| homolog; | | | |
| Band 47B | | | |
| [Mus | | | |
| musculus] | | | |
| CD38 | 1352408 | 0.00163 | 0.80 |
| ALDH1A1 | 309697 | 0.00163 | 0.55 |
| MUTED | 731202 | 0.00165 | 0.60 |
| IMAGE:472111 | 472111 | 0.00168 | 0.61 |
| Transcribed | | | |
| locus | | | |
| KLHL9 | 501527 | 0.00169 | 0.69 |
| MT1F | 78353 | 0.0017 | 0.40 |
| GPI | 741474 | 0.0017 | 0.69 |
| IMAGE:738945 | 738945 | 0.00176 | 0.79 |
| GALNT9 | 178750 | 0.0018 | 0.72 |
| IMAGE:743603 | 743603 | 0.0018 | 0.74 |
| PARD3 | 724642 | 0.00183 | 0.70 |
| KIAA0789 | 33621 | 0.00193 | 0.66 |
| AXL | 364083 | 0.00194 | 0.58 |
| TM4SF9 | 381032 | 0.00197 | 0.75 |
| ZNF76 | 745003 | 0.002 | 0.75 |
| C20orf19 | 366032 | 0.00204 | 0.71 |
| RAP1GA1 | 971276 | 0.00208 | 0.80 |
| SLC6A1 | 177967 | 0.0021 | 0.63 |
| BAZ2B | 609631 | 0.00212 | 0.78 |
| NEK6 | 725345 | 0.00212 | 0.74 |
| IMAGE:745512 | 745512 | 0.00215 | 0.74 |
| LILRB5 | 71428 | 0.00216 | 0.68 |
| GFPT2 | 485085 | 0.00217 | 0.72 |
| IMAGE:743903 | 743903 | 0.0022 | 0.60 |
| Homo | | | |
| sapiens, | | | |
| clone | | | |
| IMAGE:5266541, | | | |
| mRNA | | | |
| IMAGE:744439 | 744439 | 0.00229 | 0.71 |
| Transcribed | | | |
| locus | | | |
| BIC | 743270 | 0.00237 | 0.68 |
| IMAGE:738966 | 738966 | 0.00238 | 0.66 |
| Transcribed | | | |
| locus | | | |
| GJA5 | 196338 | 0.0024 | 0.65 |
| GRIK2 | 731363 | 0.0024 | 0.77 |
| DMD | 743394 | 0.00243 | 0.63 |
| CDR2 | 366067 | 0.00249 | 0.81 |
| IMAGE:727067 | 727067 | 0.00251 | 0.72 |
| Similar to | | | |
| hypothetical | | | |
| protein | | | |
| SB153 | | | |
| isoform 1 | | | |
| ANKRD20B | 745106 | 0.00257 | 0.61 |
| PCSK6 | 31924 | 0.00257 | 0.63 |
| DC12 | 724895 | 0.00272 | 0.74 |
| LOC388886 | 34007 | 0.00285 | 0.70 |
| IMAGE:744841 | 744841 | 0.00286 | 0.74 |
| LOC441054 | | | |
| LOC51760 | 52226 | 0.00287 | 0.77 |
| AURKC | 731021 | 0.00289 | 0.64 |
| IGSF10 | 682276 | 0.0029 | 0.82 |
| LPXN | 687679 | 0.00294 | 0.79 |
| LOC339903 | 135352 | 0.00295 | 0.72 |
| SERPINB5 | 1662274 | 0.00313 | 0.72 |
| SFRP4 | 285693 | 0.00324 | 0.57 |
| GCAT | 307094 | 0.00333 | 0.76 |
| SAFB | 42280 | 0.00336 | 0.61 |
| CTRL | 1308954 | 0.00345 | 0.81 |
| FLT4 | 668815 | 0.00347 | 0.69 |
| TTC17 | 665668 | 0.00359 | 0.75 |
| BCL2 | 232714 | 0.0036 | 0.69 |
| BMP7 | 1594414 | 0.00364 | 0.68 |
| HOXD11 | 682119 | 0.00365 | 0.59 |
| SIN3A | 26455 | 0.00377 | 0.82 |
| SIAT8F | 667110 | 0.00379 | 0.63 |
| SMAD4 | 134785 | 0.00389 | 0.79 |
| BDKRB2 | 665674 | 0.00394 | 0.65 |
| ARHGAP10 | 731380 | 0.00394 | 0.63 |
| DCTN1 | 180640 | 0.00395 | 0.73 |
| APOD | 838611 | 0.00399 | 0.43 |
| LOC440434 | 742046 | 0.00399 | 0.80 |
| LRP1 | 30219 | 0.00411 | 0.76 |
| SLC17A2 | 207920 | 0.0043 | 0.57 |
| KCNK4 | 743016 | 0.00434 | 0.70 |
| MLPH | 667259 | 0.00447 | 0.43 |
| GPD1 | 628418 | 0.00451 | 0.70 |
| FLJ25067 | 667252 | 0.00453 | 0.57 |
| PTGES | 504646 | 0.00472 | 0.72 |
| LOC286272 | 667361 | 0.00476 | 0.80 |
| IMAGE:742061 | 742061 | 0.00479 | 0.62 |
| FLJ44005 | 726675 | 0.00482 | 0.76 |
| UNC13D | 231903 | 0.00505 | 0.58 |
| DCI | 667892 | 0.00511 | 0.64 |
| SYCP2 | 136863 | 0.00522 | 0.67 |
| MGC5178 | 725533 | 0.00522 | 0.66 |
| CD33 | 1917430 | 0.00523 | 0.60 |
| PEF | 137353 | 0.00539 | 0.73 |
| KIAA1838 | 744106 | 0.00542 | 0.83 |
| SRGAP2 | 714493 | 0.00547 | 0.84 |
| FLJ22386 | 32917 | 0.00552 | 0.74 |
| MXD3 | 714013 | 0.0058 | 0.81 |
| BCL2 | 342181 | 0.00587 | 0.61 |
| TIMP3 | 501476 | 0.00587 | 0.68 |
| EIF2C4 | 730834 | 0.00636 | 0.75 |
| MAP7 | 79729 | 0.00642 | 0.76 |
| IMAGE:666794 | 666794 | 0.00646 | 0.71 |
| Hypothetical | | | |
| LOC388170 | | | |
| ZBTB16 | 2467442 | 0.00703 | 0.63 |
| NF2 | 1698236 | 0.00722 | 0.76 |
| APOL1 | 665632 | 0.00739 | 0.54 |
| DDX54 | 743268 | 0.0074 | 0.77 |
| ZNF250 | 741066 | 0.00747 | 0.48 |
| HTATIP2 | 726618 | 0.00801 | 0.83 |
| IMAGE:743290 | 743290 | 0.00804 | 0.63 |
| Transcribed | | | |
| locus | | | |
| C21orf7 | 665495 | 0.00825 | 0.78 |
| GSTM1 | 73778 | 0.00829 | 0.72 |
| IMAGE:744410 | 744410 | 0.00835 | 0.78 |
| Transcribed | | | |
| locus | | | |
| PRKCQ | 669149 | 0.00838 | 0.70 |
| KIAA1463 | 381118 | 0.00846 | 0.73 |
| ETV4 | 809959 | 0.00848 | 0.77 |
| IGHM | 276658 | 0.00848 | 0.79 |
| COX6B1 | 632026 | 0.00854 | 0.72 |
| RUNX3 | 122874 | 0.00873 | 0.74 |
| PSCD3 | 744050 | 0.00895 | 0.75 |
| C6orf118 | 731745 | 0.00902 | 0.74 |
| PLCG1 | 1174287 | 0.00935 | 0.79 |
| IMAGE:745123 | 745123 | 0.00936 | 0.51 |
| Transcribed | | | |
| locus | | | |
| MRC2 | 342581 | 0.00949 | 0.58 |
| C4orf16 | 681890 | 0.00979 | 0.78 |
| ANAPC2 | 136462 | 0.00996 | 0.79 |
| CDC42EP2 | 26651 | 0.00999 | 0.84 |
| DEXI | 668186 | 0.0102 | 0.79 |
| ATP5G3 | 740090 | 0.0105 | 0.86 |
| AKR1C1 | 196992 | 0.01063 | 0.31 |
| CBL | 1578721 | 0.0107 | 0.71 |
| IMAGE:745072 | 745072 | 0.01074 | 0.61 |
| Transcribed | | | |
| locus | | | |
| SGSH | 669263 | 0.0108 | 0.78 |
| PINK1 | 729929 | 0.01102 | 0.76 |
| MFAP3L | 726821 | 0.01112 | 0.75 |
| C10orf116 | 740941 | 0.01117 | 0.73 |
| LILRB3 | 1422194 | 0.01144 | 0.79 |
| UQCRC1 | 714414 | 0.01184 | 0.80 |
| CaMKIINalpha | 173820 | 0.01261 | 0.81 |
| TMED4 | 251250 | 0.01265 | 0.74 |
| KIAA1754L | 365056 | 0.01278 | 0.71 |
| PLXNC1 | 724609 | 0.01313 | 0.80 |
| TFF1 | 1075949 | 0.01318 | 0.59 |
| COX6A1 | 512910 | 0.01348 | 0.74 |
| AMD1 | 132752 | 0.01358 | 0.72 |
| IMAGE:740105 | 740105 | 0.01359 | 0.83 |
| NTRK2 | 2048801 | 0.01375 | 0.59 |
| RPL27A | 178255 | 0.01408 | 0.64 |
| MRPL45 | 364717 | 0.01449 | 0.71 |
| ANKRD28 | 687379 | 0.0149 | 0.58 |
| DCAMKL1 | 232388 | 0.01532 | 0.73 |
| BCL2 | 342181 | 0.0154 | 0.72 |
| FBXL16 | 178908 | 0.01584 | 0.86 |
| IMAGE:744604 | 744604 | 0.01636 | 0.76 |
| TRIM8 | 714498 | 0.01646 | 0.75 |
| IMAGE:665342 | 665342 | 0.01671 | 0.83 |
| Transcribed | | | |
| locus, | | | |
| strongly | | | |
| similar to | | | |
| NP_065847.1 | | | |
| semaphorin | | | |
| 6A1; | | | |
| semaphorin | | | |
| 6A-1 [Homo | | | |
| sapiens] | | | |
| MPST | 731241 | 0.01715 | 0.69 |
| LRRIQ2 | 487152 | 0.01726 | 0.77 |
| NK4 | 504158 | 0.01782 | 0.76 |
| FLJ37587 | 668476 | 0.01845 | 0.86 |
| IMAGE:743619 | 743619 | 0.01869 | 0.78 |
| THAP3 | 687800 | 0.01904 | 0.59 |
| FSHPRH1 | 667355 | 0.01935 | 0.78 |
| ADAM12 | 724812 | 0.01958 | 0.73 |
| SIAT8B | 33133 | 0.01996 | 0.77 |
| H17 | 739432 | 0.01996 | 0.80 |
| PH-4 | 730938 | 0.02017 | 0.78 |
| MGP | 590264 | 0.02027 | 0.65 |
| BBS4 | 726634 | 0.02117 | 0.86 |
| FBXW8 | 687532 | 0.02149 | 0.82 |
| FRZB | 146049 | 0.02151 | 0.71 |
| IMAGE:213529 | 213529 | 0.02296 | 0.83 |
| Transcribed | | | |
| locus | | | |
| IMAGE:687896 | 687896 | 0.02327 | 0.83 |
| PREDICTED: | | | |
| Homo | | | |
| sapiens | | | |
| olfactory | | | |
| receptor, | | | |
| family 7, | | | |
| subfamily E, | | | |
| member 31 | | | |
| pseudogene | | | |
| (OR7E31P), | | | |
| mRNA | | | |
| SLAMF1 | 1626951 | 0.0241 | 0.82 |
| ABCA8 | 284828 | 0.0243 | 0.70 |
| CLGN | 1049033 | 0.02437 | 0.81 |
| CASP5 | 341763 | 0.02446 | 0.63 |
| SYN3 | 727056 | 0.02495 | 0.57 |
| IL2RA | 3054031 | 0.02508 | 0.75 |
| NME1 | 726600 | 0.02562 | 0.81 |
| ICAM3 | 156183 | 0.02566 | 0.83 |
| SLC36A3 | 731115 | 0.02573 | 0.83 |
| KLHL9 | 471696 | 0.0258 | 0.61 |
| CKLFSF4 | 143759 | 0.02637 | 0.81 |
| PLAC9 | 366115 | 0.02652 | 0.74 |
| IMAGE:383528 | 383528 | 0.02704 | 0.36 |
| Transcribed | | | |
| locus, weakly | | | |
| similar to | | | |
| XP_375099.1 | | | |
| PREDICTED: | | | |
| similar to | | | |
| hypothetical | | | |
| protein | | | |
| FLJ25224 | | | |
| [Homo | | | |
| sapiens] | | | |
| C6orf216 | 731742 | 0.03001 | 0.82 |
| SASH1 | 31120 | 0.0303 | 0.77 |
| IMAGE:174861 | 174861 | 0.03087 | 0.85 |
| Transcribed | | | |
| locus | | | |
| NYREN18 | 668584 | 0.0313 | 0.77 |
| KIAA0759 | 743451 | 0.03159 | 0.55 |
| SLC9A5 | 112819 | 0.0328 | 0.83 |
| JAK1 | 2030501 | 0.03295 | 0.77 |
| IMAGE:724556 | 724556 | 0.03315 | 0.77 |
| Transcribed | | | |
| locus | | | |
| IL11RA | 1101773 | 0.03445 | 0.75 |
| IL6ST | 137010 | 0.03464 | 0.82 |
| CCR7 | 2345206 | 0.03474 | 0.79 |
| FOXN4 | 731076 | 0.03623 | 0.80 |
| SPTLC2 | 365000 | 0.03624 | 0.83 |
| CCL19 | 1707527 | 0.03653 | 0.38 |
| DGKQ | 366233 | 0.03722 | 0.83 |
| SENP6 | 366436 | 0.0378 | 0.85 |
| ID1 | 1087348 | 0.04088 | 0.71 |
| RBM25 | 668360 | 0.04092 | 0.83 |
| CASP10 | 241481 | 0.04117 | 0.72 |
| LOC340073 | 731404 | 0.04151 | 0.81 |
| PTDSS2 | 21716 | 0.04197 | 0.84 |
| KIAA1196 | 738938 | 0.04253 | 0.74 |
| KCNC3 | 53333 | 0.04428 | 0.82 |
| PSMD11 | 383945 | 0.04455 | 0.70 |
| LRP2 | 2055272 | 0.04533 | 0.82 |
| EBAG9 | 682507 | 0.04536 | 0.78 |
| BAP1 | 1012990 | 0.04607 | 0.70 |
| GIMAP5 | 180259 | 0.04611 | 0.80 |
| MMRN1 | 759865 | 0.04613 | 0.81 |
| SAP30L | 723950 | 0.0485 | 0.85 |

TABLE 24

| | PTC | Over | |
|---|---|---|---|
| name | cloid | p-value | fold |
| HIVEP1 | 758037 | 1.36E−04 | 1.49 |
| IL19 | 1932168 | 1.39E−04 | 1.87 |
| CTTN | 489945 | 1.62E−04 | 1.64 |
| DJ462O23.2 | 738970 | 1.69E−04 | 1.55 |
| TRAF4 | 667756 | 1.72E−04 | 1.47 |
| CITED1 | 265558 | 2.13E−04 | 4.50 |
| IMAGE:2113771 | 2113771 | 2.84E−04 | 1.61 |
| IMAGE:365562 | 365562 | 3.36E−04 | 1.60 |
| Transcribed | | | |
| locus, | | | |
| moderately | | | |
| similar to | | | |
| XP_521389.1 | | | |
| PREDICTED: | | | |
| similar to | | | |
| UTY [Pan | | | |
| troglodytes] | | | |
| ZNF217 | 1712992 | 3.47E−04 | 1.58 |
| SENP6 | 739237 | 3.79E−04 | 1.71 |
| FLJ31438 | 135539 | 4.65E−04 | 1.52 |
| IMAGE:159809 | 159809 | 4.96E−04 | 1.28 |
| CTNNA1 | 268972 | 5.75E−04 | 1.31 |
| HT007 | 135303 | 6.22E−04 | 1.52 |
| STAT6 | 1916307 | 6.41E−04 | 1.36 |
| CD5 | 356841 | 6.57E−04 | 3.22 |
| LOC126669 | 682088 | 6.73E−04 | 1.83 |
| DELGEF | 665437 | 6.79E−04 | 1.30 |
| MGC4707 | 744945 | 7.09E−04 | 1.49 |
| KLRD1 | 2728204 | 7.15E−04 | 2.45 |
| CCND1 | 324079 | 7.23E−04 | 2.88 |
| IMAGE:727540 | 727540 | 7.97E−04 | 1.92 |
| CTTN | 470408 | 8.04E−04 | 1.65 |
| PREB | 740347 | 8.06E−04 | 5.33 |
| FMO5 | 364526 | 8.18E−04 | 2.22 |
| DPP4 | 343987 | 8.52E−04 | 5.49 |
| SPATA12 | 730300 | 8.67E−04 | 1.54 |
| CLDN1 | 594279 | 9.22E−04 | 10.32 |
| TIMP1 | 162246 | 9.38E−04 | 4.99 |
| APLP2 | 549054 | 1.03E−03 | 1.86 |
| IMP-2 | 743774 | 1.25E−03 | 2.09 |
| GRB7 | 510318 | 1.32E−03 | 1.47 |
| IMAGE:136976 | 136976 | 1.34E−03 | 2.61 |
| Transcribed | | | |
| locus, | | | |
| moderately | | | |
| similar to | | | |
| NP_777603.1 | | | |
| hypothetical | | | |
| protein | | | |
| FLJ25976 | | | |
| [Homo | | | |
| sapiens] | | | |
| PCTK3 | 725677 | 1.35E−03 | 1.41 |
| PARP9 | 667440 | 1.37E−03 | 1.54 |
| CAPN3 | 757248 | 1.49E−03 | 1.74 |
| GSS | 140405 | 1.51E−03 | 2.29 |
| PTHLH | 2214396 | 1.73E−03 | 1.86 |
| MPO | 436554 | 1.87E−03 | 1.30 |
| CCNE1 | 357807 | 1.89E−03 | 1.69 |
| STAT2 | 2306096 | 1.92E−03 | 1.58 |
| SCEL | 668239 | 1.95E−03 | 2.05 |
| STAU | 365919 | 2.10E−03 | 1.37 |
| ATIC | 665693 | 0.00217 | 1.63 |
| CD63 | 125552 | 2.40E−03 | 1.58 |
| PITPNA | 725585 | 2.54E−03 | 3.56 |
| CASP3 | 823680 | 2.82E−03 | 2.49 |
| CDH3 | 359051 | 2.87E−03 | 5.54 |
| BMP5 | 1846326 | 2.94E−03 | 1.85 |
| MDK | 309009 | 3.00E−03 | 3.16 |
| LOC120224 | 252291 | 3.05E−03 | 2.20 |
| IMAGE:1877668 | 1877668 | 0.00311 | 1.70 |
| IL13 | 1505308 | 3.26E−03 | 1.57 |
| TARP | 145105 | 3.35E−03 | 3.00 |
| MAPK14 | 1741589 | 3.45E−03 | 1.49 |
| CCND3 | 327182 | 3.54E−03 | 1.69 |
| POU2F2 | 188393 | 3.59E−03 | 2.76 |
| PET112L | 743125 | 3.61E−03 | 2.23 |
| IMAGE:132933 | 132933 | 3.64E−03 | 1.59 |
| TOE1 | 52897 | 3.70E−03 | 1.27 |
| IMAGE:731751 | 731751 | 3.81E−03 | 1.79 |
| Transcribed | | | |
| locus | | | |
| NOTCH1 | 359461 | 3.88E−03 | 2.14 |
| CACNB2 | 173841 | 3.90E−03 | 2.97 |
| ABCC5 | 212366 | 0.00419 | 1.83 |
| DNMT1 | 768241 | 4.26E−03 | 1.60 |
| YWHAH | 324066 | 5.16E−03 | 1.31 |
| SMARCA5 | 730037 | 5.18E−03 | 1.46 |
| MRC2 | 235882 | 5.27E−03 | 4.72 |
| IMAGE:668300 | 668300 | 5.32E−03 | 1.38 |
| Transcribed | | | |
| locus, | | | |
| strongly | | | |
| similar to | | | |
| NP_683708.1 | | | |
| Toll- | | | |
| interleukin 1 | | | |
| receptor | | | |
| domain- | | | |
| containing | | | |
| adaptor | | | |
| protein | | | |
| isoform b; | | | |
| adapter | | | |
| protein wyatt; | | | |
| MyD88 | | | |
| adapter-like | | | |
| protein; TIR | | | |
| domain- | | | |
| containing | | | |
| adapter | | | |
| protein; Toll- | | | |
| interleukin 1 | | | |
| receptor | | | |
| (TIR) | | | |
| domain- | | | |
| containing | | | |
| adaptor | | | |
| protein | | | |
| [Homo | | | |
| sapiens] | | | |
| IMAGE:2266583 | 2266583 | 5.45E−03 | 1.63 |
| HOXB6 | 738358 | 5.48E−03 | 1.52 |
| SPINT1 | 723914 | 5.81E−03 | 1.60 |
| PMS2 | 116906 | 5.86E−03 | 1.67 |
| RFX2 | 731738 | 6.21E−03 | 2.41 |
| GABRE | 209137 | 6.28E−03 | 1.45 |
| TSC | 745490 | 6.58E−03 | 5.91 |
| ABCC3 | 208097 | 6.60E−03 | 4.33 |
| VCL | 44193 | 6.73E−03 | 1.39 |
| CD8B1 | 1743279 | 6.94E−03 | 1.28 |
| PPP3CC | 110481 | 7.14E−03 | 1.79 |
| ETV1 | 24541 | 7.26E−03 | 1.37 |
| CD9 | 727251 | 7.34E−03 | 1.65 |
| USP18 | 745083 | 7.42E−03 | 2.07 |
| LAMB3 | 1103402 | 7.43E−03 | 1.93 |
| ETHE1 | 681957 | 7.45E−03 | 1.54 |
| PDLIM1 | 135689 | 7.58E−03 | 1.59 |
| CD59 | 132935 | 7.88E−03 | 1.35 |
| FLJ12604 | 731128 | 8.59E−03 | 2.31 |
| ADAMTS9 | 376153 | 9.32E−03 | 2.27 |
| CA11 | 282587 | 9.49E−03 | 1.61 |
| PGR1 | 666361 | 9.68E−03 | 1.22 |
| MORF4L1 | 135875 | 9.72E−03 | 1.46 |
| IBRDC3 | 726636 | 1.00E−02 | 1.29 |
| NOTCH2 | 1641901 | 1.01E−02 | 2.81 |
| ETV4 | 1690788 | 1.03E−02 | 1.60 |
| MAPK14 | 2011082 | 1.06E−02 | 1.51 |
| RNF123 | 366159 | 1.07E−02 | 1.39 |
| COX15 | 32784 | 1.09E−02 | 1.56 |
| RPS6KA2 | 22711 | 1.10E−02 | 1.50 |
| FLJ34433 | 713671 | 1.14E−02 | 1.42 |
| TU3A | 44881 | 1.17E−02 | 2.03 |
| IL13RA1 | 1492440 | 1.19E−02 | 1.31 |
| IKBKE | 364448 | 1.21E−02 | 1.41 |
| CD47 | 365526 | 1.22E−02 | 1.66 |
| RASGRP1 | 725707 | 0.01241 | 4.66 |
| CKS2 | 725454 | 1.25E−02 | 1.55 |
| FUS | 365348 | 1.28E−02 | 1.35 |
| HDAC1 | 1896337 | 1.29E−02 | 1.87 |
| IMAGE:251427 | 251427 | 1.30E−02 | 2.25 |
| KRT18 | 725096 | 1.32E−02 | 1.55 |
| FLJ22794 | 137454 | 1.38E−02 | 1.53 |
| RYK | 727092 | 1.41E−02 | 1.26 |
| CD47 | 357442 | 1.53E−02 | 1.81 |
| GTPBP5 | 725502 | 1.53E−02 | 3.08 |
| CAPNS1 | 152655 | 1.59E−02 | 1.40 |
| WHSC2 | 668283 | 1.60E−02 | 1.47 |
| IMAGE:136801 | 136801 | 1.66E−02 | 1.25 |
| PTK2B | 43541 | 1.70E−02 | 1.81 |
| KPNA2 | 667727 | 1.82E−02 | 1.31 |
| BCAP29 | 725970 | 1.88E−02 | 3.23 |
| MLANA | 266361 | 1.88E−02 | 1.41 |
| BZRP | 135991 | 1.90E−02 | 1.31 |
| SAMD10 | 365801 | 0.02007 | 1.25 |
| NR2F6 | 1238492 | 2.03E−02 | 1.29 |
| ABCC8 | 1417901 | 2.11E−02 | 1.36 |
| LEMD1 | 731047 | 2.12E−02 | 2.25 |
| WDR20 | 665156 | 2.14E−02 | 1.61 |
| S100A1 | 175772 | 2.22E−02 | 1.84 |
| CD4 | 141216 | 2.36E−02 | 1.41 |
| EMILIN2 | 365521 | 2.39E−02 | 1.20 |
| IMAGE:742919 | 742919 | 2.43E−02 | 1.37 |
| Transcribed | | | |
| locus | | | |
| NCB5OR | 743367 | 2.53E−02 | 2.21 |
| IMAGE:731299 | 731299 | 2.65E−02 | 1.43 |
| INHBB | 730012 | 2.67E−02 | 3.67 |
| CKLFSF3 | 489249 | 2.69E−02 | 1.31 |
| MLL4 | 744980 | 2.78E−02 | 1.38 |
| ECM1 | 301122 | 2.79E−02 | 16.96 |
| TM4SF8 | 713647 | 0.02811 | 1.52 |
| NFIB | 416959 | 2.88E−02 | 1.52 |
| TIAM1 | 23612 | 2.96E−02 | 3.87 |
| BID | 128065 | 2.99E−02 | 1.76 |
| SPINL | 236399 | 3.00E−02 | 3.76 |
| CLDN7 | 726335 | 3.31E−02 | 1.41 |
| PSAT1 | 366388 | 3.42E−02 | 1.38 |
| IMAGE:731298 | 731298 | 3.47E−02 | 1.37 |
| CDNA | | | |
| FLJ42482 fis, | | | |
| clone | | | |
| BRACE2032134 | | | |
| IMAGE:384087 | 384087 | 3.53E−02 | 1.88 |
| CLDN7 | 300268 | 3.57E−02 | 1.40 |
| KCNK5 | 134978 | 3.62E−02 | 2.15 |
| CASP7 | 279470 | 0.03652 | 1.50 |
| LOC389906 | 740718 | 3.72E−02 | 1.20 |
| HCLS1 | 665452 | 3.93E−02 | 1.56 |
| PRDX2 | 208439 | 4.00E−02 | 1.31 |
| IMAGE:738332 | 738332 | 4.07E−02 | 2.68 |
| Transcribed | | | |
| locus | | | |
| SSH3 | 726272 | 4.08E−02 | 1.26 |
| IMAGE:136014 | 136014 | 4.20E−02 | 1.72 |
| EGFR | 135980 | 4.21E−02 | 1.72 |
| MGAT3 | 731060 | 4.24E−02 | 1.61 |
| LGALS3BP | 742100 | 4.31E−02 | 1.57 |
| NSUN4 | 364570 | 4.32E−02 | 1.50 |
| NOG | 487474 | 4.37E−02 | 1.75 |
| S100A4 | 868577 | 4.54E−02 | 3.81 |
| GADD45GIP1 | 1084386 | 4.67E−02 | 1.25 |
| DHRS6 | 364412 | 4.88E−02 | 1.30 |
| PTPN4 | 249311 | 0.04967 | 1.49 |

TABLE 25

| | AT | Under | |
|---|---|---|---|
| name | cloid | p-value | fold |
| FCGBP | 154172 | 1.00E−07 | 0.08 |
| MT1F | 78353 | 1.00E−07 | 0.17 |
| SEMA7A | 135941 | 1.00E−07 | 0.23 |
| PAX8 | 545475 | 1.00E−07 | 0.24 |
| ID1 | 1087348 | 1.00E−07 | 0.25 |
| IMAGE:744055 | 744055 | 1.00E−07 | 0.27 |
| LOC400451 | 667174 | 1.00E−07 | 0.29 |
| KRT7 | 592276 | 1.00E−07 | 0.30 |
| DSP | 135975 | 1.00E−07 | 0.31 |
| CDH1 | 214008 | 1.00E−07 | 0.32 |
| RAD51L1 | 295412 | 1.00E−07 | 0.32 |
| APLP2 | 549054 | 1.00E−07 | 0.33 |
| GNB4 | 813260 | 1.00E−07 | 0.33 |
| TSHR | 565317 | 1.00E−07 | 0.34 |
| SLC4A4 | 787938 | 1.00E−07 | 0.35 |
| FGFRL1 | 739956 | 1.00E−07 | 0.36 |
| S100A13 | 1104732 | 1.00E−07 | 0.36 |
| CDS1 | 135630 | 1.00E−07 | 0.37 |
| DCI | 667892 | 1.00E−07 | 0.37 |
| KRT18 | 725096 | 1.00E−07 | 0.39 |
| NR2F2 | 72744 | 1.00E−07 | 0.40 |
| DNMT3B | 645455 | 1.00E−07 | 0.40 |
| IMAGE:744439 | 744439 | 1.00E−07 | 0.43 |
| CKLFSF4 | 143759 | 1.00E−07 | 0.43 |
| ATP8B1 | 669574 | 1.00E−07 | 0.43 |
| IMAGE:136686 | 136686 | 1.00E−07 | 0.44 |
| IMAGE:2266583 | 2266583 | 1.00E−07 | 0.44 |
| ICOSLG | 2074228 | 1.00E−07 | 0.44 |
| DHRS6 | 364412 | 1.00E−07 | 0.44 |
| CHRAC1 | 724276 | 1.00E−07 | 0.46 |
| GFM2 | 364959 | 1.00E−07 | 0.47 |
| IQCA | 669510 | 1.00E−07 | 0.47 |
| PEMT | 742580 | 1.00E−07 | 0.49 |
| CD9 | 727251 | 1.00E−07 | 0.50 |
| CLDN7 | 300268 | 1.00E−07 | 0.50 |
| MGC17299 | 713422 | 1.00E−07 | 0.55 |
| IMAGE:724416 | 724416 | 1.00E−04 | 0.35 |
| SASH1 | 31120 | 1.00E−04 | 0.43 |
| GOLPH2 | 811582 | 1.00E−04 | 0.45 |
| GRB10 | 564994 | 1.00E−04 | 0.47 |
| IMAGE:1877668 | 1877668 | 1.00E−04 | 0.48 |
| MATN2 | 28584 | 2.00E−04 | 0.32 |
| LRP2 | 2055272 | 3.00E−04 | 0.42 |
| POLI | 137720 | 3.00E−04 | 0.42 |
| NFKB1 | 687782 | 3.00E−04 | 0.45 |
| GNAI1 | 753215 | 3.00E−04 | 0.49 |
| C6orf176 | 173200 | 3.00E−04 | 0.55 |
| FARP1 | 486708 | 4.00E−04 | 0.49 |
| IMAGE:1716286 | 1716286 | 6.00E−04 | 0.36 |
| VEGFC | 503189 | 6.00E−04 | 0.52 |
| GJA1 | 839101 | 8.00E−04 | 0.47 |
| RODH | 471641 | 1.30E−03 | 0.25 |
| ECRG4 | 472114 | 1.50E−03 | 0.35 |
| FLJ10748 | 726830 | 1.50E−03 | 0.54 |
| PH-4 | 730938 | 1.50E−03 | 0.60 |
| SH3RF2 | 744797 | 1.60E−03 | 0.51 |
| NIFIE14 | 472160 | 1.70E−03 | 0.53 |
| E2F5 | 809828 | 2.00E−03 | 0.38 |
| ZBTB16 | 2467442 | 2.00E−03 | 0.41 |
| DEXI | 668186 | 2.00E−03 | 0.62 |
| DEPDC6 | 669318 | 2.10E−03 | 0.51 |
| DJ462O23.2 | 738970 | 2.50E−03 | 0.61 |
| CCND1 | 324079 | 3.20E−03 | 0.47 |
| KIAA0789 | 33621 | 3.60E−03 | 0.45 |
| APIN | 364706 | 4.10E−03 | 0.52 |
| LOC126669 | 682088 | 4.10E−03 | 0.57 |
| RHOD | 591907 | 4.20E−03 | 0.59 |
| ZFOC1 | 744606 | 4.40E−03 | 0.58 |
| IMAGE:738332 | 738332 | 4.70E−03 | 0.36 |
| ESRRBL1 | 51232 | 4.80E−03 | 0.58 |
| HSPB3 | 502598 | 5.30E−03 | 0.41 |
| CRABP1 | 739193 | 5.40E−03 | 0.22 |
| SLC25A23 | 741954 | 5.40E−03 | 0.52 |
| IMAGE:744616 | 744616 | 5.40E−03 | 0.61 |
| DCXR | 724596 | 5.50E−03 | 0.63 |
| NDUFA7 | 364469 | 6.10E−03 | 0.62 |
| BIN1 | 2384812 | 6.60E−03 | 0.62 |
| LOC388886 | 34007 | 6.80E−03 | 0.57 |
| MGC99813 | 739097 | 7.50E−03 | 0.30 |
| LATS2 | 666778 | 7.50E−03 | 0.59 |
| ESHPRH1 | 667355 | 7.60E−03 | 0.61 |
| TM4SF8 | 713647 | 7.70E−03 | 0.62 |
| LRRC28 | 26519 | 8.20E−03 | 0.54 |
| LOC340073 | 731404 | 8.40E−03 | 0.57 |
| PDCD4 | 665376 | 9.40E−03 | 0.52 |
| ANKRD20B | 745106 | 1.05E−02 | 0.54 |
| IMAGE:744385 | 744385 | 1.26E−02 | 0.57 |
| MESDC2 | 713653 | 1.29E−02 | 0.65 |
| USP13 | 666007 | 1.36E−02 | 0.60 |
| PVRL2 | 725364 | 1.43E−02 | 0.65 |
| CREB3L2 | 136399 | 1.47E−02 | 0.52 |
| ZNF319 | 364729 | 1.71E−02 | 0.36 |
| HSPB1 | 23827 | 1.71E−02 | 0.60 |
| RBBP4 | 705147 | 1.79E−02 | 0.67 |
| FLJ23577 | 668452 | 1.82E−02 | 0.49 |
| SGSH | 669263 | 1.84E−02 | 0.66 |
| KLF1 | 208991 | 1.91E−02 | 0.50 |
| LARS | 135407 | 1.93E−02 | 0.67 |
| LOC285961 | 742542 | 2.01E−02 | 0.69 |
| IMAGE:669188 | 669188 | 2.04E−02 | 0.56 |
| PDLIM1 | 135689 | 2.06E−02 | 0.56 |
| KIAA1196 | 738938 | 2.17E−02 | 0.64 |
| COX6A1 | 512910 | 2.19E−02 | 0.65 |
| MRPS11 | 471574 | 2.38E−02 | 0.61 |
| RPS6KA2 | 22711 | 2.50E−02 | 0.58 |
| DNALI1 | 782688 | 2.52E−02 | 0.57 |
| DKFZp434M202 | 743118 | 2.65E−02 | 0.67 |
| MFAP3L | 726821 | 2.80E−02 | 0.70 |
| PPIL4 | 364777 | 2.95E−02 | 0.63 |
| HSPC159 | 365045 | 2.96E−02 | 0.65 |
| C10orf116 | 740941 | 2.97E−02 | 0.51 |
| PLA2G6 | 744087 | 3.00E−02 | 0.61 |
| PRUNE | 364324 | 3.36E−02 | 0.67 |
| ALDH1A1 | 309697 | 3.43E−02 | 0.54 |
| DKFZp761P0423 | 730829 | 3.49E−02 | 0.59 |
| IMAGE:665649 | 665649 | 3.64E−02 | 0.67 |
| IMAGE:136014 | 136014 | 3.74E−02 | 0.62 |
| FLT4 | 668815 | 4.10E−02 | 0.70 |
| NOTCH1 | 359461 | 4.18E−02 | 0.68 |
| C20orf19 | 366032 | 4.39E−02 | 0.71 |
| IMAGE:726782 | 726782 | 4.57E−02 | 0.68 |
| NAT5 | 731073 | 4.89E−02 | 0.69 |
| ABCB1 | 1837488 | 2.26E−01 | 0.72 |
| ABCB8 | 238705 | 3.51E−01 | 0.84 |

TABLE 26

| | AT | Over | |
|---|---|---|---|
| name | cloid | p-value | fold |
| IMAGE:665833 | 665833 | 1.00E−07 | 1.95 |
| PDK1 | 1645668 | 1.00E−07 | 2.09 |
| MGAT3 | 731060 | 1.00E−07 | 2.14 |
| S100A12 | 1705397 | 1.00E−07 | 2.14 |
| IMAGE:741835 | 741835 | 1.00E−07 | 2.15 |
| BAZ2B | 609631 | 1.00E−07 | 2.16 |
| IMAGE:2113771 | 2113771 | 1.00E−07 | 2.28 |
| ITGB7 | 1337232 | 1.00E−07 | 2.31 |
| IFITM3 | 713623 | 1.00E−07 | 2.33 |
| NTRK3 | 35356 | 1.00E−07 | 2.41 |
| KIAA1737 | 122063 | 1.00E−07 | 2.41 |
| ARVCF | 40633 | 1.00E−07 | 2.47 |
| FLRT3 | 812143 | 1.00E−07 | 2.48 |
| BST2 | 811024 | 1.00E−07 | 2.48 |
| ANXA11 | 137238 | 1.00E−07 | 2.55 |
| CD1C | 665881 | 1.00E−07 | 2.58 |
| GSS | 140405 | 1.00E−07 | 2.58 |
| CD3D | 377560 | 1.00E−07 | 2.61 |
| AKT1 | 740193 | 1.00E−07 | 2.61 |
| IMAGE:727521 | 727521 | 1.00E−07 | 2.62 |
| IMAGE:731007 | 731007 | 1.00E−07 | 2.62 |
| ISG20 | 740604 | 1.00E−07 | 2.69 |
| LAIR1 | 665566 | 1.00E−07 | 2.70 |
| MGC35194 | 731098 | 1.00E−07 | 2.71 |
| UBE4B | 285666 | 1.00E−07 | 2.74 |
| TACC3 | 255398 | 1.00E−07 | 2.76 |
| WDR5 | 731023 | 1.00E−07 | 2.77 |
| TRAF5 | 1286238 | 1.00E−07 | 2.78 |
| GADD45GIP1 | 1084386 | 1.00E−07 | 2.79 |
| SMARCA5 | 730037 | 1.00E−07 | 2.79 |
| FCHO1 | 742763 | 1.00E−07 | 2.79 |
| LILRB2 | 202897 | 1.00E−07 | 2.81 |
| ATP6V1G2 | 726424 | 1.00E−07 | 2.82 |
| CHTF18 | 740971 | 1.00E−07 | 2.82 |
| TNFRSF1B | 73703 | 1.00E−07 | 2.88 |
| ENPP2 | 120916 | 1.00E−07 | 2.88 |
| AMICA1 | 487436 | 1.00E−07 | 2.92 |
| MGC4368 | 730968 | 1.00E−07 | 2.93 |
| CHRD | 471947 | 1.00E−07 | 2.94 |
| VDR | 324140 | 1.00E−07 | 2.96 |
| IMAGE:730032 | 730032 | 1.00E−07 | 2.97 |
| ARID3A | 666510 | 1.00E−07 | 2.99 |
| CASP4 | 356960 | 1.00E−07 | 3.00 |
| IFNAR2 | 123950 | 1.00E−07 | 3.05 |
| RGS19 | 485803 | 1.00E−07 | 3.05 |
| PSMB10 | 308564 | 1.00E−07 | 3.07 |
| ACE2 | 730971 | 1.00E−07 | 3.09 |
| S100A8 | 1089513 | 1.00E−07 | 3.10 |
| CD3G | 110938 | 1.00E−07 | 3.12 |
| PLCG2 | 201467 | 1.00E−07 | 3.12 |
| IMAGE:727554 | 727554 | 1.00E−07 | 3.13 |
| CXorf9 | 669503 | 1.00E−07 | 3.15 |
| IMAGE:730413 | 730413 | 1.00E−07 | 3.18 |
| MGC8407 | 179857 | 1.00E−07 | 3.21 |
| CD3Z | 1899942 | 1.00E−07 | 3.21 |
| CD69 | 276727 | 1.00E−07 | 3.22 |
| POU2F2 | 188393 | 1.00E−07 | 3.23 |
| ITGAX | 234357 | 1.00E−07 | 3.27 |
| CDX2 | 566585 | 1.00E−07 | 3.28 |
| MDM2 | 239951 | 1.00E−07 | 3.28 |
| ABCB9 | 36371 | 1.00E−07 | 3.28 |
| ICAM3 | 156183 | 1.00E−07 | 3.28 |
| C13orf12 | 23121 | 1.00E−07 | 3.32 |
| VCAM1 | 471101 | 1.00E−07 | 3.33 |
| ABCF2 | 1086914 | 1.00E−07 | 3.37 |
| IMAGE:21496 | 21496 | 1.00E−07 | 3.39 |
| GIMAP5 | 180259 | 1.00E−07 | 3.39 |
| IMAGE:727173 | 727173 | 1.00E−07 | 3.40 |
| B2M | 342416 | 1.00E−07 | 3.40 |
| FABP5 | 1088781 | 1.00E−07 | 3.44 |
| HCA112 | 486201 | 1.00E−07 | 3.46 |
| HCG4P6 | 325008 | 1.00E−07 | 3.48 |
| TOSO | 813174 | 1.00E−07 | 3.49 |
| VPREB3 | 2349125 | 1.00E−07 | 3.50 |
| HA-1 | 484504 | 1.00E−07 | 3.56 |
| SELPLG | 2366175 | 1.00E−07 | 3.57 |
| LEFTY2 | 340657 | 1.00E−07 | 3.60 |
| APOL3 | 366289 | 1.00E−07 | 3.62 |
| ADRB2 | 127360 | 1.00E−07 | 3.65 |
| IMAGE:742574 | 742574 | 1.00E−07 | 3.69 |
| IMAGE:744926 | 744926 | 1.00E−07 | 3.73 |
| STK17A | 562904 | 1.00E−07 | 3.73 |
| LOC129531 | 487103 | 1.00E−07 | 3.75 |
| IMAGE:743722 | 743722 | 1.00E−07 | 3.76 |
| GATA4 | 781738 | 1.00E−07 | 3.78 |
| ECGF1 | 1632468 | 1.00E−07 | 3.86 |
| HLA-C | 727394 | 1.00E−07 | 3.95 |
| S100A11 | 143957 | 1.00E−07 | 3.98 |
| IMAGE:251047 | 251047 | 1.00E−07 | 3.98 |
| PPP3CB | 1116948 | 1.00E−07 | 4.00 |
| INDO | 2398980 | 1.00E−07 | 4.05 |
| PNOC | 34783 | 1.00E−07 | 4.05 |
| PRG1 | 1982430 | 1.00E−07 | 4.06 |
| CYP2C9 | 195712 | 1.00E−07 | 4.11 |
| TNFRSF25 | 1932202 | 1.00E−07 | 4.16 |
| BLR1 | 3058557 | 1.00E−07 | 4.20 |
| DCC | 2091389 | 1.00E−07 | 4.21 |
| IMAGE:5725785 | 5725785 | 1.00E−07 | 4.25 |
| BAI3 | 50491 | 1.00E−07 | 4.30 |
| WARS | 366359 | 1.00E−07 | 4.35 |
| C4orf6 | 743212 | 1.00E−07 | 4.41 |
| CCNT1 | 111460 | 1.00E−07 | 4.42 |
| ACY1 | 741988 | 1.00E−07 | 4.55 |
| FLJ12688 | 1735081 | 1.00E−07 | 4.66 |
| POLD3 | 825265 | 1.00E−07 | 4.72 |
| IRF4 | 682207 | 1.00E−07 | 4.75 |
| IMAGE:46991 | 46991 | 1.00E−07 | 4.80 |
| DKFZp667B0210 | 1844689 | 1.00E−07 | 4.83 |
| IMAGE:78294 | 78294 | 1.00E−07 | 4.85 |
| IL10RA | 258747 | 1.00E−07 | 4.92 |
| IMAGE:744468 | 744468 | 1.00E−07 | 4.95 |
| 38596 | 448163 | 1.00E−07 | 4.96 |
| PLEK | 724225 | 1.00E−07 | 4.98 |
| KRT1 | 1544175 | 1.00E−07 | 5.08 |
| DHCR24 | 742965 | 1.00E−07 | 5.12 |
| HLA-B | 964736 | 1.00E−07 | 5.20 |
| NIT2 | 742839 | 1.00E−07 | 5.21 |
| Leprot | 851264 | 1.00E−07 | 5.24 |
| LCK | 730410 | 1.00E−07 | 5.40 |
| IFI30 | 740931 | 1.00E−07 | 5.47 |
| SOCS3 | 152398 | 1.00E−07 | 5.54 |
| CERKL | 489326 | 1.00E−07 | 5.58 |
| IGHM | 276658 | 1.00E−07 | 5.62 |
| HLA-A | 1013595 | 1.00E−07 | 5.64 |
| IMAGE:1737165 | 1737165 | 1.00E−07 | 5.66 |
| TAP1 | 2158322 | 1.00E−07 | 5.75 |
| TIMP1 | 162246 | 1.00E−07 | 5.83 |
| MGC21518 | 738315 | 1.00E−07 | 5.89 |
| FAM12B | 744413 | 1.00E−07 | 5.91 |
| IMAGE:727496 | 727496 | 1.00E−07 | 6.19 |
| IMAGE:742555 | 742555 | 1.00E−07 | 6.22 |
| RARRES3 | 324225 | 1.00E−07 | 6.34 |
| IMAGE:134034 | 134034 | 1.00E−07 | 6.82 |
| IMAGE:731152 | 731152 | 1.00E−07 | 6.91 |
| DUSP5 | 33285 | 1.00E−07 | 7.14 |
| IMAGE:486779 | 486779 | 1.00E−07 | 7.15 |
| SPIB | 295093 | 1.00E−07 | 7.64 |
| IMAGE:178984 | 178984 | 1.00E−07 | 7.64 |
| HLA-DPB1 | 725548 | 1.00E−07 | 7.66 |
| C10orf128 | 485725 | 1.00E−07 | 7.74 |
| IMAGE:744366 | 744366 | 1.00E−07 | 7.78 |
| RARA | 159381 | 1.00E−07 | 7.81 |
| ALDH1A2 | 782730 | 1.00E−07 | 7.82 |
| CCL4 | 259552 | 1.00E−07 | 7.86 |
| NBL1 | 503874 | 1.00E−07 | 7.86 |
| TYROBP | 1146624 | 1.00E−07 | 7.91 |
| STX7 | 174396 | 1.00E−07 | 7.97 |
| LRRC17 | 120649 | 1.00E−07 | 8.59 |
| KLK13 | 743113 | 1.00E−07 | 9.07 |
| IMAGE:744911 | 744911 | 1.00E−07 | 9.25 |
| PDK2 | 114008 | 1.00E−07 | 9.38 |
| IMAGE:743237 | 743237 | 1.00E−07 | 9.51 |
| DKFZp586M1819 | 1072914 | 1.00E−07 | 9.79 |
| IMAGE:742899 | 742899 | 1.00E−07 | 9.94 |
| HLA-DRA | 153411 | 1.00E−07 | 10.40 |
| SLCO3A1 | 725696 | 1.00E−07 | 11.38 |
| PTPRA | 487130 | 1.00E−07 | 11.60 |
| IGKV1-5 | 155345 | 1.00E−07 | 11.62 |
| IGLC1 | 160163 | 1.00E−07 | 11.72 |
| SCGB2A1 | 2137932 | 1.00E−07 | 11.86 |
| FLJ27523 | 743246 | 1.00E−07 | 12.05 |
| LASP1 | 592598 | 1.00E−07 | 12.09 |
| HLA-DRB1 | 490710 | 1.00E−07 | 12.31 |
| IMAGE:745018 | 745018 | 1.00E−07 | 12.61 |
| RANBP2 | 245426 | 1.00E−07 | 12.66 |
| CD53 | 1324060 | 1.00E−07 | 12.90 |
| SALL4 | 726454 | 1.00E−07 | 12.91 |
| C9orf97 | 740885 | 1.00E−07 | 13.01 |
| CD37 | 1131054 | 1.00E−07 | 13.31 |
| HLA-DPA1 | 191881 | 1.00E−07 | 13.40 |
| NOS2A | 924826 | 1.00E−07 | 13.53 |
| PIK3CA | 2238108 | 1.00E−07 | 13.56 |
| PITX2 | 950695 | 1.00E−07 | 14.04 |
| MID1IP1 | 731762 | 1.00E−07 | 14.20 |
| LOC149478 | 714216 | 1.00E−07 | 14.84 |
| ZNF251 | 486401 | 1.00E−07 | 15.21 |
| CXCL9 | 1654256 | 1.00E−07 | 15.21 |
| PFC | 235934 | 1.00E−07 | 15.22 |
| IMAGE:1089025 | 1089025 | 1.00E−07 | 15.23 |
| UBE2N | 730271 | 1.00E−07 | 16.15 |
| UBD | 243741 | 1.00E−07 | 16.50 |
| ZNF451 | 742592 | 1.00E−07 | 16.61 |
| CD160 | 1580147 | 1.00E−07 | 17.77 |
| GTF2E1 | 668494 | 1.00E−07 | 19.41 |
| VPS13A | 745248 | 1.00E−07 | 19.68 |
| IMAGE:2361911 | 2361911 | 1.00E−07 | 19.85 |
| CFLAR | 427786 | 1.00E−07 | 20.20 |
| MGC40042 | 744755 | 1.00E−07 | 20.48 |
| PP35 | 726904 | 1.00E−07 | 21.69 |
| MTMR9 | 745223 | 1.00E−07 | 22.17 |
| IMAGE:738896 | 738896 | 1.00E−07 | 22.40 |
| TPD52 | 743259 | 1.00E−07 | 22.87 |
| HLA-DQA1 | 80109 | 1.00E−07 | 23.10 |
| PTGS1 | 1712870 | 1.00E−07 | 23.45 |
| OSBPL6 | 232002 | 1.00E−07 | 24.81 |
| IMAGE:742696 | 742696 | 1.00E−07 | 25.59 |
| IMAGE:342256 | 342256 | 1.00E−07 | 26.42 |
| IMAGE:744897 | 744897 | 1.00E−07 | 28.84 |
| IMAGE:726557 | 726557 | 1.00E−07 | 31.27 |
| RABGEF1 | 741389 | 1.00E−07 | 34.99 |
| MALT1 | 1420842 | 1.00E−07 | 41.74 |
| IMAGE:725758 | 725758 | 1.00E−07 | 42.90 |
| CCL19 | 1707527 | 1.00E−07 | 46.37 |
| IGH@ | 289337 | 1.00E−07 | 49.51 |
| IMAGE:739953 | 739953 | 1.00E−07 | 49.67 |
| APBA3 | 239086 | 1.00E−07 | 53.51 |
| IGKC | 713896 | 1.00E−07 | 59.97 |
| C21orf70 | 236386 | 1.00E−07 | 61.19 |
| IMAGE:1129745 | 1129745 | 1.00E−07 | 62.69 |
| IMAGE:2364998 | 2364998 | 1.00E−07 | 98.71 |
| IMAGE:504539 | 504539 | 1.00E−04 | 1.95 |
| STAT1 | 110101 | 1.00E−04 | 1.96 |
| CD81 | 1057773 | 1.00E−04 | 1.97 |
| BID | 128065 | 1.00E−04 | 2.21 |
| NEK6 | 725345 | 1.00E−04 | 2.26 |
| HDAC1 | 1896337 | 1.00E−04 | 2.29 |
| MCOLN1 | 726156 | 1.00E−04 | 2.85 |
| FLJ10439 | 731284 | 1.00E−04 | 2.88 |
| UBQLN1 | 292882 | 1.00E−04 | 2.99 |
| SLAMF8 | 288807 | 1.00E−04 | 3.05 |
| TRAF1 | 152844 | 1.00E−04 | 3.16 |
| SELL | 149910 | 1.00E−04 | 3.23 |
| MAP2K4 | 726147 | 1.00E−04 | 3.32 |
| PLA2G2A | 152802 | 1.00E−04 | 3.36 |
| LRRC14 | 725927 | 1.00E−04 | 3.37 |
| CXCR6 | 196115 | 1.00E−04 | 3.45 |
| CBL | 1578721 | 1.00E−04 | 3.45 |
| SEMA4D | 210587 | 1.00E−04 | 3.47 |
| DAXX | 742977 | 1.00E−04 | 3.60 |
| TCL1A | 200018 | 1.00E−04 | 3.68 |
| LOC203076 | 744756 | 1.00E−04 | 3.73 |
| MTX3 | 741661 | 1.00E−04 | 3.74 |
| G3BP | 667239 | 1.00E−04 | 3.78 |
| KLRK1 | 725473 | 1.00E−04 | 3.87 |
| CD58 | 2103105 | 1.00E−04 | 3.88 |
| AIF1 | 343867 | 1.00E−04 | 3.92 |
| RAP1A | 364271 | 1.00E−04 | 4.15 |
| RUNX3 | 122874 | 1.00E−04 | 4.20 |
| SYT11 | 180950 | 1.00E−04 | 4.30 |
| RGS3 | 232507 | 1.00E−04 | 4.35 |
| SFRP4 | 285693 | 1.00E−04 | 4.36 |
| HPS6 | 723938 | 1.00E−04 | 4.39 |
| CARD11 | 665651 | 1.00E−04 | 4.72 |
| APOL1 | 665632 | 1.00E−04 | 4.79 |
| NCK1 | 1326169 | 1.00E−04 | 4.98 |
| IL16 | 204381 | 1.00E−04 | 5.00 |
| CKIP-1 | 727207 | 1.00E−04 | 5.03 |
| HCLS1 | 665452 | 1.00E−04 | 5.05 |
| CD74 | 117072 | 1.00E−04 | 5.14 |
| TYR | 271985 | 1.00E−04 | 5.15 |
| SPDEF | 1188588 | 1.00E−04 | 5.50 |
| BATF | 1929371 | 1.00E−04 | 6.03 |
| ARHGAP25 | 665982 | 1.00E−04 | 6.13 |
| TXNDC5 | 471210 | 1.00E−04 | 6.38 |
| PSCDBP | 824531 | 1.00E−04 | 7.09 |
| CD38 | 1352408 | 1.00E−04 | 7.22 |
| NOD27 | 667435 | 1.00E−04 | 7.22 |
| SARS | 248811 | 1.00E−04 | 7.34 |
| C18orf51 | 742649 | 1.00E−04 | 7.47 |
| TJP3 | 741919 | 1.00E−04 | 7.75 |
| IMAGE:742853 | 742853 | 1.00E−04 | 7.90 |
| POU2AF1 | 1541958 | 1.00E−04 | 8.06 |
| CD79A | 1056782 | 1.00E−04 | 8.58 |
| EPPB9 | 668454 | 1.00E−04 | 9.03 |
| LYZ | 1256485 | 1.00E−04 | 9.16 |
| CXCR4 | 49725 | 1.00E−04 | 9.19 |
| CBFA2T2 | 43629 | 1.00E−04 | 10.03 |
| IMAGE:730865 | 730865 | 1.00E−04 | 13.00 |
| CD48 | 667144 | 1.00E−04 | 13.03 |
| FLJ39378 | 724042 | 1.00E−04 | 13.51 |
| NEDD9 | 667602 | 1.00E−04 | 17.38 |
| MGC26647 | 745326 | 1.00E−04 | 20.55 |
| ZBTB33 | 725622 | 1.00E−04 | 21.42 |
| TAPBP | 723753 | 2.00E−04 | 2.12 |
| ISGF3G | 724588 | 2.00E−04 | 2.25 |
| IMAGE:744881 | 744881 | 2.00E−04 | 2.35 |
| GFPT2 | 485085 | 2.00E−04 | 2.36 |
| IMAGE:745125 | 745125 | 2.00E−04 | 2.44 |
| CASP3 | 823680 | 2.00E−04 | 2.72 |
| LOC283658 | 666755 | 2.00E−04 | 9.05 |
| CD79B | 155717 | 2.00E−04 | 10.51 |
| IMAGE:727154 | 727154 | 3.00E−04 | 2.38 |
| APOE | 1870594 | 3.00E−04 | 3.26 |
| PREB | 740347 | 3.00E−04 | 4.99 |
| IMAGE:729489 | 729489 | 3.00E−04 | 5.80 |
| CXCL14 | 345034 | 3.00E−04 | 9.35 |
| FLJ12592 | 252489 | 4.00E−04 | 1.96 |
| LOC440712 | 725268 | 4.00E−04 | 2.14 |
| CSF1 | 124554 | 4.00E−04 | 2.40 |
| ESRRG | 44064 | 5.00E−04 | 9.39 |
| NOMO1 | 134615 | 7.00E−04 | 1.77 |
| C19orf33 | 379540 | 7.00E−04 | 2.37 |
| MGP | 590264 | 7.00E−04 | 3.05 |
| CXCL6 | 2108870 | 7.00E−04 | 4.29 |
| STAT2 | 2306096 | 8.00E−04 | 1.81 |
| MLL | 80688 | 8.00E−04 | 1.88 |
| HMGB2 | 884365 | 8.00E−04 | 1.92 |
| CCL11 | 502658 | 8.00E−04 | 2.04 |
| SPI1 | 1285305 | 9.00E−04 | 1.84 |
| NOTCH2 | 1641901 | 1.10E−03 | 2.97 |
| CD5 | 356841 | 1.50E−03 | 1.82 |
| SLC9A3R1 | 773286 | 1.50E−03 | 2.15 |
| S100A4 | 868577 | 1.60E−03 | 3.88 |
| SLC6A1 | 177967 | 2.00E−03 | 1.66 |
| FLJ35794 | 364204 | 2.30E−03 | 2.21 |
| FXYD1 | 204686 | 2.40E−03 | 1.72 |
| UBE1L | 250883 | 3.10E−03 | 1.88 |
| F10 | 310519 | 3.30E−03 | 1.68 |
| NCB5OR | 743367 | 3.90E−03 | 2.21 |
| IMAGE:740105 | 740105 | 4.30E−03 | 1.70 |
| CFH | 665784 | 6.80E−03 | 2.37 |
| C4A | 724366 | 7.70E−03 | 2.75 |
| MRC2 | 235882 | 7.90E−03 | 1.59 |
| MYC | 129438 | 7.90E−03 | 1.96 |
| NRIP1 | 38775 | 8.20E−03 | 1.64 |
| NUP214 | 382612 | 8.60E−03 | 1.86 |
| DCN | 666410 | 1.84E−02 | 1.85 |
| COL8A2 | 486204 | 2.00E−02 | 1.64 |
| TLN1 | 187482 | 2.08E−02 | 1.69 |
| ELK4 | 236155 | 2.17E−02 | 1.50 |
| FLJ22794 | 137454 | 2.43E−02 | 1.54 |
| IL11RA | 1101773 | 2.59E−02 | 1.63 |
| KIAA1576 | 743426 | 2.91E−02 | 1.51 |
| APOD | 838611 | 2.98E−02 | 2.89 |
| GABRE | 209137 | 3.67E−02 | 1.45 |
| NRP1 | 666061 | 3.95E−02 | 1.44 |
| ABCA3 | 52740 | 4.99E−01 | 1.13 |
| AAAS | 740617 | 3.00E−07 | 3.34 |
| CXCL6 | 2108870 | 1.00E−07 | 4.29 |
| CARD11 | 665651 | 1.00E−07 | 4.72 |
| DKFZp667B0210 | 1844689 | 1.00E−07 | 4.83 |
| CD38 | 1352408 | 1.00E−07 | 7.22 |
| CD79A | 1056782 | 1.00E−07 | 8.58 |
| EPPB9 | 668454 | 1.00E−07 | 9.03 |
| CBFA2T2 | 43629 | 1.00E−07 | 10.03 |
| CD79B | 155717 | 1.00E−07 | 10.51 |

TABLE 27

| | GD | Under | |
|---|---|---|---|
| name | cloid | p-value | fold |
| DCXR | 724596 | 3.00E−03 | 0.61 |
| VEGFC | 503189 | 1.65E−02 | 0.65 |
| LARS | 135407 | 3.74E−02 | 0.71 |
| AURKC | 731021 | 4.05E−02 | 0.62 |
| MRPL45 | 364717 | 4.33E−02 | 0.64 |
| IMAGE:23817 | 23817 | 3.65E−04 | 0.52 |
| DHX57 | 24623 | 1.69E−03 | 0.59 |
| DCXR | 724596 | 3.39E−03 | 0.61 |
| IMAGE:180851 | 180851 | 5.36E−03 | 0.61 |

TABLE 28

| | GD | Over | |
|---|---|---|---|
| name | cloid | p-value | fold |
| ATP6V1G2 | 726424 | 2.10E−03 | 2.22 |
| KIAA1737 | 122063 | 3.40E−03 | 1.67 |
| IMAGE:729489 | 729489 | 5.90E−03 | 2.95 |
| ISGF3G | 724588 | 6.30E−03 | 1.72 |
| AIF1 | 343867 | 6.80E−03 | 2.21 |
| PPP3CB | 1116948 | 7.70E−03 | 1.82 |
| IMAGE:727154 | 727154 | 7.70E−03 | 1.81 |
| ECGF1 | 1632468 | 8.00E−03 | 1.99 |
| CD81 | 1057773 | 8.70E−03 | 1.61 |
| CD74 | 117072 | 8.80E−03 | 2.48 |
| TNFRSF1B | 73703 | 9.10E−03 | 1.58 |
| GPR124 | 486493 | 1.01E−02 | 1.52 |
| MGP | 590264 | 1.07E−02 | 2.38 |
| MAP2K4 | 726147 | 1.22E−02 | 1.69 |
| S100A8 | 1089513 | 1.23E−02 | 1.86 |
| PLEK | 724225 | 1.55E−02 | 1.89 |
| HLA-DQA1 | 80109 | 1.59E−02 | 3.06 |
| IMAGE:744385 | 744385 | 1.63E−02 | 1.70 |
| CXCL9 | 1654256 | 1.81E−02 | 2.95 |
| HLA-B | 964736 | 1.83E−02 | 1.95 |
| NOD27 | 667435 | 1.89E−02 | 1.94 |
| HNRPA3 | 365349 | 1.97E−02 | 1.48 |
| IMAGE:742696 | 742696 | 2.02E−02 | 3.53 |
| SPIB | 295093 | 2.08E−02 | 2.28 |
| CD3G | 110938 | 2.10E−02 | 1.55 |
| CYP2C9 | 195712 | 2.11E−02 | 1.80 |
| RANBP2 | 245426 | 2.24E−02 | 2.49 |
| ADRB2 | 127360 | 2.28E−02 | 1.54 |
| RUNX3 | 122874 | 2.29E−02 | 1.62 |
| 38596 | 448163 | 2.29E−02 | 1.82 |
| CERKL | 489326 | 2.35E−02 | 1.75 |
| TIMP3 | 501476 | 2.40E−02 | 1.86 |
| LILRB2 | 202897 | 2.43E−02 | 1.59 |
| C13orf12 | 23121 | 2.47E−02 | 1.59 |
| IMAGE:744911 | 744911 | 2.57E−02 | 2.06 |
| C4orf6 | 743212 | 2.72E−02 | 1.68 |
| CXCR4 | 49725 | 2.72E−02 | 2.01 |
| HLA-DPB1 | 725548 | 2.77E−02 | 2.06 |
| HLA-DPA1 | 191881 | 2.86E−02 | 2.93 |
| IMAGE:744926 | 744926 | 2.88E−02 | 1.60 |
| HCLS1 | 665452 | 2.89E−02 | 1.67 |
| TAP1 | 2158322 | 3.11E−02 | 1.76 |
| HLA-C | 727394 | 3.14E−02 | 1.59 |
| BST2 | 811024 | 3.21E−02 | 1.48 |
| CCL4 | 259552 | 3.25E−02 | 2.14 |
| RABGEF1 | 741389 | 3.34E−02 | 4.28 |
| RGS3 | 232507 | 3.38E−02 | 1.50 |
| B2M | 342416 | 3.65E−02 | 1.68 |
| PIK3CA | 2238108 | 3.66E−02 | 2.19 |
| RIPK3 | 667313 | 3.66E−02 | 1.46 |
| RARRES3 | 324225 | 3.73E−02 | 1.99 |
| WARS | 366359 | 3.84E−02 | 1.89 |
| SPDEF | 1188588 | 3.90E−02 | 1.61 |
| IMAGE:742899 | 742899 | 3.90E−02 | 1.86 |
| CASP3 | 823680 | 3.95E−02 | 1.65 |
| ALDH1A2 | 782730 | 3.95E−02 | 1.76 |
| IL16 | 204381 | 4.00E−02 | 1.58 |
| CKIP-1 | 727207 | 4.02E−02 | 1.66 |
| UBD | 243741 | 4.11E−02 | 2.22 |
| LRRC17 | 120649 | 4.23E−02 | 1.78 |
| PLAC9 | 366115 | 4.26E−02 | 1.69 |
| C9orf97 | 740885 | 4.30E−02 | 2.15 |
| CDH16 | 726763 | 4.35E−02 | 1.83 |
| IL10RA | 258747 | 4.35E−02 | 1.56 |
| IMAGE:727554 | 727554 | 4.39E−02 | 1.45 |
| HLA-DRA | 153411 | 4.74E−02 | 2.66 |
| LAIR1 | 665566 | 4.75E−02 | 1.42 |
| TRAF5 | 1286238 | 4.81E−02 | 1.42 |
| TERF1 | 135773 | 2.81E−02 | 1.45 |
| IMAGE:136686 | 136686 | 6.29E−02 | 1.48 |
| RAI14 | 731711 | 8.19E−03 | 1.56 |
| MLH3 | 250771 | 8.59E−03 | 1.59 |
| KIAA1737 | 122063 | 1.78E−03 | 1.67 |
| IMAGE:744385 | 744385 | 1.37E−02 | 1.70 |
| D21S2056E | 740140 | 2.44E−03 | 1.72 |
| SSX1 | 262894 | 5.97E−04 | 1.76 |
| IMAGE:53081 | 53081 | 3.35E−03 | 1.77 |
| LOC150837 | 258698 | 5.84E−04 | 1.81 |
| MGC5566 | 53119 | 1.74E−03 | 1.85 |
| TTC19 | 136366 | 1.57E−04 | 1.87 |
| BDKRB2 | 665674 | 6.16E−03 | 2.15 |
| IMAGE:178161 | 178161 | 6.99E−04 | 2.53 |
| IMAGE:729489 | 729489 | 1.28E−03 | 2.95 |

TABLE 29

| | FTA-a | Under | |
|---|---|---|---|
| name | cloid | p-value | fold |
| VDAC1 | 486221 | 1.00E−07 | 0.67 |
| PLCG1 | 1174287 | 1.00E−04 | 0.67 |
| NRP1 | 666061 | 2.00E−04 | 0.72 |
| IMAGE:504539 | 504539 | 2.00E−04 | 0.75 |
| IMAGE:742837 | 742837 | 2.00E−04 | 0.73 |
| C1QBP | 173371 | 4.00E−04 | 0.73 |
| ALDH1A3 | 486189 | 5.00E−04 | 0.69 |
| IMAGE:682585 | 682585 | 6.00E−04 | 0.76 |
| RPS6KA5 | 258966 | 8.00E−04 | 0.77 |
| NR2F1 | 253386 | 9.00E−04 | 0.74 |
| IMAGE:213529 | 213529 | 1.00E−03 | 0.75 |
| COX7B | 566862 | 1.10E−03 | 0.70 |
| GFPT2 | 485085 | 1.20E−03 | 0.73 |
| IDH3B | 723755 | 1.20E−03 | 0.77 |
| IMAGE:382423 | 382423 | 1.30E−03 | 0.70 |
| C6orf216 | 731742 | 1.70E−03 | 0.74 |
| SPI1 | 1285305 | 1.80E−03 | 0.77 |
| MRC2 | 235882 | 1.80E−03 | 0.77 |
| LOC56251 | 137478 | 2.00E−03 | 0.78 |
| SLC36A3 | 731115 | 2.20E−03 | 0.80 |
| IMAGE:727289 | 727289 | 2.70E−03 | 0.67 |
| LEMD1 | 731047 | 2.90E−03 | 0.71 |
| ELK4 | 236155 | 3.70E−03 | 0.79 |
| RBBP4 | 705147 | 3.90E−03 | 0.80 |
| CLGN | 1049033 | 4.00E−03 | 0.76 |
| KLF4 | 188232 | 4.00E−03 | 0.68 |
| IMAGE:666794 | 666794 | 4.30E−03 | 0.75 |
| SIAT8C | 382069 | 4.30E−03 | 0.78 |
| S100A10 | 119939 | 4.40E−03 | 0.70 |
| FCHO1 | 742763 | 4.50E−03 | 0.79 |
| NAT5 | 731073 | 4.70E−03 | 0.78 |
| ABCA8 | 284828 | 5.20E−03 | 0.75 |
| IMAGE:743603 | 743603 | 5.60E−03 | 0.79 |
| IMAGE:665649 | 665649 | 6.80E−03 | 0.79 |
| IMAGE:745072 | 745072 | 6.90E−03 | 0.72 |
| STK17A | 562904 | 8.70E−03 | 0.78 |
| ARVCF | 40633 | 8.90E−03 | 0.81 |
| KLRK1 | 725473 | 8.90E−03 | 0.79 |
| IMAGE:744616 | 744616 | 9.40E−03 | 0.80 |
| CD5 | 356841 | 1.00E−02 | 0.81 |
| LEFTY2 | 340657 | 1.09E−02 | 0.77 |
| MYC | 129438 | 1.10E−02 | 0.75 |
| CHRDL2 | 485872 | 1.20E−02 | 0.81 |
| HSPC159 | 365045 | 1.29E−02 | 0.80 |
| HRK | 767779 | 1.44E−02 | 0.72 |
| LRPAP1 | 587186 | 1.45E−02 | 0.79 |
| LARS | 135407 | 1.47E−02 | 0.83 |
| PDK1 | 1645668 | 1.48E−02 | 0.83 |
| IMAGE:744899 | 744899 | 1.48E−02 | 0.82 |
| IMAGE:382521 | 382521 | 1.54E−02 | 0.77 |
| ANP32E | 382738 | 1.55E−02 | 0.75 |
| RAP1A | 364271 | 1.65E−02 | 0.79 |
| SIAT8F | 667110 | 1.72E−02 | 0.76 |
| IGSF10 | 682276 | 1.79E−02 | 0.82 |
| MFAP3L | 726821 | 1.89E−02 | 0.84 |
| DOK5 | 25664 | 1.97E−02 | 0.77 |
| IMAGE:21496 | 21496 | 1.98E−02 | 0.79 |
| IMAGE:729875 | 729875 | 2.06E−02 | 0.80 |
| DEFB1 | 665086 | 2.12E−02 | 0.72 |
| RPL27A | 178255 | 2.13E−02 | 0.70 |
| CKS2 | 725454 | 2.22E−02 | 0.76 |
| LILRB5 | 71428 | 2.22E−02 | 0.79 |
| MAK | 382002 | 2.24E−02 | 0.79 |
| KIAA1754L | 365056 | 2.28E−02 | 0.79 |
| DEK | 1016390 | 2.50E−02 | 0.83 |
| IMAGE:731616 | 731616 | 2.57E−02 | 0.71 |
| CBL | 1578721 | 2.72E−02 | 0.79 |
| ME3 | 724238 | 2.74E−02 | 0.76 |
| BAP1 | 1012990 | 3.01E−02 | 0.79 |
| CD58 | 2103105 | 3.13E−02 | 0.82 |
| BIN1 | 2384812 | 3.43E−02 | 0.84 |
| CLECSF12 | 258865 | 3.49E−02 | 0.65 |
| CASP5 | 341763 | 3.51E−02 | 0.73 |
| MRPL45 | 364717 | 3.59E−02 | 0.81 |
| PPP3CB | 1116948 | 3.62E−02 | 0.81 |
| NCK1 | 1326169 | 3.76E−02 | 0.80 |
| GPI | 741474 | 3.87E−02 | 0.83 |
| SAFB | 42280 | 4.11E−02 | 0.79 |
| GNAI1 | 753215 | 4.20E−02 | 0.82 |
| PRG1 | 1982430 | 4.37E−02 | 0.77 |
| HMG20A | 731277 | 4.41E−02 | 0.78 |
| PCSK6 | 31924 | 4.56E−02 | 0.80 |
| CYP2C9 | 195712 | 4.68E−02 | 0.79 |
| LOC285961 | 742542 | 4.79E−02 | 0.86 |
| HCLS1 | 665452 | 4.91E−02 | 0.80 |
| PLCG1 | 1174287 | 8.36E−05 | 0.67 |
| PDK4 | 487379 | 1.00E−02 | 0.70 |
| IMAGE:24065 | 24065 | 4.52E−04 | 0.75 |

TABLE 30

| | FTA-a | Over | |
|---|---|---|---|
| name | cloid | p-value | fold |
| TBC1D17 | 739955 | 1.00E−07 | 1.56 |
| PLXNB2 | 1420676 | 1.00E−07 | 1.70 |
| ZNF76 | 745003 | 1.00E−07 | 1.51 |
| KIAA1196 | 738938 | 1.00E−07 | 1.52 |
| BCL2L11 | 300194 | 1.00E−07 | 1.42 |
| ASGR1 | 25883 | 1.00E−07 | 1.75 |
| RAD51L1 | 295412 | 1.00E−07 | 1.66 |
| PRUNE | 364324 | 1.00E−07 | 1.41 |
| PH-4 | 730938 | 1.00E−07 | 1.34 |
| IMAGE:744055 | 744055 | 1.00E−07 | 1.73 |
| DAP | 725371 | 1.00E−04 | 1.51 |
| CABIN1 | 1844968 | 1.00E−04 | 1.59 |
| RPS6KA2 | 22711 | 1.00E−04 | 1.57 |
| UBE1L | 250883 | 1.00E−04 | 1.41 |
| SLC25A23 | 741954 | 2.00E−04 | 1.47 |
| PLA2G6 | 744087 | 3.00E−04 | 1.43 |
| TLN1 | 187482 | 4.00E−04 | 1.44 |
| IMAGE:744505 | 744505 | 4.00E−04 | 1.34 |
| IL11RA | 1101773 | 7.00E−04 | 1.40 |
| PVRL2 | 725364 | 8.00E−04 | 1.30 |
| LGALS3BP | 742100 | 8.00E−04 | 1.56 |
| LOC146909 | 726353 | 9.00E−04 | 1.29 |
| CaMKIINalpha | 173820 | 1.00E−03 | 1.35 |
| DNALI1 | 782688 | 1.20E−03 | 1.45 |
| C4A | 724366 | 1.30E−03 | 1.76 |
| IMAGE:731726 | 731726 | 1.70E−03 | 1.44 |
| FCGBP | 154172 | 1.70E−03 | 1.71 |
| KCNC3 | 53333 | 1.90E−03 | 1.36 |
| IMAGE:1716286 | 1716286 | 1.90E−03 | 1.52 |
| ANKRD20B | 745106 | 2.50E−03 | 1.40 |
| RIPK3 | 667313 | 2.50E−03 | 1.29 |
| KLF1 | 208991 | 2.60E−03 | 1.49 |
| IMAGE:744385 | 744385 | 3.50E−03 | 1.35 |
| E2F5 | 809828 | 3.90E−03 | 1.52 |
| PDLIM1 | 135689 | 4.30E−03 | 1.38 |
| SH3RF2 | 744797 | 4.40E−03 | 1.35 |
| SEMA7A | 135941 | 4.60E−03 | 1.70 |
| PBXIP1 | 366042 | 5.40E−03 | 1.33 |
| EPHB6 | 172982 | 8.00E−03 | 1.44 |
| HSPB1 | 23827 | 9.50E−03 | 1.30 |
| APLP2 | 549054 | 1.19E−02 | 1.33 |
| CDH1 | 214008 | 1.22E−02 | 1.30 |
| USP13 | 666007 | 1.23E−02 | 1.27 |
| IMAGE:136801 | 136801 | 1.27E−02 | 1.21 |
| IMAGE:724416 | 724416 | 1.36E−02 | 1.36 |
| PAX8 | 545475 | 1.53E−02 | 1.36 |
| DCI | 667892 | 1.55E−02 | 1.22 |
| SNRP70 | 729971 | 1.60E−02 | 1.31 |
| PEMT | 742580 | 1.78E−02 | 1.20 |
| C10orf116 | 740941 | 1.83E−02 | 1.42 |
| DKFZp434M202 | 743118 | 2.07E−02 | 1.21 |
| KIAA0789 | 33621 | 2.45E−02 | 1.26 |
| NDUFA7 | 364469 | 2.60E−02 | 1.19 |
| LOC340073 | 731404 | 2.76E−02 | 1.24 |
| ITPR1 | 667348 | 2.86E−02 | 1.22 |
| MT1F | 78353 | 3.11E−02 | 1.45 |
| FOXO1A | 151247 | 3.31E−02 | 1.20 |
| NIFIE14 | 472160 | 3.34E−02 | 1.19 |
| ID1 | 1087348 | 3.95E−02 | 1.34 |
| HSPB3 | 502598 | 3.96E−02 | 1.35 |
| ZFOC1 | 744606 | 4.07E−02 | 1.20 |
| ALCAM | 172828 | 4.14E−02 | 1.17 |
| POLR2A | 740130 | 4.49E−02 | 1.19 |
| LOC400451 | 667174 | 4.89E−02 | 1.22 |
| IMAGE:745465 | 745465 | 2.58E−02 | 1.24 |
| CaMKIINalpha | 173820 | 9.52E−04 | 1.35 |
| BAT3 | 24392 | 8.11E−04 | 1.42 |
| BCL2L11 | 300194 | 6.00E−06 | 1.42 |
| KLF1 | 208991 | 3.01E−03 | 1.49 |
| FLJ14981 | 24532 | 1.84E−04 | 1.50 |
| DAP | 725371 | 6.20E−06 | 1.51 |
| RPS6KA2 | 22711 | 3.77E−05 | 1.57 |
| TSPAN1 | 376003 | 3.52E−05 | 1.57 |
| CABIN1 | 1844968 | 1.12E−05 | 1.59 |
| COL18A1 | 359202 | 3.20E−05 | 1.60 |
| RAD51L1 | 295412 | 4.23E−05 | 1.66 |
| ASGR1 | 25883 | 1.25E−05 | 1.75 |

TABLE 31

| | FTA- | | |
|---|---|---|---|
| | atypical | Under | |
| name | cloid | p-value | fold |
| NBL1 | 503874 | 2.47E−04 | 0.35 |
| SCNN1A | 741305 | 2.37E−05 | 0.41 |
| KLF1 | 208991 | 3.72E−05 | 0.43 |
| NCB5OR | 743367 | 2.03E−03 | 0.56 |
| EIF1AY | 380394 | 4.02E−05 | 0.57 |
| IFNAR2 | 123950 | 2.56E−04 | 0.61 |
| ZNF415 | 23939 | 4.94E−03 | 0.63 |
| WDR1 | 714196 | 2.06E−04 | 0.65 |
| KLK2 | 1102600 | 1.39E−03 | 0.66 |
| KLF1 | 208991 | 1.00E−07 | 0.43 |
| CREB3L2 | 136399 | 2.00E−04 | 0.48 |
| NBL1 | 503874 | 3.00E−04 | 0.35 |
| IFNAR2 | 123950 | 4.00E−04 | 0.61 |
| KLK2 | 1102600 | 4.00E−04 | 0.66 |
| CD3D | 377560 | 1.10E−03 | 0.56 |
| NCB5OR | 743367 | 2.40E−03 | 0.56 |
| NTRK2 | 2048801 | 2.50E−03 | 0.53 |
| SAFB | 42280 | 3.10E−03 | 0.58 |
| TOSO | 813174 | 3.20E−03 | 0.57 |
| LRP1 | 30219 | 3.30E−03 | 0.63 |
| CBL | 1578721 | 4.00E−03 | 0.61 |
| SIAT8B | 33133 | 4.10E−03 | 0.71 |
| COL27A1 | 379517 | 4.30E−03 | 0.69 |
| FXYD1 | 204686 | 4.30E−03 | 0.69 |
| SIGLEC7 | 743331 | 4.90E−03 | 0.70 |
| FLJ11184 | 384404 | 5.00E−03 | 0.58 |
| MGC23909 | 731598 | 5.00E−03 | 0.54 |
| NEK6 | 725345 | 5.30E−03 | 0.69 |
| APOD | 838611 | 6.10E−03 | 0.38 |
| NME7 | 743982 | 6.30E−03 | 0.49 |
| C19orf33 | 379540 | 6.50E−03 | 0.63 |
| IMAGE:743619 | 743619 | 6.90E−03 | 0.70 |
| CCDC6 | 487848 | 7.70E−03 | 0.68 |
| REV1L | 738916 | 8.70E−03 | 0.52 |
| IMAGE:744074 | 744074 | 1.00E−02 | 0.51 |
| BAI3 | 50491 | 1.05E−02 | 0.65 |
| IMAGE:1089025 | 1089025 | 1.10E−02 | 0.46 |
| DF | 666128 | 1.11E−02 | 0.49 |
| DUSP24 | 740158 | 1.12E−02 | 0.48 |
| CASP5 | 341763 | 1.33E−02 | 0.57 |
| C10orf116 | 740941 | 1.39E−02 | 0.58 |
| 37500 | 729542 | 1.40E−02 | 0.63 |
| IMAGE:251047 | 251047 | 1.40E−02 | 0.66 |
| DHCR24 | 742965 | 1.41E−02 | 0.57 |
| GSS | 140405 | 1.42E−02 | 0.68 |
| CCNT1 | 111460 | 1.44E−02 | 0.67 |
| PARD3 | 724642 | 1.46E−02 | 0.75 |
| SPON2 | 723923 | 1.48E−02 | 0.72 |
| LOC149478 | 714216 | 1.51E−02 | 0.48 |
| IMAGE:731616 | 731616 | 1.51E−02 | 0.57 |
| CAV1 | 309645 | 1.51E−02 | 0.63 |
| C20orf19 | 366032 | 1.56E−02 | 0.74 |
| IMAGE:740105 | 740105 | 1.66E−02 | 0.73 |
| IMAGE:744926 | 744926 | 1.71E−02 | 0.69 |
| APIN | 364706 | 1.71E−02 | 0.69 |
| MGC99813 | 739097 | 1.75E−02 | 0.46 |
| FLJ12688 | 1735081 | 1.78E−02 | 0.65 |
| SLC17A2 | 207920 | 1.85E−02 | 0.53 |
| AMICA1 | 487436 | 1.93E−02 | 0.68 |
| CRABP1 | 739193 | 1.95E−02 | 0.39 |
| IMAGE:177857 | 177857 | 1.98E−02 | 0.68 |
| S100A8 | 1089513 | 2.00E−02 | 0.66 |
| IMAGE:743603 | 743603 | 2.01E−02 | 0.74 |
| DCC | 2091389 | 2.16E−02 | 0.72 |
| CFH | 665784 | 2.19E−02 | 0.59 |
| RNF32 | 731422 | 2.35E−02 | 0.62 |
| LILRB5 | 71428 | 2.37E−02 | 0.70 |
| KCNQ2 | 179534 | 2.38E−02 | 0.69 |
| BAP1 | 1012990 | 2.40E−02 | 0.68 |
| IMAGE:666315 | 666315 | 2.41E−02 | 0.70 |
| TNFRSF25 | 1932202 | 2.46E−02 | 0.66 |
| AQP11 | 713831 | 2.51E−02 | 0.65 |
| MEOX1 | 760065 | 2.59E−02 | 0.68 |
| ARHGAP10 | 731380 | 2.60E−02 | 0.66 |
| APOL1 | 665632 | 2.65E−02 | 0.54 |
| MUTED | 731202 | 2.66E−02 | 0.67 |
| ISG20 | 740604 | 2.71E−02 | 0.70 |
| DEFB1 | 665086 | 2.86E−02 | 0.61 |
| IMAGE:136686 | 136686 | 2.95E−02 | 0.72 |
| CDH16 | 726763 | 3.12E−02 | 0.63 |
| BIC | 743270 | 3.21E−02 | 0.74 |
| MMRN1 | 759865 | 3.24E−02 | 0.74 |
| IMAGE:364741 | 364741 | 3.29E−02 | 0.67 |
| SIAT7B | 823590 | 3.62E−02 | 0.74 |
| BAZ2B | 609631 | 3.64E−02 | 0.78 |
| OBSCN | 730926 | 3.79E−02 | 0.79 |
| PLCG2 | 201467 | 3.83E−02 | 0.72 |
| F10 | 310519 | 3.85E−02 | 0.77 |
| ADRB2 | 127360 | 3.85E−02 | 0.76 |
| RODH | 471641 | 3.90E−02 | 0.51 |
| DAXX | 742977 | 4.00E−02 | 0.77 |
| VAV1 | 80384 | 4.07E−02 | 0.60 |
| ME3 | 724238 | 4.21E−02 | 0.67 |
| SLPI | 378813 | 4.27E−02 | 0.67 |
| JUP | 35628 | 4.42E−02 | 0.64 |
| LOC129531 | 487103 | 4.66E−02 | 0.71 |
| MATN2 | 28584 | 4.74E−02 | 0.67 |
| TFF1 | 1075949 | 4.74E−02 | 0.65 |
| MCC | 731305 | 4.75E−02 | 0.63 |

TABLE 32

| | FTA- | | |
|---|---|---|---|
| | atypical | Over | |
| name | cloid | p-value | fold |
| ICOSLG | 2074228 | 1.12E−03 | 1.55 |
| ETF1 | 146976 | 6.36E−04 | 1.63 |
| ANK2 | 2139315 | 1.31E−03 | 1.68 |
| FLJ10748 | 726830 | 4.24E−05 | 1.71 |
| SNCB | 50202 | 4.68E−05 | 1.99 |
| IGFBP3 | 269873 | 9.68E−05 | 2.06 |
| TSC | 745490 | 1.95E−04 | 2.61 |
| CITED1 | 265558 | 1.21E−05 | 3.53 |
| IMAGE:687667 | 687667 | 1.00E−07 | 3.90 |
| CITED1 | 265558 | 1.00E−07 | 3.53 |
| DJ462O23.2 | 738970 | 1.00E−07 | 1.63 |
| FLJ10748 | 726830 | 1.00E−04 | 1.71 |
| ETF1 | 146976 | 3.00E−04 | 1.63 |
| TSC | 745490 | 4.00E−04 | 2.61 |
| CA11 | 282587 | 5.00E−04 | 1.53 |
| PSAT1 | 366388 | 9.00E−04 | 1.50 |
| ICOSLG | 2074228 | 1.50E−03 | 1.55 |
| IMAGE:669188 | 669188 | 1.80E−03 | 1.71 |
| IMAGE:668536 | 668536 | 2.50E−03 | 1.97 |
| PBXIP1 | 366042 | 3.00E−03 | 1.60 |
| ABCC3 | 208097 | 3.10E−03 | 2.12 |
| IMP-2 | 743774 | 3.30E−03 | 2.13 |
| TM4SF8 | 713647 | 3.60E−03 | 1.45 |
| KCNK5 | 134978 | 3.90E−03 | 1.61 |
| IMAGE:731751 | 731751 | 4.80E−03 | 1.80 |
| BCL2L11 | 300194 | 6.80E−03 | 1.40 |
| MESDC2 | 713653 | 8.90E−03 | 1.39 |
| CHRAC1 | 724276 | 9.20E−03 | 1.36 |
| ECM1 | 301122 | 1.10E−02 | 2.21 |
| SPATA12 | 730300 | 1.37E−02 | 1.37 |
| FRMD4B | 669564 | 1.58E−02 | 1.51 |
| SCEL | 668239 | 1.58E−02 | 1.38 |
| IMAGE:738332 | 738332 | 1.74E−02 | 1.82 |
| MGC17299 | 713422 | 1.95E−02 | 1.32 |
| CLDN1 | 594279 | 2.21E−02 | 2.31 |
| S100A13 | 1104732 | 2.30E−02 | 1.39 |
| PDCD4 | 665376 | 2.43E−02 | 1.44 |
| PH-4 | 730938 | 2.47E−02 | 1.30 |
| LOC400451 | 667174 | 2.90E−02 | 1.40 |
| IMAGE:1877668 | 1877668 | 2.99E−02 | 1.33 |
| CLDN7 | 300268 | 3.01E−02 | 1.29 |
| LRPAP1 | 587186 | 3.20E−02 | 1.39 |
| QPCT | 711918 | 3.46E−02 | 1.60 |
| NR2F2 | 72744 | 3.48E−02 | 1.34 |
| IL19 | 1932168 | 3.80E−02 | 1.42 |
| CKLFSF4 | 143759 | 3.82E−02 | 1.28 |
| CICE | 725791 | 3.82E−02 | 1.30 |
| ACE2 | 730971 | 3.99E−02 | 1.34 |
| MGC19604 | 687468 | 4.18E−02 | 1.29 |
| CD63 | 125552 | 4.69E−02 | 1.26 |
| FLT4 | 668815 | 4.70E−02 | 1.28 |
| NFKB1 | 687782 | 4.99E−02 | 1.32 |
| CKS2 | 725454 | 5.03E−02 | 1.43 |

TABLE 33

| | FTA-b | Under | |
|---|---|---|---|
| name | cloid | p-value | fold |
| IFI30 | 740931 | 1.00E−07 | 0.41 |
| ISG20 | 740604 | 1.00E−07 | 0.60 |
| ALCAM | 172828 | 1.00E−04 | 0.68 |
| LOC440712 | 725268 | 5.00E−04 | 0.67 |
| IMAGE:136014 | 136014 | 9.00E−04 | 0.65 |
| IMAGE:731751 | 731751 | 1.00E−03 | 0.57 |
| CLDN1 | 594279 | 1.00E−03 | 0.39 |
| FLJ12604 | 731128 | 1.20E−03 | 0.67 |
| C19orf33 | 379540 | 1.30E−03 | 0.65 |
| SLC6A8 | 725877 | 1.40E−03 | 0.38 |
| IMP-2 | 743774 | 1.50E−03 | 0.52 |
| IMAGE:136976 | 136976 | 1.50E−03 | 0.69 |
| IMAGE:178984 | 178984 | 1.80E−03 | 0.48 |
| ABCC3 | 208097 | 2.60E−03 | 0.55 |
| DAF | 627107 | 3.30E−03 | 0.70 |
| GABRE | 209137 | 3.80E−03 | 0.74 |
| LOC284739 | 741587 | 4.10E−03 | 0.64 |
| SPINL | 236399 | 4.40E−03 | 0.56 |
| IMAGE:234148 | 234148 | 4.70E−03 | 0.70 |
| MYC | 129438 | 4.70E−03 | 0.67 |
| TIAM1 | 23612 | 4.80E−03 | 0.69 |
| ITGAX | 234357 | 5.10E−03 | 0.70 |
| FABP5 | 1088781 | 5.10E−03 | 0.72 |
| DKFZp434M202 | 743118 | 5.40E−03 | 0.76 |
| NOTCH1 | 359461 | 5.50E−03 | 0.75 |
| DF | 666128 | 6.20E−03 | 0.54 |
| APOD | 838611 | 8.70E−03 | 0.49 |
| TCL1A | 200018 | 1.05E−02 | 0.76 |
| CITED1 | 265558 | 1.12E−02 | 0.56 |
| SLAMF8 | 288807 | 1.13E−02 | 0.74 |
| APBA3 | 239086 | 1.14E−02 | 0.28 |
| FLJ22794 | 137454 | 1.18E−02 | 0.76 |
| FLJ12688 | 1735081 | 1.22E−02 | 0.71 |
| DHCR24 | 742965 | 1.23E−02 | 0.64 |
| IGLC1 | 160163 | 1.24E−02 | 0.49 |
| TYROBP | 1146624 | 1.30E−02 | 0.57 |
| RASGRP1 | 725707 | 1.35E−02 | 0.63 |
| CD3G | 110938 | 1.35E−02 | 0.77 |
| HLA-DRA | 153411 | 1.39E−02 | 0.51 |
| DPP4 | 343987 | 1.42E−02 | 0.64 |
| IGKV1-5 | 155345 | 1.42E−02 | 0.43 |
| IGH@ | 289337 | 1.44E−02 | 0.28 |
| CD74 | 117072 | 1.46E−02 | 0.63 |
| HLA-DPA1 | 191881 | 1.51E−02 | 0.51 |
| ALDH1A1 | 309697 | 1.56E−02 | 0.68 |
| MGC21518 | 738315 | 1.70E−02 | 0.71 |
| IMAGE:727496 | 727496 | 1.79E−02 | 0.68 |
| CD79A | 1056782 | 1.82E−02 | 0.71 |
| HCA112 | 486201 | 1.83E−02 | 0.70 |
| LAIR1 | 665566 | 1.84E−02 | 0.79 |
| S100A10 | 119939 | 1.85E−02 | 0.70 |
| DOK5 | 25664 | 1.93E−02 | 0.73 |
| MALT1 | 1420842 | 1.93E−02 | 0.31 |
| IMAGE:725758 | 725758 | 1.94E−02 | 0.29 |
| IMAGE:1129745 | 1129745 | 1.94E−02 | 0.30 |
| IGKC | 713896 | 1.98E−02 | 0.29 |
| S100A1 | 175772 | 1.99E−02 | 0.72 |
| CXCL14 | 345034 | 2.01E−02 | 0.45 |
| FLJ35794 | 364204 | 2.06E−02 | 0.73 |
| FRMD4B | 669564 | 2.06E−02 | 0.73 |
| KIAA1576 | 743426 | 2.07E−02 | 0.79 |
| DKFZp586M1819 | 1072914 | 2.09E−02 | 0.60 |
| IMAGE:78294 | 78294 | 2.11E−02 | 0.69 |
| TPD52 | 743259 | 2.11E−02 | 0.54 |
| COL8A2 | 486204 | 2.20E−02 | 0.77 |
| HMGB2 | 884365 | 2.28E−02 | 0.79 |
| IMAGE:504539 | 504539 | 2.30E−02 | 0.80 |
| SLPI | 378813 | 2.31E−02 | 0.71 |
| IMAGE:745018 | 745018 | 2.38E−02 | 0.64 |
| DUSP24 | 740158 | 2.41E−02 | 0.60 |
| IMAGE:687667 | 687667 | 2.43E−02 | 0.66 |
| C21orf70 | 236386 | 2.44E−02 | 0.32 |
| MTX3 | 741661 | 2.59E−02 | 0.81 |
| STK17A | 562904 | 2.63E−02 | 0.78 |
| NOTCH2 | 1641901 | 2.75E−02 | 0.70 |
| TJP3 | 741919 | 2.76E−02 | 0.74 |
| SPDEF | 1188588 | 2.89E−02 | 0.76 |
| LASP1 | 592598 | 3.07E−02 | 0.66 |
| APOE | 1870594 | 3.22E−02 | 0.67 |
| PDK2 | 114008 | 3.23E−02 | 0.59 |
| URB | 809719 | 3.25E−02 | 0.72 |
| CD58 | 2103105 | 3.26E−02 | 0.79 |
| MGC17299 | 713422 | 3.27E−02 | 0.82 |
| S100A12 | 1705397 | 3.31E−02 | 0.81 |
| IMAGE:1089025 | 1089025 | 3.35E−02 | 0.60 |
| RBBP4 | 705147 | 3.36E−02 | 0.82 |
| SMARCA5 | 730037 | 3.44E−02 | 0.82 |
| CD48 | 667144 | 3.45E−02 | 0.67 |
| NBL1 | 503874 | 3.54E−02 | 0.62 |
| IMAGE:484577 | 484577 | 3.65E−02 | 0.76 |
| KISS1 | 812955 | 3.67E−02 | 0.80 |
| ZFOC1 | 744606 | 3.72E−02 | 0.80 |
| DEK | 1016390 | 3.77E−02 | 0.82 |
| CCDC6 | 487848 | 3.78E−02 | 0.79 |
| IMAGE:251427 | 251427 | 3.93E−02 | 0.79 |
| KRT1 | 1544175 | 4.00E−02 | 0.74 |
| MAP2K4 | 726147 | 4.04E−02 | 0.80 |
| IRF4 | 682207 | 4.04E−02 | 0.77 |
| IMAGE:2364998 | 2364998 | 4.51E−02 | 0.40 |
| RFX2 | 731738 | 4.60E−02 | 0.75 |
| HLA-DPB1 | 725548 | 4.62E−02 | 0.69 |
| CLGN | 1049033 | 4.62E−02 | 0.80 |
| SEMA4D | 210587 | 4.63E−02 | 0.81 |
| CD37 | 1131054 | 4.68E−02 | 0.67 |
| IMAGE:744926 | 744926 | 4.70E−02 | 0.80 |
| FLJ39378 | 724042 | 4.70E−02 | 0.69 |
| FLJ27523 | 743246 | 4.72E−02 | 0.67 |
| C4orf6 | 743212 | 4.72E−02 | 0.77 |
| FOLR1 | 131839 | 4.95E−02 | 0.71 |
| CASP3 | 823680 | 5.01E−02 | 0.77 |
| ISG20 | 740604 | 3.51E−05 | 0.60 |
| IMAGE:487086 | 487086 | 1.52E−04 | 0.67 |
| PCDHB15 | 730593 | 1.06E−03 | 0.74 |
| LHFP | 591534 | 2.24E−03 | 0.74 |
| DKFZp434M202 | 743118 | 5.77E−03 | 0.76 |

TABLE 34

| | FTA-b | Over | |
|---|---|---|---|
| name | cloid | p-value | fold |
| DCI | 667892 | 1.00E−07 | 1.63 |
| SPARC | 267358 | 1.00E−07 | 1.47 |
| USP13 | 666007 | 1.00E−04 | 1.56 |
| ETF1 | 146976 | 1.00E−04 | 1.54 |
| CDH13 | 486510 | 1.00E−04 | 1.38 |
| GRB10 | 564994 | 3.00E−04 | 1.42 |
| PITPNC1 | 364436 | 3.00E−04 | 1.52 |
| GLT8D2 | 365271 | 3.00E−04 | 1.84 |
| CRABP1 | 739193 | 4.00E−04 | 3.17 |
| IMAGE:666315 | 666315 | 4.00E−04 | 1.52 |
| KRT7 | 592276 | 6.00E−04 | 1.52 |
| MGC99813 | 739097 | 6.00E−04 | 2.39 |
| MESDC2 | 713653 | 8.00E−04 | 1.41 |
| CD63 | 125552 | 8.00E−04 | 1.33 |
| PVRL2 | 725364 | 9.00E−04 | 1.37 |
| CDH1 | 214008 | 1.00E−03 | 1.56 |
| LOC340073 | 731404 | 1.00E−03 | 1.46 |
| ID1 | 1087348 | 1.10E−03 | 1.79 |
| DIO1 | 296702 | 1.10E−03 | 1.79 |
| TMED4 | 251250 | 1.10E−03 | 1.52 |
| KDR | 469345 | 1.20E−03 | 1.38 |
| SGSH | 669263 | 1.30E−03 | 1.37 |
| SYN3 | 727056 | 2.40E−03 | 1.34 |
| IMAGE:744385 | 744385 | 2.60E−03 | 1.45 |
| HMG20A | 731277 | 3.10E−03 | 1.54 |
| COX6A1 | 512910 | 3.40E−03 | 1.34 |
| LOC388886 | 34007 | 3.50E−03 | 1.40 |
| IMAGE:744439 | 744439 | 4.30E−03 | 1.30 |
| EMX2 | 365121 | 4.50E−03 | 1.38 |
| VTN | 230126 | 4.90E−03 | 1.49 |
| GOLPH2 | 811582 | 6.40E−03 | 1.29 |
| PBXIP1 | 366042 | 8.30E−03 | 1.39 |
| KIAA0789 | 33621 | 8.40E−03 | 1.40 |
| PH-4 | 730938 | 8.70E−03 | 1.27 |
| IMAGE:665649 | 665649 | 9.50E−03 | 1.32 |
| FLT4 | 668815 | 1.00E−02 | 1.29 |
| TPM2 | 740620 | 1.04E−02 | 1.45 |
| HSPB3 | 502598 | 1.39E−02 | 1.56 |
| CDH16 | 726763 | 1.43E−02 | 1.52 |
| IMAGE:364741 | 364741 | 1.50E−02 | 1.44 |
| KLF1 | 208991 | 1.79E−02 | 1.47 |
| CREB3L2 | 136399 | 2.00E−02 | 1.36 |
| ASGR1 | 25883 | 2.00E−02 | 1.44 |
| IMAGE:136686 | 136686 | 2.06E−02 | 1.31 |
| IMAGE:383718 | 383718 | 2.17E−02 | 1.29 |
| PRUNE | 364324 | 2.28E−02 | 1.28 |
| CHRAC1 | 724276 | 2.32E−02 | 1.24 |
| LOC146909 | 726353 | 2.35E−02 | 1.24 |
| NUP214 | 382612 | 2.37E−02 | 1.31 |
| RAD51L1 | 295412 | 2.45E−02 | 1.40 |
| DNMT3B | 645455 | 2.50E−02 | 1.31 |
| TIMP3 | 501476 | 2.60E−02 | 1.42 |
| FBXW5 | 739576 | 2.77E−02 | 1.25 |
| CKLFSF4 | 143759 | 2.81E−02 | 1.23 |
| POLR2A | 740130 | 2.92E−02 | 1.26 |
| SASH1 | 31120 | 3.07E−02 | 1.28 |
| MT1F | 78353 | 3.55E−02 | 1.54 |
| MGC20781 | 731227 | 3.93E−02 | 1.26 |
| LRRC28 | 26519 | 4.09E−02 | 1.30 |
| NDUFA7 | 364469 | 4.10E−02 | 1.22 |
| GNB4 | 813260 | 4.22E−02 | 1.35 |
| HIS1 | 342551 | 4.30E−02 | 1.27 |
| E2F5 | 809828 | 4.59E−02 | 1.43 |
| GNAI1 | 753215 | 4.63E−02 | 1.25 |
| ADAMTS2 | 364844 | 1.61E−04 | 1.41 |
| GRB10 | 564994 | 1.80E−04 | 1.42 |
| TPM2 | 740620 | 9.35E−03 | 1.45 |
| SPARC | 267358 | 1.09E−04 | 1.47 |
| HAS3 | 667533 | 1.07E−04 | 1.52 |
| IMAGE:666315 | 666315 | 5.63E−04 | 1.52 |
| USP13 | 666007 | 9.96E−05 | 1.56 |
| KRT4 | 1173570 | 2.87E−04 | 1.65 |
| KRTHB5 | 364569 | 1.00E−07 | 1.94 |
| TP53I11 | 667514 | 1.22E−04 | 1.94 |
| METRN | 487475 | 1.00E−07 | 2.15 |

TABLE 35

| | FTC | Under | |
|---|---|---|---|
| name | cloid | p-value | fold |
| LAPTM4A | 726684 | 1.00E−07 | 0.24 |
| TIMP3 | 501476 | 1.00E−07 | 0.29 |
| NRIP1 | 38775 | 2.00E−04 | 0.39 |
| TMED4 | 251250 | 3.00E−04 | 0.28 |
| DEPDC6 | 669318 | 5.00E−04 | 0.39 |
| NTRK3 | 35356 | 7.00E−04 | 0.45 |
| NDUFA7 | 364469 | 9.00E−04 | 0.48 |
| MESDC2 | 713653 | 1.70E−03 | 0.50 |
| CRABP1 | 739193 | 2.10E−03 | 0.13 |
| LRRC28 | 26519 | 2.50E−03 | 0.40 |
| LRP1 | 30219 | 2.60E−03 | 0.40 |
| IGKV1-5 | 155345 | 3.70E−03 | 0.13 |
| ARMCX3 | 251452 | 4.50E−03 | 0.39 |
| IMAGE:739953 | 739953 | 4.70E−03 | 0.05 |
| LILRB2 | 202897 | 5.00E−03 | 0.45 |
| HLA- | 490710 | 5.20E−03 | 0.18 |
| DRB1 | | | |
| MGC99813 | 739097 | 6.20E−03 | 0.23 |
| KDR | 469345 | 6.80E−03 | 0.52 |
| EMX2 | 365121 | 7.80E−03 | 0.51 |
| MDK | 309009 | 7.90E−03 | 0.29 |
| PITPNC1 | 364436 | 7.90E−03 | 0.49 |
| NEK6 | 725345 | 9.10E−03 | 0.52 |
| HLA-DRA | 153411 | 9.30E−03 | 0.21 |
| IMAGE:744897 | 744897 | 9.90E−03 | 0.15 |
| IMAGE:725758 | 725758 | 1.01E−02 | 0.06 |
| RGS19 | 485803 | 1.02E−02 | 0.50 |
| IMAGE:175268 | 175268 | 1.18E−02 | 0.51 |
| LRRC17 | 120649 | 1.25E−02 | 0.37 |
| PPIL4 | 364777 | 1.31E−02 | 0.50 |
| HLA- | 191881 | 1.32E−02 | 0.22 |
| DPA1 | | | |
| IGHM | 276658 | 1.33E−02 | 0.37 |
| MRPS11 | 471574 | 1.40E−02 | 0.50 |
| IMAGE:1129745 | 1129745 | 1.42E−02 | 0.06 |
| PTTG1 | 742935 | 1.45E−02 | 0.43 |
| AIF1 | 343867 | 1.50E−02 | 0.40 |
| IGKC | 713896 | 1.85E−02 | 0.07 |
| C21orf70 | 236386 | 1.94E−02 | 0.07 |
| CD69 | 276727 | 2.02E−02 | 0.55 |
| B2M | 342416 | 2.07E−02 | 0.48 |
| CSF1 | 124554 | 2.09E−02 | 0.53 |
| ISGF3G | 724588 | 2.20E−02 | 0.57 |
| CD81 | 1057773 | 2.63E−02 | 0.60 |
| MALT1 | 1420842 | 2.72E−02 | 0.09 |
| TM7SF3 | 666928 | 2.80E−02 | 0.60 |
| MAD2L2 | 727178 | 2.89E−02 | 0.57 |
| HLA-A | 1013595 | 2.97E−02 | 0.43 |
| HLA- | 725548 | 3.14E−02 | 0.41 |
| DPB1 | | | |
| SLC17A2 | 207920 | 3.18E−02 | 0.37 |
| MRC2 | 235882 | 3.29E−02 | 0.61 |
| HLA-B | 964736 | 3.32E−02 | 0.47 |
| CERKL | 489326 | 3.48E−02 | 0.51 |
| TYROBP | 1146624 | 3.70E−02 | 0.35 |
| CD37 | 1131054 | 3.78E−02 | 0.39 |
| HBA2 | 469647 | 3.83E−02 | 0.28 |
| SLCO3A1 | 725696 | 3.88E−02 | 0.38 |
| C10orf116 | 740941 | 3.97E−02 | 0.44 |
| FLT4 | 668815 | 3.98E−02 | 0.63 |
| S100A12 | 1705397 | 4.10E−02 | 0.64 |
| MGC19604 | 687468 | 4.48E−02 | 0.64 |
| CYP2C9 | 195712 | 4.55E−02 | 0.54 |
| HIS1 | 342551 | 4.78E−02 | 0.59 |
| NCB5OR | 743367 | 4.92E−02 | 0.50 |
| HLA- | 80109 | 5.07E−02 | 0.33 |
| DQA1 | | | |
| CD3Z | 1899942 | 5.12E−02 | 0.65 |
| IMAGE:729489 | 729489 | 5.20E−02 | 0.42 |
| HIST1H4C | 682451 | 1.57E−05 | 0.25 |
| SUCLG2 | 687551 | 7.00E−07 | 0.27 |
| PAI-RBP1 | 236055 | 5.30E−06 | 0.29 |
| GHITM | 25077 | 5.30E−06 | 0.31 |
| CHST3 | 665327 | 2.90E−06 | 0.32 |
| C22orf9 | 667250 | 2.00E−07 | 0.34 |
| ZNF364 | 471834 | 5.80E−06 | 0.39 |
| LRRC28 | 26519 | 1.51E−03 | 0.40 |
| EMX2 | 365121 | 6.95E−03 | 0.51 |

TABLE 36

| | FTC | Over | |
|---|---|---|---|
| name | cloid | p-value | fold |
| NR2F2 | 72744 | 1.00E−07 | 3.11 |
| ZBTB16 | 2467442 | 1.00E−07 | 4.68 |
| IMAGE:726782 | 726782 | 1.00E−07 | 2.43 |
| KIAA1576 | 743426 | 1.00E−04 | 3.33 |
| IMAGE:136801 | 136801 | 1.00E−04 | 2.26 |
| LARS | 135407 | 5.00E−04 | 2.34 |
| DSP | 135975 | 5.00E−04 | 2.53 |
| FLJ22794 | 137454 | 6.00E−04 | 2.39 |
| C9orf72 | 726849 | 7.00E−04 | 2.35 |
| MGC5395 | 238840 | 1.00E−03 | 2.66 |
| CCND1 | 324079 | 1.10E−03 | 2.90 |
| VDAC1 | 486221 | 1.40E−03 | 2.45 |
| ATP8B1 | 669574 | 1.60E−03 | 2.11 |
| MLANA | 266361 | 1.80E−03 | 2.04 |
| LOC286170 | 134858 | 2.40E−03 | 2.33 |
| NAT5 | 731073 | 2.80E−03 | 2.23 |
| NOTCH1 | 359461 | 3.00E−03 | 2.16 |
| ZFOC1 | 744606 | 3.30E−03 | 2.16 |
| MGAT3 | 731060 | 3.50E−03 | 2.04 |
| LATS2 | 666778 | 3.80E−03 | 2.15 |
| IMAGE:726513 | 726513 | 5.70E−03 | 1.77 |
| ELK4 | 236155 | 5.70E−03 | 1.85 |
| IMAGE:744616 | 744616 | 6.30E−03 | 1.85 |
| SH3RF2 | 744797 | 7.60E−03 | 2.18 |
| HSPB1 | 23827 | 9.50E−03 | 2.03 |
| DKFZp434M202 | 743118 | 1.07E−02 | 1.82 |
| BAZ2B | 609631 | 1.10E−02 | 1.78 |
| CKLFSF4 | 143759 | 1.11E−02 | 1.74 |
| PDCD4 | 665376 | 1.21E−02 | 2.37 |
| LOC285961 | 742542 | 1.41E−02 | 1.70 |
| CKS2 | 725454 | 1.61E−02 | 2.14 |
| PDLIM1 | 135689 | 1.64E−02 | 2.19 |
| TM4SF8 | 713647 | 1.69E−02 | 1.75 |
| IMP-2 | 743774 | 1.69E−02 | 2.96 |
| IMAGE:136014 | 136014 | 1.71E−02 | 2.00 |
| IMAGE:724416 | 724416 | 1.73E−02 | 2.26 |
| NFKB1 | 687782 | 1.81E−02 | 1.91 |
| IDH3B | 723755 | 1.85E−02 | 1.76 |
| HP1-BP74 | 726092 | 1.85E−02 | 1.94 |
| FLJ12604 | 731128 | 1.95E−02 | 1.99 |
| DNALI1 | 782688 | 2.13E−02 | 2.10 |
| MLL | 80688 | 2.38E−02 | 1.78 |
| KCNC3 | 53333 | 2.44E−02 | 1.85 |
| LIPT1 | 22085 | 2.55E−02 | 1.66 |
| IMAGE:46991 | 46991 | 2.81E−02 | 1.95 |
| FARP1 | 486708 | 3.07E−02 | 1.76 |
| INHBB | 730012 | 3.67E−02 | 2.18 |
| C22orf16 | 236119 | 3.81E−02 | 1.60 |
| APLP2 | 549054 | 4.59E−02 | 1.82 |
| GFRA3 | 2716748 | 4.52E−03 | 1.93 |
| SH3RF2 | 744797 | 5.37E−03 | 2.18 |
| CEECAM1 | 666784 | 2.67E−04 | 2.22 |
| TIE1 | 743043 | 5.46E−05 | 2.52 |
| MGC5395 | 238840 | 5.07E−04 | 2.66 |
| LRP8 | 415554 | 1.43E−04 | 2.74 |
| IMAGE:729896 | 729896 | 3.00E−07 | 3.22 |

TABLE 37

| | MNG | Under | |
|---|---|---|---|
| name | cloid | p-value | fold |
| GOLPH2 | 811582 | 3.90E−03 | 0.80 |
| KLF4 | 188232 | 6.10E−03 | 0.69 |
| IRF4 | 682207 | 6.20E−03 | 0.73 |
| CASP4 | 356960 | 8.90E−03 | 0.77 |
| ELK4 | 236155 | 9.20E−03 | 0.80 |
| TIMP3 | 501476 | 1.03E−02 | 0.71 |
| SLC9A3R1 | 773286 | 1.41E−02 | 0.77 |
| C1QBP | 173371 | 1.50E−02 | 0.81 |
| CD69 | 276727 | 1.56E−02 | 0.81 |
| SYP | 30471 | 1.75E−02 | 0.73 |
| DNMT3B | 645455 | 1.80E−02 | 0.79 |
| LOC155435 | 745273 | 2.02E−02 | 0.73 |
| IMAGE:744881 | 744881 | 2.03E−02 | 0.79 |
| S100A12 | 1705397 | 2.12E−02 | 0.82 |
| GPI | 741474 | 2.22E−02 | 0.81 |
| IMAGE:667527 | 667527 | 2.47E−02 | 0.77 |
| SLC17A2 | 207920 | 2.56E−02 | 0.67 |
| COX6A1 | 512910 | 2.74E−02 | 0.82 |
| TACC3 | 255398 | 2.86E−02 | 0.82 |
| STAT1 | 110101 | 2.92E−02 | 0.84 |
| SPON2 | 723923 | 2.94E−02 | 0.82 |
| CCL4 | 259552 | 3.11E−02 | 0.69 |
| MYC | 129438 | 3.22E−02 | 0.77 |
| IMAGE:363955 | 363955 | 3.28E−02 | 0.76 |
| MLANA | 266361 | 3.64E−02 | 0.84 |
| MRC2 | 235882 | 3.72E−02 | 0.84 |
| NR2F1 | 253386 | 3.81E−02 | 0.82 |
| LOC284669 | 364885 | 3.93E−02 | 0.83 |
| IMAGE:740105 | 740105 | 4.06E−02 | 0.84 |
| IL16 | 204381 | 4.32E−02 | 0.80 |
| IMAGE:742837 | 742837 | 4.46E−02 | 0.83 |
| NOMO1 | 134615 | 4.64E−02 | 0.85 |
| PPP3CB | 1116948 | 4.68E−02 | 0.81 |
| IMAGE:136801 | 136801 | 5.03E−02 | 0.86 |
| NR4A1 | 1073288 | 1.88E−03 | 0.64 |
| SLC17A2 | 207920 | 2.70E−02 | 0.67 |
| IMAGE:731292 | 731292 | 2.65E−02 | 0.68 |
| PRKCA | 768246 | 9.12E−04 | 0.71 |
| CALM1 | 594510 | 1.21E−03 | 0.77 |
| IMAGE:745187 | 745187 | 1.15E−03 | 0.78 |

TABLE 38

| | MNG | Over | |
|---|---|---|---|
| name | cloid | p-value | fold |
| RODH | 471641 | 1.00E−07 | 2.57 |
| MGC17299 | 713422 | 1.00E−04 | 1.37 |
| C10orf116 | 740941 | 2.00E−04 | 1.72 |
| NR2F2 | 72744 | 3.00E−04 | 1.40 |
| CAV1 | 309645 | 3.00E−04 | 1.59 |
| IMAGE:744505 | 744505 | 4.00E−04 | 1.38 |
| IMAGE:731726 | 731726 | 4.00E−04 | 1.52 |
| IMAGE:726782 | 726782 | 6.00E−04 | 1.38 |
| PDLIM1 | 135689 | 8.00E−04 | 1.48 |
| TTC17 | 665668 | 1.10E−03 | 1.31 |
| LRP2 | 2055272 | 1.30E−03 | 1.41 |
| FOXO1A | 151247 | 1.60E−03 | 1.30 |
| C20orf19 | 366032 | 1.80E−03 | 1.31 |
| EPHB6 | 172982 | 2.00E−03 | 1.58 |
| DKFZp434M202 | 743118 | 2.10E−03 | 1.32 |
| PRUNE | 364324 | 2.50E−03 | 1.32 |
| ANKRD20B | 745106 | 4.30E−03 | 1.40 |
| ALCAM | 172828 | 5.60E−03 | 1.24 |
| ISG20 | 740604 | 9.50E−03 | 1.33 |
| IMAGE:744055 | 744055 | 9.50E−03 | 1.48 |
| FCGBP | 154172 | 9.80E−03 | 1.59 |
| FOLR1 | 131839 | 1.02E−02 | 1.48 |
| KCNC3 | 53333 | 1.04E−02 | 1.29 |
| PLA2G6 | 744087 | 1.07E−02 | 1.32 |
| IMAGE:726513 | 726513 | 1.27E−02 | 1.22 |
| RAD51L1 | 295412 | 1.65E−02 | 1.36 |
| LOC286170 | 134858 | 1.73E−02 | 1.28 |
| LRP1 | 30219 | 1.78E−02 | 1.28 |
| FGFRL1 | 739956 | 1.90E−02 | 1.32 |
| FOXO3A | 109142 | 1.97E−02 | 1.21 |
| FARP1 | 486708 | 2.03E−02 | 1.25 |
| MT1F | 78353 | 2.37E−02 | 1.50 |
| SLC6A8 | 725877 | 2.38E−02 | 1.78 |
| C9orf72 | 726849 | 2.44E−02 | 1.23 |
| DNALI1 | 782688 | 2.55E−02 | 1.31 |
| CXCL14 | 345034 | 2.71E−02 | 1.95 |
| ASGR1 | 25883 | 2.89E−02 | 1.34 |
| ZBTB16 | 2467442 | 3.04E−02 | 1.38 |
| RPS6KA2 | 22711 | 3.27E−02 | 1.28 |
| IMP-2 | 743774 | 3.94E−02 | 1.44 |
| SLC25A23 | 741954 | 3.95E−02 | 1.26 |
| ZFOC1 | 744606 | 4.04E−02 | 1.20 |
| PLXNB2 | 1420676 | 4.11E−02 | 1.26 |
| C4A | 724366 | 4.19E−02 | 1.46 |
| IMAGE:1716286 | 1716286 | 4.58E−02 | 1.32 |
| MLL | 80688 | 4.74E−02 | 1.21 |
| SPINL | 236399 | 4.87E−02 | 1.42 |
| UBE1L | 250883 | 4.89E−02 | 1.23 |
| DKFZp761P0423 | 730829 | 5.23E−02 | 1.27 |
| IMAGE:250463 | 250463 | 6.76E−03 | 1.29 |
| TTC17 | 665668 | 5.24E−04 | 1.31 |
| ZNF83 | 486356 | 3.72E−04 | 1.32 |
| CNTN6 | 257773 | 2.34E−04 | 1.38 |
| IMAGE:726782 | 726782 | 3.97E−04 | 1.38 |
| IMAGE:744505 | 744505 | 2.51E−04 | 1.38 |
| PDLIM1 | 135689 | 9.76E−04 | 1.48 |
| IMAGE:731726 | 731726 | 3.90E−04 | 1.52 |
| FLJ11127 | 668625 | 2.30E−06 | 1.54 |
| CAV1 | 309645 | 3.38E−04 | 1.59 |
| C10orf116 | 740941 | 2.82E−04 | 1.72 |

TABLE 39

| | OTA | Under | |
|---|---|---|---|
| name | cloid | p-value | fold |
| MLL | 80688 | 1.00E−07 | 0.55 |
| DCN | 666410 | 1.00E−07 | 0.50 |
| SLC6A8 | 725877 | 1.00E−07 | 0.23 |
| SNRP70 | 729971 | 1.00E−07 | 0.52 |
| SPINL | 236399 | 1.00E−07 | 0.38 |
| HP1-BP74 | 726092 | 1.00E−07 | 0.59 |
| C4A | 724366 | 1.00E−07 | 0.39 |
| EPHB6 | 172982 | 1.00E−07 | 0.48 |
| APOE | 1870594 | 1.00E−07 | 0.45 |
| LGALS3BP | 742100 | 1.00E−07 | 0.48 |
| PSAT1 | 366388 | 1.00E−07 | 0.66 |
| PBXIP1 | 366042 | 1.00E−07 | 0.60 |
| ZNF395 | 744983 | 1.00E−07 | 0.66 |
| HNRPA3 | 365349 | 1.00E−07 | 0.68 |
| LOC286076 | 731275 | 1.00E−07 | 0.66 |
| NFIB | 416959 | 1.00E−07 | 0.67 |
| KCNC3 | 53333 | 1.00E−07 | 0.63 |
| FOXO1A | 151247 | 1.00E−07 | 0.66 |
| IMAGE:731726 | 731726 | 1.00E−07 | 0.57 |
| CXCL14 | 345034 | 1.00E−07 | 0.25 |
| NUP214 | 382612 | 1.00E−07 | 0.63 |
| URB | 809719 | 1.00E−07 | 0.55 |
| RFX2 | 731738 | 1.00E−07 | 0.57 |
| INHBB | 730012 | 1.00E−07 | 0.55 |
| ITPR1 | 667348 | 1.00E−07 | 0.66 |
| CaMKIINalpha | 173820 | 1.00E−07 | 0.66 |
| RIPK3 | 667313 | 1.00E−07 | 0.68 |
| SF3B1 | 739247 | 1.00E−07 | 0.68 |
| FLJ22794 | 137454 | 1.00E−07 | 0.67 |
| PLA2G6 | 744087 | 1.00E−07 | 0.62 |
| SPATA12 | 730300 | 1.00E−07 | 0.69 |
| HMGB2 | 884365 | 1.00E−07 | 0.68 |
| PDLIM1 | 135689 | 1.00E−07 | 0.60 |
| IMP-2 | 743774 | 1.00E−07 | 0.47 |
| RASGRP1 | 725707 | 1.00E−07 | 0.52 |
| LIPT1 | 22085 | 1.00E−07 | 0.70 |
| TLX3 | 1860115 | 1.00E−07 | 0.59 |
| IL19 | 1932168 | 1.00E−07 | 0.62 |
| UBE1L | 250883 | 1.00E−07 | 0.66 |
| C9orf72 | 726849 | 1.00E−07 | 0.69 |
| FRMD4B | 669564 | 1.00E−07 | 0.63 |
| IMAGE:2266583 | 2266583 | 1.00E−07 | 0.70 |
| PDGFRL | 139242 | 1.00E−07 | 0.63 |
| C10orf116 | 740941 | 1.00E−07 | 0.54 |
| 37500 | 729542 | 1.00E−07 | 0.61 |
| PRUNE | 364324 | 1.00E−07 | 0.69 |
| PDCD4 | 665376 | 1.00E−07 | 0.65 |
| CFH | 665784 | 1.00E−07 | 0.53 |
| ESRRBL1 | 51232 | 1.00E−07 | 0.70 |
| IMAGE:731751 | 731751 | 1.00E−07 | 0.57 |
| IMAGE:175268 | 175268 | 1.00E−07 | 0.68 |
| TAPBP | 723753 | 1.00E−07 | 0.73 |
| BAZ2B | 609631 | 1.00E−07 | 0.75 |
| MGC5395 | 238840 | 1.00E−07 | 0.67 |
| SGSH | 669263 | 1.00E−07 | 0.73 |
| FLJ12592 | 252489 | 1.00E−07 | 0.75 |
| FARP1 | 486708 | 1.00E−07 | 0.71 |
| PTPRS | 666066 | 1.00E−07 | 0.76 |
| DKFZp761P0423 | 730829 | 1.00E−07 | 0.65 |
| KIAA0789 | 33621 | 1.00E−07 | 0.70 |
| TIAM1 | 23612 | 1.00E−04 | 0.61 |
| G3BP | 667239 | 1.00E−04 | 0.70 |
| TPM2 | 740620 | 1.00E−04 | 0.63 |
| FLJ12604 | 731128 | 1.00E−04 | 0.69 |
| FGFRL1 | 739956 | 1.00E−04 | 0.67 |
| CICE | 725791 | 1.00E−04 | 0.74 |
| LAPTM4A | 726684 | 1.00E−04 | 0.69 |
| ZNF251 | 486401 | 2.00E−04 | 0.51 |
| HSPB1 | 23827 | 2.00E−04 | 0.70 |
| GLT8D2 | 365271 | 2.00E−04 | 0.61 |
| EMX2 | 365121 | 2.00E−04 | 0.73 |
| LOC286170 | 134858 | 2.00E−04 | 0.72 |
| DNALI1 | 782688 | 3.00E−04 | 0.66 |
| IMAGE:741835 | 741835 | 3.00E−04 | 0.74 |
| MGP | 590264 | 3.00E−04 | 0.59 |
| RODH | 471641 | 3.00E−04 | 0.49 |
| IMAGE:738332 | 738332 | 4.00E−04 | 0.57 |
| COL27A1 | 379517 | 4.00E−04 | 0.76 |
| SALL4 | 726454 | 5.00E−04 | 0.53 |
| IMAGE:726782 | 726782 | 5.00E−04 | 0.74 |
| LOC340073 | 731404 | 5.00E−04 | 0.73 |
| LILRA1 | 2032639 | 5.00E−04 | 0.75 |
| SLC25A23 | 741954 | 5.00E−04 | 0.72 |
| PLAC9 | 366115 | 5.00E−04 | 0.68 |
| APOL3 | 366289 | 6.00E−04 | 0.67 |
| IMAGE:727154 | 727154 | 6.00E−04 | 0.74 |
| LOC440712 | 725268 | 7.00E−04 | 0.73 |
| POLR2A | 740130 | 7.00E−04 | 0.76 |
| ANKRD20B | 745106 | 7.00E−04 | 0.71 |
| PET112L | 743125 | 8.00E−04 | 0.76 |
| TTC17 | 665668 | 8.00E−04 | 0.79 |
| APLP2 | 549054 | 1.00E−03 | 0.70 |
| CA11 | 282587 | 1.00E−03 | 0.78 |
| ETF1 | 146976 | 1.10E−03 | 0.76 |
| FOXO3A | 109142 | 1.20E−03 | 0.79 |
| NR2F1 | 253386 | 1.20E−03 | 0.76 |
| IL11RA | 1101773 | 1.30E−03 | 0.74 |
| FOLR1 | 131839 | 1.40E−03 | 0.64 |
| CDH1 | 214008 | 1.40E−03 | 0.73 |
| IQCA | 669510 | 1.60E−03 | 0.78 |
| IMAGE:665833 | 665833 | 1.60E−03 | 0.78 |
| TIMP1 | 162246 | 1.60E−03 | 0.57 |
| DKFZp434M202 | 743118 | 1.60E−03 | 0.78 |
| DJ462O23.2 | 738970 | 1.80E−03 | 0.79 |
| F10 | 310519 | 1.80E−03 | 0.79 |
| IMAGE:742853 | 742853 | 2.00E−03 | 0.67 |
| IMAGE:726106 | 726106 | 2.00E−03 | 0.76 |
| LRP1 | 30219 | 2.10E−03 | 0.74 |
| LOC126669 | 682088 | 2.30E−03 | 0.77 |
| ISGF3G | 724588 | 2.60E−03 | 0.77 |
| IMAGE:745125 | 745125 | 2.80E−03 | 0.77 |
| FLJ35794 | 364204 | 2.90E−03 | 0.74 |
| ALCAM | 172828 | 3.20E−03 | 0.81 |
| USP13 | 666007 | 3.40E−03 | 0.76 |
| IMAGE:727496 | 727496 | 3.40E−03 | 0.68 |
| CHTF18 | 740971 | 3.70E−03 | 0.80 |
| CASP3 | 823680 | 3.70E−03 | 0.74 |
| CCL19 | 1707527 | 3.90E−03 | 0.49 |
| TSHR | 565317 | 3.90E−03 | 0.73 |
| NOTCH2 | 1641901 | 4.10E−03 | 0.70 |
| CAV1 | 309645 | 4.10E−03 | 0.71 |
| DF | 666128 | 4.10E−03 | 0.61 |
| CD81 | 1057773 | 4.20E−03 | 0.80 |
| LOC400451 | 667174 | 4.40E−03 | 0.76 |
| SFRP4 | 285693 | 4.50E−03 | 0.64 |
| MDK | 309009 | 4.80E−03 | 0.64 |
| TPD52 | 743259 | 4.80E−03 | 0.55 |
| LATS2 | 666778 | 4.90E−03 | 0.79 |
| IMAGE:731152 | 731152 | 5.00E−03 | 0.71 |
| PREB | 740347 | 5.20E−03 | 0.61 |
| HDAC1 | 1896337 | 5.20E−03 | 0.78 |
| APOL1 | 665632 | 5.20E−03 | 0.62 |
| C19orf33 | 379540 | 5.40E−03 | 0.75 |
| CAPN3 | 757248 | 5.40E−03 | 0.82 |
| KCNK5 | 134978 | 5.90E−03 | 0.76 |
| PLXNB2 | 1420676 | 6.40E−03 | 0.76 |
| SEMA4D | 210587 | 6.60E−03 | 0.80 |
| ATP6V1G2 | 726424 | 6.60E−03 | 0.77 |
| LASP1 | 592598 | 6.60E−03 | 0.66 |
| CD1C | 665881 | 7.70E−03 | 0.81 |
| FOXQ1 | 725511 | 7.80E−03 | 0.74 |
| IMAGE:738896 | 738896 | 7.80E−03 | 0.55 |
| C9orf97 | 740885 | 7.90E−03 | 0.64 |
| RAD51L1 | 295412 | 8.10E−03 | 0.73 |
| NFKB1 | 687782 | 8.40E−03 | 0.80 |
| TNFRSF1B | 73703 | 8.60E−03 | 0.82 |
| LOC203076 | 744756 | 8.60E−03 | 0.81 |
| SLAMF8 | 288807 | 8.90E−03 | 0.78 |
| GIMAP5 | 180259 | 8.90E−03 | 0.80 |
| GPC3 | 137131 | 9.00E−03 | 0.80 |
| APOD | 838611 | 9.10E−03 | 0.56 |
| FLJ39378 | 724042 | 9.30E−03 | 0.68 |
| IMAGE:744366 | 744366 | 9.30E−03 | 0.72 |
| DHRS6 | 364412 | 9.40E−03 | 0.80 |
| CFLAR | 427786 | 9.70E−03 | 0.59 |
| 38596 | 448163 | 9.80E−03 | 0.74 |
| LOC388886 | 34007 | 9.80E−03 | 0.79 |
| HCG4P6 | 325008 | 9.90E−03 | 0.78 |
| DAXX | 742977 | 9.90E−03 | 0.82 |
| VCAM1 | 471101 | 1.00E−02 | 0.77 |
| LYZ | 1256485 | 1.02E−02 | 0.66 |
| SMARCA5 | 730037 | 1.03E−02 | 0.82 |
| IMAGE:742696 | 742696 | 1.16E−02 | 0.53 |
| AQP11 | 713831 | 1.17E−02 | 0.74 |
| ZBTB33 | 725622 | 1.19E−02 | 0.60 |
| GPR124 | 486493 | 1.22E−02 | 0.83 |
| FAM12B | 744413 | 1.31E−02 | 0.74 |
| CHRAC1 | 724276 | 1.35E−02 | 0.83 |
| KRT18 | 725096 | 1.36E−02 | 0.81 |
| IMAGE:234148 | 234148 | 1.38E−02 | 0.78 |
| MDM2 | 239951 | 1.39E−02 | 0.82 |
| IMAGE:134034 | 134034 | 1.40E−02 | 0.77 |
| S100A4 | 868577 | 1.45E−02 | 0.66 |
| IFITM3 | 713623 | 1.48E−02 | 0.80 |
| LOH12CR2 | 366074 | 1.48E−02 | 0.80 |
| SELL | 149910 | 1.50E−02 | 0.82 |
| IMAGE:1877668 | 1877668 | 1.51E−02 | 0.82 |
| FCGBP | 154172 | 1.59E−02 | 0.67 |
| IGH@ | 289337 | 1.73E−02 | 0.37 |
| IMAGE:726513 | 726513 | 1.75E−02 | 0.84 |
| STAT2 | 2306096 | 1.77E−02 | 0.84 |
| PP35 | 726904 | 1.78E−02 | 0.61 |
| GFM2 | 364959 | 1.80E−02 | 0.83 |
| CD9 | 727251 | 1.87E−02 | 0.84 |
| IMAGE:744505 | 744505 | 1.92E−02 | 0.83 |
| NEDD9 | 667602 | 1.93E−02 | 0.64 |
| IMAGE:342256 | 342256 | 2.02E−02 | 0.59 |
| NCK1 | 1326169 | 2.10E−02 | 0.79 |
| IMAGE:744055 | 744055 | 2.18E−02 | 0.74 |
| TLN1 | 187482 | 2.22E−02 | 0.80 |
| CXCR6 | 196115 | 2.23E−02 | 0.82 |
| MGAT3 | 731060 | 2.24E−02 | 0.83 |
| B2M | 342416 | 2.24E−02 | 0.79 |
| IMAGE:743237 | 743237 | 2.31E−02 | 0.75 |
| IFNAR2 | 123950 | 2.33E−02 | 0.82 |
| CD63 | 125552 | 2.62E−02 | 0.85 |
| NBL1 | 503874 | 2.77E−02 | 0.67 |
| GATA4 | 781738 | 2.79E−02 | 0.83 |
| CXCL9 | 1654256 | 2.79E−02 | 0.65 |
| FLJ27523 | 743246 | 2.82E−02 | 0.70 |
| ISG20 | 740604 | 2.83E−02 | 0.81 |
| IMAGE:739953 | 739953 | 2.85E−02 | 0.40 |
| IMAGE:744911 | 744911 | 2.88E−02 | 0.74 |
| FMO5 | 364526 | 2.91E−02 | 0.82 |
| VEGFC | 503189 | 2.95E−02 | 0.84 |
| KIAA1196 | 738938 | 2.96E−02 | 0.83 |
| ABCC3 | 208097 | 2.96E−02 | 0.72 |
| ASGR1 | 25883 | 3.06E−02 | 0.77 |
| RARRES3 | 324225 | 3.20E−02 | 0.73 |
| RANBP2 | 245426 | 3.27E−02 | 0.68 |
| SPON2 | 723923 | 3.36E−02 | 0.83 |
| SARS | 248811 | 3.39E−02 | 0.77 |
| MGC21518 | 738315 | 3.43E−02 | 0.78 |
| IMAGE:743722 | 743722 | 3.45E−02 | 0.83 |
| HLA-A | 1013595 | 3.58E−02 | 0.76 |
| IMAGE:1737165 | 1737165 | 3.83E−02 | 0.76 |
| IMAGE:727173 | 727173 | 3.83E−02 | 0.84 |
| MGC40042 | 744755 | 3.86E−02 | 0.65 |
| GSS | 140405 | 4.00E−02 | 0.82 |
| BCL2L11 | 300194 | 4.09E−02 | 0.86 |
| IMAGE:744468 | 744468 | 4.22E−02 | 0.77 |
| IMAGE:2361911 | 2361911 | 4.36E−02 | 0.64 |
| IMAGE:46991 | 46991 | 4.50E−02 | 0.81 |
| CD38 | 1352408 | 4.64E−02 | 0.80 |
| IMAGE:1129745 | 1129745 | 4.68E−02 | 0.43 |
| IMAGE:1089025 | 1089025 | 4.94E−02 | 0.68 |
| MTMR9 | 745223 | 4.97E−02 | 0.64 |
| IGKC | 713896 | 5.13E−02 | 0.43 |
| SLC6A8 | 725877 | 1.00E−07 | 0.23 |
| UBA52 | 530069 | 4.40E−06 | 0.29 |
| C4A | 724366 | 1.00E−07 | 0.39 |
| DCN | 666410 | 1.00E−07 | 0.50 |
| SNRP70 | 729971 | 1.00E−07 | 0.52 |
| C9orf72 | 726849 | 4.40E−06 | 0.69 |

TABLE 40

| | OTA | Over | |
|---|---|---|---|
| name | cloid | p-value | fold |
| CLECSF12 | 258865 | 1.00E−07 | 3.51 |
| IMAGE:485104 | 485104 | 1.00E−07 | 2.25 |
| PROZ | 430471 | 1.00E−07 | 2.76 |
| OBSCN | 730926 | 1.00E−07 | 1.55 |
| IMAGE:731616 | 731616 | 1.00E−07 | 2.42 |
| HSPC159 | 365045 | 1.00E−07 | 1.70 |
| CHRDL2 | 485872 | 1.00E−07 | 1.58 |
| DC12 | 724895 | 1.00E−07 | 1.71 |
| KCNQ2 | 179534 | 1.00E−07 | 1.77 |
| ALDH1A3 | 486189 | 1.00E−07 | 1.79 |
| SLC36A3 | 731115 | 1.00E−07 | 1.51 |
| OSR1 | 364686 | 1.00E−07 | 1.64 |
| FOXN4 | 731076 | 1.00E−07 | 1.61 |
| BIC | 743270 | 1.00E−07 | 1.65 |
| RPL27A | 178255 | 1.00E−07 | 2.33 |
| SIAT8B | 33133 | 1.00E−07 | 1.50 |
| PRKACB | 366820 | 1.00E−07 | 1.63 |
| KIAA1754L | 365056 | 1.00E−07 | 1.71 |
| ATP11B | 666334 | 1.00E−07 | 1.82 |
| OAS1 | 666703 | 1.00E−07 | 1.87 |
| RORA | 1086823 | 1.00E−07 | 1.63 |
| IMAGE:364741 | 364741 | 1.00E−07 | 1.90 |
| IMAGE:666794 | 666794 | 1.00E−07 | 1.71 |
| LOC56251 | 137478 | 1.00E−07 | 1.50 |
| IMAGE:177857 | 177857 | 1.00E−07 | 1.74 |
| SAFB | 42280 | 1.00E−07 | 1.81 |
| PCSK6 | 31924 | 1.00E−07 | 1.76 |
| BAK1 | 235938 | 1.00E−07 | 1.53 |
| IMAGE:2484270 | 2484270 | 1.00E−07 | 1.62 |
| IMAGE:213529 | 213529 | 1.00E−07 | 1.53 |
| SCMH1 | 724014 | 1.00E−07 | 2.02 |
| NTRK2 | 2048801 | 1.00E−07 | 1.99 |
| ARHGAP10 | 731380 | 1.00E−07 | 1.83 |
| SIGLEC7 | 743331 | 1.00E−07 | 1.50 |
| C21orf84 | 730814 | 1.00E−07 | 1.68 |
| PLCG1 | 1174287 | 1.00E−07 | 1.65 |
| FLJ25067 | 667252 | 1.00E−07 | 2.08 |
| MGC23909 | 731598 | 1.00E−07 | 1.96 |
| SYCP2 | 136863 | 1.00E−07 | 1.77 |
| ULK4 | 744895 | 1.00E−07 | 1.52 |
| ANP32E | 382738 | 1.00E−07 | 1.76 |
| SIAT8F | 667110 | 1.00E−07 | 1.74 |
| MRPS11 | 471574 | 1.00E−07 | 1.59 |
| IGHMBP2 | 23876 | 1.00E−07 | 1.62 |
| MEOX1 | 760065 | 1.00E−07 | 1.67 |
| COX5A | 1085884 | 1.00E−07 | 1.50 |
| IMAGE:745072 | 745072 | 1.00E−07 | 1.76 |
| BIN1 | 2384812 | 1.00E−07 | 1.47 |
| ME3 | 724238 | 1.00E−07 | 1.80 |
| RNF32 | 731422 | 1.00E−07 | 1.91 |
| IMAGE:744899 | 744899 | 1.00E−07 | 1.46 |
| IMAGE:666279 | 666279 | 1.00E−07 | 1.85 |
| CASP5 | 341763 | 1.00E−07 | 1.95 |
| MCC | 731305 | 1.00E−07 | 1.96 |
| C20orf18 | 665433 | 1.00E−07 | 1.83 |
| IMAGE:740105 | 740105 | 1.00E−07 | 1.46 |
| C22orf16 | 236119 | 1.00E−07 | 1.45 |
| SYP | 30471 | 1.00E−07 | 1.75 |
| IMAGE:384844 | 384844 | 1.00E−07 | 1.55 |
| DEFB1 | 665086 | 1.00E−07 | 1.92 |
| LOC284669 | 364885 | 1.00E−07 | 1.48 |
| MAPRE2 | 383868 | 1.00E−07 | 1.92 |
| MRPL45 | 364717 | 1.00E−07 | 1.57 |
| TRIM8 | 714498 | 1.00E−07 | 1.43 |
| IMAGE:744074 | 744074 | 1.00E−07 | 2.10 |
| COX6A1 | 512910 | 1.00E−07 | 1.46 |
| DAB1 | 382621 | 1.00E−07 | 1.45 |
| IMAGE:665649 | 665649 | 1.00E−07 | 1.48 |
| IMAGE:379937 | 379937 | 1.00E−07 | 1.59 |
| APIN | 364706 | 1.00E−07 | 1.56 |
| NME7 | 743982 | 1.00E−07 | 2.08 |
| IMAGE:743619 | 743619 | 1.00E−07 | 1.46 |
| LRPAP1 | 587186 | 1.00E−07 | 1.54 |
| S100A12 | 1705397 | 1.00E−07 | 1.42 |
| GPI | 741474 | 1.00E−07 | 1.47 |
| CASP10 | 241481 | 1.00E−07 | 1.72 |
| JAK1 | 2030501 | 1.00E−07 | 1.52 |
| F5 | 433155 | 1.00E−07 | 1.55 |
| VTN | 230126 | 1.00E−07 | 1.67 |
| JUP | 35628 | 1.00E−07 | 1.84 |
| IMAGE:363955 | 363955 | 1.00E−07 | 1.73 |
| BAP1 | 1012990 | 1.00E−07 | 1.60 |
| LILRB5 | 71428 | 1.00E−07 | 1.56 |
| ALDH3A1 | 525221 | 1.00E−07 | 1.60 |
| MUTED | 731202 | 1.00E−07 | 1.65 |
| IMAGE:667527 | 667527 | 1.00E−07 | 1.63 |
| ZNF319 | 364729 | 1.00E−07 | 2.29 |
| IMAGE:743603 | 743603 | 1.00E−07 | 1.44 |
| C1QBP | 173371 | 1.00E−07 | 1.41 |
| GNAI1 | 753215 | 1.00E−07 | 1.48 |
| FBXO46 | 471664 | 1.00E−07 | 1.53 |
| IMAGE:742837 | 742837 | 1.00E−07 | 1.45 |
| DEXI | 668186 | 1.00E−07 | 1.37 |
| MGC19604 | 687468 | 1.00E−07 | 1.40 |
| GALNT7 | 381854 | 1.00E−07 | 1.52 |
| REV1L | 738916 | 1.00E−07 | 1.93 |
| TFF1 | 1075949 | 1.00E−07 | 1.75 |
| PLCG2 | 201467 | 1.00E−07 | 1.50 |
| LRRIQ2 | 487152 | 1.00E−07 | 1.40 |
| HRK | 767779 | 1.00E−07 | 1.69 |
| GAPD | 152847 | 1.00E−07 | 1.54 |
| SDHA | 40304 | 1.00E−07 | 1.39 |
| PCGF4 | 740457 | 1.00E−07 | 1.62 |
| VDAC1 | 486221 | 1.00E−07 | 1.48 |
| GNAS | 382791 | 1.00E−07 | 1.71 |
| RHOD | 591907 | 1.00E−07 | 1.36 |
| COX7B | 566862 | 1.00E−07 | 1.48 |
| PPIL4 | 364777 | 1.00E−07 | 1.41 |
| IMAGE:744616 | 744616 | 1.00E−07 | 1.34 |
| PTTG1 | 742935 | 1.00E−07 | 1.51 |
| PARD3 | 724642 | 1.00E−04 | 1.42 |
| IMAGE:257555 | 257555 | 1.00E−04 | 1.60 |
| CLGN | 1049033 | 1.00E−04 | 1.46 |
| IMAGE:667258 | 667258 | 1.00E−04 | 1.73 |
| IMAGE:382521 | 382521 | 1.00E−04 | 1.54 |
| AURKC | 731021 | 1.00E−04 | 1.53 |
| FLJ11184 | 384404 | 1.00E−04 | 1.61 |
| IMAGE:742061 | 742061 | 1.00E−04 | 1.46 |
| SIAT8C | 382069 | 1.00E−04 | 1.39 |
| HMG20A | 731277 | 1.00E−04 | 1.57 |
| IMAGE:727289 | 727289 | 1.00E−04 | 1.64 |
| C6orf176 | 173200 | 1.00E−04 | 1.31 |
| MAD2L2 | 727178 | 1.00E−04 | 1.38 |
| FKBP6 | 795736 | 1.00E−04 | 1.42 |
| SLC6A1 | 177967 | 2.00E−04 | 1.35 |
| FLJ23577 | 668452 | 2.00E−04 | 1.70 |
| SIAT7B | 823590 | 2.00E−04 | 1.41 |
| MAK | 382002 | 2.00E−04 | 1.43 |
| MFAP3L | 726821 | 2.00E−04 | 1.29 |
| TOSO | 813174 | 2.00E−04 | 1.50 |
| MGC45840 | 725231 | 3.00E−04 | 1.36 |
| C6orf216 | 731742 | 4.00E−04 | 1.39 |
| MGC20781 | 731227 | 4.00E−04 | 1.40 |
| CD33 | 1917430 | 4.00E−04 | 1.52 |
| FSHPRH1 | 667355 | 5.00E−04 | 1.32 |
| CDS1 | 135630 | 5.00E−04 | 1.40 |
| IMAGE:383718 | 383718 | 5.00E−04 | 1.35 |
| CCDC6 | 487848 | 6.00E−04 | 1.36 |
| IMAGE:729875 | 729875 | 6.00E−04 | 1.34 |
| TH | 813654 | 7.00E−04 | 1.29 |
| DUSP24 | 740158 | 8.00E−04 | 1.80 |
| MATN1 | 1624260 | 1.10E−03 | 1.38 |
| SLC9A3R1 | 773286 | 1.10E−03 | 1.36 |
| ABCA8 | 284828 | 1.50E−03 | 1.39 |
| PINK1 | 729929 | 1.60E−03 | 1.39 |
| NAT5 | 731073 | 1.60E−03 | 1.29 |
| WDR5 | 731023 | 1.80E−03 | 1.25 |
| IMAGE:136686 | 136686 | 2.10E−03 | 1.34 |
| NIFIE14 | 472160 | 2.10E−03 | 1.26 |
| IMAGE:484577 | 484577 | 2.40E−03 | 1.37 |
| RASAL2 | 486304 | 2.50E−03 | 1.61 |
| IFI30 | 740931 | 2.50E−03 | 1.60 |
| CD3D | 377560 | 2.60E−03 | 1.39 |
| DOK5 | 25664 | 2.70E−03 | 1.38 |
| FLJ20989 | 669557 | 2.90E−03 | 1.72 |
| BST2 | 811024 | 3.00E−03 | 1.28 |
| SPI1 | 1285305 | 3.80E−03 | 1.25 |
| CCL11 | 502658 | 4.30E−03 | 1.32 |
| CD69 | 276727 | 4.70E−03 | 1.26 |
| ELK4 | 236155 | 5.00E−03 | 1.25 |
| TM7SF3 | 666928 | 6.30E−03 | 1.24 |
| LAMB3 | 1103402 | 6.60E−03 | 1.29 |
| IMAGE:682585 | 682585 | 6.80E−03 | 1.24 |
| TRAF2 | 966894 | 6.90E−03 | 1.24 |
| NOMO1 | 134615 | 6.90E−03 | 1.21 |
| AMICA1 | 487436 | 9.30E−03 | 1.30 |
| DIO1 | 296702 | 9.40E−03 | 1.44 |
| CBL | 1578721 | 1.02E−02 | 1.30 |
| RGS19 | 485803 | 1.14E−02 | 1.25 |
| KLK2 | 1102600 | 1.16E−02 | 1.23 |
| NDUFA7 | 364469 | 1.17E−02 | 1.21 |
| FBXW5 | 739576 | 1.29E−02 | 1.22 |
| LOC285961 | 742542 | 1.39E−02 | 1.19 |
| ICOSLG | 2074228 | 1.52E−02 | 1.23 |
| IMAGE:743517 | 743517 | 1.61E−02 | 1.19 |
| LEFTY2 | 340657 | 2.07E−02 | 1.26 |
| ICAM3 | 156183 | 2.15E−02 | 1.20 |
| FCHO1 | 742763 | 2.30E−02 | 1.19 |
| GFPT2 | 485085 | 2.32E−02 | 1.23 |
| VAV1 | 80384 | 2.74E−02 | 1.38 |
| MLANA | 266361 | 2.75E−02 | 1.18 |
| ARVCF | 40633 | 3.27E−02 | 1.17 |
| S100A1 | 175772 | 3.28E−02 | 1.27 |
| IMAGE:666315 | 666315 | 3.67E−02 | 1.23 |
| SH3RF2 | 744797 | 4.29E−02 | 1.22 |
| TBC1D2 | 236331 | 4.42E−02 | 1.19 |
| HCNGP | 365934 | 1.92E−05 | 1.40 |
| GNAI1 | 753215 | 8.90E−06 | 1.48 |
| OBSCN | 730926 | 1.00E−07 | 1.55 |
| CHRDL2 | 485872 | 1.00E−07 | 1.58 |
| HSPC159 | 365045 | 1.00E−07 | 1.70 |
| DC12 | 724895 | 1.00E−07 | 1.71 |
| KCNQ2 | 179534 | 1.00E−07 | 1.77 |
| B3GNTL1 | 53341 | 9.00E−07 | 2.04 |
| IMAGE:731616 | 731616 | 1.00E−07 | 2.42 |
| PROZ | 430471 | 1.00E−07 | 2.76 |

TABLE 41

| | OTA- | | |
|---|---|---|---|
| | atypical | Under | |
| name | cloid | p-value | fold |
| LRRC28 | 26519 | 7.00E−04 | 0.43 |
| EPHB6 | 172982 | 3.30E−03 | 0.42 |
| IMAGE:744439 | 744439 | 3.90E−03 | 0.61 |
| ZNF76 | 745003 | 6.40E−03 | 0.61 |
| MGC5395 | 238840 | 8.40E−03 | 0.55 |
| PLXNB2 | 1420676 | 9.10E−03 | 0.53 |
| PTTG1 | 742935 | 9.70E−03 | 0.52 |
| VEGFC | 503189 | 1.24E−02 | 0.64 |
| FOXO1A | 151247 | 1.39E−02 | 0.64 |
| CDH1 | 214008 | 1.44E−02 | 0.56 |
| EMX2 | 365121 | 1.70E−02 | 0.63 |
| SPATA12 | 730300 | 1.93E−02 | 0.66 |
| ASGR1 | 25883 | 1.96E−02 | 0.52 |
| PAX8 | 545475 | 1.98E−02 | 0.50 |
| IMAGE:738332 | 738332 | 2.39E−02 | 0.45 |
| SGSH | 669263 | 3.15E−02 | 0.69 |
| CD63 | 125552 | 3.38E−02 | 0.70 |
| PLA2G6 | 744087 | 3.47E−02 | 0.62 |
| TPM2 | 740620 | 3.48E−02 | 0.58 |
| PBXIP1 | 366042 | 3.56E−02 | 0.63 |
| HMGB2 | 884365 | 3.67E−02 | 0.68 |
| MT1F | 78353 | 3.69E−02 | 0.46 |
| FOXO3A | 109142 | 3.69E−02 | 0.70 |
| LGALS3BP | 742100 | 3.80E−02 | 0.54 |
| MGP | 590264 | 3.87E−02 | 0.50 |
| COL27A1 | 379517 | 3.90E−02 | 0.69 |
| RIPK3 | 667313 | 3.91E−02 | 0.68 |
| TH | 813654 | 4.10E−02 | 0.69 |
| SMARCA5 | 730037 | 4.12E−02 | 0.71 |
| PDLIM1 | 135689 | 4.16E−02 | 0.61 |
| POLR2A | 740130 | 4.38E−02 | 0.69 |
| PTTG1 | 742935 | 7.53E−03 | 0.52 |
| NUMBL | 1855110 | 2.94E−04 | 0.56 |
| NDUFC1 | 796513 | 2.65E−03 | 0.61 |
| USP14 | 376462 | 9.21E−03 | 0.65 |
| PAX6 | 230882 | 1.28E−02 | 0.66 |
| TH | 813654 | 3.81E−02 | 0.69 |
| TCTE3 | 136862 | 2.48E−02 | 0.69 |

TABLE 42

| | OTA- | | |
|---|---|---|---|
| | atypical | Over | |
| name | cloid | p-value | fold |
| LRRIQ2 | 487152 | 4.00E−04 | 2.01 |
| PCGF4 | 740457 | 5.00E−04 | 2.61 |
| IMAGE:667527 | 667527 | 6.00E−04 | 2.45 |
| MAK | 382002 | 1.00E−03 | 2.14 |
| GNAS | 382791 | 1.30E−03 | 2.75 |
| BIN1 | 2384812 | 1.30E−03 | 1.75 |
| COX7B | 566862 | 2.00E−03 | 2.06 |
| TFF1 | 1075949 | 2.20E−03 | 2.61 |
| C21orf84 | 730814 | 2.30E−03 | 2.08 |
| BAK1 | 235938 | 2.40E−03 | 1.80 |
| IMAGE:745072 | 745072 | 2.40E−03 | 2.30 |
| CASP5 | 341763 | 2.60E−03 | 2.72 |
| IMAGE:383718 | 383718 | 2.60E−03 | 1.78 |
| RASAL2 | 486304 | 2.80E−03 | 2.87 |
| IMAGE:379937 | 379937 | 2.90E−03 | 1.98 |
| IMAGE:666279 | 666279 | 3.00E−03 | 2.47 |
| CASP10 | 241481 | 3.50E−03 | 2.21 |
| ARHGAP10 | 731380 | 3.50E−03 | 2.16 |
| BIC | 743270 | 3.60E−03 | 1.78 |
| CD33 | 1917430 | 3.80E−03 | 2.27 |
| IMAGE:682585 | 682585 | 4.40E−03 | 1.66 |
| OAS1 | 666703 | 4.50E−03 | 2.08 |
| CLGN | 1049033 | 5.40E−03 | 1.86 |
| COX5A | 1085884 | 6.10E−03 | 1.70 |
| PARD3 | 724642 | 6.40E−03 | 1.56 |
| DEFB1 | 665086 | 6.60E−03 | 2.79 |
| IMAGE:742061 | 742061 | 7.10E−03 | 1.83 |
| HRK | 767779 | 7.80E−03 | 2.17 |
| IMAGE:729875 | 729875 | 8.10E−03 | 1.68 |
| SCMH1 | 724014 | 8.20E−03 | 2.24 |
| IMAGE:743603 | 743603 | 8.20E−03 | 1.62 |
| TRIM8 | 714498 | 8.70E−03 | 1.61 |
| HMG20A | 731277 | 9.00E−03 | 2.02 |
| IMAGE:727289 | 727289 | 9.20E−03 | 2.17 |
| ULK4 | 744895 | 9.40E−03 | 1.62 |
| JUP | 35628 | 9.90E−03 | 2.28 |
| C6orf176 | 173200 | 1.04E−02 | 1.54 |
| IMAGE:743619 | 743619 | 1.10E−02 | 1.62 |
| MFAP3L | 726821 | 1.11E−02 | 1.53 |
| ABCA8 | 284828 | 1.12E−02 | 1.84 |
| MYC | 129438 | 1.16E−02 | 1.90 |
| IMAGE:731616 | 731616 | 1.17E−02 | 2.28 |
| LOC129531 | 487103 | 1.23E−02 | 1.81 |
| FSHPRH1 | 667355 | 1.31E−02 | 1.57 |
| BAP1 | 1012990 | 1.31E−02 | 1.78 |
| CCDC6 | 487848 | 1.52E−02 | 1.63 |
| FBXO46 | 471664 | 1.58E−02 | 1.78 |
| IMAGE:744899 | 744899 | 1.66E−02 | 1.57 |
| SAFB | 42280 | 1.68E−02 | 1.90 |
| IFITM3 | 713623 | 1.81E−02 | 1.63 |
| SIAT8F | 667110 | 1.96E−02 | 1.81 |
| CCL11 | 502658 | 1.99E−02 | 1.65 |
| OBSCN | 730926 | 2.06E−02 | 1.46 |
| IMAGE:484577 | 484577 | 2.13E−02 | 1.79 |
| PLCG1 | 1174287 | 2.22E−02 | 1.68 |
| ELK4 | 236155 | 2.28E−02 | 1.48 |
| GALNT7 | 381854 | 2.36E−02 | 1.64 |
| KIAA1754L | 365056 | 2.39E−02 | 1.64 |
| JAK1 | 2030501 | 2.58E−02 | 1.60 |
| AURKC | 731021 | 2.60E−02 | 1.67 |
| SLC6A1 | 177967 | 2.73E−02 | 1.44 |
| IMAGE:667258 | 667258 | 2.79E−02 | 1.87 |
| ALDH3A1 | 525221 | 3.08E−02 | 1.65 |
| SIGLEC7 | 743331 | 3.51E−02 | 1.46 |
| LARS | 135407 | 3.76E−02 | 1.41 |
| REV1L | 738916 | 3.80E−02 | 2.08 |
| FLJ20989 | 669557 | 3.92E−02 | 2.37 |
| IMAGE:382521 | 382521 | 4.08E−02 | 1.62 |
| MCC | 731305 | 4.23E−02 | 1.93 |
| SNF7DC2 | 730401 | 1.36E−02 | 1.51 |
| IFITM3 | 713623 | 1.74E−02 | 1.63 |
| BAK1 | 235938 | 1.12E−03 | 1.80 |
| IMAGE:669098 | 669098 | 9.47E−04 | 1.85 |
| CLGN | 1049033 | 1.52E−03 | 1.86 |
| C21orf84 | 730814 | 1.24E−03 | 2.08 |
| MAK | 382002 | 3.71E−04 | 2.14 |
| IMAGE:667527 | 667527 | 2.31E−04 | 2.45 |
| PCGF4 | 740457 | 3.06E−04 | 2.61 |
| GNAS | 382791 | 6.58E−04 | 2.75 |
| DEFB1 | 665086 | 9.17E−04 | 2.79 |

TABLE 43

| | OTC | Under | |
|---|---|---|---|
| name | cloid | p-value | fold |
| MT1F | 78353 | 3.70E−06 | 0.16 |
| IMAGE:359454 | 359454 | 1.31E−05 | 0.27 |
| DCN | 666410 | 6.80E−06 | 0.29 |
| TPM2 | 740620 | 1.59E−05 | 0.30 |
| PREB | 740347 | 1.29E−02 | 0.33 |
| DHRS6 | 364412 | 4.03E−04 | 0.48 |
| DNALI1 | 782688 | 1.00E−07 | 0.24 |
| MT1F | 78353 | 1.00E−07 | 0.16 |
| DCN | 666410 | 1.00E−07 | 0.29 |
| POLR2A | 740130 | 1.00E−07 | 0.43 |
| ZNF76 | 745003 | 1.00E−07 | 0.46 |
| TPM2 | 740620 | 1.00E−04 | 0.30 |
| IMAGE:731726 | 731726 | 1.00E−04 | 0.32 |
| IMAGE:1716286 | 1716286 | 1.00E−04 | 0.30 |
| UBE1L | 250883 | 1.00E−04 | 0.42 |
| PLXNB2 | 1420676 | 1.00E−04 | 0.38 |
| C10orf116 | 740941 | 1.00E−04 | 0.30 |
| PDLIM1 | 135689 | 2.00E−04 | 0.32 |
| PLA2G6 | 744087 | 2.00E−04 | 0.38 |
| C4A | 724366 | 2.00E−04 | 0.24 |
| FOXO3A | 109142 | 3.00E−04 | 0.48 |
| LRP2 | 2055272 | 3.00E−04 | 0.44 |
| GPR124 | 486493 | 4.00E−04 | 0.53 |
| MGP | 590264 | 4.00E−04 | 0.29 |
| MGC5395 | 238840 | 5.00E−04 | 0.44 |
| IFITM3 | 713623 | 7.00E−04 | 0.44 |
| KDR | 469345 | 7.00E−04 | 0.49 |
| MLL | 80688 | 7.00E−04 | 0.47 |
| DJ462O23.2 | 738970 | 7.00E−04 | 0.55 |
| CICE | 725791 | 8.00E−04 | 0.53 |
| DHRS6 | 364412 | 9.00E−04 | 0.48 |
| PBXIP1 | 366042 | 9.00E−04 | 0.44 |
| ETF1 | 146976 | 9.00E−04 | 0.48 |
| TBC1D17 | 739955 | 1.10E−03 | 0.49 |
| CABIN1 | 1844968 | 1.10E−03 | 0.44 |
| GLT8D2 | 365271 | 1.30E−03 | 0.35 |
| TLN1 | 187482 | 1.90E−03 | 0.43 |
| FLT4 | 668815 | 1.90E−03 | 0.53 |
| ZFOC1 | 744606 | 2.10E−03 | 0.50 |
| TIMP3 | 501476 | 2.20E−03 | 0.40 |
| GIMAP5 | 180259 | 2.30E−03 | 0.49 |
| IL11RA | 1101773 | 2.70E−03 | 0.47 |
| E2F5 | 809828 | 2.70E−03 | 0.36 |
| LOC340073 | 731404 | 3.40E−03 | 0.51 |
| PLAC9 | 366115 | 3.60E−03 | 0.44 |
| LOC146909 | 726353 | 3.70E−03 | 0.58 |
| MGC17299 | 713422 | 3.90E−03 | 0.59 |
| CaMKIINalpha | 173820 | 4.10E−03 | 0.53 |
| FLJ22794 | 137454 | 4.10E−03 | 0.55 |
| TTC17 | 665668 | 4.20E−03 | 0.58 |
| LILRA1 | 2032639 | 4.20E−03 | 0.54 |
| APOL3 | 366289 | 4.90E−03 | 0.45 |
| CDH16 | 726763 | 5.00E−03 | 0.40 |
| APLP2 | 549054 | 5.00E−03 | 0.49 |
| NCB5OR | 743367 | 5.70E−03 | 0.43 |
| SEMA7A | 135941 | 6.20E−03 | 0.30 |
| FOLR1 | 131839 | 6.50E−03 | 0.38 |
| IMAGE:46991 | 46991 | 7.40E−03 | 0.48 |
| IMAGE:724416 | 724416 | 7.40E−03 | 0.44 |
| CA11 | 282587 | 7.70E−03 | 0.60 |
| NBL1 | 503874 | 8.50E−03 | 0.32 |
| ZNF395 | 744983 | 8.80E−03 | 0.60 |
| GABRE | 209137 | 9.10E−03 | 0.59 |
| HSPB3 | 502598 | 9.60E−03 | 0.39 |
| ITPR1 | 667348 | 1.00E−02 | 0.58 |
| FLRT3 | 812143 | 1.01E−02 | 0.56 |
| ANKRD20B | 745106 | 1.01E−02 | 0.51 |
| INHBB | 730012 | 1.05E−02 | 0.44 |
| HP1-BP74 | 726092 | 1.11E−02 | 0.53 |
| CRABP1 | 739193 | 1.20E−02 | 0.21 |
| LAPTM4A | 726684 | 1.21E−02 | 0.52 |
| IMAGE:175268 | 175268 | 1.31E−02 | 0.57 |
| PREB | 740347 | 1.34E−02 | 0.33 |
| VCAM1 | 471101 | 1.36E−02 | 0.51 |
| FOXO1A | 151247 | 1.40E−02 | 0.62 |
| EMX2 | 365121 | 1.43E−02 | 0.59 |
| CXCL14 | 345034 | 1.44E−02 | 0.19 |
| FLJ12592 | 252489 | 1.50E−02 | 0.63 |
| PRUNE | 364324 | 1.56E−02 | 0.60 |
| TSHR | 565317 | 1.57E−02 | 0.50 |
| LOC388886 | 34007 | 1.64E−02 | 0.57 |
| TIMP1 | 162246 | 1.72E−02 | 0.35 |
| NR2F1 | 253386 | 1.79E−02 | 0.58 |
| MGAT3 | 731060 | 1.80E−02 | 0.61 |
| IMAGE:738896 | 738896 | 1.87E−02 | 0.26 |
| PET112L | 743125 | 1.91E−02 | 0.60 |
| IMP-2 | 743774 | 1.91E−02 | 0.39 |
| POLI | 137720 | 2.00E−02 | 0.53 |
| FGFRL1 | 739956 | 2.19E−02 | 0.53 |
| SLC25A23 | 741954 | 2.58E−02 | 0.57 |
| PDGFRL | 139242 | 2.70E−02 | 0.56 |
| MGC99813 | 739097 | 2.72E−02 | 0.32 |
| CAV1 | 309645 | 2.72E−02 | 0.52 |
| TNFRSF1B | 73703 | 2.77E−02 | 0.66 |
| IMAGE:342256 | 342256 | 2.82E−02 | 0.30 |
| BAZ2B | 609631 | 2.87E−02 | 0.66 |
| RIPK3 | 667313 | 3.01E−02 | 0.65 |
| ESRRBL1 | 51232 | 3.02E−02 | 0.63 |
| TSC | 745490 | 3.03E−02 | 0.41 |
| AKT1 | 740193 | 3.12E−02 | 0.67 |
| HBA2 | 469647 | 3.13E−02 | 0.31 |
| ZNF251 | 486401 | 3.17E−02 | 0.39 |
| RPS6KA2 | 22711 | 3.20E−02 | 0.57 |
| HA-1 | 484504 | 3.21E−02 | 0.61 |
| VPREB3 | 2349125 | 3.24E−02 | 0.59 |
| PDCD4 | 665376 | 3.29E−02 | 0.58 |
| ISGF3G | 724588 | 3.37E−02 | 0.63 |
| FCGBP | 154172 | 3.38E−02 | 0.42 |
| SPARC | 267358 | 3.51E−02 | 0.66 |
| IMAGE:739953 | 739953 | 3.58E−02 | 0.11 |
| APOD | 838611 | 3.59E−02 | 0.32 |
| CFLAR | 427786 | 3.62E−02 | 0.36 |
| IMAGE:1129745 | 1129745 | 3.64E−02 | 0.11 |
| IMAGE:725758 | 725758 | 3.68E−02 | 0.11 |
| SPATA12 | 730300 | 3.72E−02 | 0.66 |
| IMAGE:1089025 | 1089025 | 3.88E−02 | 0.38 |
| 37500 | 729542 | 3.92E−02 | 0.53 |
| IL19 | 1932168 | 3.94E−02 | 0.58 |
| IMAGE:687667 | 687667 | 4.37E−02 | 0.48 |
| ALDH1A1 | 309697 | 4.38E−02 | 0.53 |
| RODH | 471641 | 4.51E−02 | 0.36 |
| PTPRA | 487130 | 4.55E−02 | 0.46 |
| RFX2 | 731738 | 4.58E−02 | 0.56 |
| PAX8 | 545475 | 4.59E−02 | 0.54 |
| IGLC1 | 160163 | 4.63E−02 | 0.32 |
| HNRPA3 | 365349 | 4.66E−02 | 0.69 |
| APBA3 | 239086 | 4.82E−02 | 0.13 |
| ARHGAP25 | 665982 | 4.92E−02 | 0.57 |
| PSAT1 | 366388 | 4.99E−02 | 0.69 |
| ASGR1 | 25883 | 5.07E−02 | 0.54 |

TABLE 44

| | OTC | Over | |
|---|---|---|---|
| name | cloid | p-value | fold |
| SST | 39593 | 1.34E−03 | 1.81 |
| TH | 813654 | 8.32E−04 | 1.95 |
| TM7SF3 | 666928 | 3.58E−04 | 2.00 |
| KIAA0543 | 175080 | 9.15E−05 | 2.03 |
| ATP11C | 667991 | 2.30E−05 | 2.17 |
| IMAGE:136933 | 136933 | 6.25E−03 | 2.19 |
| IDH3B | 723755 | 1.90E−05 | 2.27 |
| C1QBP | 173371 | 2.70E−06 | 2.47 |
| IMAGE:742061 | 742061 | 1.99E−05 | 2.73 |
| FBXO46 | 471664 | 1.67E−05 | 2.85 |
| CD33 | 1917430 | 1.06E−05 | 3.71 |
| SDHA | 40304 | 1.00E−07 | 3.16 |
| IMAGE:382423 | 382423 | 1.00E−07 | 3.52 |
| C1QBP | 173371 | 1.00E−07 | 2.47 |
| CD33 | 1917430 | 1.00E−07 | 3.71 |
| IMAGE:745072 | 745072 | 1.00E−07 | 3.29 |
| IMAGE:727289 | 727289 | 1.00E−07 | 3.71 |
| VDAC1 | 486221 | 1.00E−07 | 2.67 |
| PCGF4 | 740457 | 1.00E−07 | 3.26 |
| IMAGE:742061 | 742061 | 1.00E−04 | 2.73 |
| COX6A1 | 512910 | 1.00E−04 | 2.29 |
| CCDC6 | 487848 | 1.00E−04 | 2.41 |
| LOC285961 | 742542 | 1.00E−04 | 2.05 |
| RPS6KA5 | 258966 | 1.00E−04 | 2.03 |
| IDH3B | 723755 | 2.00E−04 | 2.27 |
| CCL11 | 502658 | 3.00E−04 | 2.27 |
| FBXO46 | 471664 | 4.00E−04 | 2.85 |
| BAP1 | 1012990 | 4.00E−04 | 2.58 |
| IMAGE:729875 | 729875 | 4.00E−04 | 2.23 |
| DOK5 | 25664 | 5.00E−04 | 2.48 |
| LRRIQ2 | 487152 | 6.00E−04 | 2.24 |
| DCXR | 724596 | 8.00E−04 | 1.85 |
| NAT5 | 731073 | 9.00E−04 | 2.20 |
| TM7SF3 | 666928 | 9.00E−04 | 2.00 |
| FLJ23577 | 668452 | 9.00E−04 | 3.25 |
| RASAL2 | 486304 | 1.00E−03 | 3.40 |
| MAK | 382002 | 1.10E−03 | 2.34 |
| SPI1 | 1285305 | 1.10E−03 | 1.92 |
| BAK1 | 235938 | 1.40E−03 | 2.01 |
| IMAGE:484577 | 484577 | 2.00E−03 | 2.72 |
| CASP5 | 341763 | 2.10E−03 | 3.07 |
| GNAS | 382791 | 2.50E−03 | 2.91 |
| CASP10 | 241481 | 2.70E−03 | 2.54 |
| IMAGE:742837 | 742837 | 2.80E−03 | 2.06 |
| CLECSF12 | 258865 | 2.80E−03 | 4.27 |
| HRK | 767779 | 3.00E−03 | 2.60 |
| COX7B | 566862 | 3.10E−03 | 2.15 |
| CKS2 | 725454 | 3.10E−03 | 2.19 |
| IMAGE:740105 | 740105 | 3.20E−03 | 1.81 |
| AURKC | 731021 | 3.40E−03 | 2.05 |
| TH | 813654 | 3.80E−03 | 1.95 |
| PLCG1 | 1174287 | 4.40E−03 | 2.19 |
| LILRB5 | 71428 | 4.40E−03 | 2.00 |
| CLGN | 1049033 | 4.70E−03 | 2.10 |
| ANP32E | 382738 | 4.80E−03 | 2.28 |
| C20orf18 | 665433 | 5.30E−03 | 2.54 |
| SIAT7B | 823590 | 5.40E−03 | 2.01 |
| DEFB1 | 665086 | 5.50E−03 | 3.53 |
| PDK1 | 1645668 | 6.10E−03 | 1.71 |
| DNMT3B | 645455 | 6.20E−03 | 1.94 |
| SCEL | 668239 | 7.10E−03 | 1.77 |
| GPI | 741474 | 7.50E−03 | 1.80 |
| LOC284669 | 364885 | 7.60E−03 | 1.79 |
| C22orf16 | 236119 | 8.00E−03 | 1.74 |
| JUP | 35628 | 8.10E−03 | 2.61 |
| IMAGE:682585 | 682585 | 8.60E−03 | 1.66 |
| TFF1 | 1075949 | 9.40E−03 | 2.43 |
| SLC9A3R1 | 773286 | 9.90E−03 | 1.94 |
| SIAT8C | 382069 | 1.07E−02 | 1.70 |
| IMAGE:213529 | 213529 | 1.09E−02 | 1.69 |
| IMAGE:668536 | 668536 | 1.11E−02 | 2.60 |
| IMAGE:257555 | 257555 | 1.19E−02 | 1.93 |
| SAFB | 42280 | 1.31E−02 | 2.11 |
| PCSK6 | 31924 | 1.38E−02 | 2.01 |
| ARHGAP10 | 731380 | 1.40E−02 | 2.05 |
| MGC45840 | 725231 | 1.42E−02 | 1.80 |
| CD9 | 727251 | 1.43E−02 | 1.58 |
| MFAP3L | 726821 | 1.50E−02 | 1.55 |
| ELK4 | 236155 | 1.63E−02 | 1.59 |
| DUSP24 | 740158 | 1.78E−02 | 2.84 |
| MEOX1 | 760065 | 1.80E−02 | 1.88 |
| LRPAP1 | 587186 | 1.86E−02 | 1.78 |
| ARVCF | 40633 | 1.97E−02 | 1.56 |
| ALDH1A3 | 486189 | 2.04E−02 | 1.80 |
| C6orf216 | 731742 | 2.19E−02 | 1.79 |
| MLANA | 266361 | 2.20E−02 | 1.54 |
| COX5A | 1085884 | 2.28E−02 | 1.62 |
| GAPD | 152847 | 2.29E−02 | 1.88 |
| IMAGE:669188 | 669188 | 2.30E−02 | 1.84 |
| IMAGE:363955 | 363955 | 2.46E−02 | 1.99 |
| SCMH1 | 724014 | 2.50E−02 | 2.15 |
| TOSO | 813174 | 2.64E−02 | 1.94 |
| ABCA8 | 284828 | 2.66E−02 | 1.78 |
| NOMO1 | 134615 | 2.78E−02 | 1.48 |
| TACC3 | 255398 | 2.80E−02 | 1.60 |
| DPP4 | 343987 | 2.91E−02 | 2.15 |
| SLC36A3 | 731115 | 3.16E−02 | 1.48 |
| C21orf84 | 730814 | 3.31E−02 | 1.71 |
| FLJ25067 | 667252 | 3.44E−02 | 2.19 |
| IFI30 | 740931 | 3.47E−02 | 2.30 |
| IMAGE:743603 | 743603 | 3.53E−02 | 1.53 |
| STX7 | 174396 | 3.81E−02 | 1.89 |
| ZNF319 | 364729 | 3.82E−02 | 2.67 |
| LAMB3 | 1103402 | 3.96E−02 | 1.65 |
| MAD2L2 | 727178 | 4.44E−02 | 1.57 |
| IMAGE:744899 | 744899 | 4.52E−02 | 1.50 |
| NTRK2 | 2048801 | 4.54E−02 | 1.95 |
| IMAGE:382521 | 382521 | 4.94E−02 | 1.67 |
| BST2 | 811024 | 4.94E−02 | 1.49 |

TABLE 45

| | PTC | Under | |
|---|---|---|---|
| name | cloid | p-value | fold |
| MATN1 | 1624260 | 1.00E−07 | 0.42 |
| MATN1 | 1624260 | 1.00E−07 | 0.42 |
| IMAGE:743517 | 743517 | 1.00E−07 | 0.57 |
| MATN2 | 28584 | 1.00E−07 | 0.39 |
| DIO1 | 296702 | 1.00E−07 | 0.36 |
| VAV1 | 80384 | 1.00E−07 | 0.35 |
| FLJ20989 | 669557 | 1.00E−07 | 0.28 |
| MGC20781 | 731227 | 1.00E−07 | 0.54 |
| FKBP6 | 795736 | 1.00E−07 | 0.51 |
| SLC4A4 | 787938 | 1.00E−07 | 0.51 |
| IMAGE:384844 | 384844 | 1.00E−07 | 0.55 |
| FLJ11184 | 384404 | 1.00E−07 | 0.48 |
| HSPB3 | 502598 | 1.00E−07 | 0.44 |
| RORA | 1086823 | 1.00E−07 | 0.60 |
| DAB1 | 382621 | 1.00E−07 | 0.63 |
| IMAGE:1716286 | 1716286 | 1.00E−07 | 0.48 |
| SIAT7B | 823590 | 1.00E−07 | 0.61 |
| RNF32 | 731422 | 1.00E−07 | 0.49 |
| IGHMBP2 | 23876 | 1.00E−04 | 0.55 |
| ASGR1 | 25883 | 1.00E−04 | 0.51 |
| IMAGE:667258 | 667258 | 1.00E−04 | 0.51 |
| ZNF319 | 364729 | 2.00E−04 | 0.35 |
| F5 | 433155 | 2.00E−04 | 0.60 |
| DEPDC6 | 669318 | 3.00E−04 | 0.60 |
| REV1L | 738916 | 7.00E−04 | 0.47 |
| IMAGE:727289 | 727289 | 7.00E−04 | 0.56 |
| MGC23909 | 731598 | 1.00E−03 | 0.53 |
| MGC99813 | 739097 | 1.00E−03 | 0.40 |
| IMAGE:383718 | 383718 | 1.20E−03 | 0.65 |
| PPIL4 | 364777 | 1.30E−03 | 0.64 |
| IMAGE:667527 | 667527 | 1.30E−03 | 0.61 |
| C20orf19 | 366032 | 1.40E−03 | 0.71 |
| IMAGE:744074 | 744074 | 1.50E−03 | 0.46 |
| IMAGE:666315 | 666315 | 1.50E−03 | 0.65 |
| DCXR | 724596 | 1.50E−03 | 0.73 |
| SCMH1 | 724014 | 1.60E−03 | 0.56 |
| PTTG1 | 742935 | 1.80E−03 | 0.61 |
| KLK2 | 1102600 | 1.80E−03 | 0.70 |
| NME7 | 743982 | 1.90E−03 | 0.48 |
| IMAGE:744616 | 744616 | 2.00E−03 | 0.71 |
| PARD3 | 724642 | 2.00E−03 | 0.73 |
| CDH16 | 726763 | 2.10E−03 | 0.55 |
| SYP | 30471 | 2.20E−03 | 0.59 |
| IMAGE:726513 | 726513 | 2.30E−03 | 0.74 |
| BIN1 | 2384812 | 2.30E−03 | 0.72 |
| ALDH1A1 | 309697 | 2.60E−03 | 0.57 |
| PCGF4 | 740457 | 2.60E−03 | 0.60 |
| OBSCN | 730926 | 2.60E−03 | 0.73 |
| USP13 | 666007 | 2.80E−03 | 0.67 |
| SIAT8C | 382069 | 2.80E−03 | 0.70 |
| SEMA7A | 135941 | 2.90E−03 | 0.47 |
| IMAGE:742061 | 742061 | 3.10E−03 | 0.66 |
| IMAGE:731616 | 731616 | 3.50E−03 | 0.56 |
| MT1F | 78353 | 3.70E−03 | 0.49 |
| TOSO | 813174 | 3.70E−03 | 0.62 |
| IMAGE:485104 | 485104 | 3.90E−03 | 0.60 |
| IMAGE:2484270 | 2484270 | 4.20E−03 | 0.67 |
| IMAGE:744439 | 744439 | 4.40E−03 | 0.74 |
| ME3 | 724238 | 4.70E−03 | 0.61 |
| DUSP24 | 740158 | 4.90E−03 | 0.49 |
| GNAS | 382791 | 5.70E−03 | 0.58 |
| KLF1 | 208991 | 5.90E−03 | 0.60 |
| MCC | 731305 | 5.90E−03 | 0.57 |
| JUP | 35628 | 5.90E−03 | 0.59 |
| KCNQ2 | 179534 | 6.60E−03 | 0.68 |
| AURKC | 731021 | 6.90E−03 | 0.68 |
| PRUNE | 364324 | 7.10E−03 | 0.73 |
| MFAP3L | 726821 | 7.10E−03 | 0.76 |
| OAS1 | 666703 | 7.60E−03 | 0.65 |
| TTC17 | 665668 | 8.60E−03 | 0.76 |
| HSPC159 | 365045 | 9.60E−03 | 0.72 |
| FLJ23577 | 668452 | 9.60E−03 | 0.60 |
| E2F5 | 809828 | 1.00E−02 | 0.60 |
| ANP32E | 382738 | 1.03E−02 | 0.66 |
| PLCG2 | 201467 | 1.05E−02 | 0.70 |
| IMAGE:363955 | 363955 | 1.09E−02 | 0.64 |
| C21orf84 | 730814 | 1.11E−02 | 0.69 |
| CRABP1 | 739193 | 1.12E−02 | 0.41 |
| HMG20A | 731277 | 1.13E−02 | 0.66 |
| SH3RF2 | 744797 | 1.14E−02 | 0.70 |
| PRKACB | 366820 | 1.14E−02 | 0.73 |
| MAPRE2 | 383868 | 1.16E−02 | 0.61 |
| IMAGE:682585 | 682585 | 1.20E−02 | 0.75 |
| MAK | 382002 | 1.22E−02 | 0.71 |
| IMAGE:745072 | 745072 | 1.23E−02 | 0.67 |
| BIC | 743270 | 1.25E−02 | 0.73 |
| IMAGE:742837 | 742837 | 1.31E−02 | 0.74 |
| SDHA | 40304 | 1.47E−02 | 0.75 |
| CD33 | 1917430 | 1.56E−02 | 0.66 |
| GNB4 | 813260 | 1.57E−02 | 0.67 |
| IMAGE:744055 | 744055 | 1.59E−02 | 0.63 |
| IMAGE:379937 | 379937 | 1.62E−02 | 0.71 |
| IMAGE:136686 | 136686 | 1.66E−02 | 0.73 |
| BAP1 | 1012990 | 1.69E−02 | 0.70 |
| SPI1 | 1285305 | 1.70E−02 | 0.77 |
| RASAL2 | 486304 | 1.71E−02 | 0.60 |
| C20orf18 | 665433 | 1.73E−02 | 0.65 |
| LOC285961 | 742542 | 1.79E−02 | 0.78 |
| MUTED | 731202 | 1.80E−02 | 0.68 |
| TFF1 | 1075949 | 1.82E−02 | 0.63 |
| FLT4 | 668815 | 1.87E−02 | 0.77 |
| ULK4 | 744895 | 2.10E−02 | 0.76 |
| NTRK2 | 2048801 | 2.14E−02 | 0.65 |
| IMAGE:744385 | 744385 | 2.40E−02 | 0.73 |
| SAFB | 42280 | 2.58E−02 | 0.70 |
| SIAT8F | 667110 | 2.87E−02 | 0.70 |
| PCSK6 | 31924 | 2.92E−02 | 0.72 |
| ABCA8 | 284828 | 3.07E−02 | 0.73 |
| APIN | 364706 | 3.11E−02 | 0.74 |
| SLC6A1 | 177967 | 3.20E−02 | 0.80 |
| LILRB5 | 71428 | 3.23E−02 | 0.74 |
| OSR1 | 364686 | 3.50E−02 | 0.77 |
| DKFZp761P0423 | 730829 | 3.54E−02 | 0.71 |
| HBA2 | 469647 | 3.59E−02 | 0.52 |
| GNAI1 | 753215 | 3.65E−02 | 0.77 |
| SIAT8B | 33133 | 3.96E−02 | 0.81 |
| PLEK | 724225 | 4.01E−02 | 0.73 |
| KIAA1196 | 738938 | 4.06E−02 | 0.79 |
| PROZ | 430471 | 4.15E−02 | 0.60 |
| BAK1 | 235938 | 4.15E−02 | 0.79 |
| HRK | 767779 | 4.45E−02 | 0.69 |
| ARHGAP10 | 731380 | 4.80E−02 | 0.72 |
| CBL | 1578721 | 5.10E−02 | 0.75 |
| ZBTB16 | 2467442 | 5.35E−02 | 0.69 |

TABLE 46

| | PTC | Over | |
|---|---|---|---|
| name | cloid | p-value | fold |
| CD9 | 727251 | 1.45E−05 | 1.57 |
| DAF | 627107 | 1.00E−07 | 1.89 |
| ADAMTS9 | 376153 | 6.40E−06 | 1.95 |
| PET112L | 743125 | 1.00E−07 | 1.96 |
| GSS | 140405 | 1.00E−07 | 2.05 |
| CASP3 | 823680 | 6.00E−07 | 2.08 |
| IMAGE:136976 | 136976 | 1.00E−07 | 2.11 |
| CCND1 | 324079 | 1.00E−07 | 2.55 |
| SLPI | 378813 | 1.00E−07 | 2.98 |
| IMAGE:687667 | 687667 | 1.00E−07 | 3.74 |
| CITED1 | 265558 | 1.00E−07 | 4.88 |
| CDH3 | 359051 | 1.00E−07 | 3.95 |
| DPP4 | 343987 | 1.00E−07 | 4.22 |
| ECM1 | 301122 | 1.00E−07 | 6.00 |
| CLDN1 | 594279 | 1.00E−07 | 8.98 |
| TSC | 745490 | 1.00E−07 | 4.65 |
| QPCT | 711918 | 1.00E−07 | 3.36 |
| IMAGE:687667 | 687667 | 1.00E−07 | 3.74 |
| SLPI | 378813 | 1.00E−07 | 2.98 |
| CITED1 | 265558 | 1.00E−07 | 4.88 |
| TIMP1 | 162246 | 1.00E−07 | 4.45 |
| KISS1 | 812955 | 1.00E−07 | 1.96 |
| TARP | 145105 | 1.00E−07 | 2.55 |
| CAPN3 | 757248 | 1.00E−07 | 1.86 |
| CCND1 | 324079 | 1.00E−07 | 2.55 |
| LOC284739 | 741587 | 1.00E−07 | 2.64 |
| PREB | 740347 | 1.00E−07 | 4.28 |
| RWDD1 | 33470 | 1.00E−07 | 2.17 |
| ABCC3 | 208097 | 1.00E−07 | 3.48 |
| PET112L | 743125 | 1.00E−07 | 1.96 |
| FMO5 | 364526 | 1.00E−07 | 2.04 |
| IMAGE:136976 | 136976 | 1.00E−07 | 2.11 |
| POU2F2 | 188393 | 1.00E−07 | 2.25 |
| NOTCH2 | 1641901 | 1.00E−07 | 2.59 |
| GSS | 140405 | 1.00E−07 | 2.05 |
| DAF | 627107 | 1.00E−07 | 1.89 |
| INHBB | 730012 | 1.00E−07 | 2.51 |
| FOXQ1 | 725511 | 1.00E−07 | 2.26 |
| NOTCH1 | 359461 | 1.00E−07 | 1.80 |
| IMAGE:726106 | 726106 | 1.00E−07 | 1.87 |
| IMAGE:744881 | 744881 | 1.00E−07 | 1.97 |
| COL8A2 | 486204 | 1.00E−07 | 1.86 |
| SCEL | 668239 | 1.00E−07 | 1.78 |
| CASP3 | 823680 | 1.00E−07 | 2.08 |
| RASGRP1 | 725707 | 1.00E−07 | 2.64 |
| CD5 | 356841 | 1.00E−07 | 1.66 |
| IMAGE:251427 | 251427 | 1.00E−07 | 1.82 |
| TBC1D2 | 236331 | 1.00E−07 | 1.80 |
| IMAGE:1877668 | 1877668 | 1.00E−07 | 1.70 |
| KCNK5 | 134978 | 1.00E−07 | 1.91 |
| MDK | 309009 | 1.00E−07 | 2.78 |
| ADAMTS9 | 376153 | 1.00E−07 | 1.95 |
| IMAGE:234148 | 234148 | 1.00E−07 | 1.89 |
| FLJ12604 | 731128 | 1.00E−07 | 1.83 |
| LEMD1 | 731047 | 1.00E−07 | 1.97 |
| CD9 | 727251 | 1.00E−07 | 1.57 |
| HDAC1 | 1896337 | 1.00E−07 | 1.70 |
| NRIP1 | 38775 | 1.00E−07 | 1.60 |
| NCB5OR | 743367 | 1.00E−07 | 2.00 |
| STAT2 | 2306096 | 1.00E−07 | 1.54 |
| TIAM1 | 23612 | 1.00E−07 | 1.85 |
| CD63 | 125552 | 1.00E−07 | 1.52 |
| IMP-2 | 743774 | 1.00E−07 | 2.58 |
| SPINL | 236399 | 1.00E−07 | 2.55 |
| IMAGE:2266583 | 2266583 | 1.00E−07 | 1.60 |
| LOC126669 | 682088 | 1.00E−07 | 1.65 |
| TRAF2 | 966894 | 1.00E−07 | 1.58 |
| S100A1 | 175772 | 1.00E−07 | 1.90 |
| CA11 | 282587 | 1.00E−07 | 1.49 |
| DJ462O23.2 | 738970 | 1.00E−04 | 1.55 |
| ARMCX3 | 251452 | 1.00E−04 | 1.71 |
| IMAGE:738332 | 738332 | 1.00E−04 | 2.51 |
| FLRT3 | 812143 | 1.00E−04 | 1.64 |
| LAMB3 | 1103402 | 1.00E−04 | 1.72 |
| IMAGE:2113771 | 2113771 | 1.00E−04 | 1.53 |
| S100A4 | 868577 | 2.00E−04 | 2.72 |
| RFX2 | 731738 | 2.00E−04 | 1.91 |
| APLP2 | 549054 | 2.00E−04 | 1.80 |
| TM4SF8 | 713647 | 2.00E−04 | 1.52 |
| C19orf33 | 379540 | 2.00E−04 | 1.74 |
| MRC2 | 235882 | 3.00E−04 | 1.50 |
| IL19 | 1932168 | 6.00E−04 | 1.71 |
| KRT18 | 725096 | 7.00E−04 | 1.52 |
| ECRG4 | 472114 | 7.00E−04 | 1.95 |
| MGAT3 | 731060 | 7.00E−04 | 1.48 |
| BID | 128065 | 8.00E−04 | 1.49 |
| TLX3 | 1860115 | 9.00E−04 | 1.74 |
| DUSP5 | 33285 | 1.00E−03 | 2.15 |
| PDLIM1 | 135689 | 1.50E−03 | 1.65 |
| MLANA | 266361 | 1.50E−03 | 1.41 |
| FLJ22794 | 137454 | 1.90E−03 | 1.45 |
| SPATA12 | 730300 | 2.00E−03 | 1.43 |
| IMAGE:136014 | 136014 | 2.00E−03 | 1.55 |
| LGALS3BP | 742100 | 2.20E−03 | 1.78 |
| IMAGE:731751 | 731751 | 2.20E−03 | 1.75 |
| NFIB | 416959 | 2.30E−03 | 1.42 |
| GABRE | 209137 | 2.50E−03 | 1.43 |
| RPS6KA2 | 22711 | 3.00E−03 | 1.55 |
| S100A13 | 1104732 | 3.10E−03 | 1.44 |
| CLDN7 | 300268 | 3.60E−03 | 1.35 |
| FRMD4B | 669564 | 3.70E−03 | 1.55 |
| SMARCA5 | 730037 | 4.60E−03 | 1.37 |
| SLC6A8 | 725877 | 5.20E−03 | 2.57 |
| RBBP4 | 705147 | 5.30E−03 | 1.33 |
| FLJ35794 | 364204 | 5.40E−03 | 1.52 |
| PSAT1 | 366388 | 5.60E−03 | 1.36 |
| FLJ10748 | 726830 | 6.40E−03 | 1.38 |
| S100A10 | 119939 | 6.50E−03 | 1.60 |
| STAT1 | 110101 | 6.80E−03 | 1.32 |
| GFM2 | 364959 | 9.70E−03 | 1.33 |
| HCA112 | 486201 | 9.80E−03 | 1.53 |
| NR2F1 | 253386 | 1.06E−02 | 1.39 |
| PDCD4 | 665376 | 1.12E−02 | 1.43 |
| DHRS6 | 364412 | 1.19E−02 | 1.35 |
| KLF4 | 188232 | 1.38E−02 | 1.56 |
| CKS2 | 725454 | 1.78E−02 | 1.46 |
| HP1-BP74 | 726092 | 1.81E−02 | 1.36 |
| URB | 809719 | 2.56E−02 | 1.46 |
| IMAGE:741835 | 741835 | 2.77E−02 | 1.30 |
| DSP | 135975 | 3.41E−02 | 1.30 |
| CXCL14 | 345034 | 3.42E−02 | 2.26 |
| ITGB7 | 1337232 | 3.64E−02 | 1.25 |
| C4A | 724366 | 3.90E−02 | 1.64 |
| IMAGE:136801 | 136801 | 4.25E−02 | 1.23 |
| IQCA | 669510 | 4.46E−02 | 1.24 |
| PBXIP1 | 366042 | 4.55E−02 | 1.32 |
| LOC286170 | 134858 | 4.70E−02 | 1.31 |

1. An integrated in vitro method for the prediction or for the diagnosis of a specific thyroid disease in a patient comprising the steps of:
a) providing a thyroid tissue sample previously collected from the said patient;
b) quantifying, in the said thyroid tissue sample, the expression level of two or more disease-specific marker genes that are indicative of the risk of occurrence of, or of the occurrence of, a specific thyroid disease, wherein the said two or more disease-specific marker genes are selected from two or more of the groups of marker genes consisting of:
(i-1) 669510, 725096, 366100, 325155, 154172, 787938, 744055, 592276, 740941, 73778, 667174, 739193, 295412, 23827, 136686, 744385, 471641, 724276, 28584, 268972, 739097, 744943, 724609, 2074228, 365913, 687782, 23872, 668452, 681959, 687379, 23271, 731745, 738332, 726760, 666911, 724150, 682555, 587186, 813260, 136646, 309645, 809828, 487152, 564994, 667348, 730149, 731115, 738945, 985457, 179288, 742862, 78353, 503189, 1087348, 753215, 471574, 471696, 549054, 744657, 731357, 713422, 486708, 666794, 726830, 730829, 300268, 725364, 665376, 375746, 324079, 365271, 172982, 724562, 839101, 265558, 52226, 713922, 136919, 682119, 745106, 1877668, 1594414, 471829, 739956, 23945, 173841, 31120, 342181, 742580, 135941, 32339, 687912, 666154, 743774, 738970, 22711, 501971, 364846, 175864, 666792, 724416, 174683, 667892, 744087, 68950, 2467442, 136807, 744439, 1716286, 121551, 744797, 742061, 666778, 740158, 724596, 143759, 666315, 50202, 667514, 364959, 258666, 148796, 364324, 72744, 669263, 667533, 365177, 2055272, 731298, 512910, 470408, 137720, 2237263, 712295, 284828, 81911, 251452, 135705, 153667, 666007, 2266583, 136863, 724119, 136223, 727251, 485872, 137238, 713653, 364412, 471702, 668684, 727056, 665674, 251250, 364706, 136260, 136560, 738938, 382423, 430263, 2139164, 309697, 773192, 726272, 682088, 666367, 21716, 135689, 383945, 727289, 724554, 364729, 665649, 668152, 811582, 666551, 472111, 489945, 486304, 416744, 258865, 744050, 743016, 1846326, 509606, 666110, 51232, 3659591, 667355, 364974, 666860, 179266, 727092, 24176, 725076, 137353, 307094, 23765, 666168, 725836, 172996, 364926, 525221, 472160, 667110, 730012, 135980, 136014, 2046361, 726763, 731726, 135887, 365288, 44881, 724508, 146976, 744616, 366159, 724895, 724892, 668912, 743987, 23817, 665538, 687625, 136606, 713879, 26164, 667587, 731410, 743422, 687532, 726335, 669318, 214611, 669181, 713647, 178792, 1417901, 135766, 364469, 565317, 382738, 669188, 666361, 667117, 591907, 173288, 257555, 214008, 730938, 868169, 731404, 667112, 135773, 34007, 1521706, 687679, 730534, 33621, 664233, 135125, 155345, 208991, 177827, 667353, 745003, 731685, 79729, 25664, 139242, 666140, 270549, 173200, 364777, 740381, 795309, 731423, 49888, 741891, 232714, 743290, 133531, 665379, 669319, 258127, 1238492, 682555, 726821, 1420676, 364510, 668186, 44193, 365045, 32917, 1174287, 725371, 119772, 730834, 252382, 179214, 1288183, 628418, 382791, 2384812, 364822, 726513, 282587, 731047, 25883, 134858, 207920, 668476, 251147, 667259, 38403, 365311, 178908, 1911930, 135379, 681875, 149809, 971276, 687667, 738558, 136952, 687287, 744606, 743118, 364352, 667375, 726647, 134978, 726675, 743394, 738424, 729929, 782688, 726782, 135303, 742695, 363955, 731348, 366889, 194350, 436554, 668536, 725340, 174861, 1859532, 261500, 714414, 667361, 742739, 364741, 366032, 729510, 745490, 731021, 342551, 740457, 726596, 2032639, 136121, 745072, 665086, 738966, 731073, 135407, 471664, 301122, 738506, 136747, 714437, 485104, 179733, 713913, 742542, 743760, 26651, 743519, 196992, 665392, 364717, 731277, 667067, 136868, 564878, 743245, 668089, 136399, 383718, 741954, 742590, 544683, 134976, 365589, 730300, 381854, 731305, 2348237, 669564, 714049, 366067, 487148, 668239, 1837488, 359202, 266361, 172785, 25895, 545475, 501527, 726981, 359051, 230126, 724642, 668815, 687852, 26455, 286138, 682507, 135539, 469549, 687953, 366085, 744395, 726353, 324666, 135352, 25865, 175772, 134671, 730699, 22908, 343987, 366842, 323438, 26519, 366042, 727067, 365121, 359461, 1104732, 135630, 135975, 238705, 472114, 502598, 645455, 669574, and 705147,
(i-2) 43541, 687990, 365990, 562904, 725548, 365877, 742763, 180259, 2449149, 1089025, 713623, 382760, 258747, 743961, 174396, 724306, 1505308, 726454, 665452, 665833, 1352408, 731060, 73703, 207926, 489326, 1743279, 139073, 1626951, 1578721, 188393, 156720, 781738, 1645668, 714493, 1896337, 195340, 203166, 310519, 503874, 743722, 324066, 738944, 201467, 1870594, 122874, 140405, 204686, 2497617, 2306096, 1707527, 609631, 356960, 2345206, 740931, 342256, 725473, 682207, 2242054, 868577, 727154, 279470, 813174, 252489, 2341829, 729924, 263251, 250883, 738896, 1326169, 448163, 731648, 2113771, 365349, 731023, 744980, 668584, 727496, 132933, 592598, 46991, 128065, 730037, 356841, 2238108, 823680, 292042, 504539, 485803, 340657, 590264, 1084386, 742853, 210587, 884365, 134615, 740927, 1088781, 2091591, 667239, 727430, 486401, 1286238, 742577, 1916307, 3054031, 366289, 366233, 80688, 665632, 379200, 162246, 743246, 486935, 287843, 365755, 811024, 489249, 840753, 731689, 50491, 1422194, 1251197, 1942634, 725268, 666410, 743415, 364448, 743367, 1287638, 668442, 28511, 1422791, 743224, 124701, 725612, 471568, 156183, 840687, 731751, 1705397, 26061, 666061, 725345, 740347, 346610, 131839, 123950, 2336358, 135801, 666425, 809959, 1550616, 2148946, 2728204, 366585, 667727, 116906, 288807, 345034, 768241, 667440, 1089513, 1131054, 1256485, 245426, 427786, 742696, 744911, 1013595, 1056782, 1057773, 1072914, 1086914, 110101, 1101773, 110938, 111460, 1116948, 1129745, 114008, 1146624, 117072, 1188588, 120649, 120916, 122063, 124554, 127360, 1285305, 129438, 1324060, 1337232, 134034, 137238, 137454, 1420842, 143957, 149910, 152398, 152802, 152844, 153411, 1541958, 1544175, 155345, 155717, 1578721, 1580147, 159381, 160163, 1632468, 1641901, 1654256, 1712870, 1735081, 1737165, 177967, 178984, 179857, 180950, 1844689, 187482, 1899942, 191881, 1929371, 1932202, 195712, 196115, 1982430, 200018, 202897, 204381, 209137, 2091389, 2103105, 2108870, 2137932, 21496, 2158322, 23121, 232002, 232507, 234357, 2349125, 235882, 235934, 236155, 2361911, 236386, 2364998, 2366175, 239086, 2398980, 239951, 243741, 248811, 251047, 255398, 259552, 271985, 276658, 276727, 285666, 285693, 289337, 292882, 295093, 3058557, 308564, 324140, 324225, 325008, 33285, 342416, 343867, 34783, 35356, 36371, 364204, 364271, 366359, 377560, 379540, 382612, 38775, 40633, 43629, 44064, 471101, 471210, 471947, 484504, 485085, 485725, 486201, 486204, 486779, 487103, 487130, 487436, 490710, 49725, 502658, 52740, 566585, 5725785, 665566, 665651, 665784, 665881, 665982, 666510, 666755, 667144, 667435, 667602, 668454, 668494, 669503, 713896, 714216, 723753, 723938, 724042, 724225, 724366, 724588, 725622, 725696, 725758, 725927, 726147, 726156, 726424, 726557, 726904, 727173, 727207, 727394, 727521, 727554, 729489, 730032, 730271, 730410, 730413, 730865, 730968, 730971, 731007, 731098, 731152, 731284, 731762, 738315, 739953, 740105, 740193, 740604, 740617, 740885, 740971, 741389, 741661, 741835, 741919, 741988, 742555, 742574, 742592, 742649, 742839, 742899, 742965, 742977, 743113, 743212, 743237, 743259, 743426, 744366, 744413, 744468, 744755, 744756, 744881, 744897, 744926, 745018, 745125, 745223, 745248, 745326, 773286, 782730, 78294, 80109, 812143, 824531, 825265, 838611, 851264, 924826, 950695, and 964736.
(ii) marker genes specific for Grave's disease (GD that are contained as inserted DNA in the recombinant vectors selected from the group of vectors having the following IMAGE clone ID No:
(ii-1) 25664, 868169, 731380, 1049033, 730942, 731616, 726578, 366388, 485872, 1662274, 2074228, 730926, 745106, 731021, 207920, 258865, 503189, 745490, 1103402, 1911930, 731305, 173296, 669181, 727491, 713879, 359461, 265558, 687667, 668239, 232366, 730814, 484535, 743774, 667587, 665086, 366889, 723950, 256177, 1859532, 665433, 985457, 564878, 376356, 383868, 471664, 724642, 23817, 724596, 487152, 669549, 155345, 347362, 666425, 24541, 290265, 667252, 725231, 343987, 705147, 1238492, 745072, 544683, 3054031, 744604, 731073, 2237263, 136919, 486-447, 740620, 1420676, 151247, 738558, 731645, 179288, 667683, 135125, 723914, 666110, 381854, 365056, 738970, 724554, 301122, 382521, 341763, 136747, 364717, 484577, 668510, 145696, 594279, 725340, 712118, 82687, 252291, 666551, 713859, 359051, 786662, 741497, 731298, 729957, 743903, 682555, 30981, 666778, 135407, 742919, 378458, 743579, 258127, 365613, 357807, 383528, 486189, 738938, 279172, 665649, 172828, 738332, 549054, 742695, 213529, 667048, 307094, 436554, 324079, 365526, 471574, 742580, 666277, 2348237, 485652, 744606, 52226, 236119, 730924, 173200, 342551, 744087, 345034, 731590, 743118, 261500, 136223, 668536, 666928, 730149, 180851, and 24623
(ii-2) 687990, 743961, 486493, 379279 and 667313, 726424, 122063, 729489, 724588, 343867, 1116948, 727154, 1632468, 1057773, 117072, 73703, 590264, 726147, 1089513, 724225, 80109, 744385, 1654256, 964736, 667435, 365349, 742696, 295093, 110938, 195712, 245426, 127360, 122874, 448163, 489326, 501476, 202897, 23121, 744911, 743212, 49725, 725548, 191881, 744926, 665452, 2158322, 727394, 811024, 259552, 741389, 232507, 342416, 2238108, 324225, 366359, 1188588, 742899, 823680, 782730, 204381, 727207, 243741, 120649, 366115, 740885, 726763, 258747, 727554, 153411, 665566, 1286238, 135773, 136686, 731711, 250771, 122063, 744385, 740140, 262894, 53081, 258698, 53119, 136366, 665674, 178161, and 729489,
(iii) marker genes specific for macrofollicular adenoma (FTA-a) that are contained as inserted DNA in the recombinant vectors selected from the group of vectors having the following IMAGE clone ID No:
(iii-1) 2348237, 135379, 743851, 1712992, 376699, 1251197, 1174287, 173841, 486221, 265345, 666564, 252382, 173371, 731073, 253386, 1285305, 342700, 179214, 3054031, 359797, 486189, 258865, 731742, 666061, 135705, 745072, 731044, 731616, 2148946, 258966, 504539, 725188, 213529, 742919, 682585, 666554, 825224, 1049033, 365045, 742837, 127507, 725198, 116906, 135875, 135773, 486282, 343987, 232366, 80162, 566862, 724544, 731115, 725454, 813174, 340657, 838611, 730534, 742763, 178255, 687336, 742542, 667683, 725473, 139073, 731202, 594279, 727289, 382423, 724556, 740457, 682276, 666361, 713879, 1016390, 667110, 359461, 25664, 743519, 486540, 1645668, 744899, 562904, 1174342, 258127, 1916307, 665693, 714109, 665649, 687270, 1942634, 1352408, 364686, 357442, 729957, 667598, 739247, 382738, 145696, 1578721, 544683, 745512, 249311, 705147, 727482, 365526, 724884, 1575056, 739432, 743224, 730779, 137238, 284828, 667048, 739237, 731047, 1326169, 364570, 174396, 665086, 235938, 666860, 743394, 758037, 740090, 594655, 760065, 731076, 1188588, 137478, 868169, 1012990, 382069, 666425, 135203, 341763, 669549, 726894, 731277, 485104, 668239, 682207, 741406, 201467, 1088781, 382002, 236155, 731758, 364934, 235882, 178792, 365056, 668152, 665452, 136646, 666110, 668146, 347503, 744616, 177857, 258747, 665156, 1308954, 471664, 485872, 587186, 136919, 504596, 265558, 135407, 156183, 484577, 725707, 173200, 137454, 743078, 155345, 666794, 667375, 135303, 134976, 301122, 741497, 725585, 743579, 263251, 744074, 42280, 742695, 731598, 666426, 743603, 665403, 188232, 1859532, 290265, 884365, 512910, 687953, 731357, 723950, 136747, 31924, 279470, 741790, 781738, 204686, 666334, 119939, 1085884, 489326, 356841, 743982, 1707527, 35628, 730814, 738358, 667252, 740927, 342581, 137131, 2150502, 2032639, 2306096, 767779, 743182, 309697, 744374, 2238108, 136223, 666279, 726821, 383945, 726618, 23817, 188393, 741474, 25895, 135980, 136014, 741066, 382521, 731275, 136598, 2345206, 382791, 724238, 485803, 725970, 682119, 276727, 668476, 713647, 742061, 1075949, 241481, 347362, 725548, 1492440, 1117183, 726600, 489249, 2345206, 327182, 133531, 2214396, 71428, 665342, 742743, 666235, 743259, 2030501, 666703, 179534, 744983, 356960, 1561035, 726647, 1741589, 137719, 2048801, 487148, 33664, 43541, 744895, 687667, 132752, 122874 and 2032639, 1116948, 129438, 195712, 1982430, 2103105, 21496, 2384812, 24065, 364271, 364717, 40633, 485085, 487379, 723755, 729875, and 753215,
(iii-2) 745003, 1844968, 364324, 357201, 24392, 1420676, 687532, 724562, 729510, 725371, 24083, 738938, 730938, 148796, 137205, 174683, 21808, 3659591, 325155, 666792, 154172, 295412, 176543, 24532, 359202, 68950, 2046361, 725364, 741178, 668360, 300194, 23872, 364352, 364469, 726353, 742100, 23271, 667112, 25949, 22711, 744055, 430263, 738506, 738945, 743245, 812967, 744087, 250883, 741954, 667892, 364974, 744505, 187482, 52226, 23945, 179288, 738507, 1526058, 782688, 174861, 669295, 713859, 136868, 24300, 731726, 682251, 21716, 38789, 25883, 172693, 725340, 669141, 712295, 23641, 738944, 1716286, 723867, 726272, 23827, 357807, 136801, 135887, 2254555, 53333, 472111, 214008, 744106, 136462, 725533, 366042, 173820, 136114, 364575, 68972, 687679, 366436, 2237263, 32339, 26486, 1698236, 279150, 1101773, 666986, 669470, 257259, 667313, 128694, 178750, 2341829, 472160, 727123, 1087348, 135689, 135941, 151247, 172828, 172982, 208991, 33621, 376003, 502598, 545475, 549054, 666007, 667174, 667348, 724366, 724416, 729971, 731404, 739955, 740130, 740941, 742580, 743118, 744385, 744606, 744797, 745106, 745465, 78353, and 809828,
(iv) marker genes specific for atypical follicular adenoma (FTA-aty) that are contained as inserted DNA in the recombinant vectors selected from the group of vectors having the following IMAGE clone ID No:
(iv-1) 503874, 666128, 838611, 208991, 739097, 1089025, 725548, 739193, 155839, 28584, 202897, 743331, 743619, 725345, 345034, 235938, 729924, 681957, 30219, 773330, 665632, 2048801, 760065, 119939, 50491, 195340, 740941, 201467, 1581686, 743603, 207920, 731616, 71428, 30471, 504646, 487848, 342581, 1942634, 2238108, 740158, 42280, 196338, 742739, 743615, 1074708, 366484, 30981, 743246, 592598, 485104, 174396, 364777, 379540, 3054031, 123950, 1536968, 366100, 489326, 24587, 665086, 342256, 448163, 35628, 544818, 179534, 665156, 309645, 726454, 738896, 31924, 665452, 564878, 1286238, 379517, 177857, 744410, 730926, 809828, 288807, 740105, 366842, 146049, 713831, 140347, 666279, 341763, 665784, 24532, 33133, 365045, 1288183, 133531, 744926, 742763, 472111, 1012990, 743259, 1707527, 365121, 743270, 666315, 2484270, 471696, 364706, 364741, 80384, 590264, 285693, 173296, 1561035, 469549, 1422194, 731689, 667527, 1860115, 1087348, 204686, 1075949, 378813, 28511, 731422, 813174, 740931, 25883, 310519, 340657, 727289, 743722, 743367, 1662274, 364083, 136399, 731363, 743579, 1088781, 131839, 839101, 2449149, 1846326, 741406, 823590, 724014, 178750, 713623, 767779, 366067, 743078, 383966, 295412, 323438, 364729, 122874, 724642, 795309, 178255, 382760, 135941, 1102600, 69166, 231903, 682276, 1716286, 667533, 40304, 32917, 668452, 731060, 665820, 1624260, 1352408, 471664, 486401, 731722, 135689, 740604, 2139164, 739109, 135671, 666703, 433155, 687800, 502244, 73703, 484577, 1251197, 731202, 381032, 811024, 347503, 238840, 128065, 382791, 1870594, 258114, 731742, 727056, 781738, 487152, 731305, 731726, 342181, 745512, 356960, 365271, 78353, 730924, 742853, 2091591, 731023, 173200, 504596, 666792, 383718, 382002, 741474, 366032, 135766, 383945, 840687, 364436, 729957, 501527, 364686, 1089513, 111460, 127360, 136686, 140405, 1578721, 1735081, 1932202, 2091389, 23939, 251047, 377560, 380394, 384404, 471641, 487103, 487436, 609631, 714216, 723923, 724238, 726763, 729542, 731380, 731598, 738916, 741305, 742965, 742977, 743982, and 744074, 759865,
(iv-2) 50202, 738970, 128694, 741891, 666671, 366388, 146976, 687667, 587186, 667727, 669136, 744001, 666361, 668300, 713647, 2074228, 282587, 179288, 687270, 726830, 666860, 687896, 364510, 324666, 300194, 268972, 135096, 565754, 136361, 300268, 713422, 758037, 713653, 366042, 723914, 172693, 208097, 549054, 730300, 175864, 178908, 687468, 134978, 729956, 503189, 681875, 1104732, 125552, 143759, 1877668, 2139315, 265558, 269873, 301122, 594279, 665376, 667174, 668239, 668536, 668815, 669188, 669564, 687782, 711918, 724276, 725454, 725791, 72744, 730938, 730971, 731751, 738332, 743774, and 745490,
(v) marker genes specific for microfollicular adenoma (FTA-b) that are contained as inserted DNA in the recombinant vectors selected from the group of vectors having the following IMAGE clone ID No:
(v-1) 740931; 731751; 840687; 666128; 379540; 667259; 725585; 503874; 23612; 3054031; 208097; 265558; 136976, 838611, 725707, 594279, 343987, 487474, 731128, 279172, 773330, 1089025, 725502, 2091591, 141216, 235882, 665452, 1251197, 236399, 50491, 376699, 727496, 725877, 682207, 687667, 729924, 179534, 724366, 30471, 743246, 1088781, 345034, 195340, 868577, 2238108, 2148946, 258114, 136919, 1870594, 1241974, 172828, 731348, 740158, 592598, 1326169, 809959, 741790, 725268, 119939, 135980, 136014, 740604, 730037, 448163, 668239, 512420, 258865, 365526, 667365, 742577, 116906, 258747, 738896, 342256, 486221, 288807, 743259, 136223, 725970, 342700, 327182, 264166, 1049033, 365801, 156720, 725473, 1074708, 290265, 687379, 71428, 1624260, 486401, 669564, 133531, 232388, 726454, 1705397, 487848, 809719, 251427, 287843, 1870594, 744926, 682119, 1641901, 742901, 1707527, 742853, 33133, 664233, 504596, 364204, 285693, 25664, 174396, 743722, 209137, 310519, 42280, 249311, 1505308, 727178, 667067, 731726, 46991, 137454, 740347, 666334, 666671, 359461, 487302, 1352408, 504646, 743118, 365919, 687800, 26061, 301122, 122874, 129438, 666235, 471664, 741497, 1117183, 364526, 175772, 179214, 471568, 744606, 1103402, 1942634, 727263, 484577, 745490, 31924, 682555, 665693, 823680, 1626951, 562904, 682311, 132752, 359797, 485803, 725548, 705147, 730012, 504539, 491004, 667440, 1917430, 743774, 666110, 309697, 724416, 135303, 731019, 1287638, 742743, 740718, 1645668, 378813, 1016390, 124701, 162246, 731738, 681890, 309645, 665376, 238840, 489326, 665632, 201467, 188232, 486189, 485085, 203166, 744374, 813174, 1743279, 742763, 156183, 669549, 376356, 665086, 2449149, 137719, 139073, 357442, 745083, 724544, 132933, 665433, 2497617, 22085, 364570, 713879, 731023, 713922, 252291, 1911930, 436554, 743415, 145696, 178348, 667239, 236155, 196992, 2484742, 781738, 210587, 78294, 110938, 114008, 117072, 131839, 153411, 155345, 160163, 178984, 191881, 200018, 234148, 234357, 236386, 239086, 289337, 486201, 486204, 487086, 591534, 627107, 665566, 667144, 713422, 713896, 724042, 725758, 730593, 738315, 741661, 741919, 742965, 743212, 743426, 745018, 812955, 884365, 1056782, 1072914, 1129745, 1131054, 1146624, 1188588, 1420842, 1544175, 1735081, and 2103105,
(v-2) 724609, 365913, 666154, 267358, 665379, 667892, 251250, 713653, 365589, 1087348, 366042, 743146, 174654, 744657, 214008, 666007, 137005, 364436, 738558, 146976, 682175, 725533, 365121, 667112, 770858, 3659591, 469345, 154172, 731404, 730036, 135671, 739097, 739193, 743205, 811582, 364352, 725364, 668684, 725076, 666946, 136260, 971276, 2139164, 365271, 307025, 366254, 125552, 669263, 744439, 26519, 564994, 668815, 725335, 592276, 176543, 296702, 668152, 295412, 149809, 24176, 153667, 726675, 486510, 1526058, 366243, 148796, 194350, 143759, 33621, 668089, 669319, 1173570, 136399, 136686, 208991, 230126, 25883, 31120, 34007, 342551, 364324, 364469, 364569, 364741, 364844, 382612, 383718, 487475, 501476, 502598, 512910, 645455, 665649, 666315, 667514, 667533, 724276, 726353, 726763, 727056, 730938, 731227, 739576, 740130, 740620, 744385, 753215, 78353, 809828, and 813260,
(vi) marker genes specific for follicular carcinoma (FTC) that are contained as inserted DNA in the recombinant vectors selected from the group of vectors having the following IMAGE clone ID No:
(vi-1) 501476, 309009, 739193, 251452, 681957, 739097, 364448, 469549, 202897, 195340, 731380, 2345206, 23765, 471641, 502244, 713653, 486189, 740604, 682207, 740941, 469647, 743519, 743367, 966894, 727056, 2336358, 52897, 1326169, 773330, 139073, 725548, 137238, 742853, 838611, 22908, 207920, 375746, 738896, 152802, 343987, 743246, 1089025, 342256, 729924, 727496, 667514, 50491, 726454, 486401, 2238108, 730123, 342551, 1186334, 365589, 1837488, 1942634, 276727, 666986, 324066, 724014, 364083, 2449149, 744943, 258747, 276658, 265558, 731745, 726763, 28511, 235882, 1707527, 1896337, 448163, 301122, 724306, 687800, 669318, 2214396, 175268, 740347, 365271, 366074, 594279, 1942634, 366289, 665086, 1641901, 724366, 509606, 26061, 162246, 489249, 24541, 723923, 731751, 744410, 1870594, 743259, 731277, 381854, 25949, 489326, 491004, 2728204, 666703, 251147, 1870594, 484535, 258114, 26519, 487148, 726684, 149809, 731616, 252291, 71428, 364324, 156183, 25865, 1705397, 251250, 79729, 365562, 811024, 730926, 1085884, 366159, 682276, 364469, 668442, 741905, 356841, 730814, 177827, 42280, 724151, 743270, 136260,485085, 742837, 666128, 666279, 727092, 364777, 179334, 757248, 2484270, 287843, 469345, 667061, 485803, 727491, 213113, 364846, 743619, 743987, 180640, 364706, 365177, 592598, 971276, 1581686, 713671, 129438, 1117183, 345034, 741066, 1286238, 178792, 740931, 744657, 740381, 2237263, 666154, 665538, 135671, 172996, 1698236, 768241, 188393, 1013595, 1057773, 1129745, 1131054, 1146624, 120649, 124554, 1420842, 153411, 155345, 1899942, 191881, 195712, 236055, 236386, 25077, 30219, 342416, 343867, 35356, 364436, 365121, 38775, 471574, 471834, 490710, 665327, 666928, 667250, 668815, 682451, 687468, 687551, 713896, 724588, 725345, 725696, 725758, 727178, 729489, 739953, 742935, 744897, 80109, and 964736,
(vi-2) 782688, 136014, 135980, 725335, 724150, 307094, 723752, 510318, 726578, 324079, 2139164, 143759, 739237, 152655, 136646, 309697, 384087, 365408, 376699, 134671, 609631, 238840, 1074708, 366254, 742919, 257259, 840753, 668924, 136801, 743245, 731428, 731410, 738507, 430263, 744374, 1492440, 135887, 726782, 379517, 726849, 72744, 2467442, 743426, 135407, 135975, 137454, 238840, 486221, 669574, 266361, 134858, 731073, 359461, 744606, 731060, 666778, 726513, 236155, 744616, 744797, 23827, 743118, 609631, 665376, 742542, 725454, 135689, 713647, 743774, 724416, 687782, 723755, 726092, 731128, 782688, 80688, 53333, 22085, 46991, 486708, 730012, 236119, 549054, 2716748, 744797, 666784, 743043, 238840, 415554, and 729896,
(vii) marker genes specific for multinodular goitres (MNG) that are contained as inserted DNA in the recombinant vectors selected from the group of vectors having the following IMAGE clone ID No:
(vii-1) 284828, 173841, 667527, 202897, 469549, 207920, 469647, 363955, 323438, 276658, 263251, 731277, 35628, 741474, 743394, 212366, 258865, 724014, 742837, 1117183, 276727, 811582, 1743279, 666279, 666703, 725585, 714498, 525221, 739109, 731380, 382738, 1012990, 188232, 1012903, 726675, 356960, 235882, 208439, 178348, 727056, 124781, 300268, 868577, 327182, 249311, 266361, 241481, 376153, 745490, 31924, 235938, 687667, 44881, 725548, 383868, 342551, 382521, 667727, 365562, 359051, 136863, 773286, 236155, 744899, 366042, 383945, 723923, 340657, 668442, 382791, 666794, 121551, 346610, 232366, 110481, 128065, 379937, 731076, 682207, 50202, 725198, 208991, 173371, 724895, 742763, 666426, 743182, 381854, 347503, 743903, 838611, 155345, 2030501, 811024, 489326, 731645, 365521, 135671, 136747, 665433, 1705397, 731689, 724609, 740105, 731389, 1288183, 80162, 127507, 382002, 665086, 213113, 141216, 713913, 813174, 665495, 667110, 744395, 668300, 594279, 668510, 177857, 745512, 382069, 110101, 2242054, 310519, 38403, 3054031, 1188588, 665814, 667756, 173818, 731758, 2345206, 471568, 301122, 730814, 743270, 1859532, 382423, 725677, 744410, 723914, 1846326, 129438, 667514, 823680, 162246, 731115, 125552, 666367, 484535, 2113771, 667252, 744050, 562904, 666554, 667355, 137131, 760065, 727289, 731616, 230126, 725231, 135801, 512420, 594510, 40304, 1641901, 180259, 296702, 485872, 136919, 364741, 2148946, 364436, 136976, 1917430, 1049033, 264166, 741406, 196338, 1088980, 768241, 742061, 69166, 739237, 512910, 42280, 134416, 724238, 357442, 731598, 740347, 730834, 2384812, 724554, 201467, 1308954, 731305, 256177, 157847, 687336, 134615, 73778, 739851, 258966, 136801, 383718, 666334, 486221, 177775, 740457, 713608, 485085, 119772, 31120, 544683, 145696, 383528, 743619, 341763, 471664, 342581, 726647, 743982, 743290, 356841, 178255, 724812, 156183, 152847, 324066, 731742, 724884, 265558, 124701, 376983, 725188, 743016, 140405, 253386, 713879, 238840, 30471, 485803, 179266, 364885, 592276, 486189, 725473, 723950, 724537, 1645668, 137719, 1174287, 743517, 257555, 188393, 745123, 71428, 741497, 544818, 251427, 366085, 25865, 236119, 485104, 1570420, 174654, 745072, 1073288, 1116948, 204381, 255398, 259552, 501476, 645455, 731292, 744881, 745187, 745273, and 768246,
(vii-2) 731202, 179534, 116906 and 1942634, 687625, 726782, 364324, 148796, 744505, 26164, 665668, 713837, 172828, 731726, 151247, 486356, 342181, 471641, 72744, 135689, 740941, 366032, 154172, 743987, 713422, 279172, 131839, 666551, 665392, 2055272, 729957, 258666, 743391, 53333, 109142, 743615, 133531, 309645, 68972, 682311, 666946, 194302, 26455, 744439, 739451, 137010, 669295, 325155, 731685, 172982, 738506, 51232, 78353, 742695, 1690788, 52226, 136114, 153667, 134858, 667587, 1420676, 1716286, 22711, 236399, 2467442, 250463, 250883, 257773, 25883, 295412, 30219, 345034, 486708, 668625, 724366, 725877, 726513, 726849, 730829, 739956, 740604, 741954, 743118, 743774, 744055, 744087, 744505, 744606, 745106, 782688, and 80688,
(viii) marker genes specific for oncocytic adenoma (OTA) that are contained as inserted DNA in the recombinant vectors selected from the group of vectors having the following IMAGE clone ID No:
(viii-1) 80688, 1870594, 731738, 1870594, 365348, 681875, 731751, 136114, 365161, 491004, 724366, 744983, 740718, 884365, 687381, 236399, 731726, 809719, 757248, 172982, 743774, 666410, 743961, 667239, 725502, 265345, 366436, 668584, 725076, 365349, 148796, 23612, 725726, 667361, 53333, 279470, 729510, 725877, 729956, 345034, 665784, 730300, 382612, 137454, 135689, 667310, 1860115, 731714, 365097, 729971, 731128, 682311, 549054, 726092, 609631, 366042, 714437, 68972, 741954, 725791, 730012, 669564, 666455, 238840, 173820, 743485, 726782, 235882, 743760, 364324, 823680, 179334, 729542, 135930, 738506, 155839, 416959, 682109, 175268, 26486, 725707, 725970, 667348, 669263, 28705, 725612, 162246, 666140, 738332, 365877, 740941, 727056, 838611, 731275, 310519, 384087, 174879, 744087, 727496, 379279, 25226, 731428, 365990, 364568, 2266583, 180082, 590264, 725836, 731648, 253386, 151247, 724562, 713837, 379517, 726454, 135887, 682088, 725340, 730606, 840753, 1422791, 723752, 669149, 741891, 669295, 382760, 208097, 665632, 731665, 486708, 285693, 1932168, 726578, 1896337, 250883, 713671, 730037, 740620, 682149, 116906, 669470, 1526058, 365090, 740245, 24541, 1326169, 486935, 365000, 727123, 1877668, 666066, 687306, 503874, 687541, 738944, 726849, 365609, 782688, 38789, 365271, 739901, 145112, 2381770, 742853, 484535, 356841, 743391, 365613, 738970, 364204, 740347, 742806, 152655, 2341829, 112819, 592598, 668089, 739956, 714049, 668684, 293916, 666128, 139242, 51232, 743118, 2728204, 565754, 727229, 2254555, 742543, 364448, 135096, 486401, 364575, 713831, 687990, 668924, 738896, 131839, 207926, 342551, 210587, 324079, 366388, 251216, 137005, 741894, 22085, 44193, 109142, 666361, 148212, 140405, 726272, 448163, 135875, 24392, 725401, 739247, 366289, 868577, 134858, 140347, 261656, 665376, 743246, 1420676, 726596, 357201, 668283, 172828, 487474, 665833, 309645, 376597, 731685, 2484742, 366089, 286138, 687912, 1536968, 666007, 667313, 1641901, 136361, 282587, 665156, 365311, 730123, 173119, 366842, 178750, 1707527, 364510, 364412, 366159, 668707, 279150, 739451, 365408, 745490, 667365, 510318, 2238108, 68950, 743043, 2306096, 740130, 669141, 743722, 22384, 682555, 682251, 669181, 743259, 208991, 342256, 744106, 666425, 738358, 726684, 365562, 745106, 665392, 731060, 687625, 23945, 1239535, 682528, 725268, 486493, 292042, 2467442, 134671, 486356, 665437, 713623, 666029, 379540, 1942634, 252489, 295412, 365121, 594279, 23827, 666517, 682479, 726779, 730829, 21808, 135450, 742100, 34007, 288807, 1016390, 114008, 366254, 713422, 730149, 469549, 135539, 665452, 725394, 665254, 759865, 667174, 744945, 726634, 172785, 2011082, 714013, 33621, 359461, 137131, 134978, 301122, 23271, 366115, 366074, 687270, 687667, 503189, 357807, 743415, 128694, 731398, 180259, 666235, 667353, 682555, 136868, 135991, 687287, 1741589, 52897, 687852, 781738, 26164, 152802, 1089025, 154172, 43541, 24300, 25949, 725677, 214611, 744439, 23872, 2032639, 730938, 743125, 2214396, 727251, 723923, 738938, 134785, 173288, 725364, 26455, 213113, 730036, 687896, 667587, 22908, 731404, 325155, 731423, 46991, 727457, 300194, 669136, 268972, 726513, 667598, 133531, 713653, 30219, 188232, 727263, 53081, 203166, 2237263, 740604, 743987, 738945, 669510, 123950, 26651, 665342, 724892, 687782, 194302, 307025, 727154, 1911930, 469647, 471641, 366032, 29541, 740224, 724150, 136598, 153667, 212366, 666778, 364526, 738966, 3659591, 22711, 1088781, 744505, 744606, 135203, 667061, 365177, 758037, 25883, 713647, 740580, 726353, 178908, 156720, 1844968, 137010, 136801, 724276, 24176, 740381, 137720, 725096, 179288, 665668, 1013595, 1057773, 1101773, 1129745, 123950, 125552, 1256485, 134034, 1352408, 146976, 149910, 1654256, 1737165, 187482, 196115, 214008, 234148, 2361911, 239951, 245426, 248811, 289337, 309009, 324225, 325008, 342416, 364959, 427786, 471101, 530069, 565317, 665881, 667602, 713896, 723753, 724042, 724588, 725511, 726106, 726424, 726904, 727173, 730037, 731152, 73703, 738315, 739953, 740885, 740971, 741835, 742696, 742977, 743237, 744055, 744366, 744413, 744468, 744755, 744756, 744911, and 745125,
(viii-2) 1662274, 145696, 730926, 731115, 155345, 376356, 666794, 739109, 179534, 628418, 741406, 485872, 232388, 667110, 724895, 33133, 730834, 743270, 731380, 713913, 2048801, 485104, 731422, 124781, 731616, 724014, 668186, 731598, 1012990, 382738, 714498, 745072, 1942634, 342581, 347503, 731742, 724537, 365045, 738424, 724556, 730814, 666703, 341763, 739851, 744841, 744895, 726894, 512910, 384404, 382621, 744074, 258127, 364741, 136747, 1174342, 177967, 760065, 177857, 687468, 723950, 2384812, 504646, 740105, 365056, 137478, 378458, 753215, 724642, 731021, 3054031, 178255, 433155, 731076, 744410, 743603, 731722, 742046, 727289, 714493, 178792, 1581686, 1705397, 1308954, 366243, 667252, 731590, 724238, 730924, 504596, 544818, 35628, 486221, 381032, 501527, 258865, 741474, 724884, 276658, 1186334, 731227, 665649, 1859532, 564878, 71428, 666279, 668146, 731241, 257555, 381118, 665814, 726821, 364686, 208439, 82687, 1085884, 727296, 740457, 667527, 1671903, 201467, 135379, 795736, 1075949, 739432, 363955, 213529, 743331, 137238, 743619, 1241974, 632026, 730699, 743451, 665820, 725188, 731202, 379937, 487148, 1288183, 172996, 382069, 731277, 230126, 743982, 743579, 40304, 376983, 179266, 40134, 235938, 731689, 136863, 665433, 727067, 743078, 809959, 381854, 813174, 1846326, 382002, 2030501, 666334, 767779, 731305, 471829, 383868, 566862, 731758, 1624260, 1917430, 364777, 743519, 667355, 364729, 591907, 25865, 241481, 525221, 127507, 307094, 135352, 236119, 743811, 382791, 742061, 323438, 251147, 364706, 487152, 364717, 136686, 364885, 178348, 731363, 666085, 1174287, 667657, 347362, 743268, 196338, 173200, 486189, 665403, 487848, 745512, 731044, 472160, 713624, 177775, 173628, 594655, 744616, 364932, 157847, 744899, 681890, 745133, 713608, 740090, 727178, 743394, 471574, 471664, 486304, 731745, 366585, 1012903, 24587, 742837, 236155, 726600, 152847, 486540, 731019, 985457, 383718, 731645, 724554, 714414, 1578721, 786662, 383966, 261500, 119772, 743182, 366233, 364822, 132752, 364356, 31924, 366085, 38403, 668510, 1743279, 743290, 263251, 30471, 486447, 729929, 2242054, 382521, 296702, 173371, 383945, 121551, 179133, 136919, 666367, 740158, 682585, 1285305, 730942, 284828, 668329, 42280, 136121, 25664, 1561035, 379200, 669549, 668452, 726618, 256177, 724596, 743016, 135671, 177827, 364469, 124701, 485652, 667259, 712118, 346610,743220, 1049033, 430471, 1086823, 1102600, 1103402, 134615, 135630, 156183, 2074228, 236331, 23876, 2484270, 266361, 276727, 340657, 366820, 377560, 384844, 40633, 484577, 485085, 485803, 487436, 502658, 587186, 665086, 666315, 666928, 667258, 669557, 725231, 729875, 731023, 731073, 738916, 739576, 740931, 742542, 742763, 742935, 743517, 744797, 773286, 80384, 811024, 813654, 823590, and 966894,
(ix) marker genes specific for atypical oncocytic adenoma (OTA-aty) that are contained as inserted DNA in the recombinant vectors selected from the group of vectors having the following IMAGE clone ID No:
(ix-1) 745003, 740130, 471641, 682088, 731404, 2254555, 2381770, 214008, 300268, 2467442, 125552, 666517, 366159, 740927, 665784, 24392, 727457, 148796, 590264, 665632, 345034, 840753, 309645, 743043, 731275, 51232, 667048, 838611, 773330, 884365, 667514, 364412, 366115, 757248, 146049, 140347, 342181, 727123, 30219, 155839, 382612, 723923, 682276, 682479, 743118, 740941, 356841, 53081, 745490, 687381, 324079, 173818, 731060, 743367, 365000, 669149, 285693, 731398, 110481, 726578, 687990, 23612, 723752, 713831, 1536968, 725076, 471702, 119939, 448163, 365271, 738506, 665674, 666128, 29541, 204686, 731665, 682251, 724126, 22908, 152802, 356960, 173119, 366289, 669141, 1420676, 682109, 301122, 357442, 174879, 868577, 25883, 288807, 366089, 471852, 162246, 742100, 743722, 666455, 265558, 744943, 379279, 668924, 203166, 194302, 729971, 724366, 682311, 667348, 365877, 731423, 713859, 730037, 23641, 713837, 376597, 135125, 669263, 123950, 172982, 1911930, 152655, 122874, 1084386, 839101, 251427, 666168, 112819, 565317, 1188588, 366889, 743485, 471568, 744505, 669510, 667361, 135689, 666564, 738332, 23765, 668584, 669319, 744087, 188393, 667587, 795309, 292042, 812967, 135450, 327182, 666946, 366032, 1707527, 667310, 52897, 137131, 366042, 738938, 725726, 667313, 665392, 1846326, 730149, 744606, 489249, 739237, 731047, 687541, 365161, 687800, 68950, 136260, 109142, 69166, 357807, 207926, 2091591, 1550616, 740381, 731714, 364575, 1286238, 364526, 151247, 136862, 1855110, 230882, 238840, 26519, 365121, 376462, 379517, 503189, 545475, 730300, 740620, 742935, 744439, 78353, 796513, and 813654,
(ix-2) 724556, 668329, 173200, 174654, 731227, 1624260, 743078, 739576, 3054031, 119772, 487152, 740457, 667527, 382002, 382791, 2384812, 566862, 1075949, 730814, 235938, 745072, 341763, 383718, 486304, 379937, 666279, 241481, 731380, 743270, 1917430, 682585, 666703, 1049033, 1085884, 724642, 665086, 742061, 767779, 729875, 724014, 743603, 714498, 731277, 727289, 744895, 35628, 173200, 743619, 726821, 284828, 129438, 731616, 487103, 667355, 1012990, 487848, 471664, 744899, 42280, 713623, 667110, 502658, 730926, 484577, 1174287, 236155, 381854, 365056, 2030501, 731021, 177967, 667258, 525221, 743331, 135407, 738916, 669557, 382521, 731305, 730401, and 669098,
(x) marker genes specific for oncocytic carcinoma (OTC) that are contained as inserted DNA in the recombinant vectors selected from the group of vectors having the following IMAGE clone ID No:
(x-1) 727123, 724366, 366289, 471641, 501476, 744943, 2237263, 155839, 140347, 743774, 1716286, 365271, 236399, 731423, 252291, 812967, 739097, 471702, 1420676, 739193, 740347, 669564, 609631, 731751, 666671, 745490, 285693, 235882, 725791, 23612, 345034, 503874, 838611, 81911, 469647, 667514, 730012, 730300, 740941, 738358, 486493, 809719, 726684, 687667, 162246, 740620, 366436, 726454, 469549, 146976, 665784, 731738, 365349, 238840, 491004, 122874, 46991, 726763, 78353, 738896, 178908, 416959, 486401, 665632, 742590, 180259, 666455, 22711, 665674, 148796, 489326, 25883, 724306, 745003, 594279, 342256, 26455, 143759, 687852, 592598, 713422, 173820, 136560, 209137, 136361, 743205, 366042, 665538, 727056, 1837488, 782688, 744087, 725877, 2238108, 257259, 725836, 666154, 743246, 971276, 448163, 666410, 137720, 1844968, 73703, 770858, 2055272, 487474, 731023, 667348, 50491, 740130, 342551, 713623, 2032639, 723923, 2728204, 176543, 781738, 839101, 32339, 731357, 738970, 364204, 1089025, 364575, 34007, 135096, 364412, 743259, 1870594, 1101773, 366233, 342290, 366067, 565317, 742853, 29541, 682528, 80688, 731398, 713831, 309697, 744983, 187482, 665452, 665376, 359051, 139242, 1707527, 731726, 145112, 744055, 282587, 136114, 1526058, 366115, 123950, 743043, 416744, 1536968, 309645, 738332, 471696, 365090, 667310, 1088781, 149809, 173119, 724416, 743146, 109142, 1239535, 668815, 364510, 738938, 741954, 668089, 151247, 729542, 665833, 549054, 116906, 666946, 135941, 472111, 809828, 135450, 26651, 2115808, 713671, 743615, 731714, 666128, 366842, 724892, 730938, 22908, 324079, 365613, 231903, 743422, 687541, 668360, 287843, 489249, 342181, 207920, 364324, 682109, 743367, 2381770, 22085, 730123, 135875, 250883, 667061, 724126, 1521706, 687679, 208097, 745106, 38789, 682479, 744606, 668684, 726272, 743451, 25949, 744001, 1870594, 179334, 364568, 357807, 590264, 137353, 207926, 731298, 173288, 565754, 69166, 668584, 364926, 154172, 725726, 725340, 23765, 687625, 743391, 292042, 2032639, 128694, 258666, 382612, 725394, 687381, 469345, 730036, 180640, 188393, 52897, 295412, 51232, 884365, 744945, 174683, 195340, 279470, 731648, 665392, 730606, 379540, 43541, 725364, 731404, 729924, 137454, 665437, 253386, 359202, 731685, 364974, 135689, 727229, 2467442, 1417901, 725707, 204686, 741891, 53081, 134785, 682555, 22384, 134858, 379279, 135539, 300194, 2214396, 687270, 731060, 174861, 366388, 738944, 365288, 251216, 545475, 726779, 136868, 743118, 135352, 180082, 2113771, 178750, 731128, 179733, 137205, 740927, 376153, 365097, 30219, 1089025, 1129745, 131839, 160163, 175268, 1932168, 2349125, 239086, 252489, 267358, 359454, 365121, 427786, 471101, 484504, 487130, 502598, 665668, 665982, 667313, 724588, 725758, 726092, 726353, 739953, 739955, 739956, 740193, 743125, and 812143,
(x-2) 731019, 366085, 258127, 382002, 714414, 486189, 725188, 1241974, 666703, 487152, 40304, 811024, 25865, 666279, 745133, 347362, 258966, 471664, 82687, 740931, 739109, 628418, 731742, 42280, 724014, 1012903, 1288183, 382423, 512910, 667527, 730942, 731021, 3054031, 724596, 179266, 742061, 667683, 743603, 71428, 713608, 173371, 1917430, 745072, 727289, 486221, 740457, 487848, 742542, 723755, 502658, 1012990, 729875, 25664, 731073, 666928, 668452, 486304, 1285305, 235938, 484577, 341763, 382791, 241481, 742837, 258865, 767779, 566862, 725454, 740105, 813654, 1174287, 1049033, 382738, 665433, 823590, 665086, 1645668, 645455, 668239, 741474, 364885, 236119, 35628, 682585, 1075949, 773286, 382069, 213529, 668536, 257555, 31924, 731380, 725231, 727251, 726821, 236155, 740158, 760065, 587186, 40633, 266361, 1085884, 152847, 669188, 363955, 813174, 284828, 134615, 255398, 343987, 731115, 730814, 667252, 174396, 364729, 1103402, 727178, 744899, 2048801, 382521, 39593, 813654, 666928, 175080, 667991, 136933, 723755, 173371, and 1917430,
(xi) marker genes specific for papillary carcinoma (PTC) that are contained as inserted DNA in the recombinant vectors selected from the group of vectors having the following IMAGE clone ID No:
(xi-1) 726763, 739193, 28584, 739097, 25883, 1716286, 365045, 666367, 379200, 365271, 731227, 726760, 384404, 208991, 744943, 1846326, 364729, 730926, 795736, 136686, 667117, 743517, 724151, 296702, 742837, 364777, 364706, 731616, 666986, 80384, 666007, 1837488, 383868, 40304, 366100, 744055, 136919, 230126, 1288183, 813174, 469647, 787938, 740158, 713608, 669318, 795309, 812967, 325155, 382791, 724596, 743703, 724238, 154172, 366484, 667657, 469549, 124781, 668452, 713831, 713913, 383718, 382621, 744616, 364686, 667533, 744074, 364822, 49888, 24587, 363955, 114008, 201467, 295412, 375746, 666315, 744385, 544818, 364846, 145696, 738424, 1624260, 485652, 1285305, 2484270, 194302, 770858, 366074, 823590, 724014, 379937, 194350, 727321, 366243, 68950, 364741, 731305, 1174342, 744895, 342181, 985457, 2384812, 727289, 731277, 1085884, 727296, 383966, 729924, 382738, 471574, 682585, 525221, 1102600, 665379, 753215, 359202, 1581686, 744797, 726513, 743982, 731722, 486304, 323438, 743422, 727491, 364436, 1844968, 666128, 30981, 382069, 3659591, 730829, 433155, 687953, 667176, 1671903, 364324, 726779, 668146, 743579, 26164, 740457, 307025, 667527, 135941, 744657, 665433, 731590, 378458, 723923, 136747, 136399, 744941, 365913, 730924, 376983, 669149, 682251, 2046361, 30471, 665403, 743043, 712118, 153667, 342290, 731422, 666703, 667067, 665814, 742743, 82687, 809828, 173628, 2345206, 471702, 179534, 730814, 172996, 668329, 471641, 665820, 731598, 136121, 148796, 743331, 1942634, 731758, 1352408, 309697, 731202, 472111, 501527, 78353, 741474, 738945, 178750, 743603, 724642, 33621, 364083, 381032, 745003, 366032, 971276, 177967, 609631, 725345, 745512, 71428, 485085, 743903, 744439, 743270, 738966, 196338, 731363, 743394, 366067, 727067, 745106, 31924, 724895, 34007, 744841, 52226, 731021, 682276, 687679, 135352, 1662274, 285693, 307094, 42280, 1308954, 668815, 665668, 232714, 1594414, 682119, 26455, 667110, 134785, 665674, 731380, 180640, 838611, 742046, 30219, 207920, 743016, 667259, 628418, 667252, 504646, 667361, 742061, 726675, 231903, 667892, 136863, 725533, 1917430, 137353, 744106, 714493, 32917, 714013, 342181, 501476, 730834, 79729, 666794, 2467442, 1698236, 665632, 743268, 741066, 726618, 743290, 665495, 73778, 744410, 669149, 381118, 809959, 276658, 632026, 122874, 744050, 731745, 1174287, 745123, 342581, 681890, 136462, 26651, 668186, 740090, 196992, 1578721, 745072, 669263, 729929, 726821, 740941, 1422194, 714414, 173820, 251250, 365056, 724609, 1075949, 512910, 132752, 740105, 2048801, 178255, 364717, 687379, 232388, 342181, 178908, 744604, 714498, 665342, 731241, 487152, 504158, 668476, 743619, 687800, 667355, 724812, 33133, 739432, 730938, 590264, 726634, 687532, 146049, 213529, 687896, 1626951, 284828, 1049033, 341763, 727056, 3054031, 726600, 156183, 731115, 471696, 143759, 366115, 383528, 731742, 31120, 174861, 668584, 743451, 112819, 2030501, 724556, 1101773, 137010, 2345206, 731076, 365000, 1707527, 366233, 366436, 1087348, 668360, 241481, 731404, 21716, 738938, 53333, 383945, 2055272, 682507, 1012990, 180259, 759865, 723950, 1086823, 2048801, 235938, 23876, 2467442, 35628, 366820, 382002, 384844, 430471, 485104, 502598, 667258, 669557, 724225, 738916, 742542, 742935, 767779, and 813260,
(xi-2) 758037, 1932168, 489945, 738970, 667756, 265558, 2113771, 365562, 1712992, 739237, 135539, 159809, 268972, 135303, 1916307, 356841, 682088, 665437, 744945, 2728204, 324079, 727540, 470408, 740347, 364526, 343987, 730300, 594279, 162246, 549054, 743774, 510318, 136976, 725677, 667440, 757248, 140405, 2214396, 436554, 357807, 2306096, 668239, 365919, 665693, 125552, 725585, 823680, 359051, 1846326, 309009, 252291, 1877668, 1505308, 145105, 1741589, 327182, 188393, 743125, 132933, 52897, 731751, 359461, 173841, 212366, 768241, 324066, 730037, 235882, 668300, 2266583, 738358, 723914, 116906, 731738, 209137, 745490, 208097, 44193, 1743279, 110481, 24541, 727251, 745083, 1103402, 681957, 135689, 132935, 731128, 376153, 282587, 666361, 135875, 726636, 1641901, 1690788, 2011082, 366159, 32784, 22711, 713671, 44881, 1492440, 364448, 365526, 725707, 725454, 365348, 1896337, 251427, 725096, 137454, 727092, 357442, 725502, 152655, 668283, 136801, 43541, 667727, 725970, 266361, 135991, 365801, 1238492, 1417901, 731047, 665156, 175772, 141216, 365521, 742919, 743367, 731299, 730012, 489249, 744980, 301122, 713647, 416959, 23612, 128065, 236399, 726335, 366388, 731298, 384087, 300268, 134978, 279470, 740718, 665452, 208439, 738332, 726272, 136014, 135980, 731060, 742100, 364570, 487474, 868577, 1084386, 364412, 249311, 823680, 110101, 1104732, 119939, 1337232, 134858, 135975, 1860115, 188232, 234148, 236331, 251452, 253386, 33285, 33470, 345034, 364204, 364959, 366042, 378813, 379540, 38775, 472114, 486201, 486204, 665376, 669510, 669564, 687667, 705147, 711918, 724366, 725511, 725877, 726092, 726106, 726830, 741587, 741835, 744881, 809719, 812143, 812955, and 966894,
whereby an expression value is provided for each quantified marker gene expression level;
c) comparing (i) the expression value obtained at step b) for each disease-specific marker gene with (ii) a control expression value for the said marker gene, whereby a deregulated expression level of each disease-specific marker gene may be determined;
d) predicting or diagnosing the occurrence of a thyroid disease in the said patient if one or more disease-specific marker genes comprised in a group of marker genes, among the groups (i) to (xi) defined at step b), has a deregulated expression level, and
e) identifying, among the groups of disease-specific marker genes (i) to (xi) defined at step b), the group comprising the said one or more marker genes having a deregulated expression level, whereby the kind of specific thyroid disease that is predicted or diagnosed in the said patient is determined.

2. The integrated in vitro method according to claim 1, wherein, at step b), two or more markers comprised in each group (i) to (xi) are quantified.

3. The integrated in vitro method according to claim 1, wherein, at step b), twelve or more markers comprised in each group (i) to (xi) are quantified.

4. The integrated in vitro method according to claim 1, wherein, at step b), the disease-specific marker genes are selected from two or more groups of marker genes consisting of:
(i) marker genes specific for autoimmune thyroiditis (AT) that are contained as inserted DNA in the recombinant vectors selected from the group of vectors having the following IMAGE clone ID No:
(i-1) 154172, 78353, 135941, 545475, 1087348, 744055, 667174, 592276, 135975, 214008, 295412, 549054, 813260, 565317, 787938, 739956, 1104732, 135630, 667892, 725096, 72744, 645455, 744439, 143759, 669574, 136686, 2266583, 2074228, 364412, 724276, 364959, 669510, 742580, 727251, 300268, 713422, 724416, 31120, 811582, 564994, 1877668, 28584, 2055272, 137720, 687782, 753215, 173200, 486708, 1716286, 503189, 839101, 471641, 472114, 726830, 730938, 744797, 472160, 809828, 2467442, 668186, 669318, 738970, 324079, 33621, 364706, 682088, 591907, 744606, 738332, 51232, 502598, 739193, 741954, 744616, 724596, 364469, 2384812, 34007, 739097, 666778, 667355, 713647, 26519, 731404, 665376, 745106, 744385, 713653, 666007, 725364, 136399, 364729, 23827, 705147, 668452, 669263, 208991, 135407, 742542, 669188, 135689, 738938, 512910, 471574, 22711, 782688, 743118, 726821, 364777, 365045, 740941, 744087, 364324, 309697, 730829, 665649, 136014, 668815, 359461, 366032, 726782, 731073, 1837488, and 238705,
(i-2) 665833, 1645668, 731060, 1705397, 741835, 609631, 2113771, 1337232, 713623, 35356, 122063, 40633, 812143, 811024, 137238, 665881, 140405, 377560, 740193, 727521, 731007, 740604, 665566, 731098, 285666, 255398, 731023, 1286238, 1084386, 730037, 742763, 202897, 726424, 740971, 73703, 120916, 487436, 730968, 471947, 324140, 730032, 666510, 356960, 123950, 485803, 308564, 730971, 1089513, 110938, 201467, 727554, 669503, 730413, 179857, 1899942, 276727, 188393, 234357, 566585, 239951, 36371, 156183, 23121, 471101, 1086914, 21496, 180259, 727173, 342416, 1088781, 486201, 325008, 813174, 2349125, 484504, 2366175, 340657, 366289, 127360, 742574, 744926, 562904, 487103, 743722, 781738, 1632468, 727394, 143957, 251047, 1116948, 2398980, 34783, 1982430, 195712, 1932202, 3058557, 2091389, 5725785, 50491, 366359, 743212, 111460, 741988, 1735081, 825265, 682207, 46991, 1844689, 78294, 258747, 744468, 448163, 724225, 1544175, 742965, 964736, 742839, 851264, 730410, 740931, 152398, 489326, 276658, 1013595, 1737165, 2158322, 162246, 738315, 744413, 727496, 742555, 324225, 134034, 731152, 33285, 486779, 295093, 178984, 725548, 485725, 744366, 159381, 782730, 259552, 503874, 1146624, 174396, 120649, 743113, 744911, 114008, 743237, 1072914, 742899, 153411, 725696, 487130, 155345, 160163, 2137932, 743246, 592598, 490710, 745018, 245426, 1324060, 726454, 740885, 1131054, 191881, 924826, 2238108, 950695, 731762, 714216, 486401, 1654256, 235934, 1089025, 730271, 243741, 742592, 1580147, 668494, 745248, 2361911, 427786, 744755, 726904, 745223, 738896, 743259, 80109, 1712870, 232002, 742696, 342256, 744897, 726557, 741389, 1420842, 725758, 1707527, 289337, 739953, 239086, 713896, 236386, 1129745, 2364998, 504539, 110101, 1057773, 128065, 725345, 1896337, 726156, 731284, 292882, 288807, 152844, 149910, 726147, 152802, 725927, 196115, 1578721, 210587, 742977, 200018, 744756, 741661, 667239, 725473, 2103105, 343867, 364271, 122874, 180950, 232507, 285693, 723938, 665651, 665632, 1326169, 204381, 727207, 665452, 117072, 271985, 1188588, 1929371, 665982, 471210, 824531, 1352408, 667435, 248811, 742649, 741919, 742853, 1541958, 1056782, 668454, 1256485, 49725, 43629, 730865, 667144, 724042, 667602, 745326, 725622, 723753, 724588, 744881, 485085, 745125, 823680, 666755, 155717, 727154, 1870594, 740347, 729489, 345034, 252489, 725268, 124554, 44064, 134615, 379540, 590264, 2108870, 2306096, 80688, 884365, 502658, 1285305, 1641901, 356841, 773286, 868577, 177967, 364204, 204686, 250883, 310519, 743367, 740105, 665784, 724366, 235882, 129438, 38775, 382612, 666410, 486204, 187482, 236155, 137454, 1101773, 743426, 838611, 209137, 666061, 52740, 740617, 2108870, 665651, 1844689, 1352408, 1056782, 668454, 43629, and 155717,
(ii) marker genes specific for Grave's disease (GD that are contained as inserted DNA in the recombinant vectors selected from the group of vectors having the following IMAGE clone ID No:
(ii-1) 724596, 503189, 135407, 731021, 364717, 23817, 24623, 724596, and 180851,
(ii-2) 726424, 122063, 729489, 724588, 343867, 1116948, 727154, 1632468, 1057773, 117072, 73703, 486493, 590264, 726147, 1089513, 724225, 80109, 744385, 1654256, 964736, 667435, 365349, 742696, 295093, 110938, 195712, 245426, 127360, 122874, 448163, 489326, 501476, 202897, 23121, 744911, 743212, 49725, 725548, 191881, 744926, 665452, 2158322, 727394, 811024, 259552, 741389, 232507, 342416, 2238108, 667313, 324225, 366359, 1188588, 742899, 823680, 782730, 204381, 727207, 243741, 120649, 366115, 740885, 726763, 258747, 727554, 153411, 665566, 1286238, 135773, 136686, 731711, 250771, 122063, 744385, 740140, 262894, 53081, 258698, 53119, 136366, 665674, 178161, and 729489,
(iii) marker genes specific for macrofollicular adenoma (FTA-a) that are contained as inserted DNA in the recombinant vectors selected from the group of vectors having the following IMAGE clone ID No:
(iii-1) 486221, 1174287, 666061, 504539, 742837, 173371, 486189, 682585, 258966, 253386, 213529, 566862, 485085, 723755, 382423, 731742, 1285305, 235882, 137478, 731115, 727289, 731047, 236155, 705147, 1049033, 188232, 666794, 382069, 119939, 742763, 731073, 284828, 743603, 665649, 745072, 562904, 40633, 725473, 744616, 356841, 340657, 129438, 485872, 365045, 767779, 587186, 135407, 1645668, 744899, 382521, 382738, 364271, 667110, 682276, 726821, 25664, 21496, 729875, 665086, 178255, 725454, 71428, 382002, 365056, 1016390, 731616, 1578721, 724238, 1012990, 2103105, 2384812, 258865, 341763, 364717, 1116948, 1326169, 741474, 42280, 753215, 1982430, 731277, 31924, 195712, 742542, 665452, 1174287, 487379, and 24065,
(iii-2) 739955, 1420676, 745003, 738938, 300194, 25883, 295412, 364324, 730938, 744055, 725371, 1844968, 22711, 250883, 741954, 744087, 187482, 744505, 1101773, 725364, 742100, 726353, 173820, 782688, 724366, 731726, 154172, 53333, 1716286, 745106, 667313, 208991, 744385, 809828, 135689, 744797, 135941, 366042, 172982, 23827, 549054, 214008, 666007, 136801, 724416, 545475, 667892, 729971, 742580, 740941, 743118, 33621, 364469, 731404, 667348, 78353, 151247, 472160, 1087348, 502598, 744606, 172828, 740130, 667174, 745465, 173820, 24392, 300194, 208991, 24532, 725371, 22711, 376003, 1844968, 359202, 295412, and 25883,
(iv) marker genes specific for atypical follicular adenoma (FTA-aty) that are contained as inserted DNA in the recombinant vectors selected from the group of vectors having the following IMAGE clone ID No:
(iv-1) 208991, 136399, 503874, 123950, 1102600, 377560, 743367, 2048801, 42280, 813174, 30219, 1578721, 33133, 379517, 204686, 743331, 384404, 731598, 725345, 838611, 743982, 379540, 743619, 487848, 738916, 744074, 50491, 1089025, 666128, 740158, 341763, 740941, 729542, 251047, 742965, 140405, 111460, 724642, 723923, 714216, 731616, 309645, 366032, 740105, 744926, 364706, 739097, 1735081, 207920, 487436, 739193, 177857, 1089513, 743603, 2091389, 665784, 731422, 71428, 179534, 1012990, 666315, 1932202, 713831, 760065, 731380, 665632, 731202, 740604, 665086, 136686, 726763, 743270, 759865, 364741, 823590, 609631, 730926, 201467, 310519, 127360, 471641, 742977, 80384, 724238, 378813, 35628, 487103, 28584, 1075949, 731305, 503874, 741305, 208991, 743367, 380394, 123950, 23939, 714196, and 1102600,
(iv-2) 687667, 265558, 738970, 726830, 146976, 745490, 282587, 366388, 2074228, 669188, 668536, 366042, 208097, 743774, 713647, 134978, 731751, 300194, 713653, 724276, 301122, 730300, 669564, 668239, 738332, 713422, 594279, 1104732, 665376, 730938, 667174, 1877668, 300268, 587186, 711918, 72744, 1932168, 143759, 725791, 730971, 687468, 125552, 668815, 687782, 725454, 2074228, 146976, 2139315, 726830, 50202, 269873, 745490, and 265558,
(v) marker genes specific for microfollicular adenoma (FTA-b) that are contained as inserted DNA in the recombinant vectors selected from the group of vectors having the following IMAGE clone ID No:
(v-1) 740931, 740604, 172828, 725268, 136014, 731751, 594279, 731128, 379540, 725877, 743774, 136976, 178984, 208097, 627107, 209137, 741587, 236399, 234148, 129438, 23612, 234357, 1088781, 743118, 359461, 666128, 838611, 200018, 265558, 288807, 239086, 137454, 1735081, 742965, 160163, 1146624, 725707, 110938, 153411, 343987, 155345, 289337, 117072, 191881, 309697, 738315, 727496, 1056782, 486201, 665566, 119939, 25664, 1420842, 725758, 1129745, 713896, 175772, 345034, 364204, 669564, 743426, 1072914, 78294, 743259, 486204, 884365, 504539, 378813, 745018, 740158, 687667, 236386, 741661, 562904, 1641901, 741919, 1188588, 592598, 1870594, 114008, 809719, 2103105, 713422, 1705397, 1089025, 705147, 730037, 667144, 503874, 484577, 812955, 744606, 1016390, 487848, 251427, 1544175, 726147, 682207, 2364998, 731738, 725548, 1049033, 210587, 1131054, 744926, 724042, 743246, 743212, 131839, 823680, 740604, 487086, 730593, 591534, and 743118,
(v-2) 667892, 267358, 666007, 146976, 486510, 564994, 364436, 365271, 739193, 666315, 592276, 739097, 713653, 125552, 725364, 214008, 731404, 1087348, 296702, 251250, 469345, 669263, 727056, 744385, 731277, 512910, 34007, 744439, 365121, 230126, 811582, 366042, 33621, 730938, 665649, 668815, 740620, 502598, 726763, 364741, 208991, 136399, 25883, 136686, 383718, 364324, 724276, 726353, 382612, 295412, 645455, 501476, 739576, 143759, 740130, 31120, 78353, 731227, 26519, 364469, 813260, 342551, 809828, 753215, 364844, 564994, 740620, 267358, 667533, 666315, 666007, 1173570, 364569, 667514, and 487475,
(vi) marker genes specific for follicular carcinoma (FTC) that are contained as inserted DNA in the recombinant vectors selected from the group of vectors having the following IMAGE clone ID No:
(vi-1) 726684, 501476, 38775, 251250, 669318, 35356, 364469, 713653, 739193, 26519, 30219, 155345, 251452, 739953, 202897, 490710, 739097, 469345, 365121, 309009, 364436, 725345, 153411, 744897, 725758, 485803, 175268, 120649, 364777, 191881, 276658, 471574, 1129745, 742935, 343867, 713896, 236386, 276727, 342416, 124554, 724588, 1057773, 1420842, 666928, 727178, 1013595, 725548, 207920, 235882, 964736, 489326, 1146624, 1131054, 469647, 725696, 740941, 668815, 1705397, 687468, 195712, 342551, 743367, 80109, 1899942, 729489, 682451, 687551, 236055, 25077, 665327, 667250, 471834, 26519, and 365121,
(vi-2) 72744, 2467442, 726782, 743426, 136801, 135407, 135975, 137454, 726849, 238840, 324079, 486221, 669574, 266361, 134858, 731073, 359461, 744606, 731060, 666778, 726513, 236155, 744616, 744797, 23827, 743118, 609631, 143759, 665376, 742542, 725454, 135689, 713647, 743774, 136014, 724416, 687782, 723755, 726092, 731128, 782688, 80688, 53333, 22085, 46991, 486708, 730012, 236119, 549054, 2716748, 744797, 666784, 743043, 238840, 415554, and 729896,
(vii) marker genes specific for multinodular goitres (MNG) that are contained as inserted DNA in the recombinant vectors selected from the group of vectors having the following IMAGE clone ID No:
(vii-1) 811582, 188232, 682207, 356960, 236155, 501476, 773286, 173371, 276727, 30471, 645455, 745273, 744881, 1705397, 741474, 667527, 207920, 512910, 255398, 110101, 723923, 259552, 129438, 363955, 266361, 235882, 253386, 364885, 740105, 204381, 742837, 134615, 1116948, 136801, 1073288, 207920, 731292, 768246, 594510, and 745187,
(vii-2) 471641, 713422, 740941, 72744, 309645, 744505, 731726, 726782, 135689, 665668, 2055272, 151247, 366032, 172982, 743118, 364324, 745106, 172828, 740604, 744055, 154172, 131839, 53333, 744087, 726513, 295412, 134858, 30219, 739956, 109142, 486708, 78353, 725877, 726849, 782688, 345034, 25883, 2467442, 22711, 743774, 741954, 744606, 1420676, 724366, 1716286, 80688, 236399, 250883, 730829, 250463, 665668, 486356, 257773, 726782, 744505, 135689, 731726, 668625, 309645, and 740941,
(viii) marker genes specific for oncocytic adenoma (OTA) that are contained as inserted DNA in the recombinant vectors selected from the group of vectors having the following IMAGE clone ID No:
(viii-1) 80688, 666410, 725877, 729971, 236399, 726092, 724366, 172982, 1870594, 742100, 366388, 366042, 744983, 365349, 731275, 416959, 53333, 151247, 731726, 345034, 382612, 809719, 731738, 730012, 667348, 173820, 667313, 739247, 137454, 744087, 730300, 884365, 135689, 743774, 725707, 22085, 1860115, 1932168, 250883, 726849, 669564, 2266583, 139242, 740941, 729542, 364324, 665376, 665784, 51232, 731751, 175268, 723753, 609631, 238840, 669263, 252489, 486708, 666066, 730829, 33621, 23612, 667239, 740620, 731128, 739956, 725791, 726684, 486401, 23827, 365271, 365121, 134858, 782688, 741835, 590264, 471641, 738332, 379517, 726454, 726782, 731404, 2032639, 741954, 366115, 366289, 727154, 725268, 740130, 745106, 743125, 665668, 549054, 282587, 146976, 109142, 253386, 1101773, 131839, 214008, 669510, 665833, 162246, 743118, 738970, 310519, 742853, 726106, 30219, 682088, 724588, 745125, 364204, 172828, 666007, 727496, 740971, 823680, 1707527, 565317, 1641901, 309645, 666128, 1057773, 667174, 285693, 309009, 743259, 666778, 731152, 740347, 1896337, 665632, 379540, 757248, 134978, 1420676, 210587, 726424, 592598, 665881, 725511, 738896, 740885, 295412, 687782, 73703, 744756, 288807, 180259, 137131, 838611, 724042, 744366, 364412, 427786, 448163, 34007, 325008, 742977, 471101, 1256485, 730037, 742696, 713831, 725622, 486493, 744413, 724276, 725096, 234148, 239951, 134034, 868577, 713623, 366074, 149910, 1877668, 154172, 289337, 726513, 2306096, 726904, 364959, 727251, 744505, 667602, 342256, 1326169, 744055, 187482, 196115, 731060, 342416, 743237, 123950, 125552, 503874, 781738, 1654256, 743246, 740604, 739953, 744911, 364526, 503189, 738938, 208097, 25883, 324225, 245426, 723923, 248811, 738315, 743722, 1013595, 1737165, 727173, 744755, 140405, 300194, 744468, 2361911, 46991, 1352408, 1129745, 1089025, 745223, 713896, 725877, 530069, 724366, 666410, 729971, and 726849,
(viii-2) 258865, 485104, 430471, 730926, 731616, 365045, 485872, 724895, 179534, 486189, 731115, 364686, 731076, 743270, 178255, 33133, 366820, 365056, 666334, 666703, 1086823, 364741, 666794, 137478, 177857, 42280, 31924, 235938, 2484270, 213529, 724014, 2048801, 731380, 743331, 730814, 1174287, 667252, 731598, 136863, 744895, 382738, 667110, 471574, 23876, 760065, 1085884, 745072, 2384812, 724238, 731422, 744899, 666279, 341763, 731305, 665433, 740105, 236119, 30471, 384844, 665086, 364885, 383868, 364717, 714498, 744074, 512910, 382621, 665649, 379937, 364706, 743982, 743619, 587186, 1705397, 741474, 241481, 2030501, 433155, 230126, 35628, 363955, 1012990, 71428, 525221, 731202, 667527, 364729, 743603, 173371, 753215, 471664, 742837, 668186, 687468, 381854, 738916, 1075949, 201467, 487152, 767779, 152847, 40304, 740457, 486221, 382791, 591907, 566862, 364777, 744616, 742935, 724642, 257555, 1049033, 667258, 382521, 731021, 384404, 742061, 382069, 731277, 727289, 173200, 727178, 795736, 177967, 668452, 823590, 382002, 726821, 813174, 725231, 731742, 731227, 1917430, 667355, 135630, 383718, 487848, 729875, 813654, 740158, 1624260, 773286, 284828, 729929, 731073, 731023, 136686, 472160, 484577, 486304, 740931, 377560, 25664, 669557, 811024, 1285305, 502658, 276727, 236155, 666928, 1103402, 682585, 966894, 134615, 487436, 296702, 1578721, 485803, 1102600, 364469, 739576, 742542, 2074228, 743517, 340657, 156183, 742763, 485085, 80384, 266361, 40633, 175772, 666315, 744797, and 236331,
(ix) marker genes specific for atypical oncocytic adenoma (OTA-aty) that are contained as inserted DNA in the recombinant vectors selected from the group of vectors having the following IMAGE clone ID No:
(ix-1) 26519, 172982, 744439, 745003, 238840, 1420676, 742935, 503189, 151247, 214008, 365121, 730300, 25883, 545475, 738332, 669263, 125552, 744087, 740620, 366042, 884365, 78353, 109142, 742100, 590264, 379517, 667313, 813654, 730037, 135689, 740130, 742935, 1855110, 796513, 376462, 230882, 813654, and 136862,
(ix-2) 487152, 740457, 667527, 382002, 382791, 2384812, 566862, 1075949, 730814, 235938, 745072, 341763, 383718, 486304, 379937, 666279, 241481, 731380, 743270, 1917430, 682585, 666703, 1049033, 1085884, 724642, 665086, 742061, 767779, 729875, 724014, 743603, 714498, 731277, 727289, 744895, 35628, 173200, 743619, 726821, 284828, 129438, 731616, 487103, 667355, 1012990, 487848, 471664, 744899, 42280, 713623, 667110, 502658, 730926, 484577, 1174287, 236155, 381854, 365056, 2030501, 731021, 177967, 667258, 525221, 743331, 135407, 738916, 669557, 382521, 731305, 730401, 713623, 235938, 669098, 1049033, 730814, 382002, 667527, 740457, 382791, and 665086,
(x) marker genes specific for oncocytic carcinoma (OTC) that are contained as inserted DNA in the recombinant vectors selected from the group of vectors having the following IMAGE clone ID No:
(x-1) 782688, 78353, 666410, 740130, 745003, 740620, 731726, 1716286, 250883, 1420676, 740941, 135689, 744087, 724366, 109142, 2055272, 486493, 590264, 238840, 713623, 469345, 80688, 738970, 725791, 364412, 366042, 146976, 739955, 1844968, 365271, 187482, 668815, 744606, 501476, 180259, 1101773, 809828, 731404, 366115, 726353, 713422, 173820, 137454, 665668, 2032639, 366289, 726763, 549054, 743367, 135941, 131839, 46991, 724416, 282587, 503874, 744983, 209137, 502598, 667348, 812143, 745106, 730012, 726092, 739193, 726684, 175268, 740347, 471101, 151247, 365121, 345034, 252489, 364324, 565317, 34007, 162246, 253386, 731060, 738896, 743125, 743774, 137720, 739956, 741954, 139242, 739097, 309645, 73703, 342256, 609631, 667313, 51232, 745490, 740193, 469647, 486401, 22711, 484504, 2349125, 665376, 724588, 154172, 267358, 739953, 838611, 427786, 1129745, 725758, 730300, 1089025, 729542, 1932168, 687667, 309697, 471641, 487130, 731738, 545475, 160163, 365349, 239086, 665982, 366388, 25883, 78353, 359454, 666410, 740620, 740347, and 364412,
(x-2) 40304, 382423, 173371, 1917430, 745072, 727289, 486221, 740457, 742061, 512910, 487848, 742542, 258966, 723755, 502658, 471664, 1012990, 729875, 25664, 487152, 724596, 731073, 666928, 668452, 486304, 382002, 1285305, 235938, 484577, 341763, 382791, 241481, 742837, 258865, 767779, 566862, 725454, 740105, 731021, 813654, 1174287, 71428, 1049033, 382738, 665433, 823590, 665086, 1645668, 645455, 668239, 741474, 364885, 236119, 35628, 682585, 1075949, 773286, 382069, 213529, 668536, 257555, 42280, 31924, 731380, 725231, 727251, 726821, 236155, 740158, 760065, 587186, 40633, 486189, 731742, 266361, 1085884, 152847, 669188, 363955, 724014, 813174, 284828, 134615, 255398, 343987, 731115, 730814, 667252, 740931, 743603, 174396, 364729, 1103402, 727178, 744899, 2048801, 382521, 811024, 39593, 813654, 666928, 175080, 667991, 136933, 723755, 173371, 742061, 471664, and 1917430,
(xi) marker genes specific for papillary carcinoma (PTC) that are contained as inserted DNA in the recombinant vectors selected from the group of vectors having the following IMAGE clone ID No:
(xi-1) 1624260, 743517, 28584, 296702, 80384, 669557, 731227, 795736, 787938, 384844, 384404, 502598, 1086823, 382621, 1716286, 823590, 731422, 23876, 25883, 667258, 364729, 433155, 669318, 738916, 727289, 731598, 739097, 383718, 364777, 667527, 366032, 744074, 666315, 724596, 724014, 742935, 1102600, 743982, 744616, 724642, 726763, 30471, 726513, 2384812, 309697, 740457, 730926, 666007, 382069, 135941, 742061, 731616, 78353, 813174, 485104, 2484270, 744439, 724238, 740158, 382791, 208991, 731305, 35628, 179534, 731021, 364324, 726821, 666703, 665668, 365045, 668452, 809828, 382738, 201467, 363955, 730814, 739193, 731277, 744797, 366820, 383868, 682585, 382002, 745072, 743270, 742837, 40304, 1917430, 813260, 744055, 379937, 136686, 1012990, 1285305, 486304, 665433, 742542, 731202, 1075949, 668815, 744895, 2048801, 744385, 42280, 667110, 31924, 284828, 364706, 177967, 71428, 364686, 730829, 469647, 753215, 33133, 724225, 738938, 430471, 235938, 767779, 731380, 1578721, 2467442, and 1624260,
(xi-2) 359051, 343987, 301122, 594279, 745490, 711918, 687667, 378813, 265558, 162246, 812955, 145105, 757248, 324079, 741587, 740347, 33470, 208097, 743125, 364526, 136976, 188393, 1641901, 140405, 627107, 730012, 725511, 359461, 726106, 744881, 486204, 668239, 823680, 725707, 356841, 251427, 236331, 1877668, 134978, 309009, 376153, 234148, 731128, 731047, 727251, 1896337, 38775, 743367, 2306096, 23612, 125552, 743774, 236399, 2266583, 682088, 966894, 175772, 282587, 738970, 251452, 738332, 812143, 1103402, 2113771, 868577, 731738, 549054, 713647, 379540, 235882, 1932168, 725096, 472114, 731060, 128065, 1860115, 33285, 135689, 266361, 137454, 730300, 136014, 742100, 731751, 416959, 209137, 22711, 1104732, 300268, 669564, 730037, 725877, 705147, 364204, 366388, 726830, 119939, 110101, 364959, 486201, 253386, 665376, 364412, 188232, 725454, 726092, 809719, 741835, 135975, 345034, 1337232, 724366, 136801, 669510, 366042, 134858, 727251, 627107, 376153, 743125, 140405, 823680, 136976, 324079, 378813, 687667, and 265558.

5. The integrated in vitro method according to claim 1, wherein, at step b), the disease-specific marker genes are selected from two or more groups of marker genes consisting of:
(i) marker genes specific for autoimmune thyroiditis (AT) that are contained as inserted DNA in the recombinant vectors selected from the group of vectors having the following IMAGE clone ID No:
(i-1) 669510, 725096, 366100, 325155, 154172, 787938, 744055, 592276, 740941, 73778, 667174, 739193, 295412, 23827, 136686, 744385, 471641, 724276, 28584 and 268972,
(i-2) 43541, 687990, 365990, 562904, 725548, 365877, 742763, 180259, 2449149, 1089025, 713623, 382760, 258747, 743961, 174396, 724306, 1505308, 726454, 665452 and 665833,
(ii) marker genes specific for Grave's disease (GD that are contained as inserted DNA in the recombinant vectors selected from the group of vectors having the following IMAGE clone ID No:
(ii-1) 25664, 868169, 731380, 1049033, 730942, 731616, 726578, 366388, 485872, 1662274, 2074228, 730926, 745106, 731021, 207920, 258865, 503189, 745490, 1103402 and 1911930,
(ii-2) 687990, 743961, 486493, 379279 and 667313,
(iii) marker genes specific for macrofollicular adenoma (FTA-a) that are contained as inserted DNA in the recombinant vectors selected from the group of vectors having the following IMAGE clone ID No:
(iii-1) 2348237, 135379, 743851, 1712992, 376699, 1251197, 1174287, 173841, 486221, 265345, 666564, 252382, 173371, 731073, 253386, 1285305, 342700, 179214, 3054031 and 359797, 745003, 1844968, 364324, 357201, 24392, 1420676, 687532, 724562, (iii-2) 729510, 725371, 24083, 738938, 730938, 148796, 137205, 174683, 21808, 3659591, 325155 and 666792,
(iv) marker genes specific for atypical follicular adenoma (FTA-aty) that are contained as inserted DNA in the recombinant vectors selected from the group of vectors having the following IMAGE clone ID No:
(iv-1) 503874, 666128, 838611, 208991, 739097, 1089025, 725548, 739193, 155839, 28584, 202897, 743331, 743619, 725345, 345034, 235938, 729924, 681957, 30219 and 773330,
(iv-2) 50202, 738970, 128694, 741891, 666671, 366388, 146976, 687667, 587186, 667727, 669136, 744001, 666361, 668300, 713647, 2074228, 282587, 179288, 687270 and 726830,
(v) marker genes specific for microfollicular adenoma (FTA-b) that are contained as inserted DNA in the recombinant vectors selected from the group of vectors having the following IMAGE clone ID No:
(v-1) 740931; 731751; 840687; 666128; 379540; 667259; 725585; 503874; 23612; 3054031; 208097; 265558; 136976, 838611, 725707, 594279, 343987, 487474, 731128, 279172, 773330,
(v-2) 724609, 365913, 666154, 267358, 665379, 667892, 251250, 713653, 365589, 1087348, 366042, 743146, 174654, 744657, 214008, 666007, 137005, 364436, 738558 and 146976,
(vi) marker genes specific for follicular carcinoma (FTC) that are contained as inserted DNA in the recombinant vectors selected from the group of vectors having the following IMAGE clone ID No:
(vi-1) 501476, 309009, 739193, 251452, 681957, 739097, 364448, 469549, 202897, 195340, 731380, 2345206, 23765, 471641, 502244, 713653, 486189, 740604, 682207 and 740941,
(vi-2) 782688, 136014, 135980, 725335, 724150, 307094, 723752, 510318, 726578, 324079, 2139164, 143759, 739237, 152655, 136646, 309697, 384087, 365408, 376699 and 134671
(vii) marker genes specific for multinodular goitres (MNG) that are contained as inserted DNA in the recombinant vectors selected from the group of vectors having the following IMAGE clone ID No:
(vii-1) 284828, 173841, 667527, 202897, 469549, 207920, 469647, 363955, 323438, 276658, 263251, 731277, 35628, 741474, 743394, 212366, 258865, 724014, 742837 and 1117183,
(vii-2) 687625, 726782, 364324, 148796, 744505, 26164, 665668, 713837, 172828, 731726, 151247, 486356, 342181, 471641, 72744, 135689, 740941, 366032 and 154172,
(viii) marker genes specific for oncocytic adenoma (OTA) that are contained as inserted DNA in the recombinant vectors selected from the group of vectors having the following IMAGE clone ID No:
(viii-1) 80688, 1870594, 731738, 1870594, 365348, 681875, 731751, 136114, 365161, 491004, 724366, 744983, 740718, 884365, 687381, 236399, 731726, 809719, 757248 and 172982,
(viii-2) 1662274, 145696, 730926, 731115, 155345, 376356, 666794, 739109, 179534, 628418, 741406, 485872, 232388, 667110, 724895, 33133, 730834, 743270, 731380 and 713913,
(ix) marker genes specific for atypical oncocytic adenoma (OTA-aty) that are contained as inserted DNA in the recombinant vectors selected from the group of vectors having the following IMAGE clone ID No:
(ix-1) 745003, 740130, 471641, 682088, 731404, 2254555, 2381770, 214008, 300268, 2467442, 125552, 666517, 366159, 740927, 665784, 24392, 727457, 148796, 590264 and 665632,
(ix-2) 724556, 668329, 173200, 174654, 731227, 1624260, 743078, 739576, 3054031 and 119772,
(x) marker genes specific for oncocytic varcinoma (OTC) that are contained as inserted DNA in the recombinant vectors selected from the group of vectors having the following IMAGE clone ID No:
(x-1) 727123, 724366, 366289, 471641, 501476, 744943, 2237263, 155839, 140347, 743774, 1716286, 365271, 236399, 731423, 252291, 812967, 739097, 471702, 1420676 and 739193,
(x-2) 731019, 366085, 258127, 382002, 714414, 486189, 725188, 1241974, 666703, 487152, 40304, 811024, 25865, 666279, 745133, 347362, 258966, 471664, 82687 and 740931
(xi) marker genes specific for papillary carcinoma (PTC) that are contained as inserted DNA in the recombinant vectors selected from the group of vectors having the following IMAGE clone ID No:
(xi-1) 726763, 739193, 28584, 739097, 25883, 1716286, 365045, 666367, 379200, 365271, 731227, 726760, 384404, 208991, 744943, 1846326, 364729, 730926, 795736 and 136686,
(xi-2) 758037, 1932168, 489945, 738970, 667756, 265558, 2113771, 365562, 1712992, 739237, 135539, 159809, 268972, 135303, 1916307, 356841, 682088, 665437, 744945 and 2728204.

6. The integrated in vitro method according to claim 1, wherein, at step b), the disease-specific marker genes are selected from two or more groups of marker genes consisting of:
(i) marker genes specific for autoimmune thyroiditis (AT) that are contained as inserted DNA in the recombinant vectors selected from the group of vectors having the following IMAGE clone ID No:
(i-1) 1837488, 284828, 729542, and 238705,
(i-2) 52740, 208097, 36371, 740617, 1086914, 2108870, 665651, 1844689, 448163, 1929371, 1352408, 742649, 1056782, 668454, 43629, and 155717,
(ii) marker genes specific for Grave's disease (GD that are contained as inserted DNA in the recombinant vectors selected from the group of vectors having the following IMAGE clone ID No:
(ii-1) 23817, 24623, 724596, and 180851,
(ii-2) 135773, 136686, 731711, 250771, 122063, 744385, 740140, 262894, 53081, 258698, 53119, 136366, 665674, 726424, 178161, and 729489,
(iii) marker genes specific for macrofollicular adenoma (FTA-a) that are contained as inserted DNA in the recombinant vectors selected from the group of vectors having the following IMAGE clone ID No:
(iii-1) 1174287, 487379, and 24065,
(iii-2) 745465, 173820, 24392, 300194, 208991, 24532, 725371, 745003, 738938, 739955, 22711, 376003, 1844968, 359202, 295412, 1420676, and 25883,
(iv) marker genes specific for atypical follicular adenoma (FTA-aty) that are contained as inserted DNA in the recombinant vectors selected from the group of vectors having the following IMAGE clone ID No:
(iv-1) 503874, 741305, 208991, 136399, 743367, 380394, 123950, 23939, 714196, and 1102600,
(iv-2) 2074228, 146976, 738970, 2139315, 726830, 50202, 269873, 745490, 265558, and 687667,
(v) marker genes specific for microfollicular adenoma (FTA-b) that are contained as inserted DNA in the recombinant vectors selected from the group of vectors having the following IMAGE clone ID No:
(v-1) 740931, 740604, 487086, 172828, 730593, 591534, and 743118,
(v-2) 364844, 564994, 740620, 267358, 667533, 666315, 146976, 666007, 667892, 1173570, 364569, 667514, and 487475,
(vi) marker genes specific for follicular carcinoma (FTC) that are contained as inserted DNA in the recombinant vectors selected from the group of vectors having the following IMAGE clone ID No:
(vi-1) 726684, 682451, 687551, 236055, 25077, 665327, 667250, 471834, 26519, and 365121,
(vi-2) 2716748, 744797, 666784, 743043, 238840, 415554, 72744, 729896, 743426, and 2467442,
(vii) marker genes specific for multinodular goitres (MNG) that are contained as inserted DNA in the recombinant vectors selected from the group of vectors having the following IMAGE clone ID No:
(vii-1) 1073288, 207920, 731292, 768246, 594510, and 745187,
(vii-2) 250463, 665668, 486356, 713422, 257773, 726782, 744505, 72744, 135689, 731726, 668625, 309645, 740941, and 471641,
(viii) marker genes specific for oncocytic adenoma (OTA) that are contained as inserted DNA in the recombinant vectors selected from the group of vectors having the following IMAGE clone ID No:
(viii-1) 725877, 530069, 724366, 666410, 729971, 80688, and 726849,
(viii-2) 365934, 753215, 730926, 485872, 365045, 724895, 179534, 486189, 53341, 485104, 731616, 430471, and 258865,
(ix) marker genes specific for atypical oncocytic adenoma (OTA-aty) that are contained as inserted DNA in the recombinant vectors selected from the group of vectors having the following IMAGE clone ID No:
(ix-1) 26519, 742935, 1855110, 796513, 376462, 230882, 813654, and 136862,
(ix-2) 730401, 713623, 235938, 669098, 1049033, 487152, 730814, 382002, 667527, 740457, 382791, and 665086,
(x) marker genes specific for oncocytic varcinoma (OTC) that are contained as inserted DNA in the recombinant vectors selected from the group of vectors having the following IMAGE clone ID No:
(x-1) 78353, 782688, 359454, 666410, 740620, 740347, and 364412,
(x-2) 39593, 813654, 666928, 175080, 667991, 136933, 723755, 173371, 742061, 471664, 40304, 382423, and 1917430,
(xi) marker genes specific for papillary carcinoma (PTC) that are contained as inserted DNA in the recombinant vectors selected from the group of vectors having the following IMAGE clone ID No:
(xi-1) 1624260,
(xi-2) 729542, 727251, 627107, 376153, 743125, 140405, 823680, 136976, 324079, 378813, 711918, 208097, 687667, 359051, 343987, 745490, 265558, 301122, and 594279.

7. The integrated in vitro method according to claim 1, wherein, at step b), the disease-specific marker genes are selected from two or more groups of marker genes consisting of:
(i) marker genes specific for autoimmune thyroiditis (AT) that are contained as inserted DNA in the recombinant vectors selected from the group of vectors having the following IMAGE clone ID No:
(i-1) 1174287, 119772, 133531, 135689, 136919, 139242, 1420676, 148796, 154172, 155345, 1716286, 172982, 173841, 1846326, 1877668, 208991, 214008, 2266583, 22711, 2384812, 258127, 25883, 258865, 265558, 28584, 295412, 301122, 309645, 324079, 325155, 343987, 359051, 359461, 364324, 364412, 365045, 365121, 365271, 365589, 365913, 366032, 366042, 366100, 38403, 471641, 471664, 487152, 512910, 545475, 549054, 587186, 592276, 628418, 665086, 665376, 666154, 666361, 667174, 667348, 667892, 668684, 681875, 687667, 714437, 724562, 724892, 725076, 726596, 727056, 727251, 727289, 72744, 729510, 730012, 730300, 731060, 731115, 731726, 738332, 738970, 739097, 739193, 740457, 740941, 743774, 744087, 744439, 745003, 745072, 745490, 782688, 78353 and 787938,
(i-2) 1088781, 1089513, 1131054, 116906, 122874, 123950, 1251197, 1256485, 1286238, 1326169, 1352408, 140405, 1578721, 162246, 1705397, 1707527, 174396, 180259, 1870594, 188393, 1942634, 201467, 204686, 210587, 2238108, 245426, 250883, 2728204, 279470, 287843, 3054031, 310519, 342256, 345034, 356841, 356960, 366289, 379200, 382760, 427786, 448163, 46991, 485803, 486401, 489326, 590264, 592598, 609631, 665452, 666410, 667239, 668584, 682207, 687990, 713623, 725473, 725548, 726454, 727496, 730037, 731751, 738944, 740347, 740931, 742696, 742853, 743246, 743367, 744911, 80688, 811024, 813174, 823680 and 884365,
(ii) marker genes specific for Grave's disease (GD that are contained as inserted DNA in the recombinant vectors selected from the group of vectors having the following IMAGE clone ID No:
(ii-1) 145696, 151247, 1662274, 173296, 341763, 486189, 594279, 730926, 731616 and 740620,
(iii) marker genes specific for macrofollicular adenoma (FTA-a) that are contained as inserted DNA in the recombinant vectors selected from the group of vectors having the following IMAGE clone ID No:
(iii-2) 136114, 364575 and 366436,
(iii-1) 1188588, 137131, 137454, 173371, 235882, 235938, 253386, 265345, 276727, 42280, 486221, 666703, 682276, 725707, 726600, 741406, 743259, 744983 and 838611,
(iv) marker genes specific for atypical follicular adenoma (FTA-aty) that are contained as inserted DNA in the recombinant vectors selected from the group of vectors having the following IMAGE clone ID No:
(iv-1) 1089025, 1536698, 155839, 238840, 30219, 33133, 378813, 379517, 379540, 40304, 665784, 666128, 667527, 724014, 739109, 740105, 740604 and 743270,
(iv-2) 208097, 669136 and 687468,
(v) marker genes specific for microfollicular adenoma (FTA-b) that are contained as inserted DNA in the recombinant vectors selected from the group of vectors having the following IMAGE clone ID No:
(v-1) 1641901, 23612, 236399, 491004, 667365, 724366, 725877, 731128, 731738 and 809719,
(vi) marker genes specific for follicular carcinoma (FTC) that are contained as inserted DNA in the recombinant vectors selected from the group of vectors having the following IMAGE clone ID No:
(vi-1) 179334, 713671, 726684 and 757248,
(vii) marker genes specific for multinodular goitres (MNG) that are contained as inserted DNA in the recombinant vectors selected from the group of vectors having the following IMAGE clone ID No:
(vii-1) 1012990, 208439 and 36521,
(vii-2) 194302,
(viii) marker genes specific for oncocytic adenoma (OTA) that are contained as inserted DNA in the recombinant vectors selected from the group of vectors having the following IMAGE clone ID No:
(viii-1) 112819, 180082, 365348, 366115, 667310, 713831, 726092, 730606 and 743125,
(viii-2) 430471 (SEQ ID No 404).

8. The integrated in vitro method according to claim 1, wherein, at step b), the disease-specific marker genes are selected from two or more groups of marker genes consisting of:
(i) marker genes specific for autoimmune thyroiditis (AT) that are contained as inserted DNA in the recombinant vectors selected from the group of vectors having the following IMAGE clone ID No:
(i-2) 36371, 448163, 740617, 52740, 208097, 1086914, 1929371, and 742649,
(i-1) 729542, 1837488, 284828, and 238705,
(ii) marker genes specific for Grave's disease (GD that are contained as inserted DNA in the recombinant vectors selected from the group of vectors having the following IMAGE clone ID No:
(ii-2) 136366, 726424, 178161, and 665674,
(ii-1) 23817,
(iii) marker genes specific for macrofollicular adenoma (FTA-a) that are contained as inserted DNA in the recombinant vectors selected from the group of vectors having the following IMAGE clone ID No:
(iii-2) 1420676, 739955, 745003, and 738938,
(iv) marker genes specific for atypical follicular adenoma (FTA-aty) that are contained as inserted DNA in the recombinant vectors selected from the group of vectors having the following IMAGE clone ID No:
(iv-2) 687667, and 738970,
(iv-1) 136399, and 380394,
(v) marker genes specific for microfollicular adenoma (FTA-b) that are contained as inserted DNA in the recombinant vectors selected from the group of vectors having the following IMAGE clone ID No:
(v-2) 364569, 487475, 667892, 364844, and 146976,
(v-1) 740931, and 172828,
(vi) marker genes specific for follicular carcinoma (FTC) that are contained as inserted DNA in the recombinant vectors selected from the group of vectors having the following IMAGE clone ID No:
(vi-2) 743426, 72744, 729896, and,
(vi-1) 667250, 687551, 726684, 665327, 236055, 471834, and 25077,
(vii) marker genes specific for multinodular goitres (MNG) that are contained as inserted DNA in the recombinant vectors selected from the group of vectors having the following IMAGE clone ID No:
(vii-2) 668625, 471641, 713422, 72744, and 486356,
(viii) marker genes specific for oncocytic adenoma (OTA) that are contained as inserted DNA in the recombinant vectors selected from the group of vectors having the following IMAGE clone ID No:
(viii-2) 486189, 258865, and 485104,
(viii-1) 80688,
(ix) marker genes specific for oncocytic adenoma (OTA-aty) that are contained as inserted DNA in the recombinant vectors selected from the group of vectors having the following IMAGE clone ID No:
(ix-2) 487152,
(ix-1) 26519, 1855110, and 796513,
(x) marker genes specific for oncocytic adenoma (OTC) that are contained as inserted DNA in the recombinant vectors selected from the group of vectors having the following IMAGE clone ID No:
(x-2) 40304, and 382423,
(x-1) 782688,
(xi) marker genes specific for oncocytic adenoma (PTC) that are contained as inserted DNA in the recombinant vectors selected from the group of vectors having the following IMAGE clone ID No:
(xi-2) 359051, 343987, 594279, 729542, 208097, 301122 (SEQ ID No 168), 745490, and 711918.

9. The integrated in vitro method according to claim 1, wherein step b) comprises the steps of:
b1) providing two or more sets of nucleic acids, each nucleic acid contained in a set hybridizing specifically with a nucleic acid expression product of a disease-specific marker gene comprised in one of groups (i) to (xi);
b2) reacting the sets of nucleic acids provided at step b1) with nucleic acid expression products that are previously extracted from the thyroid tissue sample provided at step a);
b3) detecting and quantifying the nucleic acid complexes formed between (i) the sets of nucleic acids provided at step b1) and (ii) the nucleic acid expression products that are extracted from the thyroid tissue sample provided at step a).

10. A kit for the in vitro prediction or diagnosis of the occurrence of a thyroid disease in a patient, which kit comprises means for quantifying the expression level of two or more disease-specific marker genes that are indicative of the risk of occurrence of, or of the occurrence of, a specific thyroid disease, wherein the said two or more disease-specific marker genes are selected from two or more of the groups of marker genes consisting of:
(i) marker genes specific for autoimmune thyroiditis (AT) that are contained as inserted DNA in the recombinant vectors selected from the group of vectors having the following IMAGE clone ID No already specified in claim 1;
(ii) marker genes specific for Grave's disease (GD that are contained as inserted DNA in the recombinant vectors selected from the group of vectors having the following IMAGE clone ID No already specified;
(iii) marker genes specific for macrofollicular adenoma (FTA-a) that are contained as inserted DNA in the recombinant vectors selected from the group of vectors having the following IMAGE clone ID No already specified;
(iv) marker genes specific for atypical follicular adenoma (FTA-aty) that are contained as inserted DNA in the recombinant vectors selected from the group of vectors having the following IMAGE clone ID No already specified;
(v) marker genes specific for microfollicular adenoma (FTA-b) that are contained as inserted DNA in the recombinant vectors selected from the group of vectors having the following IMAGE clone ID No already specified;
(vi) marker genes specific for follicular carcinoma (FTC) that are contained as inserted DNA in the recombinant vectors selected from the group of vectors having the following IMAGE clone ID No already specified;
(vii) marker genes specific for multinodular goitres (MNG) that are contained as inserted DNA in the recombinant vectors selected from the group of vectors having the following IMAGE clone ID No already specified;
(viii) marker genes specific for oncocytic adenoma (OTA) that are contained as inserted DNA in the recombinant vectors selected from the group of vectors having the following IMAGE clone ID No already specified;
(ix) marker genes specific for atypical oncocytic adenoma (OTA-aty) that are contained as inserted DNA in the recombinant vectors selected from the group of vectors having the following IMAGE clone ID No already specified;
(x) marker genes specific for oncocytic varcinoma (OTC) that are contained as inserted DNA in the recombinant vectors selected from the group of vectors having the following IMAGE clone ID No already specified; and
(xi) marker genes specific for papillary carcinoma (PTC) that are contained as inserted DNA in the recombinant vectors selected from the group of vectors having the following IMAGE clone ID No already specified.

11. A method for adapting a pharmaceutical treatment in a patient affected with a thyroid disease, wherein said method comprises the steps of:
a) performing, on at least one thyroid tissue sample collected from the said patient, the in vitro prediction or diagnosis method according to claim 1;
b) adapting the pharmaceutical treatment of the said patient by (i) increasing or decreasing the amounts of the pharmaceutical agent, (ii) combining the said pharmaceutical agent with one or more distinct pharmaceutical agent(s) or (iii) replacing the said pharmaceutical agent by one or more distinct pharmaceutical agent(s).
